(19) United States
(12) Patent Application Publication
(10) Pub. No.: US 2026/0347372 A1
(43) Pub. Date: Nov. 5, 2026

(54) SMALL MOLECULE INHIBITORS OF KRAS G12D MUTANT

(71) Applicant: MERCK SHARP & DOHME LLC, Rahway, NJ (US)

(72) Inventors: ALEC H. CHRISTIAN, Sharon, MA (US); ANTHONY DONOFRIO, Medford, MA (US); GEORGE MADALIN GIAMBASU, Natick, MA (US); SOLOMON KATTAR, Wakefield, MA (US); SHUHEI KAWAMURA, Cambridge, MA (US); BING LI, Towaco, NJ (US); MATTHEW J. MITCHELTREE, Jamaica Plain, MA (US); ANANDAN PALANI, Needham, MA (US); TOBIAS SANDMEIER, Brighton, MA (US); MICHAEL RYAN, Brookline, MA (US); DAVID L. SLOMAN, Newton, MA (US); SATORU IGUCHI, Ibaraki (JP); HITOMI KONDO, Ibaraki (JP); TOMOHIRO YAMAMOTO, Ibaraki (JP); KEIICHI SUMIYAMA, Ibaraki (JP); PATRICK SCHÖPF, London (GB)

(21) Appl. No.: 19/482,633

(22) PCT Filed: May 9, 2024
(86) PCT No.: PCT/US2024/028572

§ 371 Date: Nov. 7, 2025

Related U.S. Application Data
(60) Provisional application No. 63/500,980, filed on May 9, 2023.

(51) Int. Cl. C07D 519/00; A61K 31/519; A61K 31/551; A61K 31/553

(52) U.S. Cl. CPC C07D 519/00; A61K 31/519; A61K 31/551; A61K 31/553

(57) ABSTRACT

Compounds or their pharmaceutically acceptable salts can inhibit the G12D mutant of Kirsten rat sarcoma (KRAS) protein and are expected to have utility as therapeutic agents, for example, for treating cancer. The disclosure also provides pharmaceutical compositions which comprise compounds disclosed herein or pharmaceutically acceptable salts thereof. The disclosure also relates to methods for use of the compounds or their pharmaceutically acceptable salts in the therapy and prophylaxis of cancer and for preparing pharmaceuticals for this purpose.

### FIELD OF THE INVENTION

The present disclosure relates to certain compounds and pharmaceutically acceptable salts thereof that inhibit the G12D mutant of Kirsten rat sarcoma (KRAS) protein and are expected to have utility as therapeutic agents, for example, for treatment of cancer. The present application also relates to pharmaceutical compositions containing such compounds as well as methods of using the compounds for treating cancer.

### BACKGROUND

Mutations in any one of the three main isoforms of RAS (HRAS, NRAS, or KRAS) genes are among the most common events in human tumorigenesis. KRAS mutations are detected in 25-30% of tumors. By comparison, the rates of oncogenic mutation occurring in the NRAS and HRAS family members are much lower (8% and 3% respectively).

Ras proteins are membrane-associated guanine nucleotide-binding proteins which function as molecular switches. Ras proteins function as components of signaling pathways transmitting signals from cell-surface receptors to regulate cellular proliferation, survival and differentiation. Ras proteins cycle between an inactive GDP-bound state and an active GTP-bound state.

Exchange of a glycine for an aspartate at residue 12 of RAS (the G12D mutation) results from a gain-of-function mutation commonly found in RAS gene. The K-Ras(G12D) mutation represents the highest frequency of KRAS mutations. Wan Y. et al., Front. Oncol. 2020; 10:1326. The K-Ras (G12D) mutation appears frequently in certain cancer types including pancreatic ductal adenocarcinoma, colorectal cancer and lung adenocarcinoma. Pancreatic ductal adenocarcinoma is particularly significant. This cancer type represents a malignancy having a 5-year survival rate of less than 8% and is also the fourth leading cause of cancer-related deaths in the western world. See Schneeweis C. et al., Small GTPases 2018; 9(6) 457-464.

Accordingly, while progress has been made in this field, there remains a need in the art for improved compounds and methods for treatment of cancer, for example, by inhibition of a mutant KRAS protein (e.g., KRAS G12D). Embodiments of the present disclosure fulfill this need and provide further related advantages.

### SUMMARY OF THE DISCLOSURE

The present disclosure provides small molecule inhibitors which modulate mutant KRAS proteins and may be valuable pharmaceutically active compounds for the treatment of cancer. In some embodiments the disclosed compounds selectively inhibit the KRAS (G12D) protein. The compounds of the disclosure, including compounds of Formula (I):

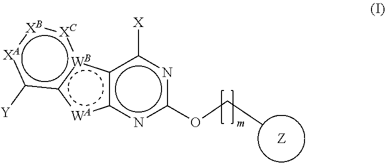

and their pharmaceutically acceptable salts, can modulate the activity of KRAS activity and thereby affect the signaling pathway which regulates cell growth, differentiation, and proliferation associated with oncological disorders. In certain embodiments, the compounds of the disclosure can inhibit the KRAS (G12D) protein. The disclosure furthermore provides processes for preparing compounds of the disclosure, methods for using such compounds to treat oncological disorders, and pharmaceutical compositions which comprise compounds of the disclosure.

### DETAILED DESCRIPTION OF THE INVENTION

### Compounds of the Disclosure

In one embodiment, the present disclosure provides a compound having structural Formula (I), or a pharmaceutically acceptable salt thereof, as shown above, wherein:
X is:

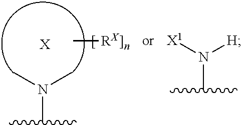

Ring X is selected from the group consisting of
   (i) a 6- to 9-membered bridged bicyclic heterocyclalkyl, wherein the bridged bicyclic heterocycloalkyl is saturated and contains one N atom and one optional O atom in addition to the illustrated N atom;
   (ii) an 8- to 10-membered fused heterocycloalkyl, wherein the fused heterocycloalkyl is saturated and contains one N atom in addition to the illustrated N atom;
   each R^{X} is independently selected from the group consisting of fluoro, cyano, hydroxy, oxo, C₁-C₆ alkyl, C₁-C₆ fluoroalkyl, C₁-C₆ cyanoalkyl and C₁-C₃ hydroxyalkyl;
X¹ is —(CH₂)ₚ—C^{X}, wherein C^{X} is:
   (i) a 3- to 10-membered mono- or bicyclic cycloalkyl;
   (ii) a 3- to 10-membered mono- or bicyclic heterocycloalkyl, wherein the 3- to 10-membered mono- or bicyclic heterocycloalkyl is saturated and contains from 1 to 3 heteroatoms independently selected from the group consisting of N, S, and O;
   wherein C^{X} is substituted by one R^{C1} which is selected from the group consisting of amino, C₁-C₃ alkylamino, and C₁-C₃ dialkylamino;
      wherein C^{X} is unsubstituted or substituted by 1-3 R^{C2} substituents selected from the group consisting of fluoro, cyano, hydroxy, oxo, C₁-C₆ alkyl, C₁-C₆ fluoroalkyl, and C₁-C₃ hydroxyalkyl;
X^{A}, X^{B}, and X^{C} are independently selected from the group consisting of N, C(H), and C(R¹);
each R¹ is independently selected from the group consisting of C₁-C₃ alkyl, C₁-C₃ fluoroalkyl, halo and cyano;
W^{A} is selected from the group consisting of N(R^{W1}), C(R^{W2})₂, O, S, Se, and C(R^{W3});
R^{W1} is selected from the group consisting of C₁-C₃ alkyl, C₁-C₃ fluoroalkyl, C₃-C₆ cycloalkyl, and C₃-C₆ fluorocycloalkyl;
each R^{W2} is independently selected from the group consisting of H, halo, hydroxy, C₁-C₃ alkyl, and C₁-C₃ fluoroalkyl;
   or alternatively, the two R^{W2}, together with the carbon atom to which they are attached form a C₃-C₆ cycloalkyl or C₃-C₆ fluorocycloalkyl;
R^{W3} is selected from the group consisting of H, halo, C₁-C₃ alkyl, C₁-C₃ alkoxy, C₁-C₃ fluoroalkyl, C₁-C₃ fluoroalkoxy, C₃-C₆ cycloalkyl, and C₃-C₆ fluorocycloalkyl;
W^{B} is C or N, wherein when:
   W^{B} is C, then W^{A} is N(R^{W1}), C(R^{W2})₂, O, S, or Se; and
   W^{B} is N, then W^{A} is C(R^{W3});
Y is C₁-C₆ alkyl, C₁-C₆ fluoroalkyl, or C^{y};
C^{y} is
   (i) an aryl selected from the group consisting of phenyl or naphthyl:
   (ii) a 5- to 6-membered monocyclic heteroaryl, wherein the 5- to 6-membered monocyclic heteroaryl contains 1 to 3 heteroatoms independently selected from the group consisting of N, S, and O;
   (iii) a 9- to 10-membered bicyclic heteroaryl, wherein the 9- to 10-membered bicyclic heteroaryl contains 1 to 3 heteroatoms independently selected from the group consisting of N, O, and S; or
   (iv) a 12- to 17-membered tri- or tetracyclic heterocycloalkyl, where at least 2 of the rings of the 12- to 17-membered tri- or tetracyclic heterocycloalkyl are aromatic, the third ring is partially unsaturated or aromatic, and the fourth ring, if present, is saturated, wherein the 12- to 17-membered tri- or tetracyclic heterocycloalkyl contains 1 to 4 heteroatoms independently selected from the group consisting of N, S, and O;
      wherein C^{y} is unsubstituted or substituted by 1 to 4 R^{Y} substituents independently selected from the group consisting of halo, hydroxy, C₁-C₃ alkyl, C₂-C₄ alkynyl, C₁-C₃ fluoroalkyl, C₁-C₃ alkoxy, C₁-C₃ haloalkoxy, C₁-C₃ alkylthio, C₁-C₃ fluoroalkylthio, amino, C₁-C₃ alkylamino, C₁-C₃ dialkylamino, C₃-C₁₂ cycloalkyl, C₃-C₁₂ fluorocycloalkyl, tri(C₁-C₃ alkyl)silyl, and cyano;
Ring Z is:
   (i) a 3- to 10-membered mono- or bicyclic cycloalkyl;
   (ii) a 3- to 10-membered mono- or bicyclic-heterocycloalkyl, wherein the 3- to 10-membered mono- or bicyclic-heterocycloalkyl is saturated and contains 1 to 2 heteroatom groups selected from the group consisting of N, S, S(O), S(O)₂ and O; or
   (iii) a 3- to 10-membered spiroheterocycloalkyl, wherein the 3- to 10-membered spiroheterocycloalkyl is saturated and contains 1 to 2 heteroatoms selected from the group consisting of N, S, and O;
      wherein Ring Z is unsubstituted or substituted by 1 to 4 R^{Z} substituents independently selected from the group consisting of halo, hydroxy, oxo, C₁-C₆ alkyl, C₁-C₃ alkoxy, C₁-C₃ hydroxyalkyl, C₁-C₆ fluoroalkyl, C₁-C₃ alkylsulfonyl, C₁-C₃ alkylsulfinyl, C₁-C₃ alkylsulfonimidoyl, carboxy, carbamoyl, cyano, methoxy(C₁-C₃)alkyl, amino(C₁-C₃)alkyl, C₁-C₃ alkylamino(C₁-C₃)alkyl, C₁-C₃ dialkylamino, C₁-C₃ dialkylamino(C₁-C₃)alkyl, azetidinyl(C₁-C₃)alkyl, pyrollidinyl(C₁-C₃)alkyl, piperidinyl(C₁-C₃)alkyl, heterocyclyl(C₁-C₃)alkyl, and fluoromethylenyl;
         or alternatively, two R^{Z}, together with the carbon atom to which they are attached form a C₃-C₆ cycloalkyl or C₃-C₆ fluorocycloalkyl;
         wherein the heterocyclyl group of heterocyclyl(C₁-C₃)alkyl is:
         (i) a 3- to 10-membered mono- or bicyclic-heterocyclyl group, wherein the 3- to 10-membered mono- or bicyclic-heterocyclyl group is saturated and contains 1 to 2 heteroatom groups selected from the group consisting of N, S, S(O), S(O)₂ and O; or
         (ii) a 3- to 12-membered spiroheterocyclyl group, wherein the 3- to 10-membered spiroheterocyclyl group is saturated and contains 1 to 2 heteroatoms selected from the group consisting of N, S, and O;
         wherein the heterocyclyl group of heterocyclyl(C₁-C₃)alkyl is unsubstituted or substituted by 1 to 4 R^{HC} substituents independently selected from the group consisting of halo, acyl, C₁-C₆ fluoroalkyl, C₁-C₆ alkoxyalkyl, and methyltriazolyl(C₁-C₃)alkyl;
subscript m is 0, 1, or 2;
subscript n is 0, 1, 2, or 3; and
subscript p is 0 or 1.

In another embodiment, the present disclosure provides a compound having structural Formula (I), or a pharmaceutically acceptable salt thereof, as shown above, wherein:
X is:

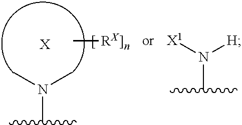

Ring X is selected from the group consisting of
   (i) a 6- to 9-membered bridged bicyclic heterocycloalkyl, wherein the bridged bicyclic heterocycloalkyl is saturated and contains one N atom and one optional O atom in addition to the illustrated N atom;
   (ii) an 8- to 10-membered fused heterocycloalkyl, wherein the fused heterocycloalkyl is saturated and contains one N atom in addition to the illustrated N atom;
   each R^{X} is independently selected from the group consisting of fluoro, cyano, hydroxy, oxo, C₁-C₆ alkyl, C₁-C₆ fluoroalkyl, and C₁-C₃ hydroxyalkyl;
X¹ is —(CH₂)ₚ—C^{X}, wherein C^{X} is:
   (i) a 3- to 10-membered mono- or bicyclic cycloalkyl;
   (ii) a 3- to 10-membered mono- or bicyclic heterocycloalkyl, wherein the 3- to 10-membered mono- or bicyclic heterocycloalkyl is saturated and contains from 1 to 3 heteroatoms independently selected from the group consisting of N, S, and O;
   wherein C^{X} is substituted by one R^{C1} which is selected from the group consisting of amino, C₁-C₃ alkylamino, and C₁-C₃ dialkylamino;
      wherein C^{X} is unsubstituted or substituted by 1-3 R^{C2} substituents selected from the group consisting of fluoro, cyano, hydroxy, oxo, C₁-C₆ alkyl, C₁-C₆ fluoroalkyl, and C₁-C₃ hydroxyalkyl;
X^{A}, X^{B}, and X^{C} are independently selected from the group consisting of N, C(H), and C(R¹);
each R¹ is independently selected from the group consisting of C₁-C₃ alkyl, C₁-C₃ fluoroalkyl, halo and cyano;
W^{A} is selected from the group consisting of N(R^{W1}), C(R^{W2})₂, O, S, Se, and C(R^{W3});
R^{W1} is selected from the group consisting of C₁-C₃ alkyl, C₁-C₃ fluoroalkyl, C₃-C₆ cycloalkyl, and C₃-C₆ fluorocycloalkyl;
each R^{W2} is independently selected from the group consisting of H, halo, hydroxy, C₁-C₃ alkyl, and C₁-C₃ fluoroalkyl;
   or alternatively, the two R^{W2}, together with the carbon atom to which they are attached form a C₃-C₆ cycloalkyl or C₃-C₆ fluorocycloalkyl;
R^{W3} is selected from the group consisting of H, halo, C₁-C₃ alkyl, C₁-C₃ alkoxy, C₁-C₃ fluoroalkyl, C₁-C₃ fluoroalkoxy, C₃-C₆ cycloalkyl, and C₃-C₆ fluorocycloalkyl;
each R^{W4} is independently selected from the group consisting of H, halo, and C₁-C₃ alkyl, or alternatively two R^{W4}, together with the carbon atom to which are attached form a 3- to 6-membered cycloalkyl;
W^{B} is C or N, wherein when:
   W^{B} is C, then W^{A} is N(R^{W1}), C(R^{W2})₂, O, or S; and
   W^{B} is N, then W^{A} is C(R^{W3});
Y is C₁-C₆ alkyl, C₁-C₆ fluoroalkyl, or C^{y};
C^{y} is
   (i) an aryl selected from the group consisting of phenyl or naphthyl:
   (ii) a 5- to 6-membered monocyclic heteroaryl, wherein the 5- to 6-membered monocyclic heteroaryl contains 1 to 3 heteroatoms independently selected from the group consisting of N, S, and O;
   (iii) a 9- to 10-membered bicyclic heteroaryl, wherein the 9- to 10-membered bicyclic heteroaryl contains 1 to 3 heteroatoms independently selected from the group consisting of N, O, and S; or
   (iv) a 12- to 17-membered tri- or tetracyclic heterocycloalkyl, where at least 2 of the rings of the 12- to 17-membered tri- or tetracyclic heterocycloalkyl are aromatic, the third ring is partially unsaturated or aromatic, and the fourth ring, if present, is saturated, wherein the 12- to 17-membered tri- or tetracyclic heterocycloalkyl contains 1 to 4 heteroatoms independently selected from the group consisting of N, S, and O;
      wherein C^{y} is unsubstituted or substituted by 1 to 4 R^{Y} substituents independently selected from the group consisting of halo, hydroxy, C₁-C₃ alkyl, C₂-C₄ alkynyl, C₁-C₃ fluoroalkyl, C₁-C₃ alkoxy, C₁-C₃ haloalkoxy, C₁-C₃ alkylthio, C₁-C₃ fluoroalkylthio, amino, C₁-C₃ alkylamino, C₁-C₃ dialkylamino, C₃-C₁₂ cycloalkyl, C₃-C₁₂ fluorocycloalkyl, tri(C₁-C₃ alkyl)silyl, and cyano;
Ring Z is:
   (i) a 3- to 10-membered mono- or bicyclic cycloalkyl;
   (ii) a 3- to 10-membered mono- or bicyclic-heterocycloalkyl, wherein the 3- to 10-membered mono- or bicyclic-heterocycloalkyl is saturated and contains 1 to 2 heteroatom groups selected from the group consisting of N, S, S(O), S(O)₂ and O; or
   (iii) a 3- to 10-membered spiroheterocycloalkyl, wherein the 3- to 10-membered spiroheterocycloalkyl is saturated and contains 1 to 2 heteroatoms selected from the group consisting of N, S, and O;
      wherein Ring Z is unsubstituted or substituted by 1 to 4 R^{Z} substituents independently selected from the group consisting of halo, hydroxy, oxo, C₁-C₆ alkyl, C₁-C₃ alkoxy, C₁-C₃ hydroxyalkyl, C₁-C₆ fluoroalkyl, C₁-C₃ alkylsulfonyl, C₁-C₃ alkylsulfinyl, C₁-C₃ alkylsulfonimidoyl, carboxy, carbamoyl, cyano, methoxy(C₁-C₃)alkyl, amino(C₁-C₃)alkyl, C₁-C₃ alkylamino(C₁-C₃)alkyl, C₁-C₃ dialkylamino, C₁-C₃ dialkylamino(C₁-C₃)alkyl, azetidinyl(C₁-C₃)alkyl, pyrollidinyl(C₁-C₃)alkyl, piperidinyl(C₁-C₃)alkyl, and fluoromethylenyl;
         or alternatively, two R^{Z}, together with the carbon atom to which they are attached form a C₃-C₆ cycloalkyl or C₃-C₆ fluorocycloalkyl;
subscript m is 0, 1, or 2;
subscript n is 0, 1, 2, or 3; and
subscript p is 0 or 1.

In another embodiment, the present disclosure provides a compound of Formula (I), or a pharmaceutically acceptable salt thereof, wherein X is

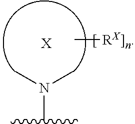

In another embodiment, the present disclosure provides a compound of Formula (I), or a pharmaceutically acceptable salt thereof, wherein

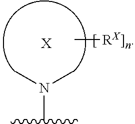

is selected from the group consisting of

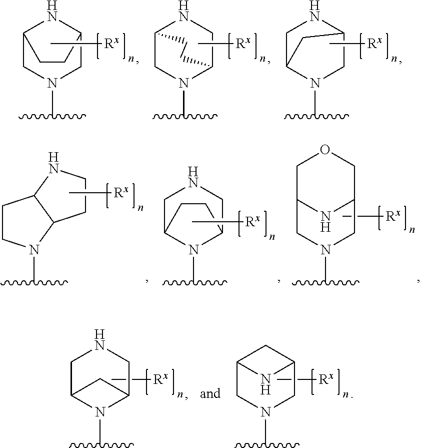

In another embodiment, the present disclosure provides a compound of Formula (I), or a pharmaceutically acceptable salt thereof, wherein X is

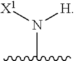

In another embodiment, the present disclosure provides a compound of Formula (I), or a pharmaceutically acceptable salt thereof, wherein X¹ is —(CH₂)ₚ—C^{X}, wherein:
C^{X} is

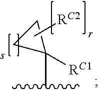

subscript p is 1;
subscript r is 0, 1, 2, or 3; and
subscript s is 1, 2, 3, 4, or 5,
   wherein:
   R^{C1} is C₁-C₃ dialkylamino; and
each R^{C2} is independently selected from the group consisting of fluoro, cyano, hydroxy, oxo, C₁-C₆ alkyl, C₁-C₆ fluoroalkyl, and C₁-C₃ hydroxyalkyl.

In another embodiment, the present disclosure provides a compound of Formula (I), or a pharmaceutically acceptable salt thereof, wherein W^{A} is N(R^{W1}).

In another embodiment, the present disclosure provides a compound of Formula (I), or a pharmaceutically acceptable salt thereof, wherein R^{W1} is methyl.

In another embodiment, the present disclosure provides a compound of Formula (I), or a pharmaceutically acceptable salt thereof, wherein W^{A} is O.

In another embodiment, the present disclosure provides a compound of Formula (I), or a pharmaceutically acceptable salt thereof, wherein W^{A} is C(R^{W2})₂.

In another embodiment, the present disclosure provides a compound of Formula (I), or a pharmaceutically acceptable salt thereof, wherein W^{A} is S.

In another embodiment, the present disclosure provides a compound of Formula (I), or a pharmaceutically acceptable salt thereof, wherein:
X^{A} is N,
X^{B} and X^{C} are independently selected from the group consisting of C(H) and (CR¹); and
W^{B} is C.

In another embodiment, the present disclosure provides a compound of Formula (I), or a pharmaceutically acceptable salt thereof, wherein the compound of Formula (I) has the Formula (IA)

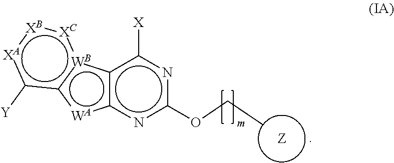

In another embodiment, the present disclosure provides a compound of Formula (I), or a pharmaceutically acceptable salt thereof, wherein the compound of Formula (I) has the Formula (IB)

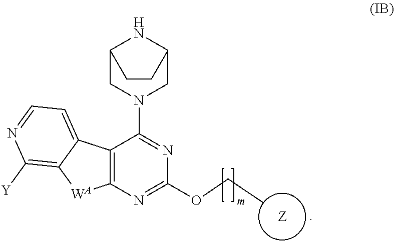

In another embodiment, the present disclosure provides a compound of Formula (I), or a pharmaceutically acceptable salt thereof, wherein W^{A} is S or N(CH₃).

In another embodiment, the present disclosure provides a compound of Formula (I), or a pharmaceutically acceptable salt thereof, wherein the group is

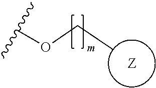

selected from the group consisting of:

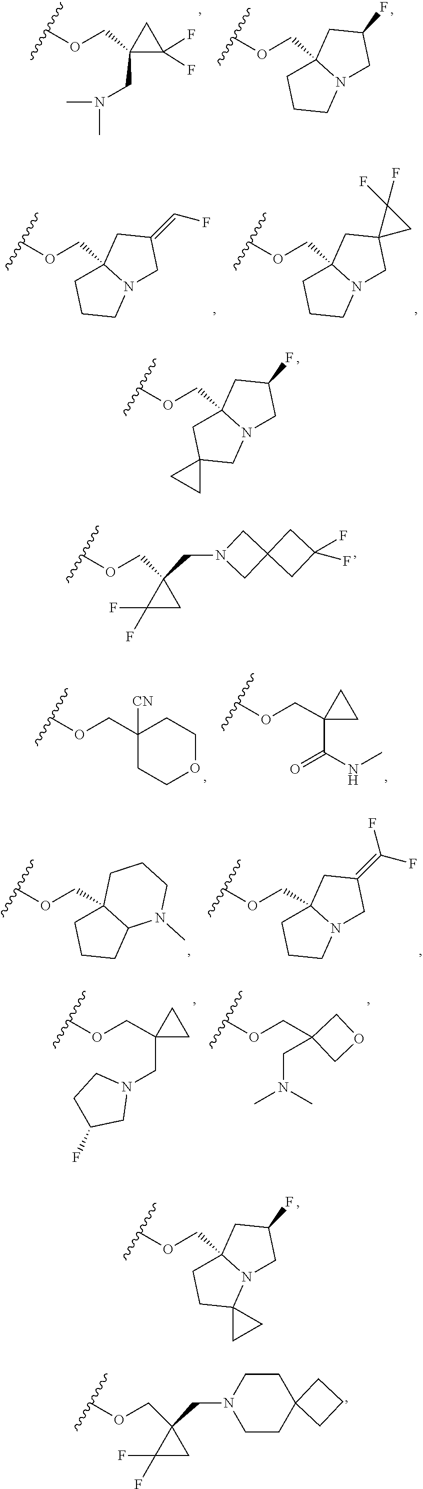

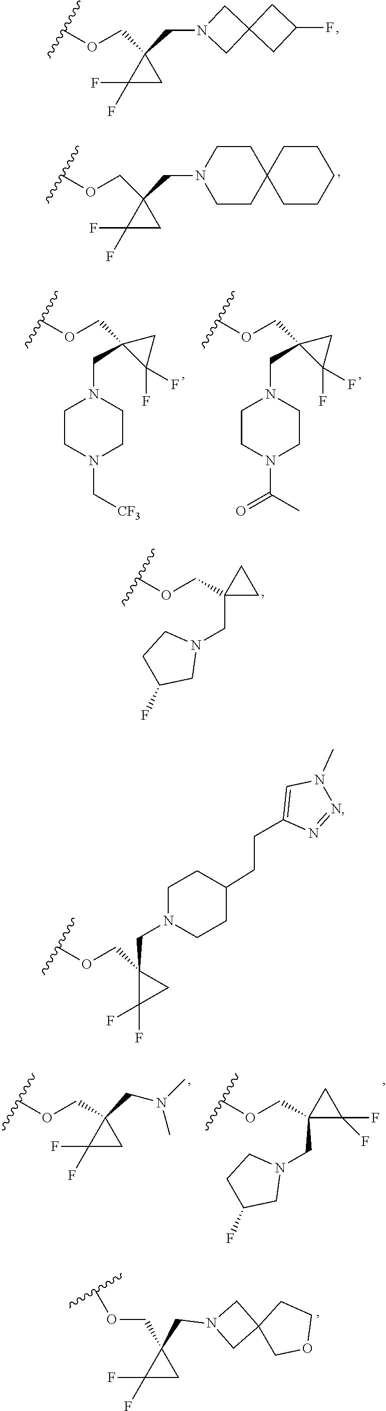

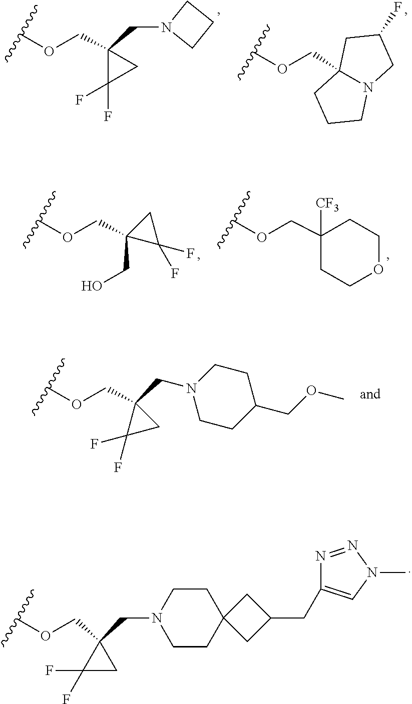 [SMILES: *OC[C@]1(CN2CCC2)CC1(F)F ; *OC[C@@]12CCCN1C[C@@H](F)C2 ; *OC[C@]1(CO)CC1(F)F ; *OCC1(C(F)(F)F)CCOCC1 ; *OC[C@]1(CN2CCC(COC)CC2)CC1(F)F ; *OC[C@]1(CN2CCC3(CC2)CC(Cc2cn(C)nn2)C3)CC1(F)F]

In another embodiment, the present disclosure provides a compound of Formula (I), or a pharmaceutically acceptable salt thereof, wherein the group

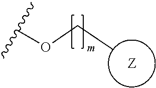

is selected from the group consisting of:

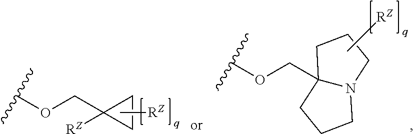

wherein subscript q is 1, 2, 3 or 4.

In another embodiment, the present disclosure provides a compound of Formula (I), or a pharmaceutically acceptable salt thereof, wherein the group

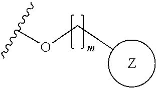

is selected from the group consisting of:

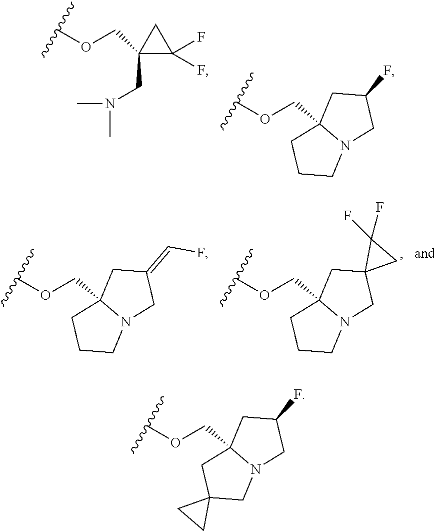

In another embodiment, the present disclosure provides a compound of Formula (I), or a pharmaceutically acceptable salt thereof, wherein the group

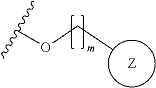

is selected from the group consisting of:

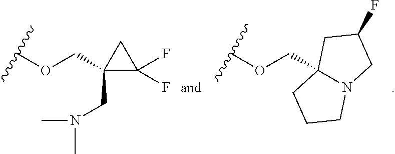 [SMILES: *OC[C@]1(CN(C)C)CC1(F)F ; *OC[C@@]12CCCN1C[C@H](F)C2]

In another embodiment, the present disclosure provides a compound of Formula (I), or a pharmaceutically acceptable salt thereof, wherein Y is C^{y}, and C^{y} is unsubstituted or substituted phenyl, naphthyl, pyridyl, indazolyl, benzothienyl, benzoxazolyl, benzothiazolyl, or isoquinolinyl.

In another embodiment, the present disclosure provides a compound of Formula (I), or a pharmaceutically acceptable salt thereof, wherein C^{y} is naphthyl, indazolyl, pyridyl, benzothienyl, wherein C^{y} is substituted by 1 to 3 R^{Y} substituents independently selected from the group consisting of halo, hydroxy, amino, C₁-C₃ alkyl, C₁-C₃ fluoroalkyl, C₃-C₆ cycloalkyl, C₃-C₆ fluorocycloalkyl, C₂-C₄ alkynyl, and cyano.

In another embodiment, the present disclosure provides a compound of Formula (I), or a pharmaceutically acceptable salt thereof, wherein C^{Y} is selected from the group consisting of

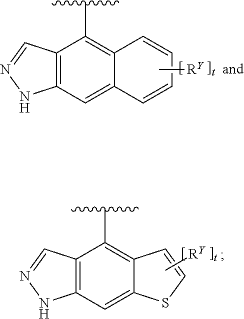

and subscript t is 0, 1, 2, 3, or 4.

In specific embodiments, the present disclosure provides a compound as described in any one of Examples 1-78 as set forth below, or a pharmaceutically acceptable salt thereof.

In specific embodiments, the present disclosure provides a compound as described in any one of Examples 1-80 and 82-118 as set forth below, or a pharmaceutically acceptable salt thereof.

In a specific embodiment, the present disclosure provides a compound of formula

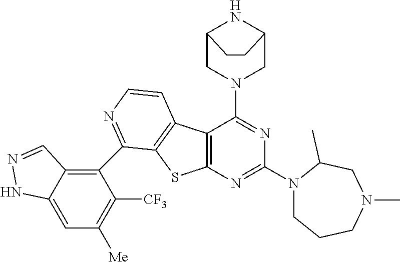 [SMILES: Cc1cc2[nH]ncc2c(-c2nccc3c2sc2nc(N4CCCN(C)CC4C)nc(N4CC5CCC(C4)N5)c23)c1C(F)(F)F]

or a pharmaceutically acceptable salt thereof.

The present disclosure includes the pharmaceutically acceptable salts of the compounds defined herein, including the pharmaceutically acceptable salts of all structural formulas, embodiments and classes defined herein.

### Definitions

Unless defined otherwise, all technical and scientific terms used herein have the same meaning as is commonly understood by one of skill in the art to which this invention belongs.

As used throughout this disclosure, “compound(s) of Formula (I)”, “compound(s) disclosed herein”, “compound(s) described herein”, “compound(s) of the disclosure”, etc., are used interchangeably and are to be understood to include the disclosed compounds of Formula (I). The compounds of Formula (I) can form salts which are also within the scope of the present disclosure. Reference to a compound of the disclosure (or compound of Formula (I)) herein is understood to include reference to salts thereof, unless otherwise indicated.

“Acyl” refers to a moiety derived by the removal of one or more hydroxyl groups from an oxoacid. An acyl group contains a central carbon atom, a double-bonded oxygen atom to the central carbon atom, and a single-bonded alkyl group to the central carbon atom.

“Alkenyl” means an aliphatic hydrocarbon group containing at least one carbon-carbon double bond and which may be straight or branched. Non-limiting examples include ethenyl, propenyl, and butenyl.

“Alkenylenyl” means an aliphatic hydrocarbon group containing at least one carbon-carbon double bond radical. For example, methylenyl (CH₂═) is included.

“Alkyl”, as well as other groups having the prefix “alk”, such as alkoxy, and the like, means carbon chains which may be linear or branched, or combinations thereof, containing the indicated number of carbon atoms. For instance, a C₁-C₆ alkyl means an alkyl group having one (i.e., methyl) up to 6 carbon atoms (i.e., hexyl). In particular embodiments, linear alkyl groups have 1-6 carbon atoms and branched alkyl groups have 3-7 carbon atoms. Examples of alkyl groups include methyl, ethyl, propyl, isopropyl, butyl, sec- and tert-butyl, pentyl, hexyl, heptyl, octyl, nonyl and the like.

“Alkylamino” means an alkyl group linked to an amine, wherein the nitrogen atom is substituted by one or more alkyl substituents. The bond to the parent group is through the nitrogen atom of the amino component.

“Alkylsulfinyl” means an alkyl group linked to a sulfinyl, wherein the sulfur atom is substituted by an alkyl substituent. The bond to the parent group is through the sulfur atom of the group.

“Alkylsulfonimidoyl” means an alkyl group linked to a sulfonimidoyl (i.e., sulfur atom having a double bond with an oxygen atom and a double bond with a nitrogen atom), wherein the sulfur atom is substituted by an alkyl substituent, i.e., alkyl-S(O)(N)—. The bond to the parent group is through the sulfur atom of the group.

“Alkylsulfonyl” means an alkyl group linked to a sulfonyl, wherein the sulfur atom is substituted by an alkyl substituent. The bond to the parent group is through the sulfur atom of the group.

“Alkylthio” means an alkyl group linked to a sulfur. “Fluoroalkylthio” means an alkylthio that is mono-or multiple-fluoro-substituted. The bond to the parent group is through the sulfur atom of the group.

“Alkoxy” and “alkyl-O—” are used interchangeably and refer to an alkyl group linked to oxygen. “Haloalkoxy”, e.g., fluoroalkoxy, means an alkoxy that is mono-or multiple-halo-substituted. The bond to the parent group is through the oxygen atom of the group.

“Alkoxyalkyl”, e.g., methoxyalkyl, means an alkoxy group linked to an alkyl. The bond to the parent group is through the carbon atom of the alkyl component.

“Alkynyl” means an aliphatic hydrocarbon group containing at least one carbon-carbon triple bond and which may be straight or branched. Non-limiting examples include ethynyl, propynyl, and butynyl.

“Amino” means an amine group that contains two substituents bonded to a nitrogen atom via two single covalent bonds. The bond to the parent group is through the nitrogen atom of the group.

“Aminoalkyl” means an amino group linked to an alkyl, wherein one of the carbon atoms of the alkyl is substituted an amino substituent. The bond to the parent group is through one of the carbon atoms of the alkyl component.

“Aryl” means a monocyclic, bicyclic, tricyclic, or tetracyclic carbocyclic aromatic ring or ring system containing 5-17 carbon atoms, wherein at least one of the rings is aromatic. Non-limiting examples include phenyl and naphthyl.

“Azetidinylalkyl” means an azetidinyl group linked to an alkyl, wherein one of the carbon atoms of the alkyl is substituted by an azetidinyl substituent. The bond to the parent group is through one of the carbon atoms of the alkyl component.

“Bicyclic ring system” refers to two joined rings. “Tricyclic ring system” refers to three joined rings. “Tetracyclic ring system” refers to four joined rings. The rings may be fused, i.e., share two adjacent atoms, or “spirocyclic”, i.e., share only a single atom, or “bridged”, i.e., share three or more atoms with two bridgehead atoms being connected by a bridge containing at least one atom. Likewise the bicyclic or tricyclic rings may be aryl rings, heterocyclic rings, cycloalkyl rings, etc.

“Carbamoyl” means a H₂N—C(O)— group, which is the univalent group formed by loss of —OH group of carbamic acid. The bond to the parent group is through the carbon atom of the carbonyl component.

“Carboxy” means an HO₂C— group. The bond to the parent group is through the carbon atom of the carbonyl component.

“Cycloalkyl” means a saturated cyclic hydrocarbon radical. In particular embodiments, the cycloalkyl group has 3-12 carbon atoms, forming 1-3 carbocyclic rings, wherein cyclic systems having 2-3 rings can be fused. Examples of cycloalkyl include cyclopropyl, cyclobutyl, cyclopentyl, cyclohexyl, cycloheptyl, adamantyl, and the like. “Fluorocycloalkyl” means a saturated cyclic hydrocarbon radical that is mono- or multiple-fluoro-substituted, e.g., doubly fluoro-substituted cyclopentyl. “Cycloalkoxy” refers to a cycloalkyl group linked through an oxygen to the parent moiety. “Spirocycloalkyl” means a saturated spirocyclic hydrocarbon radical having at least two rings sharing only a single atom.

“Cyano” means a N≡C— group. The bond to the parent group is through the carbon atom.

“Cyanoalkyl” means a cyano group linked to an alkyl. The bond to the parent group is through a carbon atom of the alkyl component.

“Dialkylamino” means an alkylamino as previously defined, wherein the nitrogen atom of the amine is substituted by two alkyl substituents, which substitutions can be the same or different, e.g., —N(CH₃)₂ or —N(CH₃)(CH₂CH₃). The bond to the parent group is through the nitrogen atom of the amino component.

“Fluoroalkyl” includes mono-substituted as well as multiple fluoro-substituted alkyl groups, up to perfluoro substituted alkyl. For example, fluoromethyl, 1,1-difluoroethyl, trifluoromethyl or 1,1,1,2,2-pentafluorobutyl are included. The bond to the parent group is through one of the carbon atoms of the alkyl component.

“Fluoroalkenylenyl” includes mono-substituted as well as multiple fluoro-substituted alkenylenyl groups. For example, fluoromethylenyl (F—CH═) is included.

“Halogen” or “halo”, unless otherwise indicated, includes fluorine (fluoro), chlorine (chloro), bromine (bromo) and iodine (iodo). In one embodiment, halo is fluoro (—F) or chloro (—Cl).

“Heteroaryl” refers to aromatic monocyclic, bicyclic and tricyclic ring structures in which one or more atoms in the ring, the heteroatom(s), is an element other than carbon. Heteroatoms are typically O, S, or N atoms. Examples of heteroaryl groups include pyrazolyl, oxadiazolonyl, pyridinyl, pyrimidinyl, pyrrolyl, pyridazinyl, isoxazolyl, thiazolyl, oxazolyl, indolyl, benzoxazolyl, benzothiazolyl, and imidazolyl.

“Heterocycloalkyl” or “heterocyclyl” or “heterocyclic ring” or “heterocyclyl ring” means a partially aromatic or non-aromatic monocyclic, bicyclic, tricyclic or tetracyclic ring system comprising about 3 to about 17 ring atoms, preferably about 5 to about 10 ring atoms, in which one or more of the atoms in the ring system is an element other than carbon, for example, nitrogen, oxygen, phosphorus or sulfur, alone or in combination. There are no adjacent oxygen and/or sulfur atoms present in the ring system. In some embodiments, heterocycloalkyls contain about 5 to about 6 ring atoms. The prefix aza, oxa, phospha or thia before the heterocyclyl root name means that at least a nitrogen, oxygen, phosphorus or sulfur atom respectively is present as a ring atom. In some embodiments, the nitrogen or sulfur atom of the heterocycloalkyl can be optionally oxidized to the corresponding N-oxide, S-oxide or S,S-dioxide. For instance, in some embodiments the heterocycloalkyl can contain N, S, S(O), S(O)₂ and/or O (which are referred to herein as “heteroatom groups”). Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, phosphorinane, phosphinane, 1-oxophosphinan-1-ium and the like. “Spiroheterocycloalkyl” or “spiroheterocyclyl” refers to a fused ring system in which the rings share only a single atom and at least one of the rings is a heterocyclyl.

“Hydroxy” means a HO— group in which the bond to the parent moiety is through the oxygen atom.

“Hydroxyalkyl” means a HO-alkyl- group in which alkyl is as previously defined. The bond to the parent moiety is through one of the carbon atoms of the alkyl component. Preferred hydroxyalkyls contain lower alkyl. Non-limiting examples of suitable hydroxyalkyl groups include hydroxymethyl and 2-hydroxyethyl. “Hydroxyfluoroalkyl” means a HO-fluoroalkyl- group in which fluoroalkyl is as previously defined. “Hydroxycycloalkyl” means a HO-cycloalkyl- group in which cycloalkyl is as previously defined. “Hydroxyfluorocycloalkyl” means a HO-fluorocycloalkyl- group in which fluorocycloalkyl is as previously defined.

“Methyltriazolylalkyl” means a methyl group linked to a triazolyl group linked to an alkyl. The bond to the parent group is through a carbon atom of the alkyl component.

“Oxo” means an oxygen atom double bonded to the parent moiety.

“Piperidinylalkyl” means a piperidinyl group linked to an alkyl, wherein one or more carbon atoms of the alkyl is substituted by one or more piperidinyl substituents. The bond to the parent group is through one of the carbon atoms of the alkyl component.

“Pyrollidinylalkyl” means a pyrollidinyl group linked to an alkyl, wherein one or more carbon atoms of the alkyl is substituted by one or more pyrollidinyl substituents. The bond to the parent group is through one of the carbon atoms of the alkyl component.

“Trialkylsilyl” means a silicon radical having three alkyl groups covalently bonded to the silicon atom. The alkyl groups can be the same or different. The bond to the parent group is through the silicon atom of the group.

When any variable (e.g., R^{X}) occurs more than one time in any constituent or in Formula (I) or other generic formulas herein, its definition on each occurrence is independent of its definition at every other occurrence. Combinations of substituents and/or variables are permissible only if such combinations result in stable compounds. In choosing compounds of the present disclosure, one of ordinary skill in the art will recognize that the various substituents, e.g., R^{X}, are to be chosen in conformity with well-known principles of chemical structure connectivity and stability. Unless expressly stated to the contrary, substitution by a named substituent is permitted on any atom in a ring (e.g., aryl, a heteroaryl ring, or a saturated heteroaryl ring) provided such ring substitution is chemically allowed and results in a stable compound. A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject).

The term “substituted” shall be deemed to include multiple degrees of substitution by a named substituent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

Unless expressly depicted or described otherwise, variables depicted in a structural formula with a “floating” bond, such as R^{X}, are permitted on any available carbon atom in the ring to which the variable is attached. When a moiety is noted as being “optionally substituted” in Formula (I) or any embodiment thereof, it means that Formula (I) or the embodiment thereof encompasses compounds that contain the noted substituent (or substituents) on the moiety and also compounds that do not contain the noted substituent (or substituents) on the moiety.

The wavy line [symbol] , as used herein, indicates a point of attachment to the rest of the compound.

The compounds of Formula (I) may contain one or more asymmetric centers and can thus occur as racemates and racemic mixtures, single enantiomers, diastereoisomeric mixtures and individual diastereoisomers. Centers of asymmetry that are present in the compounds of Formula (I) can all independently of one another have S configuration or R configuration. The compounds of Formula (I) include all possible enantiomers and diastereomers and mixtures of two or more stereoisomers, for example, mixtures of enantiomers and/or diastereomers, in all ratios. Thus, enantiomers are a subject of the disclosure in enantiomerically pure form, both as levorotatory and as dextrorotatory antipodes, in the form of racemates and in the form of mixtures of the two enantiomers in all ratios. In the case of a cis/trans isomerism, the disclosure includes both the cis form and the trans form as well as mixtures of these forms in all ratios. The present disclosure is meant to comprehend all such stereoisomeric forms of the compounds of Formula (I). Where a structural formula or chemical name specifies a particular configuration at a stereocenter, the enantiomer or stereoisomer of the compound resulting from that specified stereocenter is intended. Where a structural formula of the compounds of Formula (I) indicates a straight line at a chiral center, the structural formula includes both the S and R stereoisomers associated with the chiral center and mixtures thereof.

The compounds of Formula (I) may be separated into their individual diastereoisomers by, for example, fractional crystallization from a suitable solvent, for example, methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration. Vibrational circular dichroism (VCD) may also be used to determine the absolute stereochemistry. Alternatively, any stereoisomer or isomers of the compounds of Formula (I) may be obtained by stereospecific synthesis using optically pure starting materials or reagents of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereoisomeric mixture, followed by separation of the individual diastereoisomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

The compounds of Formula (I) which contain olefinic double bonds, unless specified otherwise, they are meant to include both E and Z geometric isomers.

Some of the compounds described herein may exist as tautomers which have different points of attachment of hydrogen accompanied by one or more double bond shifts. For example, a ketone and its enol form are keto-enol tautomers. The individual tautomers as well as mixtures thereof are encompassed by the compounds of Formula (I).

Some of the compounds of Formula (I) described herein may exist as atropisomers when the rotational energy barrier around a single bond is sufficiently high to prevent free rotation at a given temperature, thus allowing isolation of individual conformers with distinct properties. The individual atropisomers as well as mixtures thereof are encompassed with compounds of Formula (I) of the present disclosure. When resolved, individual atropisomers can be designated by established conventions such as those specified by the International Union of Pure Applied Chemistry (IUPAC) 2013 Recommendations.

In the compounds of Formula (I), the atoms may exhibit their natural isotopic abundances, or one or more of the atoms may be artificially enriched in a particular isotope having the same atomic number, but an atomic mass or mass number different from the atomic mass or mass number predominantly found in nature. The present disclosure as described and claimed herein is meant to include all suitable isotopic variations of the compounds of Formula (I) and embodiments thereof. For example, different isotopic forms of hydrogen (H) include protium (¹H) and deuterium (²H, also denoted herein as D). Protium is the predominant hydrogen isotope found in nature. Enriching for deuterium may afford certain therapeutic advantages, such as increasing in vivo half-life or reducing dosage requirements or may provide a compound useful as a standard for characterization of biological samples. Isotopically-enriched compounds can be prepared without undue experimentation by conventional techniques well known to those skilled in the art or by processes analogous to those described in the Schemes and Examples herein using appropriate isotopically-enriched reagents and/or intermediates.

The term “pharmaceutically acceptable salts” refers to salts prepared from pharmaceutically acceptable non-toxic bases or acids. When a compound of Formula (I) is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Preferred are the ammonium, calcium, magnesium, potassium and sodium salts. Salts prepared from pharmaceutically acceptable organic non-toxic bases include salts of primary, secondary, and tertiary amines derived from both naturally occurring and synthetic sources. Pharmaceutically acceptable organic non-toxic bases from which salts can be formed include, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, dicyclohexylamine, lysine, methylglucamine, morpholine, piperazine, piperidine, polyamine resins, procaine, purines, theobromine, triethylamine, trimethylamine, tripropylamine, tromethamine and the like.

When a compound of Formula (I) is basic, its corresponding salt can be conveniently prepared from pharmaceutically acceptable non-toxic inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids. If a compound of Formula (I) simultaneously contains acidic and basic groups in the molecule, the disclosure also includes, in addition to the salt forms mentioned, inner salts or betaines (zwitterions). Salts can be obtained from the compounds of Formula (I) by customary methods which are known to the person skilled in the art, for example, by combination with an organic or inorganic acid or base in a solvent or dispersant, or by anion exchange or cation exchange from other salts. The present disclosure also includes all salts of the compounds of Formula (I) which, owing to low physiological compatibility, are not directly suitable for use in pharmaceuticals but which can be used, for example, as intermediates for chemical reactions or for the preparation of pharmaceutically acceptable salts.

Furthermore, the compounds of Formula (I) may exist in amorphous form and/or one or more crystalline forms, and as such all amorphous and crystalline forms and mixtures thereof of the compounds of Formula (I), including the Examples, are intended to be included within the scope of the present disclosure. In addition, some of the compounds of Formula (I) may form solvates with water (i.e., a hydrate) or common organic solvents such as but not limited to ethyl acetate. Such solvates and hydrates, particularly the pharmaceutically acceptable solvates and hydrates, of the instant compounds are likewise encompassed within the scope of this disclosure, along with un-solvated and anhydrous forms.

Any pharmaceutically acceptable pro-drug modification of a compound of Formula (I) which results in conversion in vivo to a compound within the scope of this disclosure is also within the scope of this disclosure.

The present disclosure also relates to processes for the preparation of the compounds of the disclosure which are described in the following and by which the compounds of the disclosure are obtainable.

The terms “therapeutically effective (or efficacious) amount” and similar descriptions such as “an amount efficacious for treatment” or “an effective dose” are intended to mean that amount of a compound of Formula (I) that will elicit the biological or medical response of a tissue, a system, animal or human that is being sought by a researcher, veterinarian, medical doctor or other clinician. In a preferred embodiment, the term “therapeutically effective amount” means an amount of a compound of Formula (I) that alleviates at least one clinical symptom in a human patient. The terms “prophylactically effective (or efficacious) amount” and similar descriptions such as “an amount efficacious for prevention” are intended to mean that amount of a compound of Formula (I) that will prevent or reduce the risk of occurrence of the biological or medical event that is sought to be prevented in a tissue, a system, animal or human by a researcher, veterinarian, medical doctor or other clinician.

### Dosages of the Compounds of Formula (I)

The dosage regimen utilizing a compound of Formula (I) is selected in accordance with a variety of factors including type, species, age, weight, sex and medical condition of the patient; the severity of the condition to be treated; the potency of the compound chosen to be administered; the route of administration; and the renal and hepatic function of the patient. A consideration of these factors is well within the purview of the ordinarily skilled clinician for the purpose of determining the therapeutically effective or prophylactically effective dosage amount needed to prevent, counter, or arrest the progress of the condition. It is understood that a specific daily dosage amount can simultaneously be both a therapeutically effective amount, e.g., for treatment of an oncological condition, and a prophylactically effective amount, e.g., for prevention of an oncological condition.

While individual needs vary, determination of optimal ranges of effective amounts of the compounds of Formula (I) is within the skill of the art. For administration to a human in, for example, the curative or prophylactic treatment of the conditions and disorders identified herein, the typical dosages of the compounds of Formula (I) can be about 0.05 mg/kg/day to about 50 mg/kg/day, or at least 0.05 mg/kg, or at least 0.08 mg/kg, or at least 0.1 mg/kg, or at least 0.2 mg/kg, or at least 0.3 mg/kg, or at least 0.4 mg/kg, or at least 0.5 mg/kg, and any amount therebetween, to about 50 mg/kg or less, or about 40 mg/kg or less, or about 30 mg/kg or less, or about 20 mg/kg or less, or about 10 mg/kg or less and any amount therebetween, which can be, for example, about 2.5 mg/day (0.5 mg/kg×5 kg) to about 5000 mg/day (50 mg/kg×100 kg). For example, dosages of the compounds can be about 0.1 mg/kg/day to about 50 mg/kg/day, or about 0.05 mg/kg/day to about 10 mg/kg/day, or about 0.05 mg/kg/day to about 5 mg/kg/day, or about 0.05 mg/kg/day to about 3 mg/kg/day, or about 0.07 mg/kg/day to about 3 mg/kg/day, or about 0.09 mg/kg/day to about 3 mg/kg/day, or about 0.05 mg/kg/day to about 0.1 mg/kg/day, or about 0.1 mg/kg/day to about 1 mg/kg/day, or about 1 mg/kg/day to about 10 mg/kg/day, or about 1 mg/kg/day to about 5 mg/kg/day, or about 1 mg/kg/day to about 3 mg/kg/day, or about 3 mg/day to about 500 mg/day, or about 5 mg/day to about 250 mg/day, or about 10 mg/day to about 100 mg/day, or about 3 mg/day to about 10 mg/day, or about 100 mg/day to about 250 mg/day. Such doses may be administered in a single dose or may be divided into multiple doses.

### Pharmaceutical Compositions

The compounds of Formula (I) and their pharmaceutically acceptable salts can be administered to animals, preferably to mammals, and in particular to humans, as pharmaceuticals by themselves, in mixtures with one another or in the form of pharmaceutical compositions. The term “subject” or “patient” includes animals, preferably mammals and especially humans, who use the instant active agents for the prevention or treatment of a medical condition. Administering of the drug to the subject includes both self-administration and administration to the patient by another person. The subject may be in need of, or desire, treatment for an existing disease or medical condition, or may be in need of or desire prophylactic treatment to prevent or reduce the risk of occurrence of said disease or medical condition. As used herein, a subject “in need” of treatment of an existing condition or of prophylactic treatment encompasses both a determination of need by a medical professional as well as the desire of a patient for such treatment.

The present disclosure therefore also provides the compounds of the disclosure and their pharmaceutically acceptable salts for use as pharmaceuticals, their use for modulating the activity of mutant KRAS proteins and in particular their use in the therapy and prophylaxis of the below-mentioned diseases or disorders as well as their use for preparing medicaments for these purposes. In certain embodiments, the compounds of the disclosure and their pharmaceutically acceptable salts inhibit the KRAS G12D protein.

Furthermore, the present disclosure provides pharmaceutical compositions which comprise as active component an effective dose of at least one compound of Formula (I) and/or a pharmaceutically acceptable salt thereof and a customary pharmaceutically acceptable carrier, i.e., one or more pharmaceutically acceptable carrier substances and/or additives.

Thus, the present disclosure provides, for example, said compound and its pharmaceutically acceptable salts for use as pharmaceutical compositions which comprise as active component an effective dose of at least one compound of Formula (I) and/or a pharmaceutically acceptable salt thereof and a customary pharmaceutically acceptable carrier, and the uses of said compound and/or a pharmaceutically acceptable salt thereof in the therapy or prophylaxis of the below-mentioned diseases or disorders, e.g., cancer, as well as their use for preparing medicaments for these purposes.

The pharmaceutical compositions according to the disclosure can be administered orally, for example, in the form of pills, tablets, lacquered tablets, sugar-coated tablets, granules, hard and soft gelatin capsules, aqueous, alcoholic or oily solutions, syrups, emulsions or suspensions, or rectally, for example, in the form of suppositories. Administration can also be carried out parenterally, for example subcutaneously, intramuscularly or intravenously in the form of solutions for injection or infusion.

Other suitable administration forms are, for example, percutaneous or topical administration, for example, in the form of ointments, tinctures, sprays or transdermal therapeutic systems, or, for example, microcapsules, implants or rods. The preferred administration form depends, for example, on the disease to be treated and on its severity.

The amount of active compound of a compound described herein and/or its pharmaceutically acceptable salts in the pharmaceutical composition normally is from 0.01 to 200 mg, or from 0.1 to 200 mg, or from 1 to 200 mg, per dose, but depending on the type of the pharmaceutical composition, it can also be higher. In some embodiments, the amount of active compound of a compound of Formula (I) and/or its pharmaceutically acceptable salts in the pharmaceutical composition is from 0.01 to 10 mg per dose. The pharmaceutical compositions usually comprise 0.5 to 90 percent by weight of at least one compound of Formula (I) and/or its pharmaceutically acceptable salts. The preparation of the pharmaceutical compositions can be carried out in a manner known per se. For this purpose, one or more compounds of Formula (I) and/or their pharmaceutically acceptable salts, together with one or more solid or liquid pharmaceutical carrier substances and/or additives (or auxiliary substances) and, if desired, in combination with other pharmaceutically active compounds having therapeutic or prophylactic action, are brought into a suitable administration form or dosage form which can then be used as a pharmaceutical in human or veterinary medicine.

For the production of pills, tablets, sugar-coated tablets and hard gelatin capsules, it is possible to use, for example, lactose, starch, for example, maize starch, or starch derivatives, talc, stearic acid or its salts, etc. Carriers for soft gelatin capsules and suppositories are, for example, fats, waxes, semisolid and liquid polyols, natural or hardened oils, etc. Suitable carriers for the preparation of solutions, for example, of solutions for injection, or of emulsions or syrups are, for example, water, physiologically acceptable sodium chloride solution, alcohols such as ethanol, glycerol, polyols, sucrose, invert sugar, glucose, mannitol, vegetable oils, etc. It is also possible to lyophilize the compounds of Formula (I) and their pharmaceutically acceptable salts and to use the resulting lyophilisates, for example, for preparing preparations for injection or infusion. Suitable carriers for microcapsules, implants or rods are, for example, copolymers of glycolic acid and lactic acid.

Besides the active compounds and carriers, the pharmaceutical compositions can also contain customary additives, for example, fillers, disintegrants, binders, lubricants, wetting agents, stabilizers, emulsifiers, dispersants, preservatives, sweeteners, colorants, flavorings, aromatizers, thickeners, diluents, buffer substances, solvents, solubilizers, agents for achieving a depot effect, salts for altering the osmotic pressure, coating agents and/or antioxidants.

### Methods of Using the Compounds of Formula (I)

The present application provides a method of inhibiting RAS-mediated cell signaling comprising contacting a cell with a compound of Formula (I) or a pharmaceutically acceptable salt thereof. Inhibition of RAS-mediated signal transduction can be assessed and demonstrated by a wide variety of ways known in the art. Non-limiting examples include (a) a decrease in GTPase activity of RAS; (b) a decrease in GTP binding affinity or an increase in GDP binding affinity; (c) an increase in K_{off} of GTP or a decrease in K_{off} of GDP; (d) a decrease in the levels of signaling transduction molecules downstream in the RAS pathway, such as a decrease in pMEK, pERK, or pAKT levels; and/or (e) a decrease in binding of RAS complex to downstream signaling molecules including but not limited to Raf. Kits and commercially available assays can be utilized for determining one or more of the above.

The present application also provides methods of using the compounds of the disclosure (or their pharmaceutically acceptable salts) or pharmaceutical compositions containing such compounds to treat disease conditions, including but not limited to, conditions implicated by mutant KRAS proteins (e.g., cancer), and in some embodiments the KRAS G12D mutant.

In some embodiments, a method for treatment of cancer is provided, the method comprising administering a therapeutically effective amount a compound of the disclosure (or a pharmaceutically acceptable salt thereof) or any of the foregoing pharmaceutical compositions comprising such a compound to a subject in need of such treatment. In some embodiments, the cancer is mediated by a KRAS mutation, e.g., the KRAS G12D mutation. In various embodiments, the cancer is pancreatic cancer, colorectal cancer or lung cancer. In some embodiments, the cancer is gall bladder cancer, thyroid cancer, or bile duct cancer.

In some embodiments the present disclosure provides a method of treating a disorder in a subject in need thereof, wherein said method comprises determining if the subject has a KRAS mutation (e.g., KRAS GT2D mutation) and if the subject is determined to have the KRAS mutation, then administering to the subject a therapeutically effective amount of a compound of the disclosure or a pharmaceutically acceptable salt thereof.

The disclosed compounds inhibit anchorage-independent cell growth and therefore have the potential to inhibit tumor metastasis. Accordingly, another embodiment of the present disclosure provides a method for inhibiting tumor metastasis, the method comprising administering an effective amount a compound of Formula (I).

KRAS, HRAS or NRAS mutations have also been identified in hematological malignancies (e.g., cancers that affect blood, bone marrow and/or lymph nodes). Accordingly, certain embodiments are directed to administration of the compounds of the disclosure (e.g., in the form of a pharmaceutical composition) to a subject in need of treatment of a hematological malignancy. Such malignancies include, but are not limited to leukemias and lymphomas. For example, the presently disclosed compounds can be used for treatment of diseases such as acute lymphoblastic leukemia (ALL), acute myelogenous leukemia (AML), chronic lymphocytic leukemia (CLL), small lymphocytic lymphoma (SLL), chronic myelogenous leukemia (CML), acute monocytic leukemia (AMoL) and/or other leukemias. In other embodiments, the compounds are useful for treatment of lymphomas such as Hodgkins lymphoma or non-Hodgkins lymphoma. In various embodiments, the compounds are useful for treatment of plasma cell malignancies such as multiple myeloma, mantle cell lymphoma, and Waldenstrom's macroglubunemia.

Determining whether a tumor or cancer comprises a KRAS mutation (e.g., the KRAS G12D mutation) can be undertaken by assessing the nucleotide sequence encoding the KRAS protein, by assessing the amino acid sequence of the KRAS protein, or by assessing the characteristics of a putative KRAS mutant protein. The sequences of wild-type human KRAS are known in the art.

Methods for detecting a mutation in a KRAS nucleotide sequence are also known by those of skill in the art. These methods include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct sequencing, primer extension reactions, electrophoresis, oligonucleotide ligation assays, hybridization assays, TaqMan assays, SNP genotyping assays, high resolution melting assays and microarray analyses. In some embodiments, samples are evaluated for KRAS mutations (e.g., the KRAS G12D mutation) by real-time PCR. In real-time PCR, fluorescent probes specific for the KRAS mutation are used. When a mutation is present, the probe binds and fluorescence is detected. In some embodiments, the KRAS mutation is identified using a direct sequencing method of specific regions (e.g., exon 2 and/or exon 3) in the KRAS gene.

Methods for detecting a mutation in a KRAS (e.g., the KRAS G12D mutation) are known by those of skill in the art. These methods include, but are not limited to, detection of a KRAS mutant using a binding agent (e.g., an antibody) specific for the mutant protein, protein electrophoresis and Western blotting, and direct peptide sequencing.

A number of tissue samples can be assessed for determining whether a tumor or cancer comprises a KRAS mutation (e.g., the KRAS G12D mutation). In some embodiments, the sample is taken from a subject having a tumor or cancer. In some embodiments, the sample is a fresh tumor/cancer sample. In some embodiments, the sample is a frozen tumor/cancer sample. In some embodiments, the sample is a formalin-fixed paraffin-embedded sample. In some embodiments, the sample is a circulating tumor cell (CTC) sample. In some embodiments, the sample is processed to a cell lysate. In some embodiments, the sample is processed to DNA or RNA.

The present application also provides a method of treating a hyperproliferative disorder comprising administering a therapeutically effective amount of a compound of the disclosure, or a pharmaceutically acceptable salt thereof to a subject in need thereof. In some embodiments, said method relates to the treatment of a subject who suffers from a cancer such as acute myeloid leukemia, cancer in adolescents, adrenocortical carcinoma childhood, AIDS-related cancers (e.g., Lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, breast cancer, bronchial tumors, Burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, Ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, Hodgkin lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer; multiple endocrine neoplasia syndromes, multiple myeloma/plasma cell neoplasm, mycosis fungoides, myelodysplasia syndromes, myelodysplastic/myeloproliferative neoplasms, multiple myeloma, Merkel cell carcinoma, malignant mesothelioma, malignant fibrous histiocytoma of bone and osteosarcoma, nasal cavity and paranasal sinus cancer, nasopharyngeal cancer, neuroblastoma, Non-Hodgkin lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer; small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or viral-induced cancer. In some embodiments, said method relates to the treatment of a non-cancerous hyperproliferative disorder such as benign hyperplasia of the skin (e.g., psoriasis), restenosis, or prostate (e.g., benign prostatic hypertrophy (BPH)).

In some embodiments, the methods for treatment are directed to treating lung cancers, and the methods comprise administering a therapeutically effective amount of the compounds of Formula (I) (or pharmaceutical composition comprising such compounds) to a subject in need thereof. In certain embodiments, the lung cancer is a non-small cell lung carcinoma (NSCLC), for example, adenocarcinoma, squamous-cell lung carcinoma or large-cell lung carcinoma. In some embodiments, the lung cancer is a small cell lung carcinoma. Other lung cancers which the compounds of Formula (I) may provide therapeutic benefit for include, but are not limited to, glandular tumors, carcinoid tumors and undifferentiated carcinomas.

The present disclosure also provides methods of modulating a mutant KRAS protein activity (e.g., activity resulting from the KRAS G12D mutation) by contacting the protein with an effective amount of a compound of the disclosure. Modulation can be inhibiting or activating protein activity. In some embodiments, the present disclosure provides methods of inhibiting protein activity by contacting the mutant KRAS protein (e.g., KRAS G12D mutant) with an effective amount of a compound of the disclosure in solution. In some embodiments, the present disclosure provides methods of inhibiting the mutant KRAS protein activity by contacting a cell, tissue, or organ that expresses the protein of interest. In some embodiments, the disclosure provides methods of inhibiting protein activity in subjects including, but not limited to, rodents and mammals (e.g., humans) by administering into the subjects an effective amount of a compound of the disclosure.

### Combination Therapies

One or more additional pharmacologically active agents may be administered in combination with a compound of Formula (I) (or a pharmaceutically acceptable salt thereof). An additional active agent (or agents) is intended to mean a pharmaceutically active agent (or agents) that is active in the body, including pro-drugs that convert to pharmaceutically active form after administration, which are different from the compound of Formula (I). The additional active agents also include free-acid, free-base and pharmaceutically acceptable salts of said additional active agents. Generally, any suitable additional active agent or agents, including chemotherapeutic agents or therapeutic antibodies, may be used in any combination with the compound of Formula (I) in a single dosage formulation (e.g., a fixed dose drug combination), or in one or more separate dosage formulations which allows for concurrent or sequential administration of the active agents (co-administration of the separate active agents) to subjects. In addition, the compounds of Formula (I) (or pharmaceutically acceptable salts thereof) can be administered in combination with radiation therapy, hormone therapy, surgery or immunotherapy.

The present application also provides methods for combination therapies in which the additional active agent is known to modulate other pathways, or other components of the same pathway, or even overlapping sets of target enzymes which are used in combination with a compound of Formula (I), or a pharmaceutically acceptable salt thereof. In one embodiment, such therapy includes, but is not limited to, the combination of one or more compounds of Formula (I) with chemotherapeutic agents, immunotherapeutic agents, hormonal and anti-hormonal agents, targeted therapy agents, and anti-angiogenesis agents, to provide a synergistic or additive therapeutic effect. In another embodiment, such therapy includes radiation treatment to provide a synergistic or additive therapeutic effect.

Examples of additional active agents (i.e., additional anti-cancer agents) include chemotherapeutic agents (e.g., cytotoxic agents), immunotherapeutic agents, hormonal and anti-hormonal agents, targeted therapy agents, and anti-angiogenesis agents. Many anti-cancer agents can be classified within one or more of these groups. While certain anti-cancer agents have been categorized within a specific group(s) or subgroup(s) herein, many of these agents can also be listed within one or more other group(s) or subgroup(s), as would be presently understood in the art. It is to be understood that the classification herein of a particular agent into a particular group is not intended to be limiting. Many anti-cancer agents are presently known in the art and can be used in combination with the compounds of the present disclosure.

Further, an agent can be an agonist, antagonist, allosteric modulator, toxin or, more generally, may act to inhibit or stimulate its target (e.g., receptor or enzyme activation or inhibition). For example, suitable for use are one or more agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor “c-met”.

In an embodiment, the additional anti-cancer agent is a chemotherapeutic agent, an immunotherapeutic agent, a hormonal agent, an anti-hormonal agent, a targeted therapy agent, or an anti-angiogenesis agent (or angiogenesis inhibitor). In an embodiment, the additional anti-cancer agent is selected from the group consisting of a chemotherapeutic agent, a mitotic inhibitor, a plant alkaloid, an alkylating agent, an anti-metabolite, a platinum analog, an enzyme, a topoisomerase inhibitor, a retinoid, an aziridine, an antibiotic, a hormonal agent, an anti-hormonal agent, an anti-estrogen, an anti-androgen, an anti-adrenal, an androgen, a targeted therapy agent, an immunotherapeutic agent, a biological response modifier, a cytokine inhibitor, a tumor vaccine, a monoclonal antibody, an immune checkpoint inhibitor, an anti-PD-1 agent, an anti-PD-L1 agent, a colony-stimulating factor, an immunomodulator, an immunomodulatory imide (IMiD), an anti-CTLA4 agent, an anti-LAG1 agent, an anti-LAG3 agent, an anti-ILT4 agent, an anti-OX40 agent, a GITR agonist, a CAR-T cell, a BiTE, a signal transduction inhibitor, a growth factor inhibitor, a tyrosine kinase inhibitor, an EGFR inhibitor, a histone deacetylase (HDAC) inhibitor, a proteasome inhibitor, a cell-cycle inhibitor, an anti-angiogenesis agent, a matrix-metalloproteinase (MMP) inhibitor, a hepatocyte growth factor inhibitor, a TOR inhibitor, a KDR inhibitor, a VEGF inhibitor, a HIF-1α inhibitor, a HIF-2α inhibitor, a fibroblast growth factor (FGF) inhibitor, a RAF inhibitor, a MEK inhibitor, an ERK inhibitor, a PI3K inhibitor, an AKT inhibitor, an MCL-1 inhibitor, a BCL-2 inhibitor, an SHP2 inhibitor, a HER-2 inhibitor, a BRAF-inhibitor, a gene expression modulator, an autophagy inhibitor, an apoptosis inducer, an antiproliferative agent, and a glycolysis inhibitor.

In one embodiment, the additional anti-cancer agent(s) is a chemotherapeutic agent. Non-limiting examples of chemotherapeutic agents include mitotic inhibitors and plant alkaloids, alkylating agents, anti-metabolites, platinum analogs, enzymes, topoisomerase inhibitors, retinoids, aziridines, and antibiotics.

Non-limiting examples of mitotic inhibitors and plant alkaloids include taxanes such as cabazitaxel, docetaxel, larotaxel, ortataxel, paclitaxel, and tesetaxel; demecolcine; epothilone; eribulin; etoposide (VP-16); etoposide phosphate; navelbine; noscapine; teniposide; thaliblastine; vinblastine; vincristine; vindesine; vinflunine; and vinorelbine.

Non-limiting examples of alkylating agents include nitrogen mustards such as chlorambucil, chlornaphazine, cholophosphamide, cytophosphane, estramustine, ifosfamide, mannomustine, mechlorethamine, mechlorethamine oxide hydrochloride, melphalan, novembichin, phenesterine, prednimustine, tris(2-chloroethyl)amine, trofosfamide, and uracil mustard; alkyl sulfonates such as busulfan, improsulfan, and piposulfan; nitrosoureas such as carmustine, chlorozotocin, fotemustine, lomustine, nimustine, ranimustine, streptozotocin, and TA-07; ethylenimines and methylamelamines such as altretamine, thiotepa, triethylenemelamine, triethylenethiophosphaoramide, trietylenephosphoramide, and trimethylolomelamine; ambamustine; bendamustine; dacarbazine; etoglucid; irofulven; mafosfamide; mitobronitol; mitolactol; pipobroman; procarbazine; temozolomide; treosulfan; and triaziquone.

Non-limiting examples of anti-metabolites include folic acid analogues such as aminopterin, denopterin, edatrexate, methotrexate, pteropterin, raltitrexed, and trimetrexate; purine analogs such as 6-mercaptopurine, 6-thioguanine, fludarabine, forodesine, thiamiprine, and thioguanine; pyrimidine analogs such as 5-fluorouracil (5-FU), 6-azauridine, ancitabine, azacytidine, capecitabine, carmofur, cytarabine, decitabine, dideoxyuridine, doxifiuridine, doxifluridine, enocitabine, floxuridine, galocitabine, gemcitabine, and sapacitabine; 3-aminopyridine-2-carboxaldehyde thiosemicarbazone; broxuridine; cladribine; cyclophosphamide; cytarabine; emitefur; hydroxyurea; mercaptopurine; nelarabine; pemetrexed; pentostatin; tegafur; and troxacitabine.

Non-limiting examples of platinum analogs include carboplatin, cisplatin, dicycloplatin, heptaplatin, lobaplatin, nedaplatin, oxaliplatin, satraplatin, and triplatin tetranitrate.

Non-limiting examples of enzymes include asparaginase and pegaspargase.

Non-limiting examples of topoisomerase inhibitors include acridine carboxamide, amonafide, amsacrine, belotecan, elliptinium acetate, exatecan, indolocarbazole, irinotecan, lurtotecan, mitoxantrone, razoxane, rubitecan, SN-38, sobuzoxane, and topotecan.

Non-limiting examples of retinoids include alitretinoin, bexarotene, fenretinide, isotretinoin, liarozole, RII retinamide, and tretinoin.

Non-limiting examples of aziridines include benzodopa, carboquone, meturedopa, and uredopa.

Non-limiting examples of antibiotics include intercalating antibiotics; anthracenediones; anthracycline antibiotics such as aclarubicin, amrubicin, daunomycin, daunorubicin, doxorubicin, epirubicin, idarubicin, menogaril, nogalamycin, pirarubicin, and valrubicin; 6-diazo-5-oxo-L-norleucine; aclacinomysins; actinomycin; authramycin; azaserine; bleomycins; cactinomycin; calicheamicin; carabicin; carminomycin; carzinophilin; chromomycins; dactinomycin; detorubicin; esorubicin; esperamicins; geldanamycin; marcellomycin; mitomycins; mitomycin C; mycophenolic acid; olivomycins; novantrone; peplomycin; porfiromycin; potfiromycin; puromycin; quelamycin; rebeccamycin; rodorubicin; streptonigrin; streptozocin; tanespimycin; tubercidin; ubenimex; zinostatin; zinostatin stimalamer; and zorubicin.

In one embodiment, the additional anti-cancer agent(s) is a hormonal and/or anti-hormonal agent (i.e., hormone therapy). Non-limiting examples of hormonal and anti-hormonal agents include anti-androgens such as abiraterone, apalutamide, bicalutamide, darolutamide, enzalutamide, flutamide, goserelin, leuprolide, and nilutamide; anti-estrogens such as 4-hydroxy tamoxifen, aromatase inhibiting 4(5)-imidazoles, EM-800, fosfestrol, fulvestrant, keoxifene, LY 117018, onapristone, raloxifene, tamoxifen, toremifene, and trioxifene; anti-adrenals such as aminoglutethimide, dexaminoglutethimide, mitotane, and trilostane; androgens such as calusterone, dromostanolone propionate, epitiostanol, mepitiostane, and testolactone; abarelix; anastrozole; cetrorelix; deslorelin; exemestane; fadrozole; finasteride; formestane; histrelin (RL 0903); human chorionic gonadotropin; lanreotide; LDI 200 (Milkhaus); letrozole; leuprorelin; mifepristone; nafarelin; nafoxidine; osaterone; prednisone; thyrotropin alfa; and triptorelin.

In one embodiment, the additional anti-cancer agent(s) is an immunotherapeutic agent (i.e., immunotherapy). Non-limiting examples of immunotherapeutic agents include biological response modifiers, cytokine inhibitors, tumor vaccines, monoclonal antibodies, immune checkpoint inhibitors, colony-stimulating factors, and immunomodulators.

Non-limiting examples of biological response modifiers, including cytokine inhibitors (cytokines) such as interferons and interleukins, include interferon alfa/interferon alpha such as interferon alfa-2, interferon alfa-2a, interferon alfa-2b, interferon alfa-n1, interferon alfa-n3, interferon alfacon-1, peginterferon alfa-2a, peginterferon alfa-2b, and leukocyte alpha interferon; interferon beta such as interferon beta-1a, and interferon beta-1b; interferon gamma such as natural interferon gamma-1a, and interferon gamma-1b; aldesleukin; interleukin-1 beta; interleukin-2; oprelvekin; sonermin; tasonermin; and virulizin.

Non-limiting examples of tumor vaccines include APC 8015, AVICINE, bladder cancer vaccine, cancer vaccine (Biomira), gastrin 17 immunogen, Maruyama vaccine, melanoma lysate vaccine, melanoma oncolysate vaccine (New York Medical College), melanoma vaccine (New York University), melanoma vaccine (Sloan Kettering Institute), TICE® BCG (Bacillus Calmette-Guerin), and viral melanoma cell lysates vaccine (Royal Newcastle Hospital).

Non-limiting examples of monoclonal antibodies include abagovomab, adecatumumab, aflibercept, alemtuzumab, blinatumomab, brentuximab vedotin, CA 125 MAb (Biomira), cancer MAb (Japan Pharmaceutical Development), daclizumab, daratumumab, denosumab, edrecolomab, gemtuzumab zogamicin, HER-2 and Fc MAb (Medarex), ibritumomab tiuxetan, idiotypic 105AD7 MAb (CRC Technology), idiotypic CEA MAb (Trilex), ipilimumab, quavonlimab, vibostolimab, favezelimab, lintuzumab, LYM-1-iodine 131 MAb (Techni clone), mitumomab, moxetumomab, ofatumumab, polymorphic epithelial mucin-yttrium 90 MAb (Antisoma), ranibizumab, rituximab, and trastuzumab.

Non-limiting examples of immune checkpoint inhibitors include anti-PD-1 agents or antibodies such as cemiplimab, nivolumab, and pembrolizumab; anti-PD-L1 agents or antibodies such as atezolizumab, avelumab, and durvalumab; anti-CTLA-4 agents or antibodies such as ipilumumab and quavonlimab; anti-LAG1 agents; anti-LAG3 agents such as favezelimab, and anti-OX40 agents.

Non-limiting examples of colony-stimulating factors include darbepoetin alfa, epoetin alfa, epoetin beta, filgrastim, granulocyte macrophage colony stimulating factor, lenograstim, leridistim, mirimostim, molgramostim, nartograstim, pegfilgrastim, and sargramostim.

Non-limiting examples of additional immunotherapeutic agents include BiTEs, CAR-T cells, GITR agonists, imiquimod, immunomodulatory imides (IMiDs), mismatched double stranded RNA (Ampligen), resiquimod, SRL 172, and thymalfasin.

In one embodiment, the additional anti-cancer agent(s) is a targeted therapy agent (i.e., targeted therapy). Targeted therapy agents include, for example, monoclonal antibodies and small molecule drugs. Non-limiting examples of targeted therapy agents include signal transduction inhibitors, growth factor inhibitors, tyrosine kinase inhibitors, EGFR inhibitors, histone deacetylase (HDAC) inhibitors, proteasome inhibitors, cell-cycle inhibitors, angiogenesis inhibitors, matrix-metalloproteinase (MMP) inhibitors, hepatocyte growth factor inhibitors, TOR inhibitors, KDR inhibitors, VEGF inhibitors, fibroblast growth factors (FGF) inhibitors, MEK inhibitors, ERK inhibitors, PI3K inhibitors, AKT inhibitors, MCL-1 inhibitors, BCL-2 inhibitors, SHP2 inhibitors, HER-2 inhibitors, BRAF-inhibitors, BTK inhibitors (e.g., nemtabrutinib), gene expression modulators, autophagy inhibitors, apoptosis inducers, antiproliferative agents, and glycolysis inhibitors.

Non-limiting examples of signal transduction inhibitors include tyrosine kinase inhibitors, multiple-kinase inhibitors, anlotinib, avapritinib, axitinib, dasatinib, dovitinib, imatinib, lenvatinib, lonidamine, nilotinib, nintedanib, pazopanib, pegvisomant, ponatinib, vandetanib, and EGFR inhibitory agents.

Non-limiting examples of EGFR inhibitory agents include small molecule antagonists of EGFR such as afatinib, brigatinib, erlotinib, gefitinib, lapatinib, and osimertinib; and antibody-based EGFR inhibitors, including any anti-EGFR antibody or antibody fragment that can partially or completely block EGFR activation by its natural ligand. Antibody-based EGFR inhibitory agents may include, for example, those described in Modjtahedi, H., et al., 1993, Br. J. Cancer 67:247-253; Teramoto, T., et al., 1996, Cancer 77:639-645; Goldstein et al, 1995, Clin. Cancer Res. 1: 1311-1318; Huang, S. M., et al., 1999, Cancer Res. 15:59(8): 1935-40; and Yang, X., et al., 1999, Cancer Res. 59: 1236-1243; monoclonal antibody Mab E7.6.3 (Yang, 1999 supra); Mab C225 (ATCC Accession No. HB-8508), or an antibody or antibody fragment having the binding specificity thereof; specific antisense nucleotide or siRNA; afatinib, cetuximab; matuzumab; necitumumab; nimotuzumab; panitumumab; and zalutumumab.

Non-limiting examples of histone deacetylase (HDAC) inhibitors include belinostat, panobinostat, romidepsin, and vorinostat.

Non-limiting examples of proteasome inhibitors include bortezomib, carfilzomib, ixazomib, marizomib (salinosporamide a), and oprozomib.

Non-limiting examples of cell-cycle inhibitors, including CDK inhibitors, include abemaciclib, alvocidib, palbociclib, and ribociclib.

In one embodiment, the additional anti-cancer agent(s) is an anti-angiogenic agent (or angiogenesis inhibitor) including, but not limited to, matrix-metalloproteinase (MMP) inhibitors; VEGF inhibitors; EGFR inhibitors; TOR inhibitors such as everolimus and temsirolimus; PDGFR kinase inhibitory agents such as crenolanib; HIF-1α inhibitors such as PX 478; HIF-2α inhibitors such as belzutifan and the HIF-2α inhibitors described in WO 2015/035223; fibroblast growth factor (FGF) or FGFR inhibitory agents such as B-FGF and RG 13577; hepatocyte growth factor inhibitors; KDR inhibitors; anti-Ang1 and anti-Ang2 agents; anti-Tie2 kinase inhibitory agents; Tek antagonists (US 2003/0162712; U.S. Pat. No. 6,413,932); anti-TWEAK agents (U.S. Pat. No. 6,727,225); ADAM distintegrin domain to antagonize the binding of integrin to its ligands (US 2002/0042368); anti-eph receptor and/or anti-ephrin antibodies or antigen binding regions (U.S. Pat. Nos. 5,981,245; 5,728,813; 5,969,110; 6,596,852; 6,232,447; and 6,057,124); and anti-PDGF-BB antagonists as well as antibodies or antigen binding regions specifically binding to PDGF-BB ligands.

Non-limiting examples of matrix-metalloproteinase (MMP) inhibitors include MMP-2 (matrix-metalloproteinase 2) inhibitors, MMP-9 (matrix-metalloproteinase 9) inhibitors, prinomastat, RO 32-3555, and RS 13-0830. Examples of useful matrix metalloproteinase inhibitors are described, for example, in WO 96/33172, WO 96/27583, EP 1004578, WO 98/07697, WO 98/03516, WO 98/34918, WO 98/34915, WO 98/33768, WO 98/30566, EP 0606046, EP 0931788, WO 90/05719, WO 99/52910, WO 99/52889, WO 99/29667, WO 1999/007675, EP 1786785, EP 1181017, US 2009/0012085, U.S. Pat. Nos. 5,863,949, 5,861,510, and EP 0780386. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13).

Non-limiting examples of VEGF and VEGFR inhibitory agents include bevacizumab, cediranib, CEP 7055, CP 547632, KRN 633, orantinib, pazopanib, pegaptanib, pegaptanib octasodium, semaxanib, sorafenib, sunitinib, VEGF antagonist (Borean, Denmark), and VEGF-TRAP™.

The additional anti-cancer agent(s) may also be another anti-angiogenic agent including, but not limited to, 2-methoxyestradiol, AE 941, alemtuzumab, alpha-D148 Mab (Amgen, US), alphastatin, anecortave acetate, angiocidin, angiogenesis inhibitors, (SUGEN, US), angiostatin, anti-Vn Mab (Crucell, Netherlands), atiprimod, axitinib, AZD 9935, BAY RES 2690 (Bayer, Germany, BC 1 (Genoa Institute of Cancer Research, Italy), beloranib, benefin (Lane Labs, US), cabozantinib, CDP 791 (Celltech Group, UK), chondroitinase AC, cilengitide, combretastatin A4 prodrug, CP 564959 (OSI, US), CV247, CYC 381 (Harvard University, US), E 7820, EHT 0101, endostatin, enzastaurin hydrochloride, ER-68203-00 (IVAX, US), fibrinogen-E fragment, Flk-1 (ImClone Systems, US), forms of FLT 1 (VEGFR 1), FR-111142, GCS-100, GW 2286 (GlaxoSmithKline, UK), IL-8, ilomastat, IM-862, irsogladine, KM-2550 (Kyowa Hakko, Japan), lenalidomide, lenvatinib, MAb alpha5beta3 integrin, second generation (Applied Molecular Evolution, USA and MedImmune, US), MAb VEGF (Xenova, UK), marimastat, maspin (Sosei, Japan), metastatin, motuporamine C, M-PGA, ombrabulin, OXI4503, PI 88, platelet factor 4, PPI 2458, ramucirumab, rBPI 21 and BPI-derived antiangiogenic (XOMA, US), regorafenib, SC-236, SD-7784 (Pfizer, US), SDX 103 (University of California at San Diego, US), SG 292 (Telios, US), SU-0879 (Pfizer, US), TAN-1120, TBC-1635, tesevatinib, tetrathiomolybdate, thalidomide, thrombospondin 1 inhibitor, Tie-2 ligands (Regeneron, US), tissue factor pathway inhibitors (EntreMed, US), tumor necrosis factor-alpha inhibitors, tumstatin, TZ 93, urokinase plasminogen activator inhibitors, vadimezan, vandetanib, vasostatin, vatalanib, VE-cadherin-2 antagonists, xanthorrhizol, XL 784 (Exelixis, US), ziv-aflibercept, and ZD 6126.

In embodiments, the additional anti-cancer agent(s) is an additional active agent that disrupts or inhibits RAS-RAF-ERK or PI3K-AKT-TOR signaling pathways or is a PD-1 and/or PD-L1 antagonist. In embodiments, the additional anti-cancer agent(s) is a RAF inhibitor, EGFR inhibitor, MEK inhibitor, ERK inhibitor, PI3K inhibitor, AKT inhibitor, TOR inhibitor, MCL-1 inhibitor, BCL-2 inhibitor, SHP2 inhibitor, proteasome inhibitor, or immune therapy, including monoclonal antibodies, immunomodulatory imides (IMiDs), anti-PD-1, anti-PDL-1, anti-CTLA4, anti-LAG1, anti-LAG3, and anti-OX40 agents, GITR agonists, CAR-T cells, and BiTEs.

Non-limiting examples of RAF inhibitors include dabrafenib, encorafenib, regorafenib, sorafenib, and vemurafenib.

Non-limiting examples of MEK inhibitors include binimetinib, CI-1040, cobimetinib, PD318088, PD325901, PD334581, PD98059, refametinib, selumetinib, and trametinib.

Non-limiting examples of ERK inhibitors include LY3214996, LTT462, MK-8353, SCH772984, ravoxertinib, ulixertinib, and an ERKi as described in WO 2017/068412.

Non-limiting examples of PI3K inhibitors include 17-hydroxywortmannin analogs (e.g., WO 06/044453); AEZS-136; alpelisib; AS-252424; buparlisib; CAL263; copanlisib; CUDC-907; dactolisib (WO 06/122806); demethoxyviridin; duvelisib; GNE-477; GSK1059615; IC87114; idelalisib; INK1117; LY294002; Palomid 529; paxalisib; perifosine; PI-103; PI-103 hydrochloride; pictilisib (e.g., WO 09/036,082; WO 09/055,730); PIK 90; PWT33597; SF1126; sonolisib; TGI 00-115; TGX-221; XL147; XL-765; wortmannin; and ZSTK474.

Non-limiting examples of AKT inhibitors include Akt-1-1 (inhibits Aktl) (Barnett et al. (2005) Biochem. J., 385 (Pt. 2), 399-408); Akt-1-1,2 (Barnett et al. (2005) Biochem. J. 385 (Pt. 2), 399-408); API-59CJ-Ome (e.g., Jin et al. (2004) Br. J. Cancer 91, 1808-12); 1-H-imidazo[4,5-c]pyridinyl compounds (e.g., WO05011700); indole-3-carbinol and derivatives thereof (e.g., U.S. Pat. No. 6,656,963; Sarkar and Li (2004) J Nutr. 134(12 Suppl), 34935-34985); perifosine, Dasmahapatra et al. (2004) Clin. Cancer Res. 10(15), 5242-52, 2004); phosphatidylinositol ether lipid analogues (e.g., Gills and Dennis (2004) Expert. Opin. Investig. Drugs 13, 787-97); triciribine (Yang et al. (2004) Cancer Res. 64, 4394-9); imidazooxazone compounds including trans-3-amino-1-methyl-3-[4-(3-phenyl-5H-imidazo[1,2-c]pyrido[3,4-e][1,3]oxazin-2-yl)phenyl]-cyclobutanol hydrochloride (WO 2012/137870); afuresertib; capivasertib; MK2206; patasertib, and those disclosed in WO 2011/082270 and WO 2012/177844.

Non-limiting examples of TOR inhibitors include deforolimus; ATP-competitive TORC1/TORC2 inhibitors, including PI-103, PP242, PP30, and Torin 1; TOR inhibitors in FKBP12 enhancer, rapamycins and derivatives thereof, including temsirolimus, everolimus, WO 9409010; rapalogs, e.g. as disclosed in WO 98/02441 and WO 01/14387, e.g. AP23573, AP23464, or AP23841; 40-(2-hydroxyethyl)rapamycin, 40-[3-hydroxy(hydroxymethyl)methylpropanoate]-rapamycin; 40-epi-(tetrazolyl)-rapamycin (also called ABT578); 32-deoxorapamycin; 16-pentynyloxy-32(S)-dihydrorapanycin, and other derivatives disclosed in WO 05/005434; derivatives disclosed in U.S. Pat. No. 5,258,389, WO 94/090101, WO 92/05179, U.S. Pat. Nos. 5,118,677, 5,118,678, 5,100,883, 5,151,413, 5,120,842, WO 93/111130, WO 94/02136, WO 94/02485, WO 95/14023, WO 94/02136, WO 95/16691, WO 96/41807, WO 96/41807 and U.S. Pat. No. 5,256,790; and phosphorus-containing rapamycin derivatives (e.g., WO 05/016252).

Non-limiting examples of MCL-1 inhibitors include AMG-176, MIK665, and 563845.

Non-limiting examples of SHP2 inhibitors include SHP2 inhibitors described in WO 2019/167000 and WO 2020/022323.

Additional non-limiting examples of anti-cancer agents that are suitable for use include 2-ethylhydrazide, 2,2′,2″-trichlorotriethylamine, ABVD, aceglatone, acemannan, aldophosphamide glycoside, alpharadin, amifostine, aminolevulinic acid, anagrelide, ANCER, ancestim, anti-CD22 immunotoxins, antitumorigenic herbs, apaziquone, arglabin, arsenic trioxide, azathioprine, BAM 002 (Novelos), bcl-2 (Genta), bestrabucil, biricodar, bisantrene, bromocriptine, brostallicin, bryostatin, buthionine sulfoximine, calyculin, cell-cycle nonspecific antineoplastic agents, celmoleukin, clodronate, clotrimazole, cytarabine ocfosfate, DA 3030 (Dong-A), defofamine, denileukin diftitox, dexrazoxane, diaziquone, dichloroacetic acid, dilazep, discodermolide, docosanol, doxercalciferol, edelfosine, eflornithine, EL532 (Elan), elfomithine, elsamitrucin, eniluracil, etanidazole, exisulind, ferruginol, folic acid replenisher such as frolinic acid, gacytosine, gallium nitrate, gimeracil/oteracil/tegafur combination (S-1), glycopine, histamine dihydrochloride, HIT diclofenac, HLA-B7 gene therapy (Vical), human fetal alpha fetoprotein, ibandronate, ibandronic acid, ICE chemotherapy regimen, imexon, iobenguane, IT-101 (CRLX101), laniquidar, LC 9018 (Yakult), leflunomide, lentinan, levamisole+fluorouracil, lovastatin, lucanthone, masoprocol, melarsoprol, metoclopramide, miltefosine, miproxifene, mitoguazone, mitozolomide, mopidamol, motexafnn gadolinium, MX6 (Galderma), naloxone+pentazocine, nitracrine, nolatrexed, NSC 631570 octreotide (Ukrain), olaparib, P-30 protein, PAC-1, palifermin, pamidronate, pamidronic acid, pentosan polysulfate sodium, phenamet, picibanil, pixantrone, platinum, podophyllinic acid, porfimer sodium, PSK (Polysaccharide-K), rabbit antithymocyte polyclonal antibody, rasburiembodiment, retinoic acid, rhenium Re 186 etidronate, romurtide, samarium (153 Sm) lexidronam, sizofiran, sodium phenylacetate, sparfosic acid, spirogermanium, strontium-89 chloride, suramin, swainsonine, talaporfin, tariquidar, tazarotene, tegafur-uracil, temoporfin, tenuazonic acid, tetrachlorodecaoxide, thrombopoietin, tin ethyl etiopurpurin, tirapazamine, TLC ELL-12, tositumomab-iodine 131, trifluridine and tipiracil combination, troponin I (Harvard University, US), urethan, valspodar, verteporfin, zoledronic acid, and zosuquidar.

The present disclosure further provides a method for using the compounds of Formula (I) or pharmaceutical compositions provided herein, in combination with radiation therapy to treat cancer. Techniques for administering radiation therapy are known in the art, and these techniques can be used in the combination therapy described herein. The administration of the compound of Formula (I) in this combination therapy can be determined as described herein.

Radiation therapy can be administered through one of several methods, or a combination of methods, including, without limitation, external-beam therapy, internal radiation therapy, implant radiation, stereotactic radiosurgery, systemic radiation therapy, radiotherapy and permanent or temporary interstitial brachy therapy. The term “brachytherapy,” as used herein, refers to radiation therapy delivered by a spatially confined radioactive material inserted into the body at or near a tumor or other proliferative tissue disease site. The term is intended, without limitation, to include exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner of the present disclosure include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, Y-90. Moreover, the radionuclide(s) can be embodied in a gel or radioactive microspheres.

The present disclosure also provides methods for combination therapies in which the additional active agent is known to modulate other pathways, or other components of the same pathway, or even overlapping sets of target enzymes which are used in combination with a compound of Formula (I), or a pharmaceutically acceptable salt thereof. In one embodiment, such therapy includes, but is not limited to, the combination of one or more compounds of Formula (I) with chemotherapeutic agents, immunotherapeutic agents, hormonal therapy agents, therapeutic antibodies, targeted therapy agents, and radiation treatment, to provide a synergistic or additive therapeutic effect.

The compounds of the disclosure can be used in combination with the agents disclosed herein or other suitable agents, depending on the condition being treated. Hence, in some embodiments the one or more compounds of the disclosure will be co-administered with other agents as described above. When used in combination therapy, the compounds described herein are administered with the second agent simultaneously or separately. This administration in combination can include simultaneous administration of the two agents in the same dosage form, simultaneous administration in separate dosage forms, and separate administration. That is, a compound of Formula (I) and any of the agents described above can be formulated together in the same dosage form and administered simultaneously. Alternatively, a compound of Formula (I) and any of the agents described above can be simultaneously administered, wherein both the agents are present in separate formulations. In another alternative, a compound of Formula (I) can be administered just followed by and any of the agents described above, or vice versa. In some embodiments of the separate administration protocol, a compound of Formula (I) and any of the agents described above are administered a few minutes apart, or a few hours apart, or a few days apart.

As one aspect of the present disclosure contemplates the treatment of the disease/conditions with a combination of pharmaceutically active compounds that may be administered separately, the disclosure further relates to combining separate pharmaceutical compositions in kit form. The kit comprises two separate pharmaceutical compositions: a compound of Formula (I), and a second pharmaceutical compound. The kit comprises a container for containing the separate compositions such as a divided bottle or a divided foil packet. Additional examples of containers include syringes, boxes, and bags. In some embodiments, the kit comprises directions for the use of the separate components. The kit form is particularly advantageous when the separate components are preferably administered in different dosage forms (e.g., oral and parenteral), are administered at different dosage intervals, or when titration of the individual components of the combination is desired by the prescribing health care professional.

The present disclosure also provides for the compound of Formula (I), or the pharmaceutically acceptable salt thereof, for use in therapy, or use of the compound of Formula (I), or the pharmaceutically acceptable salt thereof, in therapy. The present disclosure also provides for the compound of Formula (I), or the pharmaceutically acceptable salt thereof, for use in treating cancer, or use of a compound of Formula (I), or the pharmaceutically acceptable salt thereof, for treating cancer. The present disclosure also provides for the compound of Formula (I), or the pharmaceutically acceptable salt thereof, for the preparation of a medicament for the treatment of cancer, or use of the compound of Formula (I), or the pharmaceutically acceptable salt thereof, for the preparation of a medicament for the treatment of cancer. The present disclosure also provides for the compound of Formula (I), or the pharmaceutically acceptable salt thereof, and an additional anti-cancer agent, for use in the treatment of cancer, or use of the compound of Formula (I), or the pharmaceutically acceptable salt thereof, and the additional anti-cancer agent for treating cancer. The disclosure also provides the compound of Formula (I), or the pharmaceutically acceptable salt thereof, and an additional anti-cancer agent, for the preparation of a medicament for the treatment of cancer, or use of the compound of Formula (I), or the pharmaceutically acceptable salt thereof, and the additional anti-cancer agent, for the preparation of a medicament for the treatment of cancer. The present disclosure also provides for a pharmaceutical composition comprising the compound of Formula (I), or the pharmaceutically acceptable salt thereof, for use in the treatment of cancer, or use of the pharmaceutical composition comprising the compound of Formula (I), or the pharmaceutically acceptable salt thereof, for treating cancer. The present disclosure also provides for a pharmaceutical composition comprising the compound of Formula (I), or the pharmaceutically acceptable salt thereof, and an additional anti-cancer agent, for use in the treatment of cancer, or use of the pharmaceutical composition comprising the compound of Formula (I), or the pharmaceutically acceptable salt thereof, and the additional anti-cancer agent, for treating cancer.

### Methods of Preparing the Compounds of the Disclosure

The compounds described herein can be prepared according to the procedures of the following schemes and examples, using appropriate materials and are further exemplified by the following specific examples. The compounds illustrated in the examples are not, however, to be construed as forming the only genus that is considered as the disclosure. The examples further illustrate details for the preparation of the compounds of the present disclosure. Those skilled in the art will readily understand that known variations of the conditions and processes of the following preparative procedures can be used to prepare these compounds. For instance, in some cases, the order of carrying out the steps of reaction schemes may be varied to facilitate the reaction or to avoid unwanted reaction products. These examples are provided for the purpose of further illustration only and are not intended to be limitations on the disclosure. Any intermediates described below may be referred to herein by their number preceded by “Int-.”

Throughout the synthetic schemes and examples, abbreviations and acronyms may be used with the following meanings unless otherwise indicated: Ac=acetyl; AcO=acetate; AcOH=acetic acid; Am=amyl; tAm=tert-amyl; tAmOH=tert-amyl alcohol; aq.=aqueous; atm=atmosphere; BI-DIME=3-(tert-butyl)-4-(2,6-dimethoxyphenyl)-2,3-dihydrobenzo[d][1,3]oxaphospholeBn=benzyl; BnOH=benzyl alcohol; BO=BnO=benzyloxy; Boc=tert-butyloxycarbonyl; Boc₂O=di-tert-butyl decarbonate; BOMCl=((chloromethoxy)methyl)benzene; BOP=benzotriazol-1-yloxytris(dimethylamino)phosphonium hexafluorophosphate; B₂pin₂=bis(pinacolato)diboron; Bu=butyl; tBu=tert-butyl; tBuO=tert-butoxide; conc.=concentrated; cataCXium A=di(1-adamantyl)-n-butylphosphine; cataCXium A Pd G2=chloro[(di(1-adamantyl)-N-butylphosphine)-2-(2-aminobiphenyl)]palladium(II); cataCXium A Pd G3=mesylate[(di(1-adamantyl)-n-butylphosphine)-2-(2′-amino-1,1′-biphenyl)]palladium(II); conc.=concentrated; Cy=cyclohexyl; DAST=(diethylamino)sulfur trifluoride; dba=dibenzylideneacetone; DBU=1,8-diazabicyclo[5.4.0]undec-7-ene; DCE=1,2-dichloroethane; DCM=dichloromethane; DHP=3,4-dihydro-2H-pyran; DIEA=DIPEA=N,N-diisopropylethylamine; DMA=N,N-dimethylacetamide; DMAP=4-(dimethylamino)pyridine; DME=dimethyl ether; DMF=N,N-dimethylformamide; DMSO=dimethylsulfoxide; dppf=1,1′-bis(diphenylphosphino)ferrocene; equiv, eq.=equivalent(s); ESI=electrospray ionization; Et=ethyl; EtOAc=ethyl acetate; EtOH=ethanol; FA=formic acid; Fmoc=fluorenylmethoxycarbonyl, GDP=guanosine diphosphate; glyme=ethylene glycol dimethyl ether; Grubbs-II=(1,3-bis(2,4,6-trimethylphenyl)-2-imidazolidinylidene)dichloro(phenylmethylene)(tricyclohexylphosphine)ruthenium; GTP=guanosine triphosphate; h=hour; HMDS=hexamethydisilazane; HPLC=high pressure liquid chromatography; Int=intermediate; i-Pr=isopropyl; IPA=i-PrOH=isopropyl alcohol; KHMDS=potassium bis(trimethylsilyl)amide; LAH=lithium aluminum hydride; LCMS=liquid chromatography-mass spectrometry; min=minute; LDA=lithium diisopropylamide; LiHMDS=Lithium bis(trimethylsilyl)amide; M=Molar; mCPBA=3-chlorobenzoperoxoic acid=m-chloroperoxybenzoic acid; Me=methyl; MeCN=acetonitrile; MeOH=methanol; MO=methoxy; MOMCl=chloro(methoxy)methane; MPLC=medium pressure liquid chromatography; MS=mass spectrometry; N=Normal; NBS=N-bromosuccinimide; NMP=N-methyl-2-pyrrolidone; NMR=nuclear magnetic resonance; PMB=4-methoxybenzyl; PMBCl=1-(chloromethyl)-4-methoxybenzene; Pet. ether=petroleum ether; Ph=phenyl; phen=phenanthroline; pin=pinacolato; POCl₃=phosphorus(V) oxide chloride; PPTS=pyridiniump-toluenesulfonate; rac=racemic; r.t.=room temperature; RuPhos Pd G4=[dicyclohexyl(2′,6′-diisopropoxy-2-biphenylyl)phosphine-κP](methanesulfonatato-κO)[2′-(methylamino-κN)-2-biphenylyl-κC²]palladium; sat.=saturated; SCN—CO₂Et=ethoxycarbonyl isothiocyanate; SEM=(2-methoxyethyl)trimethylsilane; SEMCl=(2-(chloromethoxy)ethyl)trimethylsilane; SFC=supercritical fluid chromatography; SPhos Pd G3=(2-dicyclohexylphosphino-2′,6′-dimethoxybiphenyl) [2-(2′-amino-1,1′-biphenyl)]palladium(II) methanesulfonate; TEA=Et₃N=triethylamine; TES=triethylsilyl; Tf=trifluoromethanesulfonyl; TfO=trifluoromethanesulfonate; TfOH=trifluoromethanesulfonic acid; Tf₂O=trifluoromethanesulfonic anhydride; TFA=trifluoroacetic acid; THF=tetrahydrofuran; THP=tetrahydropyran; TIPS=triisopropypsilyl; TLC=thin layer chromatography; THF=tetrahydrofuran; TLC=thin layer chromatography; TMS=trimethylsilyl; TMSCN=Trimethylsilyl cyanide; TsOH=p-toluenesulfonic acid=4-methylbenzenesulfonic acid; Tween=polyoxyethylene (20) sorbitan monolaurate; TMP=2,2,6,6-tetramethylpiperidine; VCD=vibrational circular dichroism; v, v/v=volume, volume to volume; w, w/w=weight, weight to weight, XPhos Pd G2=chloro(2-dicyclohexylphosphino-2′,4′,6′-triisopropyl-1,1′-biphenyl)[2-(2′-amino-1,1′-biphenyl)]palladium(II); μm=micrometer.

### EXAMPLES

Concentration refers to the removal of the volatile components at reduced pressure (e.g., by rotary evaporation) unless otherwise noted. All temperatures are in degrees Celsius unless otherwise noted. Mass spectra (MS) were measured by electrospray ion-mass spectroscopy (ESI) in positive ion detection mode and m/z refers to the [M+H]+ ion unless otherwise noted. 1H NMR spectra were recorded at 400-600 MHz at ambient temperature unless otherwise noted. Protons reported as 0.5 H are due to rotameric signals. RP-HPLC refers to reverse-phase HPLC on C18-functionalized preparative or semi-preparative columns with gradient elution using acetonitrile and water modified with trifluoroacetic acid or ammonium hydroxide as eluents and fractions were lyophilized or concentrated by rotary evaporation unless otherwise noted. Purification by column chromatography on silica gel was accomplished using a flash chromatography system (e.g., ISCO® or Biotage®) and commercial pre-packed silica gel columns with elution using the stated solvent systems. Compounds described herein were synthesized as the racemates unless otherwise noted in the experimental procedures and compound tables. Certain products/intermediates in the examples include indication of “Peak 1” and/or “Peak 2”, which refer to the order of elution of the indicated product/intermediate from the chromatography column (e.g., an SFC column) used to isolate the compound under the specified conditions. Thus, for example, Peak 1 refers to the first eluting compound, e.g., first eluting stereoisomer, under the specified conditions.

SFC and HPLC Columns used in the resolution of stereoisomers are summarized in the following Table:

| | Column |
|---|---|
| SFC/HPLC Column | Abbreviation |
| Daicel Chiralcel ® OJ (250 mm × 30 mm, 10 um) | Column A |
| CCO F4 (21 × 250 mm, 5 um) | Column B |
| Daicel Chirapak ® IC (250 mm × 30 mm, 10 um) | Column C |
| Daicel Chiralcel ® OD (250 mm × 30 mm, 10 um) | Column D |
| YMC-IB (250 nm × 30 mm, 10 um) | Column E |

### Intermediate Syntheses

### Intermediate 1: (6-methyl-1-(tetrahydro-2H-pyran-2-yl)-5-(trifluoromethyl)-1H-indazol-4-yl)boronic acid (Int-1)

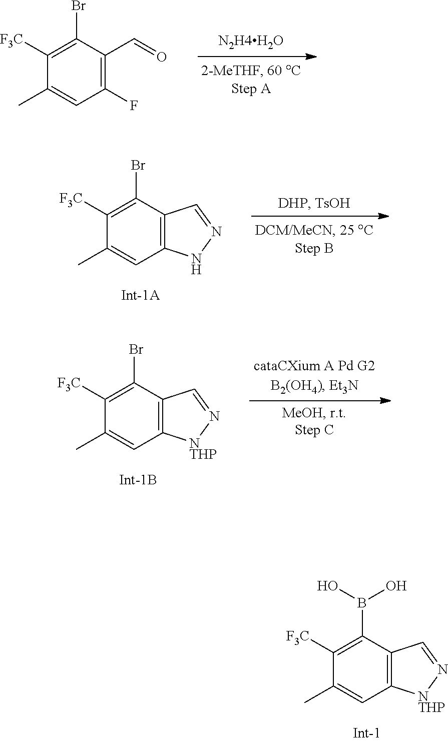

### Step A: 4-bromo-6-methyl-5-(trifluoromethyl)-1H-indazole (Int-1A)

2-bromo-6-fluoro-4-methyl-3-(trifluoromethyl)benzaldehyde (100 g, 0.351 mol) was dissolved in 2-MeTHF (800 mL). To this mixture was added N₂H₄H₂O (53.7 g, 1.05 mol) at 25° C. The mixture was heated and stirred for 2 h at 60° C. The product mixture was quenched with water (400 mL) and extracted with EtOAc (200 mL×2). The combined organic layers were washed with brine (200 mL) and dried over Na₂SO₄. The dried solution was filtered and the filtrate was concentrated in vacuo to give the residue. This reaction was repeated in 2 additional batches using the above conditions. The three batches of reactions were combined. The residue obtained was triturated with DCM (100 mL) at 15° C. for 2 h. The solid was collected by filtration to afford 4-bromo-6-methyl-5-(trifluoromethyl)-1H-indazole (Int-1A). ¹H NMR (400 MHz, CDCl₃) δ 10.61-10.20 (m, 1H), 8.20 (d, J=0.8 Hz, 1H), 7.34 (d, J=0.6 Hz, 1H), 2.67-2.63 (m, 3H).

### Step B: 4-bromo-6-methyl-1-(tetrahydro-2H-pyran-2-yl)-5-(trifluoromethyl)-1H-indazole (Int-1B)

4-bromo-6-methyl-5-(trifluoromethyl)-1H-indazole (Int-1A) (60.0 g, 0.215 mol) was dissolved in DCM (240 mL) and MeCN (240 mL). DHP (21.7 g, 0.258 mol) and TsOH·H₂O (8.18 g, 0.043 mol) were added to the mixture at 20° C. The reaction mixture was stirred for 12 h at 25° C. Water (200 mL) was added to the mixture. The resulting mixture was extracted with DCM (200 mL×2). The combined organic layers were washed with brine (200 mL) and dried over Na₂SO₄. The dried solution was filtered and the filtrate was concentrated under reduced pressure. The residue obtained was purified by column chromatography (SiO₂, Petroleum ether:Ethyl acetate=1:0 to 0:1) to afford 4-bromo-6-methyl-1-(tetrahydro-2H-pyran-2-yl)-5-(trifluoromethyl)-1H-indazole (Int-1B). ¹H NMR (400 MHz, CDCl₃-d) δ 8.12 (s, 1H), 7.44 (s, 1H), 5.69 (dd, J=3, 9 Hz, 1H), 4.09-3.94 (m, 1H), 3.81-3.69 (m, 1H), 2.69-2.63 (m, 3H), 2.56-2.43 (m, 1H), 2.19-2.14 (m, 1H), 2.12-2.04 (m, 1H), 1.87-1.73 (m, 2H), 1.71-1.63 (m, 1H).

### Step C: (6-methyl-1-(tetrahydro-2H-pyran-2-yl)-5-(trifluoromethyl)-1H-indazol-4-yl)boronic acid (Int-1)

To a solution of 4-bromo-6-methyl-1-(tetrahydro-2H-pyran-2-yl)-5-(trifluoromethyl)-1H-indazole (Int-1B) (1.2 g, 3.30 mmol) in MeOH (15 mL) was added tetrahydroxydiboron (1.19 g, 13.22 mmol), TEA (1.38 mL, 9.91 mmol), and cataCXium A Pd G2 (0.110 g, 0.165 mmol). The mixture was stirred at r.t. for 16 h under N₂ atmosphere. The mixture was concentrated in vacuo. The residue was purified by flash silica gel chromatography (0-20% EE (EtOAc:EtOH=3:1)/petroleum ether) to give (6-methyl-1-(tetrahydro-2H-pyran-2-yl)-5-(trifluoromethyl)-1H-indazol-4-yl)boronic acid (Int-1). MS (ESI) [M+H]⁺: m/z 329.

The compound in the table below was synthesized using a similar procedure as described in the synthesis of Int-1 by making the appropriate substitutions for starting material, intermediates, and/or reagents. Appropriate substitutions are available commercially, synthesized as described in the literature, synthesized using methods available to those skilled in the art, or synthesized as described herein.

| | Starting | | | [M + H]⁺ |
|---|---|---|---|---|
| Int. | Material | Structure | Compound Name | Found |
| Int-2 | 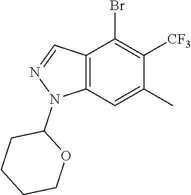 [SMILES: Cc1cc2c(cnn2C2CCCCO2)c(Br)c1C(F)(F)F] Int-1E | 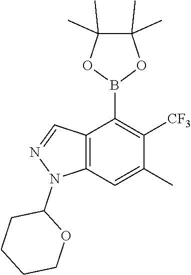 [SMILES: Cc1cc2c(cnn2C2CCCCO2)c(B2OC(C)(C)C(C)(C)O2)c1C(F)(F)F] | 6-methyl-1-(tetrahydro-2H- pyran-2-yl)-4-(4,4,5,5- tetramethyl-1,3,2- dioxaborolan-2-yl)-5- (trifluoromethyl)-1H-indazole | 411 |

### Intermediate 3: (6-chloro-5-(difluoromethyl)-1-(tetrahydro-2H-pyran-2-yl)-1H-indazol-4-yl)boronic acid (Int-3)

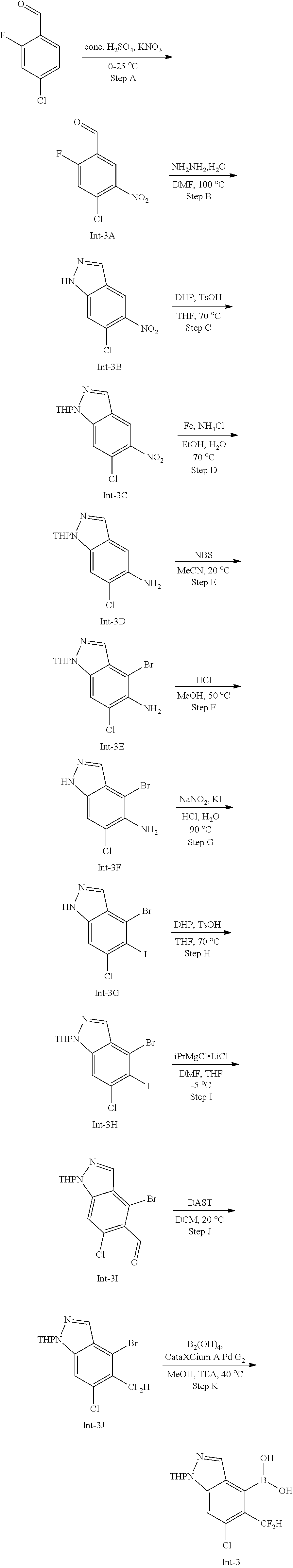

### Step A: 4-chloro-2-fluoro-5-nitrobenzaldehyde (Int-3A)

To a solution of 4-chloro-2-fluorobenzaldehyde (58 g, 370 mmol) in conc. H₂SO₄ (500 mL) was added potassium nitrate (47 g, 470 mmol) at 0° C. Then the reaction was stirred at 25° C. for 1 h. The reaction mixture was quenched with ice water (2 L), filtered, and the solid was washed with water (2×500 mL), dried in vacuo to give 4-chloro-2-fluoro-5-nitrobenzaldehyde (Int-3A). ¹H NMR (400 MHz, CDCl₃) δ 10.32 (s, 1H), 8.47 (d, J=6.6 Hz, 1H), 7.48 (d, J=9.2 Hz, 1H).

### Step B: 6-chloro-5-nitro-1H-indazole (Int-3B)

To a solution of 4-chloro-2-fluoro-5-nitrobenzaldehyde (Int-3A) (67.0 g, 329 mmol) in DMF (1.00 L) were added hydrazine hydrate (161 mL, 2.82 mol) at 25° C. under N₂ atmosphere. The reaction mixture was stirred at 100° C. for 15 h. The reaction mixture was cooled and quenched with ice water (2 L). The mixture was filtered and the filtered cake was washed with water (2×300 mL) and dried in vacuo to give 6-chloro-5-nitro-1H-indazole (Int-3B). MS (ESI) [M+H]+: m/z 198. ¹H NMR (400 MHz, DMSO-d₆) δ 8.44 (s, 1H), 8.25 (s, 1H), 7.70 (s, 1H).

### Step C: 6-chloro-5-nitro-1-(tetrahydro-2H-pyran-2-yl)-1H-indazole (Int-3C)

To a solution of 6-chloro-5-nitro-1H-indazole (Int-3B) (92 g, 0.47 mol) in THF (1.0 L) were added 3,4-dihydro-2H-pyran (85 mL, 0.93 mol) and p-toluenesulfonic acid (8.02 g, 47 mmol) at 25° C. under N₂ atmosphere. The reaction mixture was stirred at 70° C. for 5 h. The reaction mixture was cooled and evaporated under reduced pressure. The product was purified by flash silica gel chromatography (20% ethyl acetate in petroleum ether) to give 6-chloro-5-nitro-1-(tetrahydro-2H-pyran-2-yl)-1H-indazole (Int-3C). MS (ESI) [M+H]⁺: m/z 282. ¹H NMR (400 MHz, CDCl₃) δ 8.38 (s, 1H), 8.16 (s, 1H), 7.81 (s, 1H), 5.73 (dd, J=8.9, 2.5 Hz, 1H), 3.99-4.04 (m, 1H), 3.76-3.81 (m, 1H), 2.46-2.50 (m, 1H), 2.10-2.18 (m, 2H), 1.71-1.77 (m, 3H).

### Step D: 6-chloro-1-(tetrahydro-2H-pyran-2-yl)-1H-indazol-5-amine (Int-3D)

To a solution of 6-chloro-5-nitro-1-(tetrahydro-2H-pyran-2-yl)-1H-indazole (Int-3C) (38.6 g, 137 mmol) in EtOH (1.00 L) and water (200 mL) were added ammonium chloride (22.0 g, 411 mmol) and iron dust (38.3 g, 685 mmol) while stirring at 25° C. under N₂ atmosphere. The reaction mixture was stirred at 70° C. for 15 h. The reaction mixture was cooled, diluted with EtOAc (200 mL), filtered, and the filtrate was concentrated in vacuo. The residue was then dissolved in EtOAc (3×300 mL), washed with brine (100 mL), dried over Na₂SO₄, filtered, and the filtrate was concentrated under reduced pressure. The product was purified by flash silica gel chromatography (15% EtOAc in petroleum ether) to give 6-chloro-1-(tetrahydro-2H-pyran-2-yl)-1H-indazol-5-amine (Int-3D). MS (ESI) [M+H]+: m/z 252. ¹H NMR (400 MHz, CDCl₃) δ 7.82 (d, J=0.8 Hz, 1H), 7.60 (s, 1H), 7.04 (s, 1H), 5.61 (dd, J=9.4, 2.7 Hz, 1H), 4.02-4.07 (m, 1H), 3.89-4.01 (m, 2H), 3.71-3.77 (m, 1H), 2.49-2.56 (m, 1H), 2.06-2.17 (m, 2H), 1.68-1.79 (m, 3H).

### Step E: 4-bromo-6-chloro-1-(tetrahydro-2H-pyran-2-yl)-1H-indazol-5-amine (Int-3E)

To a solution of 6-chloro-1-(tetrahydro-2H-pyran-2-yl)-1H-indazol-5-amine (Int-3D) (66.6 g, 265 mmol) in MeCN (660 mL) was added NBS (56.5 g, 318 mmol) at 20° C. under N₂ atmosphere. The reaction mixture was stirred at 20° C. for 3 h. The reaction mixture was quenched with water (200 mL), diluted with EtOAc (200 mL), filtered, and concentrated. The residue was extracted with EtOAc (2×200 mL), washed with brine (100 mL), dried over Na₂SO₄, filtered, and the filtrate was concentrated under reduced pressure. The product was purified by flash silica gel chromatography (10% EtOAc in petroleum ether) to give 4-bromo-6-chloro-1-(tetrahydro-2H-pyran-2-yl)-1H-indazol-5-amine (Int-3E). MS (ESI) [M+H]⁺: m/z 330, 332.

### Step F: 4-bromo-6-chloro-1H-indazol-5-amine (Int-3F)

To a solution of 4-bromo-6-chloro-1-(tetrahydro-2H-pyran-2-yl)-1H-indazol-5-amine (Int-3E) (30 g, 90 mmol) was added 4N HCl in MeOH (300 mL). The reaction was stirred at 50° C. for 2 h. The mixture was cooled and evaporated under reduced pressure to give 4-bromo-6-chloro-1H-indazol-5-amine (Int-3F) isolated as the HCl salt. MS (ESI) [M+H]⁺: m/z 246, 248.

### Step G: 4-bromo-6-chloro-5-iodo-1H-indazole (Int-3G)

To a solution of 4-bromo-6-chloro-1H-indazol-5-amine (Int-3F), HCl (10 g, 35 mmol) in 6M aq. HCl (100 mL) was added a solution of sodium nitrite (2.9 g, 42.4 mmol) in water (20 mL) dropwise at −5° C. and stirred for 5 min. Then, a solution of KI (23 g, 140 mmol) in water (100 mL) was added dropwise to the reaction mixture at −5° C. The reaction mixture was stirred at 90° C. for 1 h. The reaction was cooled, quenched with ice water (150 mL) and sat. aq. Na₂SO₃ (100 mL), and then basified with sat. aq. NaHCO₃ (200 mL) to pH 8. The aqueous layer was extracted with EtOAc (2×200 mL) and the organic layer was dried over Na₂SO₄, filtered, and the solvent was evaporated under reduced pressure to give 4-bromo-6-chloro-5-iodo-1H-indazole (Int-3G). MS (ESI) [M+H]⁺: m/z 357, 359.

### Step H: 4-bromo-6-chloro-5-iodo-1-(tetrahydro-2H-pyran-2-yl)-1H-indazole (Int-3H)

To a solution of 4-bromo-6-chloro-5-iodo-1H-indazole (Int-3G) (32 g, 90 mmol) in THF (300 mL) were added 4-methylbenzenesulfonic acid (1.5 g, 9.0 mmol) and 3,4-dihydro-2H-pyran (16 mL, 180 mmol) at 20° C. under N₂ atmosphere. The reaction mixture was stirred at 70° C. for 3 h. The mixture was cooled and concentrated under reduced pressure. The product was purified by flash silica gel chromatography (10% THF in petroleum ether) to give 4-bromo-6-chloro-5-iodo-1-(tetrahydro-2H-pyran-2-yl)-1H-indazole (Int-3H). MS (ESI) [M+H]⁺: m/z 441, 443. ¹H NMR (400 MHz, CDCl₃) δ 7.93 (s, 1H), 7.79 (d, J=0.7 Hz, 1H), 5.65 (dd, J=8.8, 2.7 Hz, 1H), 3.96-4.00 (m, 1H), 3.71-3.77 (m, 1H), 2.42-2.50 (m, 1H), 2.07-2.16 (m, 2H), 1.69-1.79 (m, 3H).

### Step I: 4-bromo-6-chloro-1-(tetrahydro-2H-pyran-2-yl)-1H-indazole-5-carbaldehyde (Int-3I)

To a solution of 4-bromo-6-chloro-5-iodo-1-(tetrahydro-2H-pyran-2-yl)-1H-indazole (Int-3H) (8.0 g, 18.12 mmol) in THF (60 mL) and DMF (30 mL, 390 mmol) was added isopropylmagnesium chloride lithium chloride complex (15.3 mL, 20 mmol, 1.3M in THF) at −5° C. The mixture was stirred at −5° C. for 10 min. Three batches were prepared in this fashion. The combined reactions were quenched with ice water (100 mL) and extracted with EtOAc (3×100 mL). The organic layer was washed with brine (50 mL), dried over sodium sulfate, filtered, and concentrated in vacuo. The residue was purified by flash silica gel chromatography (0 to 15% THF in petroleum ether), followed by recrystallization with EtOAc/Pet. ether (~10:1) to give 4-bromo-6-chloro-1-(tetrahydro-2H-pyran-2-yl)-1H-indazole-5-carbaldehyde (Int-3I). MS (ESI) [M+H]⁺: m/z 343, 345. ¹H NMR (400 MHz, CDCl₃) δ 10.51 (s, 1H), 8.19 (s, 1H), 7.71 (s, 1H), 5.70 (dd, J=8.9, 2.6 Hz, 1H), 3.99-4.03 (m, 1H), 3.74-3.80 (m, 1H), 2.42-2.49 (m, 1H), 2.08-2.16 (m, 2H), 1.70-1.79 (m, 3H).

### Step J: 4-bromo-6-chloro-5-(difluoromethyl)-1-(tetrahydro-2H-pyran-2-yl)-1H-indazole (Int-3J)

To a solution of 4-bromo-6-chloro-1-(tetrahydro-2H-pyran-2-yl)-1H-indazole-5-carbaldehyde (Int-3I) (8.0 g, 23 mmol) in DCM (100 mL) was added DAST (25 mL, 190 mmol) under N₂ atmosphere. The reaction was stirred at 20° C. for 16 h. The reaction was diluted with EtOAc (200 mL) and quenched with sat. aq. NaHCO₃ (150 mL). The organic layer was washed with brine (50 mL), dried, and concentrated in vacuo. The residue was purified by flash silica gel chromatography (20% THF in petroleum ether) to give 4-bromo-6-chloro-5-(difluoromethyl)-1-(tetrahydro-2H-pyran-2-yl)-1H-indazole (Int-3J). MS (ESI) [M+H]⁺: m/z 365, 367. ¹H NMR (400 MHz, MeOD) δ 8.17 (s, 1H), 7.94 (s, 1H), 7.24-7.57 (m, 1H), 5.84 (dd, J=2.62, 9.18 Hz, 1H), 3.95 (br s, 1H), 3.84 (br d, J=13.23 Hz, 1H), 2.45 (br d, J=11.44 Hz, 1H), 2.02-2.18 (m, 2H), 1.64-1.89 (m, 3H).

### Step K: (6-chloro-5-(difluoromethyl)-1-(tetrahydro-2H-pyran-2-yl)-1H-indazol-4-yl)boronic acid (Int-3)

To a solution of 4-bromo-6-chloro-5-(difluoromethyl)-1-(tetrahydro-2H-pyran-2-yl)-1H-indazole (Int-3J) (2.0 g, 5.5 mmol) in MeOH (40 mL) was added tetrahydroxydiboron (2.0 g, 22 mmol), TEA (2.3 mL, 16 mmol), and CataXCium A Pd G2 (0.18 g, 0.27 mmol). The mixture was stirred at 25° C. for 1 h under N₂ atmosphere. The mixture was concentrated under reduced pressure. The residue was acidified with aqueous HCl (0.5 M) to pH ~6 at 0° C., and the mixture was extracted with EtOAc (50 mL). The organic layer was washed with brine (3×3 mL), dried over Na₂SO₄, filtered, and concentrated in vacuo. The residue was purified by flash silica gel chromatography (0 to 50% EtOH/EtOAc) to give (6-chloro-5-(difluoromethyl)-1-(tetrahydro-2H-pyran-2-yl)-1H-indazol-4-yl)boronic acid (Int-3). MS (ESI) [M+H]⁺: m/z 331. ¹H NMR (400 MHz, CDCl₃) δ 8.07-8.17 (m, 1H), 7.70-7.76 (m, 1H), 7.26-7.29 (m, 1H), 5.66-5.76 (m, 1H), 3.70-3.85 (m, 2H), 2.07 (d, J=4.53 Hz, 2H), 1.66-1.86 (m, 4H).

The compound in the table below was synthesized using a similar procedure as described in the synthesis of Int-3 by making the appropriate substitutions for starting material, intermediates, and/or reagents. Appropriate substitutions are available commercially, synthesized as described in the literature, synthesized using methods available to those skilled in the art, or synthesized as described herein.

| | Starting | | | [M + H]⁺ |
|---|---|---|---|---|
| Int. | Material | Structure | Compound Name | Found |
| Int-4 | 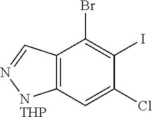 [SMILES: Clc1cc2[nH]ncc2c(Br)c1I ; "THP"] | 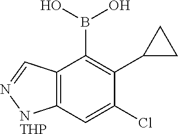 [SMILES: OB(O)c1c(C2CC2)c(Cl)cc2[nH]ncc12 ; "THP"] | (6-chloro-5-cyclopropyl-1- (tetrahydro-2H-pyran-2-yl)- 1H-indazol-4-yl)boronic acid | 321 |
| | Int-3H | | | |

### Intermediate 45: (5-(1-fluorocyclopropyl)-1-(tetrahydro-2H-pyran-2-yl)-6-(trifluoromethyl)-1H-indazol-4-yl)boronic acid (Int-45)

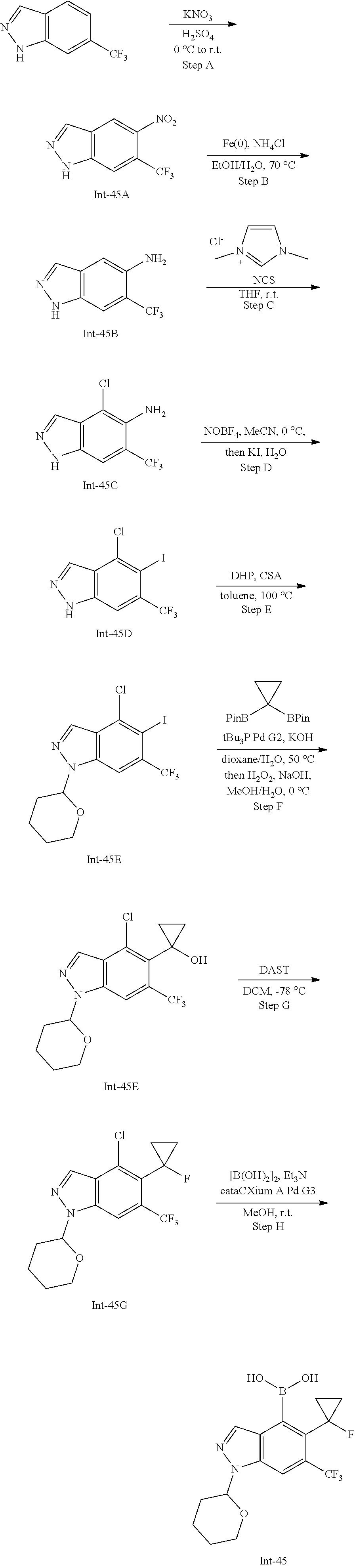

### Step A: 5-nitro-6-(trifluoromethyl)-1H-indazole (Int-45A)

To a solution of 6-(trifluoromethyl)-1H-indazole (7.44 g, 40 mmol) in H₂SO₄ (100 mL, conc. aq.) at 0° C., was slowly added KNO₃ (4.45 g, 44 mmol). The reaction mixture was warmed up to room temperature. After stirring for 3 h at room temperature, it was poured into crushed ice. The precipitated solid was collected by filtration and washed with water to afford 5-nitro-6-(trifluoromethyl)-1H-indazole (Int-45A). MS (ESI) [M+H]⁺: m/z 232.

### Step B: 6-(trifluoromethyl)-1H-indazol-5-amine (Int-45B)

A mixture of 5-nitro-6-(trifluoromethyl)-1H-indazole (Int-45A) (8.91 g, 38.5 mmol), iron (10.8 g, 193 mmol), and NH₄Cl (10.3 g, 193 mmol) in EtOH (120 mL) and water (20 mL) was vigorously stirred at 70° C. for 1 h. The mixture was diluted with EtOAc (~200 mL), filtered by glass fiber membrane filter, and washed with EtOAc. The filtrate was concentrated under reduced pressure and sat. aq. NaHCO₃ and EtOAc were added. The layers were separated, and the organic phase was washed with brine, dried over Na₂SO₄, filtered, and concentrated to give 6-(trifluoromethyl)-1H-indazol-5-amine (Int-45B). MS (ESI) [M+H]⁺: m/z 202.

### Step C: 4-chloro-6-(trifluoromethyl)-1H-indazol-5-amine (Int-45C)

To a solution of 6-(trifluoromethyl)-1H-indazol-5-amine (Int-45B) (616 mg, 3.06 mmol) in THF (15 mL) was added 1,3-dimethylimidazolium chloride (40 mg, 0.302 mmol) and NCS (430 mg, 3.22 mmol). After stirring overnight at room temperature, sat. aq. NaHCO₃ and EtOAc were added to the reaction mixture. The layers were separated, and the organic phase was washed with brine, dried over Na₂SO₄, filtered, and concentrated. The residue was purified by silica gel chromatography (0 to 100% EtOAc in hexanes) to give 4-chloro-6-(trifluoromethyl)-1H-indazol-5-amine (Int-45C). MS (ESI) [M+H]⁺: m/z 236.

### Step D: 4-chloro-5-iodo-6-(trifluoromethyl)-1H-indazole (Int-45D)

To a solution of 4-chloro-6-(trifluoromethyl)-1H-indazol-5-amine (Int-45C) (634 mg, 2.69 mmol) in MeCN (20 mL) was added nitrosyl tetrafluoroborate (380 mg, 3.25 mmol) at 0° C. The mixture was stirred at 0° C. for 10 min before a solution of KI (5.0 g, 30 mmol) in water (10 mL) was added with vigorous stirring. After stirring for 10 min, EtOAc and water were added, and the layers were separated. The organic phase was washed with the mixture of sat. aq. NaHCO₃ and Na₂S₂O₃ solution, dried over Na₂SO₄, and concentrated to give 4-chloro-5-iodo-6-(trifluoromethyl)-1H-indazole (Int-45D). MS (ESI) [M+H]⁺: m/z 347.

### Step E: 4-chloro-5-iodo-1-(tetrahydro-2H-pyran-2-yl)-6-(trifluoromethyl)-1H-indazole (Int-45E)

A mixture of 4-chloro-5-iodo-6-(trifluoromethyl)-1H-indazole (Int-45D) (920 mg), (1R)-(−)-camphor-10-sulfonic acid (60 mg, 0.26 mmol), and 3,4-dihydro-2H-pyran (0.72 mL, 8.0 mmol) in toluene (20 mL) was stirred at 100° C. for 2.5 h. The mixture was cooled to room temperature, and sat. aq. NaHCO₃ was added. The layers were separated, and the organic phase was washed with brine, dried over Na₂SO₄, filtered, and concentrated. The residue was purified by silica gel chromatography (0 to 100% EtOAc in hexanes) to give 4-chloro-5-iodo-1-(tetrahydro-2H-pyran-2-yl)-6-(trifluoromethyl)-1H-indazole (Int-45E). MS (ESI) [M+H]⁺: m/z 431.

### Step F: 1-(4-chloro-1-(tetrahydro-2H-pyran-2-yl)-6-(trifluoromethyl)-1H-indazol-5-yl)cyclopropan-1-ol (Int-45F)

KOH (1.25 mL, 2.50 mmol, 2N in H₂O) was added to a solution of (4-chloro-5-iodo-1-(tetrahydro-2H-pyran-2-yl)-6-(trifluoromethyl)-1H-indazole (Int-45E) (430 mg, 0.999 mmol), 2,2′-cyclopropylidenebis(4,4,5,5-tetramethyl-1,3,2-dioxaborolane) (740 mg, 2.52 mmol), and chloro[(tri-tert-butylphosphine)-2-(2-aminobiphenyl)]palladium(II) (100 mg, 0.195 mmol) in 1,4-dioxane (10 mL). The mixture was stirred at 50° C. for 3 h. The mixture was cooled to 0° C., and MeOH (1 mL) and NaOH (5 mL, 2N in H₂O) were added. Hydrogen peroxide (1.2 mL, 30 wt % in H₂O) was added slowly at 0° C. and stirred for 10 min. After 10 min, MeOH (2 mL) was added and stirred at 0° C. for 30 min. The reaction was quenched with H₃PO₄ (10% aq.) and CHCl₃, and the layers were separated. The organic phase was washed with brine, dried over Na₂SO₄, filtered, and concentrated. The residue was purified by silica gel chromatography (0 to 100% EtOAc in hexanes) to afford 1-(4-chloro-1-(tetrahydro-2H-pyran-2-yl)-6-(trifluoromethyl)-1H-indazol-5-yl)cyclopropan-1-ol (Int-45F). MS (ESI) [M+H]⁺: m/z 361.

### Step G: 4-chloro-5-(1-fluorocyclopropyl)-1-(tetrahydro-2H-pyran-2-yl)-6-(trifluoromethyl)-1H-indazole (Int-45G)

Diethylaminosulfur trifluoride (0.035 mL, 0.265 mmol) was added to a solution of 1-(4-chloro-1-(tetrahydro-2H-pyran-2-yl)-6-(trifluoromethyl)-1H-indazol-5-yl)cyclopropan-1-ol (Int-45F) (43 mg, 0.119 mmol) in DCM (3 mL) at −78° C. After stirring at −78° C. for 30 min, sat. aq. NaHCO₃ and EtOAc were added to the reaction mixture. The layers were separated, and the organic phase was washed with brine, dried over Na₂SO₄, filtered, and concentrated under reduced pressure. The residue was purified by silica gel chromatography (0 to 100% EtOAc in hexanes) to give 4-chloro-5-(1-fluorocyclopropyl)-1-(tetrahydro-2H-pyran-2-yl)-6-(trifluoromethyl)-1H-indazole (Int-45G). MS (ESI) [M+H]⁺: m/z 363.

### Step H: (5-(1-fluorocyclopropyl)-1-(tetrahydro-2H-pyran-2-yl)-6-(trifluoromethyl)-1H-indazol-4-yl)boronic acid (Int-45)

The mixture of 4-chloro-5-(1-fluorocyclopropyl)-1-(tetrahydro-2H-pyran-2-yl)-6-(trifluoromethyl)-1H-indazole (Int-45G) (34 mg, 0.094 mmol), tetrahydroxydiboron (40 mg, 0.446 mmol), cataCXium A Pd G3 (6 mg, 0.008 mmol) and Et₃N (0.080 mL, 0.574 mmol) in MeOH (1 mL) was stirred at room temperature for 60 hours. EtOAc (30 mL), CHCl₃ (1 mL), and H₃PO₄ (10% aq.) were added to the reaction mixture. The layers were separated, and the organic phase was washed with brine, dried over Na₂SO₄, filtered, and concentrated under reduced pressure. The residue was purified by silica gel chromatography (0 to 100% EtOAc in hexanes) to give (5-(1-fluorocyclopropyl)-1-(tetrahydro-2H-pyran-2-yl)-6-(trifluoromethyl)-1H-indazol-4-yl)boronic acid (Int-45). MS (ESI) [M+H]⁺: m/z 373.

### Intermediate 46: (5-cyclopropyl-1-(tetrahydro-2H-pyran-2-yl)-6-(trifluoromethyl)-1H-indazol-4-yl)boronic acid (Int-46)

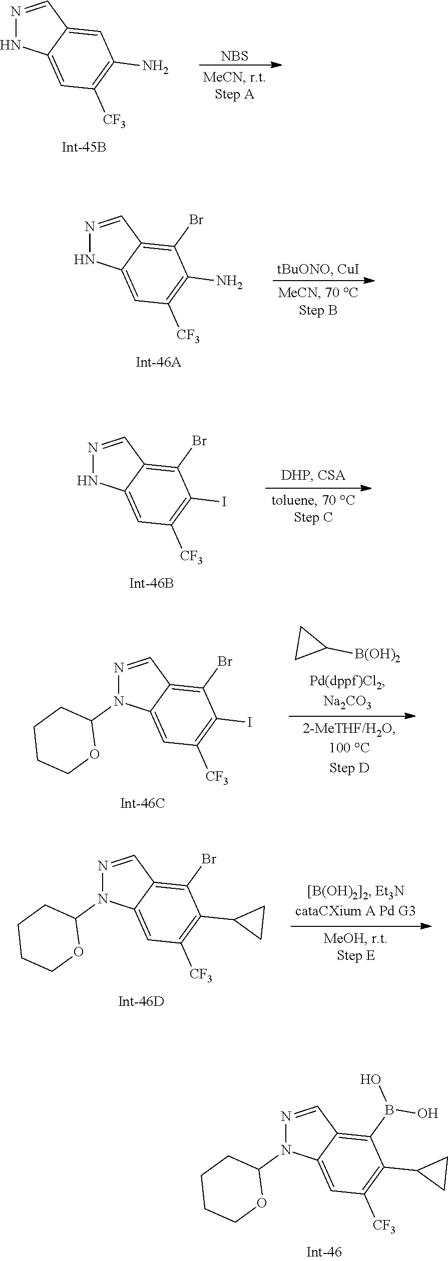

### Step A: 4-bromo-6-(trifluoromethyl)-1H-indazol-5-amine (Int-46A)

NBS (880 mg, 5.0 mmol) was added to a stirred solution of 6-(trifluoromethyl)-1H-indazol-5-amine (Int-45B) (1.0 g, 5.0 mmol) in MeCN (25 mL) at room temperature. After 10 min, the mixture was partitioned between EtOAc and sat. aq. NaHCO₃. The organic phase was washed with brine, dried over Na₂SO₄, and concentrated. The crude residue was purified via silica gel chromatography (0 to 20% EtOAc in hexane) to give 4-bromo-6-(trifluoromethyl)-1H-indazol-5-amine (Int-46A). MS (ESI): m/z (M+H)⁺ 280.

### Step B: 4-bromo-5-iodo-6-(trifluoromethyl)-1H-indazole (Int-46B)

Copper iodide (612 mg, 3.21 mmol) and tert-butyl nitrite (0.395 ml, 3.32 mmol) were added to a stirred solution of 4-bromo-6-(trifluoromethyl)-1H-indazol-5-amine (Int-46A) (300 mg, 1.07 mmol) in MeCN (25 mL) at room temperature. The mixture was warmed to 70° C. and stirred for 30 min. After cooling, the mixture was partitioned between EtOAc and sat. aq. NaHCO₃. The organic phase was washed with brine, dried over Na₂SO₄, and concentrated. The crude residue was purified via silica gel chromatography (0 to 20% EtOAc in hexane) to give 4-bromo-5-iodo-6-(trifluoromethyl)-1H-indazole (Int-46B). MS (ESI): m/z (M+H)⁺ 391.

### Step C: 4-bromo-5-iodo-1-(tetrahydro-2H-pyran-2-yl)-6-(trifluoromethyl)-1H-indazole (Int-46C)

A mixture of 4-bromo-5-iodo-6-(trifluoromethyl)-1H-indazole (Int-46B) (318 mg, 0.812 mmol), 3,4-dihydro-2H-pyran (0.15 mL, 1.61 mmol), (1R)-(−)-camphor-10-sulfonic acid (24 mg, 0.107 mmol) in THF (10 mL) was stirred at 70° C. for 3 h. After cooling to room temperature, the mixture was partitioned between EtOAc and aqueous sat. aq. NaHCO₃. The organic phase was washed with brine, dried over Na₂SO₄, filtered, and concentrated under reduced pressure. The residue was purified via silica gel chromatography (0 to 10% EtOAc in hexane) to give 4-bromo-5-iodo-1-(tetrahydro-2H-pyran-2-yl)-6-(trifluoromethyl)-1H-indazole (Int-46C). MS (ESI): m/z (M+H)⁺ 475, 477.

### Step D: 4-bromo-5-cyclopropyl-1-(tetrahydro-2H-pyran-2-yl)-6-(trifluoromethyl)-1H-indazole (Int-46D)

A mixture of 4-bromo-5-iodo-1-(tetrahydro-2H-pyran-2-yl)-6-(trifluoromethyl)-1H-indazole (Int-46C) (170 mg, 0.36 mmol), cyclopropylboronic acid (31 mg, 0.36 mmol), [1,1′-bis(diphenylphosphino)ferrocene]dichloropalladium(II) (52 mg, 0.071 mmol), sodium carbonate (1.07 mL, 1.07 mmol, 1 M in H₂O) in 2-methyltetrahydrofuran (3.6 mL) was evacuated and backfilled with N₂ (3×). The mixture was stirred at 100° C. under N₂ for 8 h. Additional cyclopropylboronic acid (15 mg, 0.179 mmol) and [1,1′-bis(diphenylphosphino)ferrocene]dichloropalladium(II) (26 mg, 0.036 mmol) were added to the mixture and stirred at 100° C. for 2 h. After cooling to room temperature, the mixture was partitioned between EtOAc and water. The organic phase was washed with brine, dried over Na₂SO₄, filtered, and concentrated under reduced pressure. The residue was purified via silica gel chromatography (0 to 10% EtOAc in hexane) to give 4-bromo-5-cyclopropyl-1-(tetrahydro-2H-pyran-2-yl)-6-(trifluoromethyl)-1H-indazole (Int-46D). MS (ESI): m/z (M+H)⁺ 389, 391.

### Step E: (5-cyclopropyl-1-(tetrahydro-2H-pyran-2-yl)-6-(trifluoromethyl)-1H-indazol-4-yl)boronic acid (Int-46)

CataCXium A Pd G3 (12 mg, 0.017 mmol) was added to a stirred mixture of 4-bromo-5-cyclopropyl-1-(tetrahydro-2H-pyran-2-yl)-6-(trifluoromethyl)-1H-indazole (Int-46D) (66 mg, 0.170 mmol), triethylamine (0.095 mL, 0.680 mmol), and tetrahydroxydiboron (31 mg, 0.340 mmol) in MeOH (3.4 mL) at room temperature. The reaction was stirred at room temperature for 1 h. The mixture was filtered through a pad of CELITE®, washed with MeOH, and the filtrate was concentrated. The residue was purified via silica gel chromatography (10 to 50% EtOAc in hexanes) to give (5-cyclopropyl-1-(tetrahydro-2H-pyran-2-yl)-6-(trifluoromethyl)-1H-indazol-4-yl)boronic acid (Int-46). MS (ESI): m/z (M+H)⁺ 355.

### Intermediate 5: (5-(difluoromethyl)-6-methyl-1-(tetrahydro-2H-pyran-2-yl)-1H-indazol-4-yl)boronic acid (Int-5)

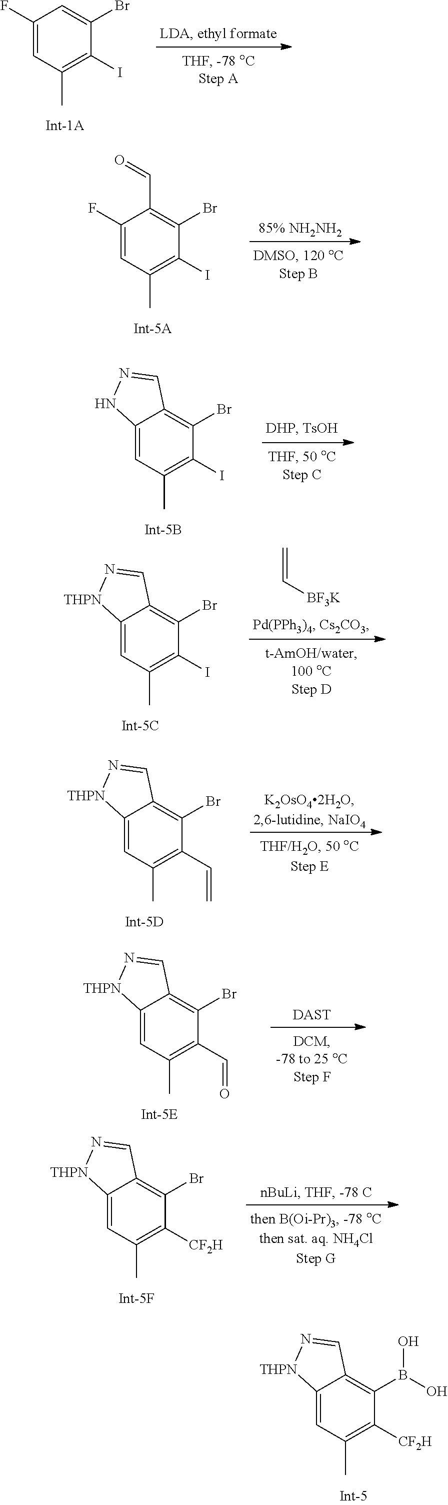

### Step A: 2-bromo-6-fluoro-3-iodo-4-methylbenzaldehyde (Int-5A)

To a solution of diisopropylamine (15.0 mL, 106 mmol) in THF (45 ml) was added n-butyllithium (40 mL, 100 mmol, 2.5 M in hexanes) at −78° C., and the mixture was stirred at −78° C. for 30 min to give LDA. To a solution of 1-bromo-5-fluoro-2-iodo-3-methylbenzene (Int-1A) (24 g, 76 mmol) in THF (45 ml) was added LDA (91 mL, 91 mmol, 1 M in THF) at −78° C. under N₂ atmosphere. The mixture was stirred at −78° C. for 0.5 h, charged with ethyl formate (6.21 g, 84 mmol), and the resulting mixture was stirred at −78° C. for 15 min. The mixture was slowly poured into sat. aq. NH₄Cl (200 mL) and extracted with EtOAc (3×400 mL). The organic layer was dried over sodium sulfate, filtered, and the solvent was concentrated in vacuo. The residue was purified by silica gel chromatography (5% EtOAc in petroleum ether) to give 2-bromo-6-fluoro-3-iodo-4-methylbenzaldehyde (Int-5A). ¹H NMR (400 MHz, CDCl₃) δ 10.16-10.02 (m, 1H), 7.05 (d, J=11.0 Hz, 1H), 2.56 (s, 3H).

### Step B: 4-bromo-5-iodo-6-methyl-1H-indazole (Int-5B)

To a solution of 2-bromo-6-fluoro-3-iodo-4-methylbenzaldehyde (Int-5A) (18.7 g, 54.5 mmol) in DMSO (200 mL) was added hydrazine (19.8 mL, 327 mmol, 85% aqueous solution) at 20° C. under N₂ atmosphere. The reaction mixture was stirred at 120° C. for 12 h. The reaction mixture was poured into ice water (50 mL) and extracted with EtOAc (3×400 mL). The organic layer was washed with sat. aq. NaCl (2×50 mL), dried over sodium sulfate, filtered, and the solvent was concentrated in vacuo to give 4-bromo-5-iodo-6-methyl-1H-indazole (Int-5B), which was used directly in the next step without further purification. MS (ESI): m/z (M+H)⁺ 337, 339.

### Step C: 4-bromo-5-iodo-6-methyl-1-(tetrahydro-2H-pyran-2-yl)-1H-indazole (Int-5C)

To a solution of 4-bromo-5-iodo-6-methyl-1H-indazole (Int-5B) (15.5 g, 46.0 mmol) in THF (200 mL) was added 4-methylbenzenesulfonic acid (1.58 g, 9.20 mmol) and DHP (8.41 mL, 92 mmol) at 20° C., and the mixture was stirred at 50° C. for 12 h. The mixture was concentrated in vacuo, and the residue was purified by silica gel chromatography (0 to 15% EtOAc in petroleum ether). The collected solid was diluted with MeOH (50 mL), and the resulting mixture was stirred for 5 min and then filtered. The solid was collected and dried in vacuum to afford 4-bromo-5-iodo-6-methyl-1-(tetrahydro-2H-pyran-2-yl)-1H-indazole (Int-5C). MS (ESI): m/z (M+H)⁺ 421, 423. ¹H NMR (400 MHz, CDCl₃) δ 7.84 (s, 1H), 7.44-7.35 (m, 1H), 5.58 (dd, J=2.7, 9.1 Hz, 1H), 4.00-3.85 (m, 1H), 3.73-3.59 (m, 1H), 2.67-2.58 (m, 3H), 2.52-2.36 (m, 1H), 2.16-1.93 (m, 2H), 1.76-1.51 (m, 3H).

### Step D: 4-bromo-6-methyl-1-(tetrahydro-2H-pyran-2-yl)-5-vinyl-1H-indazole (Int-5D)

To a solution of 4-bromo-5-iodo-6-methyl-1-(tetrahydro-2H-pyran-2-yl)-1H-indazole (Int-5C) (6.0 g, 14.3 mmol) in t-amyl alcohol (60 mL) and water (20 mL) was added potassium vinyltrifluoroborate (2.48 g, 18.5 mmol), Cs₂CO₃ (13.93 g, 42.7 mmol), and Pd(PPh₃)₄ (0.823 g, 0.712 mmol) at 25° C. The mixture was stirred at 100° C. for 16 h under N₂. The reaction mixture was quenched with H₂O (20 mL) and extracted with EtOAc (200×3 mL). The organic layer was dried over Na₂SO₄, filtered, and the solvent was concentrated in vacuo. The residue was purified by silica gel chromatography (0 to 15% EtOAc in petroleum ether) to give 4-bromo-6-methyl-1-(tetrahydro-2H-pyran-2-yl)-5-vinyl-1H-indazole (Int-5D). MS (ESI): m/z (M+H)⁺ 321, 323.

### Step E: 4-bromo-6-methyl-1-(tetrahydro-2H-pyran-2-yl)-1H-indazole-5-carbaldehyde (Int-5E)

To a solution of 4-bromo-6-methyl-1-(tetrahydro-2H-pyran-2-yl)-5-vinyl-1H-indazole (Int-5D) (4.5 g, 12.9 mmol) in THF (50 mL) and water (50 mL) was added sodium periodate (11.0 g, 51.6 mmol), 2,6-lutidine (2.76 g, 25.8 mmol), and potassium osmate(VI) dihydrate (0.475 g, 1.29 mmol) at 25° C., and the mixture was stirred at 50° C. for 2 h. The reaction mixture was quenched with sat. aq. Na₂SO₃ (20 mL), and the mixture was extracted with EtOAc (3×200 mL). The combined organic layer was washed with brine (20 mL), dried over anhydrous Na₂SO₄, filtered, and concentrated in vacuo. The residue was purified by silica gel chromatography (petroleum ether/EtOAc=5/1) to give 4-bromo-6-methyl-1-(tetrahydro-2H-pyran-2-yl)-1H-indazole-5-carbaldehyde (Int-5E). MS (ESI): m/z (M+H)+323, 325. ¹H NMR (400 MHz, CDCl₃) δ 10.62-10.44 (m, 1H), 8.09 (s, 1H), 7.30 (s, 1H), 5.62 (dd, J=2.8, 9.1 Hz, 1H), 4.02-3.85 (m, 1H), 3.69 (ddd, J=3.0, 10.0, 11.6 Hz, 1H), 2.66 (d, J=0.7 Hz, 3H), 2.51-2.34 (m, 1H), 2.17-1.92 (m, 2H), 1.78-1.56 (m, 3H).

### Step F: 4-bromo-5-(difluoromethyl)-6-methyl-1-(tetrahydro-2H-pyran-2-yl)-1H-indazole (Int-5F)

To a solution of 4-bromo-6-methyl-1-(tetrahydro-2H-pyran-2-yl)-1H-indazole-5-carbaldehyde (Int-5E) (3.0 g, 9.28 mmol) in DCM (30 mL) was added DAST (6.13 mL, 46.4 mmol) at −78° C. under N₂ atmosphere, and the mixture was stirred at 25° C. for 12 h. The reaction mixture was added dropwise to aq. NaHCO₃ (50 mL), and the mixture was extracted with EtOAc (3×120 mL). The combined organic phase was dried over anhydrous Na₂SO₄, filtered, and concentrated in vacuo. The residue was purified by silica gel chromatography (petroleum ether/EtOAc=5/1) to give 4-bromo-5-(difluoromethyl)-6-methyl-1-(tetrahydro-2H-pyran-2-yl)-1H-indazole (Int-5F). MS (ESI): m/z (M+H)⁺ 345, 347. ¹H NMR (400 MHz, CDCl₃) δ 7.96 (s, 1H), 7.45-7.06 (m, 2H), 5.61 (dd, J=2.8, 9.1 Hz, 1H), 3.97-3.88 (m, 1H), 3.72-3.60 (m, 1H), 2.62 (s, 3H), 2.51-2.38 (m, 1H), 2.14-1.95 (m, 2H), 1.77-1.54 (m, 3H).

### Step G: (5-(difluoromethyl)-6-methyl-1-(tetrahydro-2H-pyran-2-yl)-1H-indazol-4-yl)boronic acid (Int-5)

To a solution of 4-bromo-5-(difluoromethyl)-6-methyl-1-(tetrahydro-2H-pyran-2-yl)-1H-indazole (Int-5F) (0.308 g, 0.892 mmol) in THF (8 mL) was added n-butyllithium (1.6 M in hexanes, 0.725 mL, 1.16 mmol) at −78° C. The resulting mixture was stirred at this temperature for 30 min. Then, triisopropyl borate (618 μL, 2.68 mmol) was added via syringe. The mixture was stirred at −78° C. for 10 min. The mixture was quenched with sat. aq. NH₄Cl (2 mL) and extracted with EtOAc (3×20 mL). The organic layer was dried over anhydrous Na₂SO₄, filtered, and concentrated in vacuo. (5-(difluoromethyl)-6-methyl-1-(tetrahydro-2H-pyran-2-yl)-1H-indazol-4-yl)boronic acid (Int-5) was obtained and used directly without further purification. MS (ESI): [M+H]⁺ m/z: 311.

### Intermediate 6: (5-(difluoromethoxv)-6-methyl-1-(tetrahydro-2H-pyran-2-yl)-1H-indazol-4-yl)boronic acid (Int-6)

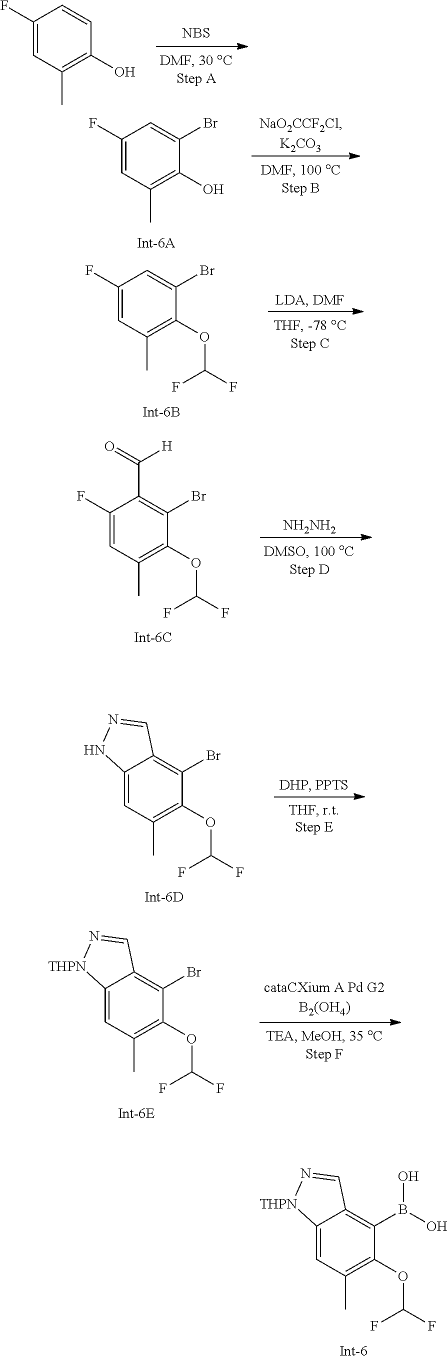

### Step A: 2-bromo-4-fluoro-6-methylphenol (Int-6A)

To a stirred solution of 4-fluoro-2-methylphenol (7.65 g, 60.7 mmol) in DMF (80 mL) was added NBS (10.8 g, 60.7 mmol), and the mixture was stirred at 30° C. for 18 h. The reaction mixture was quenched with water (10 mL) and extracted with EtOAc (3×80 mL). The combined organic layer was dried over Na₂SO₄, filtered, and the solvent was evaporated under reduced pressure. The residue was purified by silica gel chromatography (0 to 20% EtOAc in petroleum ether) to give 2-bromo-4-fluoro-6-methylphenol (Int-6A). ¹H NMR (400 MHz, CDCl₃) δ 7.04 (dd, J=2.7, 7.4 Hz, 1H), 6.83 (dd, J=2.9, 8.8 Hz, 1H), 2.28 (s, 3H).

### Step B: 1-bromo-2-(difluoromethoxv)-5-fluoro-3-methylbenzene (Int-6B)

To a mixture of 2-bromo-4-fluoro-6-methylphenol (Int-6A) (4.0 g, 19.5 mmol) in DMF (50 mL) was added sodium chlorodifluoroacetate (4.46 g, 29.3 mmol) and K₂CO₃ (8.09 g, 58.5 mmol), and the mixture was stirred at 100° C. for 15 h. The reaction mixture was quenched with water (30 mL), then the mixture was extracted with EtOAc (3×100 mL), the combined organic layers were washed with brine (50 mL), dried over Na₂SO₄, filtered, and concentrated in vacuo. The product was purified by silica gel chromatography (0 to 10% EtOAc in petroleum ether) to give 1-bromo-2-(difluoromethoxy)-5-fluoro-3-methylbenzene (Int-6B). ¹H NMR (400 MHz, CDCl₃) δ 7.18 (dd, J=3.1, 7.4 Hz, 1H), 6.92 (dd, J=2.7, 8.2 Hz, 1H), 6.48 (t, J=74.8, Hz, 1H), 2.36 (s, 3H).

### Step C: 2-bromo-3-(difluoromethoxy)-6-fluoro-4-methylbenzaldehyde (Int-6C)

To a solution of diisopropylamine (2.5 g, 25 mmol) in THF (50 mL) was added n-BuLi (7.8 mL, 19.5 mmol, 2.5 M in hexanes) slowly at 0° C., the mixture was stirred at 0° C. for 30 min, and then cooled to −78° C. to give LDA. Then a solution of 1-bromo-2-(difluoromethoxy)-5-fluoro-3-methylbenzene (Int-6B) (5.0 g, 20 mmol) in THF (5.0 mL) was added to the above mixture, and the resulting mixture was stirred at −78° C. for 40 min. DMF (5 mL) was added, and the mixture was stirred at −78° C. for another 10 min. The reaction mixture was quenched with water (2×5 mL) and extracted with EtOAc (3×60 mL). The combined organic layer was washed with brine (30 mL), dried over Na₂SO₄, filtered, and the solvent was evaporated under reduced pressure. The product was purified by silica gel chromatography (0 to 30% EtOAc in petroleum ether) to give 2-bromo-3-(difluoromethoxy)-6-fluoro-4-methylbenzaldehyde (Int-6C). ¹H NMR (400 MHz, CDCl₃) δ 10.30 (s, 1H), 7.07 (d, J=10.2 Hz, 1H), 6.53 (t, J=74.4 Hz, 1H), 2.45 (s, 3H).

### Step D: 4-bromo-5-(difluoromethoxy)-6-methyl-1H-indazole (Int-6D)

To a solution of 2-bromo-3-(difluoromethoxy)-6-fluoro-4-methylbenzaldehyde (Int-6C) (800 mg, 2.83 mmol) in DMSO (8.0 mL) was added hydrazine hydrate (832 mg, 14.1 mmol, 85% in H₂O), and the mixture was stirred at 100° C. for 15 h. The mixture was cooled and diluted with water (20 mL) and extracted with EtOAc (3×60 mL). The combined organic layer was dried over Na₂SO₄, filtered, and the solvent was evaporated under reduced pressure. The product was purified by silica gel chromatography (0 to 30% EtOAc in petroleum ether) to give 4-bromo-5-(difluoromethoxy)-6-methyl-1H-indazole (Int-6D). MS (ESI): m/z [M+H]⁺ 277.

### Step E: 4-bromo-5-(difluoromethoxv)-6-methyl-1-(tetrahydro-2H-pyran-2-yl)-1H-indazole (Int-6E)

To a solution of 4-bromo-5-(difluoromethoxy)-6-methyl-1H-indazole (Int-6D) (650 mg, 2.35 mmol) in THF (10.0 mL) was added pyridiniump-toluenesulfonate (59.0 mg, 0.235 mmol) and 3,4-dihydro-2H-pyran (987 mg, 11.7 mmol). The mixture was stirred at 25° C. for 15 h. The mixture was diluted with water (20 mL) and extracted with EtOAc (3×30 mL). The combined organic layer was dried over with Na₂SO₄, filtered, and the solvent was evaporated under reduced pressure. The product was purified by silica gel chromatography (0 to 30% EtOAc in petroleum ether) to give 4-bromo-5-(difluoromethoxy)-6-methyl-1-(tetrahydro-2H-pyran-2-yl)-1H-indazole (Int-6E). MS (ESI): m/z [M+H]⁺ 361.

### Step F: (5-(difluoromethoxv)-6-methyl-1-(tetrahydro-2H-pyran-2-yl)-1H-indazol-4-yl)boronic acid (Int-6)

To a solution of 4-bromo-5-(difluoromethoxy)-6-methyl-1-(tetrahydro-2H-pyran-2-yl)-1H-indazole (Int-6E) (2 g, 5.54 mmol) in MeOH (20 mL) was added tetrahydroxydiboron (1.99 g, 22.2 mmol), triethylamine (2.32 mL, 16.6 mmol) and CataCXium A Pd G2 (0.185 g, 0.277 mmol). The mixture was stirred at 35° C. for 2 h under N₂ atmosphere. The mixture was concentrated in vacuo and diluted with EtOAc (20 mL). The pH was adjusted to ~6 with 0.5 M aq. HCl in an ice-water bath and extracted with EtOAc (3×20 mL). The combined organic layers were dried over Na₂SO₄, filtered, and concentrated in vacuo. The residue was purified by flash silica gel chromatography (0 to 20% (EtOAc:EtOH=3:1) in petroleum ether) to give (5-(difluoromethoxy)-6-methyl-1-(tetrahydro-2H-pyran-2-yl)-1H-indazol-4-yl)boronic acid (Int-6). MS (ESI) [M+H]⁺: m/z 327.

### Intermediate 7: (5-(chlorodifluoromethoxy)-6-methyl-1-(tetrahydro-2H-pyran-2-yl)-1H-indazol-4-yl)boronic acid (Int-7)

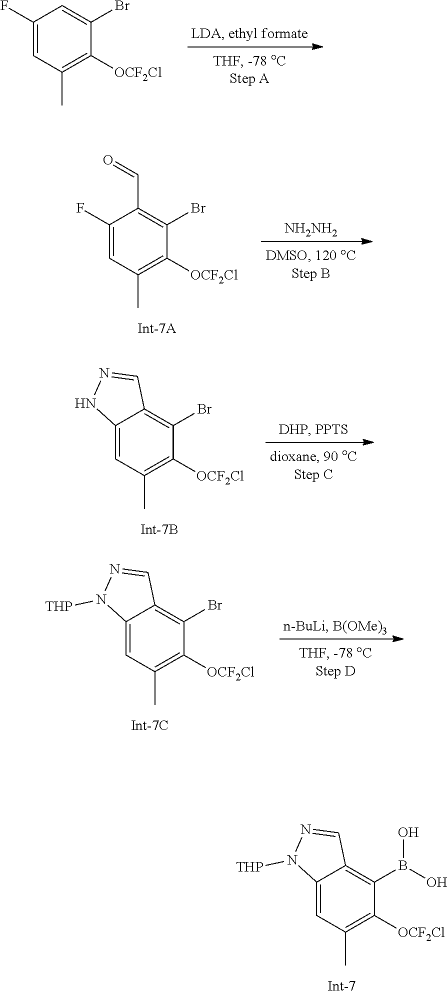

### Step A: 2-bromo-3-(chlorodifluoromethoxv)-6-fluoro-4-methylbenzaldehyde (Int-7A)

To a solution of 1-bromo-2-(chlorodifluoromethoxy)-5-fluoro-3-methylbenzene (1.00 g, 3.45 mmol) in THF (10 mL) was added lithium diisopropylamide (4.15 mL, 4.15 mmol, 1M in THF/hexanes) at −78° C. under N₂ atmosphere. The mixture was stirred at −78° C. for 1 h. To the reaction mixture was added ethyl formate (0.281 g, 3.80 mmol) at −78° C. and the resulting mixture was stirred at −78° C. for 0.5 h. The mixture was diluted with sat. aq. NH₄Cl (5 mL) and extracted with EtOAc (3×15 mL), dried over Na₂SO₄, filtered, and the solvent was evaporated under reduced pressure. The crude product was purified by flash silica gel chromatography (100% petroleum ether) to give 2-bromo-3-(chlorodifluoromethoxy)-6-fluoro-4-methylbenzaldehyde (Int-7A). ¹H NMR (400 MHz, CDCl₃) δ: 10.28-10.37 (m, 1H), 7.10 (d, J=10.4 Hz, 1H), 2.48 (s, 3H).

### Step B: 4-bromo-5-(chlorodifluoromethoxy)-6-methyl-1H-indazole (Int-7B)

To a solution of 2-bromo-3-(chlorodifluoromethoxy)-6-fluoro-4-methylbenzaldehyde (Int-7A) (0.9 g, 2.83 mmol) in DMSO (9 mL) was added hydrazine (0.534 g, 14.2 mmol, 85% in H₂O) at 20° C. under N₂ atmosphere. The mixture was stirred at 120° C. for 12 h. The mixture was cooled to room temperature, diluted with water (10 mL), and extracted with EtOAc (3×30 mL). The organic layer was washed with brine (3×10 mL), dried over Na₂SO₄, filtered, and the solvent was evaporated under reduced pressure. The crude product was purified by flash silica gel chromatography (0 to 10% EtOAc in petroleum ether) to give 4-bromo-5-(chlorodifluoromethoxy)-6-methyl-1H-indazole (Int-7B). MS (ESI) [M+H]⁺: m/z 311, 313.

### Step C: 4-bromo-5-(chlorodifluoromethoxy)-6-methyl-1-(tetrahydro-2H-pyran-2-yl)-1H-indazole (Int-7C)

To a solution of 4-bromo-5-(chlorodifluoromethoxy)-6-methyl-1H-indazole (Int-7B) (630 mg, 2.02 mmol) in dioxane (7 mL) was added pyridiniump-toluenesulfonate (102 mg, 0.404 mmol) and 3,4-dihydro-2H-pyran (340 mg, 4.04 mmol) at 20° C. The mixture was heated to 90° C. and stirred for 12 h. The mixture was cooled to room temperature and diluted with water (4 mL). The reaction mixture was extracted with EtOAc (3×10 mL), dried over Na₂SO₄, filtered, and concentrated in vacuo. The product was purified by flash silica gel chromatography (0 to 20% EtOAc in petroleum ether) to give 4-bromo-5-(chlorodifluoromethoxy)-6-methyl-1-(tetrahydro-2H-pyran-2-yl)-1H-indazole (Int-7C). MS (ESI) [M+H]⁺: m/z 395, 397.

### Step D: (5-(chlorodifluoromethoxy)-6-methyl-1-(tetrahydro-2H-pyran-2-yl)-1H-indazol-4-yl)boronic acid (Int-7)

To a solution of 4-bromo-5-(chlorodifluoromethoxy)-6-methyl-1-(tetrahydro-2H-pyran-2-yl)-1H-indazole (Int-7C) (550 mg, 1.39 mmol) in THF (10 mL) was added n-BuLi (0.834 mL, 2.09 mmol, 2.5 M in hexane) at −78° C. under N₂ atmosphere. The reaction was stirred at −78° C. for 10 min, then trimethyl borate (0.311 mL, 2.78 mmol) in THF (1 mL) was added dropwise. The reaction mixture was stirred at −78° C. for 1 h. The mixture was quenched with sat. aq. NH₄Cl (5 mL) at −78° C. The mixture was warmed to room temperature and extracted with EtOAc (2×20 mL). The combined organic layer was washed with brine (10 mL), dried over Na₂SO₄, and concentrated in vacuo. The residue was purified by flash silica gel chromatography (0 to 20% EtOAc in petroleum ether, then 10% (EtOAc/EtOH=3:1) in petroleum ether) to give (5-(chlorodifluoromethoxy)-6-methyl-1-(tetrahydro-2H-pyran-2-yl)-1H-indazol-4-yl)boronic acid (Int-7). MS (ESI) [M+H]⁺: m/z 361.

### Intermediate 8: (5-((triisopropylsilyl)ethynyl)-2-((2-(trimethylsilyl)ethoxy)methyl)-2H-thieno[3,2-]indazol-4-yl)boronic acid (Int-8)

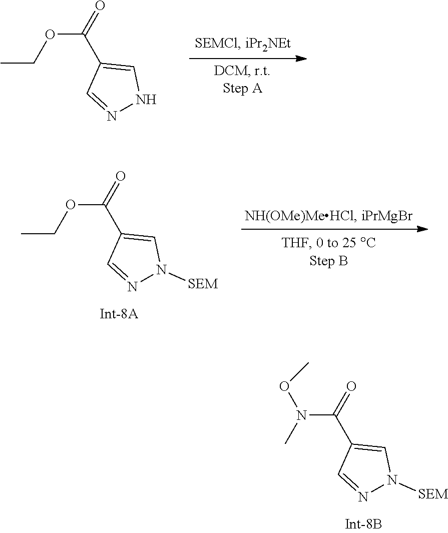

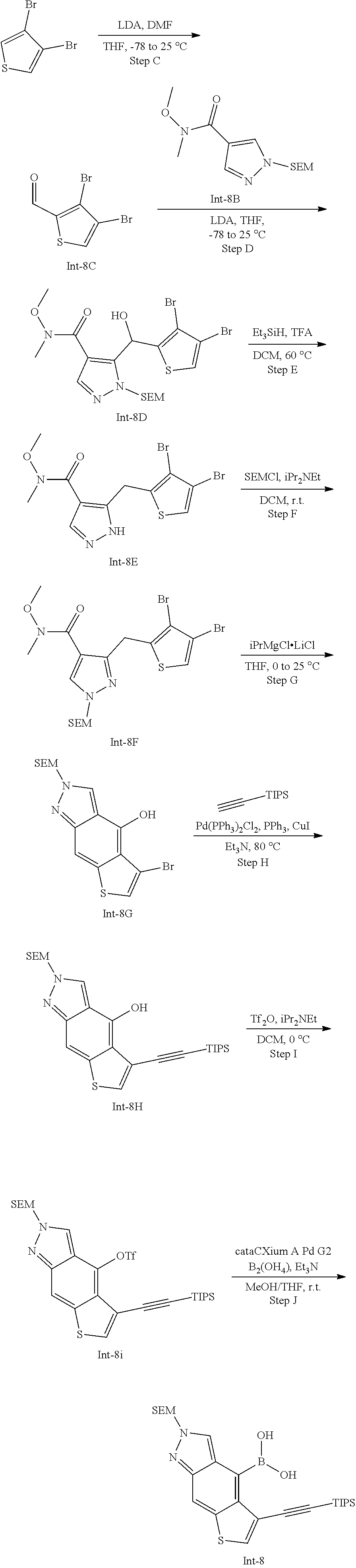

### Step A: Ethyl 1-((2-(trimethylsilyl)ethoxy)methyl)-1H-pyrazole-4-carboxylate (Int-8A)

To a solution of ethyl 1H-pyrazole-4-carboxylate (13.8 g, 98 mmol) in DCM (100 mL) at 0° C. was added N,N-diisopropylethylamine (25.4 g, 196 mmol). The mixture was stirred for 15 min, then (2-(chloromethoxy)ethyl)trimethylsilane (24.5 g, 147 mmol) was added slowly. The mixture was warmed to room temperature and stirred for 16 h. The mixture was quenched with water (50 mL) and extracted with DCM (3×200 mL). The combined organic layer was dried over Na₂SO₄, filtered, and the filtrate was evaporated under reduced pressure. The crude product was purified by flash silica gel chromatography (0 to 35% EtOAc in petroleum ether) to give ethyl 1-((2-(trimethylsilyl)ethoxy)methyl)-1H-pyrazole-4-carboxylate (Int-8A). MS (ESI) [M+H]⁺: m/z 271.

### Step B: N-methoxy-N-methyl-1-((2-(trimethylsilyl)ethoxy)methyl)-1H-pyrazole-4-carboxamide (Int-8B)

To a mixture of ethyl 1-((2-(trimethylsilyl)ethoxy)methyl)-1H-pyrazole-4-carboxylate (Int-8A) (20 g, 74.0 mmol) and N, O-dimethylhydroxylamine hydrochloride (10.8 g, 111 mmol) in dry THF (350 mL) was added iPrMgBr (77 mL, 222 mmol, 2.9 M in Me-THF) at 0° C. The mixture was warmed to 25° C. and stirred for 16 h. The mixture was quenched with sat. aq. NH₄Cl (100 mL) and extracted with EtOAc (3×300 mL). The organic layer was dried over Na₂SO₄, filtered, and the filtrate was evaporated under reduced pressure. The product was purified by flash silica gel chromatography (0 to 50% EtOAc in petroleum ether) to give N-methoxy-N-methyl-1-((2-(trimethylsilyl)ethoxy)methyl)-1H-pyrazole-4-carboxamide (Int-8B). MS (ESI) [M+H]⁺: m/z 286.

### Step C: 3,4-dibromothiophene-2-carbaldehyde (Int-8C)

To the solution of 3,4-dibromothiophene (12.4 g, 51.2 mmol) in THF (120 mL) was added LDA (102 mL, 102 mmol, 1M in THF) at −78° C., and the reaction mixture was stirred at −78° C. for 2 h. N,N-dimethylformamide (4.73 mL, 61.4 mmol) was added dropwise to the reaction mixture, warmed to 25° C., and stirred for 1 h. The mixture was quenched with aqueous NH₄Cl (100 mL) and extracted with EtOAc (3×200 mL). The combined organic layer was washed with brine (2×50 mL), dried over Na₂SO₄, filtered, and evaporated under reduced pressure. The product was purified by flash silica gel chromatography (0 to 50% EtOAc in petroleum ether) to give 3,4-dibromothiophene-2-carbaldehyde (Int-8C). ¹H NMR (400 MHz, CDCl₃) δ: 9.96 (d, J=1.4 Hz, 1H), 7.76 (d, J=1.3 Hz, 1H).

### Step D: 5-((3,4-dibromothiophen-2-yl)(hydroxy)methyl)-N-methoxy-N-methyl-1-((2-(trimethylsilyl)ethoxy)methyl)-1H-pyrazole-4-carboxamide (Int-8D)

To a stirred solution of N-methoxy-N-methyl-1-((2-(trimethylsilyl)ethoxy)methyl)-1H-pyrazole-4-carboxamide (Int-8B) (6.20 g, 21.7 mmol) in THF (150 mL) was added lithium diisopropylamide (22.81 mL, 22.81 mmol, 1M in THF) at −78° C. under N₂ atmosphere and the reaction was stirred at −78° C. for 0.5 h. 3,4-dibromothiophene-2-carbaldehyde (Int-8C) (8.80 g, 32.6 mmol) in THF (10 mL) was added at −78° C. and the reaction was warmed 25° C. and stirred for 16 h. The mixture was quenched with sat. aq. NH₄Cl (40 mL) and extracted with EtOAc (3×200 mL). The organic layers were washed with brine (2×50 mL), dried over Na₂SO₄, filtered, and the solvent was evaporated under reduced. The residue was purified by flash silica gel chromatography (0 to 50% EtOAc in petroleum ether) to give 5-((3,4-dibromothiophen-2-yl)(hydroxy)methyl)-N-methoxy-N-methyl-1-((2-(trimethylsilyl)ethoxy)methyl)-1H-pyrazole-4-carboxamide (Int-8D). ¹H NMR (400 MHz, CDCl₃) δ: 8.04 (s, 1H), 7.28 (s, 1H), 6.39 (s, 1H), 5.56 (d, J=8.1 Hz, 2H), 3.70 (s, 3H), 3.39-3.55 (m, 2H), 3.37 (s, 3H), 0.60-0.92 (m, 2H), −0.05 (s, 9H).

### Step E: 5-((3,4-dibromothiophen-2-yl)methyl)-N-methoxy-N-methyl-1H-pyrazole-4-carboxamide (Int-8E)

To a solution of 5-((3,4-dibromothiophen-2-yl)(hydroxy)methyl)-N-methoxy-N-methyl-1-((2-(trimethylsilyl)ethoxy)methyl)-1H-pyrazole-4-carboxamide (Int-8D) (7.38 g, 13.3 mmol) in DCM (18 mL) was added triethylsilane (36.5 mL, 229 mmol) and TFA (18.3 mL, 237 mmol) at 25° C. The mixture was warmed to 60° C. and stirred for 5 h. After 5 h, the reaction mixture was cooled to room temperature and concentrated in vacuo. The residue was diluted with DCM (50 mL) and adjusted to pH ~8 with sat. aq. NaHCO₃. The layers were separated and the organic layer was washed with brine (2×30 mL), dried with Na₂SO₄, filtered, and concentrated in vacuo. The residue was purified by flash silica gel chromatography (0 to 100% EtOAc in petroleum ether) to give 5-((3,4-dibromothiophen-2-yl)methyl)-N-methoxy-N-methyl-1H-pyrazole-4-carboxamide (Int-8E). ¹H NMR (400 MHz, CDCl₃) δ: 8.07 (s, 1H), 7.25 (s, 1H), 4.63 (s, 2H), 3.69 (s, 3H), 3.36 (s, 3H).

### Step F: 3-((3,4-dibromothiophen-2-yl)methyl)-N-methoxy-N-methyl-1-((2-(trimethylsilyl)ethoxy)methyl)-1H-pyrazole-4-carboxamide (Int-8F)

To a solution of 5-((3,4-dibromothiophen-2-yl)methyl)-N-methoxy-N-methyl-1H-pyrazole-4-carboxamide (Int-8E) (4.85 g, 11.9 mmol) in DCM (50 mL) was added N,N-diisopropylethylamine (3.06 g, 23.7 mmol), and the mixture was stirred at 0° C. for 15 min. (2-(chloromethoxy)ethyl)trimethylsilane (2.96 g, 17.8 mmol) was added slowly at 0° C. The mixture was warmed to 25° C. and stirred for 2 h. The mixture was quenched with H₂O (10 mL) and extracted with DCM (3×50 mL). The combined organic layer was dried over Na₂SO₄, filtered, and the filtrate was evaporated under reduced pressure. The product was purified by flash silica gel chromatography (0 to 100% EtOAc in petroleum ether) to give 3-((3,4-dibromothiophen-2-yl)methyl)-N-methoxy-N-methyl-1-((2-(trimethylsilyl)ethoxy)methyl)-1H-pyrazole-4-carboxamide (Int-8F). MS (ESI) [M+H]⁺: m/z 538, 540, 542.

### Step G: 5-bromo-2-((2-(trimethylsilyl)ethoxy)methyl)-2H-thieno[3,2-f]indazol-4-ol (Int-8G)

To a stirred solution of 3-((3,4-dibromothiophen-2-yl)methyl)-N-methoxy-N-methyl-1-((2-(trimethylsilyl)ethoxy)methyl)-1H-pyrazole-4-carboxamide (Int-8F) (2.00 g, 3.71 mmol) in THF (20 mL) was added dropwise isopropylmagnesium chloride lithium chloride complex (5.70 mL, 7.42 mmol, 1.3M in THF) at 0° C. The reaction was warmed to 25° C. and stirred for 16 h under N₂ atmosphere. The mixture was quenched with sat. aq. NH₄Cl (15 mL) and extracted with EtOAc (3×50 mL). The organic layer was dried over Na₂SO₄, filtered, and the filtrate was evaporated under reduced pressure. The crude product was purified by flash silica gel chromatography (0 to 35% EtOAc in petroleum ether) to give 5-bromo-2-((2-(trimethylsilyl)ethoxy)methyl)-2H-thieno[3,2-f]indazol-4-ol (Int-8G). MS (ESI) [M+H]⁺: m/z 399, 401.

### Step H: 5-((triisopropylsilyl)ethynyl)-2-((2-(trimethylsilyl)ethoxy)methyl)-2H-thieno[3,2-f]indazol-4-ol (Int-8H)

To a solution of 5-bromo-2-((2-(trimethylsilyl)ethoxy)methyl)-2H-thieno[3,2-f]indazol-4-ol (Int-8G) (620 mg, 1.55 mmol) in triethylamine (15 mL) was added ethynyltriisopropylsilane (425 mg, 2.33 mmol), copper(I) iodide (29.6 mg, 0.155 mmol), triphenylphosphine (40.7 mg, 0.155 mmol), and bis(triphenylphosphine)palladium(II) dichloride (54.5 mg, 0.078 mmol) at 25° C. under N₂ atmosphere. The mixture was heated to 80° C. and stirred for 2 h. The reaction mixture was cooled and evaporated under reduced pressure. The crude product was purified by flash silica gel chromatography (0 to 10% EtOAc in petroleum ether) to give 5-((triisopropylsilyl)ethynyl)-2-((2-(trimethylsilyl)ethoxy)methyl)-2H-thieno[3,2-]indazol-4-ol (Int-8H). MS (ESI) [M+H]⁺: m/z 501.

### Step I: 5-((triisopropylsilyl)ethynyl)-2-((2-(trimethylsilyl)ethoxy)methyl)-2H-thieno[3,2-f]indazol-4-yl trifluoromethanesulfonate (Int-8I)

To a solution of 5-((triisopropylsilyl)ethynyl)-2-((2-(trimethylsilyl)ethoxy)methyl)-2H-thieno[3,2-]indazol-4-ol (Int-8H) (442 mg, 0.883 mmol) in DCM (8 mL) under N₂ atmosphere was added N,N-diisopropylethylamine (0.462 mL, 2.65 mmol) at 0° C. and the mixture was stirred at 0° C. for 10 min. Trifluoromethanesulfonic anhydride (0.224 mL, 1.32 mmol) was added to the above mixture. The reaction was stirred at 0° C. for 1 h under N₂ atmosphere. The mixture was warmed to room temperature and concentrated in vacuo. The product was purified by flash silica gel chromatography (petroleum ether/EtOAc=10/1) to give 5-((triisopropylsilyl)ethynyl)-2-((2-(trimethylsilyl)ethoxy)methyl)-2H-thieno[3,2-f]indazol-4-yl trifluoromethanesulfonate (Int-8I). MS (ESI) [M+H]⁺: m/z 633.

### Step J: (5-((triisopropylsilyl)ethynyl)-2-((2-(trimethylsilyl)ethoxy)methyl)-2H-thieno[3,2-f]indazol-4-yl)boronic acid (Int-8)

To a solution of 5-((triisopropylsilyl)ethynyl)-2-((2-(trimethylsilyl)ethoxy)methyl)-2H-thieno[3,2-J]indazol-4-yl trifluoromethanesulfonate (Int-8I) (424 mg, 0.670 mmol) in MeOH/THF (5 mL, 3:1 v/v) was added TEA (360 mg, 4.02 mmol), CataCXium A Pd G2 (44.8 mg, 0.067 mmol), and tetrahydroxydiboron (339 mg, 3.35 mmol), and the mixture was stirred at 25° C. for 2 h. The mixture was diluted with water (3 mL) and extracted with EtOAc (3×10 mL). The organic layer was dried over Na₂SO₄, filtered, and the filtrate was evaporated under reduced pressure. The product was purified by flash silica gel chromatography (0 to 35% EtOAc in petroleum ether) to give (5-((triisopropylsilyl)ethynyl)-2-((2-(trimethylsilyl)ethoxy)methyl)-2H-thieno[3,2-J]indazol-4-yl)boronic acid (Int-8). MS (ESI) [M+H]⁺: m/z 529.

### Intermediate 23: 4-(5,5-dimethyl-1,3,2-dioxaborinan-2-yl)-6-fluoro-5-((triisopropylsilyl)ethynyl)-1-((2-(trimethylsilyl)ethoxy)methyl)-1H-thieno[3,2-f]indazole (Int-23)

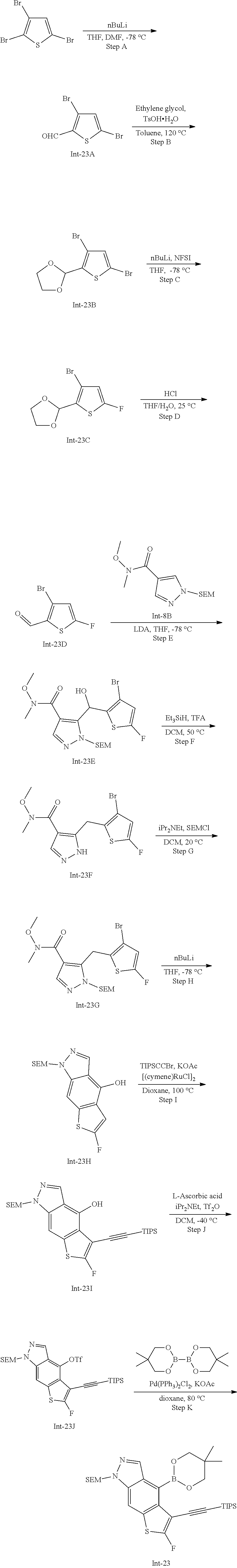

### Step A: 3,5-dibromothiophene-2-carbaldehyde (Int-23A)

To a solution of 2,3,5-tribromothiophene (100 g, 312 mmol) in THF (1.1 L) was added nBuLi (125 mL, 312 mmol, 2.5 M in hexane) dropwise at −78° C. under N₂. The mixture was stirred at −78° C. for 1 h, then DMF (29.0 mL, 374 mmol) was added to the reaction mixture. The mixture was stirred at −78° C. for 1.5 h. The mixture was quenched with ice water (500 mL), then treated with DCM (500 mL). The organic layer was separated, and the aqueous layer was extracted with DCM (3×500 mL). The combined organic layers were dried over anhydrous sodium sulfate, filtered, and concentrated in vacuo to give a residue. The residue was diluted with petroleum ether (50 mL), stirred at 25° C. for 30 min, then filtered. The filter cake was washed with petroleum ether (3×30 mL) to give 3,5-dibromothiophene-2-carbaldehyde (Int-23A). MS (ESI) [M+H]⁺: m/z 269, 271.

### Step B: 2-(3,5-dibromothiophen-2-yl)-1,3-dioxolane (Int-23B)

To a solution of 3,5-dibromothiophene-2-carbaldehyde (48 g, 178 mmol) (Int-23A) in toluene (480 mL) was added ethylene glycol (39.7 mL, 711 mmol) and TsOH·H₂O (1.691 g, 8.89 mmol) at 25° C. The reaction was stirred at 120° C. for 16 h under N₂. The reaction mixture was cooled to room temperature and quenched with sat. aq. NaHCO₃ (200 mL). The reaction was treated with H₂O (100 mL) and DCM (300 mL). The organic layer was separated, and the aqueous layer was extracted with DCM (3×300 mL). The combined organic layers were washed with brine (300 mL), dried over anhydrous sodium sulfate, filtered, and concentrated in vacuo to give a crude residue. The residue was purified by flash silica gel chromatography (3% EtOAc in petroleum ether) to give 2-(3,5-dibromothiophen-2-yl)-1,3-dioxolane (Int-23B). MS (ESI) [M+H]⁺: m/z 313, 315.

### Step C: 2-(3-bromo-5-fluorothiophen-2-yl)-1,3-dioxolane (Int-23C)

To a solution of 2-(3,5-dibromothiophen-2-yl)-1,3-dioxolane (Int-23B) (40.66 g, 129 mmol) in THF (400 mL) was added nBuLi (51.8 mL, 129 mmol, 2.5 M in hexane) dropwise at −78° C. under N₂. The mixture was stirred at −78° C. for 30 min. A solution of NFSI (82 g, 259 mmol) in THF (200 mL) was added to the reaction mixture. The mixture was stirred at −78° C. for 10 min under N₂. The mixture was quenched with sat. aq. NH₄Cl (400 mL). The mixture was treated with H₂O (100 mL) and EtOAc (400 mL). The organic layer was separated, and the aqueous layer was extracted with EtOAc (3×400 mL). The combined organic layers were washed with brine (400 mL), dried over sodium sulfate, filtered, and concentrated under reduced pressure. The residue was purified by flash silica gel chromatography (0 to 5% EtOAc in petroleum ether) to give 2-(3-bromo-5-fluorothiophen-2-yl)-1,3-dioxolane (Int-23C). MS (ESI) [M+H]⁺: m/z 253, 255.

### Step D: 3-bromo-5-fluorothiophene-2-carbaldehyde (Int-23D)

A solution of 2-(3-bromo-5-fluorothiophen-2-yl)-1,3-dioxolane (Int-23C) (14.0 g, 55.3 mmol) in THF (300 mL) and 1 M HCl (100 mL) was stirred at 25° C. for 16 h under N₂. The reaction was treated with H₂O (100 mL) and EtOAc (100 mL). The organic layer was separated, and the aqueous layer was extracted with EtOAc (3×100 mL). The combined organic layers were washed with brine (100 mL), dried over sodium sulfate, filtered, and concentrated in vacuo. The material was purified by flash silica gel chromatography (0 to 5% DCM in petroleum ether). The material was repurified by reverse-phase MPLC (0 to 35% MeCN in H₂O w/0.5% TFA modifier) to give 3-bromo-5-fluorothiophene-2-carbaldehyde (Int-23D). MS (ESI) [M+H]⁺: m/z 209, 211.

### Step E: 5-((3-bromo-5-fluorothiophen-2-yl)(hydroxy)methyl)-N-methoxy-N-methyl-1-((2-(trimethylsilyl)ethoxy)methyl)-1H-pyrazole-4-carboxamide (Int-23E)

To a stirred solution of N-methoxy-N-methyl-1-((2-(trimethylsilyl)ethoxy)methyl)-1H-pyrazole-4-carboxamide (Int-8B) (13.7 g, 48.0 mmol) in THF (130 mL) was added LDA (27.6 mL, 55.2 mmol, 1 M in THF) dropwise at −78° C. under N₂. The reaction was stirred at −78° C. for 0.5 h. 3-bromo-5-fluorothiophene-2-carbaldehyde (Int-23D) (5.02 g, 24.0 mmol) in THF (25 mL) was added at −78° C. The reaction was stirred at −78° C. for 10 min. The mixture was quenched with sat. aq. NH₄Cl (50 mL). The reaction was treated with H₂O (50 mL) and EtOAc (100 mL). The organic layer was separated, and the aqueous layer was extracted with EtOAc (3×50 mL). The combined organic layers were washed with brine (50 mL), dried over anhydrous sodium sulfate, filtered, and concentrated in vacuo. The material was purified by flash silica gel chromatography (0 to 20% EtOAc in petroleum ether) to give 5-((3-bromo-5-fluorothiophen-2-yl)(hydroxy)methyl)-N-methoxy-N-methyl-1-((2-(trimethylsilyl)ethoxy)methyl)-1H-pyrazole-4-carboxamide (Int-23E). ¹H NMR (400 MHz, CDCl₃) δ 8.08 (s, 1H), 7.51 (d, J=11.2 Hz, 1H), 6.36-6.42 (m, 2H), 5.54-5.70 (m, 2H), 3.77 (s, 3H), 3.47-3.60 (m, 2H), 3.44 (s, 3H), 0.79-0.93 (m, 2H), 0.00 (s, 9H).

### Step F: 5-((3-bromo-5-fluorothiophen-2-yl)methyl)-N-methoxy-N-methyl-1H-pyrazole-4-carboxamide (Int-23F)

To a solution of 5-((3-bromo-5-fluorothiophen-2-yl)(hydroxy)methyl)-N-methoxy-N-methyl-1-((2-(trimethylsilyl)ethoxy)methyl)-1H-pyrazole-4-carboxamide (Int-23E) (7.9 g, 16 mmol) in DCM (20 mL) was added Et₃SiH (40 mL) and TFA (20 mL) at 25° C. The reaction was stirred at 50° C. for 2 h under N₂. The mixture was cooled to room temperature and evaporated under reduced pressure. The residue was diluted with EtOAc (80 mL) and adjusted to pH~7 with sat. aq. NaHCO₃ (80 mL). The organic layer was separated, and the aqueous layer was extracted with EtOAc (3×50 mL). The combined organic layers were washed with brine (50 mL), dried over sodium sulfate, filtered, and concentrated in vacuo. The product was suspended in petroleum ether (50 mL), filtered, and washed with petroleum ether (20 mL) to give 5-((3-bromo-5-fluorothiophen-2-yl)methyl)-N-methoxy-N-methyl-1H-pyrazole-4-carboxamide (Int-23F). MS (ESI) [M+H]⁺: m/z 348, 350.

### Step G: 5-((3-bromo-5-fluorothiophen-2-yl)methyl)-N-methoxy-N-methyl-1-((2-(trimethylsilyl)ethoxy)methyl)-1H-pyrazole-4-carboxamide (Int-23G)

To a stirred solution of 5-((3-bromo-5-fluorothiophen-2-yl)methyl)-N-methoxy-N-methyl-1H-pyrazole-4-carboxamide (Int-23F) (4.63 g, 13.3 mmol) in DCM (60 mL) was added N,N-diisopropylethylamine (4.63 mL, 26.6 mmol) and SEMCl (3.69 mL, 20 mmol) at 20° C. under N₂. The reaction was stirred at 20° C. for 12 h. The reaction was treated with H₂O (50 mL) and DCM (60 mL). After filtration, the organic layer was separated, and the aqueous layer was extracted with EtOAc (3×60 mL). The combined organic layers were washed with sat. aq. NaHCO₃ (50 mL), dried over anhydrous sodium sulfate, filtered, and concentrated in vacuo. The residue was purified by flash silica gel chromatography (0 to 20% EtOAc in petroleum ether) to give 5-((3-bromo-5-fluorothiophen-2-yl)methyl)-N-methoxy-N-methyl-1-((2-(trimethylsilyl)ethoxy)methyl)-1H-pyrazole-4-carboxamide (Int-23G). MS (ESI) [M+H]⁺: m/z 478, 480.

### Step H: 6-fluoro-1-((2-(trimethylsilyl)ethoxy)methyl)-1H-thieno[3,2-f]indazol-4-ol (Int-23H)

To a solution of 5-((3-bromo-5-fluorothiophen-2-yl)methyl)-N-methoxy-N-methyl-1-((2-(trimethylsilyl)ethoxy)methyl)-1H-pyrazole-4-carboxamide (Int-23G) (1.60 g, 3.34 mmol) in THF (20 mL) was added nBuLi (2.00 mL, 5.02 mmol) at −78° C. dropwise under N₂. The reaction was stirred at −78° C. for 0.5 h. The mixture was quenched with sat. aq. NH₄Cl (20 mL). The mixture was treated with H₂O (20 mL) and EtOAc (20 mL). The organic layer was separated, and the aqueous layer was extracted with EtOAc (3×20 mL). The combined organic layers were washed with brine (20 mL), dried over anhydrous sodium sulfate, filtered, and concentrated. The residue was purified by flash silica gel chromatography (0 to 20% THF in petroleum ether) to provide 6-fluoro-1-((2-(trimethylsilyl)ethoxy)methyl)-1H-thieno[3,2-f]indazol-4-ol (Int-23H). MS (ESI) [M+H]⁺: m/z 339.

### Step I: 6-fluoro-5-((triisopropylsilyl)ethynyl)-1-((2-(trimethylsilyl)ethoxy)methyl)-1H-thieno[3,2-f]indazol-4-ol (Int-23I)

To a solution of 6-fluoro-1-((2-(trimethylsilyl)ethoxy)methyl)-1H-thieno[3,2-f]indazol-4-ol (Int-23H) (900 mg, 2.66 mmol) and [(cymene)RuCl]₂ (814 mg, 1.33 mmol) in dioxane (36 mL) was added (bromoethynyl)triisopropylsilane (2.50 g, 9.57 mmol) and KOAc (992 mg, 10.1 mmol) at 25° C. under N₂. The mixture was stirred at 100° C. for 2 h. The mixture was cooled and diluted with EtOAc (20 mL). The resulting mixture was filtered and concentrated in vacuo. The residue was purified by flash silica gel chromatography (0 to 1% EtOAc in DCM) to give 6-fluoro-5-((triisopropylsilyl)ethynyl)-1-((2-(trimethylsilyl)ethoxy)methyl)-1H-thieno[3,2-f]indazol-4-ol (Int-23I). MS (ESI) [M+H]⁺: m/z 519.

### Step J: 6-fluoro-5-((triisopropylsilyl)ethynyl)-1-((2-(trimethylsilyl)ethoxy)methyl)-1H-thieno[3,2-f]indazol-4-yl trifluoromethanesulfonate (Int-23J)

To a solution of 6-fluoro-5-((triisopropylsilyl)ethynyl)-1-((2-(trimethylsilyl)ethoxy)methyl)-1H-thieno[3,2-]indazol-4-ol (Int-23I) (1.00 g, 1.927 mmol) and L-ascorbic acid (0.017 g, 0.096 mmol) in DCM (20 mL) was added N,N-diisopropylethylamine (2.02 mL, 11.6 mmol) at −40° C. Tf₂O (0.98 mL, 5.78 mmol) was added to the above mixture at −40° C. The reaction was stirred at −40° C. for 0.5 h under N₂. The mixture was concentrated in vacuo to give a residue. The residue was purified by flash silica gel chromatography (0 to 10% EtOAc in petroleum ether) to give 6-fluoro-5-((triisopropylsilyl)ethynyl)-1-((2-(trimethylsilyl)ethoxy)methyl)-1H-thieno[3,2-J]indazol-4-yl trifluoromethanesulfonate (Int-23J). MS (ESI) [M+H]⁺: m/z 651.

### Step K: 4-(5,5-dimethyl-1,3,2-dioxaborinan-2-yl)-6-fluoro-5-((triisopropylsilyl)ethynyl)-1-((2-(trimethylsilyl)ethoxy)methyl)-1H-thieno[3,2-f]indazole (Int-23)

To a solution of 6-fluoro-5-((triisopropylsilyl)ethynyl)-1-((2-(trimethylsilyl)ethoxy)methyl)-1H-thieno[3,2-]indazol-4-yl trifluoromethanesulfonate (Int-23J) (900 mg, 1.38 mmol) in dioxane (30 mL) was added KOAc (407 mg, 4.15 mmol) and bis(neopentyl glycolato)diboron (937 mg, 4.15 mmol) at 25° C. under N₂. The reaction was stirred at 25° C. for 10 min. Pd(PPh₃)₂Cl₂ (97 mg, 0.14 mmol) was added to the reaction under N₂. The reaction was stirred at 80° C. for 3 h. The mixture was cooled and diluted with EtOAc (20 mL). The resulting mixture was filtered and concentrated in vacuo. The residue was purified by flash silica gel chromatography (0 to 10% EtOAc in petroleum ether) to give 4-(5,5-dimethyl-1,3,2-dioxaborinan-2-yl)-6-fluoro-5-((triisopropylsilyl)ethynyl)-1-((2-(trimethylsilyl)ethoxy)methyl)-1H-thieno[3,2-f]indazole (Int-23). MS (ESI) [M+H]⁺: m/z 615.

The compounds in the table below were synthesized using a similar procedure as described in the synthesis of Int-23 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | Starting | | | [M + H]⁺ | |
|---|---|---|---|---|---|
| Int. | Material | Structure | Compound Name | Found | Conditions |
| Int-47 | 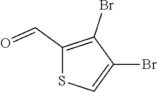 3,4- dibromothio- | 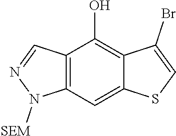 [SMILES: C[Si](C)(C)CCOCn1ncc2c(O)c3c(Br)csc3cc21] | 5-bromo-1-((2- (trimethylsilyl)ethoxy) methyl)-1H-thieno[3,2- f]indazol-4-ol | 399 | Made following steps E to H |
| | phene-2- | | | | |
| | carbaldehyde | | | | |

### Intermediate 48: (5-((triisopropylsilyl)ethynyl)-1-((2-(trimethylsilyl)ethoxy)methyl)-1H-thieno[3,2-f]indazol-4-yl)boronic acid (Int-48)

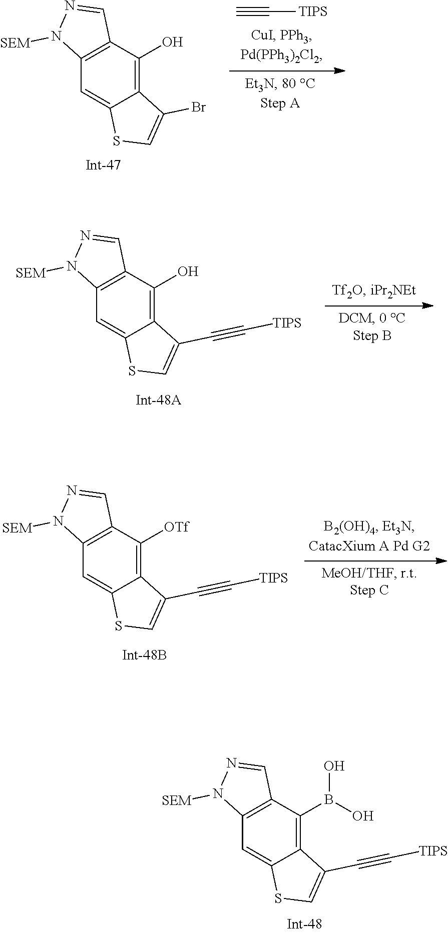

### Step A: 5-((triisopropylsilyl)ethynyl)-1-((2-(trimethylsilyl)ethoxy)methyl)-1H-thieno[3,2-f]indazol-4-ol (Int-48A)

To a solution of 5-bromo-1-((2-(trimethylsilyl)ethoxy)methyl)-1H-thieno[3,2-f]indazol-4-ol (Int-47) (620 mg, 1.552 mmol) in triethylamine (15 mL) was added ethynyltriisopropylsilane (425 mg, 2.329 mmol), copper(I) iodide (29.6 mg, 0.155 mmol), triphenylphosphine (40.7 mg, 0.155 mmol), and bis(triphenylphosphine)palladium(II) dichloride (54.5 mg, 0.078 mmol) at 25° C. under N₂. The mixture was stirred at 80° C. for 2 h. The mixture was cooled and concentrated under reduced pressure. The product was purified by flash silica gel chromatography (0 to 10% EtOAc in petroleum ether) to give 5-((triisopropylsilyl)ethynyl)-1-((2-(trimethylsilyl)ethoxy)methyl)-1H-thieno[3,2-J]indazol-4-ol (Int-48A). MS (ESI) [M+H]⁺: m/z 501.

### Step B: 5-((triisopropylsilyl)ethynyl)-1-((2-(trimethylsilyl)ethoxy)methyl)-1H-thieno[3,2-f]indazol-4-yl trifluoromethanesulfonate (Int-48B)

To a solution of 5-((triisopropylsilyl)ethynyl)-1-((2-(trimethylsilyl)ethoxy)methyl)-1H-thieno[3,2-]indazol-4-ol (Int-48A) (442 mg, 0.883 mmol) in DCM (8 mL) was added N,N-diisopropylethylamine (0.462 mL, 2.65 mmol) at 0° C. and the mixture was stirred at 0° C. for 10 min. Tf₂O (0.224 mL, 1.324 mmol) was added and the reaction was stirred at 0° C. for 1 h. The reaction mixture was concentrated in vacuo. The product was purified by flash silica gel chromatography (petroleum ether/EtOAc=10/1) to give 5-((triisopropylsilyl)ethynyl)-1-((2-(trimethylsilyl)ethoxy)methyl)-1H-thieno[3,2-]indazol-4-yl trifluoromethanesulfonate (Int-48B). MS (ESI) [M+H]⁺: m/z 633.

### Step C: (5-((triisopropylsilyl)ethynyl)-1-((2-(trimethylsilyl)ethoxy)methyl)-1H-thieno[3,2-f]indazol-4-yl)boronic acid (Int-48)

To a solution of 5-((triisopropylsilyl)ethynyl)-1-((2-(trimethylsilyl)ethoxy)methyl)-1H-thieno[3,2-]indazol-4-yl trifluoromethanesulfonate (Int-48B) (424 mg, 0.670 mmol) in MeOH/THF (5 mL, 3/1) was added hypodiboric acid (360 mg, 4.02 mmol), cataCXium A Pd G2 (44.8 mg, 0.067 mmol), and triethylamine (339 mg, 3.35 mmol). The mixture was stirred at 25° C. for 2 h under N₂. The mixture was diluted with water (3 mL) and extracted with EtOAc (3×). The organic layer was dried over sodium sulfate, filtered, and the filtrate was evaporated under reduced pressure. The product was purified by flash silica gel chromatography (0 to 35% EtOAc/petroleum ether) to give (5-((triisopropylsilyl)ethynyl)-1-((2-(trimethylsilyl)ethoxy)methyl)-1H-thieno[3,2-f]indazol-4-yl)boronic acid (Int-48). MS (ESI) [M+H]⁺: m/z 529.

### Intermediate 9: 5-chloro-4-(4,4,5,5-tetramethyl-1,3,2-dioxaborolan-2-yl)-1-((trifluoromethyl)sulfonyl)-1H-benzo[f]indazole (Int-9)

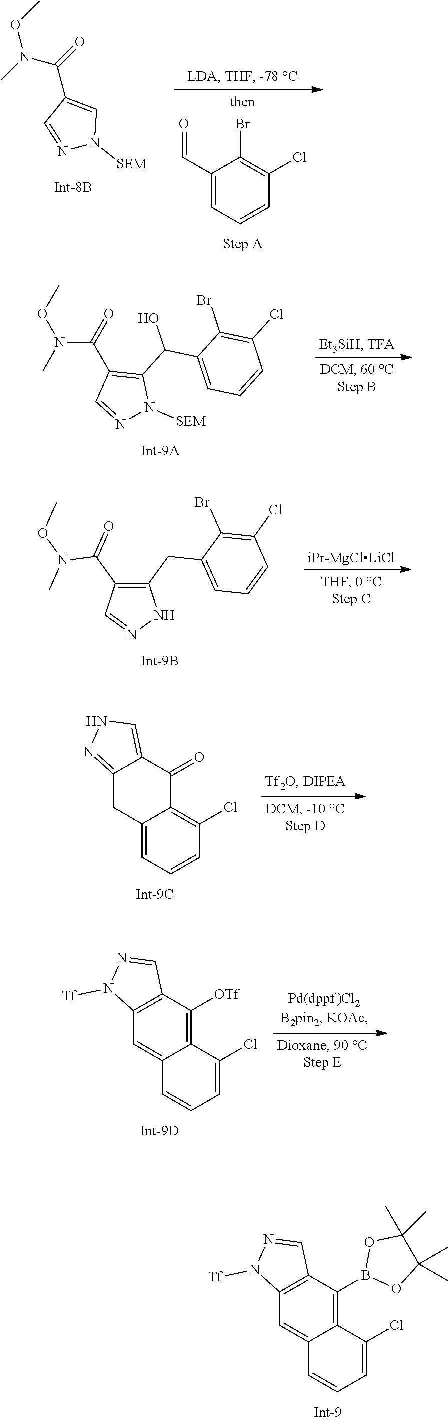

### Step A: 5-((2-bromo-3-chlorophenyl)(hydroxy)methyl)-N-methoxy-N-methyl-1-((2-(trimethylsilyl)ethoxy)methyl)-1H-pyrazole-4-carboxamide (Int-9A)

To a stirred solution of N-methoxy-N-methyl-1-((2-(trimethylsilyl)ethoxy)methyl)-1H-pyrazole-4-carboxamide (Int-8B) (5 g, 17.52 mmol) in THF (50 mL) was added lithium diisopropylamide (9.20 mL, 18.39 mmol, 2 M in THF) at −78° C. under N₂. The reaction was stirred at −78° C. for 0.5 h. 2-bromo-3-chlorobenzaldehyde (5.77 g, 26.3 mmol) was added at −78° C. and the reaction was stirred at −78° C. for 0.5 h under N₂ atmosphere. The mixture was quenched with sat. aq. NH₄Cl (20 mL) and extracted with EtOAc (3×40 mL). The combined organic layer was washed with brine (20 mL), dried over Na₂SO₄, and concentrated. The residue was purified by flash silica gel chromatography (0 to 36% EtOAc in petroleum ether) to give 5-((2-bromo-3-chlorophenyl)(hydroxy)methyl)-N-methoxy-N-methyl-1-((2-(trimethylsilyl)ethoxy)methyl)-1H-pyrazole-4-carboxamide (Int-9A). MS (ESI) [M+H]⁺: m/z 504, 506.

### Step B: 5-(2-bromo-3-chlorobenzyl)-N-methoxy-N-methyl-1H-pyrazole-4-carboxamide (Int-9B)

To a solution of 5-((2-bromo-3-chlorophenyl)(hydroxy)methyl)-N-methoxy-N-methyl-1-((2-(trimethylsilyl)ethoxy)methyl)-1H-pyrazole-4-carboxamide (Int-9A) (6.3 g, 12.48 mmol) in DCM (40 mL) was added triethylsilane (80 mL, 501 mmol) and TFA (40 mL, 519 mmol). The mixture was stirred at 60° C. for 16 h. The reaction mixture was cooled to room temperature and concentrated in vacuo. The residue was diluted with EtOAc (100 mL) and basified to pH ~8 with aq. 2 M NaOH. The organic layer was washed with brine (2×20 mL), dried with Na₂SO₄, filtered, and concentrated in vacuo. The crude residue was purified by flash silica gel chromatography (0 to 25% EtOAc in petroleum ether) to give 5-(2-bromo-3-chlorobenzyl)-N-methoxy-N-methyl-1H-pyrazole-4-carboxamide (Int-9B). MS (ESI) [M+H]⁺: m/z 358, 360.

### Step C: 5-chloro-2,9-dihydro-4H-benzo[f]indazol-4-one (Int-9C)

To a stirred solution of 5-(2-bromo-3-chlorobenzyl)-N-methoxy-N-methyl-1H-pyrazole-4-carboxamide (Int-9B) (1.2 g, 3.35 mmol) in THF (30 mL) was added isopropylmagnesium chloride lithium chloride complex (10.3 mL, 13.4 mmol, 1.3M in THF) at 0° C. The reaction was stirred at 0° C. for 2 h under N₂. The mixture was warmed to room temperature, diluted with EtOAc (50 mL), and quenched with 1 M aq. HCl to pH ~5. The reaction mixture was basified with sat. aq. NaHCO₃ and the organic layer was separated. The combined organic layer was washed with brine (20 mL), dried over Na₂SO₄, and concentrated in vacuo to give 5-chloro-2,9-dihydro-4H-benzo[f]indazol-4-one (Int-9C), which was used directly in the next step without further purification. MS (ESI) [M+H]⁺: m/z 219.

### Step D: 5-chloro-1-((trifluoromethyl)sulfonyl)-1H-benzo[f]indazol-4-yl trifluoromethanesulfonate (Int-9D)

To a solution of 5-chloro-2,9-dihydro-4H-benzo[f]indazol-4-one (Int-9C) (430 mg, 1.967 mmol) in DCM (20 mL) was added N,N-diisopropylethylamine (2.06 mL, 11.8 mmol) at −10° C. and the mixture was stirred at −10° C. for 10 min. Trifluoromethanesulfonic anhydride (1.99 mL, 11.8 mmol) was added to the above mixture, and the reaction was stirred at −10° C. for 1 h under N₂ atmosphere. The mixture was warmed to room temperature, diluted with DCM (30 mL), and washed with brine (10 mL). The organic layer was dried over Na₂SO₄, filtered, and concentrated in vacuo. The residue was purified by flash silica gel chromatography (petroleum ether) to give 5-chloro-1-((trifluoromethyl)sulfonyl)-1H-benzo[f]indazol-4-yl trifluoromethanesulfonate (Int-9D). ¹H NMR (500 MHz, CDCl₃) δ: 8.72 (s, 1H), 8.55 (s, 1H), 8.04 (d, J=8.5 Hz, 1H), 7.79 (dd, J=7.4, 1.0 Hz, 1H), 7.62 (dd, J=8.3, 7.6 Hz, 1H).

### Step E: 5-chloro-4-(4,4,5,5-tetramethyl-1,3,2-dioxaborolan-2-yl)-1-((trifluoromethyl)sulfonyl)-1H-benzo[f]indazole (Int-9)

In a glovebox, B₂pin₂ (1.90 g, 7.46 mmol), KOAc (0.732 g, 7.46 mmol), and Pd(dppf)Cl₂ (0.182 g, 0.249 mmol) was added to a solution of 5-chloro-1-((trifluoromethyl)sulfonyl)-1H-benzo[f]indazol-4-yl trifluoromethanesulfonate (Int-9D) (1.2 g, 2.49 mmol) in dioxane (20 mL). The mixture was heated to 90° C. and stirred for 4 h. After 4 h, the reaction mixture was cooled to room temperature, diluted with EtOAc (30 mL), and filtered. The filtrate was concentrated in vacuo and the residue was purified by flash silica gel chromatography (petroleum ether/EtOAc=20/1) to give 5-chloro-4-(4,4,5,5-tetramethyl-1,3,2-dioxaborolan-2-yl)-1-((trifluoromethyl)sulfonyl)-1H-benzo[f]indazole (Int-9). MS (ESI) [M+H]⁺: m/z 461.

The compound in the table below was synthesized using a similar procedure as described in the synthesis of Int-9 by making the appropriate substitutions for starting material, intermediates, and/or reagents. Appropriate substitutions are available commercially, synthesized as described in the literature, synthesized using methods available to those skilled in the art, or synthesized as described herein.

| | Starting | | | [M + H]⁺ |
|---|---|---|---|---|
| Int. | Material | Structure | Compound Name | Found |
| Int-10 | 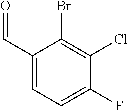 [SMILES: O=Cc1ccc(F)c(Cl)c1Br] | 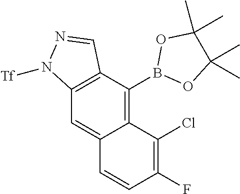 | 5-chloro-6-fluoro-4-(4,4,5,5- tetramethyl-1,3,2-dioxaborolan- 2-yl)-1- ((trifluoromethyl)sulfonyl)-1H- benzo[f]indazole | 479 |

### Intermediate 24: (6-fluoro-1-((trifluoromethyl)sulfonyl)-5-((triisopropylsilyl)ethynyl)-1H-benzo[f]indazol-4-yl)boronic acid (Int-24)

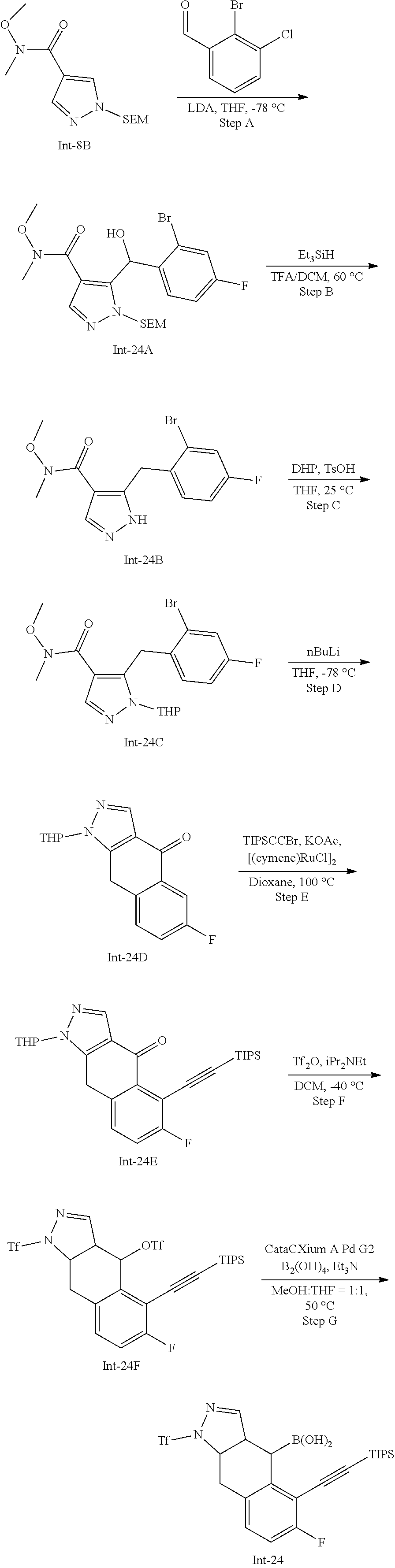

### Step A: 5-((2-bromo-4-fluorophenyl)(hydroxy)methyl)-N-methoxy-N-methyl-1-((2-(trimethylsilyl)ethoxy)methyl)-1H-pyrazole-4-carboxamide (Int-24A)

To a stirred solution of N-methoxy-N-methyl-1-((2-(trimethylsilyl)ethoxy)methyl)-1H-pyrazole-4-carboxamide (Int-8B) (24.7 g, 87.0 mmol) in THF (50 mL) was added lithium diisopropylamide (95 mL, 95 mmol, 1 M in THF) at −78° C. under N₂, and the reaction was stirred at −78° C. for 1 h. Then 2-bromo-4-fluorobenzaldehyde (16 g, 79 mmol) was added at −78° C., and the reaction was stirred at −78° C. for 1 h. The mixture was warmed to room temperature and concentrated in vacuo. The mixture was quenched with water (50 mL) and extracted with EtOAc (3×500 mL). The organic layers were washed with sat. brine (50 mL), dried over sodium sulfate, filtered, and concentrated in vacuo. The residue was purified by flash silica gel chromatography (0 to 25% EtOAc in petroleum ether) to give 5-((2-bromo-4-fluorophenyl)(hydroxy)methyl)-N-methoxy-N-methyl-1-((2-(trimethylsilyl)ethoxy)methyl)-1H-pyrazole-4-carboxamide (Int-24A). MS (ESI) [M+H]⁺: m/z 488, 490.

### Step B: 5-(2-bromo-4-fluorobenzyl)-N-methoxy-N-methyl-1H-pyrazole-4-carboxamide (Int-24B)

To a solution of 5-((2-bromo-4-fluorophenyl)(hydroxy)methyl)-N-methoxy-N-methyl-1-((2-(trimethylsilyl)ethoxy)methyl)-1H-pyrazole-4-carboxamide (Int-24A) (20.0 g, 40.9 mmol) in DCM (56 mL) was added triethylsilane (118 mL, 737 mmol) and TFA (56.8 mL, 737 mmol), and the mixture was stirred at 60° C. for 3 h. The reaction mixture was concentrated in vacuo and dissolved in EtOAc (400 mL). The reaction mixture was basified with sat. aq. NaHCO₃ to pH-8. The organic layer was washed with brine (2×30 mL), dried with sodium sulfate, filtered, and concentrated in vacuo. The residure was purified by flash silica gel chromatography (0 to 15% ethyl acetate in petroleum ether) to give 5-(2-bromo-4-fluorobenzyl)-N-methoxy-N-methyl-1H-pyrazole-4-carboxamide (Int-24B). MS (ESI) [M+H]⁺: m/z 342, 344.

### Step C: 5-(2-bromo-4-fluorobenzyl)-N-methoxy-N-methyl-1-(tetrahydro-2H-pyran-2-yl)-1H-pyrazole-4-carboxamide (Int-24C)

To a solution of 5-(2-bromo-4-fluorobenzyl)-N-methoxy-N-methyl-1H-pyrazole-4-carboxamide (Int-24B) (7.50 g, 21.9 mmol) in THF (80 mL) was added 4-methylbenzenesulfonic acid (0.377 g, 2.19 mmol) and DHP (4.01 mL, 43.8 mmol) at 25° C., and the mixture was stirred for 2 h. The mixture was concentrated in vacuo and the residue was purified by flash silica gel chromatography (petroleum ether/EtOAc=2/1) to give 5-(2-bromo-4-fluorobenzyl)-N-methoxy-N-methyl-1-(tetrahydro-2H-pyran-2-yl)-1H-pyrazole-4-carboxamide (Int-24C). MS (ESI) [M+H]⁺: m/z 426, 428.

### Step D: 6-fluoro-1-(tetrahydro-2H-pyran-2-yl)-1,3a-dihydro-4H-benzo[f]indazol-4-one (Int-24D)

To a stirred solution of 5-(2-bromo-4-fluorobenzyl)-N-methoxy-N-methyl-1-(tetrahydro-2H-pyran-2-yl)-1H-pyrazole-4-carboxamide (Int-24C) (22 g, 51.6 mmol) in THF (220 mL) was added n-BuLi (31 mL, 77 mmol, 2.5 M in hexane) dropwise over 30 min at −78° C. under N₂. After the addition was finished, the reaction was stirred at −78° C. for 0.5 h. The reaction mixture was poured into sat. aq. NH₄Cl (200 mL) and diluted with EtOAc (200 mL). The organic layer was separated, and the aqueous layer was extracted with EtOAc (3×200 mL). The combined organic layers were washed with brine (200 mL), dried over sodium sulfate, filtered, and concentrated in vacuo. The material was purified by flash silica gel chromatography (0 to 22% THF in petroleum ether) to give 6-fluoro-1-(tetrahydro-2H-pyran-2-yl)-1,3a-dihydro-4H-benzo[f]indazol-4-one (Int-24D). MS (ESI) [M+H]⁺: m/z 287.

### Step E: 6-fluoro-1-(tetrahydro-2H-pyran-2-yl)-5-((triisopropylsilyl)ethynyl)-1,9-dihydro-4H-benzo[f]indazol-4-one (Int-24E)

To a solution of 6-fluoro-1-(tetrahydro-2H-pyran-2-yl)-1,3a-dihydro-4H-benzo[f]indazol-4-one (Int-24D) (6.50 g, 22.7 mmol) in dioxane (120 mL) was added (bromoethynyl)triisopropylsilane (17.8 g, 68.1 mmol), potassium acetate (7.8 g, 79 mmol), and dichloro(p-cymene)ruthenium(II) dimer (6.95 g, 11.4 mmol) at 25° C. under N₂. The mixture was stirred at 100° C. for 3 h. The mixture was cooled, filtered, and the filtrate was concentrated in vacuo. The residue was purified by flash silica gel chromatography (2% ethyl acetate in DCM) to give 6-fluoro-1-(tetrahydro-2H-pyran-2-yl)-5-((triisopropylsilyl)ethynyl)-1,9-dihydro-4H-benzo[f]indazol-4-one (Int-24E). MS (ESI) [M+H]⁺: m/z 467.

### Step F: 6-fluoro-1-((trifluoromethyl)sulfonyl)-5-((triisopropylsilyl)ethynyl)-1H-benzo[f]indazol-4-yl trifluoromethanesulfonate (Int-24F)

A solution of 6-fluoro-1-(tetrahydro-2H-pyran-2-yl)-5-((triisopropylsilyl)ethynyl)-1,9-dihydro-4H-benzo[f]indazol-4-one (Int-24E) (5.5 g, 11.79 mmol) and N,N-diisopropylethylamine (12.4 mL, 70.7 mmol) in DCM (60 mL) was added Tf₂O (5.97 mL, 35.4 mmol) at −40° C., and the reaction mixture was stirred for 15 min. The reaction mixture was concentrated in vacuo at 30° C., and the residue was purified by flash silica gel chromatography (petroleum ether/EtOAc=20/1) to give 6-fluoro-1-((trifluoromethyl)sulfonyl)-5-((triisopropylsilyl)ethynyl)-1H-benzo[f]indazol-4-yl trifluoromethanesulfonate (Int-24F). ¹H NMR (400 MHz, CDCl₃) δ 8.71 (s, 1H), 8.50 (s, 1H), 8.04 (dd, J 5.36, 9.18 Hz, 1H), 7.55 (t, J 8.64 Hz, 1H), 1.17-1.24 (m, 21H).

### Step G: (6-fluoro-1-((trifluoromethyl)sulfonyl)-5-((triisopropylsilyl)ethynyl)-1H-benzo[f]indazol-4-yl)boronic acid (Int-24)

To a solution of 6-fluoro-1-((trifluoromethyl)sulfonyl)-5-((triisopropylsilyl)ethynyl)-1H-benzo[f]indazol-4-yl trifluoromethanesulfonate (Int-24F) (3.20 g, 4.95 mmol), hypodiboric acid (3.11 g, 34.6 mmol), and CataCXium A Pd G2 (0.265 g, 0.396 mmol) in MeOH (40 mL) and THF (40 mL) was added triethylamine (2.07 mL, 14.9 mmol) at 25° C., and the mixture was stirred at 50° C. for 1 h under N₂. The reaction mixture was concentrated in vacuo and purified by flash silica gel chromatography (0 to 10% EtOAc in petroleum ether) to give (6-fluoro-1-((trifluoromethyl)sulfonyl)-5-((triisopropylsilyl)ethynyl)-1H-benzo[f]indazol-4-yl)boronic acid (Int-24). MS (ESI) [M+H]⁺: m/z 543.

The compound in the table below was synthesized using a similar procedure as described in the synthesis of Int-24 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | Starting | | | [M + H]⁺ |
|---|---|---|---|---|
| Int. | Material | Structure | Compound Name | Found |
| Int-25 | 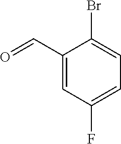 | 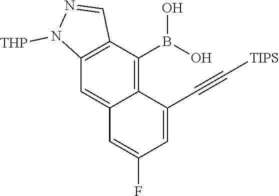 | (7-fluoro-1-(tetrahydro-2H- pyran-2-yl)-5- ((triisopropylsilyl)ethynyl)- 1H-benzo[f]indazol-4- yl)boronic acid | 495 |

### Intermediate 26: 4-(5,5-dimethyl-1,3,2-dioxaborinan-2-yl)-6-fluoro-1-((trifluoromethyl)sulfonyl)-5-((triisopropylsilyl)ethynyl)-1H-benzo[f]indazole (Int-26)

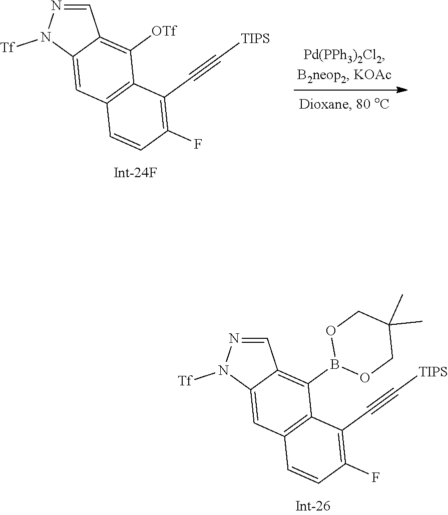

To a solution of 6-fluoro-1-((trifluoromethyl)sulfonyl)-5-((triisopropylsilyl)ethynyl)-1H-benzo[f]indazol-4-yl trifluoromethanesulfonate (Int-24F) (10 g, 15.5 mmol), potassium acetate (4.55 g, 46.4 mmol) and bis(neopentyl glycolato)diboron (10.5 g, 46.4 mmol) in dioxane (100 mL) was stirred at 25° C. for 10 min. Dichlorobis(triphenylphosphine)palladium(II) (1.09 g, 1.55 mmol) was added, and the mixture was stirred at 80° C. for 2 h under N₂ atmosphere. The mixture was warmed to rt and concentrated under reduced pressure. The residue was diluted with MeCN (30 mL), filtered, and the solid was collected and dried under vacuum to give 4-(5,5-dimethyl-1,3,2-dioxaborinan-2-yl)-6-fluoro-1-((trifluoromethyl)sulfonyl)-5-((triisopropylsilyl)ethynyl)-1H-benzo[f]indazole (Int-26). MS (ESI) [M+H]⁺: m/z 611.

### Intermediate 11: 3-((6-(bis(4-methoxybenzyl)amino)-4-methyl-3-(trifluoromethyl)pyridin-2-yl)sulfonyl)propanenitrile (Int-11)

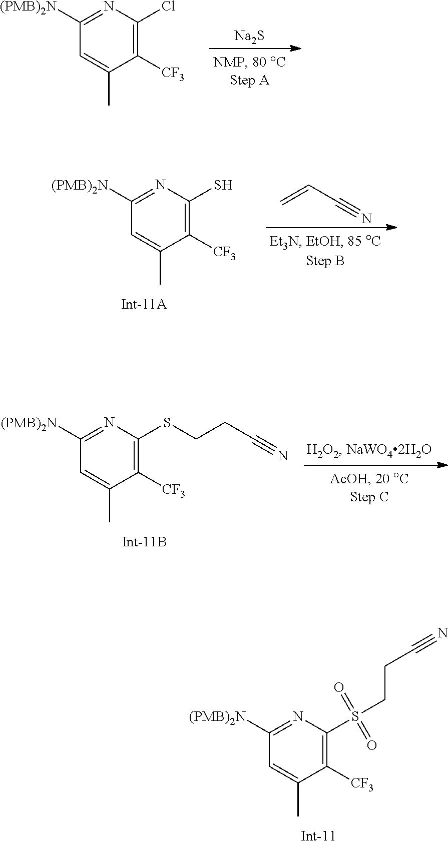

### Step A: 6-(bis(4-methoxybenzyl)amino)-4-methyl-3-(trifluoromethyl)pyridine-2-thiol (Int-11A)

To a solution of 6-chloro-N,N-bis(4-methoxybenzyl)-4-methyl-5-(trifluoromethyl)pyridin-2-amine (2.0 g, 4.44 mmol) in NMP (20 mL) was added Na₂S (2.08 g, 26.6 mmol) at 20° C. under N₂ atmosphere. The mixture was stirred at 80° C. for 12 h. The mixture was filtered and the filtrate was purified by reverse-phase MPLC directly (0 to 100% MeCN in H₂O w/TFA modifier) to give 6-(bis(4-methoxybenzyl)amino)-4-methyl-3-(trifluoromethyl)pyridine-2-thiol (Int-11A). MS (ESI) [M+H]⁺: m/z 449.

### Step B: 3-((6-(bis(4-methoxybenzyl)amino)-4-methyl-3-(trifluoromethyl)pyridin-2-yl)thio)propanenitrile (Int-11B)

To a solution of 6-(bis(4-methoxybenzyl)amino)-4-methyl-3-(trifluoromethyl)pyridine-2-thiol (Int-11A) (200 mg, 0.45 mmol) in EtOH (2 mL) was added acrylonitrile (30 mg, 0.57 mmol) and triethylamine (135 mg, 1.34 mmol) at 20° C. under N₂ atmosphere. The mixture was stirred at 85° C. for 3 h. The mixture was diluted with water (5 mL) and extracted with EtOAc (3×10 mL), dried over sodium sulfate, filtered, and the solvent was evaporated under reduced pressure. The product was purified by flash silica gel chromatography (0 to 30% EtOAc in petroleum ether) to give 3-((6-(bis(4-methoxybenzyl)amino)-4-methyl-3-(trifluoromethyl)pyridin-2-yl)thio)propanenitrile (Int-11B). MS (ESI) [M+H]⁺: m/z 502.

### Step C: 3-((6-(bis(4-methoxybenzyl)amino)-4-methyl-3-(trifluoromethyl)pyridin-2-yl)sulfonyl)propanenitrile (Int-11)

To a solution of 3-((6-(bis(4-methoxybenzyl)amino)-4-methyl-3-(trifluoromethyl)pyridin-2-yl)thio)propanenitrile (Int-11B) (169 mg, 0.337 mmol) in AcOH (2 mL) was added H₂O₂ (0.145 mL, 1.69 mmol, 30% in H₂O) and sodium tungstate dihydrate (11.11 mg, 0.034 mmol) at 20° C. The mixture was stirred at 20° C. for 12 h. The mixture was diluted with aq. Na₂SO₃ (2 mL) and evaporated under reduced pressure. The mixture was diluted with water (2 mL) and extracted with EtOAc (3×5 mL), dried over sodium sulfate, filtered, and the solvent was evaporated under reduced pressure. The residue was purified by preparative TLC (petroleum ether:EtOAc=2:1) to give 3-((6-(bis(4-methoxybenzyl)amino)-4-methyl-3-(trifluoromethyl)pyridin-2-yl)sulfonyl)propanenitrile (Int-11). MS (ESI) [M+H]⁺: m/z 534.

### Intermediate 12: 3-(bis(4-methoxyphenyl)amino)-7-fluoro-8-((triisopropylsilyl)ethynyl)isoquinolin-1-yl trifluoromethanesulfonate (Int-12)

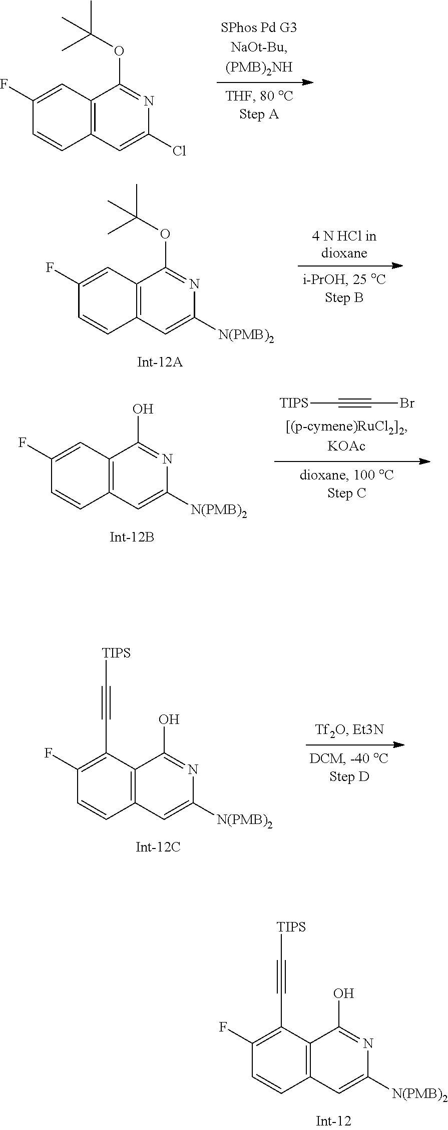

### Step A: 1-(tert-butoxy)-7-fluoro-N,N-bis(4-methoxybenzyl)isoquinolin-3-amine (Int-12A)

A mixture of 1-(tert-butoxy)-3-chloro-7-fluoroisoquinoline (4.70 g, 18.5 mmol), bis(4-methoxybenzyl)amine (7.15 g, 27.8 mmol), sodium tert-butoxide (3.56 g, 37.1 mmol), and SPhos Pd G3 (1.45 g, 1.85 mmol) in THF (80 mL) was degassed and backfilled with N₂ three times. The reaction mixture was stirred at 80° C. for 12 h under N₂. The mixture was cooled to room temperature, diluted with EtOAc (100 mL), and filtered. The filtrate was concentrated under reduced pressure, and the residue was purified by flash silica gel chromatography (0 to 5% EtOAc in petroleum ether) to give 1-(tert-butoxy)-7-fluoro-N,N-bis(4-methoxybenzyl)isoquinolin-3-amine (Int-12A). MS (ESI) [M+H]⁺: m/z 475.

### Step B: 3-(bis(4-methoxybenzyl)amino)-7-fluoroisoquinolin-1-ol (Int-12B)

To a solution of 1-(tert-butoxy)-7-fluoro-N,N-bis(4-methoxybenzyl)isoquinolin-3-amine (Int-12A) (2.0 g, 4.21 mmol) in i-PrOH (10 mL) was added HCl (10 mL, 4N in dioxane). The reaction mixture was stirred at 25° C. for 4 h. The reaction mixture was concentrated under reduced pressure. The crude mixture was diluted with EtOAc (20 mL) and sat. aq. NaHCO₃ (15 mL). The aqueous layer was separated and extracted with EtOAc (3×20 mL). The combined organic layers were dried over Na₂SO₄ and filtered. The filtrate was concentrated under reduced pressure and purified via flash silica gel chromatography (0 to 36% EtOAc in petroleum ether) to give 3-(bis(4-methoxybenzyl)amino)-7-fluoroisoquinolin-1-ol (Int-12B). MS (ESI) [M+H]⁺: m/z 419.

### Step C: 3-(bis(4-methoxybenzyl)amino)-7-fluoro-8-((triisopropylsilyl)ethynyl)isoquinolin-1-ol (Int-12C)

A mixture of 3-(bis(4-methoxybenzyl)amino)-7-fluoroisoquinolin-1-ol (Int-12B) (1.0 g, 2.39 mmol), (bromoethynyl)triisopropylsilane (1.0 g, 3.82 mmol), potassium acetate (0.47 g, 4.8 mmol), and dichloro(p-cymene)ruthenium(II) dimer (0.15 g, 0.24 mmol) in dioxane (15 mL) was degassed and backfilled with N₂ three times. The reaction mixture was stirred at 100° C. for 15 h under N₂. The reaction was filtered and concentrated under reduced pressure to give a crude reaction mixture which was purified by flash silica gel chromatography (0 to 23% EtOAc in petroleum ether) to give 3-(bis(4-methoxybenzyl)amino)-7-fluoro-8-((triisopropylsilyl)ethynyl)isoquinolin-1-ol (Int-12C). MS (ESI) [M+H]⁺: m/z 599.

### Step D: 3-(bis(4-methoxybenzyl)amino)-7-fluoro-8-((triisopropylsilyl)ethynyl)isoquinolin-1-yl trifluoromethanesulfonate (Int-12)

To a solution of 3-(bis(4-methoxybenzyl)amino)-7-fluoro-8-((triisopropylsilyl)ethynyl)isoquinolin-1-ol (Int-12C) (820 mg, 1.37 mmol) in DCM (15 mL) were added TEA (1.15 mL, 8.22 mmol) and Tf₂O (0.694 mL, 4.11 mmol) at −40° C. under N₂. The reaction mixture was stirred at −40° C. for 20 min. The reaction was concentrated under reduced pressure to give a residue which was purified by flash silica gel chromatography (0 to 32% EtOAc in petroleum ether) to give 3-(bis(4-methoxybenzyl)amino)-7-fluoro-8-((triisopropylsilyl)ethynyl)isoquinolin-1-yl trifluoromethanesulfonate (Int-12). MS (ESI) [M+H]⁺: m/z 731.

### Intermediate 13: (2-(bis(2,4-dimethoxybenzyl)amino)-5,7-difluorobenzo[d]oxazol-4-yl)boronic acid (Int-13)

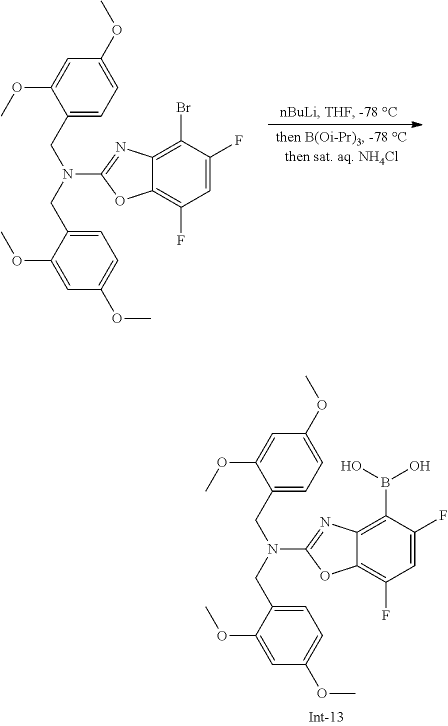 [SMILES: COc1ccc(CN(Cc2ccc(OC)cc2OC)c2nc3c(Br)c(F)cc(F)c3o2)c(OC)c1 ; COc1ccc(CN(Cc2ccc(OC)cc2OC)c2nc3c(B(O)O)c(F)cc(F)c3o2)c(OC)c1]

To a solution of 4-bromo-5,7-difluoro-N,N-bis(2,4-dimethoxybenzyl)benzo[d]oxazol-2-amine (0.41 g, 0.74 mmol) in THF (8 mL) was added n-butyllithium (0.6 mL, 0.961 mmol, 1.6M in hexanes) at −78° C. The resulting mixture was stirred at this temperature for 30 min. Then, triisopropyl borate (618 μL, 2.68 mmol) was added via syringe. The mixture was stirred at −78° C. for 10 min. Then, the mixture was quenched with sat. aq. NH₄Cl (2 mL) and extracted with EtOAc (3×20 mL). The organic layer was dried over anhydrous Na₂SO₄, filtered, and concentrated in vacuo to yield (2-(bis(2,4-dimethoxybenzyl)amino)-5,7-difluorobenzo[d]oxazol-4-yl)boronic acid (Int-13), which was used without further purification. MS (ESI) [M+H]⁺: m/z 515.

### Intermediate 14: ((2-fluoro-6-(methoxymethoxy)-8-(4,4,5,5-tetramethyl-1,3,2-dioxaborolan-2-yl)naphthalen-1-yl)ethynyl)triisopropylsilane (Int-14)

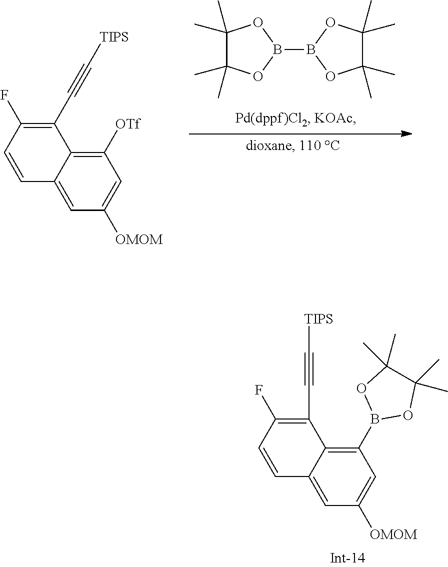

To a solution of 7-fluoro-3-(methoxymethoxy)-8-((triisopropylsilyl)ethynyl)naphthalen-1-yl trifluoromethanesulfonate (15 g, 28.1 mmol) in dioxane (150 mL) was added bis(pinacolato)diboron (13.3 g, 52.5 mmol), [1,1′-bis(diphenylphosphino)ferrocene]dichloropalladium(II) (2.053 g, 2.81 mmol), and potassium acetate (6.88 g, 70.1 mmol). The reaction was stirred at 110° C. under N₂ for 15 h. The mixture was concentrated and the residue was purified by flash silica gel chromatography (petroleum ether) to give ((2-fluoro-6-(methoxymethoxy)-8-(4,4,5,5-tetramethyl-1,3,2-dioxaborolan-2-yl)naphthalen-1-yl)ethynyl)triisopropylsilane (Int-14). ¹H NMR (400 MHz, CDCl₃) δ 7.69-7.64 (m, 1H), 7.51 (d, J=2.5 Hz, 1H), 7.38 (d, J=2.6 Hz, 1H), 7.23 (t, J=8.8 Hz, 1H), 5.29-5.26 (m, 2H), 3.51 (s, 3H), 1.44 (s, 12H), 1.18-1.14 (m, 21H).

### Intermediate 27: (S,Z)-(2-(fluoromethylene)tetrahydro-1H-pyrrolizin-7a(5H)-yl)methanol (Int-27)

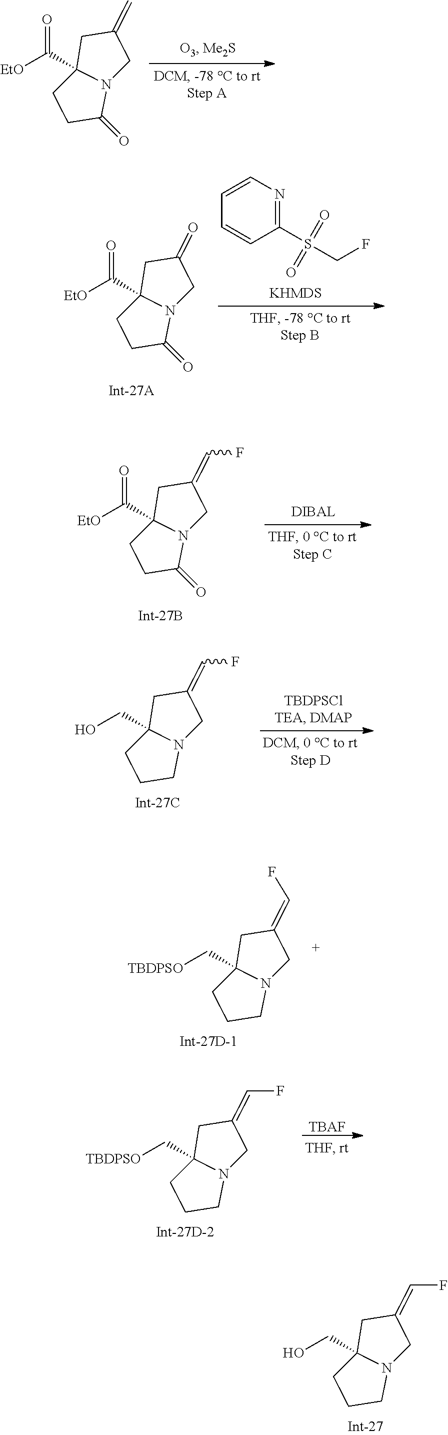

### Step A: Ethyl (S)-2,5-dioxotetrahydro-1H-pyrrolizine-7a(5H)-carboxylate (Int-27A)

A solution of ethyl (S)-2-methylene-5-oxotetrahydro-1H-pyrrolizine-7a(5H)-carboxylate (1.0 g, 4.8 mmol) in DCM (15 mL) was bubbled with O₃ at −78° C. until the mixture turned blue. Dimethylsulfide (0.594 g, 9.56 mmol) was added to the mixture, and the reaction was stirred at 25° C. for 16 h. The reaction mixture was washed with brine (20 mL), dried over sodium sulfate, filtered, and concentrated under reduced pressure. The product was purified by flash silica gel chromatography (0 to 50% EtOAc in petroleum ether) to give ethyl (S)-2,5-dioxotetrahydro-1H-pyrrolizine-7a(5H)-carboxylate (Int-27A). MS (ESI) [M+H]⁺: m/z 212.

### Step B: Ethyl (S)-2-(fluoromethylene)-5-oxotetrahydro-1H-pyrrolizine-7a(5H)-carboxylate (Int-27B)

To a solution of fluoromethyl 2-pyridyl sulfone (684 mg, 3.91 mmol) in THF (10 mL) was added KHMDS (4.51 mL, 4.51 mmol, 1 M in THF) at −78° C. under N₂. After 30 min, ethyl (S)-2,5-dioxotetrahydro-1H-pyrrolizine-7a(5H)-carboxylate (Int-27A) (750 mg, 3.55 mmol) in THF (8 mL) was added slowly at −78° C., and the mixture was stirred for 3 h. The reaction was warmed to room temperature and stirred for 1 h. The reaction was quenched with sat. aq. NH₄Cl (1 mL) and 3 M HCl (2 mL). The mixture was extracted with EtOAc (3×20 mL). The organic layer was dried over sodium sulfate, filtered, and the solvent was evaporated under reduced pressure. The product was purified by flash silica gel chromatography (0 to 30% EtOAc in petroleum ether) to give ethyl (S)-2-(fluoromethylene)-5-oxotetrahydro-TH-pyrrolizine-7a(5H)-carboxylate (Int-27B). MS (ESI) [M+H]⁺: m/z 228.

### Step C: (S)-(2-(fluoromethylene)tetrahydro-1H-pyrrolizin-7a(5H)-yl)methanol (Int-27C)

To a solution of ethyl (S)-2-(fluoromethylene)-5-oxotetrahydro-TH-pyrrolizine-7a(5H)-carboxylate (Int-27B) (200 mg, 0.880 mmol) in THF (4 mL) at 0° C. was added diisobutylaluminium hydride (8.80 mL, 8.80 mmol, 1 M in toluene) at 0° C., and the mixture was stirred for 1 h. The reaction was warmed to room temperature and stirred for 0.5 h. The reaction mixture was quenched with solid Na₂SO₄·10H₂O. The mixture was filtered, and the filtrate was concentrated under reduced pressure. The residue was purified by preparative TLC (DCM/MeOH=10/1) to give (S)-(2-(fluoromethylene)tetrahydro-1H-pyrrolizin-7a(5H)-yl)methanol (Int-27C). MS (ESI) [M+H]⁺: m/z 172.

### Step D: (S,E)-7a-(((tert-butyldiphenylsilyl)oxy)methyl)-2-(fluoromethylene)hexahydro-1H-pyrrolizine (Int-27D-1) & (S,Z)-7a-(((tert-butyldiphenylsilyl)oxy)methyl)-2-(fluoromethylene)hexahydro-1H-pyrrolizine (Int-27D-2)

To a mixture of (S)-(2-(fluoromethylene)tetrahydro-1H-pyrrolizin-7a(5H)-yl)methanol (Int-27C) (120 mg, 0.701 mmol) in DCM (5 mL) was added DMAP (8.6 mg, 0.070 mmol), Et₃N (0.195 mL, 1.40 mmol), and tert-butylchlorodiphenylsilane (289 mg, 1.05 mmol) at 0° C. under N₂, and the mixture was stirred at rt for 16 h. The mixture was diluted with water (5 mL) and extracted with DCM (3×20 mL). The combined organic layers were washed with water (10 mL) and brine (10 mL), dried over sodium sulfate, filtered, and concentrated under reduced pressure. The residue was purified by flash silica gel chromatography (0 to 50% EtOAc in petroleum ether) to give (S,E)-7a-(((tert-butyldiphenylsilyl)oxy)methyl)-2-(fluoromethylene)hexahydro-TH-pyrrolizine (Int-27D-1, the first peak eluting isomer, MS (ESI) [M+H]⁺: m/z 410) and (S,Z)-7a-(((tert-butyldiphenylsilyl)oxy)methyl)-2-(fluoromethylene)hexahydro-TH-pyrrolizine (Int-27D-2, the second peak eluting isomer, MS (ESI) [M+H]⁺: m/z 410).

### Step E: (S,Z)-(2-(fluoromethylene)tetrahydro-1H-pyrrolizin-7a(5H)-yl)methanol (Int-27)

To a mixture of (S,Z)-7a-(((tert-butyldiphenylsilyl)oxy)methyl)-2-(fluoromethylene)hexahydro-1H-pyrrolizine (Int-27D-2) (91 mg, 0.22 mmol) in THF (1 mL) was added TBAF (0.444 mL, 0.444 mmol, 1 M in THF) at rt under N₂, and the mixture was stirred at 25° C. for 16 h. The mixture was diluted with water (10 mL) and extracted with EtOAc (3×20 mL). The organic layer was dried over sodium sulfate, filtered, and the filtrate was concentrated under reduced. The residue was purified by preparative TLC (DCM/MeOH=10:1) to give (S,Z)-(2-(fluoromethylene)tetrahydro-1H-pyrrolizin-7a(5H)-yl)methanol (Int-27). MS (ESI) [M+H]⁺: m/z 172.

### Intermediate 15: (2,6-dioxabicyclo[3.2.1]octan-5-yl)methanol (Int-15)

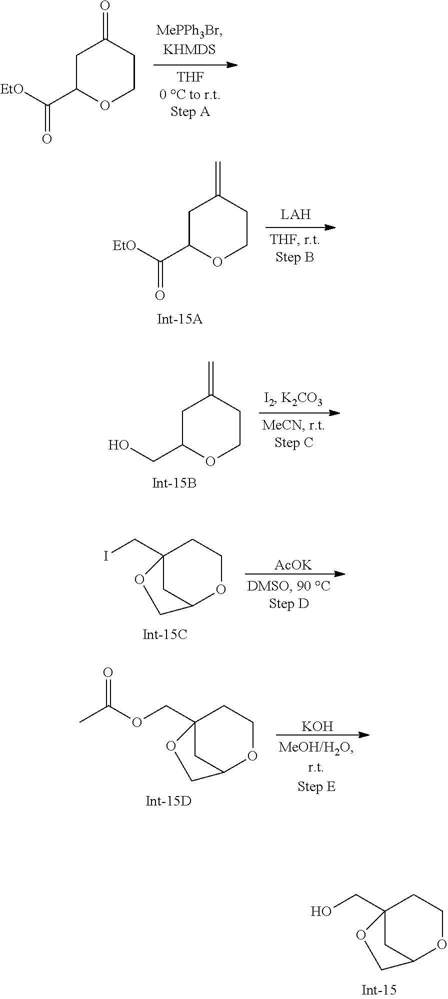

### Step A: Ethyl 4-methylenetetrahydro-2H-pyran-2-carboxylate (Int-15A)

To a stirred solution of methyltriphenylphosphonium bromide (2.49 g, 6.97 mmol) in THF (20 mL) was added potassium bis(trimethylsilyl)amide (6.97 mL, 6.97 mmol, 1M in THF) at 0° C. under N₂ atmosphere and the reaction was stirred at 0° C. for 0.5 h. Ethyl 4-oxotetrahydro-2H-pyran-2-carboxylate (1.00 g, 5.81 mmol) was added at 0° C. The reaction was warmed to room temperature and stirred at 25° C. for 2 h. The mixture was quenched with sat. aq. NH₄Cl (14 mL) and extracted with EtOAc (3×14 mL). The organic layers were washed with sat. aq. NaCl (30 mL), dried over Na₂SO₄, filtered, and concentrated in vacuo. The residue was purified by flash silica gel chromatography (0 to 30% EtOAc in petroleum ether) to give ethyl 4-methylenetetrahydro-2H-pyran-2-carboxylate (Int-15A). ¹H NMR (400 MHz, CDCl₃) δ 4.84 (br d, J=5.8 Hz, 2H), 4.28-4.24 (m, 2H), 4.23-4.16 (m, 1H), 4.01-3.93 (m, 1H), 3.52-3.42 (m, 1H), 2.58 (br d, J=13.4 Hz, 1H), 2.42-2.29 (m, 2H), 2.26-2.15 (m, 1H), 1.33-1.29 (m, 3H).

### Step B: (4-methylenetetrahydro-2H-pyran-2-yl)methanol (Int-15B)

To a stirred solution of ethyl 4-methylenetetrahydro-2H-pyran-2-carboxylate (Int-15A) (500 mg, 2.94 mmol) in THF (10 mL) was added lithium aluminum hydride (2.35 mL, 5.88 mmol, 2.5 M in THF) at 0° C. under N₂ atmosphere. The reaction was warmed to room temperature and stirred for 1 h. The mixture was quenched with Na₂SO₄ 10H₂O and filtered. The filter cake was washed with EtOAc and the filtrate was concentrated in vacuo to give (4-methylenetetrahydro-2H-pyran-2-yl)methanol (Int-15B) which was used directly in the next step without further purification. ¹H NMR (400 MHz, CDCl₃) δ 4.77 (dd, J=2.0, 4.2 Hz, 2H), 4.12 (dd, J=5.7, 10.9 Hz, 1H), 3.70-3.62 (m, 1H), 3.60-3.53 (m, 1H), 3.48-3.38 (m, 2H), 2.38-2.27 (m, 1H), 2.23-2.09 (m, 3H), 2.09-1.94 (m, 1H).

### Step C: 5-(iodomethyl)-2,6-dioxabicyclo[3.2.1]octane (Int-15C)

To a stirred solution of (4-methylenetetrahydro-2H-pyran-2-yl)methanol (Int-15B) (320 mg, 2.50 mmol) in MeCN (4 mL) was added K₂CO₃ (1.04 g, 7.49 mmol) and 12 (1.90 g, 7.49 mmol) at room temperature. The reaction was stirred at room temperature for 2 h. The mixture was quenched with aq. Na₂SO₃ (4 mL) and extracted with EtOAc (3×4 mL). The organic layers were washed with sat. aq. NaCl (10 mL), dried over Na₂SO₄, filtered, and concentrated in vacuo to give 5-(iodomethyl)-2,6-dioxabicyclo[3.2.1]octane (Int-15C) which was used directly in the next step without further purification. ¹H NMR (400 MHz, CDCl₃) δ 4.56 (br s, 1H), 4.23 (d, J=9.9 Hz, 1H), 3.99-3.88 (m, 3H), 3.46-3.35 (m, 2H), 1.90-1.80 (m, 3H), 1.75-1.67 (m, 1H).

### Step D: (2,6-dioxabicyclo[3.2.1]octan-5-yl)methyl acetate (Int-15D)

To a stirred solution of 5-(iodomethyl)-2,6-dioxabicyclo[3.2.1]octane (Int-15C) (580 mg, 2.28 mmol) in DMSO (10 mL) was added potassium acetate (896 mg, 9.13 mmol) at 0° C. under N₂ atmosphere. The reaction mixture was heated to 90° C. and stirred for 14 h. The mixture was cooled to room temperature and quenched with water (14 mL) and washed with EtOAc (3×10 mL). The combined organic layers were washed with sat. aq. NaCl (20 mL), dried over Na₂SO₄, filtered, and concentrated in vacuo. The crude residue was purified by flash silica gel chromatography (0 to 30% EtOAc in petroleum ether) to give (2,6-dioxabicyclo[3.2.1]octan-5-yl)methyl acetate (Int-15D). ¹H NMR (400 MHz, CDCl₃) δ 4.53 (br s, 1H), 4.30-4.20 (m, 2H), 4.11 (d, J=11.8 Hz, 1H), 3.98-3.89 (m, 3H), 2.11 (s, 3H), 1.84-1.81 (m, 1H), 1.75-1.69 (m, 2H), 1.66-1.59 (m, 1H).

### Step E: (2,6-dioxabicyclo[3.2.1]octan-5-yl)methanol (Int-15)

To a stirred solution of (2,6-dioxabicyclo[3.2.1]octan-5-yl)methyl acetate (Int-15D) (300 mg, 1.61 mmol) in MeOH (3 mL) and water (0.5 mL) was added KOH (452 mg, 8.06 mmol) and the reaction was stirred at 25° C. for 0.5 h. The mixture was diluted with water (4 mL) and extracted with EtOAc (3×4 mL). The organic layers were washed with sat. aq. NaCl (10 mL), dried over Na₂SO₄, filtered, and concentrated in vacuo to give (2,6-dioxabicyclo[3.2.1]octan-5-yl)methanol (Int-15) which was used without further purification. ¹H NMR (400 MHz, CDCl₃) δ 4.54 (br s, 1H), 4.26-4.18 (m, 1H), 4.00-3.87 (m, 3H), 3.75-3.67 (m, 1H), 3.64-3.56 (m, 1H), 1.91-1.86 (m, 1H), 1.73 (d, J=11.4 Hz, 1H), 1.70-1.62 (m, 1H), 1.59-1.50 (m, 1H).

### Intermediate 16: (8-((benzyloxy)methoxy)bicyclo[3.2.1]octan-1-yl)methanol (Int-16)

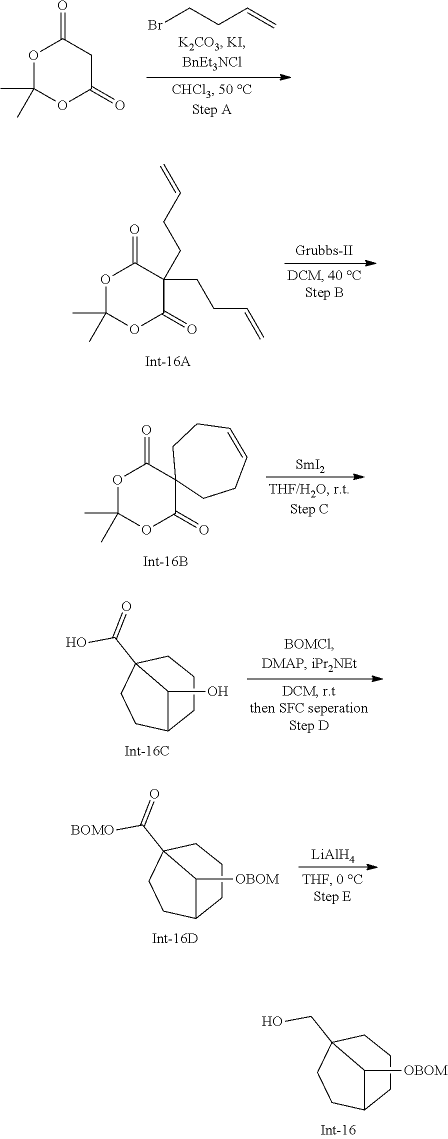

### Step A: 5,5-di(but-3-en-1-yl)-2,2-dimethyl-1,3-dioxane-4,6-dione (Int-16A)

To a solution of 2,2-dimethyl-1,3-dioxane-4,6-dione (11 g, 76 mmol) in CHCl₃ (110 mL) was added 4-bromo-1-butene (23.2 mL, 229 mmol), benzyltriethylammonium chloride (52.2 g, 229 mmol), K₂CO₃ (31.6 g, 229 mmol), and KI (2.53 g, 15.3 mmol) at 25° C. The mixture was heated to 50° C. and stirred for 6 h. After 6 h, the reaction was cooled to room temperature, diluted with EtOAc (200 mL), and filtrated. The filtrate was concentrated in vacuo and the residue was purified by flash silica gel chromatography (petroleum ether/EtOAc=10/1), followed by preparative reverse-phase HPLC (MeCN/H₂O w/NH₄HCO₃ modifier) to give 5,5-di(but-3-en-1-yl)-2,2-dimethyl-1,3-dioxane-4,6-dione (Int-16A). ¹H NMR (400 MHz, CDCl₃) δ 5.73-5.58 (m, 2H), 5.02-4.89 (m, 4H), 2.10-1.98 (m, 8H), 1.68 (s, 6H).

### Step B: 3,3-dimethyl-2,4-dioxaspiro[5.6]dodec-9-ene-1,5-dione (Int-16B)

To a solution of 5,5-di(but-3-en-1-yl)-2,2-dimethyl-1,3-dioxane-4,6-dione (Int-16A) (260 mg, 1.03 mmol) in DCM (10 mL) was added Grubbs-II (4.37 mg, 5.15 μmol) at 25° C. The mixture was heated to 40° C. and stirred for 2 h. The mixture was concentrated in vacuo and the residue was purified by flash silica gel chromatography (petroleum ether/EtOAc=10/1) to give 3,3-dimethyl-2,4-dioxaspiro[5.6]dodec-9-ene-1,5-dione (Int-16B). ¹H NMR (400 MHz, CDCl₃) δ 5.61 (t, J=2.7 Hz, 2H), 2.47 (br dd, J=2.6, 5.3 Hz, 4H), 2.34-2.21 (m, 4H), 1.66 (s, 6H).

### Step C: 8-hydroxybicyclo[3.2.1]octane-1-carboxylic acid (Int-16C)

A solution of 3,3-dimethyl-2,4-dioxaspiro[5.6]dodec-9-ene-1,5-dione (Int-16B) (1.00 g, 4.46 mmol) in THF (80 mL) and distilled water (80 mL) was purged with N₂ six times. Samarium(II) iodide (401 mL, 40.1 mmol, 0.1M in THF) was added to this solution at room temperature over 1 h under N₂ atmosphere and the mixture was stirred for 16 h. The reaction mixture was quenched with brine (60 mL) and tartaric acid (500 mg). The mixture was extracted with EtOAc (3×300 mL) and washed with brine (3×20 mL). The organic phase was dried over anhydrous Na₂SO₄, filtered, and concentrated in vacuo. The residue was purified by flash silica gel chromatography (petroleum ether/EtOAc=2/1) to give 8-hydroxybicyclo[3.2.1]octane-1-carboxylic acid (Int-16C). ¹H NMR (400 MHz, CDCl₃) δ 4.09 (d, J=5.1 Hz, 1H), 2.20-2.00 (m, 2H), 1.96-1.64 (m, 4H), 1.63-1.39 (m, 4H), 1.19 (br t, J=7.1 Hz, 1H).

### Step D: (Benzyloxy)methyl 8-((benzyloxy)methoxy)bicyclo[3.2.1]octane-1-carboxylate (Int-16D)

To a solution of 8-hydroxybicyclo[3.2.1]octane-1-carboxylic acid (Int-16C) (400 mg, 2.35 mmol) and DMAP (57.4 mg, 0.47 mmol) in DCM (8 mL) was added N,N-diisopropylethylamine (2.05 mL, 11.8 mmol) and ((chloromethoxy)methyl)benzene (0.981 mL, 7.05 mmol) at 0° C. The mixture was warmed to room temperature and stirred for 16 h. After 16 h, the reaction mixture was concentrated in vacuo and purified by flash silica gel chromatography (petroleum ether/EtOAc=5/1). The racemic material was separated by preparative SFC (Column A, 35% MeOH with 0.1% NH₄OH) to afford (benzyloxy)methyl 8-((benzyloxy)methoxy)bicyclo[3.2.1]octane-1-carboxylate (Int-16D, the second eluting isomer from SFC). ¹H NMR (400 MHz, CDCl₃) δ 7.28-7.22 (m, 10H), 5.33-5.27 (m, 1H), 5.22 (s, 1H), 4.80-4.70 (m, 2H), 4.58 (s, 2H), 4.54 (s, 2H), 4.19 (d, J=5.0 Hz, 1H), 2.08-1.96 (m, 1H), 1.93-1.73 (m, 3H), 1.72-1.64 (m, 1H), 1.63-1.39 (m, 5H), 1.24-1.11 (m, 1H).

### Step E: (8-((benzyloxy)methoxy)bicyclo[3.2.1]octan-1-yl)methanol (Int-16)

To a solution of (benzyloxy)methyl 8-((benzyloxy)methoxy)bicyclo[3.2.1]octane-1-carboxylate (Int-16D) (170 mg, 0.414 mmol) in THF (5 mL) was added LiAlH₄ (0.331 mL, 0.828 mmol, 2.5 M in THF) at 0° C., and the mixture was stirred at 0° C. for 1 h. The reaction mixture was quenched with Na₂SO₄·10H₂O and diluted with EtOAc (30 mL). The resulting mixture was dried over anhydrous Na₂SO₄ and the mixture was filtered. The filtrate was concentrated in vacuo and was purified by preparative TLC (petroleum ether/EtOAc=5/1) to give (8-((benzyloxy)methoxy)bicyclo[3.2.1]octan-1-yl)methanol (Int-16). ¹H NMR (400 MHz, CDCl₃) δ 7.40-7.37 (m, 4H), 7.36-7.30 (m, 1H), 4.86 (s, 2H), 4.75-4.68 (m, 1H), 4.66-4.61 (m, 1H), 3.81 (d, J=4.9 Hz, 1H), 3.52-3.41 (m, 2H), 2.29 (br d, J=4.6 Hz, 1H), 1.94-1.77 (m, 2H), 1.74-1.64 (m, 1H), 1.63-1.57 (m, 3H), 1.53-1.49 (m, 1H), 1.48-1.39 (m, 1H), 1.34-1.23 (m, 1H), 1.18-1.05 (m, 1H).

### Intermediate 28: ((6′R,7a′S)-6′-fluorotetrahydrospiro[cyclopropane-1,3′-pyrrolizin]-7a′(5′H)-yl)methanol (Int-28)

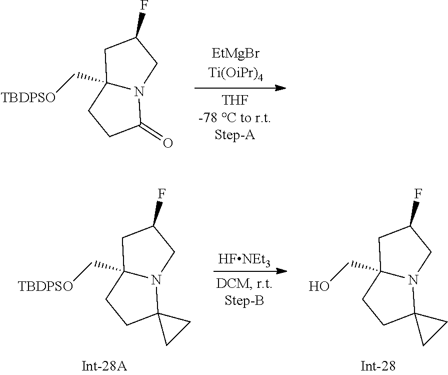

### Step A: (6′R,7a′S)-7a′-(((tert-butyldiphenylsilyl)oxy)methyl)-6′-fluorohexahydrospiro[cyclopropane-1,3′-pyrrolizine] (Int-28A)

To a solution of titanium(IV) isopropoxide (1.12 mL, 3.69 mmol) in THF (8.00 mL) was added ethylmagnesium bromide (7.39 mL, 7.39 mmol, 1.00 M in THF) dropwise at −78° C. After stirring for 10 min at −78° C., a solution of (6R,7aS)-7a-(((tert-butyldiphenylsilyl)oxy)methyl)-6-fluorohexahydro-3H-pyrrolizin-3-one (760 mg, 1.85 mmol) in THF (2.4 mL) was added dropwise. The reaction was stirred at −78° C. for 10 min and at room temperature for 100 min. The reaction was quenched by addition of sat. aq. NaHCO₃ (20 mL) and water (10 mL). The biphasic mixture was extracted with EtOAc (3×30 mL), and the combined organic phases were washed with brine (50 mL), dried over sodium sulfate, filtered, and concentrated. Purification by column chromatography (EtOAc in DCM, 0% to 40%) afforded (6′R,7a′S)-7a′-(((tert-butyldiphenylsilyl)oxy)methyl)-6′-fluorohexahydrospiro[cyclopropane-1,3′-pyrrolizine](Int-28A). MS (ESI) [M+H]⁺: m/z 424.

### Step B: ((6′R,7a′S)-6′-fluorotetrahydrospiro[cyclopropane-1,3′-pyrrolizin]-7a′(5′H)-yl)methanol (Int-28)

To a solution of (6′R,7a′S)-7a′-(((tert-butyldiphenylsilyl)oxy)methyl)-6′-fluorohexahydrospiro[cyclopropane-1,3′-pyrrolizine] (Int-28A) (300 mg, 708 μmol) in DCM (7.00 mL) was added triethylamine trihydrofluoride (231 μL, 1.42 mmol). The reaction was stirred at room temperature for 1.5 h. The mixture was concentrated under reduced pressure, and the residue was dissolved in DCM (3 mL) and extracted with 1 M aq. HCl (3×3 mL). The combined aqueous phases were lyophylized to afford ((6′R,7a′S)-6′-fluorotetrahydrospiro[cyclopropane-1,3′-pyrrolizin]-7a′(5′H)-yl)methanol (Int-28), isolated as the HCl salt. MS (ESI) [M+H]⁺: m/z 186.

### Intermediate 29: (R)-(2,2-difluoro-1-((6-fluoro-2-azaspiro[3.3]heptan-2-yl)methyl)cyclopropyl)methanol (Int-29)

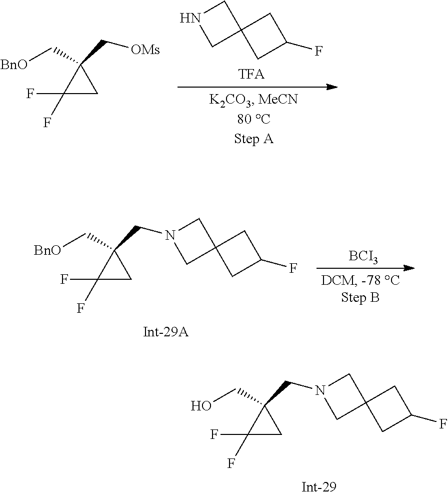 [SMILES: CS(=O)(=O)OC[C@@]1(COCc2ccccc2)CC1(F)F ; FC1CC2(CNC2)C1 ; FC1CC2(C1)CN(C[C@@]1(COCc3ccccc3)CC1(F)F)C2 ; "BCI3" ; OC[C@]1(CN2CC3(CC(F)C3)C2)CC1(F)F]

### Step A: (R)-2-((1-((benzyloxy)methyl)-2,2-difluorocyclopropyl)methyl)-6-fluoro-2-azaspiro[3.3]heptane (Int-29A)

To a solution of (S)-(1-((benzyloxy)methyl)-2,2-difluorocyclopropyl)methyl methanesulfonate (300 mg, 0.979 mmol) in MeCN (4 mL) was added potassium carbonate (406 mg, 2.94 mmol), 6-fluoro-2-azaspiro[3.3]heptane, TFA (337 mg, 1.47 mmol) at 25° C. The mixture was stirred at 80° C. for 3 h. The mixture was cooled, filtered, and the solvent was evaporated under reduced pressure. The product was purified by flash silica gel chromatography (0~30% DCM/MeOH) to give (R)-2-((1-((benzyloxy)methyl)-2,2-difluorocyclopropyl)methyl)-6-fluoro-2-azaspiro[3.3]heptane (Int-29A). MS (ESI) [M+H]⁺: m/z 326.

### Step B: (R)-(2,2-difluoro-1-((6-fluoro-2-azaspiro[3.3]heptan-2-yl)methyl)cyclopropyl)methanol (Int-29)

To a solution of (R)-2-((1-((benzyloxy)methyl)-2,2-difluorocyclopropyl)methyl)-6-fluoro-2-azaspiro[3.3]heptane (Int-29A) (200 mg, 0.615 mmol) in DCM (2 mL) was added boron trichloride solution (4.92 mL, 4.92 mmol, 1 M in DCM) at −78° C. under N₂ atmosphere. The mixture was stirred at −78° C. for 1 h. The mixture was quenched with MeOH (3 mL) at −78° C., warmed to rt, basified with NH₃ (7 M in MeOH, adjusted to pH>7), and the mixture was concentrated under reduced pressure. The residue was purified by preparative TLC (DCM:MeOH=10:1) to give (R)-(2,2-difluoro-1-((6-fluoro-2-azaspiro[3.3]heptan-2-yl)methyl)cyclopropyl)methanol (Int-29). MS (ESI) [M+H]⁺: m/z 236.

The examples in the table below were synthesized using a similar procedure as described in the synthesis of Int-29 by making the appropriate substitutions for starting material, intermediates, and/or reagents. Appropriate substitutions are available commercially, synthesized as described in the literature, synthesized using methods available to those skilled in the art, or synthesized as described herein.

| | Starting | | | [M + H]⁺ | |
|---|---|---|---|---|---|
| Int. | material | Structure | Compound Name | Found | Conditions |
| Int- 30 | 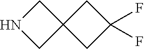 | 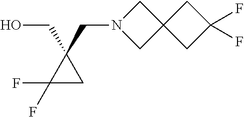 [SMILES: OC[C@]1(CN2CC3(C2)CC(F)(F)C3)CC1(F)F] | (R)-(1-((6,6-difluoro- 2-azaspiro[3.3]heptan- 2-yl)methyl)-2,2- difluorocyclopro- pyl)methanol | 254 | Step B: DCM (0.35 M), Iron (III) chloride (3 equiv), 50 °C, 2 h |
| Int- 31 | 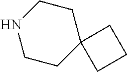 | 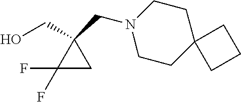 [SMILES: OC[C@]1(CN2CCC3(CCC3)CC2)CC1(F)F] | (R)-(1-((7- azaspiro[3.5]nonan-7- yl)methyl)-2,2- difluorocyclopro- pyl)methanol | 246 | Step B: Pd/C, 16 psi, H₂, trifluoroethano, rt, 3 h |
| Int- 32 | 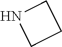 | 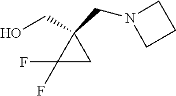 [SMILES: OC[C@]1(CN2CCC2)CC1(F)F] | (R)-(1-(azetidin-1- ylmethyl)-2,2- difluorocyclopro- pyl)methanol | 178 | Step B: Pd/C, 16 psi, H₂, trifluoroethanol, rt, 3 h |
| Int- 33 | 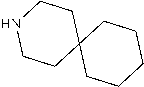 [SMILES: C1CCC2(CC1)CCNCC2] | 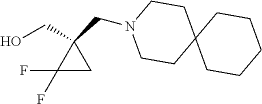 [SMILES: OC[C@]1(CN2CCC3(CCCCC3)CC2)CC1(F)F] | (R)-(1-((3- azaspiro[5.5]undecan- 3-yl)methyl)-2,2- difluorocyclopro- pyl)methanol | 274 | |
| Int- 34 | 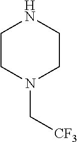 [SMILES: FC(F)(F)CN1CCNCC1] | 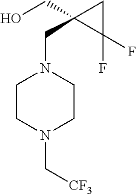 [SMILES: OC[C@]1(CN2CCN(CC(F)(F)F)CC2)CC1(F)F] | (R)-(2,2-difluoro-1- ((4-(2,2,2- trifluoroethyl)piper- azin-1- yl)methyl)cyclopropyl) methanol | 289 | |
| Int- 35 | 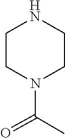 | 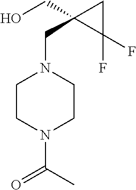 [SMILES: CC(=O)N1CCN(C[C@@]2(CO)CC2(F)F)CC1] | (R)-1-(4-((2,2- difluoro-1- (hydroxymethyl)cyclo- propyl)methyl)piper- azin-1-yl)ethan-1-one | 249 | |
| Int- 36 | 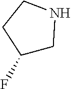 | 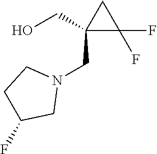 [SMILES: OC[C@]1(CN2CC[C@@H](F)C2)CC1(F)F] | ((R)-2,2-difluoro-1- (((R)-3- fluoropyrrolidin-1- yl)methyl)cyclopropyl) methanol | 210 | |
| Int- 37 | 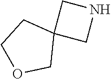 | 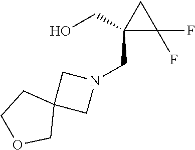 [SMILES: OC[C@]1(CN2CC3(CCOC3)C2)CC1(F)F] | (R)-(1-((6-oxa-2- azaspiro[3.4]octan-2- yl)methyl)-2,2- difluorocyclopro- pyl)methanol | 234 | |
| Int- 38 | 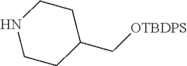 | 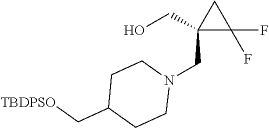 | (R)-(1-((4-(((tert- butyldiphenylsilyl)o- xy)methyl)piperidin-1- yl)methyl)-2,2- difluorocyclopro- pyl)methanol | 474 | Step B: Pd/C, 16 psi, H₂, trifluoroethanol, rt |

### Intermediate 39: (R)-(2,2-difluoro-1-((2-((1-methyl-1H-1,2,3-triazol-4-yl)methyl)-7-azaspiro[3.5]nonan-7-yl)methyl)cyclopropyl)methanol (Int-39)

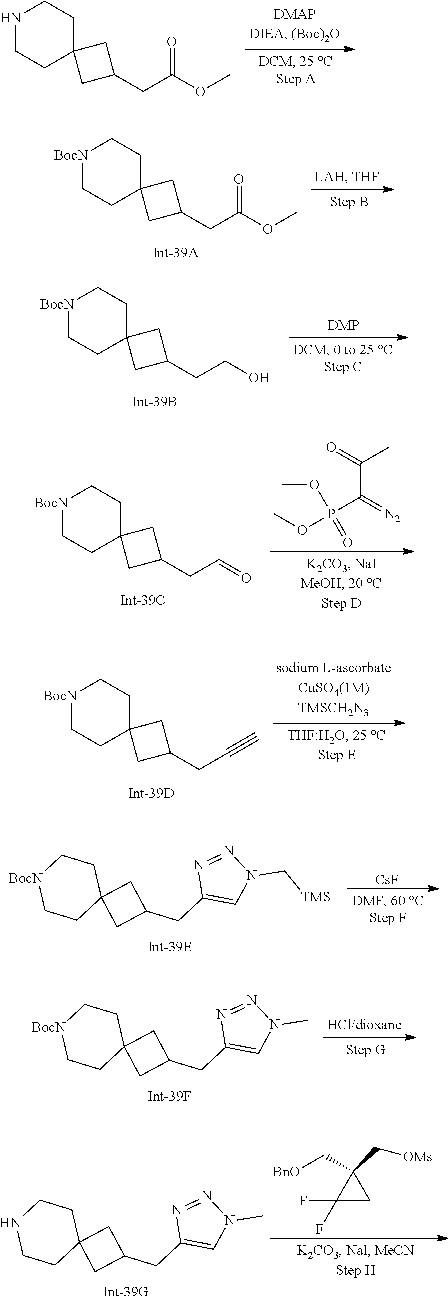

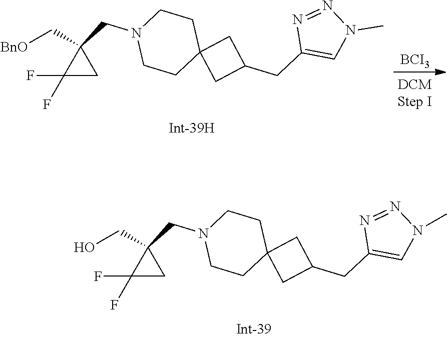

### Step A: tert-butyl 2-(2-methoxy-2-oxoethyl)-7-azaspiro[3.5]nonane-7-carboxylate (Int-39A)

To a solution of methyl 2-(7-azaspiro[3.5]nonan-2-yl)acetate (500 mg, 2.53 mmol) in DCM (5 mL) was added Boc₂O (0.883 mL, 3.80 mmol), DIEA (1.328 mL, 7.60 mmol) and DMAP (31.0 mg, 0.253 mmol) dropwise at 25° C. for 12 h. The mixture was diluted with water (5 mL) and extracted with DCM (3*10 mL). The organic layer was washed with brine (2*5 mL), dried over sodium sulfate, filtered, and the filtrate was concentrated under reduced pressure. The residue was purified by flash silica gel chromatography (0~15% ethyl acetate/petroleum ether) to give tert-butyl 2-(2-methoxy-2-oxoethyl)-7-azaspiro[3.5]nonane-7-carboxylate (Int-39A). MS (ESI) [M+H]⁺: m/z 295.

### Step B: tert-butyl 2-(2-hydroxyethyl)-7-azaspiro[3.5]nonane-7-carboxylate (Int-39B)

To a solution of tert-butyl 2-(2-methoxy-2-oxoethyl)-7-azaspiro[3.5]nonane-7-carboxylate (Int-39A) (600 mg, 2.018 mmol) in THF (7 mL) was added LiAlH₄ (1.614 mL, 4.04 mmol) (2.5 M in THF) at 0° C. The reaction was stirred at 0° C. for 1 h. The reaction mixture was quenched with Na₂SO₄ (10 g), EtOAc (10 mL) and H₂O (1 mL) at 0° C. in sequence until the bubble disappeared. The mixture was stirred at 25° C. for 0.5 h. The mixture was filtered, and the filtered cake was washed with EtOAc (30 mL). The mixture was concentrated under reduced pressure to give the product tert-butyl 2-(2-hydroxyethyl)-7-azaspiro[3.5]nonane-7-carboxylate (Int-39B), which was used without further purification. MS (ESI) [M-tBu+H]⁺: m/z 214.

### Step C: tert-butyl 2-(2-oxoethyl)-7-azaspiro[3.5]nonane-7-carboxylate (Int-39C)

To a solution of tert-butyl 2-(2-hydroxyethyl)-7-azaspiro[3.5]nonane-7-carboxylate (Int-39B) (500 mg, 1.856 mmol) in DCM (5 mL) was added 3-oxo-115-benzo[d][1,2]iodaoxole-1,1,1(3H)-triyl triacetate (1.57 g, 3.71 mmol) at 0° C. The mixture was stirred at 25° C. for 1 h. The mixture was quenched with aq. Na₂SO₃ (6 mL) and aq. NaHCO₃ (6 mL), extracted with DCM (3*15 mL). The organic layer was washed with brine (3*5 mL), dried over sodium sulfate, filtered, and the filtrate was concentrated under reduced pressure. The residue was purified by preparative TLC (petroleum ether:ethyl acetate=3:1) to give tert-butyl 2-(2-oxoethyl)-7-azaspiro[3.5]nonane-7-carboxylate (Int-39C). MS (ESI) [M+H]⁺: m/z 268.

### Step D: tert-butyl 2-(prop-2-yn-1-yl)-7-azaspiro[3.5]nonane-7-carboxylate (Int-39D)

To a solution of tert-butyl 2-(2-oxoethyl)-7-azaspiro[3.5]nonane-7-carboxylate (Int-39C) (190 mg, 0.711 mmol) in MeOH (2 mL) was added dimethyl (1-diazo-2-oxopropyl)phosphonate (164 mg, 0.853 mmol), sodium iodide (106 mg, 0.711 mmol) and K₂CO₃ (196 mg, 1.42 mmol) at 20° C. The mixture was stirred at 20° C. for 3 h. The mixture was filtered, and the filtrate was concentrated under reduced pressure. The residue was purified by preparative TLC (petroleum ether:ethyl acetate=5:1) to give tert-butyl 2-(prop-2-yn-1-yl)-7-azaspiro[3.5]nonane-7-carboxylate (Int-39D). MS (ESI) [M+H]⁺: m/z 264.

### Step E: tert-butyl 2-((1-((trimethylsilyl)methyl)-1H-1,2,3-triazol-4-yl)methyl)-7-azaspiro[3.5]nonane-7-carboxylate (Int-39E)

To a solution of tert-butyl 2-(prop-2-yn-1-yl)-7-azaspiro[3.5]nonane-7-carboxylate (Int-39D)(144 mg, 0.547 mmol) in THF:Water (3:1, 2 mL) was added copper (II) sulfate (0.055 mL, 0.055 mmol, 1 M in water), (azidomethyl)trimethylsilane (141 mg, 1.09 mmol) and sodium (R)-5-((S)-1,2-dihydroxyethyl)-4-hydroxy-2-oxo-2,5-dihydrofuran-3-olate (217 mg, 1.09 mmol) at 20° C. under N₂ atmosphere. The mixture was stirred at 25° C. for 1 h. The mixture was diluted with water (1 mL) and extracted with EtOAc (3*4 mL). The organic layer was washed with brine (2*2 mL), dried over sodium sulfate, filtered, and the filtrate was concentrated under reduced pressure to give tert-butyl 2-((1-((trimethylsilyl)methyl)-1H-1,2,3-triazol-4-yl)methyl)-7-azaspiro[3.5]nonane-7-carboxylate (Int-39E), which was used without further purification. MS (ESI) [M+H]⁺: m/z 393.

### Step F: tert-butyl 2-((1-methyl-1H-1,2,3-triazol-4-yl)methyl)-7-azaspiro[3.5]nonane-7-carboxylate (Int-39F)

To a solution of tert-butyl 2-((1-((trimethylsilyl)methyl)-1H-1,2,3-triazol-4-yl)methyl)-7-azaspiro[3.5]nonane-7-carboxylate (Int-39E) (215 mg, 0.548 mmol) in DMF (2 mL) was added CsF (333 mg, 2.190 mmol) at 20° C. The mixture was stirred at 60° C. for 1 h. The mixture was diluted with water (2 mL) and extracted with EtOAc (3*4 mL). The organic layer was washed with water (2*1 mL), dried over sodium sulfate, filtered, and the filtrate was concentrated under reduced pressure. The residue was purified by preparative TLC (petroleum ether:ethyl acetate=1:1) to give tert-butyl 2-((1-methyl-1H-1,2,3-triazol-4-yl)methyl)-7-azaspiro[3.5]nonane-7-carboxylate (Int-39F). MS (ESI) [M+H]⁺: m/z 321.

### Step G: 2-((1-methyl-1H-1,2,3-triazol-4-yl)methyl)-7-azaspiro[3.5]nonane (Int-39G)

A solution of tert-butyl 2-((1-methyl-1H-1,2,3-triazol-4-yl)methyl)-7-azaspiro[3.5]nonane-7-carboxylate (Int-39F) (130 mg, 0.406 mmol) in HCl (1 mL, 2 M in dioxane) was stirred at 20° C. for 4 h. The solvent was removed by nitrogen flow to give 2-((1-methyl-1H-1,2,3-triazol-4-yl)methyl)-7-azaspiro[3.5]nonane (Int-39G), which was used without further purification. MS (ESI) [M+H]⁺: m/z 221.

### Step H: (R)-7-((1-((benzyloxy)methyl)-2,2-difluorocyclopropyl)methyl)-2-((1-methyl-1H-1,2,3-triazol-4-yl)methyl)-7-azaspiro[3.5]nonane (Int-39H)

To a solution of 2-((1-methyl-1H-1,2,3-triazol-4-yl)methyl)-7-azaspiro[3.5]nonane (Int-39G) (130 mg, 0.590 mmol) in MeCN (5 mL) was added (S)-(1-((benzyloxy)methyl)-2,2-difluorocyclopropyl)methyl methanesulfonate (181 mg, 0.590 mmol), sodium iodide (88 mg, 0.590 mmol) and K₂CO₃ (245 mg, 1.770 mmol) at 25° C. under N₂ atmosphere. The mixture was stirred at 80° C. for 16 h. The mixture was diluted with water (5 mL) and extracted with EtOAc (20 mL*3). The organic layer was washed with brine (5 mL*3), dried over sodium sulfate, filtered, and the filtrate was concentrated under reduced pressure. The residue was purified by preparative TLC (DCM:MeOH=15:1) to give (R)-7-((1-((benzyloxy)methyl)-2,2-difluorocyclopropyl)methyl)-2-((1-methyl-1H-1,2,3-triazol-4-yl)methyl)-7-azaspiro[3.5]nonane (Int-39H). MS (ESI) [M+H]⁺: m/z 431.

### Step I: (R)-(2,2-difluoro-1-((2-((1-methyl-1H-1,2,3-triazol-4-yl)methyl)-7-azaspiro[3.5]nonan-7-yl)methyl)cyclopropyl)methanol (Int-39)

To a solution of (R)-7-((1-((benzyloxy)methyl)-2,2-difluorocyclopropyl)methyl)-2-((1-methyl-1H-1,2,3-triazol-4-yl)methyl)-7-azaspiro[3.5]nonane (Int-39H) (110 mg, 0.255 mmol) in DCM (3 mL) was added boron trichloride solution (2.04 mL, 2.04 mmol) (1 M in DCM) at −78° C. under N₂ atmosphere. The mixture was stirred at −78° C. for 1 h. Additional boron trichloride solution (2.04 mL, 2.04 mmol) was added to the mixture. The mixture was stirred at −78° C. for another 1 h. The mixture was quenched MeOH (2 mL) at −78° C., warmed to rt, and basified with NH₃ (7M in MeOH) until pH to 7. The mixture was concentrated under reduced pressure. The residue was purified by preparative TLC (DCM:NH₃ (7M in MeOH)=20:1) to give (R)-(2,2-difluoro-1-((2-((1-methyl-1H-1,2,3-triazol-4-yl)methyl)-7-azaspiro[3.5]nonan-7-yl)methyl)cyclopropyl)methanol (Int-39). MS (ESI) [M+H]⁺: m/z 341.

The example in the table below was synthesized using a similar procedure as described in the synthesis of Int-39 by making the appropriate substitutions for starting material, intermediates, and/or reagents. Appropriate substitutions are available commercially, synthesized as described in the literature, synthesized using methods available to those skilled in the art, or synthesized as described herein.

| | | | | [M + H]⁺ | |
|---|---|---|---|---|---|
| Ex. | Starting material | Structure | Compound Name | Found | Conditions |
| Int- 40 | 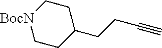 [SMILES: C#CCCC1CCN(C(=O)OC(C)(C)C)CC1] 1,1-4imethylethyl | 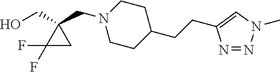 [SMILES: Cn1cc(CCC2CCN(C[C@@]3(CO)CC3(F)F)CC2)nn1] | (R)-(2,2-difluoro-1- ((4-(2-(1-methyl- 1H-1,2,3-triazol-4- yl)ethyl)piperidin- 1- | 315 | Start at Step E using shown starting material |
| | 4-(3-butyn-1-y1)-1- | | yl)methyl)cyclopro- | | |
| | piperidinecar- | | pyl)methanol | | |
| | boxylate | | | | |

### Intermediate 41: 1-((1R,5S)-3,8-diazabicyclo[3.2.1]octan-3-yl)propan-2-ol, HCl (Int-41-1 and Int-41-2)

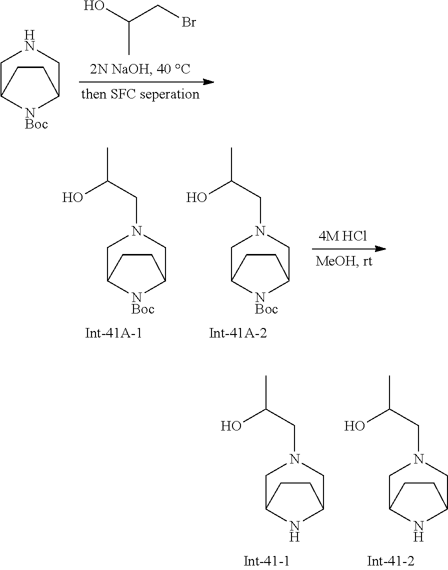

### Step A: tert-butyl (1R,5S)-3-(2-hydroxypropyl)-3,8-diazabicyclo[3.2.1]octane-8-carboxylate (Int-41A-1 and Int-41A-2)

A solution of 1-bromopropan-2-ol (851 mg, 6.12 mmol), tert-butyl (1R,5S)-3,8-diazabicyclo[3.2.1]octane-8-carboxylate (650 mg, 3.06 mmol) and 2 N NaOH (0.70 mL) were heated at 40° C. 2 h. The mixture was cooled to rt and extracted with Et₂O. The combined organic layers were washed with brine, dried over Na₂SO₄, filtered, and the fitlrate concentrated under reduced pressure. The product was purified by silica gel column (ELSD, 0-50% MeOH in DCM) to yield the racemic product. The isomers were separated by chiral SFC (20% IPA w/0.1% NH₄OH) to afford tert-butyl (1R,5S)-3-(2-hydroxypropyl)-3,8-diazabicyclo[3.2.1]octane-8-carboxylate (the first eluting isomer, Int-41A-1) and tert-butyl (1R,5S)-3-(2-hydroxypropyl)-3,8-diazabicyclo[3.2.1]octane-8-carboxylate (the second eluting isomer, Int-41A-2). MS (ESI) [M+H]⁺: m/z 271.

### Step B: 1-((1R,5S)-3,8-diazabicyclo[3.2.1]octan-3-yl)propan-2-ol, HCl (Int-41-1)

To a solution of tert-butyl (1R,5S)-3-(2-hydroxypropyl)-3,8-diazabicyclo[3.2.1]octane-8-carboxylate (Int-41A-1) (110 mg, 407 μmol) in methanol (0.7 mL) was added HCl (406 μL, 1.63 mmol, 4 M in dioxane). The reaction mixture was stirred at RT for 2 hrs and concentrated in vacuo to yield 1-((1R,5S)-3,8-diazabicyclo[3.2.1]octan-3-yl)propan-2-ol, HCl which was taken as such without further purifications (Int-41-1). MS (ESI) [M+H]⁺: m/z 171.

The same procedure was used to synthesize 1-((1R,5S)-3,8-diazabicyclo[3.2.1]octan-3-yl)propan-2-ol, HCl (Int-41-2), using the second eluting isomer (Int-41A-2) from Step A. MS (ESI) [M+H]⁺: m/z 171.

### Intermediate 42: 1-((1R,5S)-3,8-diazabicyclo[3.2.1]octan-8-yl)propan-2-ol, HCl (Int-42-1 and Int-42-2)

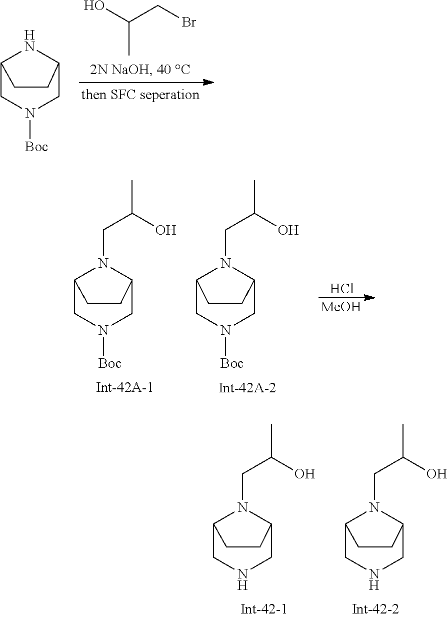

### Step A: tert-butyl (1R,5S)-8-(2-hydroxypropyl)-3,8-diazabicyclo[3.2.1]octane-3-carboxylate (Int-42A-1 and Int-42A-2)

To a solution of 1-bromopropan-2-ol (982 mg, 7.07 mmol) and tert-butyl (1R,5S)-3,8-diazabicyclo[3.2.1]octane-3-carboxylate (750 mg, 3.53 mmol) was added 2N NaOH (7.00 mL) and stirred at 40° C. for 4 h. The mixture was cooled to RT and extracted with ether. The combined organic layers were washed with saturated brine, dried over Na₂SO₄, filtered, and concentrated under reduced pressure. The product was purified by silica gel column chromatography (ELSD, 0-50% of MeOH/DCM) to get the racemic product. The material was separated by chiral SFC (15% MeOH w/0.1% NH₄OH) to yield tert-butyl (1R,5S)-8-(2-hydroxypropyl)-3,8-diazabicyclo[3.2.1]octane-3-carboxylate (first eluting isomer, Int-42A-1) and tert-butyl (1R,5S)-8-(2-hydroxypropyl)-3,8-diazabicyclo[3.2.1]octane-3-carboxylate (second eluting isomer, Int-42A-2). MS (ESI) [M+H]⁺: m/z 271.

### Step B: 1-((1R,5S)-3,8-diazabicyclo[3.2.1]octan-8-yl)propan-2-ol, HCl (Int-42-1)

To a solution of tert-butyl (1R,5S)-8-(2-hydroxypropyl)-3,8-diazabicyclo[3.2.1]octane-3-carboxylate (200 mg, 740 mol, Int-42A-1) in methanol (2.00 mL) was added HCl (740 μL, 2.96 mmol, 4 M in dioxane) and stirred at RT for 4 h. The mixture was concentrated under reduced pressure to yield 1-((1R,5S)-3,8-diazabicyclo[3.2.1]octan-8-yl)propan-2-ol, HCl (Int-42-1). MS (ESI) [M+H]⁺: m/z 171.

The same procedure was used to synthesize 1-((1R,5S)-3,8-diazabicyclo[3.2.1]octan-8-yl)propan-2-ol, HCl (Int-42-2), using the second eluting isomer (Int-42A-2) from Step A. MS (ESI) [M+H]⁺: m/z 171.

### Intermediate 17: tert-butyl (1R,5S)-3-(8-chloro-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate (Int-17)

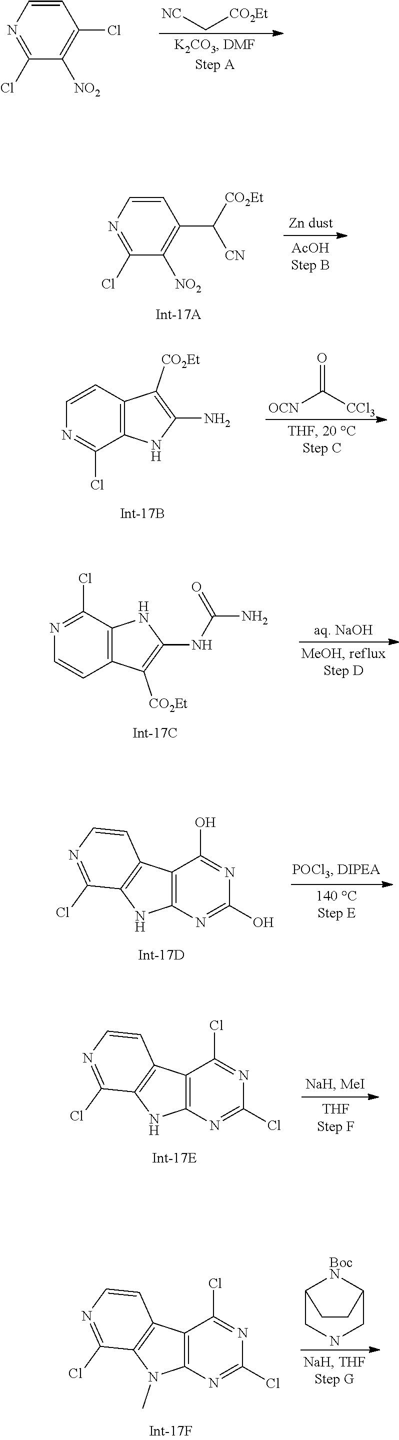

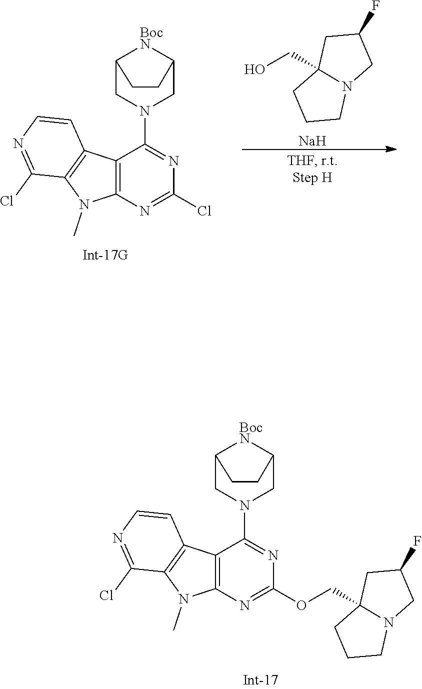

### Step A: Ethyl 2-(2-chloro-3-nitropyridin-4-yl)-2-cyanoacetate (Int-17A)

To a solution of 2,4-dichloro-3-nitropyridine (10 g, 51.8 mmol) in DMF (100 mL) was added K₂CO₃ (14.3 g, 104 mmol) and ethyl 2-cyanoacetate (6.15 g, 54.4 mmol) at 25° C. The mixture was stirred at 25° C. for 2 h. The reaction mixture was quenched with water (400 mL) and extracted with EtOAc (3×200 mL). The combined organic layers were washed with brine (4×50 mL), dried over Na₂SO₄, filtered, and the filtrate was concentrated under reduced pressure. The residue was purified by flash silica gel chromatography (10% EtOAc in petroleum ether) to give ethyl 2-(2-chloro-3-nitropyridin-4-yl)-2-cyanoacetate (Int-17A). MS (ESI) [M+H]⁺: m/z 270. ¹H NMR (400 MHz, DMSO-d₆) δ 8.15 (d, J=6.1 Hz, 1H), 7.95 (s, 1H), 7.80 (d, J=6.1 Hz, 1H), 3.98 (q, J=7.0 Hz, 2H), 1.15 (t, J=6.5 Hz, 3H).

### Step B: Ethyl 2-amino-7-chloro-1H-pyrrolo[2,3-c]pyridine-3-carboxylate (Int-17B)

To a solution of ethyl 2-(2-chloro-3-nitropyridin-4-yl)-2-cyanoacetate (Int-17A) (8 g, 29.7 mmol) in acetic acid (80 mL) was added zinc dust (15.1 g, 231 mmol) at 0° C. The reaction was warmed to 25° C. and stirred for 1 h. The reaction mixture was filtered, and the filtrate was concentrated under reduced pressure to give a residue which was dissolved in THF and filtered again. The filtrate was concentrated under reduced pressure and purified by flash silica gel chromatography (0 to 15% (7N NH₃ in MeOH) in DCM) to give ethyl 2-amino-7-chloro-1H-pyrrolo[2,3-c]pyridine-3-carboxylate (Int-17B). MS (ESI) [M+H]⁺: m/z 240.

### Step C: Ethyl 7-chloro-2-ureido-1H-pyrrolo[2,3-c]pyridine-3-carboxylate (Int-17C)

To a solution of ethyl 2-amino-7-chloro-1H-pyrrolo[2,3-c]pyridine-3-carboxylate (Int-17B) (4 g, 16.7 mmol) in THF (70 mL) was added 2,2,2-trichloroacetyl isocyanate (9.43 g, 50.1 mmol) at 20° C. The resulting mixture was stirred at 20° C. for 1 h. The solution was concentrated under reduced pressure to give a residue which was diluted with EtOAc (100 mL). The mixture was filtered and the solid was collected and dried in vacuo to give ethyl 7-chloro-2-ureido-1H-pyrrolo[2,3-c]pyridine-3-carboxylate (Int-17C). MS (ESI) [M+H]⁺: m/z 283. ¹H NMR (400 MHz, DMSO-d₆) δ 11.79 (s, 1H), 11.69 (s, 1H), 8.11 (d, J=5.36 Hz, 1H), 7.76 (d, J=5.36 Hz, 1H), 4.39 (q, J=7.03 Hz, 2H), 1.40 (t, J=7.09 Hz, 3H).

### Step D: 8-chloro-9H-pyrido[4′,3′:4,51]pyrrolo[2,3-d]pyrimidine-2,4-diol (Int-17D)

To a solution of ethyl 7-chloro-2-ureido-1H-pyrrolo[2,3-c]pyridine-3-carboxylate (Int-17C) (4 g, 14.2 mmol) in MeOH (40 mL) was added sodium hydroxide (50 mL, 75.0 mmol, 1.5M in H₂O). The reaction mixture was stirred at 90° C. for 16 h. The reaction mixture was concentrated under reduced pressure. The mixture was acidified with HCl (2 M) to pH 3, yellow solid was precipitated and the solid was filtered, collected, and dried in vacuo to give 8-chloro-9H-pyrido[4′,3′:4,5]pyrrolo[2,3-d]pyrimidine-2,4-diol (Int-17D) which was used directly in the next step without further purification. MS (ESI) [M+H]⁺: m/z 237. ¹H NMR (400 MHz, DMSO-d₆) δ 12.80 (br s, 1H), 12.10 (br s, 1H), 11.06 (s, 1H), 8.07 (d, J=5.14 Hz, 1H), 7.68 (d, J=5.38 Hz, 1H).

### Step E: 2,4,8-trichloro-9H-pyrido[4′,3′:4,5]pyrrolo[2,3-d]pyrimidine (Int-17E)

A solution of 8-chloro-9H-pyrido[4′,3′:4,5]pyrrolo[2,3-d]pyrimidine-2,4-diol (Int-17D) (1.5 g, 6.34 mmol), N,N-diisopropylethylamine (6.29 mL, 38.0 mmol) in phosphorus oxychloride (18.9 mL, 203 mmol) was stirred at 140° C. in a sealed tube for 48 h. The mixture was concentrated under reduced pressure to give a residue which was purified by flash silica gel chromatography (100% EtOAc) to give 2,4,8-trichloro-9H-pyrido[4′,3′:4,5]pyrrolo[2,3-d]pyrimidine (Int-17E). MS (ESI) [M+H]⁺: m/z 273.

### Step F: 2,4,8-trichloro-9-methyl-9H-pyrido[4′,3′:4,5]pyrrolo[2,3-d]pyrimidine (Int-17F)

To a solution of 2,4,8-trichloro-9H-pyrido[4′,3′:4,5]pyrrolo[2,3-d]pyrimidine (Int-17E) (2.7 g, 9.87 mmol) in THF (50 mL) was added NaH (1.19 g, 29.6 mmol, 60 wt % in mineral oil) followed by iodomethane (1.47 mL, 22.7 mmol). The reaction mixture was stirred at 30° C. for 16 h. The reaction was quenched with sat. aq. NH₄Cl (20 mL) and extracted with EtOAc (50 mL). The organic layer was washed with brine (20 mL) and concentrated in vacuo to afford 2,4,8-trichloro-9-methyl-9H-pyrido[4′,3′:4,5]pyrrolo[2,3-d]pyrimidine (Int-17F), which was used directly in the next step without further purification. MS (ESI) [M+H-Cl+MeO]⁺: m/z 283.

### Step G: Tert-butyl (1R,5S)-3-(2,8-dichloro-9-methyl-9H-pyrido[4′,3′:4,5]pyrrolo[2,3-d]pyrimidin-4-yl)-3,8-diazabicyclo[3.2.1]octane-8-carboxylate (Int-17G)

To a solution of 2,4,8-trichloro-9-methyl-9H-pyrido[4′,3′:4,5]pyrrolo[2,3-d]pyrimidine (Int-17F) (80 mg, 0.28 mmol) in THF (2 mL) was added NaH (22.3 mg, 0.556 mmol, 60 wt % in mineral oil) and tert-butyl (1R,5S)-3,8-diazabicyclo[3.2.1]octane-8-carboxylate (59 mg, 0.28 mmol). The reaction mixture was stirred at 25° C. for 2 h. The mixture was diluted with EtOAc (10 mL), washed with sat. aq. NH₄Cl (1 mL) and brine (1 mL), dried over Na₂SO₄, and filtered. The filtrate was concentrated under reduced pressure and purified by flash silica gel chromatography (30% EtOAc in hexanes) to give tert-butyl (1R,5S)-3-(2,8-dichloro-9-methyl-9H-pyrido[4′,3′:4,5]pyrrolo[2,3-d]pyrimidin-4-yl)-3,8-diazabicyclo[3.2.1]octane-8-carboxylate (Int-17G). MS (ESI) [M+H]⁺: m/z 463.

### Step H: Tert-butyl (1R,5S)-3-(8-chloro-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate (Int-17)

To a solution of tert-butyl (1R,5S)-3-(2,8-dichloro-9-methyl-9H-pyrido[4′,3′:4,5]pyrrolo[2,3-d]pyrimidin-4-yl)-3,8-diazabicyclo[3.2.1]octane-8-carboxylate (Int-17G) (100 mg, 0.22 mmol) and ((2R,7aS)-2-fluorotetrahydro-1H-pyrrolizin-7a(5H)-yl)methanol (34 mg, 0.22 mmol) in THF (2 mL) was added NaH (17 mg, 0.43 mmol, 60 wt % in mineral oil). The reaction mixture was stirred at 25° C. for 2 h. The mixture was diluted with EtOAc (10 mL) and washed with sat. aq. NH₄Cl solution (1 mL). The organic layer was washed with brine (1 mL), dried over Na₂SO₄, and filtered. The mixture was concentrated under reduced pressure and purified by preparative-scale TLC (SiO₂, DCM:MeOH=15:1) to give tert-butyl (1R,5S)-3-(8-chloro-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate (Int-17). MS (ESI) [M+H]⁺: m/z 586. ¹H NMR (400 MHz, MeOD) δ 8.15 (d, J=5.5 Hz, 1H), 7.73 (d, J=5.5 Hz, 1H), 5.55-5.73 (m, 1H), 4.62-4.73 (m, 2H), 4.51 (br t, J=10.6 Hz, 2H), 4.39 (br s, 2H), 4.21 (s, 3H), 3.92-4.11 (m, 3H), 3.65 (br dd, J=12.2, 5.9 Hz, 2H), 3.47-3.57 (m, 1H), 2.70-2.88 (m, 1H), 2.61-2.69 (m, 1H), 2.46-2.55 (m, 1H), 2.39 (dq, J=12.0, 6.1 Hz, 2H), 2.19-2.30 (m, 1H), 1.91 (br d, J=5.0 Hz, 2H), 1.76 (br d, J=8.7 Hz, 2H), 1.55 (s, 9H).

### Intermediate 18: 2-(((2R,7aS)-2-fluorotetrahydro-1H-pyrrolizin-7a(5H)-yl)methoxy)-9-methyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-ol (Int-18)

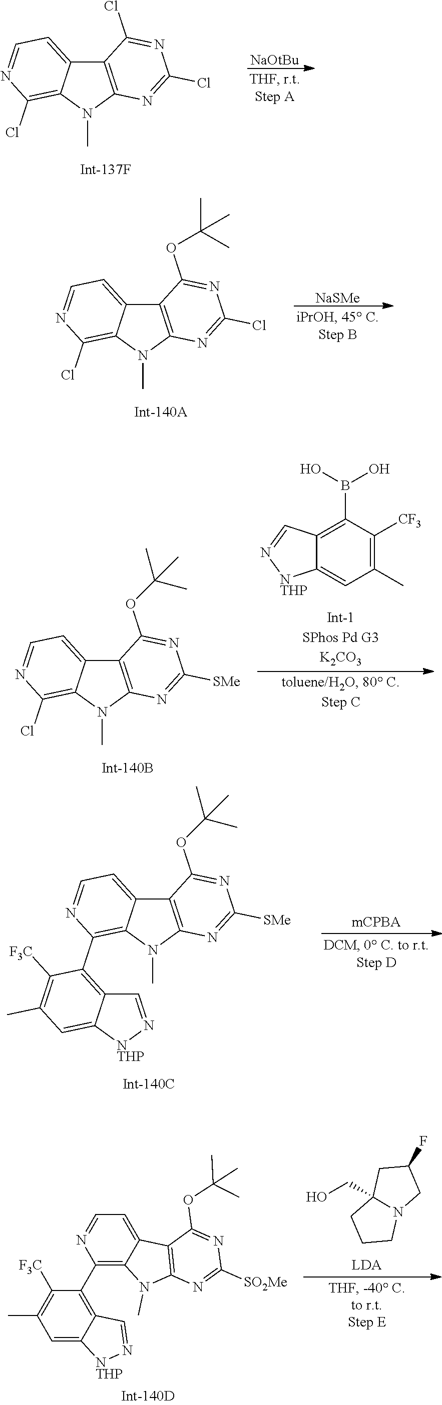

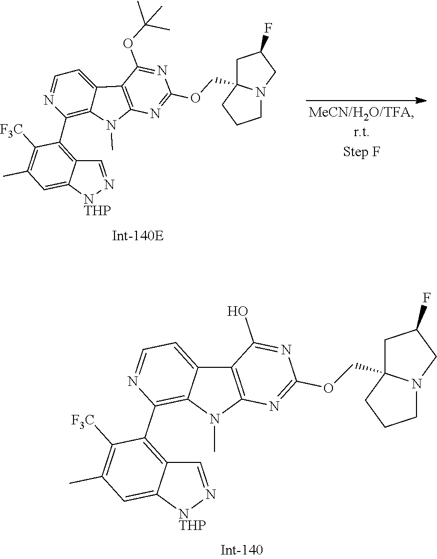

### Step A: 4-(tert-butoxy)-2,8-dichloro-9-methyl-9H-pyrido[4′,3′:4,5]pyrrolo[2,3-d]pyrimidine (Int-18A)

A flask with a stir bar was charged with 2,4,8-trichloro-9-methyl-9H-pyrido[4′,3′:4,5]pyrrolo[2,3-d]pyrimidine (Int-17F) (1.05 g, 3.65 mmol). The flask was fitted with a septum and placed under nitrogen. THF (18.3 mL) was added via syringe. Then sodium tert-butoxide (1.92 mL, 3.83 mmol, 2M in THF) was added dropwise. The reaction was stirred at room temperature for 2 h. After 2 h, sat. aq. NaHCO₃ (20 mL) was added and the reaction was diluted with EtOAc (30 mL). The layers were separated and the aqueous phase was extracted with EtOAc (2×30 mL). The combined organic layers were washed with brine (50 mL), dried over Na₂SO₄, filtered, and concentrated in vacuo. The material was purified via silica gel chromatography (0 to 4% (3:1 EtOAc:EtOH) in DCM) to obtain 4-(tert-butoxy)-2,8-dichloro-9-methyl-9H-pyrido[4′,3′:4,5]pyrrolo[2,3-d]pyrimidine (Int-18A). MS (ESI) [M+H]⁺: m/z 325.

### Step B: 4-(tert-butoxy)-8-chloro-9-methyl-2-(methylthio)-9H-pyrido[4′,3′:4,5]pyrrolo[2,3-d]pyrimidine (Int-18B)

A vial with a stir bar was charged with 4-(tert-butoxy)-2,8-dichloro-9-methyl-9H-pyrido[4′,3′:4,5]pyrrolo[2,3-d]pyrimidine (Int-18A) (434 mg, 1.34 mmol) and sodium thiomethoxide (140 mg, 2.0 mmol) and placed under nitrogen. 2-propanol (8.9 mL) was added via syringe and the reaction was stirred at 45° C. for 2 hours. The reaction was diluted with sat. aq. NH₄Cl (5 mL) and EtOAc (10 mL). The layers were separated and the aqueous phase was extracted with EtOAc (2×10 mL). The combined organic layers were washed with brine (20 mL), dried over Na₂SO₄, filtered, and concentrated in vacuo to yield 4-(tert-butoxy)-8-chloro-9-methyl-2-(methylthio)-9H-pyrido[4′,3′:4,5]pyrrolo[2,3-d]pyrimidine (Int-18B), which was used without further purification. MS (ESI) [M+H]⁺: m/z 337.

### Step C: 4-(tert-butoxy)-9-methyl-8-(6-methyl-1-(tetrahydro-2H-pyran-2-yl)-5-(trifluoromethyl)-1H-indazol-4-yl)-2-(methylthio)-9H-pyrido[4′,3′:4,5]pyrrolo[2,3-d]pyrimidine (Int-18C)

A mixture of 4-(tert-butoxy)-8-chloro-9-methyl-2-(methylthio)-9H-pyrido[4′,3′:4,5]pyrrolo[2,3-d]pyrimidine (Int-18B) (280 mg, 0.831 mmol), (6-methyl-1-(tetrahydro-2H-pyran-2-yl)-5-(trifluoromethyl)-1H-indazol-4-yl)boronic acid (Int-1) (545 mg, 1.66 mmol), SPhos Pd G3 (324 mg, 0.416 mmol), and K₂CO₃ (1.25 mL, 2.49 mmol, 2 M in H₂O) was suspended in toluene (11.9 mL). The mixture was sparged with nitrogen for 15 min. The reaction was stirred at 80° C. for 3 h. The reaction mixture was cooled to room temperature, diluted with EtOAc (20 mL), washed with brine (50 mL), dried over Na₂SO₄, filtered, and concentrated in vacuo. The residue was purified by silica gel chromatography (0 to 100% EtOAc in hexane) to provide 4-(tert-butoxy)-9-methyl-8-(6-methyl-1-(tetrahydro-2H-pyran-2-yl)-5-(trifluoromethyl)-1H-indazol-4-yl)-2-(methylthio)-9H-pyrido[4′,3′:4,5]pyrrolo[2,3-d]pyrimidine (Int-18C). MS (ESI) [M+H]⁺: m/z 585.

### Step D: 4-(tert-butoxy)-9-methyl-8-(6-methyl-1-(tetrahydro-2H-pyran-2-yl)-5-(trifluoromethyl)-1H-indazol-4-yl)-2-(methylsulfonyl)-9H-pyrido[4′,3′:4,5]pyrrolo[2,3-d]pyrimidine (Int-18D)

A vial with a stir bar was charged with 4-(tert-butoxy)-9-methyl-8-(6-methyl-1-(tetrahydro-2H-pyran-2-yl)-5-(trifluoromethyl)-1H-indazol-4-yl)-2-(methylthio)-9H-pyrido[4′,3′:4,5]pyrrolo[2,3-d]pyrimidine (Int-18C) (292 mg, 0.50 mmol). DCM (3.3 mL) was added, and the reaction was cooled to 0° C. mCPBA (215 mg, 1.25 mmol) was added in a single portion and the reaction was warmed to room temperature and stirred for 1.5 h. The reaction was diluted with DCM (5 mL) and washed with sat. aq. NaHCO₃ (5 mL) and water (3×5 mL). The organic phase was dried over Na₂SO₄, filtered, and concentrated in vacuo to yield 4-(tert-butoxy)-9-methyl-8-(6-methyl-1-(tetrahydro-2H-pyran-2-yl)-5-(trifluoromethyl)-1H-indazol-4-yl)-2-(methylsulfonyl)-9H-pyrido[4′,3′:4,5]pyrrolo[2,3-d]pyrimidine (Int-18D), which was used without further purification. MS (ESI) [M+H]⁺: m/z 617.

### Step E: 4-(tert-butoxy)-2-(((2R,7aS)-2-fluorotetrahydro-1H-pyrrolizin-7a(5H)-yl)methoxy)-9-methyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidine (Int-18E)

A vial with a stir bar was charged with ((2R,7aS)-2-fluorotetrahydro-1H-pyrrolizin-7a(5H)-yl)methanol (159 mg, 1.0 mmol), fitted with a septum, and placed under nitrogen. THF (3.9 mL) was added via syringe and the reaction was cooled to 0° C. Lithium diisopropylamide (1.1 mL, 1.1 mmol, 1M in THF) was added dropwise. A separate vial with stir bar was charged with 4-(tert-butoxy)-9-methyl-8-(6-methyl-1-(tetrahydro-2H-pyran-2-yl)-5-(trifluoromethyl)-1H-indazol-4-yl)-2-(methylsulfonyl)-9H-pyrido[4′,3′:4,5]pyrrolo[2,3-d]pyrimidine (Int-18D) (308 mg, 0.5 mmol), fitted with a septum, and placed under nitrogen. THF (2.5 mL) was added via syringe and the solution was cooled to −40° C. The lithium alkoxide solution (3.75 mL, 0.75 mmol, 0.2M in THF) was added dropwise via syringe to the sulfone solution. The reaction was warmed to room temperature and stirred for 1 h. The reaction was quenched with sat. aq. NH₄Cl (2 mL) and diluted with EtOAc (2 mL). The layers were separated and the aqueous phase was extracted with EtOAc (2×2 mL). The combined organics were dried over Na₂SO₄, filtered, and concentrated in vacuo. The crude material was purified via silica gel chromatography (0 to 50% (3:1 EtOAc:EtOH) in hexanes) to obtain 4-(tert-butoxy)-2-(((2R,7aS)-2-fluorotetrahydro-1H-pyrrolizin-7a(5H)-yl)methoxy)-9-methyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidine (Int-18E). MS (ESI) [M+H]⁺: m/z 696.

### Step F: 2-(((2R,7aS)-2-fluorotetrahydro-1H-pyrrolizin-7a(5H)-yl)methoxy)-9-methyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-ol (Int-18)

4-(tert-butoxy)-2-(((2R,7aS)-2-fluorotetrahydro-1H-pyrrolizin-7a(5H)-yl)methoxy)-9-methyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidine (Int-18E) (69 mg, 0.099 mmol) was added to a vial with a stir bar and dissolved in MeCN (763 μL), water (191 μL), and TFA (38.1 μL). The reaction was stirred at room temperature for 45 minutes. The reaction was quenched with sat. aq. NaHCO₃ (2 mL) and diluted with EtOAc (2 mL). The layers were separated and the aqueous phase was washed with EtOAc (2×2 mL). The combined organic layers were washed with brine (5 mL), dried over Na₂SO₄, filtered, and concentrated in vacuo to afford 2-(((2R,7aS)-2-fluorotetrahydro-1H-pyrrolizin-7a(5H)-yl)methoxy)-9-methyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-ol (Int-18), which was used without further purification. MS (ESI) [M+H]⁺: m/z 640.

### Intermediate 19: Tert-butyl (1R,5S)-3-(8-chloro-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate (Int-19)

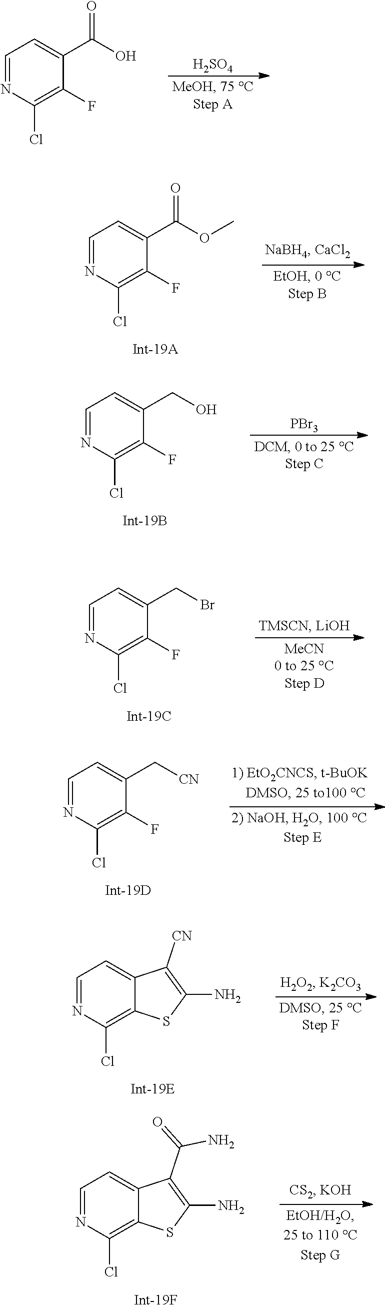

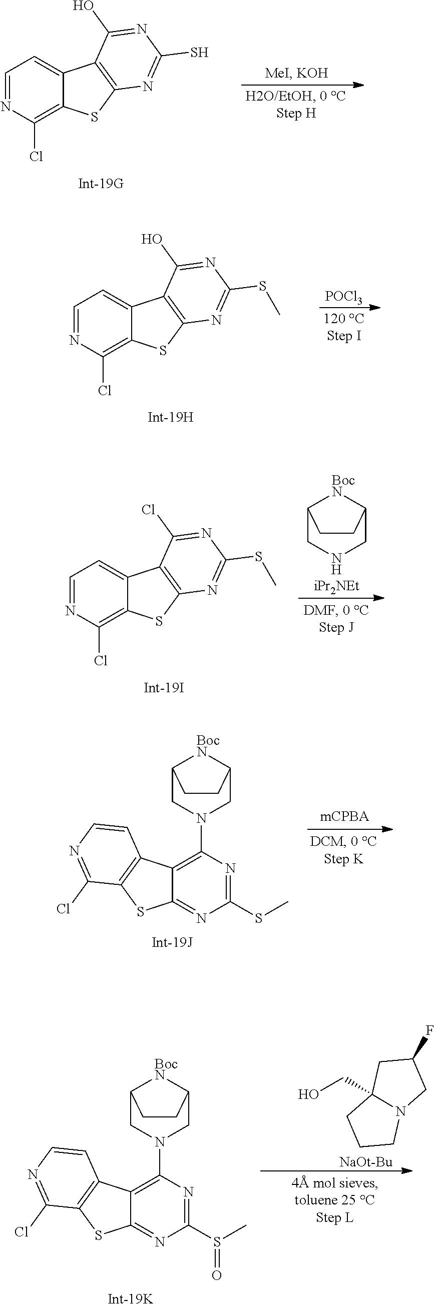

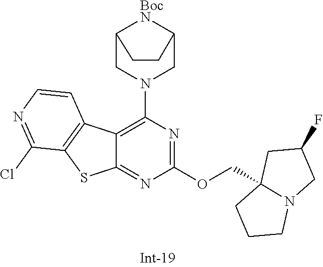 [SMILES: CC(C)(C)OC(=O)N1C2CCC1CN(c1nc(OC[C@@]34CCCN3C[C@H](F)C4)nc3sc4c(Cl)nccc4c13)C2]

### Step A: Methyl 2-chloro-3-fluoroisonicotinate (Int-19A)

Eight reactors were set up in parallel. To each reactor was charged MeOH (4.0 L) at 25° C., 2-chloro-3-fluoroisonicotinic acid (400 g, 2.3 mol), and conc. H₂SO₄ (45.0 g, 0.40 mol). The mixture was heated to 75° C. and stirred for 12 h. The contents of the eight reactors were combined and concentrated to remove the volatiles. The pH of the resulting residue was adjusted to ~7 using aq. Na₂CO₃ and the mixture was extracted with EtOAc (20 L×2). The combined organic layers were dried with Na₂SO₄ and concentrated under reduced pressure to give methyl 2-chloro-3-fluoroisonicotinate (Int-19A), which was used directly in the next step without further purification.

### Step B: (2-chloro-3-fluoropyridin-4-yl)methanol (Int-19B)

Twelve reactors were set up in parallel. To each reactor was charged EtOH (2.3 L), methyl 2-chloro-3-fluoroisonicotinate (Int-19A) (235 g, 1.2 mol), and CaCl₂ (206 g, 1.8 mol). The reactor was degassed and purged with N₂ three times and cooled to 0-10° C. NaBH₄ (93.0 g, 2.4 mol) was added at 0-10° C. and stirred for 1 hr. The mixture was warmed to 20-25° C. and stirred for 12 hr. The contents of the twelve reactors were combined and the mixture was slowly poured into ice water (5.0 V) under N₂ atmosphere. The mixture was filtered, and the filter cake was washed with EtOAc (5.0 V). The filtrate was concentrated under reduced pressure to give (2-chloro-3-fluoropyridin-4-yl)methanol (Int-19B). ¹H NMR (400 MHz, CDCl₃) δ 2.91 (s, 1H), 4.85 (s, 2H), 7.45-7.46 (t, J=4.0 Hz, 1H), 8.17-8.18 (d, J=4.0 Hz, 1H).

### Step C: 4-(bromomethyl)-2-chloro-3-fluoropyridine (Int-19C)

Twelve reactors were set up in parallel. To each reactor was charged DCM (2.0 L) and (2-chloro-3-fluoropyridin-4-yl)methanol (Int-19B) (200 g, 1.2 mol). PBr₃ (402 g, 1.5 mol) was charged into the reactor vessel at 0° C. The vessel was warmed to 25° C. and stirred for 12 hr. The contents of the twelve reactors were combined for workup and poured into 10% aq. NaHCO₃ (1.0 L) slowly. The reaction mixture was concentrated under reduced pressure. The resulting residue was diluted with DCM (20 L) and the organic phase was separate. The aqueous solution was extracted with DCM (2×20 L). The combined organic layers were dried with Na₂SO₄, filtered, and concentrated under reduce pressure to give 4-(bromomethyl)-2-chloro-3-fluoropyridine (Int-19C). MS (ESI) [M+H]⁺: m/z 224.

### Step D: 2-(2-chloro-3-fluoropyridin-4-yl)acetonitrile (Int-19D)

Eight reactors were set up in parallel. To each reactor was charged MeCN (2.3 L), 4-(bromomethyl)-2-chloro-3-fluoropyridine (Int-19C) (230 g, 1.0 mol), TMSCN (1.01 kg, 10 mol), and LiOH (51 g, 1.2 mol). The mixture was stirred at 0° C. for 4 hr and then warmed to room temperature and stirred for 8 hr. The contents of the eight reactors were combined and concentrated to remove the volatiles. The mixture was diluted with EtOAc (25 L) and H₂O (10 L) and the layers were separated. The aqueous phase was extracted with EtOAc (2×10 L) and the combined organic layers were dried with Na₂SO₄, filtered, and concentrated in vacuo to give 2-(2-chloro-3-fluoropyridin-4-yl)acetonitrile (Int-19D). MS (ESI) [M+H]⁺: m/z 171.

### Step E: 2-amino-7-chlorothieno[2,3-c]pyridine-3-carbonitrile (Int-19E)

Twelve reactors were set up in parallel. To each reactor was charged DMSO (1.7 L) and 2-(2-chloro-3-fluoropyridin-4-yl)acetonitrile (Int-19D) (170 g, 1.00 mol). The reactor was degassed and purged with N₂ three times. Potassium tert-butoxide (123 g, 1.1 mol) was added in batches at 25° C. over 30 min. The mixture was stirred at 25° C. for 30 min. After 30 min, ethoxycarbonyl isothiocyanate (143 g, 1.1 mol) was added at 25° C. The mixture was stirred at 25° C. for 30 min. The reactor was warmed to 100° C. and stirred at 100° C. for 1 hr. NaOH (1 L, 5.00 mol, 5 M in H₂O) was added and the mixture was stirred at 100° C. for 10 hr. The reaction was cooled to room temperature and the contents of the 12 reactors were combined for workup. The reaction was poured into ice water (10 V) and stirred at 0° C. for 30 min. The resulting solid was filtered, washed with H₂O (5.0 L), and oven-dried at 50° C. for 12 hr to obtain 2-amino-7-chlorothieno[2,3-c]pyridine-3-carbonitrile (Int-19E). MS (ESI) [M+H]⁺: m/z 210.

### Step F: 2-amino-7-chlorothieno[2,3-c]pyridine-3-carboxamide (Int-19F)

Six reactors were set up in parallel. To each reactor was charged DMSO (2.1 L), 2-amino-7-chlorothieno[2,3-c]pyridine-3-carbonitrile (Int-19E) (70.0 g, 0.3 mol), and K₂CO₃ (92.0 g, 0.60 mol). H₂O₂ (615 g, 5.4 mol, 30 wt %) was added slowly into the reactor in portions over 4 hr. The reaction mixture was stirred at 25° C. for 6 hr. The contents of the six reactors were combined, and the resulting mixture was poured into 10% aq. Na₂SO₃ at 15-25° C. The mixture was stirred at 0° C. for 0.5 hr. The reaction mixture was filtered, and the filter cake was washed with H₂O (5.0 L). The resulting solid was dried in an oven at 50° C. for 12 hr to give 2-amino-7-chlorothieno[2,3-c]pyridine-3-carboxamide (Int-19F). MS (ESI) [M+H]⁺: m/z 228. ¹H NMR (400 MHz, DMSO-d₆) δ 7.19-7.20 (d, J=4.0 Hz, 1H), 8.10-8.11 (d, J=4.0 Hz, 1H).

### Step G: 8-chloro-2-mercaptopyrido[4′,3′:4,5]thieno[2,3-d]pyrimidin-4-ol (Int-19G)

Five reactors were set up in parallel. To each reactor was charged EtOH (0.5 L) and KOH (52.0 g, 0.90 mol), and the mixture was stirred at 25° C. for 0.5 hr. CS₂ (70.0 g, 0.9 mol) was added at 25° C. and the mixture was stirred at 25° C. for 0.5 hr. H₂O (0.5 L) was added followed by 2-amino-7-chlorothieno[2,3-c]pyridine-3-carboxamide (Int-19F) (70.0 g, 0.3 mol). The mixture was heated to 110° C. and stirred for 12 hrs. The contents of the five reactors were combined. The volatiles were removed under reduced pressure and the pH was adjusted to ~3 with aq. HCl. The mixture was stirred at 0° C. for 1 hr, filtered, and washed with H₂O (5.0 L). The resulting solid was triturated with EtOH (10 V) at 25° C. for 8 hr and dried in an oven at 40° C. for 12 hr to give 8-chloro-2-mercaptopyrido[4′,3′:4,5]thieno[2,3-d]pyrimidin-4-ol (Int-19G). MS (ESI) [M+H]⁺: m/z 270.

### Step H: 8-chloro-2-(methylthio)pyrido[4′,3′:4,5]thieno[2,3-d]pyrimidin-4-ol (Int-19H)

Two reactors were set up in parallel. To each reactor was charged EtOH (1.0 L), H₂O (1.0 L), 8-chloro-2-mercaptopyrido[4′,3′:4,5]thieno[2,3-d]pyrimidin-4-ol (Int-19G) (135 g, 0.50 mol), and KOH (56.0 g, 1.0 mol). The mixture was stirred at 0° C. for 0.5 hr. MeI (85.0 g, 0.60 mol) was added at 0° C. and the mixture was stirred for 1.5 hr. The contents of the two reactors were combined. The volatiles were removed under reduced pressure and the pH was adjusted to ~3 with aq. HCl. The mixture was stirred at 0° C. for 1 hr, filtered, and washed with H₂O (5.0 L). The resulting solid was triturated with EtOH (10 V) at 25° C. for 8 hr and then dried in an oven at 40° C. for 12 hr to give 8-chloro-2-(methylthio)pyrido[4′,3′:4,5]thieno[2,3-d]pyrimidin-4-ol (Int-19H). MS (ESI) [M+H]⁺: m/z 284. ¹H NMR (400 MHz, DMSO-d₆) δ 2.62 (s, 3H), 8.16-8.17 (d, J=4.0 Hz, 1H), 8.44-8.45 (d, J=4.0 Hz, 1H), 13.4 (s, 1H).

### Step I: 4,8-dichloro-2-(methylthio)pyrido[4′3′:4,5]thieno[2,3-d]pyrimidine (Int-191)

Three reactors were set up in parallel. To each reactor was charged POCl₃ (2.02 kg, 13 mol) and 8-chloro-2-(methylthio)pyrido[4′,3′:4,5]thieno[2,3-d]pyrimidin-4-ol (Int-19H) (55.0 g, 0.20 mol). The reaction mixture was stirred at 120° C. for 5 hrs. The contents of the three reactors were combined. The resulting mixture was concentrated under reduced pressure and the residue was triturated with EtOH (10 V) at 25° C. for 8 hr. The resulting solid was dried in an oven at 40° C. for 12 hr to give 4,8-dichloro-2-(methylthio)pyrido[4′,3′:4,5]thieno[2,3-d]pyrimidine (Int-19I). MS (ESI) [M+H]⁺: m/z 302. ¹H NMR (400 MHz, DMSO-d₆) δ 2.67 (s, 3H), 8.48-8.49 (d, J=4.0 Hz, 1H), 8.65-8.66 (d, J=4.0 Hz, 1H).

### Step J: Tert-butyl (1R,5S)-3-(8-chloro-2-(methylthio)pyrido[4′,3′:4,5]thieno[2,3-d]pyrimidin-4-yl)-3,8-diazabicyclo[3.2.1]octane-8-carboxylate (Int-19J)

To a stirred mixture of 4,8-dichloro-2-(methylthio)pyrido[4′,3′:4,5]thieno[2,3-d]pyrimidine (Int-19I) (170 mg, 0.56 mmol) and N,N-diisopropylethylamine (300 μL, 1.69 mmol) in DMF (5 mL) was added tert-butyl (1R,5S)-3,8-diazabicyclo[3.2.1]octane-8-carboxylate (119 mg, 0.56 mmol) at 0° C. The mixture was warmed to room temperature and stirred for 1 h. The mixture was diluted with EtOAc and water and the layers were separated. The organic phase was washed with water and brine, dried over Na₂SO₄, filtered, and concentrated in vacuo. The residue was purified via silica gel chromatography (30 to 50% EtOAc in hexane) to give tert-butyl (1R,5S)-3-(8-chloro-2-(methylthio)pyrido[4′,3′:4,5]thieno[2,3-d]pyrimidin-4-yl)-3,8-diazabicyclo[3.2.1]octane-8-carboxylate (Int-19J). MS (ESI) [M+H]⁺: m/z 478. ¹H NMR (400 MHz, CDCl₃) δ=8.43 (d, J=5.5 Hz, 1H), 7.47 (d, J=5.5 Hz, 1H), 4.48-4.21 (m, 3H), 4.18-3.99 (m, 1H), 3.84-3.38 (m, 2H), 2.65 (s, 3H), 1.96-1.84 (m, 2H), 1.82-1.63 (m, 2H), 1.55 (s, 9H).

### Step K: Tert-butyl (1R,5S)-3-(8-chloro-2-(methylsulfinyl)pyrido[4′,3′:4,5]thieno[2,3-d]pyrimidin-4-yl)-3,8-diazabicyclo[3.2.1]octane-8-carboxylate (Int-19K)

To a stirring solution of tert-butyl (1R,5S)-3-(8-chloro-2-(methylthio)pyrido[4′,3′:4,5]thieno[2,3-d]pyrimidin-4-yl)-3,8-diazabicyclo[3.2.1]octane-8-carboxylate (Int-19J) (200 mg, 2.52 mmol) in DCM (2.5 mL) was added m-chloroperoxybenzoic acid (580 mg, 2.52 mmol, 75 wt %) at 0° C. The mixture was quenched with sat. aq. NaHCO₃ and the mixture was extracted with CHCl₃. The combined organic layers were washed with brine, dried over Na₂SO₄, filtered, and concentrated. The crude residue was purified via silica gel chromatography (60 to 100% EtOAc in hexane) to give tert-butyl (1R,5S)-3-(8-chloro-2-(methylsulfinyl)pyrido[4′,3′:4,5]thieno[2,3-d]pyrimidin-4-yl)-3,8-diazabicyclo[3.2.1]octane-8-carboxylate (Int-19K). MS (ESI) [M+H]⁺: m/z 494. ¹H NMR (400 MHz, CDCl₃) δ=8.52 (d, J=5.6 Hz, 1H), 7.58 (d, J=5.5 Hz, 1H), 4.62-4.28 (m, 3H), 4.23-4.02 (m, 1H), 3.93-3.47 (m, 2H), 3.01 (s, 3H), 1.96-1.87 (m, 2H), 1.83-1.61 (m, 2H), 1.56 (s, 9H)

### Step L: Tert-butyl (1R,5S)-3-(8-chloro-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate (Int-19)

To a stirring mixture of tert-butyl (1R,5S)-3-(8-chloro-2-(methylsulfinyl)pyrido[4′,3′:4,5]thieno[2,3-d]pyrimidin-4-yl)-3,8-diazabicyclo[3.2.1]octane-8-carboxylate (Int-19K) (30 mg, 0.060 mmol), ((2R,7aS)-2-fluorotetrahydro-1H-pyrrolizin-7a(5H)-yl)methanol (12 mg, 0.073 mmol), 4 Å molecular sieves, and toluene (2 mL) was added sodium tert-butoxide (20 mg, 0.121 mmol) at 25° C., and the mixture was stirred for 1.5 hr. The mixture was diluted with water and EtOAc. The organic layer was separated, dried over Na₂SO₄, filtered, and concentrated in vacuo. The residue was purified via flash NH-silica gel chromatography (25 to 60% EtOAc in hexane) to give tert-butyl (1R,5S)-3-(8-chloro-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate (Int-19). MS (ESI) [M+H]⁺: m/z 589. ¹H NMR (400 MHz, CDCl₃) δ=8.41 (d, J=5.5 Hz, 1H), 7.44 (d, J=5.5 Hz, 1H), 5.43-5.17 (m, 1H), 4.46-3.99 (m, 6H), 3.85-3.41 (m, 2H), 3.37-3.23 (m, 2H), 3.23-3.11 (m, 1H), 3.07-2.95 (m, 1H), 2.40-2.24 (m, 2H), 2.20-2.10 (m, 2H), 2.06-1.96 (m, 2H), 1.93-1.83 (m, 4H), 1.55 (s, 9H).

### Intermediate 20: Tert-butyl (1R,5S)-3-(2-(((R)-1-((benzyloxy)methyl)-2,2-difluorocyclopropyl)methoxy)-8-chloropyrido[4′,3′:4,5]thieno[2,3-d]pyrimidin-4-yl)-3,8-diazabicyclo[3.2.1]octane-8-carboxylate (Int-20)

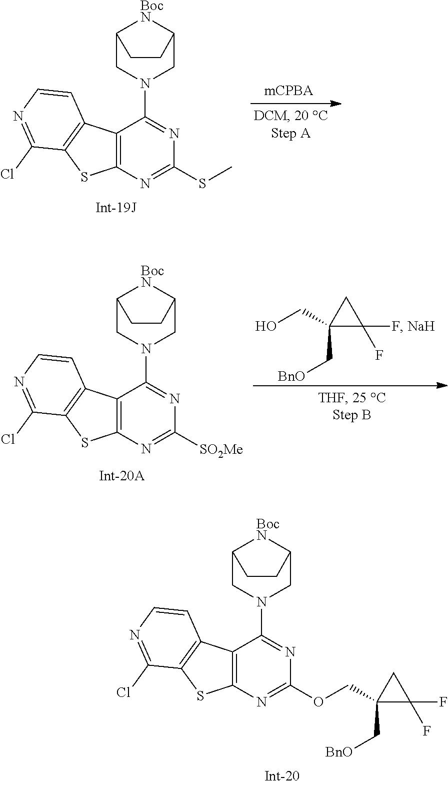

### Step A: Tert-butyl (1R,5S)-3-(8-chloro-2-(methylsulfonyl)pyrido[4′,3′:4,5]thieno[2,3-d]pyrimidin-4-yl)-3,8-diazabicyclo[3.2.1]octane-8-carboxylate (Int-20A)

To a solution of tert-butyl (1R,5S)-3-(8-chloro-2-(methylthio)pyrido[4′,3′:4,5]thieno[2,3-d]pyrimidin-4-yl)-3,8-diazabicyclo[3.2.1]octane-8-carboxylate (Int-19J) (10 g, 20.9 mmol) in DCM (100 mL) was added m-chloroperoxybenzoic acid (12.7 g, 62.8 mmol, 85 wt %) at room temperature, and the mixture was stirred for 20 minutes. The reaction was filtered and the filter cake was washed with DCM (30 mL). The filtrate was quenched with sat. aq. Na₂SO₃ (30 mL), and NaHCO₃ (50 mL). The reaction mixture was stirred for 30 min. The mixture was extracted with DCM (3×50 mL), and the combined organic layer was washed with sat. aq. NaHCO₃ (20 mL), brine (30 mL), dried over Na₂SO₄, filtered, and concentrated under reduced pressure to afford tert-butyl (1R,5S)-3-(8-chloro-2-(methylsulfonyl)pyrido[4′,3′:4,5]thieno[2,3-d]pyrimidin-4-yl)-3,8-diazabicyclo[3.2.1]octane-8-carboxylate (Int-20A), which was used directly in the next step without further purification. MS (ESI) [M+H]⁺: m/z 510.

### Step B: Tert-butyl (1R,5S)-3-(2-(((R)-1-((benzyloxy)methyl)-2,2-difluorocyclopropyl)methoxy)-8-chloropyrido[4′,3′:4,5]thieno[2,3-d]pyrimidin-4-yl)-3,8-diazabicyclo[3.2.1]octane-8-carboxylate (Int-20)

To a solution of (S)-(1-((benzyloxy)methyl)-2,2-difluorocyclopropyl)methanol (3.36 g, 14.71 mmol) in THF (20 mL) was added NaH (0.784 g, 19.6 mmol, 60 wt % in mineral oil) at 25° C. The mixture was stirred at room temperature for 10 min. A solution of tert-butyl (1R,5S)-3-(8-chloro-2-(methylsulfonyl)pyrido[4′,3′:4,5]thieno[2,3-d]pyrimidin-4-yl)-3,8-diazabicyclo[3.2.1]octane-8-carboxylate (Int-20A) (5.0 g, 9.80 mmol) in THF (20 mL) was added to the reaction solution and the mixture was stirred at room temperature for 30 min. The mixture was diluted with water (40 mL), extracted with DCM (3×40 mL), dried over Na₂SO₄, filtered, and the solvent was evaporated under reduced pressure. The residue was purified by flash silica gel chromatography (0 to 30% EtOAc in petroleum ether) to give tert-butyl (1R,5S)-3-(2-(((R)-1-((benzyloxy)methyl)-2,2-difluorocyclopropyl)methoxy)-8-chloropyrido[4′,3′:4,5]thieno[2,3-d]pyrimidin-4-yl)-3,8-diazabicyclo[3.2.1]octane-8-carboxylate (Int-20). MS (ESI) [M+H]⁺: m/z 658.

The compounds in the table below were synthesized using a similar procedure as described in the synthesis of Int-20 by making the appropriate substitutions for starting material, intermediates, and/or reagents. Appropriate substitutions are available commercially, synthesized as described in the literature, synthesized using methods available to those skilled in the art, or synthesized as described herein.

| | | | | [M + |
|---|---|---|---|---|
| | | | | H]⁺ |
| Int. | Starting material | Structure | Compound Name | Found |
| Int- 21 | 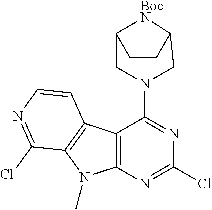 [SMILES: Cn1c2nc(Cl)nc(N3CC4CCC(C3)N4C(=O)OC(C)(C)C)c2c2ccnc(Cl)c21] Int-17G | 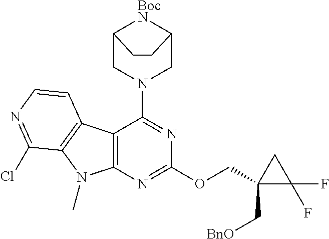 [SMILES: Cn1c2nc(OC[C@]3(COCc4ccccc4)CC3(F)F)nc(N3CC4CCC(C3)N4C(=O)OC(C)(C)C)c2c2ccnc(Cl)c21] | tert-butyl (1R,5S)-3-(2-(((R)- (1-(benzyloxy)methyl)-2,2- difluorocyclopropyl)meth- oxy)-8-chloro-9-meth- yl-9H- pyrido[4′,3′:4,5]pyrrolo[2,3- d]pyrimidin-4- yl)-3,8- diazabicyclo[3.2.1]octane-8- carboxylate | 655 |

### Intermediate 43: tert-butyl (1R,5S)-3-(2-(methylsulfinyl)-8-(5-((triisopropylsilyl)ethynyl)-1-((2-(trimethylsilyl)ethoxy)methyl)-1H-thieno[3,2-f]indazol-4-yl)pyrido[4′,3′:4,5]thieno[2,3-d]pyrimidin-4-yl)-3,8-diazabicyclo[3.2.1]octane-8-carboxylate (Int-43)

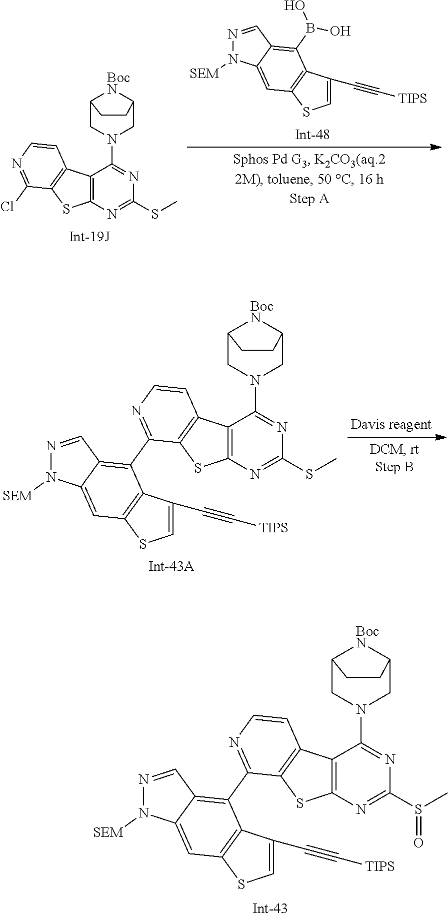

### Step A: tert-butyl (1R,5S)-3-(2-(methylthio)-8-(5-((triisopropylsilyl)ethynyl)-1-((2-(trimethylsilyl)ethoxy)methyl)-1H-thieno[3,2-f]indazol-4-yl)pyrido[4′,3′:4,5]thieno[2,3-d]pyrimidin-4-yl)-3,8-diazabicyclo[3.2.1]octane-8-carboxylate (Int-43A)

To a solution of tert-butyl (1R,5S)-3-(8-chloro-2-(methylthio)pyrido[4′,3′:4,5]thieno[2,3-d]pyrimidin-4-yl)-3,8-diazabicyclo[3.2.1]octane-8-carboxylate (Int-19J) (160 mg, 0.335 mmol) in toluene (2.5 mL) was added (5-((triisopropylsilyl)ethynyl)-1-((2-(trimethylsilyl)ethoxy)methyl)-1H-thieno[3,2-J]indazol-4-yl)boronic acid (Int-48) (212 mg, 0.402 mmol), potassium carbonate (0.502 mL, 1.00 mmol, 2 M in water) and SPhos Pd G3 (131 mg, 0.167 mmol) at 25° C. The mixture was stirred at 50° C. for 16 h under N₂ atmosphere. The mixture was extracted with EtOAc (10 mL×3). The organic layer was dried over Na₂SO₄, filtered and the filtrate was evaporated under reduced pressure. The residue was purified by preparative TLC (petroleum ether:ethyl acetate=2:1) to give tert-butyl (1R,5S)-3-(2-(methylthio)-8-(5-((triisopropylsilyl)ethynyl)-1-((2-(trimethylsilyl)ethoxy)methyl)-1H-thieno[3,2-J]indazol-4-yl)pyrido[4′,3′:4,5]thieno[2,3-d]pyrimidin-4-yl)-3,8-diazabicyclo[3.2.1]octane-8-carboxylate (Int-43A). MS (ESI) [M+H]⁺: m/z 926.

### Step B: tert-butyl (1R,5S)-3-(2-(methylsulfinyl)-8-(5-((triisopropylsilyl)ethynyl)-1-((2-(trimethylsilyl)ethoxy)methyl)-1H-thieno[3,2-f]indazol-4-yl)pyrido[4′,3′:4,5]thieno[2,3-d]pyrimidin-4-yl)-3,8-diazabicyclo[3.2.1]octane-8-carboxylate (Int-43)

To a solution of tert-butyl (1R,5S)-3-(2-(methylthio)-8-(5-((triisopropylsilyl)ethynyl)-1-((2-(trimethylsilyl)ethoxy)methyl)-1H-thieno[3,2-J]indazol-4-yl)pyrido[4′,3′:4,5]thieno[2,3-d]pyrimidin-4-yl)-3,8-diazabicyclo[3.2.1]octane-8-carboxylate (Int-43A) (641 mg, 0.692 mmol) in DCM (7 mL) was added 3-phenyl-2-(phenylsulfonyl)-1,2-oxaziridine (651 mg, 2.491 mmol) at 20° C. under N₂ atmosphere. The mixture was stirred at 20° C. for 2 h. The mixture was evaporated under reduced pressure. The product was purified by flash silica gel chromatography (0~30% EtOAc/Pet. ether) to give tert-butyl (1R,5S)-3-(2-(methylsulfinyl)-8-(5-((triisopropylsilyl)ethynyl)-1-((2-(trimethylsilyl)ethoxy)methyl)-1H-thieno[3,2-f]indazol-4-yl)pyrido[4′,3′:4,5]thieno[2,3-d]pyrimidin-4-yl)-3,8-diazabicyclo[3.2.1]octane-8-carboxylate (Int-43). MS (ESI) [M+H]⁺: m/z 942.

### Intermediate 22: 2-(((2R,7aS)-2-fluorotetrahydro-1H-pyrrolizin-7a(5H)-yl)methoxy)-8-(6-methyl-5-(trifluoromethyl)-1H-indazol-4-yl)pyrido[4′,3′:4,5]thieno[2,3-d]pyrimidin-4-ol (Int-22)

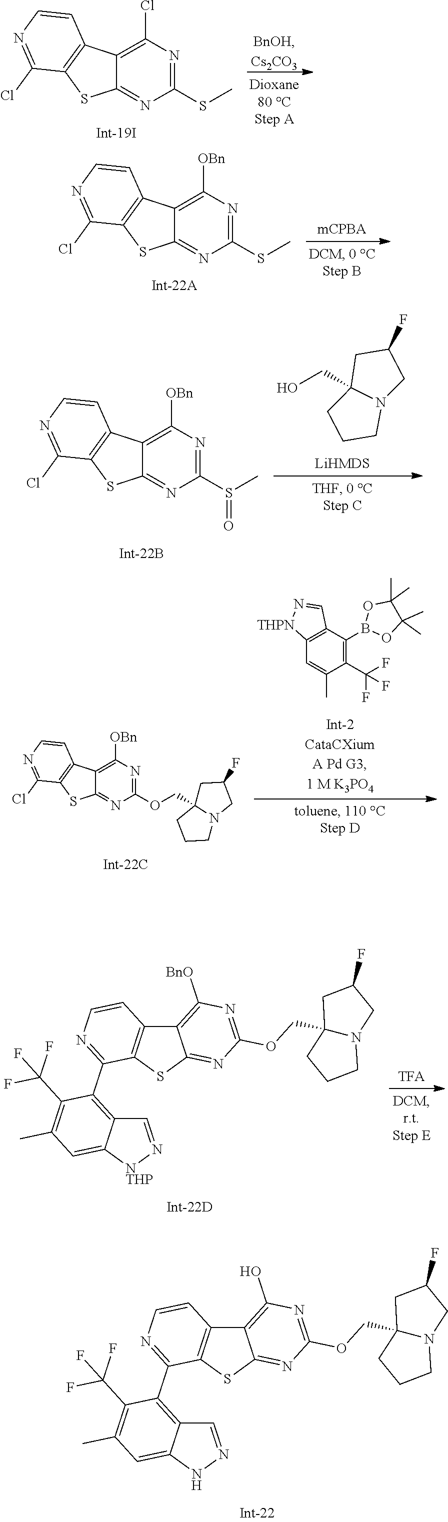

### Step A: 4-(benzyloxy)-8-chloro-2-(methylthio)pyrido[4′,3′:4,5]thieno[2,3-d]pyrimidine (Int-22A)

Benzyl alcohol (1.0 mL, 9.9 mmol) and Cs₂CO₃ (3.3 g, 10 mmol) were added to a solution of 4,8-dichloro-2-(methylthio)pyrido[4′,3′:4,5]thieno[2,3-d]pyrimidine (Int-191) (1 g, 3.3 mmol) in 1,4-dioxane (17 mL). The mixture was heated to 80° C. and stirred for 5 h. After 5 h, the reaction was cooled to room temperature. Water was added and the resulting solid was collected by filtration. The solid was heated to 50° C. under vacuum to dry, thereby obtaining 4-(benzyloxy)-8-chloro-2-(methylthio)pyrido[4′,3′:4,5]thieno[2,3-d]pyrimidine (Int-22A). MS (ESI): m/z (M+H)+374.

### Step B: 4-(benzyloxy)-8-chloro-2-(methylsulfinyl)pyrido[4′,3′:4,5]thieno[2,3-d]pyrimidine (Int-22B)

mCPBA (610 mg, 2.6 mmol) was added to a solution of 4-(benzyloxy)-8-chloro-2-(methylthio)pyrido[4′,3′:4,5]thieno[2,3-d]pyrimidine (Int-22A) (900 mg, 2.5 mmol) in DCM (60 mL) at 0° C. The mixture was stirred at 0° C. for 30 min. EtOAc, water, and sat. aq. NaHCO₃ were added and the layers were separated. The organic phase was washed with sat. aq. NaHCO₃ and brine, dried over sodium sulfate, filtered, and concentrated to give crude 4-(benzyloxy)-8-chloro-2-(methylsulfinyl)pyrido[4′,3′:4,5]thieno[2,3-d]pyrimidine (Int-22B) which was used directly in the next step without further purification. MS (ESI): m/z (M+H)+390.

### Step C: 4-(benzyloxy)-8-chloro-2-(((2R,7aS)-2-fluorotetrahydro-1H-pyrrolizin-7a(5H)-yl)methoxy)pyrido[4′,3′:4,5]thieno[2,3-d]pyrimidine (Int-22C)

LiHMDS (4.8 mL, 4.8 mmol, 1M in THF) was added to a solution of ((2R,7aS)-2-fluorotetrahydro-1H-pyrrolizin-7a(5H)-yl)methanol (770 mg, 4.8 mmol) in THF (24 mL) at 0° C. The mixture was stirred at 0° C. for 20 min. The solution of crude 4-(benzyloxy)-8-chloro-2-(methylsulfinyl)pyrido[4′,3′:4,5]thieno[2,3-d]pyrimidine (Int-22B) in THF (24 mL) was added dropwise at 0° C. and the reaction was stirred at 0° C. for 15 min. Water, EtOAc, and sat. aq. NaHCO₃ were added at 0° C. and the organic phase was washed with sat. aq. NaHCO₃ and brine, dried over sodium sulfate, filtered, and concentrated. The crude mixture was purified via silica gel chromatography (0 to 3% MeOH in chloroform) to give 4-(benzyloxy)-8-chloro-2-(((2R,7aS)-2-fluorotetrahydro-1H-pyrrolizin-7a(5H)-yl)methoxy)pyrido[4′,3′:4,5]thieno[2,3-d]pyrimidine (Int-22C). MS (ESI): m/z (M+H)+485.

### Step D: 4-(benzyloxy)-2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)pyrido[4′,3′:4,5]thieno[2,3-d]pyrimidine (Int-22D)

To a mixture of 4-(benzyloxy)-8-chloro-2-(((2R,7aS)-2-fluorotetrahydro-1H-pyrrolizin-7a(5H)-yl)methoxy)pyrido[4′,3′:4,5]thieno[2,3-d]pyrimidine (Int-22C) (220 mg, 0.45 mmol), 6-methyl-1-(tetrahydro-2H-pyran-2-yl)-4-(4,4,5,5-tetramethyl-1,3,2-dioxaborolan-2-yl)-5-(trifluoromethyl)-1H-indazole (Int-2) (320 mg, 0.77 mmol), and cataCXium A Pd G3 (170 mg, 0.23 mmol) in toluene (9.1 mL) was added K₃PO₄ (2.3 mL, 2.3 mmol, 1M in H₂O). The mixture was stirred at 110° C. for 6 hr. After 6 hr, the reaction was cooled to room temperature. Water and EtOAc were added and the layers were separated. The organic phase was washed with brine, dried over sodium sulfate, filtered, and concentrated. The crude residue was purified by silica gel chromatography (0 to 4% MeOH in chloroform) to give 4-(benzyloxy)-2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)pyrido[4′,3′:4,5]thieno[2,3-d]pyrimidine (Int-22D). MS (ESI): m/z (M+H)+733.

### Step E: 2-(((2R,7aS)-2-fluorotetrahydro-1H-pyrrolizin-7a(5H)-yl)methoxy)-8-(6-methyl-5-(trifluoromethyl)-1H-indazol-4-yl)pyrido[4′,3′:4,5]thieno[2,3-d]pyrimidin-4-ol (Int-22)

To a mixture of 4-(benzyloxy)-2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)pyrido[4′,3′:4,5]thieno[2,3-d]pyrimidine (Int-22D) (20 mg, 0.027 mmol) in DCM (0.55 mL) was added TFA (0.30 mL, 4.1 mmol). The mixture was stirred at room temperature for 2.5 hr. After 2.5 hr, the reaction was concentrated to give crude 2-(((2R,7aS)-2-fluorotetrahydro-1H-pyrrolizin-7a(5H)-yl)methoxy)-8-(6-methyl-5-(trifluoromethyl)-1H-indazol-4-yl)pyrido[4′,3′:4,5]thieno[2,3-d]pyrimidin-4-ol (Int-22), which was used without further purification. MS (ESI): m/z (M+H)+559.

### Intermediate 44: 4-(benzyloxy)-8-chloro-2-(methylthio)-9H-pyrido[4′,3′:3,4]cyclopenta[1,2-d]pyrimidin-9-one (Int-44)

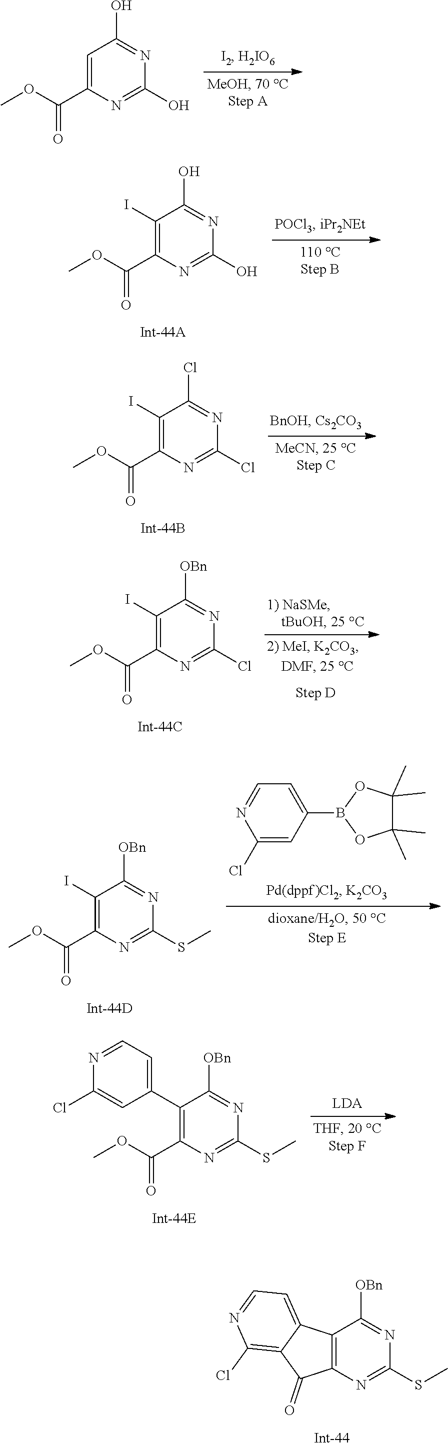

### Step A: Methyl 2,6-dihydroxy-5-iodopyrimidine-4-carboxylate (Int-44A)

Periodic acid (9.04 g, 39.7 mmol) and iodine (23.5 g, 93 mmol) were added to a solution of methyl 2,6-dihydroxypyrimidine-4-carboxylate (37.5 g, 220 mmol) in MeOH (350 mL) at 25° C. The mixture was heated to 70° C. and stirred for 16 h. The mixture was concentrated in vacuo, and the residue was diluted with H₂O (200 mL). The resulting mixture was filtered, and the filter cake was washed with H₂O (2×50 mL). The filter cake was dried via azeotropic distillation using toluene to give methyl 2,6-dihydroxy-5-iodopyrimidine-4-carboxylate (Int-44A). MS (ESI) [M+H]⁺: m/z 297.

### Step B: Methyl 2,6-dichloro-5-iodopyrimidine-4-carboxylate (Int-44B)

DIPEA (73.0 mL, 418 mmol) was added to a solution of methyl 2,6-dihydroxy-5-iodopyrimidine-4-carboxylate (Int-44A) (55 g, 186 mmol) in phosphoryl chloride (550 mL) at 25° C. The mixture was heated to 110° C. for 6 h. The mixture was cooled to rt and was concentrated by distillation followed by azeotropic distillation using toluene (2×200 mL). The residue was purified by flash silica gel chromatography (petroleum ether/EtOAc=10/1) to give methyl 2,6-dichloro-5-iodopyrimidine-4-carboxylate (Int-44B). ¹H NMR (400 MHz, CDCl₃) δ 4.05 (s, 3H).

### Step C: Methyl 6-(benzyloxy)-2-chloro-5-iodopyrimidine-4-carboxylate (Int-44C)

Cs₂CO₃ (63.7 g, 196 mmol) was added to a solution of methyl 2,6-dichloro-5-iodopyrimidine-4-carboxylate (Int-44B) (37.2 g, 112 mmol) and benzyl alcohol (10.88 g, 101 mmol) in MeCN (375 mL) at 25° C. The mixture was stirred at 25° C. for 12 h. The mixture was diluted with EtOAc (375 mL), filtered, and the filtrate was concentrated in vacuo. The residue was purified by flash silica gel chromatography (petroleum ether/EtOAc=20/1) to give methyl 6-(benzyloxy)-2-chloro-5-iodopyrimidine-4-carboxylate (Int-44C). MS (ESI) [M+H]⁺: m/z 405.

### Step D: Methyl 6-(benzyloxy)-5-iodo-2-(methylthio)pyrimidine-4-carboxylate (Int-44D)

Sodium methanethiolate (8.45 g, 121 mmol) was added to a solution of methyl 6-(benzyloxy)-2-chloro-5-iodopyrimidine-4-carboxylate (Int-44C) (28.7 g, 70.9 mmol) in tert-butanol (450 mL) at 25° C., and the mixture was stirred for 16 h under N₂ atmosphere. The mixture was quenched with water (100 mL) and extracted with EtOAc (3×500 mL). The organic layer was dried over Na₂SO₄, filtered, and concentrated in vacuo to give 6-(benzyloxy)-5-iodo-2-(methylthio)pyrimidine-4-carboxylic acid, which was used in the next step without further purification. MS (ESI) [M+H]⁺: m/z 403.

K₂CO₃ (49.0 g, 354 mmol) and iodomethane (17.7 mL, 283 mmol) was added to a solution of 6-(benzyloxy)-5-iodo-2-(methylthio)pyrimidine-4-carboxylic acid (28.5 g, 70.9 mmol) in DMF (300 mL) at 25° C., and the mixture was stirred for 16 h under N₂ atmosphere. The mixture was diluted with H₂O (100 mL) and extracted with EtOAc (3×300 mL). The combined organic layer was washed with brine (3×40 mL), dried over Na₂SO₄, filtered, and concentrated in vacuo. The residue was purified by flash silica gel chromatography (0 to 18% petroleum ether in THF) to afford methyl 6-(benzyloxy)-5-iodo-2-(methylthio)pyrimidine-4-carboxylate (Int-44D). MS (ESI) [M+H]⁺: m/z 417.

### Step E: Methyl-6-(benzyloxy)-5-(2-chloropyridin-4-yl)-2-(methylthio)pyrimidine-4-carboxylate (Int-44E)

2-chloro-4-(4,4,5,5-tetramethyl-1,3,2-dioxaborolan-2-yl)pyridine (6.28 g, 26.2 mmol), K₂CO₃ (7.77 g, 56.2 mmol) and Pd(dppf)Cl₂ (2.74 g, 3.75 mmol) were added to a solution of methyl 6-(benzyloxy)-5-iodo-2-(methylthio)pyrimidine-4-carboxylate (Int-44D) (7.8 g, 18.7 mmol) in dioxane (100 mL) under N₂ atmosphere, and the mixture was stirred at 50° C. for 4 h. The mixture was diluted with H₂O (40 mL), and the resulting mixture was extracted with EtOAc (3×200 mL). The combined organic phase was washed with brine, dried over anhydrous Na₂SO₄, filtered, and concentrated in vacuo. The residue was purified by flash silica gel chromatography (petroleum ether/EtOAc=5/1) to give methyl 6-(benzyloxy)-5-(2-chloropyridin-4-yl)-2-(methylthio)pyrimidine-4-carboxylate (Int-44E).

MS (ESI) [M+H]⁺: m/z 402.

### Step F: 4-(benzyloxy)-8-chloro-2-(methylthio)-9H-pyrido[4′,3′:3,4]cyclopenta[1,2-d]pyrimidin-9-one (Int-44)

The reaction was set up using flow chemistry. Methyl 6-(benzyloxy)-5-(2-chloropyridin-4-yl)-2-(methylthio)pyrimidine-4-carboxylate (Int-44E) (5.6 g, 13.9 mmol) was dissolved in THF (120 mL) and charged into Reservoir 1 at 20° C. Lithium diisopropylamide (20.90 mL, 41.8 mmol, 2 M in THF) and THF (120 mL) was charged into Reservoir 2 at 20° C. The flow rate for Reservoir 1 and Reservoir 2 was adjusted to 12.0 mL/min with the total reaction residence time being 15 s. The mixture was quenched with aq. NH₄Cl, the organic phase was collected, and the pumps and tubing were washed with THF. The mixture was diluted with additional sat. aq. NH₄Cl (80 mL), and the mixture was extracted with EtOAc (200 mL×3). The combined organic layers were washed with brine, dried over anhydrous Na₂SO₄, filtered, and concentrated in vacuo. The residue was purified by flash silica gel chromatography (petroleum ether/THF=5/1) to give 4-(benzyloxy)-8-chloro-2-(methylthio)-9H-pyrido[4′,3′:3,4]cyclopenta[1,2-d]pyrimidin-9-one (Int-44). MS (ESI) [M+H]⁺: m/z 370.

### EXAMPLE SYNTHESES

### Example 1: 4-((1R,5S)-3,8-diazabicyclo[3.2.1]octan-3-yl)-2-(((2R,7aS)-2-fluorotetrahydro-1H-pyrrolizin-7a(5H)-yl)methoxy)-9-methyl-8-(6-methyl-5-(trifluoromethyl)-1H-indazol-4-yl)-9H-pyrido[4′,3′:4,5]pyrrolo[2,3-d]pyrimidine (Ex-1)

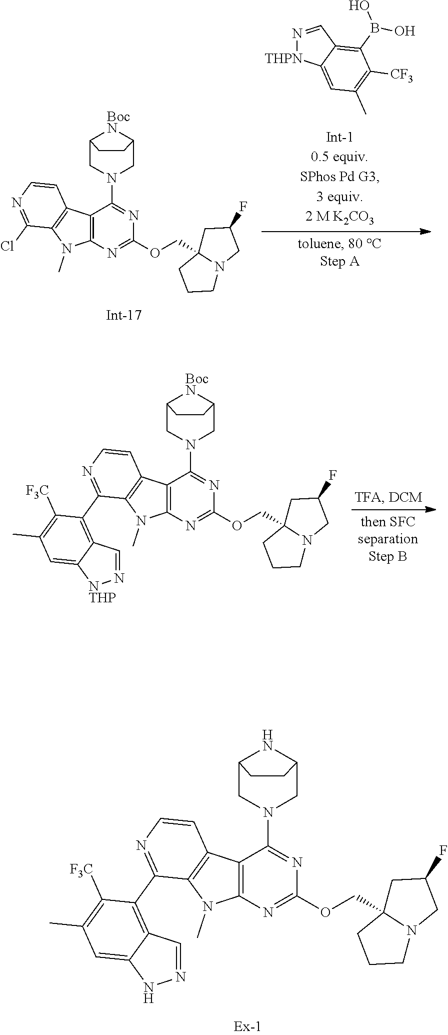

### Step A: Tert-butyl (1R,5S)-3-(2-(((2R,7aS)-2-fluorotetrahydro-1H-pyrrolizin-7a(5H)-yl)methoxy)-9-methyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-yl)-3,8-diazabicyclo[3.2.1]octane-8-carboxylate

To a solution of tert-butyl (1R,5S)-3-(8-chloro-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate (Int-17) (200 mg, 0.341 mmol) in toluene (0.5 mL) was added (6-methyl-1-(tetrahydro-2H-pyran-2-yl)-5-(trifluoromethyl)-1H-indazol-4-yl)boronic acid (Int-1) (224 mg, 0.682 mmol), K₂CO₃ (0.682 mL, 1.37 mmol, 2 M in H₂O) and SPhos Pd G3 (200 mg, 0.256 mmol) at 20° C. in a glovebox, and the mixture was stirred at 80° C. for 2 h under N₂ atmosphere. The reaction mixture was removed from the glovebox, diluted with EtOAc (30 mL), and washed with H₂O (2×3 mL) and brine (2×3 mL). The organic phase was dried over anhydrous Na₂SO₄, filtered, and concentrated in vacuo. The residue was purified by flash silica gel chromatography (petroleum ether/THF=1/1) to give tert-butyl (1R,5S)-3-(2-(((2R,7aS)-2-fluorotetrahydro-1H-pyrrolizin-7a(5H)-yl)methoxy)-9-methyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-yl)-3,8-diazabicyclo[3.2.1]octane-8-carboxylate. MS (ESI) [M+H]⁺: m/z 834.

### Step B: 4-((1R,5S)-3,8-diazabicyclo[3.2.1]octan-3-yl)-2-(((2R,7aS)-2-fluorotetrahydro-1H-pyrrolizin-7a(5H)-yl)methoxy)-9-methyl-8-(6-methyl-5-(trifluoromethyl)-1H-indazol-4-yl)-9H-pyrido[4′,3′:4,5]pyrrolo[2,3-d]pyrimidine (Ex-1)

To a solution of tert-butyl (1R,5S)-3-(2-(((2R,7aS)-2-fluorotetrahydro-1H-pyrrolizin-7a(5H)-yl)methoxy)-9-methyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-yl)-3,8-diazabicyclo[3.2.1]octane-8-carboxylate (330 mg, 0.301 mmol) in DCM (3 mL) was added TFA (1 mL) at 20° C., and the mixture was stirred at 20° C. for 1 h. The reaction mixture was concentrated in vacuo. The residue was diluted with 10% MeOH in DCM (20 mL) and basified with sat. aq. NaHCO₃. The organic phase was separated and dried over anhydrous Na₂SO₄, filtered, and concentrated in vacuo. The crude reaction mixture was purified via preparative-scale SFC (Arginine, 40% MeOH w/NH₄OH modifier). The atropisomers were separated via preparative-scale chiral SFC (Column B, 45% MeOH with 0.1% NH₄OH modifier) to give 4-((1R,5S)-3,8-diazabicyclo[3.2.1]octan-3-yl)-2-(((2R,7aS)-2-fluorotetrahydro-1H-pyrrolizin-7a(5H)-yl)methoxy)-9-methyl-8-(6-methyl-5-(trifluoromethyl)-1H-indazol-4-yl)-9H-pyrido[4′,3′:4,5]pyrrolo[2,3-d]pyrimidine (Ex-1, first eluting isomer from SFC). MS (ESI) [M+H]⁺: m/z 650. ¹H NMR (600 MHz, DMSO-d₆) δ 13.54 (s, 1H), 8.40 (d, J=5.4 Hz, 1H), 7.75 (d, J=5.3 Hz, 2H), 7.35 (s, 1H), 5.26 (d, J=53.5 Hz, 1H), 4.36 (dd, J=49.6, 11.7 Hz, 2H), 4.03 (dd, J=64.2, 10.2 Hz, 2H), 3.57 (d, J=12.2 Hz, 2H), 3.49 (d, J=11.7 Hz, 1H), 3.07 (dd, J=11.8, 6.5 Hz, 2H), 3.01 (s, 1H), 2.88-2.77 (m, 1H), 2.73-2.67 (m, 2H), 2.14-2.03 (m, 2H), 1.99 (dd, J=9.6, 5.3 Hz, 1H), 1.89-1.81 (m, 1H), 1.81-1.71 (m, 2H), 1.71-1.61 (m, 3H).

### Example 2: 4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-8-yl)-6-methyl-5-(trifluoromethyl)-1H-indazole-3-carbonitrile (Ex-2)

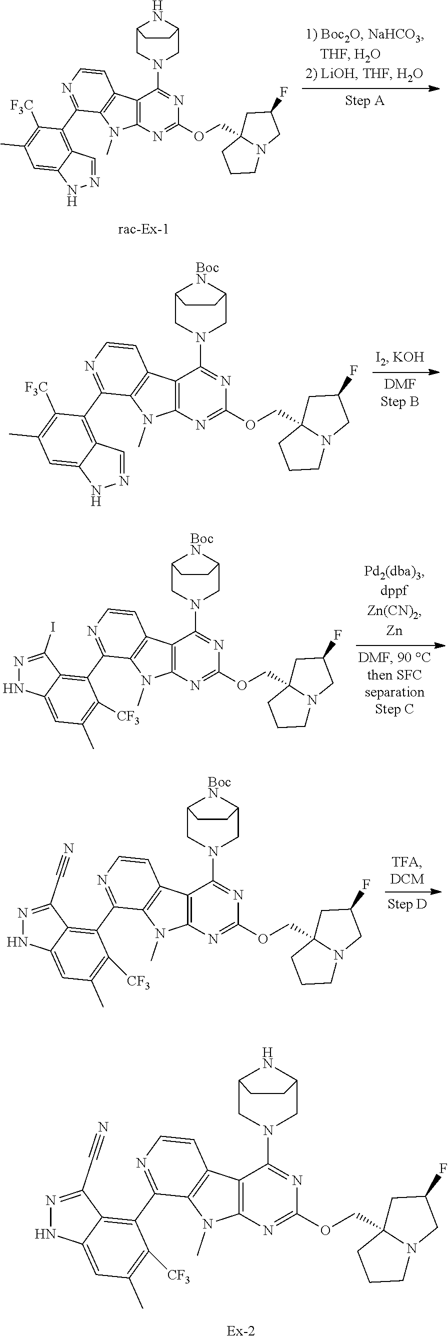

### Step A: Tert-butyl (1R,5S)-3-(2-(((2R,7aS)-2-fluorotetrahydro-1H-pyrrolizin-7a(5H)-yl)methoxy)-9-methyl-8-(6-methyl-5-(trifluoromethyl)-1H-indazol-4-yl)-9H-pyrido[4′,3′:4,5]pyrrolo[2,3-d]pyrimidin-4-yl)-3,8-diazabicyclo[3.2.1]octane-8-carboxylate

To a solution of racemic 4-((1R,5S)-3,8-diazabicyclo[3.2.1]octan-3-yl)-2-(((2R,7aS)-2-fluorotetrahydro-1H-pyrrolizin-7a(5H)-yl)methoxy)-9-methyl-8-(6-methyl-5-(trifluoromethyl)-1H-indazol-4-yl)-9H-pyrido[4′,3′:4,5]pyrrolo[2,3-d]pyrimidine (rac-Ex-1) (195 mg, 0.300 mmol, mixture of atropisomers) in THF (3 mL) and water (3 mL) was added NaHCO₃ (101 mg, 1.201 mmol) and Boc₂O (0.070 mL, 0.300 mmol) and the reaction mixture was stirred at 25° C. for 2 h. The reaction mixture was diluted with H₂O (5 mL) and the mixture was extracted with 10% MeOH in DCM (30 mL). The combined organic phase was dried over anhydrous Na₂SO₄, filtered, and concentrated in vacuo. The residue was dissolved in THF (3 mL) and water (3 mL), and LiOH (21.6 mg, 0.900 mmol) was added to the mixture. The reaction mixture was stirred at 25° C. for 1 h. The reaction mixture was diluted with H₂O (5 mL) and the mixture was extracted with 10% MeOH in DCM (30 mL). The organic phase was dried over anhydrous Na₂SO₄, filtered, and concentrated in vacuo. The residue was purified by flash silica gel chromatography (petroleum ether:THF=1:2) to give tert-butyl (1R,5S)-3-(2-(((2R,7aS)-2-fluorotetrahydro-1H-pyrrolizin-7a(5H)-yl)methoxy)-9-methyl-8-(6-methyl-5-(trifluoromethyl)-1H-indazol-4-yl)-9H-pyrido[4′,3′:4,5]pyrrolo[2,3-d]pyrimidin-4-yl)-3,8-diazabicyclo[3.2.1]octane-8-carboxylate. MS (ESI) [M+H]⁺: m/z 750.

### Step B: Tert-butyl (1R,5S)-3-(2-(((2R,7aS)-2-fluorotetrahydro-1H-pyrrolizin-7a(5H)-yl)methoxy)-8-(3-iodo-6-methyl-5-(trifluoromethyl)-1H-indazol-4-yl)-9-methyl-9H-pyrido[4′,3′:4,5]pyrrolo[2,3-d]pyrimidin-4-yl)-3,8-diazabicyclo[3.2.1]octane-8-carboxylate

To a solution of tert-butyl (1R,5S)-3-(2-(((2R,7aS)-2-fluorotetrahydro-1H-pyrrolizin-7a(5H)-yl)methoxy)-9-methyl-8-(6-methyl-5-(trifluoromethyl)-1H-indazol-4-yl)-9H-pyrido[4′,3′:4,5]pyrrolo[2,3-d]pyrimidin-4-yl)-3,8-diazabicyclo[3.2.1]octane-8-carboxylate (120 mg, 0.160 mmol) in DMF (2 mL) was added KOH (27 mg, 0.48 mmol) and I₂ (162 mg, 0.640 mmol) at 25° C. and the mixture was stirred at 25° C. for 1 h. The reaction was quenched with sat. aq. Na₂SO₃ (3 mL) and the resulting mixture was extracted with EtOAc (3×20 mL). The combined organic phase was dried over anhydrous Na₂SO₄, filtered, and concentrated in vacuo. The residue was purified by flash silica gel chromatography (petroleum ether:THF=1:2) to give tert-butyl (1R,5S)-3-(2-(((2R,7aS)-2-fluorotetrahydro-1H-pyrrolizin-7a(5H)-yl)methoxy)-8-(3-iodo-6-methyl-5-(trifluoromethyl)-1H-indazol-4-yl)-9-methyl-9H-pyrido[4′,3′:4,5]pyrrolo[2,3-d]pyrimidin-4-yl)-3,8-diazabicyclo[3.2.1]octane-8-carboxylate. MS (ESI) [M+H]⁺: m/z 876.

### Step C: Tert-butyl (1R,5S)-3-(8-(3-cyano-6-methyl-5-(trifluoromethyl)-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate

To a mixture of tert-butyl (1R,5S)-3-(2-(((2R,7aS)-2-fluorotetrahydro-1H-pyrrolizin-7a(5H)-yl)methoxy)-8-(3-iodo-6-methyl-5-(trifluoromethyl)-1H-indazol-4-yl)-9-methyl-9H-pyrido[4′,3′:4,5]pyrrolo[2,3-d]pyrimidin-4-yl)-3,8-diazabicyclo[3.2.1]octane-8-carboxylate (120 mg, 0.137 mmol) in DMF (2 mL) was added zinc (18 mg, 0.27 mmol), zinc cyanide (80 mg, 0.69 mmol), dppf (15 mg, 0.027 mmol), and Pd₂(dba)₃ (13 mg, 0.014 mmol) at 25° C. The reaction was heated to 90° C. and stirred for 1 h under N₂. The reaction mixture was cooled to room temperature, diluted with EtOAc (30 mL), and the mixture was washed with H₂O (3×3 mL) and brine (3×2 mL). The organic phase was dried over anhydrous Na₂SO₄, filtered, and concentrated in vacuo. The residue was purified by preparative TLC (SiO₂, DCM:MeOH=10:1). The atropisomers of the purified material were separated by preparative-scale chiral SFC (Column C, 50% MeOH with 0.1% NH₄OH modifier) to afford tert-butyl (1R,5S)-3-(8-(3-cyano-6-methyl-5-(trifluoromethyl)-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate (the first eluting isomer from SFC). MS (ESI) [M+H]⁺: m/z 775.

### Step D: 4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-8-yl)-6-methyl-5-(trifluoromethyl)-1H-indazole-3-carbonitrile (Ex-2)

To a solution of tert-butyl (1R,5S)-3-(8-(3-cyano-6-methyl-5-(trifluoromethyl)-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate (15 mg, 0.019 mmol) in DCM (0.75 mL) was added TFA (0.25 mL, 3.24 mmol), and the mixture was stirred at 25° C. for 1 h. The mixture was concentrated in vacuo and the residue was purified by preparative HPLC (MeCN/H₂O, NH₃OH+NH₄HCO₃ modifier) to give 4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-8-yl)-6-methyl-5-(trifluoromethyl)-1H-indazole-3-carbonitrile (Ex-2). MS (ESI) [M+H]⁺: m/z 675. ¹H NMR (400 MHz, MeOH-d₄) δ 8.43 (d, J=5.5 Hz, 1H), 7.96-7.85 (m, 2H), 5.41-5.19 (m, 1H), 4.52 (br d, J=11.9 Hz, 2H), 4.33-4.27 (m, 1H), 4.25-4.16 (m, 1H), 3.80-3.62 (m, 4H), 3.30-3.23 (m, 2H), 3.21 (s, 1H), 3.13 (s, 3H), 3.03 (dt, J=5.6, 9.6 Hz, 1H), 2.78 (br d, J=1.5 Hz, 3H), 2.42-2.11 (m, 4H), 2.07-1.96 (m, 2H), 1.94-1.83 (m, 4H).

### Example 3: 4-((1R,5S)-3,8-diazabicyclo[3.2.1]octan-3-yl)-8-(5-(difluoromethyl)-6-methyl-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidine (Ex-3)

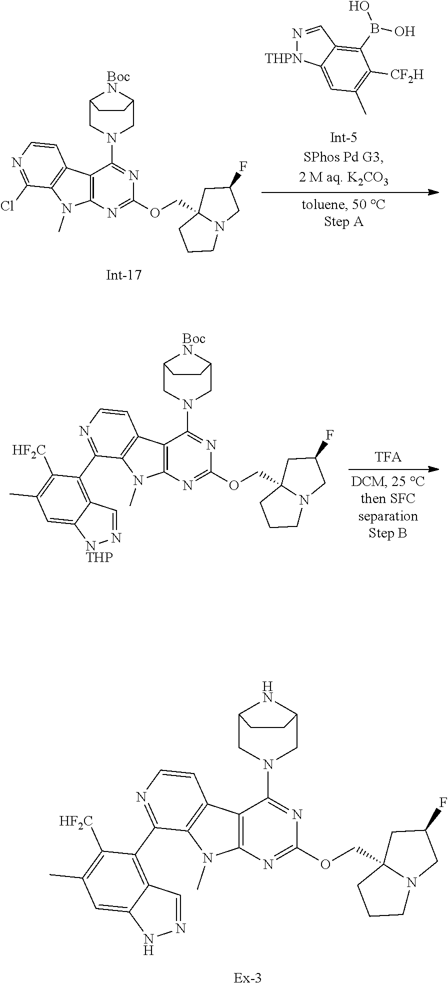

### Step A: Tert-butyl (1R,5S)-3-(8-(5-(difluoromethyl)-6-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate

To a solution of tert-butyl (1R,5S)-3-(8-chloro-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate (Int-17) (300 mg, 0.512 mmol) and (5-(difluoromethyl)-6-methyl-1-(tetrahydro-2H-pyran-2-yl)-1H-indazol-4-yl)boronic acid (Int-5) (238 mg, 0.768 mmol) in dry toluene (3 mL) was added SPhos Pd G3 (200 mg, 0.256 mmol) and K₂CO₃ (0.768 mL, 1.54 mmol, 2 M in H₂O) at 25° C. under N₂ atmosphere. After addition, the mixture was stirred at 50° C. for 4 h under N₂ atmosphere. After cooling the reaction mixture to room temperature, the mixture was diluted with water (5 mL) and extracted with EtOAc (3×5 mL). The combined organics were concentrated in vacuo and the residue was purified by flash silica gel chromatography (30 to 60% EtOAc in petroleum ether) to give tert-butyl (1R,5S)-3-(8-(5-(difluoromethyl)-6-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate. MS (ESI) [M+H]⁺: m/z 816.

### Step B: 4-((1R,5S)-3,8-diazabicyclo[3.2.1]octan-3-yl)-8-(5-(difluoromethyl)-6-methyl-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidine (Ex-3)

To a solution of tert-butyl (1R,5S)-3-(8-(5-(difluoromethyl)-6-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate (800 mg, 0.980 mmol) in DCM (9 mL) was added TFA (3 mL, 38.9 mmol) at 25° C., and the reaction mixture was stirred at 25° C. for 3 h. The reaction mixture was concentrated in vacuo, and the residue was purified by preparative HPLC (MeCN/H₂O w/TFA modifier). The atropisomers were separated by preparative SFC (Column D, 40% MeOH with 0.1% NH₄OH modifier) to afford 4-((1R,5S)-3,8-diazabicyclo[3.2.1]octan-3-yl)-8-(5-(difluoromethyl)-6-methyl-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidine (Ex-3, the second eluting isomer from SFC). MS (ESI) [M+H]⁺: m/z 632. ¹H NMR (400 MHz, MeOH-d₄) δ8.35 (d, J=5.5 Hz, 1H), 7.78 (d, J=5.6 Hz, 1H), 7.56 (s, 1H), 7.36 (s, 1H), 6.64-6.25 (m, 1H), 5.35-5.02 (m, 1H), 4.47 (br dd, J=4.6, 11.7 Hz, 2H), 4.22-4.13 (m, 1H), 4.11-4.03 (m, 1H), 3.65-3.51 (m, 4H), 3.18-3.05 (m, 3H), 2.98-2.85 (m, 4H), 2.66 (s, 3H), 2.26-2.08 (m, 2H), 2.08-1.99 (m, 1H), 1.94-1.83 (m, 2H), 1.81-1.69 (m, 5H).

### Example 4: 4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-8-yl)-5-(difluoromethyl)-6-methyl-1H-indazole-3-carbonitrile (Ex-4)

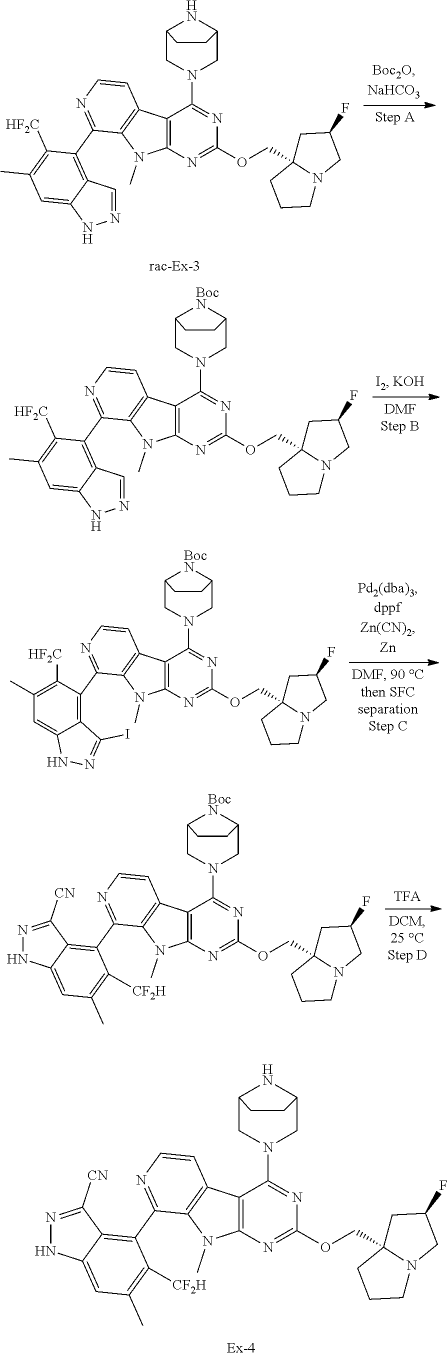

### Step A: Tert-butyl (1R,5S)-3-(8-(5-(difluoromethyl)-6-methyl-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate

To a solution of racemic 4-((1R,5S)-3,8-diazabicyclo[3.2.1]octan-3-yl)-8-(5-(difluoromethyl)-6-methyl-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidine (rac-Ex-3) (400 mg, 0.633 mmol, mixture of atropisomers) in THF (3 mL) and water (3 mL) was added sodium bicarbonate (160 mg, 1.90 mmol) and Boc₂O (0.147 mL, 0.633 mmol) and the reaction mixture was stirred at 25° C. for 2 h. The reaction mixture was extracted with DCM (20 mL) and the organic phase was dried over anhydrous Na₂SO₄, filtered, and concentrated in vacuo. The residue was purified by flash silica gel chromatography (petroleum ether:THF=1:2) to give tert-butyl (1R,5S)-3-(8-(5-(difluoromethyl)-6-methyl-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate. MS (ESI) [M+H]⁺: m/z 732.

### Step B: Tert-butyl (1R,5S)-3-(8-(5-(difluoromethyl)-3-iodo-6-methyl-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate

To a solution of tert-butyl (1R,5S)-3-(8-(5-(difluoromethyl)-6-methyl-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate (200 mg, 0.273 mmol) in DMF (3 mL) was added KOH (46 mg, 0.820 mmol) and I₂ (277 mg, 1.09 mmol) at 25° C. and the mixture was stirred at 25° C. for 1 h. The reaction mixture was quenched with sat. aq. Na₂SO₃ (5 mL) and the resulting mixture was extracted with DCM (40 mL, contained 10% MeOH). The organic phase was dried over anhydrous Na₂SO₄, filtered, and concentrated in vacuo. The residue was purified by flash silica gel chromatography (petroleum ether:THF=1:2) to give tert-butyl (1R,5S)-3-(8-(5-(difluoromethyl)-3-iodo-6-methyl-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate. MS (ESI) [M+H]⁺: m/z 858.

### Step C: Tert-butyl (1R,5S)-3-(8-(3-cyano-5-(difluoromethyl)-6-methyl-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate

To a mixture of tert-butyl (1R,5S)-3-(8-(5-(difluoromethyl)-3-iodo-6-methyl-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate (150 mg, 0.175 mmol) in DMF (0.5 mL) was added zinc (23 mg, 0.35 mmol), zinc cyanide (103 mg, 0.874 mmol), dppf (20 mg, 0.035 mmol), and Pd₂(dba)₃ (16 mg, 0.017 mmol) at 25° C., and the reaction was heated to 90° C. and stirred for 1 h under N₂. The reaction mixture was diluted with EtOAc (30 mL), and the mixture was washed with H₂O (3×3 mL) and brine (3×2 mL). The organic phase was dried over anhydrous Na₂SO₄, filtered, and concentrated in vacuo. The residue was purified by preparative TLC (DCM:MeOH=10:1). The resulting atropisomers were separated by preparative SFC (Column C, 50% MeOH, 0.1% NH₄OH modifier) to afford tert-butyl (1R,5S)-3-(8-(3-cyano-5-(difluoromethyl)-6-methyl-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate (first eluting isomer from SFC). MS (ESI) [M+H]⁺: m/z 757.

### Step D: 4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)-9-methyl-9H-pyrido[4′3′:4,5]pyrrolo[2,3-d]pyrimidin-8-yl)-5-(difluoromethyl)-6-methyl-1H-indazole-3-carbonitrile (Ex-4)

To a mixture of tert-butyl (1R,5S)-3-(8-(3-cyano-5-(difluoromethyl)-6-methyl-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate (25 mg, 0.033 mmol) in DCM (1 mL) was added TFA (0.3 mL, 3.89 mmol) at 25° C. and the reaction was stirred at 25° C. for 1 h. The reaction mixture was concentrated in vacuo and the residue was dissolved in MeOH (1 mL). The mixture was purified by preparative-scale HPLC (MeCN/H₂O w/NH₄OH+NH₄HCO₃ modifier)) to give 4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-8-yl)-5-(difluoromethyl)-6-methyl-1H-indazole-3-carbonitrile (Ex-4). MS (ESI) [M+H]⁺: m/z 657. ¹H NMR (400 MHz, MeOD) δ 8.48 (d, J=5.6 Hz, 1H), 7.91 (d, J=5.6 Hz, 1H), 7.83 (s, 1H), 6.93-6.56 (m, 1H), 5.45-5.13 (m, 1H), 4.61 (br d, J=12.5 Hz, 1H), 4.52 (br d, J=12.3 Hz, 1H), 4.34-4.24 (m, 1H), 4.23-4.15 (m, 1H), 3.75-3.61 (m, 4H), 3.30-3.17 (m, 3H), 3.08 (s, 3H), 3.02 (dt, J=5.8, 9.5 Hz, 1H), 2.81 (s, 3H), 2.39-2.26 (m, 1H), 2.25-2.20 (m, 1H), 2.20-2.10 (m, 1H), 2.07-1.94 (m, 2H), 1.94-1.73 (m, 5H).

Example 5: 4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-8-yl)-2-amino-7-fluorobenzo[b]thiophene-3-carbonitrile (Ex-5)

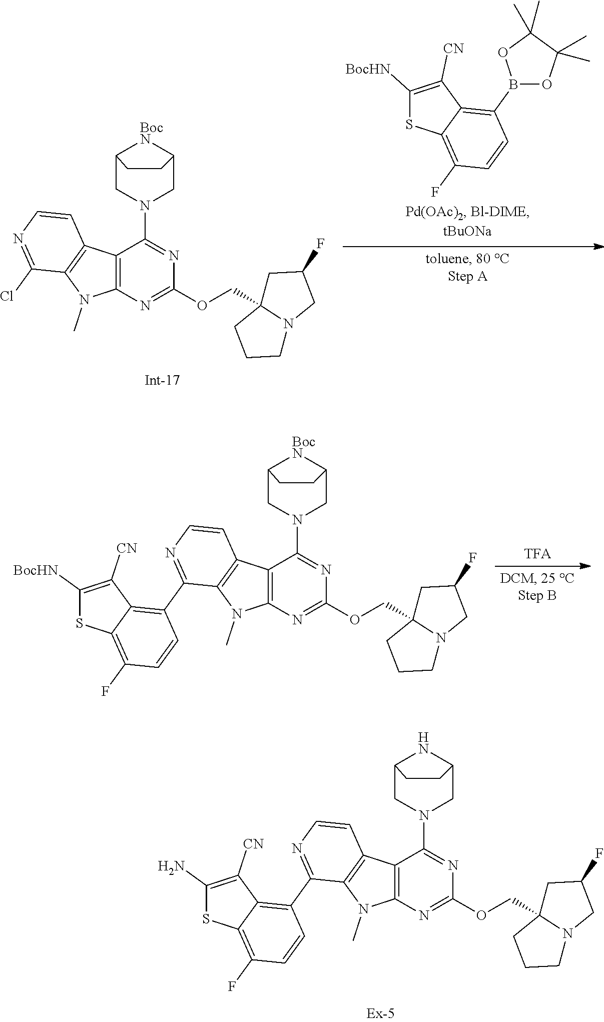 [SMILES: Cn1c2nc(OC[C@@]34CCCN3C[C@H](F)C4)nc(N3CC4CCC(C3)N4C(=O)OC(C)(C)C)c2c2ccnc(Cl)c21 ; CC(C)(C)OC(=O)Nc1sc2c(F)ccc(B3OC(C)(C)C(C)(C)O3)c2c1C#N ; Cn1c2nc(OC[C@@]34CCCN3C[C@H](F)C4)nc(N3CC4CCC(C3)N4C(=O)OC(C)(C)C)c2c2ccnc(-c3ccc(F)c4sc(NC(=O)OC(C)(C)C)c(C#N)c34)c21 ; Cn1c2nc(OC[C@@]34CCCN3C[C@H](F)C4)nc(N3CC4CCC(C3)N4)c2c2ccnc(-c3ccc(F)c4sc(N)c(C#N)c34)c21]

### Step A: Tert-butyl (1R,5S)-3-(8-(2-((tert-butoxycarbonyl)amino)-3-cyano-7-fluorobenzo[b]thiophen-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate

In a glovebox, a mixture of tert-butyl (1R,5S)-3-(8-chloro-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate (Int-17) (150 mg, 0.256 mmol), tert-butyl (3-cyano-4-(5,5-dimethyl-1,3,2-dioxaborinan-2-yl)-7-fluorobenzo[b]thiophen-2-yl)carbamate (166 mg, 0.409 mmol), palladium(II) acetate (28.7 mg, 0.128 mmol), rac-BI-DIME (85 mg, 0.256 mmol), and sodium tert-butoxide (73.8 mg, 0.768 mmol) was added to a vial with stir bar. Toluene (12 mL) was added via syringe and the reaction mixture was stirred at 80° C. for 12 h. The reaction mixture was diluted with EtOAc (10 mL) and H₂O (10 mL). The aqueous layer was separated and extracted with EtOAc (2×10 mL). The combined organic layers were washed with brine (10 mL), dried over Na₂SO₄, and filtered. The filtrate was concentrated under reduced pressure. The residue was purified by flash silica gel chromatography (0 to 26% THF in petroleum ether, column basified with Et₃N) to give tert-butyl (1R,5S)-3-(8-(2-((tert-butoxycarbonyl)amino)-3-cyano-7-fluorobenzo[b]thiophen-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate. MS (ESI) [M+H]⁺: m/z 842.

### Step B: 4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-8-yl)-2-amino-7-fluorobenzo[b]thiophene-3-carbonitrile (Ex-5)

To a solution of tert-butyl (1R,5S)-3-(8-(2-((tert-butoxycarbonyl)amino)-3-cyano-7-fluorobenzo[b]thiophen-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate (100 mg, 0.119 mmol) in DCM (1.5 mL) was added TFA (0.5 mL). The reaction mixture was stirred at 25° C. for 30 min. The mixture was concentrated under reduced pressure, and the residue was diluted with EtOAc (5 mL). The organic layer was washed with sat. aq. NaHCO₃ (2×1 mL). The organic layer was dried over Na₂SO₄ and filtered. The filtrate was concentrated under reduced pressure which was purified by preparative reverse-phase HPLC (MeCN/H₂O w/formic acid modifier) to give 4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-8-yl)-2-amino-7-fluorobenzo[b]thiophene-3-carbonitrile (Ex-5). MS (ESI) [M+H]⁺: m/z 642. ¹H NMR (400 MHz, MeOH-d₄) δ 8.43 (d, J=5.6 Hz, 1H), 7.85 (d, J=5.6 Hz, 1H), 7.41-7.44 (m, 1H), 7.08-7.13 (m, 1H), 5.58 (d, J=53.2 Hz, 1H), 4.60-4.88 (m, 4H), 4.23 (br s, 2H), 3.84-3.93 (m, 5H), 3.43-3.48 (m, 1H), 3.33 (br s, 3H), 2.61-2.75 (m, 2H), 2.32-2.34 (m, 3H), 2.09-2.15 (m, 5H).

### Example 6: 6-(4-((1R,5S)-3,8-diazabicyclo[3.2.1]octan-3-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-8-yl)-4-methyl-5-(trifluoromethyl)pyridin-2-amine (Ex-6)

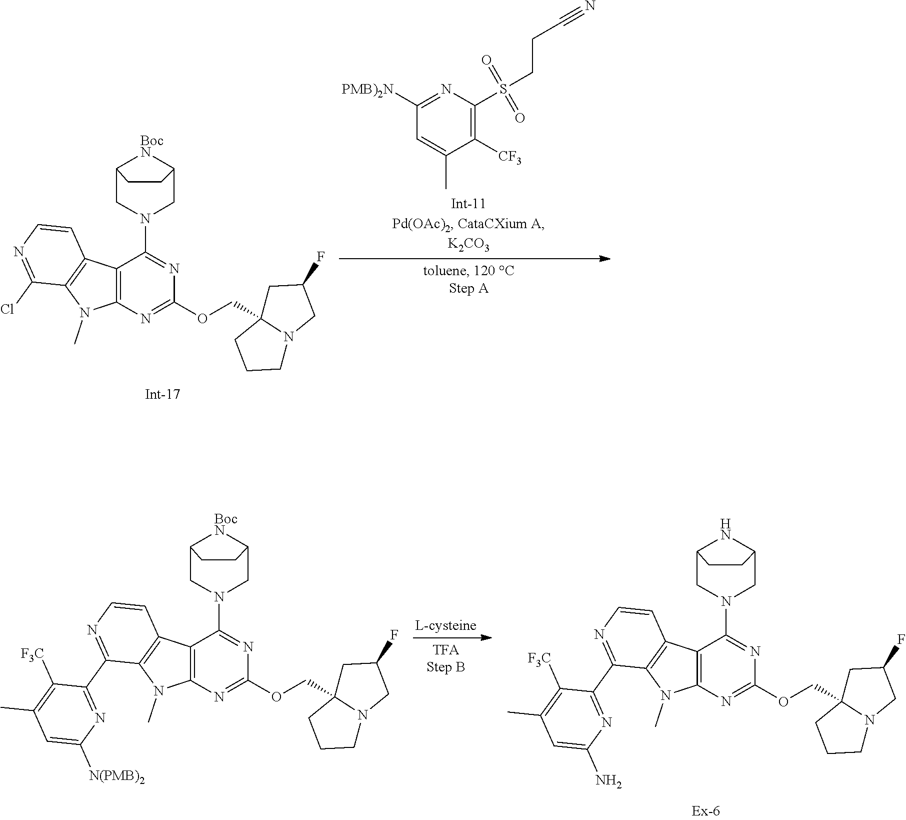

### Step A: Tert-butyl (1R,5S)-3-(8-(6-(bis(4-methoxybenzyl)amino)-4-methyl-3-(trifluoromethyl)pyridin-2-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate

To a solution of 3-((6-(bis(4-methoxybenzyl)amino)-4-methyl-3-(trifluoromethyl)pyridin-2-yl)sulfonyl)propanenitrile (Int-11) (130 mg, 0.244 mmol) in toluene (1.5 mL) was added K₂CO₃ (50.5 mg, 0.365 mmol), tert-butyl (1R,5S)-3-(8-chloro-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate (Int-17) (100 mg, 0.171 mmol), palladium(II) acetate (5.47 mg, 0.024 mmol) and di(1-adamantyl)-n-butylphosphine (17.5 mg, 0.049 mmol) at 20° C. in glove box. The mixture was stirred at 120° C. for 12 h. The mixture was diluted with water (3 mL) and extracted with EtOAc (3×5 mL), dried over sodium sulfate, filtered, and the solvent was evaporated under reduced pressure. The residue was purified by preparative TLC (SiO₂, DCM:MeOH=15:1) to give tert-butyl (1R,5S)-3-(8-(6-(bis(4-methoxybenzyl)amino)-4-methyl-3-(trifluoromethyl)pyridin-2-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate. MS (ESI) [M+H]⁺: m/z 966.

### Step B: 6-(4-((1R,5S)-3,8-diazabicyclo[3.2.1]octan-3-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-8-yl)-4-methyl-5-(trifluoromethyl)pyridin-2-amine (Ex-6)

To a solution of tert-butyl (1R,5S)-3-(8-(6-(bis(4-methoxybenzyl)amino)-4-methyl-3-(trifluoromethyl)pyridin-2-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate (82 mg, 0.085 mmol) in TFA (1 mL) was added L-cysteine (51.4 mg, 0.424 mmol) at 20° C. The mixture was stirred at 50° C. for 0.5 h. The reaction mixture was concentrated and diluted with sat. aq. NaHCO₃ (3 mL). The reaction mixture was extracted with EtOAc (3×6 mL), dried over sodium sulfate, filtered, and the solvent was evaporated under reduced pressure. The residue was purified by preparative reverse-phase HPLC (MeCN/H₂O w/NH₄HCO₃ modifier) to give 6-(4-((1R,5S)-3,8-diazabicyclo[3.2.1]octan-3-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-8-yl)-4-methyl-5-(trifluoromethyl)pyridin-2-amine (Ex-6). MS (ESI) [M+H]⁺: m/z 626. ¹H NMR (400 MHz, MeOH-d₄) δ 8.20 (d, J=5.6 Hz, 1H), 7.69 (d, J=5.6 Hz, 1H), 6.57 (s, 1H), 5.11-5.33 (m, 1H), 4.33-4.47 (m, 2H), 4.03-4.25 (m, 2H), 3.47-3.58 (m, 4H), 3.31 (s, 3H), 3.13-3.19 (m, 2H), 3.08 (s, 1H), 2.87-2.97 (m, 1H), 2.39 (d, J=1.3 Hz, 3H), 2.01-2.27 (m, 3H), 1.67-1.95 (m, 7H).

### Example 7: 1-(4-((1R,5S)-3,8-diazabicyclo[3.2.1]octan-3-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-8-yl)-8-ethynyl-7-fluoroisoquinolin-3-amine (Ex-7)

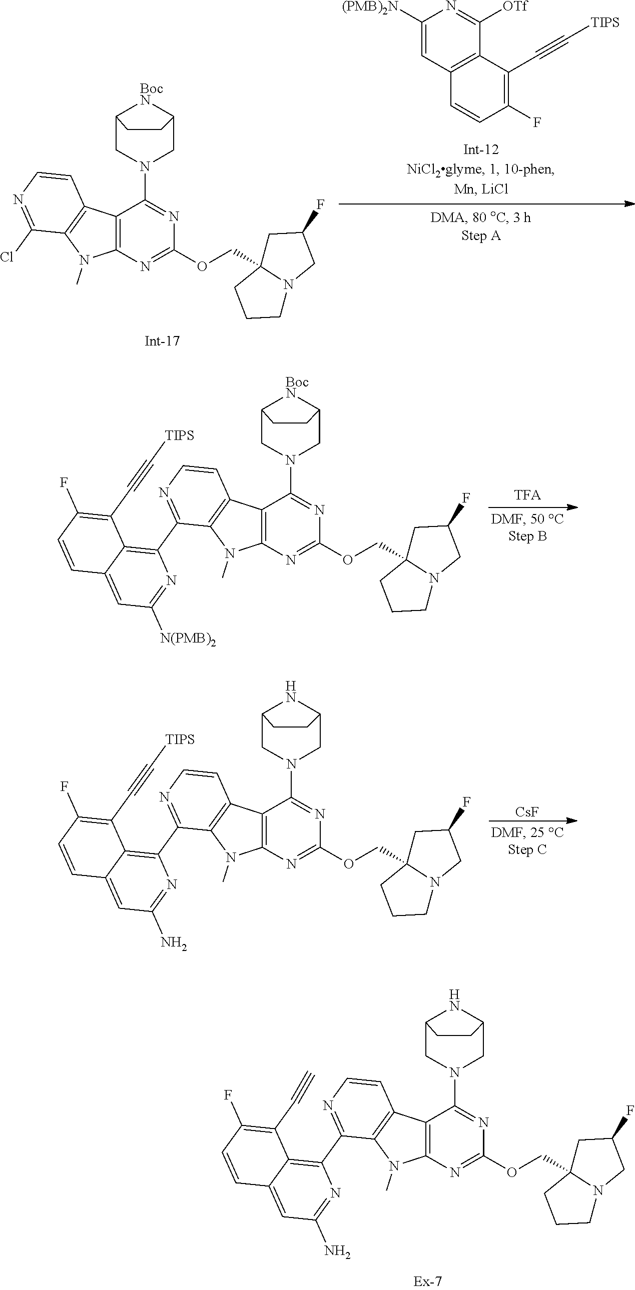

### Step A: Tert-butyl (1R,5S)-3-(8-(3-(bis(4-methoxybenzyl)amino)-7-fluoro-8-((triisopropylsilyl)ethynyl)isoquinolin-1-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate

To a solution of 1,10-phenanthroline (30 mg, 0.164 mmol) in DMA (3 mL) was added nickel(II) chloride ethylene glycol dimethyl ether complex (30.0 mg, 0.136 mmol) in a glovebox and the mixture was stirred at 50° C. for 1 h. Manganese dust (30.0 mg, 0.546 mmol), tert-butyl (1R,5S)-3-(8-chloro-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate (Int-17) (80 mg, 0.136 mmol), 3-(bis(4-methoxybenzyl)amino)-7-fluoro-8-((triisopropylsilyl)ethynyl)isoquinolin-1-yl trifluoromethanesulfonate (Int-12) (150 mg, 0.205 mmol) and lithium chloride (8.68 mg, 0.205 mmol) were added to the above nickel solution. The reaction was stirred at 80° C. for 3 h. The reaction was removed from the glovebox, diluted with EtOAc (30 mL), and washed with brine (4×2 mL). The organic layer was dried over sodium sulfate, filtered, and concentrated in vacuo. The residue was purified by flash silica gel chromatography (0 to 50% EtOAc in petroleum ether) to give tert-butyl (1R,5S)-3-(8-(3-(bis(4-methoxybenzyl)amino)-7-fluoro-8-((triisopropylsilyl)ethynyl)isoquinolin-1-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate. MS (ESI) [M/2+H]⁺: m/z 567.

### Step B: 1-(4-((1R,5S)-3,8-diazabicyclo[3.2.1]octan-3-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-8-yl)-7-fluoro-8-((triisopropylsilyl)ethynyl)isoquinolin-3-amine

To a solution of TFA (0.5 mL) in DCE (0.5 mL) was added tert-butyl (1R,5S)-3-(8-(3-(bis(4-methoxybenzyl)amino)-7-fluoro-8-((triisopropylsilyl)ethynyl)isoquinolin-1-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate (85 mg, 0.075 mmol) at 25° C. The reaction was stirred at 50° C. for 2 h. The reaction mixture was concentrated in vacuo and the residue was basified with sat. aq. NaHCO₃ (3 mL). The resulting mixture was extracted with DCM (3×5 mL). The combined organic phase was dried over anhydrous Na₂SO₄, filtered, and concentrated in vacuo to give 1-(4-((1R,5S)-3,8-diazabicyclo[3.2.1]octan-3-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-8-yl)-7-fluoro-8-((triisopropylsilyl)ethynyl)isoquinolin-3-amine. MS (ESI) [M+H]⁺: m/z 792.

### Step C: 1-(4-((1R,5S)-3,8-diazabicyclo[3.2.1]octan-3-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-8-yl)-8-ethynyl-7-fluoroisoquinolin-3-amine (Ex-7)

To a solution of 1-(4-((1R,5S)-3,8-diazabicyclo[3.2.1]octan-3-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-8-yl)-7-fluoro-8-((triisopropylsilyl)ethynyl)isoquinolin-3-amine (59 mg, 0.074 mmol) in DMF (0.5 mL) was added cesium fluoride (22.63 mg, 0.149 mmol) at 25° C. under N₂ atmosphere, and the mixture was stirred at 25° C. for 2 h. The reaction mixture was quenched with H₂O (3 mL) and the resulting mixture was extracted with DCM (3×5 mL). The combined organic phase was dried over anhydrous Na₂SO₄, filtered, and concentrated in vacuo to give crude reaction mixture which was purified by preparative reverse-phase HPLC (H₂O/MeCN w/NH₃ and NH₄HCO₃ modifier) to give 1-(4-((1R,5S)-3,8-diazabicyclo[3.2.1]octan-3-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-8-yl)-8-ethynyl-7-fluoroisoquinolin-3-amine (Ex-7). MS (ESI) [M+H]⁺: m/z 636. ¹H NMR (400 MHz, MeOH-d₄) δ 8.32 (d, J=5.6 Hz, 1H), 7.87 (d, J=5.6 Hz, 1H), 7.78 (dd, J=5.5, 9.3 Hz, 1H), 7.43 (t, J=9.1 Hz, 1H), 7.06 (s, 1H), 5.29 (d, J=52.8 Hz, 1H), 4.69-4.63 (m, 1H), 4.36-4.41 (m, 1H), 4.26 (dd, J=3.0, 10.4 Hz, 1H), 4.16 (dd, J=3.0, 10.4 Hz, 1H), 3.74 (dd, J=4.6, 11.9 Hz, 1H), 3.65 (br s, 2H), 3.50 (dd, J=4.8, 12.7 Hz, 1H), 3.27-3.19 (m, 3H), 3.19 (s, 3H), 3.16 (s, 1H), 2.96-3.02 (m, 1H), 2.36-2.18 (m, 2H), 2.11-2.14 (m, 1H), 2.02-1.79 (m, 6H), 1.75-1.65 (m, 1H).

### Example 8: 4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-8-yl)-5-ethynyl-6-fluoronaphthalen-2-ol (Ex-8)

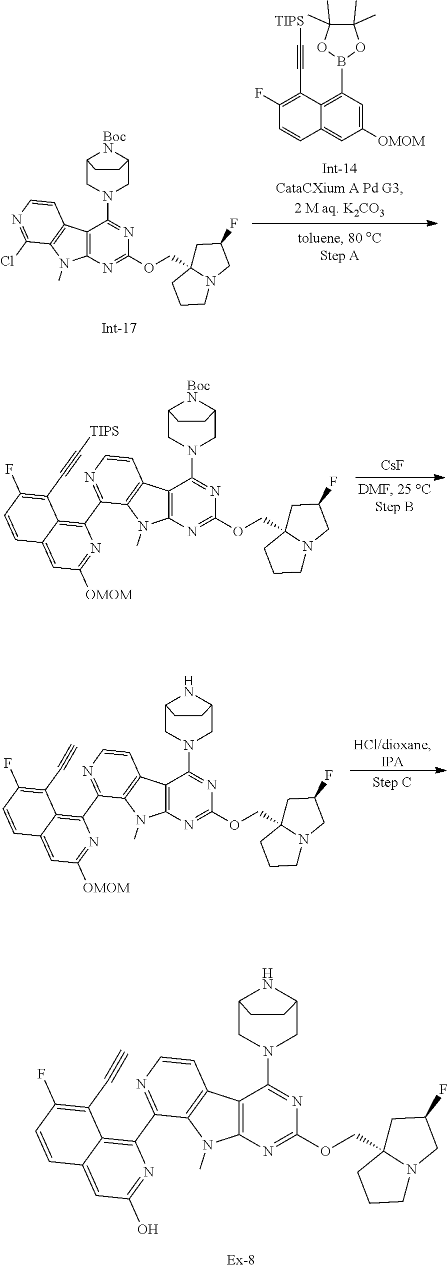

### Step A: Tert-butyl (1R,5S)-3-(8-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate

In a glovebox, a mixture of tert-butyl (1R,5S)-3-(8-chloro-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate (Int-17) (300 mg, 0.512 mmol), ((2-fluoro-6-(methoxymethoxy)-8-(4,4,5,5-tetramethyl-1,3,2-dioxaborolan-2-yl)naphthalen-1-yl)ethynyl)triisopropylsilane (Int-14) (420 mg, 0.819 mmol), cataXCium A Pd G3 (186 mg, 0.256 mmol), and K₂CO₃ (0.77 mL, 1.54 mmol, 2M in H₂O) in toluene (24 mL) was added to a flask with stir bar. The reaction mixture was fitted with a septum, removed from the glovebox, and stirred at 80° C. for 12 h. The reaction mixture was quenched with H₂O (15 mL) and EtOAc (15 mL). The aqueous layer was separated and extracted with EtOAc (2×15 mL). The combined organic layers were dried over Na₂SO₄ and filtered. The filtrate was concentrated under reduced pressure and purified by flash silica gel chromatography (0 to 48% THF in petroleum ether, column basified with Et₃N) to give tert-butyl (1R,5S)-3-(8-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate. MS (ESI) [M/2+H]⁺: m/z 469.

### Step B: Tert-butyl (1R,5S)-3-(8-(8-ethynyl-7-fluoro-3-(methoxymethoxy)naphthalen-1-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate

To a solution of tert-butyl (1R,5S)-3-(8-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate (105 mg, 0.112 mmol) in DMF (1 mL) was added CsF (85 mg, 0.56 mmol). The reaction mixture was stirred at 25° C. for 1 h. The reaction mixture was quenched with H₂O (5 mL). The aqueous layer was extracted with EtOAc (3×2 mL). The combined organic layers were dried over Na₂SO₄ and filtered. The filtrate was concentrated under reduced pressure to give tert-butyl (1R,5S)-3-(8-(8-ethynyl-7-fluoro-3-(methoxymethoxy)naphthalen-1-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate, which was used directly in the next step without further purification. MS (ESI) [M+H]⁺: m/z 780.

### Step C: 4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-8-yl)-5-ethynyl-6-fluoronaphthalen-2-ol (Ex-8)

Tert-butyl (1R,5S)-3-(8-(8-ethynyl-7-fluoro-3-(methoxymethoxy)naphthalen-1-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3,8-diazabicyclo[3.2.1]octane-8-carboxylate (80 mg, 0.126 mmol) was added to a vial with stir bar. Isopropanol (0.5 mL) and HCl (0.5 mL, 4 M in dioxane) were added via syringe and the reaction mixture was stirred at 25° C. for 3 h. The mixture was concentrated under reduced pressure. The residue was diluted with EtOAc (2 mL) and THF (2 mL). The organic layer was washed with sat. aq. NaHCO₃ (1 mL), dried over Na₂SO₄, and filtered. The filtrate was concentrated under reduced pressure and purified by preparative reverse-phase HPLC (MeCN/H₂O w/NH₄HCO₃ modifier) to give 4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-8-yl)-5-ethynyl-6-fluoronaphthalen-2-ol (Ex-8). MS (ESI) [M+H]⁺: m/z 636. ¹H NMR (400 MHz, MeOH-d₄) δ 8.30 (d, J=5.48 Hz, 1H), 7.89 (dd, J=5.54, 9.12 Hz, 1H), 7.82 (d, J=5.72 Hz, 1H), 7.41 (d, J=2.50 Hz, 1H), 7.33 (t, J=8.94 Hz, 1H), 7.22 (d, J=2.38 Hz, 1H), 5.20-5.37 (m, 1H), 4.67 (d, J=5.36 Hz, 1H), 4.36 (d, J=8.23 Hz, 1H), 4.22-4.29 (m, 1H), 4.10-4.18 (m, 1H), 3.71-3.78 (m, 1H), 3.65 (s, 2H), 3.47-3.51 (m, 1H), 3.15-3.24 (m, 3H), 3.11 (s, 3H), 2.96-3.05 (m, 2H), 2.18-2.34 (m, 2H), 2.11 (d, J=8.82 Hz, 1H), 1.81-2.00 (m, 6H), 1.70 (d, J=2.15 Hz, 1H).

The examples in the table below were synthesized using a similar procedure as described in the synthesis of Ex-8 by making the appropriate substitutions for starting material, intermediates, and/or reagents. Appropriate substitutions are available commercially, synthesized as described in the literature, synthesized using methods available to those skilled in the art, or synthesized as described herein.

| | | | | [M + |
|---|---|---|---|---|
| | Starting | | | H]⁺ |
| Ex | Material | Structure | Compound Name | Found |
| Ex- 9 | 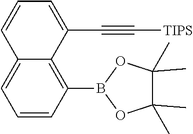 | 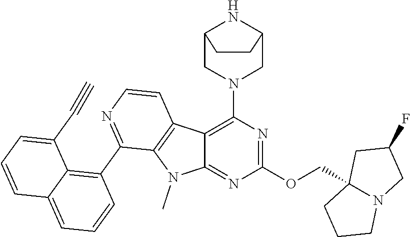 [SMILES: C#Cc1cccc2cccc(-c3nccc4c5c(N6CC7CCC(C6)N7)nc(OC[C@@]67CCCN6C[C@H](F)C7)nc5n(C)c34)c12] | 4-((1R,5S)-3,8- diazabicyclo[3.2.1] octan-3-yl)-8-(8- ethynylnaphthalen- 1-yl)-2-(((2R,7aS)- 2-fluoro- tetrahydro-1H- pyrrolizin-7a(5H)- yl)methoxy)-9- methyl-9H-py- rido[4′,3′:4,5]pyrro- lo[2,3- d]pyrimidine | 602 |
| Ex- 10 | 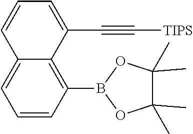 | 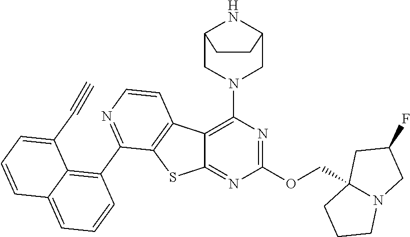 [SMILES: C#Cc1cccc2cccc(-c3nccc4c3sc3nc(OC[C@@]56CCCN5C[C@H](F)C6)nc(N5CC6CCC(C5)N6)c34)c12] | 4-((1R,5S)-3,8- diaza- bicyclo[3.2.1]oc- tan-3-yl)-8-(8- ethynylnaphthalen- 1-yl)-2- (((2R,7aS)-2-fluoro- tetrahydro-1H- pyrrolizin-7a(5H)- yl)methoxy)pyri- do[4′,3′:4,5]thi- eno[2,3-d]py- rimidine | 605 |

### Example 11: 4-((1R,5S)-3,8-diazabicyclo[3.2.1]octan-3-yl)-9-ethyl-2-(((2R,7aS)-2-fluorotetrahydro-1H-pyrrolizin-7a(5H)-yl)methoxy)-8-(6-methyl-5-(trifluoromethyl)-1H-indazol-4-yl)-9H-pyrido[4′,3′:4,5]pyrrolo[2,3-d]pyrimidine (Ex-11)

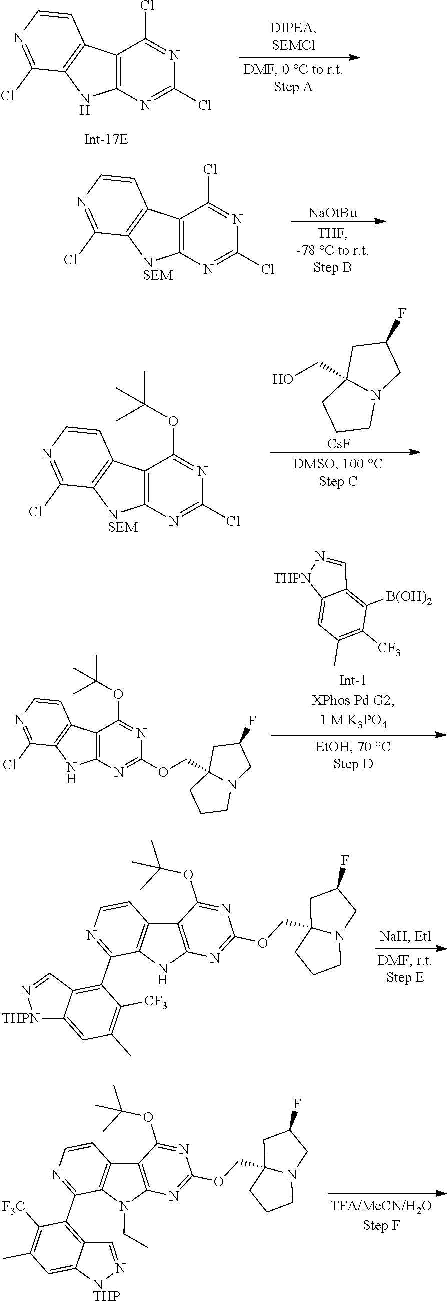

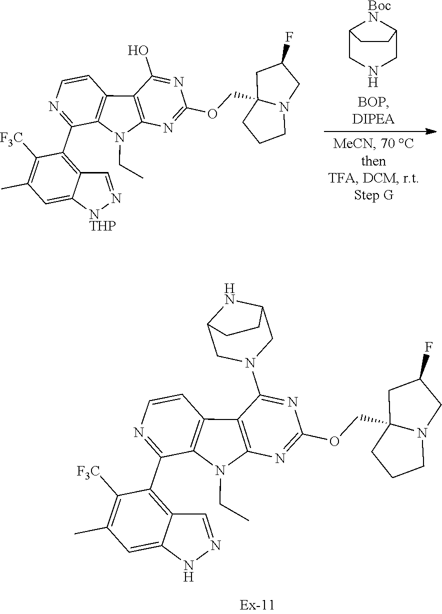

### Step A: 2,4,8-trichloro-9-((2-(trimethylsilyl)ethoxy)methyl)-9H-pyrido[4′,3′:4,5]pyrrolo[2,3-d]pyrimidine

N,N-diisopropylethylamine (2.5 mL, 15 mmol) followed immediately by (2-(chloromethoxy)ethyl)trimethylsilane (1.9 mL, 11 mmol) were added to a solution of 2,4,8-trichloro-9H-pyrido[4′,3′:4,5]pyrrolo[2,3-d]pyrimidine (Int-17E) (2.00 g, 7.31 mmol) in DMF (24 mL) at 0° C. The reaction mixture was allowed to warm to room temperature and stirred for 4 h. After 4 h, the reaction mixture was concentrated in vacuo. The product was purified by silica gel chromatography (0 to 60% EtOAc in hexane) to provide 2,4,8-trichloro-9-((2-(trimethylsilyl)ethoxy)methyl)-9H-pyrido[4′,3′:4,5]pyrrolo[2,3-d]pyrimidine. MS (ESI) [M+H]⁺ m/z 403, 405.

### Step B: 4-(tert-butoxy)-2,8-dichloro-9-((2-(trimethylsilyl)ethoxy)methyl)-9H-pyrido[4′,3′:4,5]pyrrolo[2,3-d]pyrimidine

Sodium tert-butoxide (3.5 mL, 7.0 mmol, 2 M in THF) was added to a solution of 2,4,8-trichloro-9-((2-(trimethylsilyl)ethoxy)methyl)-9H-pyrido[4′,3′:4,5]pyrrolo[2,3-d]pyrimidine (2.6 g, 6.4 mmol) in THF (64.3 mL) at −78° C. The reaction mixture was allowed to warm to room temperature and stirred overnight. The mixture was concentrated in vacuo and purified by silica gel chromatography (0 to 60% EtOAc in hexane) to provide 4-(tert-butoxy)-2,8-dichloro-9-((2-(trimethylsilyl)ethoxy)methyl)-9H-pyrido[4′,3′:4,5]pyrrolo[2,3-d]pyrimidine. MS (ESI) [M+H]⁺ m/z 441, 443.

### Step C: 4-(tert-butoxy)-8-chloro-2-(((2R,7aS)-2-fluorotetrahydro-1H-pyrrolizin-7a(5H)-yl)methoxy)-9H-pyrido[4′,3′:4,5]pyrrolo[2,3-d]pyrimidine

Cesium fluoride (1.6 g, 11 mmol) was added to a solution of 4-(tert-butoxy)-2,8-dichloro-9-((2-(trimethylsilyl)ethoxy)methyl)-9H-pyrido[4′,3′:4,5]pyrrolo[2,3-d]pyrimidine (800 mg, 1.81 mmol) and ((2R,7aS)-2-fluorotetrahydro-1H-pyrrolizin-7a(5H)-yl)methanol (577 mg, 3.62 mmol) in DMSO (9.0 mL). The reaction mixture was stirred at 100° C. overnight. The mixture was concentrated in vacuo. The product was purified by silica gel chromatography (0 to 100% (3:1 EtOH:EtOAc) in hexane with 0.1% Et₃N additive) to provide 4-(tert-butoxy)-8-chloro-2-(((2R,7aS)-2-fluorotetrahydro-1H-pyrrolizin-7a(5H)-yl)methoxy)-9H-pyrido[4′,3′:4,5]pyrrolo[2,3-d]pyrimidine. MS (ESI) [M+H]⁺ m/z 434.

### Step D: 4-(tert-butoxy)-2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidine

A mixture of 4-(tert-butoxy)-8-chloro-2-(((2R,7aS)-2-fluorotetrahydro-1H-pyrrolizin-7a(5H)-yl)methoxy)-9H-pyrido[4′,3′:4,5]pyrrolo[2,3-d]pyrimidine (190 mg, 0.436 mmol), XPhos Pd G2 (69 mg, 0.087 mmol), and (6-methyl-1-(tetrahydro-2H-pyran-2-yl)-5-(trifluoromethyl)-1H-indazol-4-yl)boronic acid (Int-1) (286 mg, 0.871 mmol) was sealed in a vial and purged with argon. Ethanol (3.0 mL) and potassium phosphate tribasic (870 μL, 0.87 mmol, 1M in H₂O) was added to the reaction mixture and the reaction mixture was stirred at 70° C. overnight. The reaction mixture was filtered and concentrated in vacuo. The product was purified by silica gel chromatography (0 to 100% EtOAc in hexane then 0 to 100% (3:1 EtOH:EtOAc) in EtOAc) to provide 4-(tert-butoxy)-2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidine. MS (ESI) [M+H]⁺ m/z 682.

### Step E: 4-(tert-butoxy)-9-ethyl-2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidine

Sodium hydride (13 mg, 0.32 mmol, 60 wt % in mineral oil) was added to a solution of 4-(tert-butoxy)-2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidine (110 mg, 0.160 mmol) in DMF (1600 μl). The reaction mixture was stirred for 10 min prior to the addition of ethyl iodide (26 μL, 0.32 mmol) to the reaction mixture. The reaction mixture was allowed to stir overnight at room temperature. The reaction mixture was concentrated in vacuo and purified by silica gel chromatography (0 to 100% (3:1 EtOH:EtOAc) in hexane with 0.1% Et₃N additive) to provide 4-(tert-butoxy)-9-ethyl-2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidine. MS (ESI) [M+H-t-Bu]⁺ m/z 654.

### Step F: 9-ethyl-2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-ol

TFA (28 μl) was added to a solution of 4-(tert-butoxy)-9-ethyl-2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidine (98 mg, 0.14 mmol) in acetonitrile (1.08 mL) and water (0.28 mL). The reaction mixture was stirred overnight at room temperature. The mixture was concentrated in vacuo to yield 9-ethyl-2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-ol, which was used directly in the next step without further purification. MS (ESI) [M+H]⁺ m/z 654.

### Step G: 4-((1R,5S)-3,8-diazabicyclo[3.2.1]octan-3-yl)-9-ethyl-2-(((2R,7aS)-2-fluorotetrahydro-1H-pyrrolizin-7a(5H)-yl)methoxy)-8-(6-methyl-5-(trifluoromethyl)-1H-indazol-4-yl)-9H-pyrido[4′,3′:4,5]pyrrolo[2,3-d]pyrimidine (Ex-11)

To 9-ethyl-2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-ol (344 μL, 0.069 mmol) in MeCN (344 μL) was added N,N-diisopropylethylamine (60.1 μL, 0.344 mmol) followed by BOP (45.7 mg, 0.103 mmol). Tert-butyl 3,8-diazabicyclo[3.2.1]octane-8-carboxylate (29.2 mg, 0.138 mmol) was added and the reaction was stirred at 70° C. for 2 h. After 2 h, the reaction was filtered, and the filtrate was concentrated in vacuo. The mixture was redissolved in DCM (344 μL), treated with TFA (344 μL) and stirred at room temperature for 4 h. The reaction was concentrated in vacuo and redissolved in DMSO (1 mL). This solution was purified via preparative reverse-phase HPLC (MeCN/H₂O w/TFA modifier) to give 4-((1R,5S)-3,8-diazabicyclo[3.2.1]octan-3-yl)-9-ethyl-2-(((2R,7aS)-2-fluorotetrahydro-1H-pyrrolizin-7a(5H)-yl)methoxy)-8-(6-methyl-5-(trifluoromethyl)-1H-indazol-4-yl)-9H-pyrido[4′,3′:4,5]pyrrolo[2,3-d]pyrimidine (Ex-11) isolated as a TFA salt. MS (ESI) [M+H]⁺: m/z 664. ¹H NMR (499 MHz, d⁶-DMSO) δ 10.68 (s, 1H), 9.28 (s, 1H), 9.02 (s, 2H), 8.51 (s, 1H), 7.80 (s, 1H), 5.63 (s, 1H), 5.53 (s, 1H), 4.66-4.47 (m, 3H), 4.26 (s, 2H), 3.92-3.82 (m, 2H), 3.81-3.72 (m, 2H), 3.72-3.62 (m, 2H), 2.70 (s, 3H), 2.67-2.62 (m, 1H), 2.39-2.35 (m, 1H), 2.34-2.25 (m, 1H), 2.25-2.09 (m, 2H), 2.10-1.84 (m, 5H), 0.72-0.61 (m, 3H).

### Example 12: 4-((1R,5S)-3,8-diazabicyclo[3.2.1]octan-3-yl)-2-(((2R,7aS)-2-fluorotetrahydro-1H-pyrrolizin-7a(5H)-yl)methoxy)-8-(6-methyl-5-(trifluoromethyl)-1H-indazol-4-yl)pyrido[4′,3′:4,5]thieno[2,3-d]pyrimidine (Ex-12)

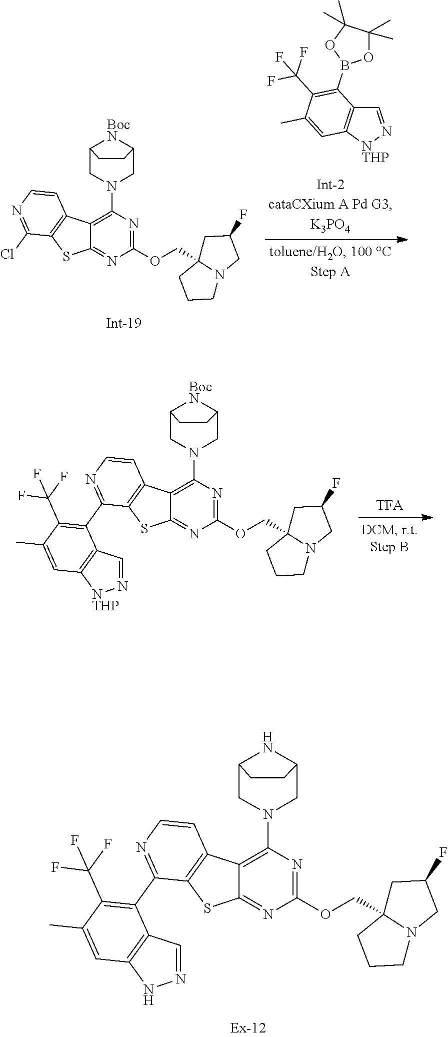

### Step A: Tert-butyl (1R,5S)-3-(2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)pyrido[4′,3′:4,5]thieno[2,3-d]pyrimidin-4-yl)-3,8-diazabicyclo[3.2.1]octane-8-carboxylate

A vial with stir bar containing tert-butyl (1R,5S)-3-(8-chloro-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate (Int-19) (35 mg, 0.059 mmol), 6-methyl-1-(tetrahydro-2H-pyran-2-yl)-4-(4,4,5,5-tetramethyl-1,3,2-dioxaborolan-2-yl)-5-(trifluoromethyl)-1H-indazole (Int-2) (36 mg, 0.088 mmol), cataCXium A Pd G3 (17 mg, 0.18 mmol), and K₃PO₄ (297 uL, 0.29 mmol, 1M in H₂O) in toluene (1 mL) was evacuated and refilled with N₂ (3 times). The mixture was heated to 100° C. and stirred under N₂ atmosphere for 12 h. After cooling to room temperature, the mixture was partitioned between EtOAc and water. The organic phase was washed with brine, dried over sodium sulfate, filtered, and concentrated. The crude residue was purified via silica gel chromatography (0 to 25% MeOH in EtOAc) to give tert-butyl (1R,5S)-3-(2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)pyrido[4′,3′:4,5]thieno[2,3-d]pyrimidin-4-yl)-3,8-diazabicyclo[3.2.1]octane-8-carboxylate. MS (ESI): m/z (M+H)⁺ 837.

### Step B: 4-((1R,5S)-3,8-diazabicyclo[3.2.1]octan-3-yl)-2-(((2R,7aS)-2-fluorotetrahydro-1H-pyrrolizin-7a(5H)-yl)methoxy)-8-(6-methyl-5-(trifluoromethyl)-1H-indazol-4-yl)pyrido[4′,3′:4,5]thieno[2,3-d]pyrimidine (Ex-12)

To a stirred solution of tert-butyl (1R,5S)-3-(2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)pyrido[4′,3′:4,5]thieno[2,3-d]pyrimidin-4-yl)-3,8-diazabicyclo[3.2.1]octane-8-carboxylate (25 mg, 0.029 mmol) in DCM (1.0 mL) was added TFA (500 μL) and the reaction mixture was stirred at room temperature for 30 min. The reaction was concentrated in vacuo and residue was partitioned between sat. aq. sodium bicarbonate and chloroform. The organic phase was washed with brine, dried over sodium sulfate, filtered, and concentrated. The crude residue was purified via flash NH-silica gel chromatography (0% to 10% MeOH in CHCl₃) to give 4-((1R,5S)-3,8-diazabicyclo[3.2.1]octan-3-yl)-2-(((2R,7aS)-2-fluorotetrahydro-1H-pyrrolizin-7a(5H)-yl)methoxy)-8-(6-methyl-5-(trifluoromethyl)-1H-indazol-4-yl)pyrido[4′,3′:4,5]thieno[2,3-d]pyrimidine (Ex-12) MS (ESI): (M+H)⁺ 653. ¹H NMR (400 MHz, CDCl₃) δ=10.35-9.86 (m, 1H), 8.73 (d, J=5.6 Hz, 1H), 7.60 (d, J=5.5 Hz, 1H), 7.56 (s, 1H), 7.49 (s, 1H), 5.41-5.16 (m, 1H), 4.30-4.20 (m, 3H), 4.11-4.05 (m, 1H), 3.71-3.54 (m, 4H), 3.32-3.24 (m, 2H), 3.23-3.10 (m, 1H), 3.05-2.92 (m, 1H), 2.75 (s, 3H), 2.38-2.27 (m, 1H), 2.27-2.13 (m, 3H), 2.02-1.74 (m, 6H).

The examples in the table below were synthesized using a similar procedure as described in the synthesis of Ex-12 by making the appropriate substitutions for starting material, intermediates, and/or reagents. Appropriate substitutions are available commercially, synthesized as described in the literature, synthesized using methods available to those skilled in the art, or synthesized as described herein.

| | Starting | | | [M + H]⁺ |
|---|---|---|---|---|
| Ex. | Material | Structure | Compound Name | Found |
| Ex-13 | 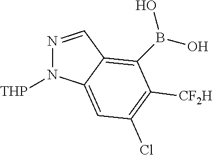 Int-3 | 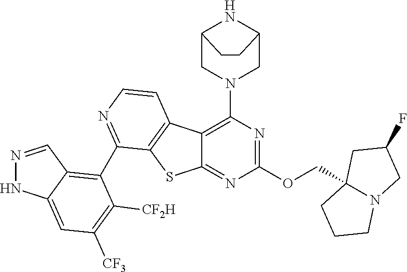 [SMILES: FC(F)c1c(C(F)(F)F)cc2[nH]ncc2c1-c1nccc2c1sc1nc(OC[C@@]34CCCN3C[C@H](F)C4)nc(N3CC4CCC(C3)N4)c12] | 4-((1R,5S)-3,8- diazabicyclo[3.2.1]octan-3-yl)-8- (6-chloro-5-(difluoromethyl)- 1H-indazol-4-yl)-2-(((2R,7aS)-2- fluorotetrahydro-1H-pyrrolizin- 7a(5H)- yl)methoxy)pyrido[4′,3′:4,5]thieno [2,3-d]pyrimidine | 655 |
| Ex-14 | Byproduct of Ex-13 | 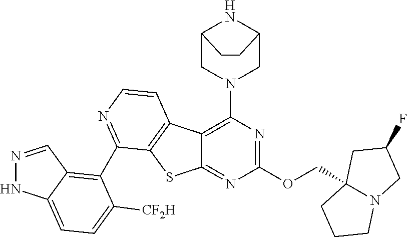 [SMILES: FC(F)c1ccc2[nH]ncc2c1-c1nccc2c1sc1nc(OC[C@@]34CCCN3C[C@H](F)C4)nc(N3CC4CCC(C3)N4)c12] | 4-((1R,5S)-3,8- diazabicyclo[3.2.1]octan-3-yl)-8- (5-(difluoromethyl)-1H-indazol- 4-yl)-2-(((2R,7aS)-2- fluorotetrahydro-1H-pyrrolizin- 7a(5H)- yl)methoxy)pyrido[4′,3′:4,5]thieno [2,3-d]pyrimidine | 621 |
| Ex-15 | 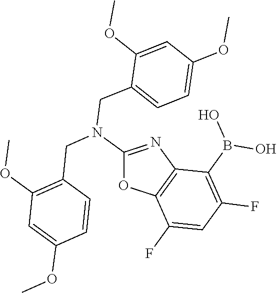 [SMILES: COc1ccc(CN(Cc2ccc(OC)cc2OC)c2nc3c(B(O)O)c(F)cc(F)c3o2)c(OC)c1] | 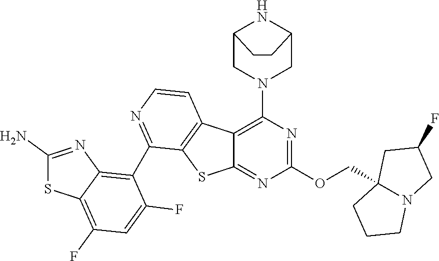 [SMILES: Nc1nc2c(-c3nccc4c3sc3nc(OC[C@@]56CCCN5C[C@H](F)C6)nc(N5CC6CCC(C5)N6)c34)c(F)cc(F)c2s1] | 4-(4-((1R,5S)-3,8- diazabicyclo[3.2.1]octan-3-yl)-2- (((2R,7aS)-2-fluorotetrahydro- 1H-pyrrolizin-7a(5H)- yl)methoxy)pyrido[4′,3′:4,5]thieno [2,3-d]pyrimidin-8-yl)-5,7- difluorobenzo[d]oxazol-2-amine | 623 |
| | Int-13 | | | |
| Ex-16 | 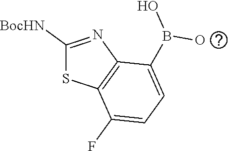 [SMILES: CC(C)(C)OC(=O)Nc1nc2c(B(O)O)ccc(F)c2s1] | 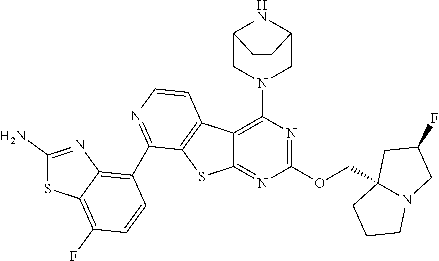 [SMILES: Nc1nc2c(-c3nccc4c3sc3nc(OC[C@@]56CCCN5C[C@H](F)C6)nc(N5CC6CCC(C5)N6)c34)ccc(F)c2s1] | 4-(4-((1R,5S)-3,8- diazabicyclo[3.2.1]octan-3-yl)-2- (((2R,7aS)-2-fluorotetrahydro- 1H-pyrrolizin-7a(5H)- yl)methoxy)pyrido[4′,3′:4,5]thieno [2,3-d]pyrimidin-8-yl)-7- fluorobenzo[d]thiazol-2-amine | 621 |
| Ex-17 | 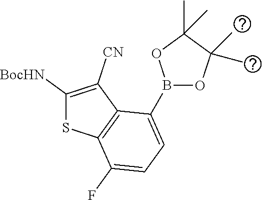 [SMILES: CC(C)(C)OC(=O)Nc1sc2c(F)ccc(B3OC(C)(C)C(C)(C)O3)c2c1C#N] | 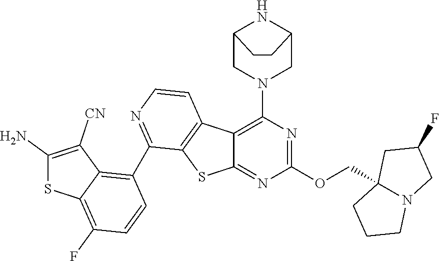 [SMILES: N#Cc1c(N)sc2c(F)ccc(-c3nccc4c3sc3nc(OC[C@@]56CCCN5C[C@H](F)C6)nc(N5CC6CCC(C5)N6)c34)c12] | 4-(4-((1R,5S)-3,8- diazabicyclo[3.2.1]octan-3-yl)-2- (((2R,7aS)-2-fluorotetrahydro- 1H-pyrrolizin-7a(5H)- yl)methoxy)pyrido[4′,3′:4,5]thieno [2,3-d]pyrimidin-8-yl)-2- amino-7- fluorobenzo[b] thiophene-3- carbonitrile | 645 |

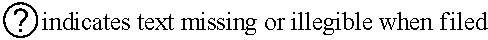

### Example 18: 4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)pyrido[4′,3′:4,5]thieno[2,3-d]pyrimidin-8-yl)-5-(difluoromethyl)-6-methyl-1H-indazole-3-carbonitrile (Ex-18)

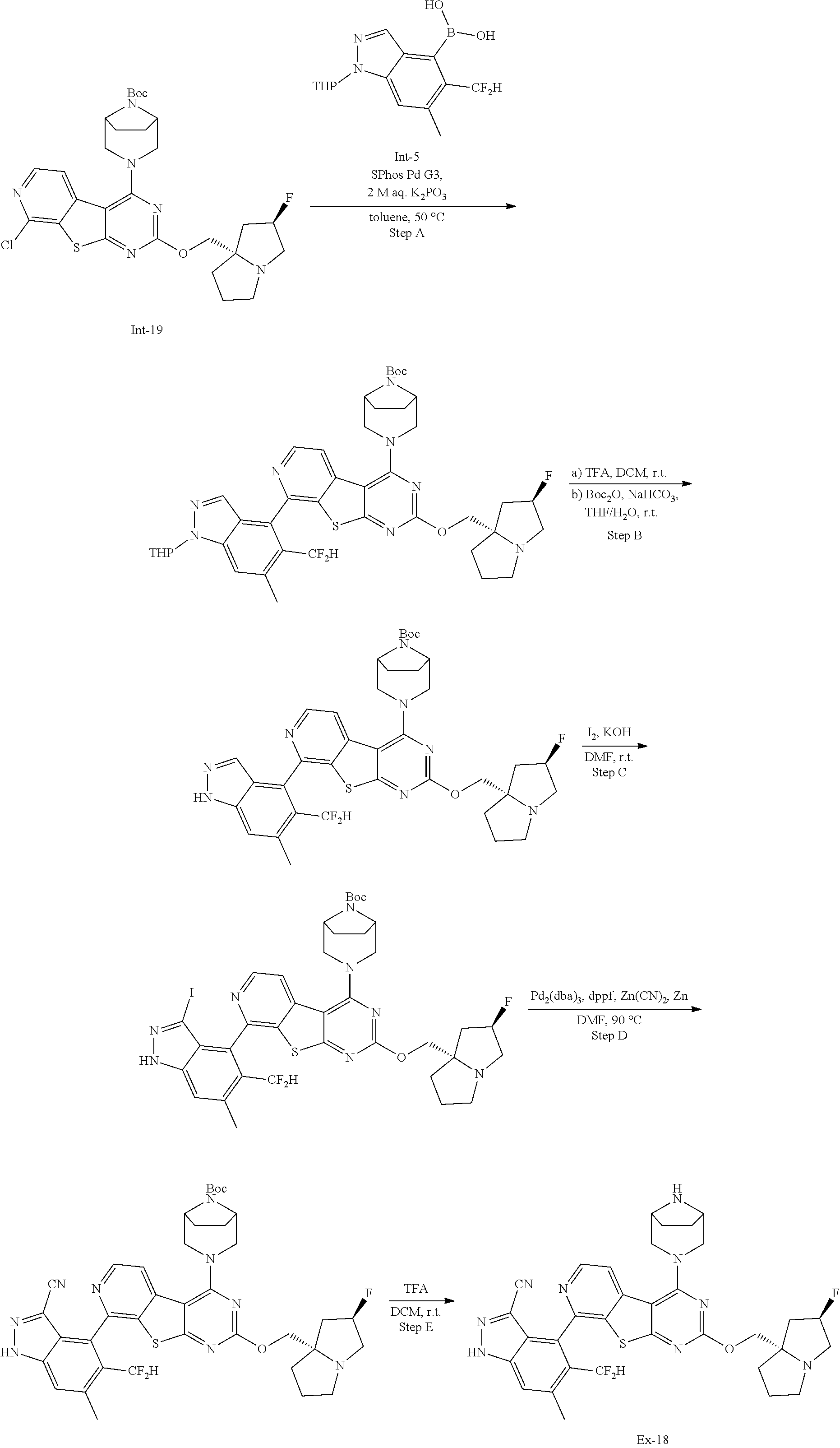

### Step A: Tert-butyl (1R,5S)-3-(8-(5-(difluoromethyl)-6-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate

To a solution of tert-butyl (1R,5S)-3-(8-chloro-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate (Int-19) (500 mg, 0.849 mmol) in toluene (12 mL) was added (5-(difluoromethyl)-6-methyl-1-(tetrahydro-2H-pyran-2-yl)-1H-indazol-4-yl)boronic acid (Int-5) (395 mg, 1.273 mmol), SPhos Pd G3 (331 mg, 0.424 mmol), and potassium carbonate (1.273 mL, 2.55 mmol, 2 M in H₂O) at 25° C. The reaction was placed under N₂ atmosphere and stirred at 50° C. for 3 h. The reaction was cooled to room temperature and diluted with EtOAc (30 mL) and washed with brine (10 mL). The organic layer was dried over Na₂SO₄, filtered, and concentrated in vacuo. The residue was purified by flash silica gel chromatography (0 to 50% THF in petroleum ether) to give tert-butyl (1R,5S)-3-(8-(5-(difluoromethyl)-6-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate. MS (ESI) [M+H]⁺: m/z 819.

### Step B: Tert-butyl (1R,5S)-3-(8-(5-(difluoromethyl)-6-methyl-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate

To a solution of tert-butyl (1R,5S)-3-(8-(5-(difluoromethyl)-6-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate (750 mg, 0.916 mmol) in DCM (10 mL) was added TFA (4 mL) at 25° C. The reaction was stirred at 25° C. for 1 h. The reaction mixture was concentrated in vacuo and the residue was dissolved in EtOAc (30 mL). The solution was washed with sat. aq. NaHCO₃ (20 mL) and brine (10 mL). The organic layer was dried over Na₂SO₄, filtered, and concentrated in vacuo to give 4-((1R,5S)-3,8-diazabicyclo[3.2.1]octan-3-yl)-8-(5-(difluoromethyl)-6-methyl-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidine, which was used directly in the next step without further purification. MS (ESI) [M+H]⁺: m/z 635. To a stirred solution of 4-((1R,5S)-3,8-diazabicyclo[3.2.1]octan-3-yl)-8-(5-(difluoromethyl)-6-methyl-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidine (581 mg, 0.915 mmol) in THF (6 mL) was added NaHCO₃ (231 mg, 2.75 mmol), H₂O (6 mL), and di-tert-butyl dicarbonate (0.211 mL, 0.915 mmol) at 25° C. The mixture was stirred at 25° C. for 16 h. The mixture was diluted with water (3 mL) and extracted with EtOAc (3×10 mL). The combined organic layers were dried over sodium sulfate, filtered, and the solvent was evaporated under reduced pressure. The residue was purified by flash silica gel chromatography (0 to 50% THF in petroleum ether) to give tert-butyl (1R,5S)-3-(8-(5-(difluoromethyl)-6-methyl-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate. MS (ESI) [M+H]⁺: m/z 735.

### Step C: Tert-butyl (1R,5S)-3-(8-(5-(difluoromethyl)-3-iodo-6-methyl-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate

To a solution of tert-butyl (1R,5S)-3-(8-(5-(difluoromethyl)-6-methyl-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate (600 mg, 0.817 mmol) in DMF (8 mL) was added KOH (137 mg, 2.450 mmol) and I₂ (829 mg, 3.27 mmol) at 25° C. The reaction was stirred at 25° C. for 1 h under N₂ atmosphere. The reaction was diluted with EtOAc (40 mL), washed with brine (5 mL) and sat. aq. Na₂SO₃ (2×5 mL). The organic layer was dried and concentrated in vacuo. The residue was purified by flash silica gel chromatography (0 to 50% THF in petroleum ether) to give tert-butyl (1R,5S)-3-(8-(5-(difluoromethyl)-3-iodo-6-methyl-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate. MS (ESI) [M+H]⁺: m/z 862.

### Step D: Tert-butyl (1R,5S)-3-(8-(3-cyano-5-(difluoromethyl)-6-methyl-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate

To a mixture of tert-butyl (1R,5S)-3-(8-(5-(difluoromethyl)-3-iodo-6-methyl-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate (200 mg, 0.232 mmol) in DMF (3 mL) was added zinc (30.4 mg, 0.465 mmol), zinc cyanide (136 mg, 1.16 mmol), dppf (25.8 mg, 0.046 mmol) and tris(dibenzylideneacetone)dipalladium(0) (21 mg, 0.023 mmol) at 25° C. The reaction was placed under a N₂ atmosphere and stirred at 90° C. for 30 min. The reaction was cooled to room temperature, diluted with EtOAc (20 mL), and filtered. The organic phase was washed with brine (3×2 mL), dried over anhydrous Na₂SO₄, filtered, and concentrated in vacuo. The residue was purified by flash silica gel chromatography (0 to 50% THF in petroleum ether) to give tert-butyl (1R,5S)-3-(8-(3-cyano-5-(difluoromethyl)-6-methyl-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate. MS (ESI) [M+H]⁺: m/z 760.

### Step E: 4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)pyrido[4′,3′:4,5]thieno[2,3-d]pyrimidin-8-yl)-5-(difluoromethyl)-6-methyl-1H-indazole-3-carbonitrile (Ex-18)

To a solution of tert-butyl (1R,5S)-3-(8-(3-cyano-5-(difluoromethyl)-6-methyl-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate (70 mg, 0.092 mmol) in DCM (2 mL) was added TFA (0.5 mL, 6.49 mmol) at 25° C., and the reaction was stirred at 25° C. for 1 h. The reaction mixture was concentrated in vacuo. The residue was dissolved in MeOH (1 mL) and basified with aq. NH₄OH, and then purified by preparative HPLC (MeCN/H₂O w/NH₄COOH modifier) to give 4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)pyrido[4′,3′:4,5]thieno[2,3-d]pyrimidin-8-yl)-5-(difluoromethyl)-6-methyl-1H-indazole-3-carbonitrile (Ex-18). MS (ESI) [M+H]⁺: m/z 660. ¹H NMR (400 MHz, MeOD) δ 8.75 (d, J=5.7 Hz, 1H), 7.89-7.76 (m, 2H), 6.87-6.53 (m, 1H), 5.41-5.19 (m, 1H), 4.44-4.33 (m, 1H), 4.31-4.26 (m, 1H), 4.24-4.17 (m, 2H), 3.70 (br t, J=15.6 Hz, 2H), 3.61 (br s, 2H), 3.27 (br d, J=7.4 Hz, 2H), 3.19 (s, 1H), 3.03 (dt, J=5.8, 9.4 Hz, 1H), 2.79 (s, 3H), 2.38-2.19 (m, 2H), 2.17-2.08 (m, 1H), 2.07-1.95 (m, 2H), 1.94-1.84 (m, 1H), 1.83-1.68 (m, 4H).

The example in the table below was synthesized using a similar procedure as described in the synthesis of Ex-18 by making the appropriate substitutions for starting material, intermediates, and/or reagents. Appropriate substitutions are available commercially, synthesized as described in the literature, synthesized using methods available to those skilled in the art, or synthesized as described herein.

| | | | | [M + |
|---|---|---|---|---|
| | Starting | | | H]⁺ |
| Ex. | Material | Structure | Compound Name | Found |
| Ex- 19 | 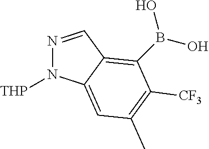 Int-1 | 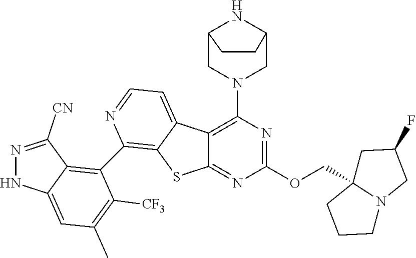 [SMILES: Cc1cc2[nH]nc(C#N)c2c(-c2nccc3c2sc2nc(OC[C@@]45CCCN4C[C@H](F)C5)nc(N4CC5CCC(C4)N5)c23)c1C(F)(F)F] | 4-(4-((1R,5S)-3,8- diazabicy- clo[3.2.1]oc- tan-3-yl)-2- (((2R,7aS)-2-fluoro- tetrahydro-1H- pyrrolizin-7a(5H)- yl)methoxy)pyri- do[4′,3′:4,5]thieno [2,3-d]pyrimidin-- 8-yl)-6-methyl-5- (trifluoromethyl)-- 1H-indazole-3- carbonitrile | 678 |

### Example 20: 4-((1R,5S)-3,8-diazabicyclo[3.2.1]octan-3-yl)-8-(6-chloro-5-cyclopropyl-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidine (Ex-20)

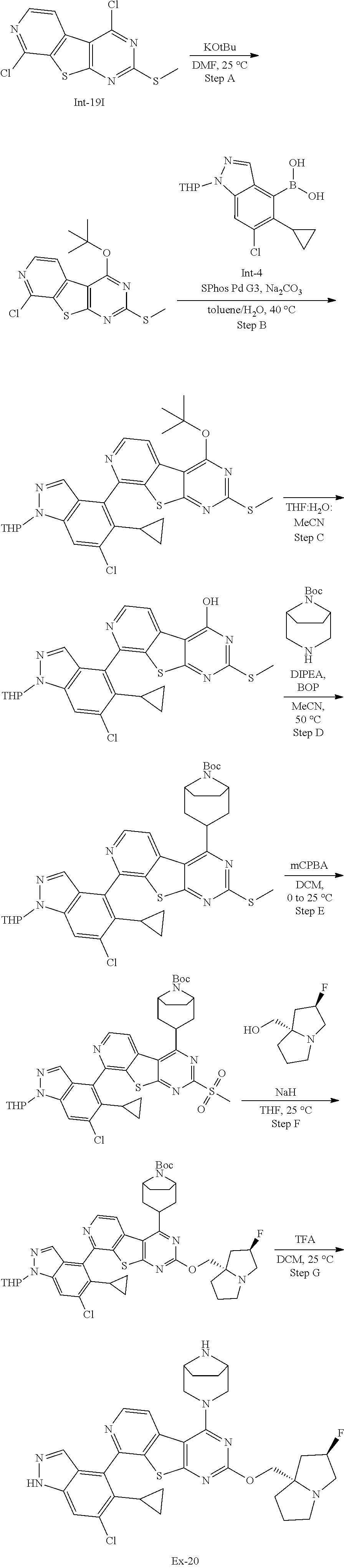

### Step A: 4-(tert-butoxy)-8-chloro-2-(methylthio)pyrido[4′,3′:4,5]thieno[2,3-d]pyrimidine

To a solution of 4,8-dichloro-2-(methylthio)pyrido[4′,3′:4,5]thieno[2,3-d]pyrimidine (Int-19I) (1 g, 3.31 mmol) in DMF (10 mL) was added potassium tert-butoxide (3.47 mL, 3.47 mmol, 1M in THF) at 25° C. The reaction mixture was stirred at 25° C. for 5 min under N₂ atmosphere. The reaction mixture was quenched with H₂O (30 mL), filtered, and the filter cake was collected and dried in vacuo to afford 4-(tert-butoxy)-8-chloro-2-(methylthio)pyrido[4′,3′:4,5]thieno[2,3-d]pyrimidine. MS (ESI) [M+H]⁺: m/z 340.

### Step B: 4-(tert-butoxy)-8-(6-chloro-5-cyclopropyl-1-(tetrahydro-2H-pyran-2-yl)-1H-indazol-4-yl)-2-(methylthio)pyrido[4′,3′:4,5]thieno[2,3-d]pyrimidine

To a solution of 4-(tert-butoxy)-8-chloro-2-(methylthio)pyrido[4′,3′:4,5]thieno[2,3-d]pyrimidine (500 mg, 1.47 mmol) in toluene (6 mL) was added (6-chloro-5-cyclopropyl-1-(tetrahydro-2H-pyran-2-yl)-1H-indazol-4-yl)boronic acid (Int-4) (495 mg, 1.545 mmol), Na₂CO₃ (3.68 mL, 7.36 mmol, 2M in H₂O), and SPhos Pd G3 (574 mg, 0.736 mmol) at 25° C. The reaction mixture was heated to 40° C. and stirred for 2 h under N₂ atmosphere. The reaction mixture was cooled to room temperature and quenched with H₂O (10 mL). The reaction mixture was washed with DCM (3×15 mL). The combined organics were dried over Na₂SO₄, filtered, and concentrated in vacuo. The crude material was purified by flash silica gel chromatography (0 to 35% EtOAc in petroleum ether, column basified with Et₃N) to give 4-(tert-butoxy)-8-(6-chloro-5-cyclopropyl-1-(tetrahydro-2H-pyran-2-yl)-1H-indazol-4-yl)-2-(methylthio)pyrido[4′,3′: 4,5]thieno[2,3-d]pyrimidine. MS (ESI) [M+H]⁺: m/z 580.

### Step C: 8-(6-chloro-5-cyclopropyl-1-(tetrahydro-2H-pyran-2-yl)-1H-indazol-4-yl)-2-(methylthio)pyrido[4′,3′:4,5]thieno[2,3-d]pyrimidin-4-ol

To a solution of TFA (1.0 mL, 13.0 mmol) in MeCN (40 mL) and H₂O (10 mL) was added 4-(tert-butoxy)-8-(6-chloro-5-cyclopropyl-1-(tetrahydro-2H-pyran-2-yl)-1H-indazol-4-yl)-2-(methylthio)pyrido[4′,3′:4,5]thieno[2,3-d]pyrimidine (3.3 g, 5.69 mmol) at 25° C., and the reaction mixture was stirred at 25° C. for 8 h under N₂ atmosphere. The reaction mixture was quenched with sat. aq. NaHCO₃ (20 mL) and extracted with EtOAc (3×30 mL). The organic layer was dried over Na₂SO₄, filtered, and concentrated in vacuo to give 8-(6-chloro-5-cyclopropyl-1-(tetrahydro-2H-pyran-2-yl)-1H-indazol-4-yl)-2-(methylthio)pyrido[4′,3′:4,5]thieno[2,3-d]pyrimidin-4-ol. MS (ESI) [M+H]⁺: m/z 524.

### Step D: Tert-butyl (1R,5S)-3-(8-(6-chloro-5-cyclopropyl-1-(tetrahydro-2H-pyran-2-yl)-1H-indazol-4-yl)-2-(methylthio)pyrido[4′,3′:4,5]thieno[2,3-d]pyrimidin-4-yl)-3,8-diazabicyclo[3.2.1]octane-8-carboxylate

To a solution of 8-(6-chloro-5-cyclopropyl-1-(tetrahydro-2H-pyran-2-yl)-1H-indazol-4-yl)-2-(methylthio)pyrido[4′,3′:4,5]thieno[2,3-d]pyrimidin-4-ol (1.07 g, 2.04 mmol) in MeCN (9 mL) was added BOP (1.81 g, 4.08 mmol) at 25° C. and the mixture was stirred at 25° C. for 30 min. Then to the reaction mixture was added a solution of tert-butyl (1R,5S)-3,8-diazabicyclo[3.2.1]octane-8-carboxylate (0.650 g, 3.06 mmol) and N,N-diisopropylethylamine (2.14 mL, 12.3 mmol) in MeCN (5 mL). The reaction mixture was heated to 50° C. and stirred for 1 h. The reaction mixture was cooled to room temperature and concentrated in vacuo. The residue was purified by flash silica gel chromatography (0 to 56% EtOAc in petroleum ether, column basified by Et₃N) to give tert-butyl (1R,5S)-3-(8-(6-chloro-5-cyclopropyl-1-(tetrahydro-2H-pyran-2-yl)-1H-indazol-4-yl)-2-(methylthio)pyrido[4′,3′:4,5]thieno[2,3-d]pyrimidin-4-yl)-3,8-diazabicyclo[3.2.1]octane-8-carboxylate. MS (ESI) [M+H]⁺: m/z 718.

### Step E: tert-butyl (1R,5S)-3-(8-(6-chloro-5-cyclopropyl-1-(tetrahydro-2H-pyran-2-yl)-1H-indazol-4-yl)-2-(methylsulfonyl)pyrido[4′,3′:4,5]thieno[2,3-d]pyrimidin-4-yl)-3,8-diazabicyclo[3.2.1]octane-8-carboxylate

To a solution of tert-butyl (1R,5S)-3-(8-(6-chloro-5-cyclopropyl-1-(tetrahydro-2H-pyran-2-yl)-1H-indazol-4-yl)-2-(methylthio)pyrido[4′,3′:4,5]thieno[2,3-d]pyrimidin-4-yl)-3,8-diazabicyclo[3.2.1]octane-8-carboxylate (600 mg, 0.835 mmol) in DCM (7 mL) was added mCPBA (339 mg, 1.671 mmol) at 0° C. The reaction was warmed to room temperature and stirred for 0.5 h. The reaction mixture was quenched with sat. aq. NaHCO₃ (5 mL) and Na₂SO₃ (10 mL), then extracted with DCM (3×10 mL). The organic layer was dried over Na₂SO₄, filtered, and concentrated in vacuo. The crude material was purified by flash silica gel chromatography (0 to 86% EtOAc in petroleum ether) to give tert-butyl (1R,5S)-3-(8-(6-chloro-5-cyclopropyl-1-(tetrahydro-2H-pyran-2-yl)-1H-indazol-4-yl)-2-(methylsulfonyl)pyrido[4′,3′:4,5]thieno[2,3-d]pyrimidin-4-yl)-3,8-diazabicyclo[3.2.1]octane-8-carboxylate. MS (ESI) [M+H]⁺: m/z 750.

### Step F: tert-butyl (1R,5S)-3-(8-(6-chloro-5-cyclopropyl-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate

To a solution of tert-butyl (1R,5S)-3-(8-(6-chloro-5-cyclopropyl-1-(tetrahydro-2H-pyran-2-yl)-1H-indazol-4-yl)-2-(methylsulfonyl)pyrido[4′,3′:4,5]thieno[2,3-d]pyrimidin-4-yl)-3,8-diazabicyclo[3.2.1]octane-8-carboxylate (120 mg, 0.160 mmol) and ((2R,7aS)-2-fluorotetrahydro-1H-pyrrolizin-7a(5H)-yl)methanol (50.9 mg, 0.320 mmol) in THF (2 mL) was added NaH (32.0 mg, 0.800 mmol) at 25° C. The reaction was stirred at 25° C. for 1 h. The reaction mixture was quenched with sat. aq. NH₄Cl (3 mL) and extracted with DCM (3×3 mL). The organic layer was dried over Na₂SO₄, filtered, and concentrated in vacuo. The crude was purified by preparative-scale TLC (SiO₂, EtOAc as eluent) to give tert-butyl (1R,5S)-3-(8-(6-chloro-5-cyclopropyl-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate. MS (ESI) [M+H]⁺: m/z 829.

### Step G: 4-((1R,5S)-3,8-diazabicyclo[3.2.1]octan-3-yl)-8-(6-chloro-5-cyclopropyl-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidine (Ex-20)

To a solution of TFA (0.5 mL, 6.49 mmol) in DCM (0.5 mL) was added tert-butyl (1R,5S)-3-(8-(6-chloro-5-cyclopropyl-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate (110 mg, 0.133 mmol) at 25° C. The reaction was stirred at 25° C. for 10 min. The solvent was removed by nitrogen flow, basified with sat. aq. NaHCO₃ (3 mL), and extracted with EtOAc (3×3 mL). The organic layer was dried over Na₂SO₄, filtered, and concentrated in vacuo. The residue was purified by preparative reverse phase HPLC (MeCN/H₂O w/NH₄OH+NH₄HCO₃ modifier) to give 4-((1R,5S)-3,8-diazabicyclo[3.2.1]octan-3-yl)-8-(6-chloro-5-cyclopropyl-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidine (Ex-20). MS (ESI) [M+H]⁺: m/z 645. ¹H NMR (400 MHz, MeOH-d₄) δ 8.60 (d, J=5.7 Hz, 1H), 7.74-7.65 (m, 2H), 7.46 (d, J=0.7 Hz, 1H), 5.27-5.06 (m, 1H), 4.28-4.06 (m, 4H), 3.71-3.46 (m, 4H), 3.16-3.08 (m, 2H), 3.06 (s, 1H), 2.89 (dt, J=5.5, 9.5 Hz, 1H), 2.29-1.95 (m, 3H), 1.92-1.81 (m, 3H), 1.81-1.61 (m, 5H), 0.70-0.52 (m, 1H), 0.38-0.22 (m, 1H), 0.08-−0.08 (m, 2H).

The examples in the table below were synthesized using a similar procedure as described in the synthesis of Ex-20 by making the appropriate substitutions for starting material, intermediates, and/or reagents. Appropriate substitutions are available commercially, synthesized as described in the literature, synthesized using methods available to those skilled in the art, or synthesized as described herein.

| | | | | [M + H]⁺ |
|---|---|---|---|---|
| Ex. | Starting Material | Structure | Compound Name | Found |
| Ex- 21 | 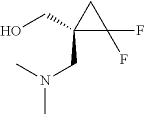 (R)-(1- ((dimethylamino)meth- yl)-2,2- difluorocyclopropyl) methanol | 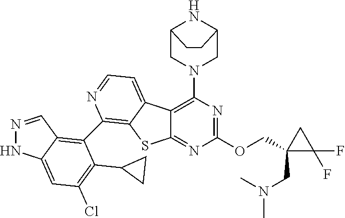 [SMILES: CN(C)C[C@@]1(COc2nc(N3CC4CCC(C3)N4)c3c(n2)sc2c(-c4c(C5CC5)c(Cl)cc5[nH]ncc45)nccc23)CC1(F)F] | 1-((1R)-1-(((4- ((1R,5S)-3,8- diazabicy- clo[3.2.1]octan-3- yl)-8-(6-chloro- 5-cyclopropyl- 1H-indazol-4- yl)pyrido [4′,3′:4,5]thieno [2,3-d]pyrimidin- 2-yl)oxy)methyl)- 2,2-difluoro- cyclopropyl)- N,N-dimethyl- | 651 |
| | | | methanamine | |
| Ex- 22 | 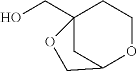 Int-15 | 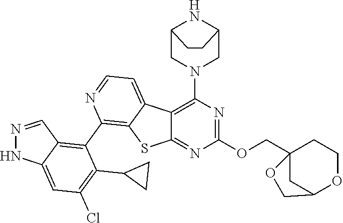 [SMILES: Clc1cc2[nH]ncc2c(-c2nccc3c2sc2nc(OCC45CCOC(CO4)C5)nc(N4CC5CCC(C4)N5)c23)c1C1CC1] | 2-((2,6- dioxabicy- clo[3.2.1]octan-5- yl)methoxy)-4- ((1R,5S)-3,8- diazabicy- clo[3.2.1]octan-3- yl)-8-(6-chloro- 5-cyclopropyl- 1H-indazol-4- yl)pyrido [4′,3′:4,5]thi- eno[2,3- d]pyrimidine | 630 |
| Ex- 23 | 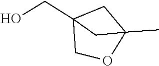 (1-methyl-2- oxabicyclo[2.1.1]hexan- 4-yl)methanol | 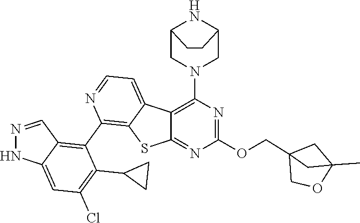 [SMILES: CC12CC(COc3nc(N4CC5CCC(C4)N5)c4c(n3)sc3c(-c5c(C6CC6)c(Cl)cc6[nH]ncc56)nccc34)(CO1)C2] | 4-((1R,5S)-3,8- diazabicy- clo[3.2.1]octan-3- yl)-8-(6-chloro-5- cyclopropyl-1H- indazol-4-yl)-2- (((1R,4r)-1- methyl-2-oxabicy- clo[2.1.1 ]hex- an-4- yl)methoxy)pyri- do[4′,3′:4,5]thi- eno[2,3-d]pyrimi- dine | 614 |
| Ex- 24 | 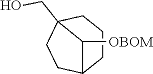 Int-16 | 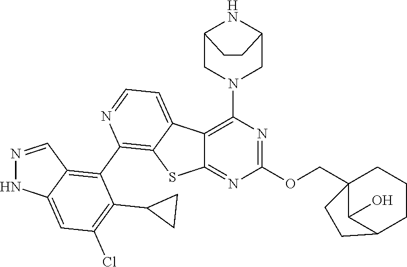 [SMILES: OC1C2CCCC1(COc1nc(N3CC4CCC(C3)N4)c3c(n1)sc1c(-c4c(C5CC5)c(Cl)cc5[nH]ncc45)nccc13)CC2] | 1-(((4-((1R,5S)- 3,8-diazabicy- clo[3.2.1]octan-3- yl)-8-(6-chloro-5- cyclopropyl-1H- indazol-4-yl)pyri- do[4′,3′:4,5]thi- eno[2,3- d]pyrimidin-2- yl)oxy)methyl)bi- cyclo[3.2.1]oc- tan-8-ol | 642 |

### Example 25: ((1R)-1-(((4-((1R,5S)-3,8-diazabicyclo[3.2.1]octan-3-yl)-8-(6-methyl-5-(trifluoromethyl)-1H-indazol-4-yl)pyrido[4′,3′:4,5]thieno[2,3-d]pyrimidin-2-yl)oxy)methyl)-2,2-difluorocyclopropyl)methanol (Ex-25)

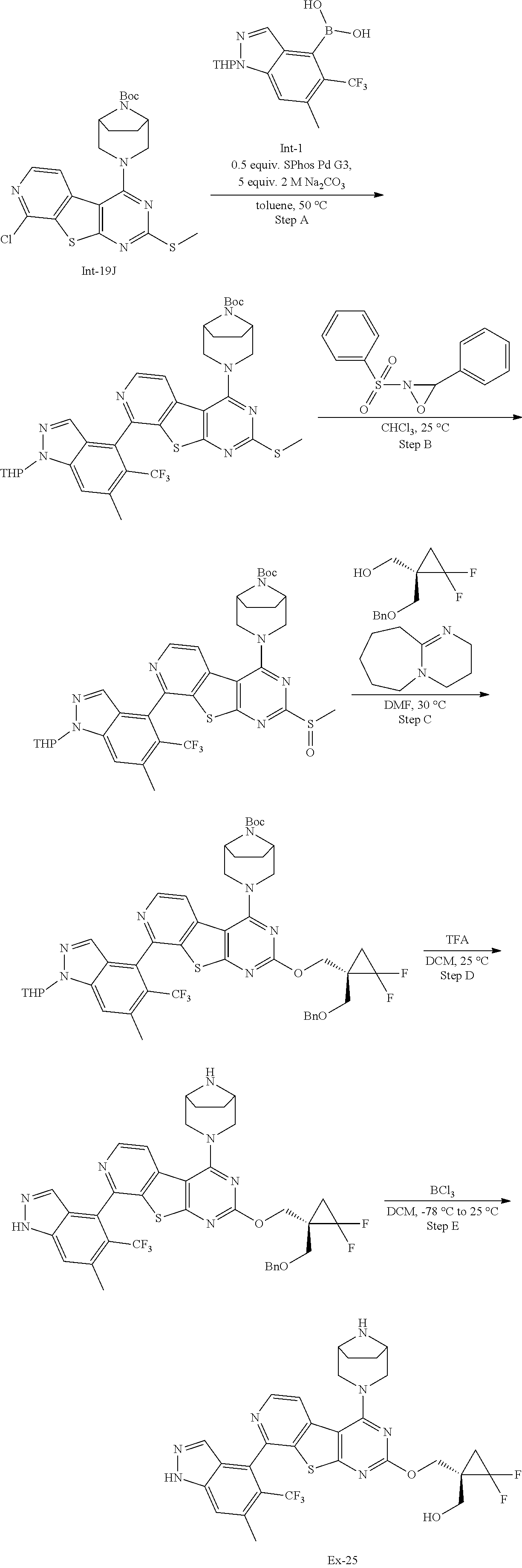

### Step A: Tert-butyl (1R,5S)-3-(8-(6-methyl-1-(tetrahydro-2H-pyran-2-yl)-5-(trifluoromethyl)-1H-indazol-4-yl)-2-(methylthio)pyrido[4′,3′:4,5]thieno[2,3-d]pyrimidin-4-yl)-3,8-diazabicyclo[3.2.1]octane-8-carboxylate

To a solution of tert-butyl (1R,5S)-3-(8-chloro-2-(methylthio)pyrido[4′,3′:4,5]thieno[2,3-d]pyrimidin-4-yl)-3,8-diazabicyclo[3.2.1]octane-8-carboxylate (Int-19J) (300 mg, 0.628 mmol) in toluene (4 mL) was added (6-methyl-1-(tetrahydro-2H-pyran-2-yl)-5-(trifluoromethyl)-1H-indazol-4-yl)boronic acid (Int-1) (309 mg, 0.941 mmol), SPhos Pd G3 (245 mg, 0.314 mmol), and Na₂CO₃ (1.57 mL, 3.14 mmol, 2 M in H₂O) at 20° C. in a glove box. The reaction mixture was heated to 50° C. and stirred for 1 h. The reaction solution was diluted with EtOAc (10 mL), filtered, and dried over Na₂SO₄. The mixture was filtered and the solvent was evaporated under reduced pressure. The crude product was purified by flash silica gel chromatography (0 to 20% EtOAc in petroleum ether) to give tert-butyl (1R,5S)-3-(8-(6-methyl-1-(tetrahydro-2H-pyran-2-yl)-5-(trifluoromethyl)-1H-indazol-4-yl)-2-(methylthio)pyrido[4′,3′:4,5]thieno[2,3-d]pyrimidin-4-yl)-3,8-diazabicyclo[3.2.1]octane-8-carboxylate. MS (ESI) [M+H]⁺: m/z 726.

### Step B: Tert-butyl (1R,5S)-3-(8-(6-methyl-1-(tetrahydro-2H-pyran-2-yl)-5-(trifluoromethyl)-1H-indazol-4-yl)-2-(methylsulfinyl)pyrido[4′,3′:4,5]thieno[2,3-d]pyrimidin-4-yl)-3,8-diazabicyclo[3.2.1]octane-8-carboxylate

Tert-butyl (1R,5S)-3-(8-(6-methyl-1-(tetrahydro-2H-pyran-2-yl)-5-(trifluoromethyl)-1H-indazol-4-yl)-2-(methylthio)pyrido[4′,3′:4,5]thieno[2,3-d]pyrimidin-4-yl)-3,8-diazabicyclo[3.2.1]octane-8-carboxylate (1.2 g, 1.653 mmol) was taken up in CHCl₃ (16.5 ml) and 3-phenyl-2-(phenylsulfonyl)-1,2-oxaziridine (0.48 g, 1.82 mmol) was added. The reaction was stirred at 25° C. The reaction was concentrated in vacuo and purified by silica gel column chromatography (0 to 100% (3:1 EtOAc:EtOH) in hex) to afford tert-butyl (1R,5S)-3-(8-(6-methyl-1-(tetrahydro-2H-pyran-2-yl)-5-(trifluoromethyl)-1H-indazol-4-yl)-2-(methylsulfinyl)pyrido[4′,3′:4,5]thieno[2,3-d]pyrimidin-4-yl)-3,8-diazabicyclo[3.2.1]octane-8-carboxylate. MS (ESI) [M+H]⁺: m/z 742.

### Step C: Tert-butyl (1R,5S)-3-(2-(((R)-1-((benzyloxy)methyl)-2,2-difluorocyclopropyl)methoxy)-8-(6-methyl-1-(tetrahydro-2H-pyran-2-yl)-5-(trifluoromethyl)-1H-indazol-4-yl)pyrido[4′,3′:4,5]thieno[2,3-d]pyrimidin-4-yl)-3,8-diazabicyclo[3.2.1]octane-8-carboxylate

(S)-(1-((benzyloxy)methyl)-2,2-difluorocyclopropyl)methanol (92 mg, 0.404 mmol) and tert-butyl (1R,5S)-3-(8-(6-methyl-1-(tetrahydro-2H-pyran-2-yl)-5-(trifluoromethyl)-1H-indazol-4-yl)-2-(methylsulfinyl)pyrido[4′,3′:4,5]thieno[2,3-d]pyrimidin-4-yl)-3,8-diazabicyclo[3.2.1]octane-8-carboxylate (60 mg, 0.081 mmol) were taken up in DMF (809 μL). DBU (61.0 μL, 0.404 mmol) was added and the reaction was stirred at 30° C. overnight. The reaction was extracted with EtOAc (3×), washed with brine, dried over Na₂SO₄, and concentrated under reduced pressure to afford tert-butyl (1R,5S)-3-(2-(((R)-1-((benzyloxy)methyl)-2,2-difluorocyclopropyl)methoxy)-8-(6-methyl-1-(tetrahydro-2H-pyran-2-yl)-5-(trifluoromethyl)-1H-indazol-4-yl)pyrido[4′,3′:4,5]thieno[2,3-d]pyrimidin-4-yl)-3,8-diazabicyclo[3.2.1]octane-8-carboxylate, which was used directly in the next step without further purification. MS (ESI) [M+H]⁺: m/z 906.

### Step D: 2-(((R)-1-((benzyloxy)methyl)-2,2-difluorocyclopropyl)methoxy)-4-((1R,5S)-3,8-diazabicyclo[3.2.1]octan-3-yl)-8-(6-methyl-5-(trifluoromethyl)-1H-indazol-4-yl)pyrido[4′,3′:4,5]thieno[2,3-d]pyrimidine

Tert-butyl (1R,5S)-3-(2-(((R)-1-((benzyloxy)methyl)-2,2-difluorocyclopropyl)methoxy)-8-(6-methyl-1-(tetrahydro-2H-pyran-2-yl)-5-(trifluoromethyl)-1H-indazol-4-yl)pyrido[4′,3′:4,5]thieno[2,3-d]pyrimidin-4-yl)-3,8-diazabicyclo[3.2.1]octane-8-carboxylate (73.4 mg, 0.081 mmol) was taken up in DCM (608 μL) and TFA (203 μL). The reaction was stirred at 25° C. for 4 hours. The reaction was quenched with NaHCO₃, extracted with DCM (3×), and dried over Na₂SO₄. The reaction was concentrated under reduced pressure, taken up in DMSO (1.5 mL), filtered, and purified by mass triggered reverse phase HPLC (MeCN/water with 0.1% NH₄OH modifier) to afford 2-(((R)-1-((benzyloxy)methyl)-2,2-difluorocyclopropyl)methoxy)-4-((1R,5S)-3,8-diazabicyclo[3.2.1]octan-3-yl)-8-(6-methyl-5-(trifluoromethyl)-1H-indazol-4-yl)pyrido[4′,3′:4,5]thieno[2,3-d]pyrimidine. MS (ESI) [M+H]⁺: m/z 722.

### Step E: ((1R)-1-(((4-((1R,5S)-3,8-diazabicyclo[3.2.1]octan-3-yl)-8-(6-methyl-5-(trifluoromethyl)-1H-indazol-4-yl)pyrido[4′,3′:4,5]thieno[2,3-d]pyrimidin-2-yl)oxy)methyl)-2,2-difluorocyclopropyl)methanol (Ex-25)

2-(((R)-1-((benzyloxy)methyl)-2,2-difluorocyclopropyl)methoxy)-4-((1R,5S)-3,8-diazabicyclo[3.2.1]octan-3-yl)-8-(6-methyl-5-(trifluoromethyl)-1H-indazol-4-yl)pyrido[4′,3′:4,5]thieno[2,3-d]pyrimidine (11.4 mg, 0.016 mmol) was taken up in DCM (158 μL) and cooled to −78° C. Boron trichloride (47.4 μL, 0.047 mmol, 1M in DCM) was added via syringe and the reaction was stirred at −78° C. for 1 hr. The reaction was warmed to 25° C. and stirred for another 1 hr. The reaction was quenched with MeOH (0.5 mL) and concentrated under reduced pressure. The residue was taken up in DMSO (1.0 mL), filtered, and purified by mass triggered reverse phase HPLC (MeCN/water with NH₄OH modifier) to afford ((1R)-1-(((4-((1R,5S)-3,8-diazabicyclo[3.2.1]octan-3-yl)-8-(6-methyl-5-(trifluoromethyl)-1H-indazol-4-yl)pyrido[4′,3′:4,5]thieno[2,3-d]pyrimidin-2-yl)oxy)methyl)-2,2-difluorocyclopropyl)methanol (Ex-25). MS (ESI) [M+H]⁺: m/z 632. ¹H NMR (600 MHz, DMSO-d₆) δ 8.69 (d, J=5.6 Hz, 1H), 7.76 (s, 1H), 7.72 (d, J=5.6 Hz, 1H), 7.49 (s, 1H), 5.07 (s, 1H), 4.55-4.38 (m, 2H), 4.16-4.00 (m, 2H), 3.66-3.45 (m, 7H), 2.67 (s, 3H), 1.66 (d, J=5.8 Hz, 2H), 1.60-1.49 (in, 4H).

The examples in the table below were synthesized using a similar procedure as described in the synthesis of Ex-25 by making the appropriate substitutions for starting material, intermediates, and/or reagents. Appropriate substitutions are available commercially, synthesized as described in the literature, synthesized using methods available to those skilled in the art, or synthesized as described herein.

| | Starting | | | [M + H]⁺ |
|---|---|---|---|---|
| Ex. | materials | Structure | Compound Name | Found |
| Ex- 26 | 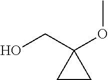 (1- methoxycyclopro- pyl)methanol | 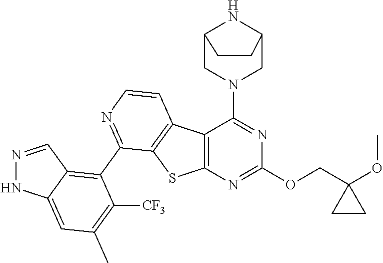 [SMILES: COC1(COc2nc(N3CC4CCC(C3)N4)c3c(n2)sc2c(-c4c(C(F)(F)F)c(C)cc5[nH]ncc45)nccc23)CC1] | 4-((1R,5S)-3,8- diazabicyclo[3.2.1]octan-3-yl)-2- ((1- methoxycyclopropyl)methoxy)-8- (6-methyl-5-(trifluoromethyl)- 1H-indazol-4- yl)pyrido[4′,3′:4,5]thieno[2,3- d]pyrimidine | 596 |
| Ex- 27 | 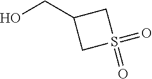 (hydroxymethyl)thi- etane 1,1- dioxide | 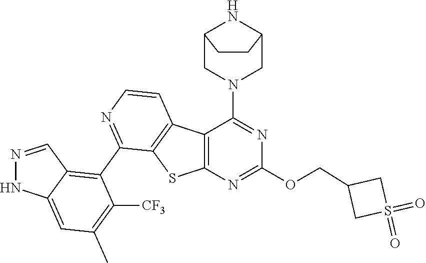 [SMILES: Cc1cc2[nH]ncc2c(-c2nccc3c2sc2nc(OCC4CS(=O)(=O)C4)nc(N4CC5CCC(C4)N5)c23)c1C(F)(F)F] | 3-(((4-((1R,5S)-3,8- diazabicyclo[3.2.1]octan-3-yl)-8- (6-methy1-5-(trifluoromethyl)- 1H-indazol-4- yl)pyrido[4′,3′:4,5]thieno[2,3- d]pyrimidin-2- yl)oxy)methyl)thietane 1,1- dioxide | 630 |
| Ex- 28 | 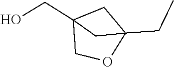 (1- (fluoromethyl)-2- oxabicyclo[2.1.1] hexan-4- yl)methanol | 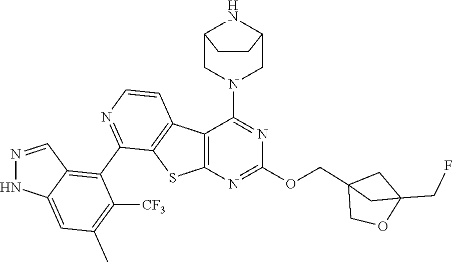 [SMILES: Cc1cc2[nH]ncc2c(-c2nccc3c2sc2nc(OCC45COC(CF)(C4)C5)nc(N4CC5CCC(C4)N5)c23)c1C(F)(F)F] | 4-((1R,5S)-3,8- diazabicyclo[3.2.1]octan-3-yl)-2- ((1-(fluoromethyl)-2- oxabicyclo[2.1.1]hexan-4- yl)methoxy)-8-(6-methy1-5- (trifluoromethyl)-1H-indazol-4- yl)pyrido[4′,3′:4,5]thieno[2,3- d]pyrimidine | 640 |
| Ex- 29 | 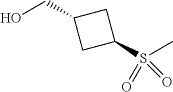 ((1r,3r)-3- (methylsulfonyl) cyclobutyl)metha- nol | 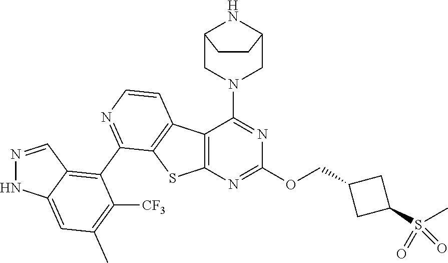 [SMILES: Cc1cc2[nH]ncc2c(-c2nccc3c2sc2nc(OC[C@H]4C[C@H](S(C)(=O)=O)C4)nc(N4CC5CCC(C4)N5)c23)c1C(F)(F)F] | 4-((1R,5S)-3,8- diazabicyclo[3.2.1]octan-3-yl)-8- (6-methyl-5-(trifluoromethyl)- 1H-indazol-4-yl)-2-(((1r,3R)-3- (methylsulfonyl)cyclobutyl)meth- oxy)pyrido[4′,3′:4,5] thieno[2,3- d]pyrimidine | 658 |
| Ex- 30 | 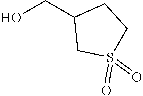 3- (hydroxymethyl)tetra- hydrothiophene 1,1-dioxide | 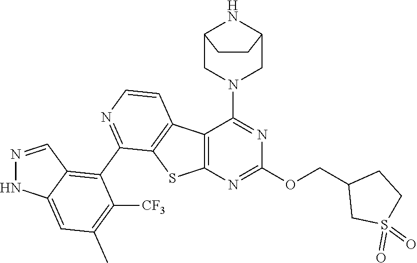 [SMILES: Cc1cc2[nH]ncc2c(-c2nccc3c2sc2nc(OCC4CCS(=O)(=O)C4)nc(N4CC5CCC(C4)N5)c23)c1C(F)(F)F] | 3-(((4-((1R,5S)-3,8- diazabicyclo[3.2.1]octan-3-yl)-8- (6-methyl-5-(trifluoromethyl)- 1H-indazol-4- yl)pyrido[4′,3′:4,5]thieno[2,3- d]pyrimidin-2- yl)oxy)methyl)tetrahydrothio- phene 1,1-dioxide | 644 |
| Ex- 31 | 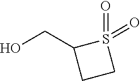 (hydroxymethyl)thi- etane 1,1- dioxide | 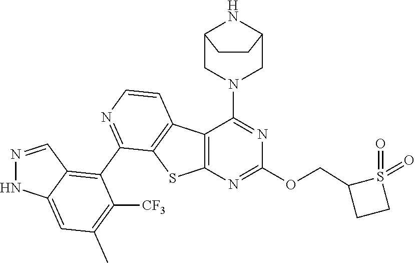 [SMILES: Cc1cc2[nH]ncc2c(-c2nccc3c2sc2nc(OCC4CCS4(=O)=O)nc(N4CC5CCC(C4)N5)c23)c1C(F)(F)F] | 2-(((4-((1R,5S)-3,8- diazabicyclo[3.2.1]octan-3-yl)-8- (6-methyl-5-(trifluoromethyl)- 1H-indazol-4- yl)pyrido[4′,3′:4,5]thieno[2,3- d]pyrimidin-2- yl)oxy)methyl)thietane 1,1- dioxide | 630 |
| Ex- 32 | 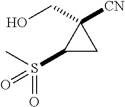 (1R,2R)-1- (hydroxymethyl)- 2- (methylsulfonyl) cyclopropane-1- carbonitrile | 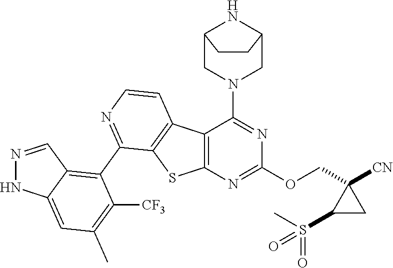 [SMILES: Cc1cc2[nH]ncc2c(-c2nccc3c2sc2nc(OC[C@]4(C#N)C[C@H]4S(C)(=O)=O)nc(N4CC5CCC(C4)N5)c23)c1C(F)(F)F] | (1R,2R)-1-(((4-((1R,5S)-3,8- diazabicyclo[3.2.1]octan-3-yl)-8- (6-methyl-5-(trifluoromethyl)- 1H-indazol-4- yl)pyrido[4′,3′:4,5]thieno[2,3- d]pyrimidin-2-yl)oxy)methyl)-2- (methylsulfonyl)cyclopropane-1- carbonitrile | 669 |
| Ex- 33 | 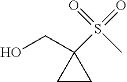 (1- (methylsulfonyl) cyclopropyl)meth- anol | 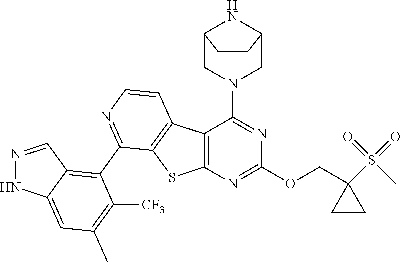 [SMILES: Cc1cc2[nH]ncc2c(-c2nccc3c2sc2nc(OCC4(S(C)(=O)=O)CC4)nc(N4CC5CCC(C4)N5)c23)c1C(F)(F)F] | 4-((1R,5S)-3,8- diazabicyclo[3.2.1]octan-3-yl)-8- (6-methyl-5-(trifluoromethyl)- 1H-indazol-4-yl)-2-((1- (methylsulfonyl)cyclopropyl)meth- oxy)pyrido[4′,3′:4,5]thieno[2,3- d]pyrimidine | 644 |
| Ex- 34 | 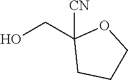 2- (hydroxymethyl)tetra- hydrofuran-2- carbonitrile | 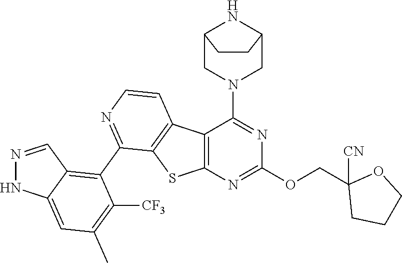 [SMILES: Cc1cc2[nH]ncc2c(-c2nccc3c2sc2nc(OCC4(C#N)CCCO4)nc(N4CC5CCC(C4)N5)c23)c1C(F)(F)F] | 2-(((4-((1R,5S)-3,8- diazabicyclo[3.2.1]octan-3-yl)-8- (6-methyl-5-(trifluoromethyl)- 1H-indazol-4- yl)pyrido[4′,3′:4,5]thieno[2,3- d]pyrimidin-2- yl)oxy)methyl)tetrahydrofuran-2- carbonitrile | 621 |
| Ex- 35 | 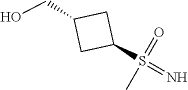 (S)-((1r,3S)-3- (hydroxymethyl) cyclobutyl)(imino) (methyl)-λ⁶- sulfanone | 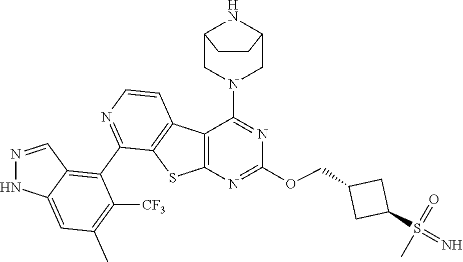 [SMILES: Cc1cc2[nH]ncc2c(-c2nccc3c2sc2nc(OC[C@H]4C[C@H](S(C)(=N)=O)C4)nc(N4CC5CCC(C4)N5)c23)c1C(F)(F)F] | (1S)-((1R,3S)-3-(((4-((1R,5S)- 3,8-diazabicyclo[3.2.1]octan-3- yl)-8-(6-methyl-5- (trifluoromethyl)-1H-indazol-4- yl)pyrido[4′,3′:4,5]thieno[2,3- d]pyrimidin-2- yl)oxy)methyl)cyclobutyl)(imino) (methyl)-λ⁶-sulfanone | 657 |
| Ex- 36 | 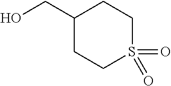 (hydroxymethyl)tetra- hydro-2H- thiopyran 1,1- dioxide | 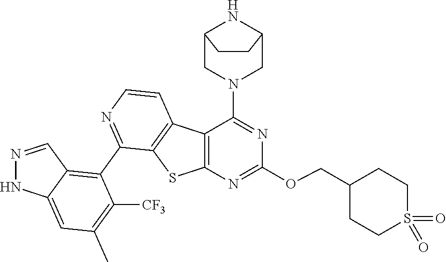 [SMILES: Cc1cc2[nH]ncc2c(-c2nccc3c2sc2nc(OCC4CCS(=O)(=O)CC4)nc(N4CC5CCC(C4)N5)c23)c1C(F)(F)F] | 4-(((4-((1R,5S)-3,8- diazabicyclo[3.2.1]octan-3-yl)-8- (6-methy1-5-(trifluoromethyl)- 1H-indazol-4- yl)pyrido[4′,3′:4,5]thieno[2,3- d]pyrimidin-2- yl)oxy)methyl)tetrahydro-2H- thiopyran 1,1-dioxide | 658 |
| Ex- 37 | 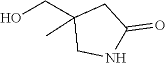 4- (hydroxymethyl)- 4- methylpyrrolidin- 2-one | 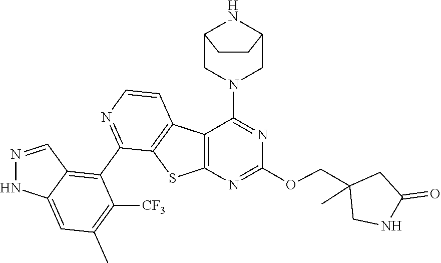 [SMILES: Cc1cc2[nH]ncc2c(-c2nccc3c2sc2nc(OCC4(C)CNC(=O)C4)nc(N4CC5CCC(C4)N5)c23)c1C(F)(F)F] | 4-(((4-((1R,5S)-3,8- diazabicyclo[3.2.1]octan-3-yl)-8- (6-methy1-5-(trifluoromethyl)- 1H-indazol-4- yl)pyrido[4′,3′:4,5]thieno[2,3- d]pyrimidin-2-yl)oxy)methyl)-4- methylpyrrolidin-2-one | 623 |
| Ex- 38 | 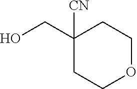 4- (hydroxymethyl)tetra- hydro-2H- pyran-4- carbonitrile | 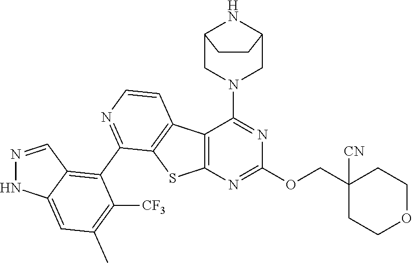 [SMILES: Cc1cc2[nH]ncc2c(-c2nccc3c2sc2nc(OCC4(C#N)CCOCC4)nc(N4CC5CCC(C4)N5)c23)c1C(F)(F)F] | 4-(((4-((1R,5S)-3,8- diazabicyclo[3.2.1]octan-3-yl)-8- (6-methyl-5-(trifluoromethyl)- 1H-indazol-4- yl)pyrido[4′,3′:4,5]thieno[2,3- d]pyrimidin-2- yl)oxy)methyl)tetrahydro-2H- pyran-4-carbonitrile | 635 |
| Ex- 39 | 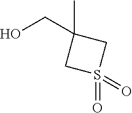 3- (hydroxymethyl)- 3-methylthietane 1,1-dioxide | 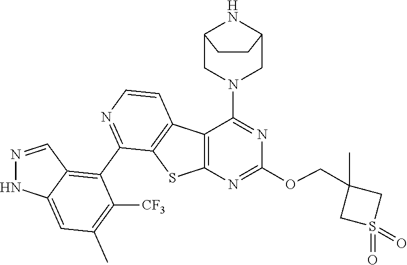 [SMILES: Cc1cc2[nH]ncc2c(-c2nccc3c2sc2nc(OCC4(C)CS(=O)(=O)C4)nc(N4CC5CCC(C4)N5)c23)c1C(F)(F)F] | 3-(((4-((1R,5S)-3,8- diazabicyclo[3.2.1]octan-3-yl)-8- (6-methyl-5-(trifluoromethyl)- 1H-indazol-4- yl)pyrido[4′,3′:4,5]thieno[2,3- d]pyrimidin-2-yl)oxy)methyl)-3- methylthietane 1,1-dioxide | 644 |
| Ex- 40 | 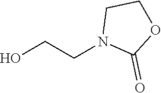 3-(2- hydroxyethyl)oxa- zolidin-2-one | 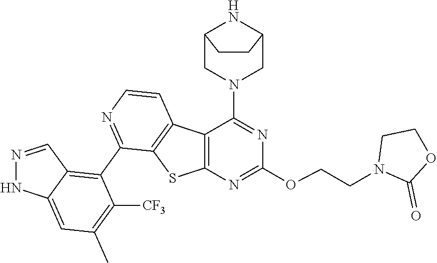 [SMILES: Cc1cc2[nH]ncc2c(-c2nccc3c2sc2nc(OCCN4CCOC4=O)nc(N4CC5CCC(C4)N5)c23)c1C(F)(F)F] | 3-(2-((4-((1R,5S)-3,8- diazabicyclo[3.2.1]octan-3-yl)-8- (6-methyl-5-(trifluoromethyl)- 1H-indazol-4- yl)pyrido[4′,3′:4,5]thieno[2,3- d]pyrimidin-2- yl)oxy)ethyl)oxazolidin-2-one | 625 |
| Ex- 41 | 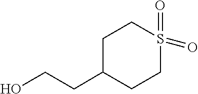 [SMILES: O=S1(=O)CCC(CCO)CC1] 4-(2- hydroxyethyl)tetra- hydro-2H- thiopyran 1,1- dioxide | 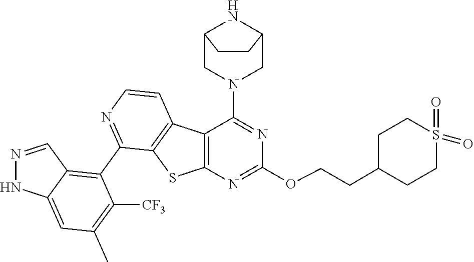 [SMILES: Cc1cc2[nH]ncc2c(-c2nccc3c2sc2nc(OCCC4CCS(=O)(=O)CC4)nc(N4CC5CCC(C4)N5)c23)c1C(F)(F)F] | 4-(2-((4-((1R,5S)-3,8- diazabicyclo[3.2.1]octan-3-yl)-8- (6-methyl-5-(trifluoromethyl)- 1H-indazol-4- yl)pyrido[4′,3′:4,5]thieno[2,3- d]pyrimidin-2- yl)oxy)ethyl)tetrahydro-2H- thiopyran 1,1-dioxide | 672 |
| Ex- 42 | 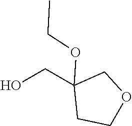 (3- ethoxytetrahydro furan-3- yl)methanol | 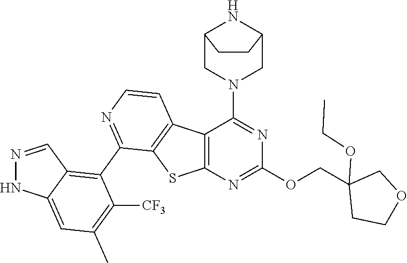 [SMILES: CCOC1(COc2nc(N3CC4CCC(C3)N4)c3c(n2)sc2c(-c4c(C(F)(F)F)c(C)cc5[nH]ncc45)nccc23)CCOC1] | 4-((1R,5S)-3,8- diazabicyclo[3.2.1]octan-3-yl)-2- ((3-ethoxytetrahydrofuran-3- yl)methoxy)-8-(6-methyl-5- (trifluoromethyl)-1H-indazol-4- yl)pyrido[4′,3′:4,5]thieno[2,3- d]pyrimidine | 640 |
| Ex- 43 | 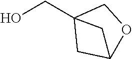 (2- oxabicyclo[2.1.1] hexan-4- yl)methanol | 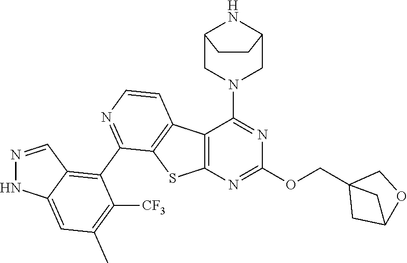 [SMILES: Cc1cc2[nH]ncc2c(-c2nccc3c2sc2nc(OCC45COC(C4)C5)nc(N4CC5CCC(C4)N5)c23)c1C(F)(F)F] | 2-((2-oxabicyclo[2.1.1]hexan-4- yl)methoxy)-4-((1R,5S)-3,8- diazabicyclo[3.2.1]octan-3-yl)-8- (6-methyl-5-(trifluoromethyl)- 1H-indazol-4- yl)pyrido[4′,3′:4,5]thieno[2,3- d]pyrimidine | 608 |
| Ex- 44 | 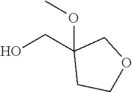 (3- methoxytetrahydro- furan-3- yl)methanol | 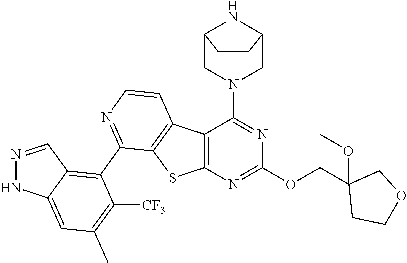 [SMILES: COC1(COc2nc(N3CC4CCC(C3)N4)c3c(n2)sc2c(-c4c(C(F)(F)F)c(C)cc5[nH]ncc45)nccc23)CCOC1] | 4-((1R,5S)-3,8- diazabicyclo[3.2.1]octan-3-yl)-2- ((3-methoxytetrahydrofuran-3- yl)methoxy)-8-(6-methyl-5- (trifluoromethyl)-1H-indazol-4- yl)pyrido[4′,3′:4,5]thieno[2,3- d]pyrimidine | 626 |
| Ex- 45 | 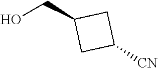 (1r,3r)-3- (hydroxymethyl) cyclobutane-1- carbonitrile | 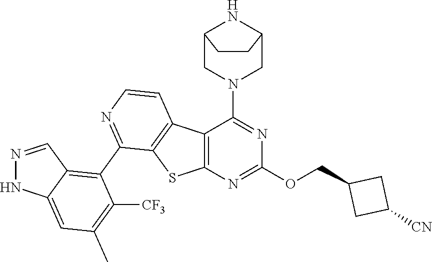 [SMILES: Cc1cc2[nH]ncc2c(-c2nccc3c2sc2nc(OC[C@H]4C[C@H](C#N)C4)nc(N4CC5CCC(C4)N5)c23)c1C(F)(F)F] | (1R,3r)-3-(((4-((1R,5S)-3,8- diazabicyclo[3.2.1]octan-3-yl)-8- (6-methyl-5-(trifluoromethyl)- 1H-indazol-4- yl)pyrido[4′,3′:4,5]thieno[2,3- d]pyrimidin-2- yl)oxy)methyl)cyclobutane-1- carbonitrile | 605 |
| Ex- 46 | 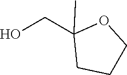 (2- methyltetrahydro- furan-2- yl)methanol | 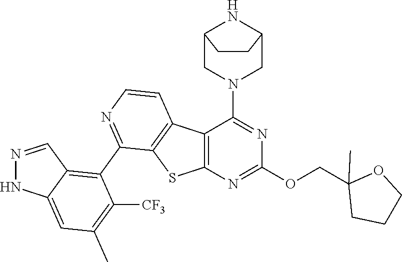 [SMILES: Cc1cc2[nH]ncc2c(-c2nccc3c2sc2nc(OCC4(C)CCCO4)nc(N4CC5CCC(C4)N5)c23)c1C(F)(F)F] | 4-((1R,5S)-3,8- diazabicyclo[3.2.1]octan-3-yl)-8- (6-methy1-5-(trifluoromethyl)- 1H-indazol-4-yl)-2-((2- methyltetrahydrofuran-2- yl)methoxy)pyrido[4′,3′:4,5]thi- eno[2,3-d]pyrimidine | 610 |
| Ex- 47 | 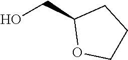 (R)- (tetrahydrofuran- 2-yl)methanol | 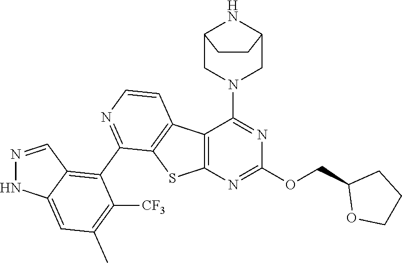 [SMILES: Cc1cc2[nH]ncc2c(-c2nccc3c2sc2nc(OC[C@H]4CCCO4)nc(N4CC5CCC(C4)N5)c23)c1C(F)(F)F] | 4-((1R,5S)-3,8- diazabicyclo[3.2.1]octan-3-yl)-8- (6-methyl-5-(trifluoromethyl)- 1H-indazol-4-yl)-2-(((R)- tetrahydrofuran-2- yl)methoxy)pyrido[4′,3′:4,5]thi- eno[2,3-d]pyrimidine | 596 |
| Ex- 48 | 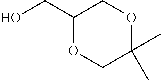 (5,5-dimethyl- 1,4-dioxan-2- yl)methanol | 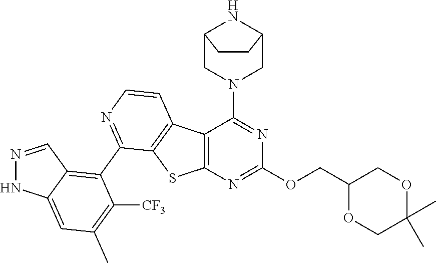 [SMILES: Cc1cc2[nH]ncc2c(-c2nccc3c2sc2nc(OCC4COC(C)(C)CO4)nc(N4CC5CCC(C4)N5)c23)c1C(F)(F)F] | 4-((1R,5S)-3,8- diazabicyclo[3.2.1]octan-3-yl)-2- ((5,5-dimethy1-1,4-dioxan-2- yl)methoxy)-8-(6-methy1-5- (trifluoromethyl)-1H-indazol-4- yl)pyrido[4′,3′:4,5]thieno[2,3- d]pyrimidine | 640 |
| Ex- 49 | 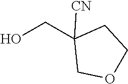 3- (hydroxymethyl)tetra- hydrofuran-3- carbonitrile | 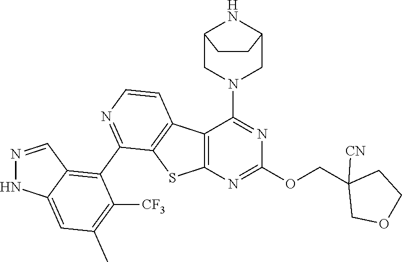 [SMILES: Cc1cc2[nH]ncc2c(-c2nccc3c2sc2nc(OCC4(C#N)CCOC4)nc(N4CC5CCC(C4)N5)c23)c1C(F)(F)F] | 3-(((4-((1R,5S)-3,8- diazabicyclo[3.2.1]octan-3-yl)-8- (6-methyl-5-(trifluoromethyl)- 1H-indazol-4- yl)pyrido[4′,3′:4,5]thieno[2,3- d]pyrimidin-2- yl)oxy)methyl)tetrahydrofuran-3- carbonitrile | 621 |
| Ex- 50 | 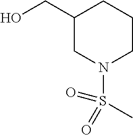 (1- (methylsulfonyl) piperidin-3- yl)methanol | 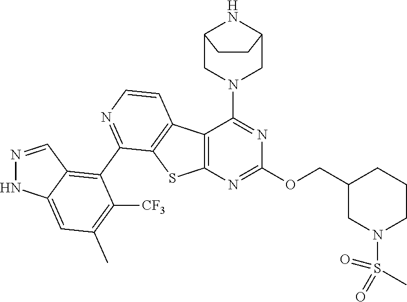 [SMILES: Cc1cc2[nH]ncc2c(-c2nccc3c2sc2nc(OCC4CCCN(S(C)(=O)=O)C4)nc(N4CC5CCC(C4)N5)c23)c1C(F)(F)F] | 4-((1R,5S)-3,8- diazabicyclo[3.2.1]octan-3-yl)-8- (6-methyl-5-(trifluoromethyl)- 1H-indazol-4-yl)-2-((1- (methylsulfonyl)piperidin-3- yl)methoxy)pyrido[4′,3′:4,5]thi- eno[2,3-d]pyrimidine | 687 |

### Example 51: ((1R)-1-(((4-((1R,5S)-3,8-diazabicyclo[3.2.1]octan-3-yl)-8-(5-(difluoromethoxy)-6-methyl-1H-indazol-4-yl)pyrido[4′,3′:4,5]thieno[2,3-d]pyrimidin-2-yl)oxy)methyl)-2,2-difluorocyclopropyl)methanol (Ex-51)

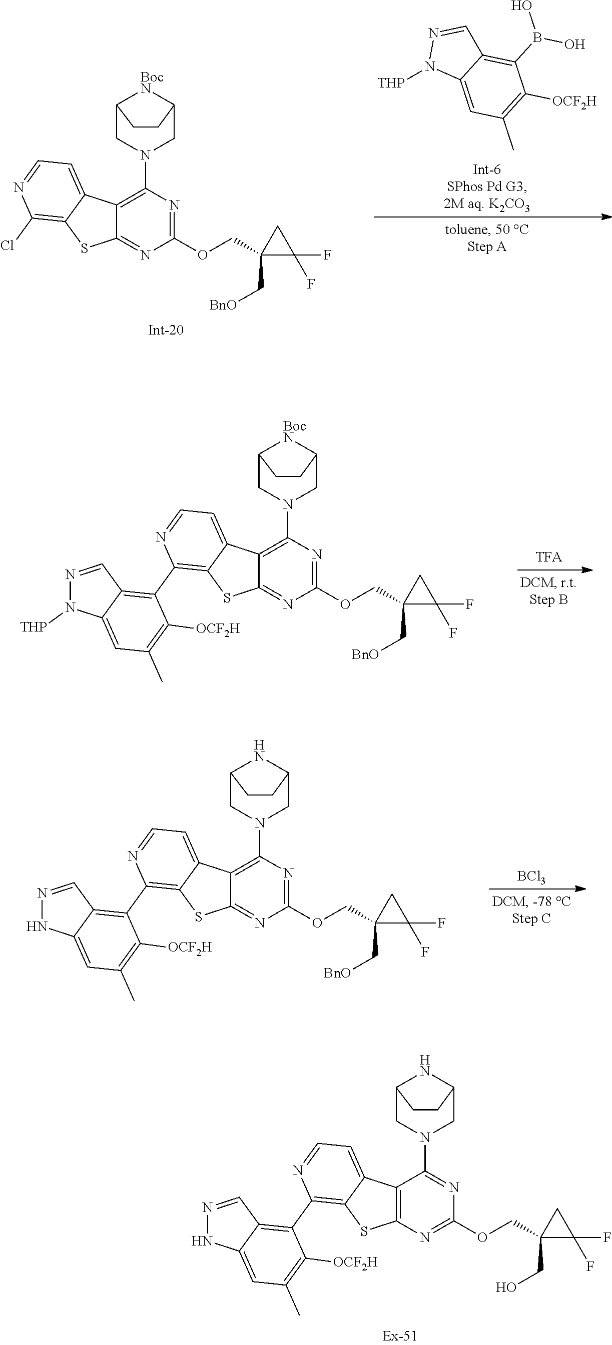

### Step A: tert-butyl (1R,5S)-3-(2-(((R)-1-((benzyloxy)methyl)-2,2-difluorocyclopropyl)methoxy)-8-(5-(difluoromethoxy)-6-methyl-1-(tetrahydro-2H-pyran-2-yl)-1H-indazol-4-yl)pyrido[4′,3′:4,5]thieno[2,3-d]pyrimidin-4-yl)-3,8-diazabicyclo[3.2.1]octane-8-carboxylate

To a solution of tert-butyl (1R,5S)-3-(2-(((R)-1-((benzyloxy)methyl)-2,2-difluorocyclopropyl)methoxy)-8-chloropyrido[4′,3′:4,5]thieno[2,3-d]pyrimidin-4-yl)-3,8-diazabicyclo[3.2.1]octane-8-carboxylate (Int-20) (2.00 g, 3.04 mmol) in toluene (20 mL) was added (5-(difluoromethoxy)-6-methyl-1-(tetrahydro-2H-pyran-2-yl)-1H-indazol-4-yl)boronic acid (Int-6) (1.49 g, 4.56 mmol), SPhos Pd G3 (1.19 g, 1.52 mmol), and potassium carbonate (4.56 mL, 9.12 mmol, 2 M in H₂O) at 25° C. The reaction was placed under N₂ atmosphere and stirred at 50° C. for 16 h. The reaction mixture was cooled to room temperature and diluted with EtOAc (20 mL). The reaction mixture was washed with brine (20 mL), dried over Na₂SO₄, filtered, and concentrated in vacuo. The residue was purified by flash silica gel chromatography (0 to 50% EtOAc in petroleum ether) to give tert-butyl (1R,5S)-3-(2-(((R)-1-((benzyloxy)methyl)-2,2-difluorocyclopropyl)methoxy)-8-(5-(difluoromethoxy)-6-methyl-1-(tetrahydro-2H-pyran-2-yl)-1H-indazol-4-yl)pyrido[4′,3′:4,5]thieno[2,3-d]pyrimidin-4-yl)-3,8-diazabicyclo[3.2.1]octane-8-carboxylate. MS (ESI) [M+H]⁺: m/z 904.

### Step B: 2-(((R)-1-((benzyloxy)methyl)-2,2-difluorocyclopropyl)methoxy)-4-((1R,5S)-3,8-diazabicyclo[3.2.1]octan-3-yl)-8-(5-(difluoromethoxy)-6-methyl-1H-indazol-4-yl)pyrido[4′,3′:4,5]thieno[2,3-d]pyrimidine

To a solution of tert-butyl (1R,5S)-3-(2-(((R)-1-((benzyloxy)methyl)-2,2-difluorocyclopropyl)methoxy)-8-(5-(difluoromethoxy)-6-methyl-1-(tetrahydro-2H-pyran-2-yl)-1H-indazol-4-yl)pyrido[4′,3′:4,5]thieno[2,3-d]pyrimidin-4-yl)-3,8-diazabicyclo[3.2.1]octane-8-carboxylate (240 mg, 0.264 mmol) in DCM (1 mL) was added TFA (0.35 mL, 4.54 mmol). The mixture was stirred at room temperature for 1 h. The reaction mixture was stirred at 30° C. for 1 h. The mixture was evaporated under reduced pressure to give crude 2-(((R)-1-((benzyloxy)methyl)-2,2-difluorocyclopropyl)methoxy)-4-((1R,5S)-3,8-diazabicyclo[3.2.1]octan-3-yl)-8-(5-(difluoromethoxy)-6-methyl-1H-indazol-4-yl)pyrido[4′,3′:4,5]thieno[2,3-d]pyrimidine, which was used directly in the next step without further purification. MS (ESI) [M+H]⁺: m/z 720.

### Step C: ((1R)-1-(((4-((1R,5S)-3,8-diazabicyclo[3.2.1]octan-3-yl)-8-(5-(difluoromethoxy)-6-methyl-1H-indazol-4-yl)pyrido[4′,3′:4,5]thieno[2,3-d]pyrimidin-2-yl)oxy)methyl)-2,2-difluorocyclopropyl)methanol (Ex-51)

To a stirred solution of 2-(((R)-1-((benzyloxy)methyl)-2,2-difluorocyclopropyl)methoxy)-4-((1R,5S)-3,8-diazabicyclo[3.2.1]octan-3-yl)-8-(5-(difluoromethoxy)-6-methyl-1H-indazol-4-yl)pyrido[4′,3′:4,5]thieno[2,3-d]pyrimidine (190 mg, 0.264 mmol) in DCM (3 mL) was added boron trichloride (1.58 mL, 1.58 mmol, 1 M in DCM) at −78° C. The mixture was stirred at −78° C. for 1 h. The reaction mixture was quenched by the addition of MeOH (3 mL), warmed to room temperature, and concentrated in vacuo. The residue was dissolved in DMSO (1.5 mL) and MeCN (2.5 mL). The mixture was basified to pH 8 by 37% aq. NH₄OH and filtered. The filtrate was purified by preparative HPLC (MeCN/H₂O w/NH₄OH+NH₄HCO₃ modifier) to give ((1R)-1-(((4-((1R,5S)-3,8-diazabicyclo[3.2.1]octan-3-yl)-8-(5-(difluoromethoxy)-6-methyl-1H-indazol-4-yl)pyrido[4′,3′:4,5]thieno[2,3-d]pyrimidin-2-yl)oxy)methyl)-2,2-difluorocyclopropyl)methanol (Ex-51). MS (ESI) [M+H]⁺: m/z 630. ¹H NMR (400 MHz, MeOD) δ 8.79 (d, J=5.6 Hz, 1H), 7.86 (d, J=5.6 Hz, 1H), 7.69 (d, J=6.9 Hz, 2H), 6.72-6.24 (m, 1H), 4.72-4.57 (m, 2H), 4.44 (br t, J=15.9 Hz, 2H), 3.97 (br s, 2H), 3.90-3.75 (m, 4H), 2.58 (s, 3H), 2.09-1.92 (m, 4H), 1.66-1.46 (m, 2H).

The example in the table below was synthesized using a similar procedure as described in the synthesis of Ex-51 by making the appropriate substitutions for starting material, intermediates, and/or reagents. Appropriate substitutions are available commercially, synthesized as described in the literature, synthesized using methods available to those skilled in the art, or synthesized as described herein.

| | | | | [M + |
|---|---|---|---|---|
| | | | | H]⁺ |
| Ex. | Starting material | Structure | Compound Name | Found |
| Ex- 52 | 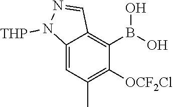 Int-7 | 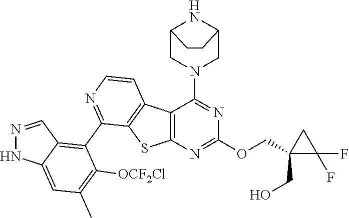 | ((1R)-1-(((4-((1R,5S)-3,8- diazabicyclo[3.2.1]octan-3- yl)-8-(5- (chlorodifluoromethoxy)-6- methyl-1H-indazol-4-yl) pyrido[4′,3′:4,5]thieno[2,3- d]pyrimidin-2- yl)oxy)methyl)-2,2- difluorocyclopro- pyl)methanol | 664 |
| Ex- 53 | 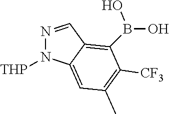 Int-1 | 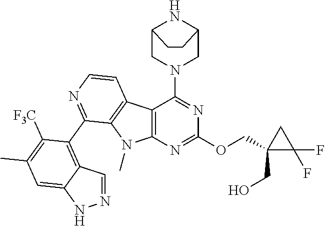 [SMILES: Cc1cc2[nH]ncc2c(-c2nccc3c4c(N5CC6CCC(C5)N6)nc(OC[C@]5(CO)CC5(F)F)nc4n(C)c23)c1C(F)(F)F] | ((1R)-1-(((4-((1R,5S)-3,8- diazabicyclo[3.2.1]octan-3- yl)-9-methyl-8-(6-methyl-5- (trifluoromethyl)-1H- indazol-4-yl)-9H- pyrido[4′,3′:4,5]pyrrolo[2,3- d]pyrimidin-2- yl)oxy)methyl)-2,2- difluorocyclopro- pyl)methanol | 629 |

### Example 54: 4-((1R,5S)-3,8-diazabicyclo[3.2.1]octan-3-yl)-8-(5-ethynyl-1H-thieno[3,2-f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidine (Ex-54)

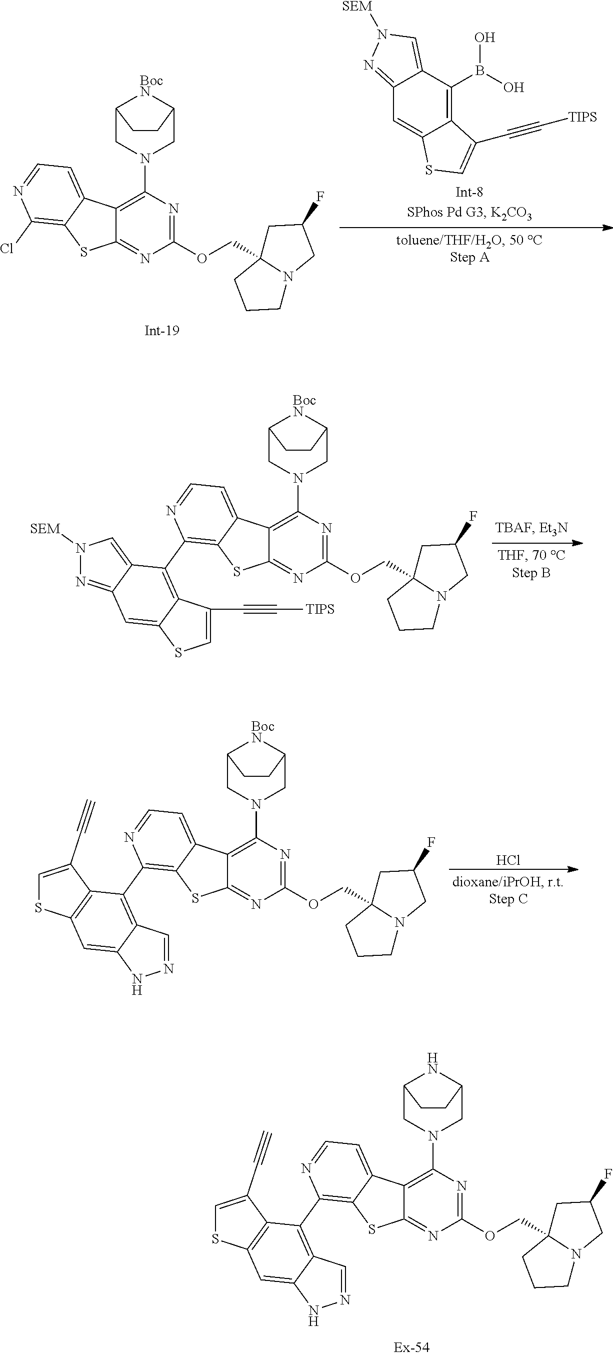

### Step A: tert-butyl (1R,5S)-3-(2-(((2R,7aS)-2-fluorotetrahydro-1H-pyrrolizin-7a(5H)-yl)methoxy)-8-(5-((triisopropylsilyl)ethynyl)-2-((2-(trimethylsilyl)ethoxy)methyl)-2H-thieno[3,2-f]indazol-4-yl)pyrido[4′,3′:4,5]thieno[2,3-d]pyrimidin-4-yl)-3,8-diazabicyclo[3.2.1]octane-8-carboxylate

To a solution of tert-butyl (1R,5S)-3-(8-chloro-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate (Int-19) (146 mg, 0.248 mmol) in dry toluene (1.5 mL) was added (5-((triisopropylsilyl)ethynyl)-2-((2-(trimethylsilyl)ethoxy)methyl)-2H-thieno[3,2-f]indazol-4-yl)boronic acid (Int-8) (146 mg, 0.276 mmol), SPhos Pd G3 (97 mg, 0.124 mmol), K₂CO₃ (0.372 mL, 0.743 mmol, 2M in H₂O) and THF (0.5 mL) at 25° C. The mixture was warmed to 50° C. and stirred for 1.5 h under N₂ atmosphere. The reaction mixture was cooled to room temperature and extracted with EtOAc (3×3 mL). The organic layer was dried over Na₂SO₄, filtered, and the filtrate was evaporated under reduced pressure. The residue was purified by preparative TLC (SiO₂, DCM:MeOH=15:1) to give tert-butyl (1R,5S)-3-(2-(((2R,7aS)-2-fluorotetrahydro-1H-pyrrolizin-7a(5H)-yl)methoxy)-8-(5-((triisopropylsilyl)ethynyl)-2-((2-(trimethylsilyl)ethoxy)methyl)-2H-thieno[3,2-f]indazol-4-yl)pyrido[4′,3′:4,5]thieno[2,3-d]pyrimidin-4-yl)-3,8-diazabicyclo[3.2.1]octane-8-carboxylate. MS (ESI) [M/2+H]⁺: m/z 519.

### Step B: tert-butyl (1R,5S)-3-(8-(5-ethynyl-1H-thieno[3,2-f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate

To a solution of tert-butyl (1R,5S)-3-(2-(((2R,7aS)-2-fluorotetrahydro-1H-pyrrolizin-7a(5H)-yl)methoxy)-8-(5-((triisopropylsilyl)ethynyl)-2-((2-(trimethylsilyl)ethoxy)methyl)-2H-thieno[3,2-J]indazol-4-yl)pyrido[4′,3′:4,5]thieno[2,3-d]pyrimidin-4-yl)-3,8-diazabicyclo[3.2.1]octane-8-carboxylate (34 mg, 0.033 mmol) in THF (0.1 mL) was added TBAF (0.131 mL, 0.131 mmol, 1 M in THF) and triethylamine (2.28 μL, 0.016 mmol) at 25° C. under N₂ atmosphere. The solution was warmed to 70° C. and stirred for 16 h. The mixture was cooled to room temperature, diluted with water (1 mL), and extracted with EtOAc (3×5 mL). The organic layer was dried over Na₂SO₄, filtered, and the filtrate was concentrated under reduced pressure. The residue was purified by preparative TLC (SiO₂, DCM:MeOH=10:1) to give tert-butyl (1R,5S)-3-(8-(5-ethynyl-1H-thieno[3,2-J]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate. MS (ESI) [M+H]⁺: m/z 751.

### Step C: 4-((1R,5S)-3,8-diazabicyclo[3.2.1]octan-3-yl)-8-(5-ethynyl-1H-thieno[3,2-f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidine (Ex-54)

To a solution of tert-butyl (1R,5S)-3-(8-(5-ethynyl-1H-thieno[3,2-J]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate (25 mg, 0.033 mmol) in i-PrOH (0.3 mL) was added HCl (0.6 mL, 2.400 mmol, 4M in dioxane) at 25° C. The mixture was stirred at 25° C. for 1 h. The mixture was evaporated under reduced pressure. The residue was purified by reverse preparative HPLC (MeCN/H₂O w/TFA modifier) to give 4-((1R,5S)-3,8-diazabicyclo[3.2.1]octan-3-yl)-8-(5-ethynyl-1H-thieno[3,2-J]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidine (Ex-54) as a TFA salt. MS (ESI) [M+H]⁺: m/z 651. ¹H NMR (400 MHz, MeOD) δ 8.81 (d, J=5.8 Hz, 1H), 8.37 (d, J=0.7 Hz, 1H), 8.02 (d, J=5.8 Hz, 1H), 7.90 (s, 1H), 7.85 (s, 1H), 5.44-5.69 (m, 1H), 4.66 (s, 3H), 4.33-4.46 (m, 1H), 4.18-4.29 (m, 2H), 4.12 (br dd, J=13.9, 3.8 Hz, 1H), 3.82-3.98 (m, 4H), 3.45 (td, J=10.7, 5.6 Hz, 1H), 2.90 (d, J=3.9 Hz, 1H), 2.52-2.77 (m, 2H), 2.29-2.47 (m, 3H), 1.93-2.22 (m, 5H).

### Example 55: 4-((1R,5S)-3,8-diazabicyclo[3.2.1]octan-3-yl)-8-(5-ethynyl-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidine (Ex-55)

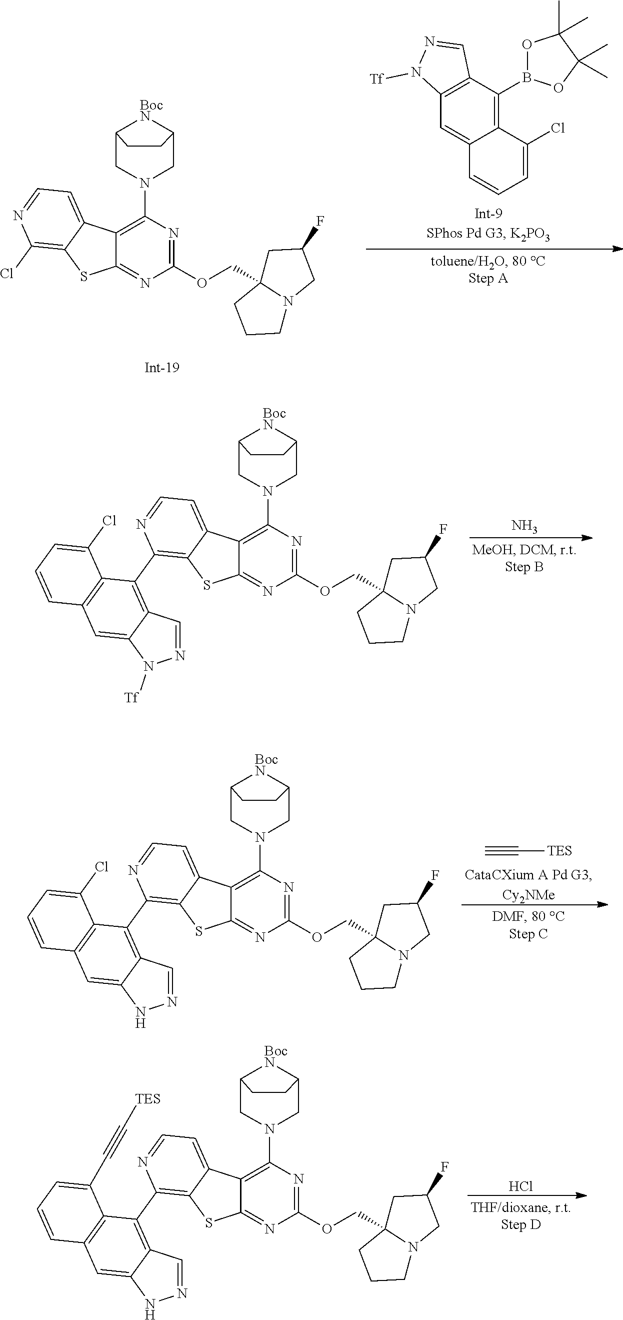

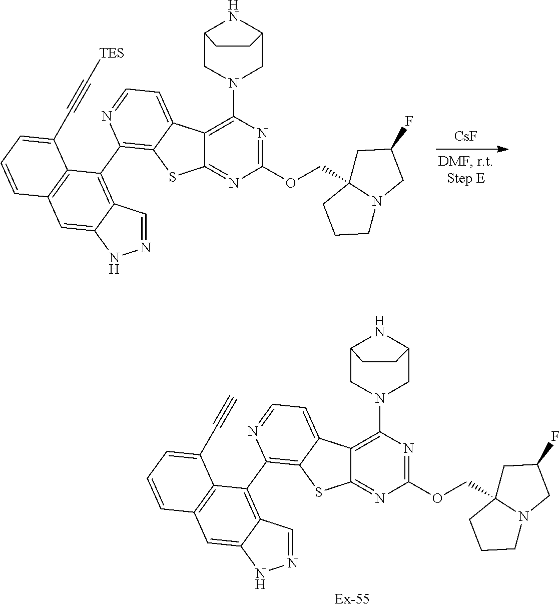

### Step A: tert-butyl (1R,5S)-3-(8-(5-chloro-1-((trifluoromethyl)sulfonyl)-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate

In a glovebox, to a solution of tert-butyl (1R,5S)-3-(8-chloro-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate (Int-19) (120 mg, 0.204 mmol) and 5-chloro-4-(4,4,5,5-tetramethyl-1,3,2-dioxaborolan-2-yl)-1-((trifluoromethyl)sulfonyl)-1H-benzo[f]indazole (Int-9) (103 mg, 0.224 mmol) in toluene (5 mL) was added SPhos Pd G3 (79 mg, 0.102 mmol) and K₂CO₃ (0.306 mL, 0.611 mmol, 2M in H₂O) and the mixture was heated to 80° C. The reaction was stirred at 80° C. for 10 h. The reaction mixture was cooled to room temperature and diluted with EtOAc (20 mL). The mixture was washed with H₂O (2 mL) and brine (3 mL). The organic phase was dried over Na₂SO₄, filtered, and concentrated in vacuo. The residue was purified by flash silica gel chromatography (petroleum ether/THF=1/1) to give tert-butyl (1R,5S)-3-(8-(5-chloro-1-((trifluoromethyl)sulfonyl)-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate. MS (ESI) [M+H]⁺: m/z 887.

### Step B: tert-butyl (1R,5S)-3-(8-(5-chloro-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate

A solution of tert-butyl (1R,5S)-3-(8-(5-chloro-1-((trifluoromethyl)sulfonyl)-1H-benzo[/]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate (110 mg, 0.124 mmol) in ammonia (2 mL, 14.0 mmol, 7 M in MeOH) and DCM (0.5 mL) was stirred at 25° C. for 2 h. The mixture was concentrated in vacuo and the residue was purified by preparative TLC (2/3=petroleum ether/THF (w/1% NH₃ (7M in MeOH))) to give tert-butyl (1R,5S)-3-(8-(5-chloro-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate. MS (ESI) [M+H]⁺: m/z 755.

### Step C: tert-butyl (1R,5S)-3-(2-(((2R,7aS)-2-fluorotetrahydro-1H-pyrrolizin-7a(5H)-yl)methoxy)-8-(5-((triethylsilyl)ethynyl)-1H-benzo[f]indazol-4-yl)pyrido[4′,3′:4,5]thieno[2,3-d]pyrimidin-4-yl)-3,8-diazabicyclo[3.2.1]octane-8-carboxylate

In a glovebox, to a solution of tert-butyl (1R,5S)-3-(8-(5-chloro-1H-benzo[/]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate (70 mg, 0.093 mmol) in DMF (2 mL) was added N,N-dicyclohexylmethylamine (72.4 mg, 0.371 mmol), triethyl(ethynyl)silane (130 mg, 0.927 mmol) and cataCXium A Pd G3 (33.8 mg, 0.046 mmol). The reaction mixture was heated to 80° C. and stirred for 16 h. After 16 h, the reaction was cooled to room temperature and concentrated in vacuo. The residue was diluted with EtOAc (5 mL), washed with brine (1 mL), and the organic layer was concentrated in vacuo. The residue was purified by preparative TLC (petroleum ether/THF (SiO₂, contained 1% NH₃, 7M in MeOH)=2/3) to give tert-butyl (1R,5S)-3-(2-(((2R,7aS)-2-fluorotetrahydro-1H-pyrrolizin-7a(5H)-yl)methoxy)-8-(5-((triethylsilyl)ethynyl)-1H-benzo[f]indazol-4-yl)pyrido[4′,3′:4,5]thieno[2,3-d]pyrimidin-4-yl)-3,8-diazabicyclo[3.2.1]octane-8-carboxylate. MS (ESI) [M+H]⁺: m/z 859.

### Step D: 4-((1R,5S)-3,8-diazabicyclo[3.2.1]octan-3-yl)-2-(((2R,7aS)-2-fluorotetrahydro-1H-pyrrolizin-7a(5H)-yl)methoxy)-8-(5-((triethylsilyl)ethynyl)-1H-benzo[f]indazol-4-yl)pyrido[4′,3′:4,5]thieno[2,3-d]pyrimidine

To a solution of tert-butyl (1R,5S)-3-(2-(((2R,7aS)-2-fluorotetrahydro-1H-pyrrolizin-7a(5H)-yl)methoxy)-8-(5-((triethylsilyl)ethynyl)-1H-benzo[f]indazol-4-yl)pyrido[4′,3′:4,5]thieno[2,3-d]pyrimidin-4-yl)-3,8-diazabicyclo[3.2.1]octane-8-carboxylate (45 mg, 0.052 mmol) in THF (0.5 mL) was added HCl (1 mL, 4.00 mmol, 4 M in dioxane) at 25° C. The mixture was stirred at 25° C. for 20 min. The reaction mixture was concentrated in vacuo to afford 4-((1R,5S)-3,8-diazabicyclo[3.2.1]octan-3-yl)-2-(((2R,7aS)-2-fluorotetrahydro-1H-pyrrolizin-7a(5H)-yl)methoxy)-8-(5-((triethylsilyl)ethynyl)-1H-benzo[f]indazol-4-yl)pyrido[4′,3′:4,5]thieno[2,3-d]pyrimidine. MS (ESI) [M+H]⁺: m/z 759.

### Step E: 4-((1R,5S)-3,8-diazabicyclo[3.2.1]octan-3-yl)-8-(5-ethynyl-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidine (Ex-55)

To a solution of 4-((1R,5S)-3,8-diazabicyclo[3.2.1]octan-3-yl)-2-(((2R,7aS)-2-fluorotetrahydro-1H-pyrrolizin-7a(5H)-yl)methoxy)-8-(5-((triethylsilyl)ethynyl)-1H-benzo[/]indazol-4-yl)pyrido[4′,3′:4,5]thieno[2,3-d]pyrimidine (39.8 mg, 0.052 mmol) in DMF (1 mL) was added CsF (39.8 mg, 0.262 mmol) at 25° C. The mixture was stirred at 25° C. for 16 h. The reaction mixture was filtered and the filtrate was concentrated in vacuo. The residue was purified by preparative reverse-phase HPLC (MeCN/H₂O w/NH₄OH+NH₄HCO₃ modifier) to give 4-((1R,5S)-3,8-diazabicyclo[3.2.1]octan-3-yl)-8-(5-ethynyl-1H-benzo[/]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidine (Ex-55). MS (ESI) [M+H]⁺: m/z 645. ¹H NMR (400 MHz, MeOD) δ 8.69 (d, J=5.72 Hz, 1H), 8.30 (s, 1H), 8.16 (d, J=8.46 Hz, 1H), 7.86 (d, J=5.72 Hz, 1H), 7.80 (s, 1H), 7.61 (d, J=6.91 Hz, 1H), 7.45 (dd, J=7.03, 8.46 Hz, 1H), 5.17-5.35 (m, 1H), 4.42 (br s, 1H), 4.12-4.26 (m, 3H), 3.71-3.82 (m, 1H), 3.56-3.67 (m, 3H), 3.17-3.24 (m, 2H), 3.14 (s, 1H), 2.94-3.02 (m, 2H), 2.12-2.30 (m, 2H), 2.07 (br d, J=8.34 Hz, 1H), 1.92-2.02 (m, 2H), 1.82-1.89 (m, 2H), 1.73-1.80 (m, 2H), 1.63-1.71 (m, 1H).

The examples in the table below were synthesized using a similar procedure as described in the synthesis of Ex-55 by making the appropriate substitutions for starting material, intermediates, and/or reagents. Appropriate substitutions are available commercially, synthesized as described in the literature, synthesized using methods available to those skilled in the art, or synthesized as described herein.

| | | | | [M + |
|---|---|---|---|---|
| | | | | H]⁺ |
| Ex. | Starting material | Structure | Compound Name | Found |
| Ex- 56 | 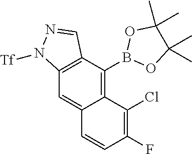 Int-10 | 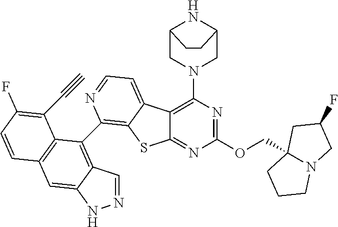 [SMILES: C#Cc1c(F)ccc2cc3[nH]ncc3c(-c3nccc4c3sc3nc(OC[C@@]56CCCN5C[C@H](F)C6)nc(N5CC6CCC(C5)N6)c34)c12] | 4-((1R,5S)-3,8- diazabicyclo[3.2.1]octan-3- yl)-8-(5-ethyny1-6-fluoro- 1H-benzo[f]indazol-4- yl)-2-(((2R,7aS)-2- fluorotetrahydro-1H- pyrrolizin-7a(5H)-yl) methoxy)pyrido[4′,3′:4,5] thieno[2,3-d\|pyrimidine | 663 |
| Ex- 57 | Byproduct of Ex-56 | 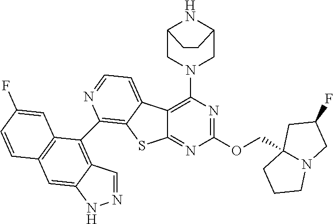 [SMILES: Fc1ccc2cc3[nH]ncc3c(-c3nccc4c3sc3nc(OC[C@@]56CCCN5C[C@H](F)C6)nc(N5CC6CCC(C5)N6)c34)c2c1] | 4-((1R,5S)-3,8- diazabicyclo[3.2.1]octan- 3-yl)-8-(6-fluoro-1H- benzo[f]indazol-4-yl)-2- (((2R,7aS)-2- fluorotetrahydro-1H- pyrrolizin-7a(5H)- yl)methoxy)pyri- do[4′,3′:4,5] thieno[2,3-d]pyrimidine | 639 |

### Example 58: 4-((1R,5S)-3,8-diazabicyclo[3.2.1]octan-3-yl)-8-(5-chloro-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidine (Ex-58)

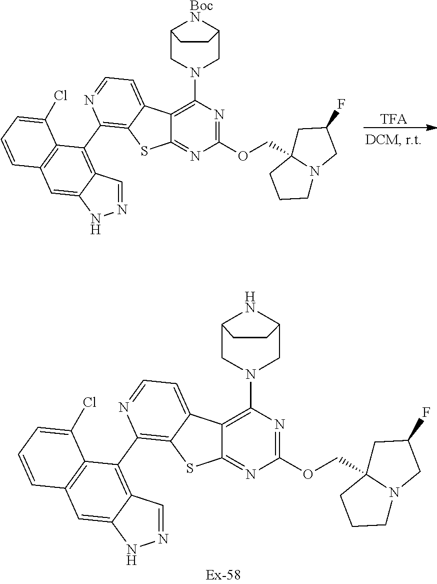 [SMILES: CC(C)(C)OC(=O)N1C2CCC1CN(c1nc(OC[C@@]34CCCN3C[C@H](F)C4)nc3sc4c(-c5c6cn[nH]c6cc6cccc(Cl)c56)nccc4c13)C2 ; F[C@H]1CN2CCC[C@@]2(COc2nc(N3CC4CCC(C3)N4)c3c(n2)sc2c(-c4c5cn[nH]c5cc5cccc(Cl)c45)nccc23)C1 ; "TFA"]

To a solution of tert-butyl (1R,5S)-3-(8-(5-chloro-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate (Step B of Example 55) (10 mg, 0.013 mmol) in DCM (0.25 mL) was added TFA (0.75 mL) and the mixture was stirred at 25° C. for 15 min. The mixture was concentrated in vacuo and the residue was diluted with MeCN (1 mL). The mixture was basified with NH₄OH, filtered, and the filtrate was purified by preparative reverse-phase HPLC (MeCN/H₂O w/NH₄OH+NH₄HCO₃ modifier) to give 4-((1R,5S)-3,8-diazabicyclo[3.2.1]octan-3-yl)-8-(5-chloro-1H-benzo[/]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidine (Ex-58). ¹H NMR (400 MHz, MeOD) δ 8.63 (d, J=5.7 Hz, 1H), 8.25 (s, 1H), 8.02 (dd, J=1.4, 8.1 Hz, 1H), 7.75 (d, J=5.5 Hz, 1H), 7.61 (s, 1H), 7.39-7.29 (m, 2H), 5.28-5.09 (m, 1H), 4.43-4.33 (m, 1H), 4.20-4.06 (m, 3H), 3.71 (br d, J=10.5 Hz, 1H), 3.65-3.51 (m, 3H), 3.18-3.05 (m, 3H), 2.92 (dt, J=5.7, 9.4 Hz, 1H), 2.17-2.07 (m, 2H), 2.04-1.94 (m, 1H), 1.93-1.82 (m, 3H), 1.81-1.62 (m, 4H). MS (ESI) [M+H]⁺: m/z 655.

The example in the table below was synthesized using a similar procedure as described in the synthesis of Ex-58 by making the appropriate substitutions for starting material, intermediates, and/or reagents. Appropriate substitutions are available commercially, synthesized as described in the literature, synthesized using methods available to those skilled in the art, or synthesized as described herein.

| | | | | [M + H]⁺ |
|---|---|---|---|---|
| Ex. | Starting material | Structure | Compound Name | Found |
| Ex- 59 | 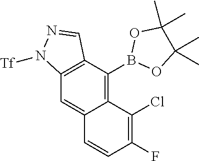 Int-10 | 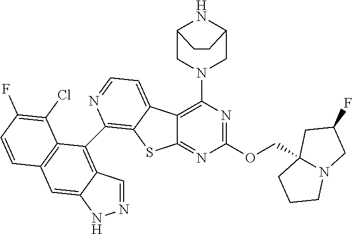 [SMILES: Fc1ccc2cc3[nH]ncc3c(-c3nccc4c3sc3nc(OC[C@@]56CCCN5C[C@H](F)C6)nc(N5CC6CCC(C5)N6)c34)c2c1Cl] | 4-((1R,5S)-3,8- diazabicyclo[3.2.1]octan-3- yl)-8-(5-chloro-6-fluoro-1H- benzo[f]indazol-4-yl)-2- (((2R,7aS)-2- fluorotetrahydro-1H- pyrrolizin-7a(5H)-yl) methoxy)pyrido[4′,3′:4,5]thi- eno[2,3-d]pyrimidine | 673 |

### Example 60: 4-(4-(3,8-diazabicyclo[3.2.1]octan-3-yl)-2-(((R)-2,2-difluoro-1-(hydroxymethyl)cyclopropyl)methoxy)pyrido[4′,3′:4,5]thieno[2,3-d]pyrimidin-8-yl)-2-amino-7-fluorobenzo[b]thiophene-3-carbonitrile (Ex-60)

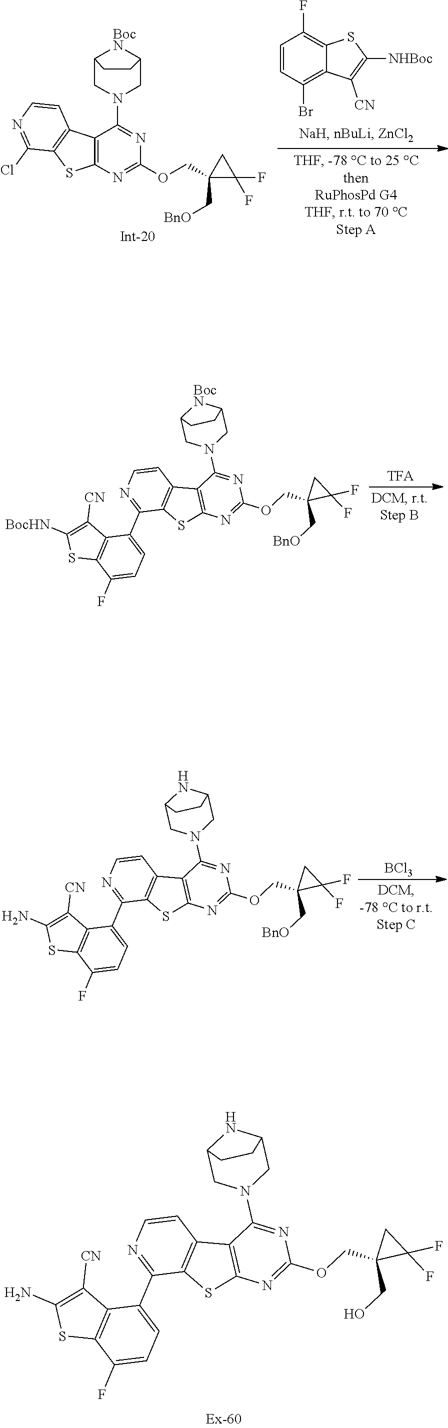 [SMILES: CC(C)(C)OC(=O)N1C2CCC1CN(c1nc(OC[C@]3(COCc4ccccc4)CC3(F)F)nc3sc4c(Cl)nccc4c13)C2 ; CC(C)(C)OC(=O)Nc1sc2c(F)ccc(Br)c2c1C#N ; CC(C)(C)OC(=O)Nc1sc2c(F)ccc(-c3nccc4c3sc3nc(OC[C@]5(COCc6ccccc6)CC5(F)F)nc(N5CC6CCC(C5)N6C(=O)OC(C)(C)C)c34)c2c1C#N ; N#Cc1c(N)sc2c(F)ccc(-c3nccc4c3sc3nc(OC[C@]5(COCc6ccccc6)CC5(F)F)nc(N5CC6CCC(C5)N6)c34)c12 ; "BCl3" ; N#Cc1c(N)sc2c(F)ccc(-c3nccc4c3sc3nc(OC[C@]5(CO)CC5(F)F)nc(N5CC6CCC(C5)N6)c34)c12]

### Step A: Tert-butyl 3-(2-(((R)-1-((benzyloxy)methyl)-2,2-difluorocyclopropyl)methoxy)-8-(2-((tert-butoxycarbonyl)amino)-3-cyano-7-fluorobenzo[b]thiophen-4-yl)pyrido[4′,3′:4,5]thieno[2,3-d]pyrimidin-4-yl)-3,8-diazabicyclo[3.2.1]octane-8-carboxylate

A vial was charged with tert-butyl (4-bromo-3-cyano-7-fluorobenzo[b]thiophen-2-yl)carbamate (37 mg, 0.1 mmol) and brought into a glovebox. The compound was dissolved in THF (1 mL) and sodium hydride (2.80 mg, 0.105 mmol) was added. The reaction was stirred at room temperature for 10 minutes. The reaction was then cooled to −78° C. whereupon n-BuLi (42.0 μl, 0.105 mmol, 2.5 M in hexanes) was added. The reaction stirred at −78° C. for 30 minutes. After 30 minutes, ZnCl₂ (105 μL, 0.105 mmol, 1M in THF) was added via syringe. The reaction was allowed to warm to room temperature over 30 minutes. In a separate vial, tert-butyl (1R,5S)-3-(2-(((R)-1-((benzyloxy)methyl)-2,2-difluorocyclopropyl)methoxy)-8-chloropyrido[4′,3′:4,5]thieno[2,3-d]pyrimidin-4-yl)-3,8-diazabicyclo[3.2.1]octane-8-carboxylate (Int-20) (66 mg, 0.10 mmol) and RuPhos Pd G4 (8.5 mg, 0.01 mmol) were dissolved in THF (1 mL). The substrate mixture was added to the freshly prepared zincate solution via syringe and the reaction was heated at 70° C. for 17 hours. The reaction was cooled to room temperature and quenched with sat. aq. NH₄Cl, extracted with DCM (3×), and concentrated under reduced pressure. The reaction was purified by silica gel column chromatography (0 to 100% EtOAc in hexanes) to afford tert-butyl (1R,5S)-3-(2-(((R)-1-((benzyloxy)methyl)-2,2-difluorocyclopropyl)methoxy)-8-(2-((tert-butoxycarbonyl)amino)-3-cyano-7-fluorobenzo[b]thiophen-4-yl)pyrido[4′,3′:4,5]thieno[2,3-d]pyrimidin-4-yl)-3,8-diazabicyclo[3.2.1]octane-8-carboxylate. MS (ESI) [M+H]⁺: 914.

### Step B: 2-amino-4-(2-(((R)-1-((benzyloxy)methyl)-2,2-difluorocyclopropyl)methoxy)-4-(3,8-diazabicyclo[3.2.1]octan-3-yl)pyrido[4′,3′:4,5]thieno[2,3-d]pyrimidin-8-yl)-7-fluorobenzo[b]thiophene-3-carbonitrile

A vial with stir bar was charged with tert-butyl (1R,5S)-3-(2-(((R)-1-((benzyloxy)methyl)-2,2-difluorocyclopropyl)methoxy)-8-(2-((tert-butoxycarbonyl)amino)-3-cyano-7-fluorobenzo[b]thiophen-4-yl)pyrido[4′,3′:4,5]thieno[2,3-d]pyrimidin-4-yl)-3,8-diazabicyclo[3.2.1]octane-8-carboxylate (32 mg, 0.04 mmol), DCM (519 μL), and TFA (173 μL). The reaction was stirred at room temperature for 30 min. The reaction was quenched with sat. aq. NaHCO₃, extracted with EtOAc (3×), dried over Na₂SO₄, filtered, and concentrated in vacuo. The resulting crude mixture including 2-amino-4-(2-(((R)-1-((benzyloxy)methyl)-2,2-difluorocyclopropyl)methoxy)-4-(3,8-diazabicyclo[3.2.1]octan-3-yl)pyrido[4′,3′:4,5]thieno[2,3-d]pyrimidin-8-yl)-7-fluorobenzo[b]thiophene-3-carbonitrile was used directly in the next step without further purification. MS (ESI) [M+H]⁺: 714.

### Step C: 4-(4-(3,8-diazabicyclo[3.2.1]octan-3-yl)-2-(((R)-2,2-difluoro-1-(hydroxymethyl)cyclopropyl)methoxy)pyrido[4′,3′:4,5]thieno[2,3-d]pyrimidin-8-yl)-2-amino-7-fluorobenzo[b]thiophene-3-carbonitrile (Ex-60)

An oven-dried vial with stir bar was charged with 2-amino-4-(2-(((R)-1-((benzyloxy)methyl)-2,2-difluorocyclopropyl)methoxy)-4-(3,8-diazabicyclo[3.2.1]octan-3-yl)pyrido[4′,3′:4,5]thieno[2,3-d]pyrimidin-8-yl)-7-fluorobenzo[b]thiophene-3-carbonitrile (32 mg, 0.04 mmol) and DCM (443 μL). The reaction mixture was cooled to −78° C. BCl₃ (133 μL, 0.133 mmol, 1 M in DCM) was added and reaction stirred at −78° C. for 1 hr then warmed to room temperature for 1 hr. The reaction was quenched with MeOH (0.5 mL) and concentrated in vacuo. The residue was taken up in DMSO (1.0 mL), filtered, and purified by reverse-phase HPLC (MeCN/water with NH₄OH modifier) to afford 4-(4-(3,8-diazabicyclo[3.2.1]octan-3-yl)-2-(((R)-2,2-difluoro-1-(hydroxymethyl)cyclopropyl)methoxy)pyrido[4′,3′:4,5]thieno[2,3-d]pyrimidin-8-yl)-2-amino-7-fluorobenzo[b]thiophene-3-carbonitrile (Ex-60). MS (ESI) [M+H]⁺: 624. ¹H NMR (600 MHz, d6-DMSO) δ 8.65 (d, J=5.5 Hz, 1H), 8.02 (s, 2H), 7.67 (d, J=5.6 Hz, 1H), 7.46 (dd, J=8.3, 5.3 Hz, 1H), 7.19-7.12 (m, 1H), 5.08 (t, J=5.6 Hz, 1H), 4.47 (m, 2H), 4.05 (d, J=11.6 Hz, 2H), 3.61-3.54 (m, 4H), 3.46 (s, 2H), 1.72-1.64 (m, 1H), 1.56 (m, 5H).

The examples in the table below were synthesized using a similar procedure as described in the synthesis of Ex-60 by making the appropriate substitutions for starting material, intermediates, and/or reagents. Appropriate substitutions are available commercially, synthesized as described in the literature, synthesized using methods available to those skilled in the art, or synthesized as described herein.

| Ex. | Starting material | Structure | Compound Name | [M + H]⁺ Found |
|---|---|---|---|---|
| Ex-61 | 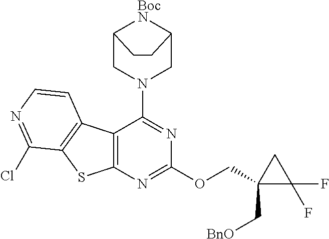 [SMILES: CC(C)(C)OC(=O)N1C2CCC1CN(c1nc(OC[C@]3(COCc4ccccc4)CC3(F)F)nc3sc4c(Cl)nccc4c13)C2] Int-20 | 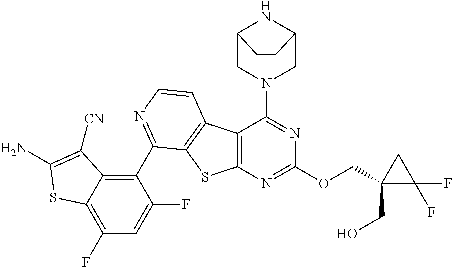 [SMILES: N#Cc1c(N)sc2c(F)cc(F)c(-c3nccc4c3sc3nc(OC[C@]5(CO)CC5(F)F)nc(N5CC6CCC(C5)N6)c34)c12] | 4-(4-((1R,5S)-3,8- diazabicyclo[3.2.1]octan- 3-yl)-2-(((R)-2,2- difluoro-1- (hydroxymethyl)cyclopro- pyl)methoxy)pyrido[4′,3′: 4,5]thieno[2,3- d]pyrimidin-8-yl)-2- amino-5,7- difluorobenzo[b]thiophene- 3-carbonitrile | 642 |
| Ex-62 | 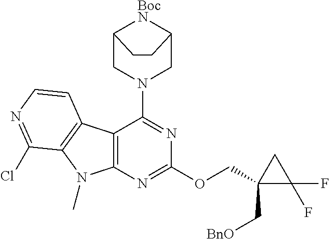 [SMILES: Cn1c2nc(OC[C@]3(COCc4ccccc4)CC3(F)F)nc(N3CC4CCC(C3)N4C(=O)OC(C)(C)C)c2c2ccnc(Cl)c21] Int-21 | 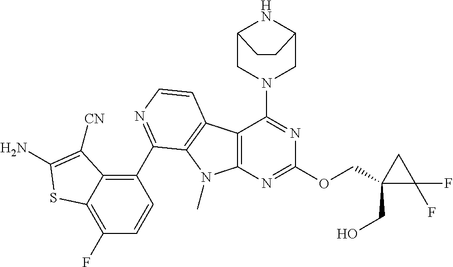 [SMILES: Cn1c2nc(OC[C@]3(CO)CC3(F)F)nc(N3CC4CCC(C3)N4)c2c2ccnc(-c3ccc(F)c4sc(N)c(C#N)c34)c21] | 4-(4-((1R,5S)-3,8- diazabicyclo[3.2.1]octan- 3-yl)-2-(((R)-2,2- difluoro-1- (hydroxymethyl)cyclopro- pyl)methoxy)-9-methyl- 9H- pyrido[4′,3′:4,5]pyrrolo[2,3- d]pyrimidin-8-yl)-2- amino-7- fluorobenzo[b]thiophene- 3-carbonitrile | 621 |
| Ex-63 | 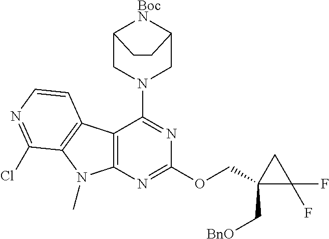 [SMILES: Cn1c2nc(OC[C@]3(COCc4ccccc4)CC3(F)F)nc(N3CC4CCC(C3)N4C(=O)OC(C)(C)C)c2c2ccnc(Cl)c21] Int-21 | 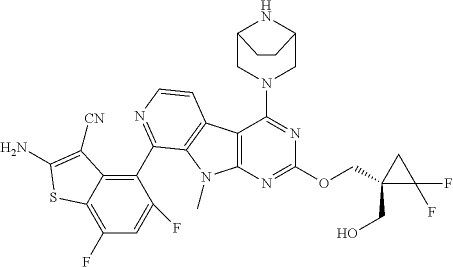 [SMILES: Cn1c2nc(OC[C@]3(CO)CC3(F)F)nc(N3CC4CCC(C3)N4)c2c2ccnc(-c3c(F)cc(F)c4sc(N)c(C#N)c34)c21] | 4-(4-((1R,5S)-3,8- diazabicyclo[3.2.1]octan- 3-yl)-2-(((R)-2,2- difluoro-1- (hydroxymethyl)cyclopro- pyl)methoxy)-9-methyl- 9H- pyrido[4′,3′:4,5]pyrrolo[2,3- d]pyrimidin-8-yl)-2- amino-5,7- difluorobenzo[b]thiophene- 3-carbonitrile | 639 |

### Example 64: (1-(((4-((1R,5S)-3,8-diazabicyclo[3.2.1]octan-3-yl)-8-(5-(difluoromethyl)-6-methyl-1H-indazol-4-yl)pyrido[4′,3′:4,5]thieno[2,3-d]pyrimidin-2-yl)oxy)methyl)cyclobutyl)methanol (Ex-64)

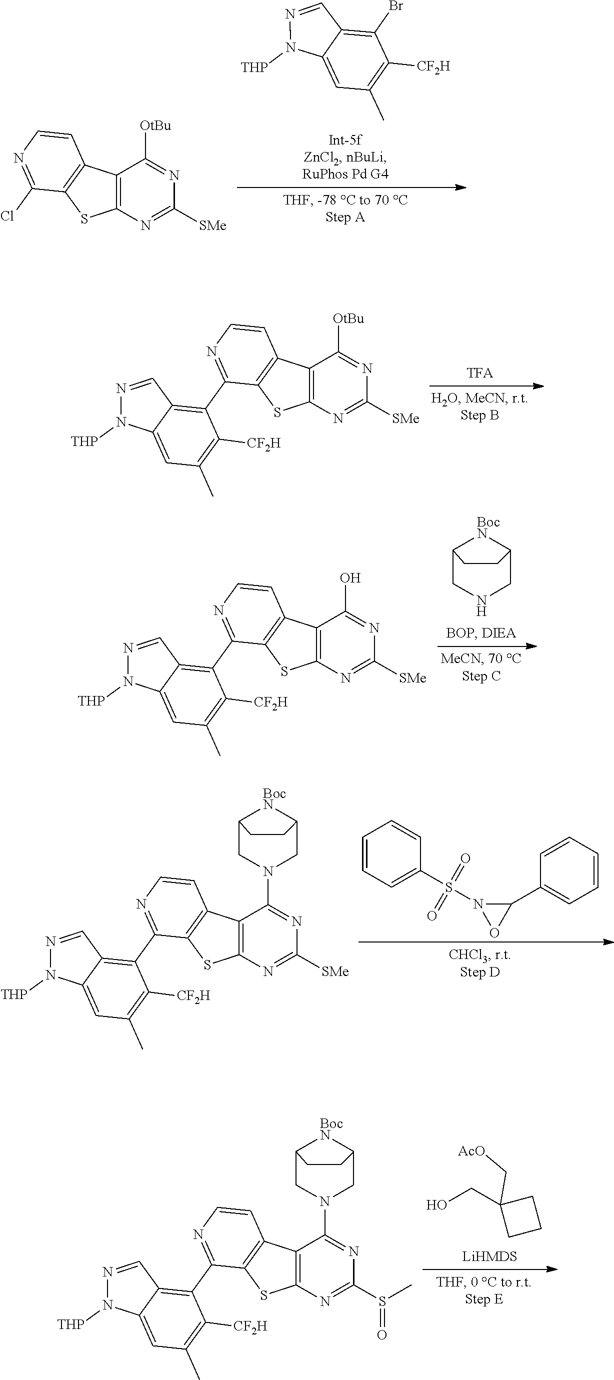

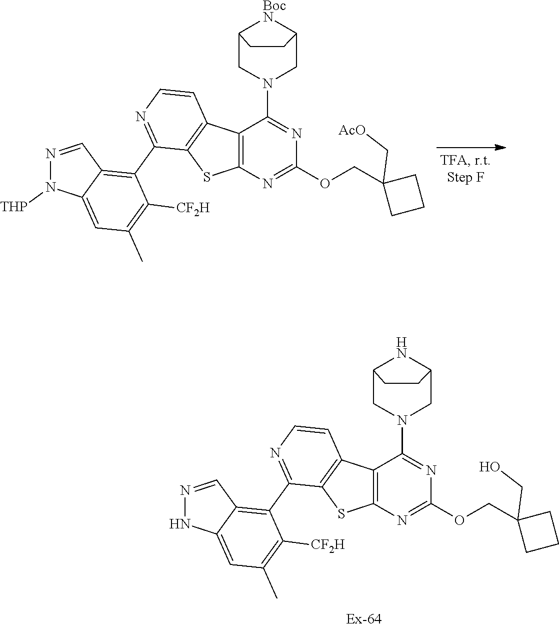

### Step A: 4-(tert-butoxy)-8-(5-(difluoromethyl)-6-methyl-1-(tetrahydro-2H-pyran-2-yl)-1H-indazol-4-yl)-2-(methylthio)pyrido[4′,3′:4,5]thieno[2,3-d]pyrimidine

4-bromo-5-(difluoromethyl)-6-methyl-1-(tetrahydro-2H-pyran-2-yl)-1H-indazole (Int-5F) (2.00 g, 5.79 mmol) was dissolved in 20 mL THF in a 3-neck round bottom flask. The contents of the flask were chilled to −78° C. and nBuLi (2.43 mL, 6.08 mmol, 2.5 M in hexanes) was added. The reaction mixture was stirred at −78° C. for 30 min, at which point ZnCl₂ (6.08 mL, 6.08 mmol, 1 M in THF) solution was added. The reaction mixture was allowed to warm to room temperature over 30 minutes. In a separate vial, 4-(tert-butoxy)-8-chloro-2-(methylthio)pyrido[4′,3′:4,5]thieno[2,3-d]pyrimidine (Step A of Example 20) (1.969 g, 5.79 mmol) and RuPhos Pd G4 (0.493 g, 0.579 mmol) were dissolved in 20 mL THF. The palladium solution was transferred to the reaction flask via syringe and the resulting mixture was heated to 70° C. for 48 hours. After 48 hours, the reaction mixture was cooled to room temperature, diluted with EtOAc, and washed with water. The aqueous layer was separated and extracted with EtOAc. The combined organic layers were washed with brine, dried with Na₂SO₄, filtered, and concentrated. The crude reaction mixture was purified via silica gel chromatography (0 to 40% EtOAc in hexanes) to obtain 4-(tert-butoxy)-8-(5-(difluoromethyl)-6-methyl-1-(tetrahydro-2H-pyran-2-yl)-1H-indazol-4-yl)-2-(methylthio)pyrido[4′,3′:4,5]thieno[2,3-d]pyrimidine. MS (ESI) [M+H]⁺: 570.

### Step B: 8-(5-(difluoromethyl)-6-methyl-1-(tetrahydro-2H-pyran-2-yl)-1H-indazol-4-yl)-2-(methylthio)pyrido[4′,3′:4,5]thieno[2,3-d]pyrimidin-4-ol

4-(tert-butoxy)-8-(5-(difluoromethyl)-6-methyl-1-(tetrahydro-2H-pyran-2-yl)-1H-indazol-4-yl)-2-(methylthio)pyrido[4′,3′:4,5]thieno[2,3-d]pyrimidine (1.00 g, 1.76 mmol) was taken up in a mixture of MeCN (13.8 mL), H₂O (3.44 mL), and TFA (0.688 mL). The resulting mixture was allowed to stir for 4 h at room temperature. The reaction was quenched with sat. aq. NaHCO₃, extracted with DCM (3×), and concentrated under reduced pressure. The crude mixture including 8-(5-(difluoromethyl)-6-methyl-1-(tetrahydro-2H-pyran-2-yl)-1H-indazol-4-yl)-2-(methylthio)pyrido[4′,3′:4,5]thieno[2,3-d]pyrimidin-4-ol was used directly in next step without further purification. MS (ESI) [M+H]⁺: 514.

### Step C: Tert-butyl (1R,5S)-3-(8-(5-(difluoromethyl)-6-methyl-1-(tetrahydro-2H-pyran-2-yl)-1H-indazol-4-yl)-2-(methylthio)pyrido[4′,3′:4,5]thieno[2,3-d]pyrimidin-4-yl)-3,8-diazabicyclo[3.2.1]octane-8-carboxylate

A solution of 8-(5-(difluoromethyl)-6-methyl-1-(tetrahydro-2H-pyran-2-yl)-1H-indazol-4-yl)-2-(methylthio)pyrido[4′,3′:4,5]thieno[2,3-d]pyrimidin-4-ol (747 mg, 1.454 mmol) in MeCN (14.5 mL) was added to a flask containing BOP (965 mg, 2.18 mmol). N,N-diisopropylethylamine (1.27 mL, 7.27 mmol) and tert-butyl (1R,5S)-3,8-diazabicyclo[3.2.1]octane-8-carboxylate (309 mg, 1.45 mmol) were added and the resulting mixture was heated to 70° C. for 5 h. The reaction was cooled to room temperature and quenched with sat. aq. NaHCO₃, extracted with DCM, and concentrated in vacuo. The crude reaction mixture was purified by silica gel column chromatography (0 to 100% EtOAc in hexanes) to afford tert-butyl (1R,5S)-3-(8-(5-(difluoromethyl)-6-methyl-1-(tetrahydro-2H-pyran-2-yl)-1H-indazol-4-yl)-2-(methylthio)pyrido[4′,3′:4,5]thieno[2,3-d]pyrimidin-4-yl)-3,8-diazabicyclo[3.2.1]octane-8-carboxylate. MS (ESI) [M+H]⁺: 708.

### Step D: Tert-butyl (1R,5S)-3-(8-(5-(difluoromethyl)-6-methyl-1-(tetrahydro-2H-pyran-2-yl)-1H-indazol-4-yl)-2-(methylsulfinyl)pyrido[4′,3′:4,5]thieno[2,3-d]pyrimidin-4-yl)-3,8-diazabicyclo[3.2.1]octane-8-carboxylate

Tert-butyl (1R,5S)-3-(8-(5-(difluoromethyl)-6-methyl-1-(tetrahydro-2H-pyran-2-yl)-1H-indazol-4-yl)-2-(methylthio)pyrido[4′,3′:4,5]thieno[2,3-d]pyrimidin-4-yl)-3,8-diazabicyclo[3.2.1]octane-8-carboxylate (620 mg, 0.876 mmol) was dissolved in CHCl₃ (8.76 mL) and 3-phenyl-2-(phenylsulfonyl)-1,2-oxaziridine (252 mg, 0.963 mmol) was added. The reaction was stirred at room temperature for 6 hours. The reaction was concentrated and purified by silica gel column chromatography (0 to 100% (3:1 EtOAc:EtOH) in hexanes) to afford tert-butyl (1R,5S)-3-(8-(5-(difluoromethyl)-6-methyl-1-(tetrahydro-2H-pyran-2-yl)-1H-indazol-4-yl)-2-(methylsulfinyl)pyrido[4′,3′:4,5]thieno[2,3-d]pyrimidin-4-yl)-3,8-diazabicyclo[3.2.1]octane-8-carboxylate. MS (ESI) [M+H]⁺: 724.

### Step E: Tert-butyl (1R,5S)-3-(2-((1-(acetoxymethyl)cyclobutyl)methoxy)-8-(5-(difluoromethyl)-6-methyl-1-(tetrahydro-2H-pyran-2-yl)-1H-indazol-4-yl)pyrido[4′3′:4,5]thieno[2,3-d]pyrimidin-4-yl)-3,8-diazabicyclo[3.2.1]octane-8-carboxylate

(1-(hydroxymethyl)cyclobutyl)methyl acetate (50 mg, 0.316 mmol) was dissolved in THF (316 μL) and the solution was chilled to 0° C. LiHMDS (316 μL, 0.316 mmol, 1M in THF) was added to this solution dropwise at 0° C. Once addition was complete, the mixture was warmed to room temperature and this solution was added to a solution of tert-butyl (1R,5S)-3-(8-(5-(difluoromethyl)-6-methyl-1-(tetrahydro-2H-pyran-2-yl)-1H-indazol-4-yl)-2-(methylsulfinyl)pyrido[4′,3′:4,5]thieno[2,3-d]pyrimidin-4-yl)-3,8-diazabicyclo[3.2.1]octane-8-carboxylate (35 mg, 0.048 mmol) in THF (484 μL). The resulting mixture was allowed to stir overnight at room temperature. The reaction was quenched with 1 mL of 20% v/v AcOH/PhMe. The reaction mixture was concentrated under reduced pressure to afford tert-butyl (1R,5S)-3-(2-((1-(acetoxymethyl)cyclobutyl)methoxy)-8-(5-(difluoromethyl)-6-methyl-1-(tetrahydro-2H-pyran-2-yl)-1H-indazol-4-yl)pyrido[4′,3′:4,5]thieno[2,3-d]pyrimidin-4-yl)-3,8-diazabicyclo[3.2.1]octane-8-carboxylate, which was used directly in the next step without further purification. MS (ESI) [M+H]⁺: 818.

### Step F: (1-(((4-((1R,5S)-3,8-diazabicyclo[3.2.1]octan-3-yl)-8-(5-(difluoromethyl)-6-methyl-1H-indazol-4-yl)pyrido[4′,3′:4,5]thieno[2,3-d]pyrimidin-2-yl)oxy)methyl)cyclobutyl)methanol (Ex-64)

TFA (3 mL) was added to a vial containing tert-butyl (1R,5S)-3-(2-((1-(acetoxymethyl)cyclobutyl)methoxy)-8-(5-(difluoromethyl)-6-methyl-1-(tetrahydro-2H-pyran-2-yl)-1H-indazol-4-yl)pyrido[4′,3′:4,5]thieno[2,3-d]pyrimidin-4-yl)-3,8-diazabicyclo[3.2.1]octane-8-carboxylate (39 mg, 0.048 mmol) and was stirred for 2 h at room temperature. The reaction mixture was concentrated under reduced pressure, reconstituted in 3 mL of MeCN/H₂O, and reconcentrated under reduced pressure. The resulting oil was dissolved in 3 mL of EtOH and 30% NH₄OH was added until reaching a pH>9. This mixture was heated to 40° C. and allowed to stir overnight. The reaction mixture was diluted with DMSO, filtered, and purified by preparative-scale reverse phase HPLC (MeCN/H₂O w/NH₄OH modifier) to afford (1-(((4-((1R,5S)-3,8-diazabicyclo[3.2.1]octan-3-yl)-8-(5-(difluoromethyl)-6-methyl-1H-indazol-4-yl)pyrido[4′,3′:4,5]thieno[2,3-d]pyrimidin-2-yl)oxy)methyl)cyclobutyl)methanol (Ex-64). MS (ESI) [M+H]⁺: 592. ¹H NMR (499 MHz, DMSO-d⁶) 6 8.74 (d, J=4.9 Hz, 1H), 7.74 (d, J=4.8 Hz, 1H), 7.67 (s, 1H), 7.61 (s, 1H), 6.69 (t, J=53.4 Hz, 1H), 4.73-4.67 (m, 1H), 4.32 (s, 2H), 4.14 (t, J=9.8 Hz, 2H), 3.65-3.57 (m, 2H), 3.53 (s, 2H), 3.48 (d, J=4.1 Hz, 2H), 2.70 (s, 3H), 1.90-1.79 (m, 6H), 1.62 (s, 4H).

### Example 65: 7-(2-(((2R,7aS)-2-fluorotetrahydro-1H-pyrrolizin-7a(5H)-yl)methoxy)-8-(6-methyl-5-(trifluoromethyl)-1H-indazol-4-yl)pyrido[4′,3′:4,5]thieno[2,3-d]pyrimidin-4-yl)-3-oxa-7,9-diazabicyclo[3.3.1]nonane (Ex-65)

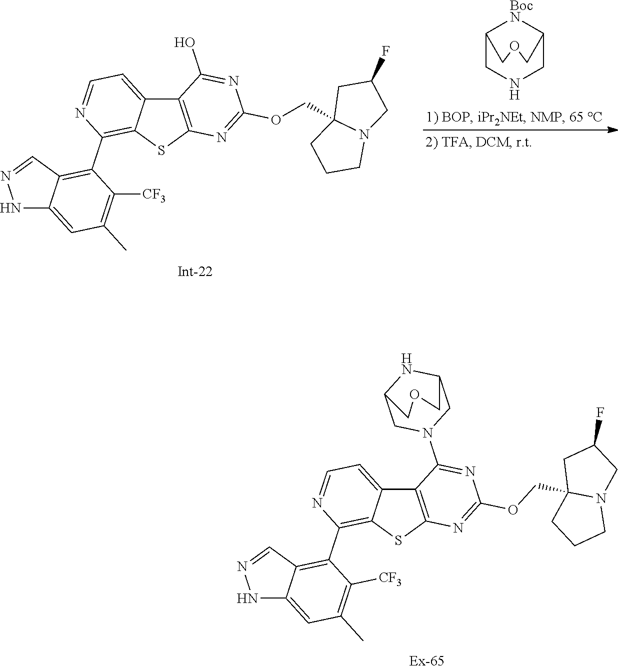 [SMILES: Cc1cc2[nH]ncc2c(-c2nccc3c2sc2nc(OC[C@@]45CCCN4C[C@H](F)C5)nc(O)c23)c1C(F)(F)F ; CC(C)(C)OC(=O)N1C2CNCC1COC2 ; Cc1cc2[nH]ncc2c(-c2nccc3c2sc2nc(OC[C@@]45CCCN4C[C@H](F)C5)nc(N4CC5COCC(C4)N5)c23)c1C(F)(F)F]

2-(((2R,7aS)-2-fluorotetrahydro-1H-pyrrolizin-7a(5H)-yl)methoxy)-8-(6-methyl-5-(trifluoromethyl)-1H-indazol-4-yl)pyrido[4′,3′:4,5]thieno[2,3-d]pyrimidin-4-ol (Int-22) (20 mg, 0.036 mmol), tert-butyl 3-oxa-7,9-diazabicyclo[3.3.1]nonane-9-carboxylate (24.5 mg, 0.107 mmol), and BOP (32 mg, 0.072 mmol) were added to a vial with stir bar. NMP (716 μL) and N,N-diisopropylethylamine (37.5 μL, 0.215 mmol) were added via syringe. The reaction was fitted with a septum and heated to 65° C. for 1.5 h. The reaction was cooled to room temperature and diluted with water (5 mL) and EtOAc (5 mL). The mixture was shaken and the layers were separated. The aqueous phase was washed with EtOAc (2×5 mL). The combined organic layers were dried over sodium sulfate, filtered, and concentrated. The crude reaction mixture was dissolved in DCM (1 mL) and TFA (0.3 mL) and allowed to stir for 1 h. The reaction was diluted with DCM (5 mL) and quenched with sat. aq. NaHCO₃ (5 mL). The layers were shaken and separated and the aqueous phase was washed with DCM (2×5 mL). The combined organic layers were washed with brine (10 mL), dried over sodium sulfate, filtered, and concentrated. The crude reaction mixture was purified by preparative reverse-phase HPLC (MeCN/H₂O, TFA modifier) to afford 7-(2-(((2R,7aS)-2-fluorotetrahydro-1H-pyrrolizin-7a(5H)-yl)methoxy)-8-(6-methyl-5-(trifluoromethyl)-1H-indazol-4-yl)pyrido[4′,3′:4,5]thieno[2,3-d]pyrimidin-4-yl)-3-oxa-7,9-diazabicyclo[3.3.1]nonane (Ex-64) as the 2TFA salt. [M+H]⁺: m/z 669. ¹H NMR (499 MHz, d₄-MeOD) δ 8.80 (d, J=5.3 Hz, 1H), 7.81 (d, J=5.2 Hz, 1H), 7.77 (s, 1H), 7.41 (s, 1H), 5.58 (d, J=51.5 Hz, 1H), 4.68 (s, 2H), 4.58 (s, 2H), 4.37 (m, 4H), 4.00-3.66 (m, 7H), 3.47 (m, 1H), 2.79-2.54 (m, 5H), 2.51-2.14 (m, 4H).

The examples in the table below were synthesized using a similar procedure as described in the synthesis of Ex-65 by making the appropriate substitutions for starting material, intermediates, and/or reagents. Appropriate substitutions are available commercially, synthesized as described in the literature, synthesized using methods available to those skilled in the art, or synthesized as described herein.

| Ex. | Starting material | Structure | Compound Name | [M + H]⁺ Found |
|---|---|---|---|---|
| Ex-66 | 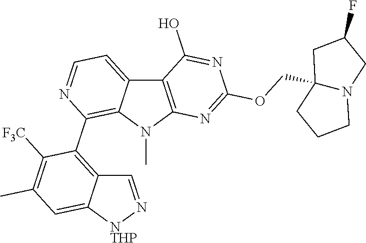 Int-18 | 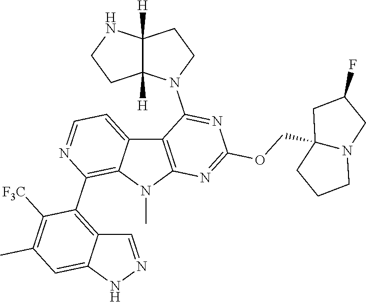 [SMILES: Cc1cc2[nH]ncc2c(-c2nccc3c4c(N5CC[C@H]6NCC[C@H]65)nc(OC[C@@]56CCCN5C[C@H](F)C6)nc4n(C)c23)c1C(F)(F)F] | 2-(((2R,7aS)-2- fluorotetrahydro-1H- pyrrolizin-7a(5H)- yl)methoxy)-4-((3aR,6aR)- hexahydropyrrolo[3,2- b]pyrrol-1(2H)-yl)-9- methyl-8-(6-methyl-5- (trifluoromethyl)-1H- indazol-4-yl)-9H- pyrido[4′,3′:4,5]pyrrolo[2,3- d]pyrimidine | 650 |
| Ex-67 | 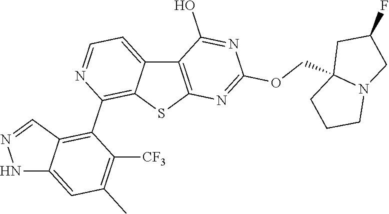 [SMILES: Cc1cc2[nH]ncc2c(-c2nccc3c2sc2nc(OC[C@@]45CCCN4C[C@H](F)C5)nc(O)c23)c1C(F)(F)F] Int-22 | 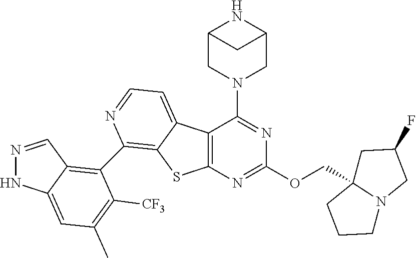 [SMILES: Cc1cc2[nH]ncc2c(-c2nccc3c2sc2nc(OC[C@@]45CCCN4C[C@H](F)C5)nc(N4CC5CC(C4)N5)c23)c1C(F)(F)F] | 4-(3,6- diazabicyclo[3.1.1]heptan- 3-yl)-2-(((2R,7aS)-2- fluorotetrahydro-1H- pyrrolizin-7a(5H)- yl)methoxy)-8-(6-methyl- 5-(trifluoromethyl)-1H- indazol-4- yl)pyrido[4′,3′:4,5]thieno[2,3- d]pyrimidine | 639 |
| Ex-68 | 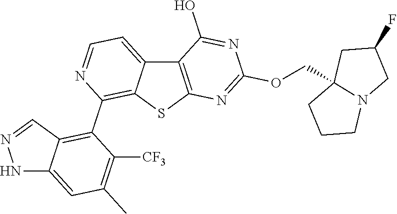 [SMILES: Cc1cc2[nH]ncc2c(-c2nccc3c2sc2nc(OC[C@@]45CCCN4C[C@H](F)C5)nc(O)c23)c1C(F)(F)F] Int-22 | 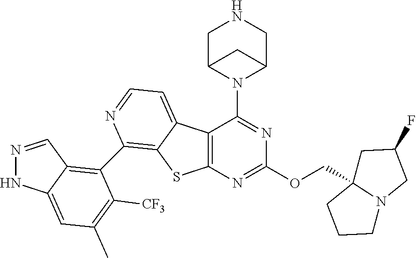 [SMILES: Cc1cc2[nH]ncc2c(-c2nccc3c2sc2nc(OC[C@@]45CCCN4C[C@H](F)C5)nc(N4C5CNCC4C5)c23)c1C(F)(F)F] | 4-(3,6- diazabicyclo[3.1.1]heptan- 6-yl)-2-(((2R,7aS)-2- fluorotetrahydro-1H- pyrrolizin-7a(5H)- yl)methoxy)-8-(6-methyl- 5-(trifluoromethyl)-1H- indazol-4- yl)pyrido[4′,3′:4,5]thieno[2,3- d]pyrimidine | 639 |
| Ex-69 | 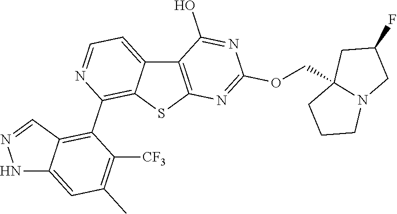 [SMILES: Cc1cc2[nH]ncc2c(-c2nccc3c2sc2nc(OC[C@@]45CCCN4C[C@H](F)C5)nc(O)c23)c1C(F)(F)F] Int-22 | 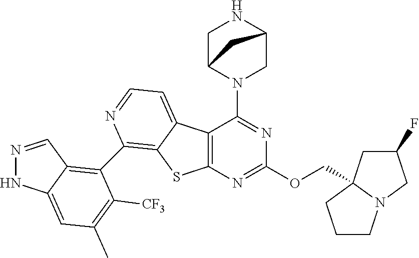 [SMILES: Cc1cc2[nH]ncc2c(-c2nccc3c2sc2nc(OC[C@@]45CCCN4C[C@H](F)C5)nc(N4C[C@@H]5C[C@H]4CN5)c23)c1C(F)(F)F] | 4-((1S,4S)-2,5- diazabicyclo[2.2.1]heptan- 2-yl)-2-(((2R,7aS)-2- fluorotetrahydro-1H- pyrrolizin-7a(5H)- yl)methoxy)-8-(6-methyl- 5-(trifluoromethyl)-1H- indazol-4- yl)pyrido[4′,3′:4,5]thieno[2,3- d]pyrimidine | 639 |
| Ex-70 | 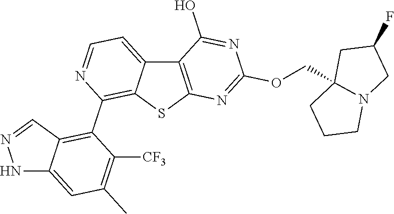 [SMILES: Cc1cc2[nH]ncc2c(-c2nccc3c2sc2nc(OC[C@@]45CCCN4C[C@H](F)C5)nc(O)c23)c1C(F)(F)F] Int-22 | 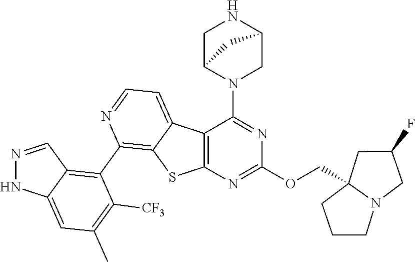 [SMILES: Cc1cc2[nH]ncc2c(-c2nccc3c2sc2nc(OC[C@@]45CCCN4C[C@H](F)C5)nc(N4C[C@H]5C[C@@H]4CN5)c23)c1C(F)(F)F] | 4-((1R,4R)-2,5- diazabicyclo[2.2.1]heptan- 2-yl)-2-(((2R,7aS)-2- fluorotetrahydro-1H- pyrrolizin-7a(5H)- yl)methoxy)-8-(6-methyl- 5-(trifluoromethyl)-1H- indazol-4- yl)pyrido[4′,3′:4,5]thieno[2,3- d]pyrimidine | 639 |
| Ex-71 | 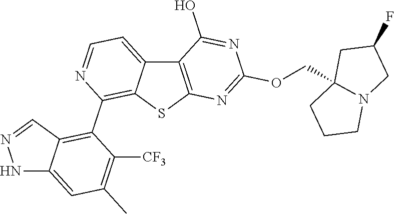 [SMILES: Cc1cc2[nH]ncc2c(-c2nccc3c2sc2nc(OC[C@@]45CCCN4C[C@H](F)C5)nc(O)c23)c1C(F)(F)F] Int-22 | 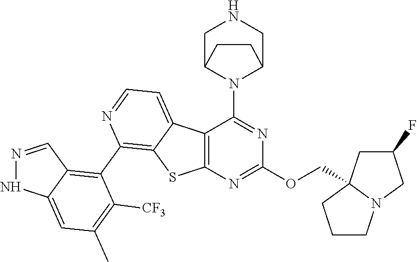 [SMILES: Cc1cc2[nH]ncc2c(-c2nccc3c2sc2nc(OC[C@@]45CCCN4C[C@H](F)C5)nc(N4C5CCC4CNC5)c23)c1C(F)(F)F] | 4-((1R,5S)-3,8- diazabicyclo[3.2.1]octan- 8-yl)-2-(((2R,7aS)-2- fluorotetrahydro-1H- pyrrolizin-7a(5H)- yl)methoxy)-8-(6-methyl- 5-(trifluoromethyl)-1H- indazol-4- yl)pyrido[4′,3′:4,5]thieno[2,3- d]pyrimidine | 653 |
| Ex-72 | 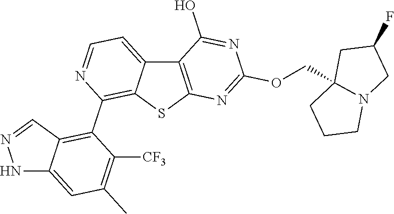 [SMILES: Cc1cc2[nH]ncc2c(-c2nccc3c2sc2nc(OC[C@@]45CCCN4C[C@H](F)C5)nc(O)c23)c1C(F)(F)F] Int-22 | 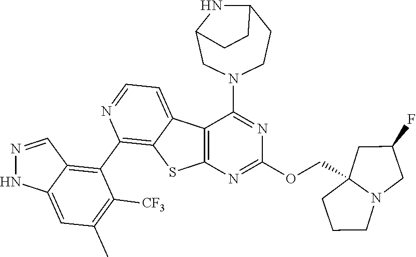 [SMILES: Cc1cc2[nH]ncc2c(-c2nccc3c2sc2nc(OC[C@@]45CCCN4C[C@H](F)C5)nc(N4CCC5CCC(C4)N5)c23)c1C(F)(F)F] | 4-(3,9- diazabicyclo[4.2.1]nonan- 3-yl)-2-(((2R,7aS)-2- fluorotetrahydro-1H- pyrrolizin-7a(5H)- yl)methoxy)-8-(6-methyl- 5-(trifluoromethyl)-1H- indazol-4- yl)pyrido[4′,3′:4,5]thieno[2,3- d]pyrimidine | 667 |
| Ex-73 | 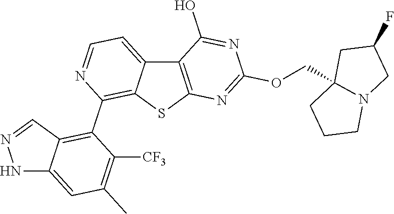 [SMILES: Cc1cc2[nH]ncc2c(-c2nccc3c2sc2nc(OC[C@@]45CCCN4C[C@H](F)C5)nc(O)c23)c1C(F)(F)F] Int-22 | 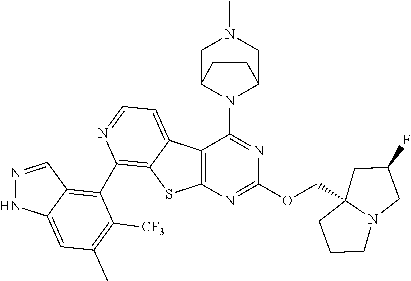 [SMILES: Cc1cc2[nH]ncc2c(-c2nccc3c2sc2nc(OC[C@@]45CCCN4C[C@H](F)C5)nc(N4C5CCC4CN(C)C5)c23)c1C(F)(F)F] | 2-(((2R,7aS)-2- fluorotetrahydro-1H- pyrrolizin-7a(5H)- yl)methoxy)-8-(6-methyl- 5-(trifluoromethyl)-1H- indazol-4-yl)-4-((1R,5S)-3- methyl-3,8- diazabicyclo[3.2.1]octan- 8- yl)pyrido[4′,3′:4,5]thieno[2,3- d]pyrimidine | 667 |
| Ex-74 | 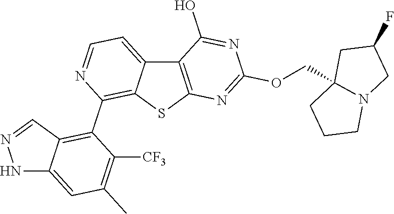 [SMILES: Cc1cc2[nH]ncc2c(-c2nccc3c2sc2nc(OC[C@@]45CCCN4C[C@H](F)C5)nc(O)c23)c1C(F)(F)F] Int-22 | 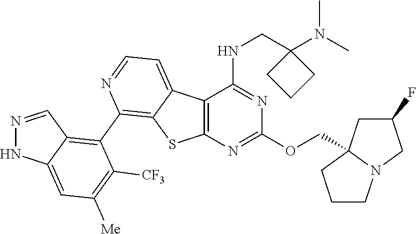 [SMILES: Cc1cc2[nH]ncc2c(-c2nccc3c2sc2nc(OC[C@@]45CCCN4C[C@H](F)C5)nc(NCC4(N(C)C)CCC4)c23)c1C(F)(F)F] | N-((1- (dimethylamino)cyclobutyl) methyl)-2-(((2R,7aS)-2- fluorotetrahydro-1H- pyrrolizin-7a(5H)- yl)methoxy)-8-(6-methyl- 5-(trifluoromethyl)-1H- indazol-4- yl)pyrido[4′,3′:4,5]thieno[2,3- d]pyrimidin-4-amine | 669 |
| Ex-75 | 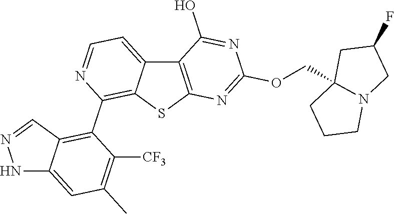 [SMILES: Cc1cc2[nH]ncc2c(-c2nccc3c2sc2nc(OC[C@@]45CCCN4C[C@H](F)C5)nc(O)c23)c1C(F)(F)F] Int-22 | 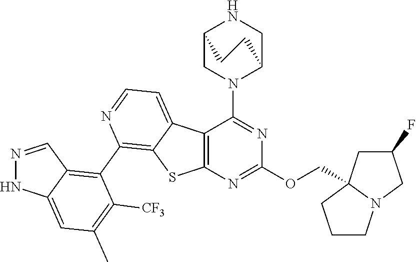 [SMILES: Cc1cc2[nH]ncc2c(-c2nccc3c2sc2nc(OC[C@@]45CCCN4C[C@H](F)C5)nc(N4C[C@@H]5CC[C@H]4CN5)c23)c1C(F)(F)F] | 4-((1S,4S)-2,5- diazabicyclo[2.2.2]octan- 2-yl)-2-(((2R,7aS)-2- fluorotetrahydro-1H- pyrrolizin-7a(5H)- yl)methoxy)-8-(6-methyl- 5-(trifluoromethyl)-1H- indazol-4- yl)pyrido[4′,3′:4,5]thieno[2,3- d]pyrimidine | 653 |
| Ex-76 | 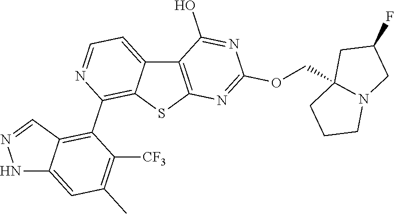 [SMILES: Cc1cc2[nH]ncc2c(-c2nccc3c2sc2nc(OC[C@@]45CCCN4C[C@H](F)C5)nc(O)c23)c1C(F)(F)F] Int-22 | 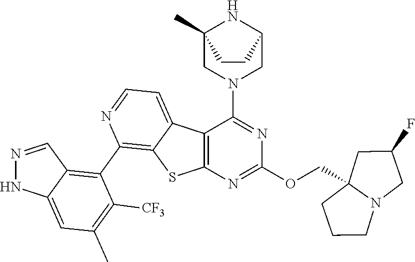 [SMILES: Cc1cc2[nH]ncc2c(-c2nccc3c2sc2nc(OC[C@@]45CCCN4C[C@H](F)C5)nc(N4C[C@H]5CC[C@@](C)(C4)N5)c23)c1C(F)(F)F] | 2-(((2R,7aS)-2- fluorotetrahydro-1H- pyrrolizin-7a(5H)- yl)methoxy)-8-(6-methyl- 5-(trifluoromethyl)-1H- indazol-4-yl)-4-((1S,5R)-1- methyl-3,8- diazabicyclo[3.2.1]octan- 3- yl)pyrido[4′,3′:4,5]thieno[2,3- d]pyrimidine | 667 |

### Example 77: 4-(3,8-diazabicyclo[3.2.1]octan-3-yl)-2-(((2R,7aS)-2-fluorotetrahydro-1H-pyrrolizin-7a(5H)-yl)methoxy)-8-(6-methyl-5-(trifluoromethyl)-TH-indazol-4-yl)pyrimildo[5′,4′:4,5]thieno[2,3-d]pyridazine (Ex-77)

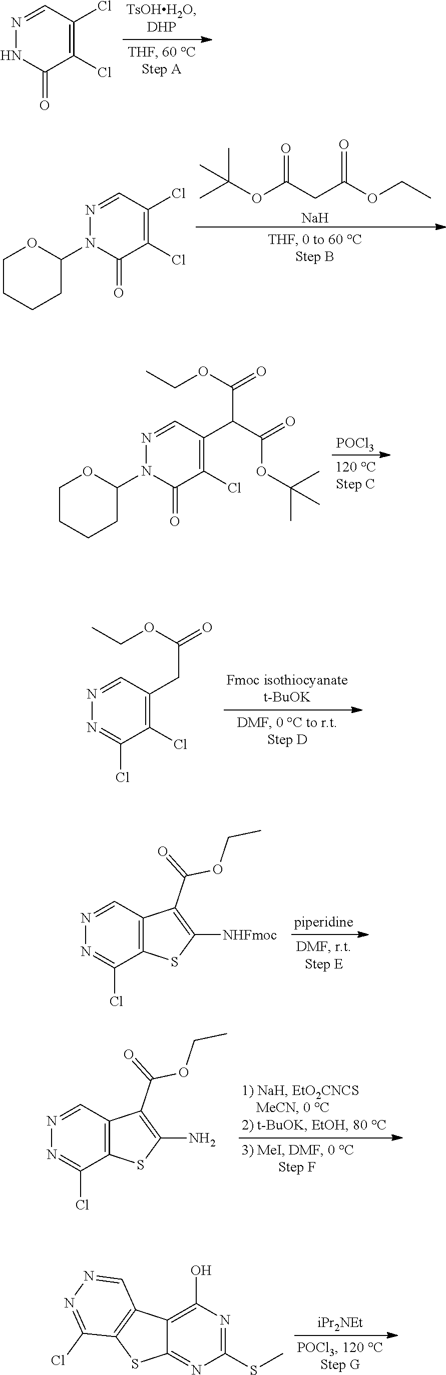

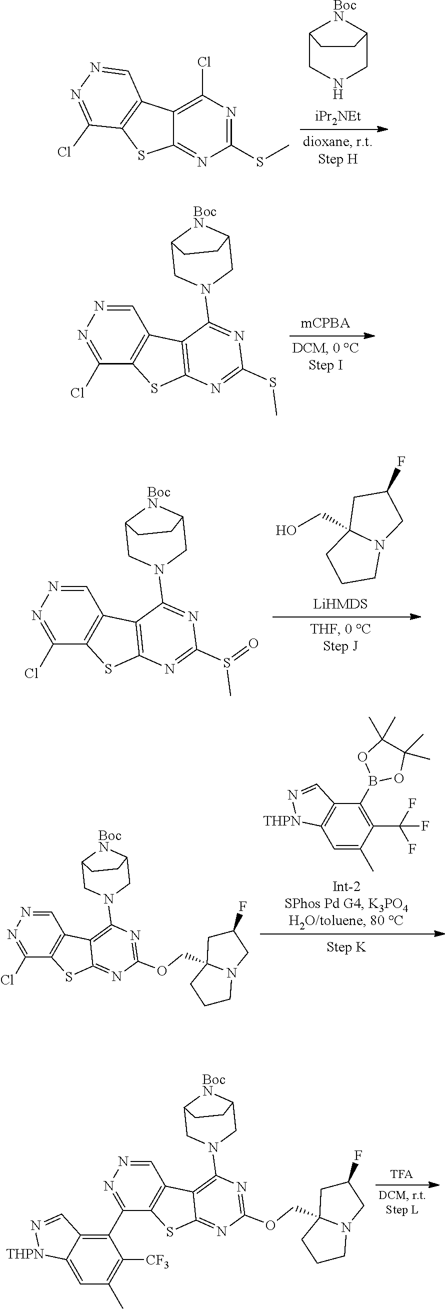

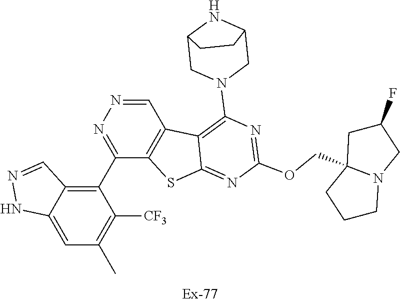 [SMILES: Cc1cc2[nH]ncc2c(-c2nncc3c2sc2nc(OC[C@@]45CCCN4C[C@H](F)C5)nc(N4CC5CCC(C4)N5)c23)c1C(F)(F)F]

### Step A: 4,5-dichloro-2-(tetrahydro-2H-pyran-2-yl)pyridazin-3(2H)-one

A round bottom flask with a stir bar was charged with 4,5-dichloropyridazin-3(2H)-one (5.0 g, 30 mmol), p-toluenesulfonic acid monohydrate (1.2 g, 6.1 mmol), 3,4-dihydro-2H-pyran (4.1 mL, 46 mmol), and THF (30 mL). The reaction mixture was stirred at 60° C. for 11 h. The reaction mixture was cooled to r.t. and concentrated under reduced pressure. To the residue was added EtOAc, and the mixture was washed with water (50 mL) and brine (50 mL), dried over sodium sulfate, filtered, and concentrated under reduced pressure. The residue was purified by silica gel chromatography (10% EtOAc in hexanes) to give 4,5-dichloro-2-(tetrahydro-2H-pyran-2-yl)pyridazin-3(2H)-one. MS (ESI): m/z (M+H)⁺ 249, 251. ¹H NMR (400 MHz, CDCl₃) δ 7.84 (s, 1H), 4.15-4.11 (m, 1H), 3.78-3.72 (m, 2H), 2.08-2.00 (m, 2H), 1.78-1.67 (m, 4H).

### Step B: 1-(tert-butyl) 3-ethyl 2-(5-chloro-6-oxo-1-(tetrahydro-2H-pyran-2-yl)-1,6-dihydropyridazin-4-yl)malonate

A round bottom flask with a stir bar was charged with NaH (1.40 g, 34.9 mmol, 60 wt % in mineral oil) and THF (30 mL). The reaction mixture was cooled to 0° C. To this mixture was added tert-butyl ethyl malonate (3.97 mL, 21 mmol) at 0° C. After stirring this mixture at 0° C. for 10 min, a solution of 4,5-dichloro-2-(tetrahydro-2H-pyran-2-yl)pyridazin-3(2H)-one (4.35 g, 17.5 mmol) in THF (20 mL) was added. The reaction mixture was stirred at 0° C. for 5 min, and then stirred at 60° C. for 15 min. The reaction mixture was cooled to r.t. and quenched by the addition of H₂O and 2 M aq. HCl (35 mL). The reaction mixture was extracted with EtOAc (2×50 mL). The combined organic layers were washed with brine (20 mL), dried over sodium sulfate, filtered, and concentrated under reduced pressure. The residue was purified by silica gel chromatography (30% EtOAc in hexanes) to give 1-(tert-butyl) 3-ethyl 2-(5-chloro-6-oxo-1-(tetrahydro-2H-pyran-2-yl)-1,6-dihydropyridazin-4-yl)malonate. MS (ESI): m/z (M+H)⁺ 401, 403.

### Step C: Ethyl 2-(5,6-dichloropyridazin-4-yl)acetate

A round bottom flask with a stir bar was charged with 1-(tert-butyl) 3-ethyl 2-(5-chloro-6-oxo-1-(tetrahydro-2H-pyran-2-yl)-1,6-dihydropyridazin-4-yl)malonate (7.79 g, 17.8 mmol) and phosphoryl chloride (18 mL). The reaction mixture was stirred at 120° C. for 20 min. The reaction mixture was cooled to r.t. and evaporated. To this residue was added toluene, and the mixture was evaporated. EtOAc was added, and the organic layer was washed with water, sat. aq. NaHCO₃, and brine (20 mL each). The organic layer was dried over sodium sulfate, filtered, and concentrated under reduced pressure. The residue was purified by silica gel chromatography (0 to 50% EtOAc in hexanes) to give ethyl 2-(5,6-dichloropyridazin-4-yl)acetate. MS (ESI): m/z (M+H)⁺ 235, 237.

### Step D: Ethyl 2-((((9H-fluoren-9-yl)methoxy)carbonyl)amino)-7-chlorothieno[2,3-d]pyridazine-3-carboxylate

A round bottom flask with a stir bar was charged with ethyl 2-(5,6-dichloropyridazin-4-yl)acetate (649 mg, 2.76 mmol) and DMF (9 mL). The mixture was cooled to 0° C. and potassium tert-butoxide (326 mg, 2.9 mmol) was added portionwise. The reaction mixture was stirred at 0° C. for 5 min, and then Fmoc isothiocyanate (816 mg, 2.9 mmol) was added at 0° C. The reaction mixture was stirred at r.t. for 3 min. H₂O was added slowly with stirring. The resulting precipitate was collected by filtration, rinsed with H₂O and hexane, and dried under reduced pressure to give ethyl 2-((((9H-fluoren-9-yl)methoxy)carbonyl)amino)-7-chlorothieno[2,3-d]pyridazine-3-carboxylate. MS (ESI): m/z (M+H)⁺ 480, 482.

### Step E: Ethyl 2-amino-7-chlorothieno[2,3-d]pyridazine-3-carboxylate

A round bottom flask with a stir bar was charged with ethyl 2-((((9H-fluoren-9-yl)methoxy)carbonyl)amino)-7-chlorothieno[2,3-d]pyridazine-3-carboxylate (100 mg, 0.21 mmol), piperidine (45 μL, 0.46 mmol), and DMF (0.7 mL). The reaction mixture was stirred at r.t. for 15 min. H₂O was added slowly with stirring. The resulting precipitate was collected by filtration, rinsed with H₂O and hexane, and dried under reduced pressure to give ethyl 2-amino-7-chlorothieno[2,3-d]pyridazine-3-carboxylate. MS (ESI): m/z (M+H)+258, 260.

### Step F: 8-chloro-2-(methylthio)pyrimido[5′,4′:4,5]thieno[2,3-d]pyridazin-4-ol

A round bottom flask with a stir bar was charged with ethyl 2-amino-7-chlorothieno[2,3-d]pyridazine-3-carboxylate (115 mg, 0.450 mmol) and MeCN (9.0 mL). The reaction mixture was cooled to 0° C. To this mixture was added NaH (21.0 mg, 0.530 mmol, 60 wt % in mineral oil) at 0° C. After stirring the mixture at 0° C. for 5 min, ethoxycarbonyl isothiocyanate (78.7 μL, 0.667 mmol) was added to the reaction mixture at 0° C. After the reaction mixture was stirred at 0° C. for 90 min, MeOH was added and the reaction mixture was evaporated. To the residue was added potassium tert-butoxide (150 mg, 1.3 mmol) and EtOH (9.0 mL), and the reaction mixture was stirred at 80° C. for 1 h. The reaction was cooled to room temperature and concentrated. To the residue was added DMF (4.4 mL). After the reaction mixture was cooled to 0° C., MeI (31 μL, 0.49 mmol) was added. The reaction mixture was stirred at 0° C. for 5 min. H₂O and 2M HCl (1.4 mL) were added slowly with stirring. The resulting precipitate was collected by filtration, rinsed with H₂O and hexane, and dried under reduced pressure to give 8-chloro-2-(methylthio)pyrimido[5′,4′:4,5]thieno[2,3-d]pyridazin-4-ol. MS (ESI): m/z (M+H)⁺ 285, 287.

### Step G: 4,8-dichloro-2-(methylthio)pyrimido[5′,4′:4,5]thieno[2,3-d]pyridazine

A 20 mL round bottom flask with a stir bar was charged with 8-chloro-2-(methylthio)pyrimido[5′,4′:4,5]thieno[2,3-d]pyridazin-4-ol (80 mg, 0.28 mmol), phosphoryl chloride (0.94 mL) and N,N-diisopropylethylamine (0.096 mL, 0.56 mmol). The reaction mixture was stirred at 120° C. for 5 min. The reaction mixture was cooled to r.t. and evaporated. To this residue was added water and CHCl₃. The layers were separated and the aqueous phase was extracted with CHCl₃ (3×10 mL). The combined organic layer was washed with brine (10 mL), dried over sodium sulfate, filtered, and concentrated under reduced pressure to give 4,8-dichloro-2-(methylthio)pyrimido[5′,4′:4,5]thieno[2,3-d]pyridazine. MS (ESI): m/z (M+H)⁺ 303, 305. ¹H NMR (400 MHz, CDCl₃) δ 10.13 (s, 1H), 2.72 (s, 3H).

### Step H: Tert-butyl (1R,5S)-3-(8-chloro-2-(methylthio)pyrimido[5′,4′:4,5]thieno[2,3-d]pyridazin-4-yl)-3,8-diazabicyclo[3.2.1]octane-8-carboxylate

To a stirred mixture of 4,8-dichloro-2-(methylthio)pyrimido[5′,4′:4,5]thieno[2,3-d]pyridazine (100 mg, 0.330 mmol) and N,N-diisopropylethylamine (0.113 ml, 0.66 mmol) in dioxane (3.3 mL) was added tert-butyl 3,8-diazabicyclo[3.2.1]octane-8-carboxylate (77.0 mg, 0.363 mmol) at room temperature. After being stirred for 15 min, water and EtOAc was added to the mixture. The organic phase was washed with water and brine, dried over sodium sulfate, and concentrated. The residue was purified via silica gel chromatography (0 to 30% EtOAc in hexane) to give tert-butyl (1R,5S)-3-(8-chloro-2-(methylthio)pyrimido[5′,4′:4,5]thieno[2,3-d]pyridazin-4-yl)-3,8-diazabicyclo[3.2.1]octane-8-carboxylate. MS (ESI): m/z (M+H)⁺ 479. ¹H NMR (400 MHz, CDCl₃) δ=9.34 (s, 1H), 4.49-4.10 (m, 4H), 3.77-3.64 (m, 2H), 2.66 (s, 3H), 1.98-1.87 (m, 2H), 1.76-1.64 (m, 2H), 1.55 (s, 9H).

### Step I: Tert-butyl (1R,5S)-3-(8-chloro-2-(methylsulfinyl)pyrimido[5′,4′:4,5]thieno[2,3-d]pyridazin-4-yl)-3,8-diazabicyclo[3.2.1]octane-8-carboxylate

To a stirred solution of tert-butyl (1R,5S)-3-(8-chloro-2-(methylthio)pyrimido[5′,4′:4,5]thieno[2,3-d]pyridazin-4-yl)-3,8-diazabicyclo[3.2.1]octane-8-carboxylate (75 mg, 0.16 mmol) in DCM (1.6 mL) was added m-chloroperoxybenzoic acid (36 mg, 0.16 mmol, 75 wt %) at 0° C. After 30 min, sat. aq. NaHCO₃ was added to the mixture and the mixture was extracted with EtOAc. The organic phase was washed with brine, dried over sodium sulfate, filtered, and concentrated to give tert-butyl (1R,5S)-3-(8-chloro-2-(methylsulfinyl)pyrimido[5′,4′:4,5]thieno[2,3-d]pyridazin-4-yl)-3,8-diazabicyclo[3.2.1]octane-8-carboxylate which was used in the next step without further purification. MS (ESI): m/z (M+H)⁺ 495.

### Step J: Tert-butyl (1R,5S)-3-(8-chloro-2-(((2R,7aS)-2-fluorotetrahydro-1H-pyrrolizin-7a(5H)-yl)methoxy)pyrimido[5′,4′:4,5]thieno[2,3-d]pyridazin-4-yl)-3,8-diazabicyclo[3.2.1]octane-8-carboxylate

To a mixture of ((2R,7aS)-2-fluorotetrahydro-1H-pyrrolizin-7a(5H)-yl)methanol (37 mg, 0.235 mmol) in THF (1.6 mL) was added LiHMDS (5.0 mL, 5.0 mmol, 1 M in THF) at 0° C. and stirred at 0° C. for 20 min. Then tert-butyl (1R,5S)-3-(8-chloro-2-(methylsulfinyl)pyrimido[5′,4′:4,5]thieno[2,3-d]pyridazin-4-yl)-3,8-diazabicyclo[3.2.1]octane-8-carboxylate (87 mg, 0.177 mmol) in THF (1.6 mL) was added and stirred for another 30 min at 0° C. H₂O was added and the mixture was extracted with EtOAc (3 times). The combined organic phases were dried over sodium sulfate, filtered, and concentrated. The residue was purified via silica gel chromatography (0 to 3% MeOH in CHCl₃) to give tert-butyl (1R,5S)-3-(8-chloro-2-(((2R,7aS)-2-fluorotetrahydro-1H-pyrrolizin-7a(5H)-yl)methoxy)pyrimido[5′,4′:4,5]thieno[2,3-d]pyridazin-4-yl)-3,8-diazabicyclo[3.2.1]octane-8-carboxylate. MS (ESI): m/z (M+H)⁺ 590. ¹H NMR (400 MHz, CDCl₃) δ=9.32 (s, 1H), 5.42-5.20 (m, 1H), 4.50-4.08 (m, 6H), 3.77-3.62 (m, 2H), 3.34-3.26 (m, 2H), 3.26-3.12 (m, 1H), 3.07-2.96 (m, 1H), 2.39-2.24 (m, 1H), 2.24-2.10 (m, 2H), 2.05-1.82 (m, 5H), 1.76-1.66 (m, 2H), 1.56 (s, 9H).

### Step K: Tert-butyl (1R,5S)-3-(2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)pyrimido[5′,4′:4,5]thieno[2,3-d]pyridazin-4-yl)-3,8-diazabicyclo[3.2.1]octane-8-carboxylate

A mixture of tert-butyl (1R,5S)-3-(8-chloro-2-(((2R,7aS)-2-fluorotetrahydro-1H-pyrrolizin-7a(5H)-yl)methoxy)pyrimido[5′,4′:4,5]thieno[2,3-d]pyridazin-4-yl)-3,8-diazabicyclo[3.2.1]octane-8-carboxylate (23 mg, 0.039 mmol), 6-methyl-1-(tetrahydro-2H-pyran-2-yl)-4-(4,4,5,5-tetramethyl-1,3,2-dioxaborolan-2-yl)-5-(trifluoromethyl)-1H-indazole (Int-2) (27.2 mg, 0.066 mmol), SPhos Pd G4 (15.5 mg, 0.019 mmol), and K₃PO₄ (0.2 mL, 0.20 mmol, 1M in H₂O) in toluene (0.80 mL) was degassed and charged with nitrogen 3 times. The mixture was heated to 80° C. and stirred under nitrogen atmosphere for 3 h. After cooling, the mixture was partitioned between EtOAc and water. The organic phase was washed with brine, dried over sodium sulfate, filtered, and concentrated. The crude residue was purified via silica gel chromatography (0 to 7% MeOH in CHCl₃) to give tert-butyl (1R,5S)-3-(2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)pyrimido[5′,4′:4,5]thieno[2,3-d]pyridazin-4-yl)-3,8-diazabicyclo[3.2.1]octane-8-carboxylate. MS (ESI): m/z (M+H)⁺ 838.

### Step L: 4-((1R,5S)-3,8-diazabicyclo[3.2.1]octan-3-yl)-2-(((2R,7aS)-2-fluorotetrahydro-1H-pyrrolizin-7a(5H)-yl)methoxy)-8-(6-methyl-5-(trifluoromethyl)-1H-indazol-4-yl)pyrimido[5′,4′:4,5]thieno[2,3-d]pyridazine (Ex-77)

To a stirred solution of tert-butyl (1R,5S)-3-(2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)pyrimido[5′,4′:4,5]thieno[2,3-d]pyridazin-4-yl)-3,8-diazabicyclo[3.2.1]octane-8-carboxylate (28 mg, 0.033 mmol) in DCM (0.8 mL) was added TFA (200 μL). After 30 min, the reaction was concentrated in vacuo and the residue was purified by reverse phase HPLC (MeCN/H₂O w/HCOOH modifier) to obtain 4-((1R,5S)-3,8-diazabicyclo[3.2.1]octan-3-yl)-2-(((2R,7aS)-2-fluorotetrahydro-1H-pyrrolizin-7a(5H)-yl)methoxy)-8-(6-methyl-5-(trifluoromethyl)-1H-indazol-4-yl)pyrimido[5′,4′:4,5]thieno[2,3-d]pyridazine (Ex-77). MS (ESI): m/z (M+H)⁺ 655. ¹H NMR (400 MHz, CDCl₃) δ 9.49 (s, 1H), 7.61 (s, 1H), 7.48 (s, 1H), 5.34-5.20 (m, 1H), 4.31-4.28 (m, 2H), 4.22 (d, J=10.16 Hz, 1H), 4.10 (d, J=10.16 Hz, 1H), 3.70-3.65 (m, 4H), 3.28-3.15 (m, 3H), 3.02-2.96 (m, 1H), 2.75-2.73 (brs, 3H), 2.34-2.28 (m, 1H), 2.24-2.04 (m, 3H), 1.96-1.75 (m, 6H).

### Example 78: 4-((1R,5S)-3,8-diazabicyclo[3.2.1]octan-3-yl)-2-(((2R,7aS)-2-fluorotetrahydro-1H-pyrrolizin-7a(5H)-yl)methoxy)-8-(6-methyl-5-(trifluoromethyl)-1H-indazol-4-yl)pyrido[4′,3′:4,5]furo[2,3-d]pyrimidine (Ex-78)

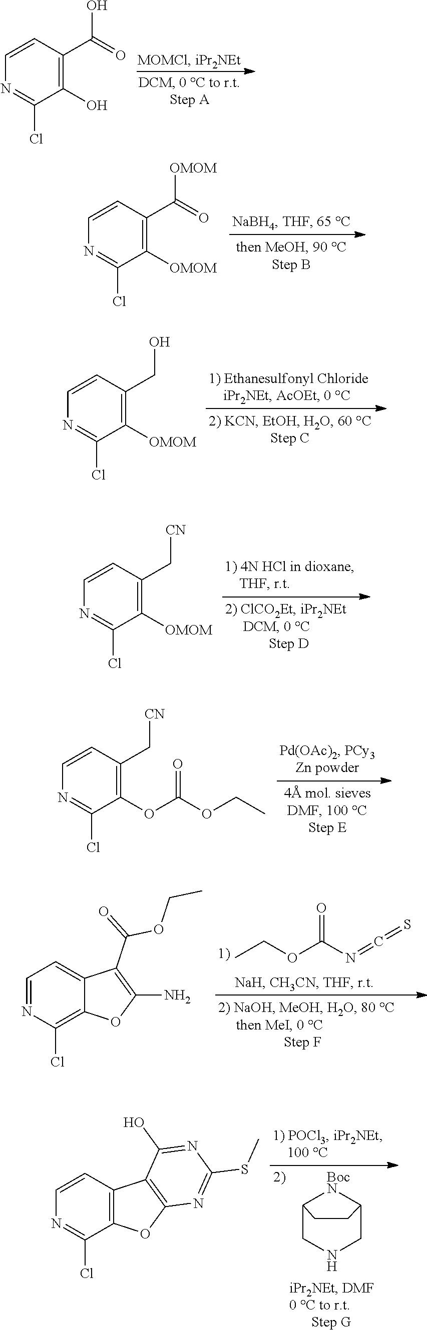

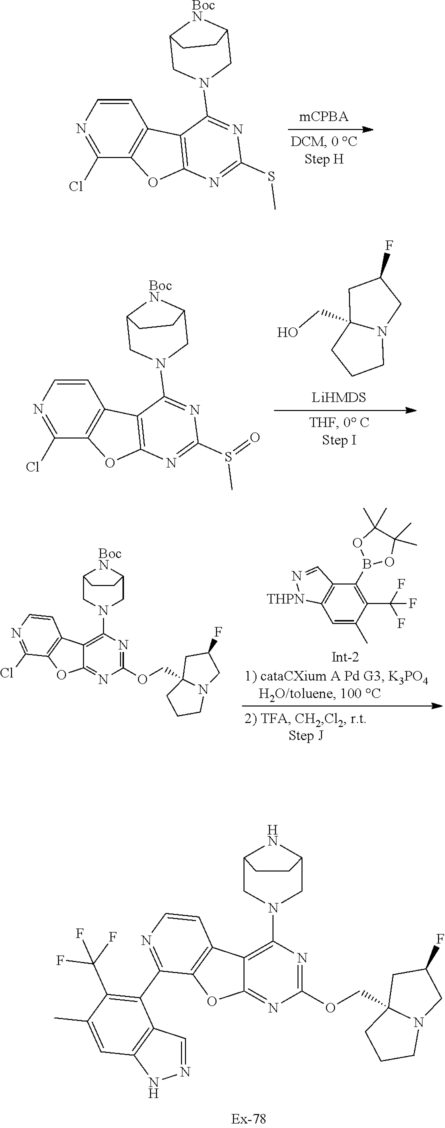

### Step A: Methoxymethyl 2-chloro-3-(methoxymethoxy)isonicotinate

To a stirred mixture of 2-chloro-3-hydroxyisonicotinic acid (5.0 g, 29 mmol) and N,N-diisopropylethylamine (25 mL, 140 mmol) in DCM (150 mL) was added chloro(methoxy)methane (7.7 mL, 100 mmol) at 0° C. The reaction was allowed to warm to r.t. over 3 h. After 3 h, the reaction was quenched with water and the mixture was extracted with chloroform. The organic phase was washed with brine, dried over sodium sulfate, filtered, and concentrated. The crude residue was purified via silica gel chromatography (15% to 30% EtOAc in hexane) to give methoxymethyl 2-chloro-3-(methoxymethoxy)isonicotinate. MS (ESI): m/z (M+H)⁺ 262. ¹H NMR (400 MHz, CDCl₃) δ 8.29 (d, J=5.0 Hz, 1H), 7.60 (d, J=4.9 Hz, 1H), 5.51 (s, 2H), 5.23 (s, 2H), 3.65 (s, 3H), 3.59 (s, 3H).

### Step B: (2-chloro-3-(methoxymethoxy)pyridin-4-yl)methanol

To a stirred solution of methoxymethyl 2-chloro-3-(methoxymethoxy)isonicotinate (5.7 g, 22 mmol) in THF (109 mL) was added sodium borohydride (7.02 g, 185 mmol) at 65° C. portionwise. MeOH (110 ml) was added dropwise, and the mixture was warmed to 90° C. and stirred for 0.5 h. After cooling to room temperature, the reaction was quenched with sat. aq. ammonium chloride and concentrated in vacuo. To the residue was added EtOAc and the mixture was washed with brine, dried over sodium sulfate, filtered, and concentrated to give (2-chloro-3-(methoxymethoxy)pyridin-4-yl)methanol. MS (ESI): m/z (M+H)⁺ 204. ¹H NMR (400 MHz, CDCl₃) δ 8.22 (d, J=4.8 Hz, 1H), 7.36 (d, J=4.9 Hz, 1H), 5.18 (s, 2H), 4.75 (s, 2H), 3.67 (s, 3H), 2.81 (br s, 1H).

### Step C: 2-(2-chloro-3-(methoxymethoxy)pyridin-4-yl)acetonitrile

To a stirred solution of (2-chloro-3-(methoxymethoxy)pyridin-4-yl)methanol (3.53 g, 17.3 mmol) and N,N-diisopropylethylamine (5.93 mmol, 34.7 mmol) in EtOAc (87 mL) was added ethanesulfonyl chloride (2.46 mmol, 26 mmol) at 0° C. The mixture was stirred at 0° C. for 0.5 h. Then the mixture was diluted with EtOAc and quenched with water. The organic phase was washed with water and brine, dried over sodium sulfate, filtered, and concentrated to give a corresponding ethanesulfonyl compound which was used directly in the next step without further purification. A solution of the crude ethanesulfonyl compound in ethanol (43 ml) was added to a stirred solution of potassium cyanide (2.26 g, 34.7 mmol) in water (43 ml) at 60° C. Then the mixture was stirred at 60° C. for 1.5 h. After cooling, EtOAc was added, and the mixture was washed with brine, dried over sodium sulfate, filtered, and concentrated. The crude residue was purified via silica gel chromatography (15 to 48% EtOAc in hexane) to give 2-(2-chloro-3-(methoxymethoxy)pyridin-4-yl)acetonitrile. MS (ESI): m/z (M+H)⁺ 213. ¹H NMR (400 MHz, CDCl₃) δ 8.26 (d, J=4.9 Hz, 1H), 7.43 (d, J=4.9 Hz, 1H), 5.20 (s, 2H), 3.96 (s, 2H), 3.63 (s, 3H).

### Step D: 2-chloro-4-(cyanomethyl)pyridin-3-yl ethyl carbonate

To a stirred suspension of 2-(2-chloro-3-(methoxymethoxy)pyridin-4-yl)acetonitrile (1.2 g, 5.6 mmol) in THF (2.5 mL) was added HCl (5.0 ml, 4N in dioxane) at r.t. After being stirred for 1 h, the mixture was concentrated in vacuo. DCM (20 mL) and N,N-diisopropylethylamine (4.8 mL, 28 mmol) were added, and the mixture was cooled to 0° C. Ethyl chloroformate (0.65 mL, 6.8 mmol) was added, and the reaction was stirred at 0° C. for 0.5 h. The reaction was quenched with water and the mixture was extracted with chloroform. The organic phase was washed with brine, dried over sodium sulfate, filtered, and concentrated. The crude residue was purified via silica gel chromatography (15 to 55% EtOAc in hexane) to give 2-chloro-4-(cyanomethyl)pyridin-3-yl ethyl carbonate. MS (ESI): m/z (M+H)⁺ 241. ¹H NMR (400 MHz, CDCl₃) δ 8.39 (d, J=5.0 Hz, 1H), 7.48 (d, J=5.0 Hz, 1H), 4.43 (q, J=7.1 Hz, 2H), 3.81 (s, 2H), 1.45 (t, J=7.1 Hz, 3H).

### Step E: Ethyl 2-amino-7-chlorofuro[2,3-c]pyridine-3-carboxylate

A mixture of palladium acetate (75 mg, 0.32 mmol), tricyclohexylphosphine (190 mg, 0.67 mmol), zinc (45 mg, 0.67 mmol), and powdered 4 Å molecular sieves (350 mg) in DMF (24 mL) was degassed 3 times and stirred at r.t. for 15 min. Then a solution of 2-chloro-4-(cyanomethyl)pyridin-3-yl ethyl carbonate (800 mg, 3.3 mmol) in DMF (12 mL) was added, and the mixture was heated at 100° C. for 9 h. After cooling to room temperature, the mixture was filtered through a CELITE® pad and the filtrate was partitioned between EtOAc and water. The organic phase was washed with water and brine, dried over sodium sulfate, filtered, and concentrated. The crude residue was purified via silica gel chromatography (20 to 60% EtOAc in hexane) to give ethyl 2-amino-7-chlorofuro[2,3-c]pyridine-3-carboxylate. MS (ESI): m/z (M+H)⁺ 241. ¹H NMR (400 MHz, CDCl₃) δ 8.16 (d, J=5.1 Hz, 1H), 7.52 (d, J=5.1 Hz, 1H), 6.34 (br s, 2H), 4.41 (q, J=7.1 Hz, 2H), 1.46 (t, J=7.1 Hz, 3H).

### Step F: 8-chloro-2-(methylthio)pyrido[4′,3′:4,5]furo[2,3-d]pyrimidin-4-ol

To a stirred solution of ethyl 2-amino-7-chlorofuro[2,3-c]pyridine-3-carboxylate (80 mg, 0.33 mmol) and ethoxycarbonyl isothiocyanate (58 μL, 0.50 mmol) in acetonitrile (3 mL) and THF (1 mL) was added sodium hydride (17 mg, 0.43 mmol, 60 wt % in mineral oil) at r.t. After being stirred for 0.5 h, the volatile materials were removed by evaporation. To the residue was added EtOAc and the resulting precipitate was collected by filtration to give a thiourea compound, which was used directly in the next step without further purification. A mixture of the thiourea compound in 1 M aq. NaOH (1.6 mL), water (0.8 mL) and methanol (1.6 mL) was stirred at 80° C. for 1.5 h. Then the mixture was cooled to 0° C. and methyl iodide (25 μL, 0.40 mmol) was added to the mixture. The reaction mixture was stirred for 0.5 h at 0° C., and then acidified to pH 3 by dilute hydrochloric acid. The resulting precipitate was collected by filtration to give 8-chloro-2-(methylthio)pyrido[4′,3′:4,5]furo[2,3-d]pyrimidin-4-ol. MS (ESI): m/z (M+H)⁺ 268. ¹H NMR (400 MHz, DMSO-d₆) δ 13.52 (br s, 1H), 8.35 (d, J=5.00 Hz, 1H), 7.82 (d, J=5.00 Hz, 1H), 2.64 (s, 3H).

### Step G: Tert-butyl (1R,5S)-3-(8-chloro-2-(methylthio)pyrido[4′,3′:4,5]furo[2,3-d]pyrimidin-4-yl)-3,8-diazabicyclo[3.2.1]octane-8-carboxylate

A mixture of 8-chloro-2-(methylthio)pyrido[4′,3′:4,5]furo[2,3-d]pyrimidin-4-ol (49 mg, 0.43 mmol), phosphorus oxychloride (1.00 mL, 10.7 mmol) and N,N-diisopropylethylamine (63 ul, 0.37 mmol) was stirred at 100° C. for 1.5 h. The volatile materials were removed by evaporation and the residue was partitioned between EtOAc and sat. aq. sodium bicarbonate. The organic phase was washed with brine, dried over sodium sulfate, filtered, and concentrated to give desired the crude, which was used directly in the next step without further purification. To a solution of the crude material and N,N-diisopropylethylamine (315 mL, 1.83 mmol) in DMF (2 mL) was added tert-butyl (1R,5S)-3,8-diazabicyclo[3.2.1]octane-8-carboxylate (58 mg, 0.28 mmoL) at 0° C. The reaction mixture was allowed to stir to ambient temperature for 0.5 h then partitioned between EtOAc and water. The organic phase was washed with water and brine, dried over sodium sulfate, filtered, and concentrated. The crude residue was purified via silica gel chromatography chromatography (20 to 60% EtOAc in hexane) to give tert-butyl (1R,5S)-3-(8-chloro-2-(methylthio)pyrido[4′,3′:4,5]furo[2,3-d]pyrimidin-4-yl)-3,8-diazabicyclo[3.2.1]octane-8-carboxylate. MS (ESI): m/z (M+H)⁺ 462. ¹H NMR (400 MHz, CDCl₃) δ=8.34 (d, J=5.4 Hz, 1H), 7.49 (d, J=5.4 Hz, 1H), 4.74-4.26 (m, 4H), 3.89-3.36 (m, 2H), 2.64 (s, 3H), 2.04-1.91 (m, 2H), 1.82-1.62 (m, 2H), 1.55 (s, 9H).

### Step H: Tert-butyl (1R,5S)-3-(8-chloro-2-(methylsulfinyl)pyrido[4′,3′:4,5]furo[2,3-d]pyrimidin-4-yl)-3,8-diazabicyclo[3.2.1]octane-8-carboxylate

To a stirred solution of tert-butyl (1R,5S)-3-(8-chloro-2-(methylthio)pyrido[4′,3′:4,5]furo[2,3-d]pyrimidin-4-yl)-3,8-diazabicyclo[3.2.1]octane-8-carboxylate (77 mg, 0.17 mmol) in DCM (5 ml) was added m-chloroperoxybenzoic acid (38 mg, 0.17 mmol, 75 wt %) at 0° C. After 30 min sat. aq. NaHCO₃ was added and the mixture was extracted with CHCl₃. The organic extract was washed with brine, dried over sodium sulfate, filtered, and concentrated to give tert-butyl (1R,5S)-3-(8-chloro-2-(methylsulfinyl)pyrido[4′,3′:4,5]furo[2,3-d]pyrimidin-4-yl)-3,8-diazabicyclo[3.2.1]octane-8-carboxylate which was used in the next step without further purification. MS (ESI): m/z (M+H)⁺ 478.

### Step I: Tert-butyl (1R,5S)-3-(8-chloro-2-(((2R,7aS)-2-fluorotetrahydro-1H-pyrrolizin-7a(5H)-yl)methoxy)pyrido[4′3′:4,5]furo[2,3-d]pyrimidin-4-yl)-3,8-diazabicyclo[3.2.1]octane-8-carboxylate

To a stirred solution of ((2R,7aS)-2-fluorotetrahydro-1H-pyrrolizin-7a(5H)-yl)methanol (40 mg, 0.25 mmol) in THF (8 mL) was added LiHMDS (420 μL, 0.42 mmol, 1M solution in THF) at 0° C. and stirred at 0° C. for 15 min. To the mixture was added tert-butyl (1R,5S)-3-(8-chloro-2-(methylsulfinyl)pyrido[4′,3′:4,5]furo[2,3-d]pyrimidin-4-yl)-3,8-diazabicyclo[3.2.1]octane-8-carboxylate in THF (2 mL) dropwise via syringe. After the mixture was allowed to stir at 0° C. for 30 min, the mixture was partitioned between EtOAc and water and separated. The organic phase was dried over sodium sulfate, filtered, and concentrated. The crude residue was purified via NH-silica gel chromatography (40 to 80% EtOAc in Hexane) to give tert-butyl (1R,5S)-3-(8-chloro-2-(((2R,7aS)-2-fluorotetrahydro-1H-pyrrolizin-7a(5H)-yl)methoxy)pyrido[4′,3′:4,5]furo[2,3-d]pyrimidin-4-yl)-3,8-diazabicyclo[3.2.1]octane-8-carboxylate. MS (ESI): m/z (M+H)⁺ 573. ¹H NMR (400 MHz, CDCl₃) δ=8.31 (d, J=5.4 Hz, 1H), 7.47 (d, J=5.4 Hz, 1H), 5.49-5.16 (m, 1H), 4.65-4.29 (m, 4H), 4.26-4.20 (m, 1H), 4.17-4.10 (m, 1H), 3.78-3.41 (m, 2H), 3.33-3.24 (m, 2H), 3.24-3.14 (m, 1H), 3.07-2.94 (m, 1H), 2.27 (d, J=4.3 Hz, 1H), 2.24-2.16 (m, 1H), 2.16-2.10 (m, 1H), 2.03-1.85 (m, 5H), 1.80-1.67 (m, 2H), 1.55 (s, 9H).

### Step J: 4-((1R,5S)-3,8-diazabicyclo[3.2.1]octan-3-yl)-2-(((2R,7aS)-2-fluorotetrahydro-1H-pyrrolizin-7a(5H)-yl)methoxy)-8-(6-methyl-5-(trifluoromethyl)-1H-indazol-4-yl)pyrido[4′,3′:4,5]furo[2,3-d]pyrimidine (Ex-78)

A mixture of tert-butyl (1R,5S)-3-(8-chloro-2-(((2R,7aS)-2-fluorotetrahydro-1H-pyrrolizin-7a(5H)-yl)methoxy)pyrido[4′,3′:4,5]furo[2,3-d]pyrimidin-4-yl)-3,8-diazabicyclo[3.2.1]octane-8-carboxylate (40 mg, 0.070 mmol), 6-methyl-1-(tetrahydro-2H-pyran-2-yl)-4-(4,4,5,5-tetramethyl-1,3,2-dioxaborolan-2-yl)-5-(trifluoromethyl)-1H-indazole (Int-2) (43 mg, 0.10 mmol), cataCXium A Pd G3 (25 mg, 0.035 mmol), and K₃PO₄ (0.35 mL, 0.35 mmol, 1M in H₂O) in toluene (1.2 mL) was evacuated and charged with N₂ 3 times. The mixture was stirred at 100° C. under nitrogen atmosphere for 5 h. After cooling to room temperature, the mixture was partitioned between EtOAc and water. The organic phase was washed with brine, dried over sodium sulfate, filtered, and concentrated. To the crude mixture was added DCM (1.2 mL) and TFA (500 μL) successively and the reaction was stirred at room temperature for 1 hr. The reaction was concentrated in vacuo, and the residue was partitioned between sat. aq. NaHCO₃ and CHCl₃. The organic phase was washed with brine, dried over sodium sulfate, filtered, and concentrated. The crude residue was purified via reverse phase HPLC (MeCN/H₂O w/HCOOH modifier) to give 4-((1R,5S)-3,8-diazabicyclo[3.2.1]octan-3-yl)-2-(((2R,7aS)-2-fluorotetrahydro-1H-pyrrolizin-7a(5H)-yl)methoxy)-8-(6-methyl-5-(trifluoromethyl)-1H-indazol-4-yl)pyrido[4′,3′:4,5]furo[2,3-d]pyrimidine (Ex-78). MS (ESI): m/z (M+H)⁺ 637. ¹H NMR (400 MHz, CDCl₃) δ=10.40-9.96 (m, 1H), 8.64 (d, J=5.4 Hz, 1H), 7.63 (d, J=5.4 Hz, 1H), 7.56-7.50 (m, 2H), 5.37-5.17 (m, 1H), 4.61-4.50 (m, 2H), 4.22-4.03 (m, 2H), 3.74 (br s, 2H), 3.64-3.54 (m, 2H), 3.31-3.12 (m, 3H), 3.02-2.92 (m, 1H), 2.73 (s, 3H), 2.26-2.21 (m, 1H), 2.18-2.12 (m, 1H), 2.12-2.04 (m, 1H), 1.99-1.85 (m, 3H), 1.85-1.74 (m, 4H).

### Example 79: 4-((1R,5S)-3,8-diazabicyclo[3.2.1]octan-3-yl)-8-(5-(1-fluorocyclopropyl)-6-(trifluoromethyl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidine (Ex-79)

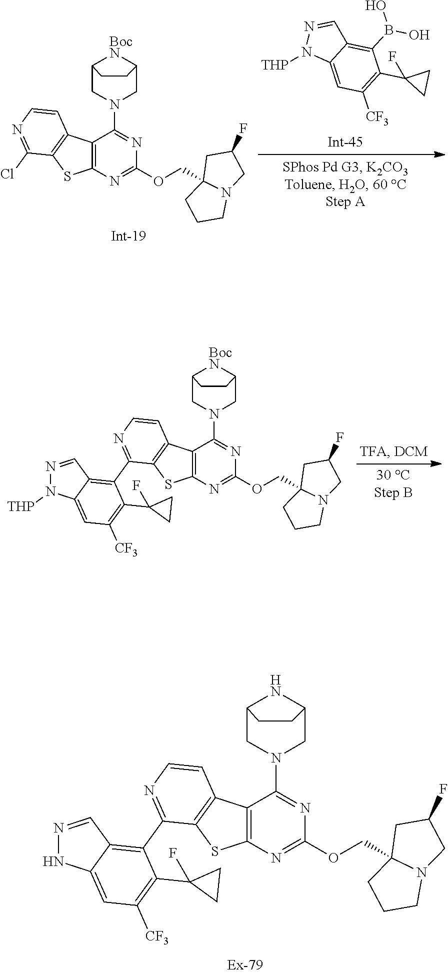

### Step A: tert-butyl (1R,5S)-3-(8-(5-(1-fluorocyclopropyl)-1-(tetrahydro-2H-pyran-2-yl)-6-(trifluoromethyl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate

To a solution of tert-butyl (1R,5S)-3-(8-chloro-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate (Int-19) (100 mg, 0.170 mmol) in toluene (2.5 mL) was added (2-dicyclohexylphosphino-2′,6′-dimethoxybiphenyl) [2-(2′-amino-1,1′-biphenyl)]palladium(II) methanesulfonate (132 mg, 0.170 mmol), (5-(1-fluorocyclopropyl)-1-(tetrahydro-2H-pyran-2-yl)-6-(trifluoromethyl)-1H-indazol-4-yl)boronic acid (Int-45) (95 mg, 0.255 mmol) and K₂CO₃ (0.424 mL, 0.849 mmol). The reaction was stirred at 60° C. for 13 h. The reaction was diluted with water (10 mL), extracted with EtOAc (10 mL×2). The combined organic layer was washed with brine (20 mL), dried over Na₂SO₄, filtered, concentrated, and the residue was purified by preparative TLC (DCM:MeOH=20:1) to give tert-butyl (1R,5S)-3-(8-(5-(1-fluorocyclopropyl)-1-(tetrahydro-2H-pyran-2-yl)-6-(trifluoromethyl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate. MS (ESI) [M+H]⁺: m/z 881.

### Step B: 4-((1R,5S)-3,8-diazabicyclo[3.2.1]octan-3-yl)-8-(5-(1-fluorocyclopropyl)-6-(trifluoromethyl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidine (Ex-79)

To a solution of tert-butyl (1R,5S)-3-(8-(5-(1-fluorocyclopropyl)-1-(tetrahydro-2H-pyran-2-yl)-6-(trifluoromethyl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate (100 mg, 0.114 mmol) in DCM (1.5 mL) was added TFA (0.5 mL, 0.114 mmol). The reaction was stirred at 30° C. for 1 h. The reaction was filtered, concentrated, and the residue was purified by reversed preparative HPLC (20-50% water/MeCN w/0.2% FA modifier) to give 4-((1R,5S)-3,8-diazabicyclo[3.2.1]octan-3-yl)-8-(5-(1-fluorocyclopropyl)-6-(trifluoromethyl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidine (Ex-79). ¹H NMR (400 MHz, CD₃OD) δ 8.82 (d, J=5.6 Hz, 1H), 8.29 (s, 1H), 7.92 (d, J=5.6 Hz, 1H), 7.69 (s, 1H), 5.40-5.46 (m, 1H), 4.53-4.65 (m, 1H), 4.48-4.50 (m, 2H), 4.41-4.46 (m, 1H), 4.12 (d, J=10.4 Hz, 2H), 3.87-4.01 (m, 2H), 3.53-3.71 (m, 3H), 3.21-3.30 (m, 1H), 2.37-2.61 (m, 2H), 2.26-2.35 (m, 1H), 2.19-2.23 (m, 2H), 1.99-2.14 (m, 5H), 0.83-1.09 (m, 3H), 0.59 (br, 1H). MS (ESI) [M+H]⁺: m/z 697.

The examples in the table below were synthesized using a similar procedure as described in the synthesis of Ex-79 by making the appropriate substitutions for starting material, intermediates, and/or reagents. Appropriate substitutions are available commercially, synthesized as described in the literature, synthesized using methods available to those skilled in the art, or synthesized as described herein.

| | | | | [M + |
|---|---|---|---|---|
| | Starting | | | H]⁺ |
| Ex. | Material | Structure | Compound Name | Found |
| Ex- 80 | 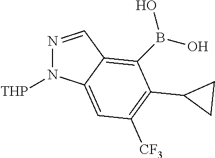 Int-46 | 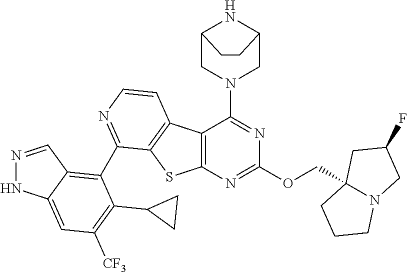 [SMILES: F[C@H]1CN2CCC[C@@]2(COc2nc(N3CC4CCC(C3)N4)c3c(n2)sc2c(-c4c(C5CC5)c(C(F)(F)F)cc5[nH]ncc45)nccc23)C1] | 4-((1R,5S)-3,8- diazabicyclo [3.2.1]octan-3- yl)-8-(5- cyclopropyl-6- (trifluoromethyl)- 1H-indazol-4-yl)- 2-(((2R,7aS)-2- fluorotetrahydro- 1H-pyrrolizin- 7a(5H)-yl) methoxy)pyrido [4′,3′:4,5]thieno [2,3-d]pyrimi- dine | 679 |

### Example 81: 4-((1R,5S)-3,8-diazabicyclo[3.2.1]octan-3-yl)-2-(2,4-dimethyl-1,4-diazepan-1-yl)-8-(6-methyl-5-(trifluoromethyl)-1H-indazol-4-yl)pyrido[4′,3′:4,5]thieno[2,3-d]pyrimidine (Ex-81)

Example 81 in the table below was synthesized using a similar procedure as described in the synthesis of Ex-25 by making the appropriate substitutions for starting material, intermediates, and/or reagents. Appropriate substitutions are available commercially, synthesized as described in the literature, synthesized using methods available to those skilled in the art, or synthesized as described herein.

| | | | | [M + | |
|---|---|---|---|---|---|
| | Starting | | | H]⁺ | Condi- |
| Ex. | materials | Structure | Compound Name | Found | tions |
| Ex- 81 | 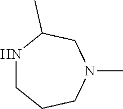 1,3-dimethyl- 1,4-diazepane | 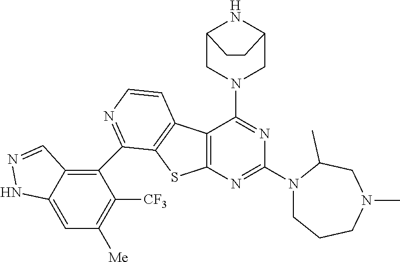 [SMILES: Cc1cc2[nH]ncc2c(-c2nccc3c2sc2nc(N4CCCN(C)CC4C)nc(N4CC5CCC(C4)N5)c23)c1C(F)(F)F] | 4-((1R,5S)-3,8-diaza- bicyclo[3.2.1]octan- 3-yl)-2-(2,4-di- methyl-1,4-diaze- pan-1-yl)-8-(6- methyl-5-(trifluoro- methyl)-1H-indazol- 4-yl)pyrido [4′,3′:4,5]thieno[2,3- d]pyrimidine | 622 | Step C: TEA (5 equiv), amine (3 equiv), THF (0.1 M), rt, 72 h. Step E not con- duct- ed. |

### Example 82: 4-((1R,5S)-3,8-diazabicyclo[3.2.1]octan-3-yl)-8-(5-ethynyl-6-fluoro-1H-thieno[3,2-f]indazol-4-yl)-2-(((2R,7aS)-2-fluorotetrahydro-TH-pyrrolizin-7a(5H)-yl)methoxy)pyrido[4′,3′:4,5]thieno[2,3-d]pyrimidine (Ex-82)

Example 82 in the table below was synthesized using a similar procedure as described in the synthesis of Ex-54 by making the appropriate substitutions for starting material, intermediates, and/or reagents. Appropriate substitutions are available commercially, synthesized as described in the literature, synthesized using methods available to those skilled in the art, or synthesized as described herein.

| | | | | [M + |
|---|---|---|---|---|
| | | | | H]⁺ |
| Ex. | Starting material | Structure | Compound Name | Found |
| Ex- 82 | 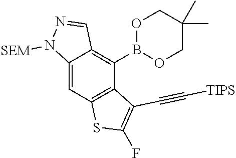 Int-23 | 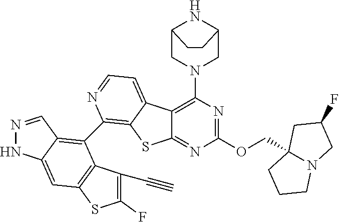 [SMILES: C#Cc1c(F)sc2cc3[nH]ncc3c(-c3nccc4c3sc3nc(OC[C@@]56CCCN5C[C@H](F)C6)nc(N5CC6CCC(C5)N6)c34)c12] | 4-((1R,5S)-3,8- diazabicyclo[3.2.1]oc- tan-3-yl)-8-(5- ethynyl-6-fluoro-1H- thieno[3,2-f]indazol- 4-yl)-2-(((2R,7aS)-2- fluorotetrahydro-1H- pyrrolizin-7a(5H)- yl)methoxy)pyri- do[4′,3′:4,5]thi- eno[2,3-d]pyrimidine | 669 |

### Example 83: 4-((1R,5S)-3,8-diazabicyclo[3.2.1]octan-3-yl)-2-(((R)-1-((6,6-difluoro-2-azaspiro[3.3]heptan-2-yl)methyl)-2,2-difluorocyclopropyl)methoxy)-8-(5-ethynyl-1H-thieno[3,2-f]indazol-4-yl)pyrido[4′,3′:4,5]thieno[2,3-d]pyrimidine (Ex-83)

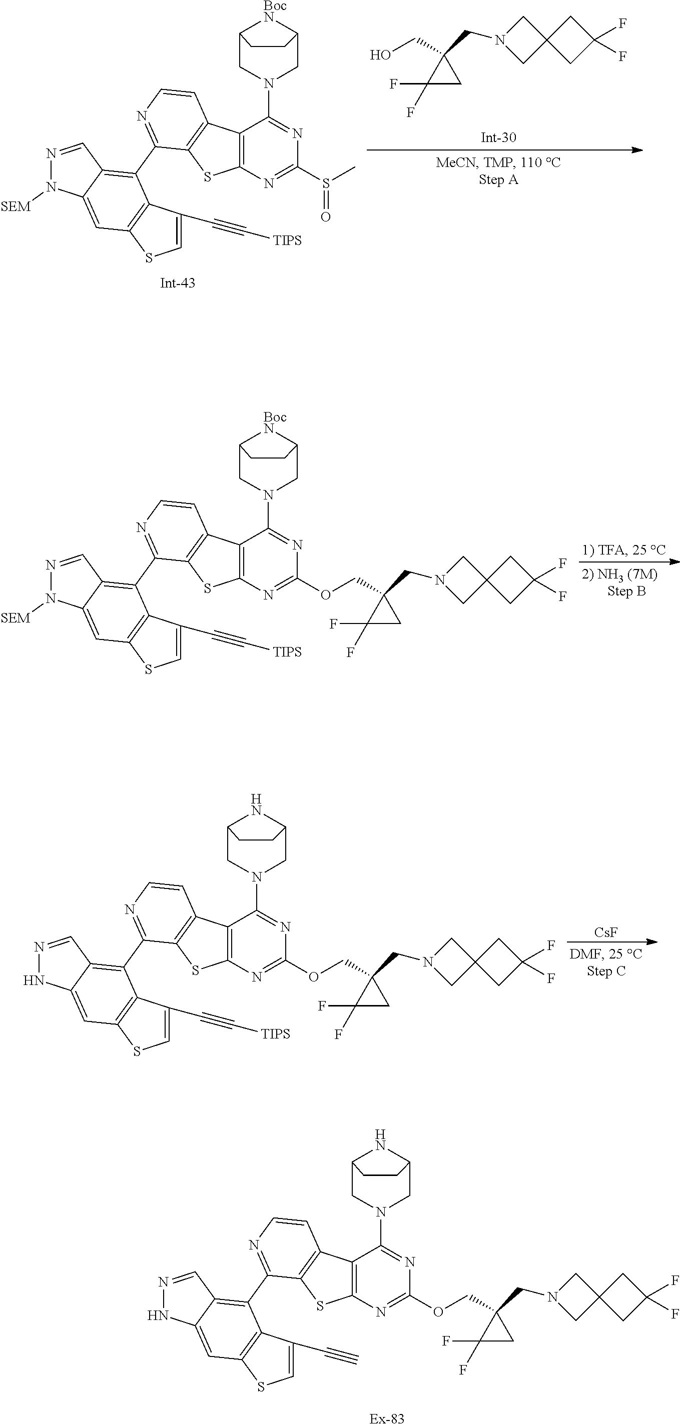

### Step A: tert-butyl (1R,5S)-3-(2-(((R)-1-((6,6-difluoro-2-azaspiro[3.3]heptan-2-yl)methyl)-2,2-difluorocyclopropyl)methoxy)-8-(5-((triisopropylsilyl)ethynyl)-1-((2-(trimethylsilyl)ethoxy)methyl)-1H-thieno[3,2-f]indazol-4-yl)pyrido[4′,3′:4,5]thieno[2,3-d]pyrimidin-4-yl)-3,8-diazabicyclo[3.2.1]octane-8-carboxylate

To a solution of tert-butyl (1R,5S)-3-(2-(methylsulfinyl)-8-(5-((triisopropylsilyl)ethynyl)-1-((2-(trimethylsilyl)ethoxy)methyl)-1H-thieno[3,2-f]indazol-4-yl)pyrido[4′,3′:4,5]thieno[2,3-d]pyrimidin-4-yl)-3,8-diazabicyclo[3.2.1]octane-8-carboxylate (Int-43) (50 mg, 0.053 mmol) and (R)-(1-((6,6-difluoro-2-azaspiro[3.3]heptan-2-yl)methyl)-2,2-difluorocyclopropyl)methanol (Int-30) (40.3 mg, 0.159 mmol) in MeCN (0.1 mL) was added 2,2,6,6-tetramethylpiperidine (150 mg, 1.06 mmol) at 25° C. The reaction was stirred at 110° C. for 16 h. The reaction was diluted with DCM (1 mL) and purified by prep-TLC (Pet. ether:THF=2:1) to give tert-butyl (1R,5S)-3-(2-(((R)-1-((6,6-difluoro-2-azaspiro[3.3]heptan-2-yl)methyl)-2,2-difluorocyclopropyl)methoxy)-8-(5-((triisopropylsilyl)ethynyl)-1-((2-(trimethylsilyl)ethoxy)methyl)-1H-thieno[3,2-f]indazol-4-yl)pyrido[4′,3′:4,5]thieno[2,3-d]pyrimidin-4-yl)-3,8-diazabicyclo[3.2.1]octane-8-carboxylate. MS (ESI) [M+H]⁺: m/z 1131.

### Step B: 4-((1R,5S)-3,8-diazabicyclo[3.2.1]octan-3-yl)-2-(((R)-1-((6,6-difluoro-2-azaspiro[3.3]heptan-2-yl)methyl)-2,2-difluorocyclopropyl)methoxy)-8-(5-((triisopropylsilyl)ethynyl)-1H-thieno[3,2-f]indazol-4-yl)pyrido[4′,3′:4,5]thieno[2,3-d]pyrimidine

A solution of tert-butyl (1R,5S)-3-(2-(((R)-1-((6,6-difluoro-2-azaspiro[3.3]heptan-2-yl)methyl)-2,2-difluorocyclopropyl)methoxy)-8-(5-((triisopropylsilyl)ethynyl)-1-((2-(trimethylsilyl)ethoxy)methyl)-1H-thieno[3,2-f]indazol-4-yl)pyrido[4′,3′:4,5]thieno[2,3-d]pyrimidin-4-yl)-3,8-diazabicyclo[3.2.1]octane-8-carboxylate (47 mg, 0.042 mmol) in TFA (1 mL, 13.0 mmol) was stirred at 25° C. for 1 h. Then the mixture was concentrated in vacuo, and the residue was diluted with NH₃ (0.5 mL, 7 M NH₃ in MeOH) and was stirred at 25° C. for 1 h. The mixture was concentrated in vacuo to give 4-((1R,5S)-3,8-diazabicyclo[3.2.1]octan-3-yl)-2-(((R)-1-((6,6-difluoro-2-azaspiro[3.3]heptan-2-yl)methyl)-2,2-difluorocyclopropyl)methoxy)-8-(5-((triisopropylsilyl)ethynyl)-1H-thieno[3,2-f]indazol-4-yl)pyrido[4′,3′:4,5]thieno[2,3-d]pyrimidine. MS (ESI) [M+H]⁺: m/z 901.

### Step C: 4-((1R,5S)-3,8-diazabicyclo[3.2.1]octan-3-yl)-2-(((R)-1-((6,6-difluoro-2-azaspiro[3.3]heptan-2-yl)methyl)-2,2-difluorocyclopropyl)methoxy)-8-(5-ethynyl-1H-thieno[3,2-]indazol-4-yl)pyrido[4′,3′:4,5]thieno[2,3-d]pyrimidine (Ex-83)

To a solution of 4-((1R,5S)-3,8-diazabicyclo[3.2.1]octan-3-yl)-2-(((R)-1-((6,6-difluoro-2-azaspiro[3.3]heptan-2-yl)methyl)-2,2-difluorocyclopropyl)methoxy)-8-(5-((triisopropylsilyl)ethynyl)-1H-thieno[3,2-J]indazol-4-yl)pyrido[4′,3′:4,5]thieno[2,3-d]pyrimidine (37.4 mg, 0.042 mmol) in DMF (0.3 mL) was added CsF (63.0 mg, 0.415 mmol) at 25° C. The reaction was stirred at 25° C. for 1 h. The reaction was filtered, and the filtrate was diluted with MeCN (1 mL), then purified by preparative HPLC (50-100% water/MeOH w/0.05% NH₄OH+10 mM NH₄HCO₃) to give 4-((1R,5S)-3,8-diazabicyclo[3.2.1]octan-3-yl)-2-(((R)-1-((6,6-difluoro-2-azaspiro[3.3]heptan-2-yl)methyl)-2,2-difluorocyclopropyl)methoxy)-8-(5-ethynyl-1H-thieno[3,2-f]indazol-4-yl)pyrido[4′,3′:4,5]thieno[2,3-d]pyrimidine (Ex-83). ¹H NMR (400 MHz, CD₃OD) δ 8.70 (d, J=5.7 Hz, 1H), 8.28 (d, J=0.8 Hz, 1H), 7.85 (d, J=5.7 Hz, 1H), 7.82 (s, 2H), 4.56 (br t, J=11.5 Hz, 1H), 4.47-4.34 (m, 2H), 4.18 (br d, J=12.9 Hz, 1H), 3.76 (br d, J=12.6 Hz, 1H), 3.62 (br d, J=11.8 Hz, 3H), 3.35 (d, J=1.1 Hz, 4H), 2.82 (d, J=1.9 Hz, 1H), 2.80-2.70 (m, 2H), 2.62 (dt, J=1.4, 12.2 Hz, 4H), 1.91-1.65 (m, 4H), 1.60-1.50 (m, 1H), 1.44 (dt, J=3.5, 8.1 Hz, 1H). MS (ESI) [M+H]⁺: m/z 745.

Ex-84 in the table below was synthesized using a similar procedure as described in the synthesis of Ex-83 by making the appropriate substitutions for starting material, intermediates, and/or reagents. Appropriate substitutions are available commercially, synthesized as described in the literature, synthesized using methods available to those skilled in the art, or synthesized as described herein.

| | | | | [M + | |
|---|---|---|---|---|---|
| | Starting | | | H]⁺ | Condi- |
| Ex. | material | Structure | Compound Name | Found | tions |
| Ex- 84 | 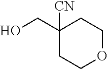 | 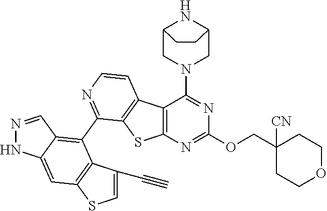 [SMILES: C#Cc1csc2cc3[nH]ncc3c(-c3nccc4c3sc3nc(OCC5(C#N)CCOCC5)nc(N5CC6CCC(C5)N6)c34)c12] | 4-(((4-((1R,5S)-3,8- diazabicyclo[3.2.1]oc- tan-3-yl)-8-(5- ethynyl-1H- thieno[3,2-f]indazol-4- yl)pyrido[4′,3′:4,5]thi- eno[2,3-d]pyrimi- din-2-yl)oxy) methyl)tetrahydro- 2H-pyran-4- carbonitrile | 633 | Step A: DBU (5 equiv), alcohol (2 equiv) MeCN, rt |

### Example 85: 4-((1R,5S)-3,8-diazabicyclo[3.2.1]octan-3-yl)-8-(5-ethynyl-1H-thieno[3,2-f]indazol-4-yl)-2-(((S,Z)-2-(fluoromethylene)tetrahydro-1H-pyrrolizin-7a(5H)-yl)methoxy)pyrido[4′,3′:4,5]thieno[2,3-d]pyrimidine (Ex-85)

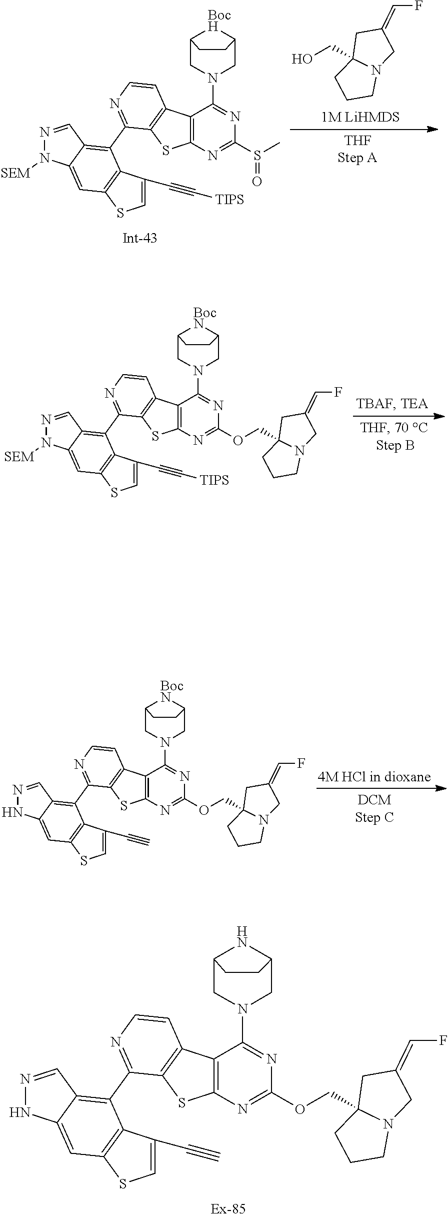

### Step A: tert-butyl (1R,5S)-3-(2-(((S,Z)-2-(fluoromethylene)tetrahydro-1H-pyrrolizin-7a(5H)-yl)methoxy)-8-(5-((triisopropylsilyl)ethynyl)-1-((2-(trimethylsilyl)ethoxy)methyl)-1H-thieno[3,2-f]indazol-4-yl)pyrido[4′,3′:4,5]thieno[2,3-d]pyrimidin-4-yl)-3,8-diazabicyclo[3.2.1]octane-8-carboxylate

To a vial was added (S,Z)-(2-(fluoromethylene)tetrahydro-1H-pyrrolizin-7a(5H)-yl)methanol (Int-27) (36.3 mg, 0.212 mmol) in THF (1 mL) under nitrogen. To this solution, LiHMDS (0.318 mL, 0.318 mmol, 1 M in THF) was added and stirred at rt for 10 mins. In a separate vial was added tert-butyl (1R,5S)-3-(2-(methylsulfinyl)-8-(5-((triisopropylsilyl)ethynyl)-1-((2-(trimethylsilyl)ethoxy)methyl)-1H-thieno[3,2-f]indazol-4-yl)pyrido[4′,3′:4,5]thieno[2,3-d]pyrimidin-4-yl)-3,8-diazabicyclo[3.2.1]octane-8-carboxylate (Int-43) (100 mg, 0.106 mmol) and THF (1 mL) and to this was added the above mixture and stirred for 30 mins. The reaction mixture was diluted with EtOAc, washed with water, brine, and the organic layer was concentrated under reduced pressure. The product was purified by silica gel column (0-100% EtOAc in hexanes) to attain tert-butyl (1R,5S)-3-(2-(((S,Z)-2-(fluoromethylene)tetrahydro-1H-pyrrolizin-7a(5H)-yl)methoxy)-8-(5-((triisopropylsilyl)ethynyl)-1-((2-(trimethylsilyl)ethoxy)methyl)-1H-thieno[3,2-f]indazol-4-yl)pyrido[4′,3′:4,5]thieno[2,3-d]pyrimidin-4-yl)-3,8-diazabicyclo[3.2.1]octane-8-carboxylate. MS (ESI) [M+H]⁺: m/z 1049.

### Step B: tert-butyl (1R,5S)-3-(8-(5-ethynyl-1H-thieno[3,2-f]indazol-4-yl)-2-(((S,Z)-2-(fluoromethylene)tetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate

To a solution of tert-butyl (1R,5S)-3-(2-(((S,Z)-2-(fluoromethylene)tetrahydro-1H-pyrrolizin-7a(5H)-yl)methoxy)-8-(5-((triisopropylsilyl)ethynyl)-1-((2-(trimethylsilyl)ethoxy)methyl)-1H-thieno[3,2-f]indazol-4-yl)pyrido[4′,3′:4,5]thieno[2,3-d]pyrimidin-4-yl)-3,8-diazabicyclo[3.2.1]octane-8-carboxylate (69 mg, 0.066 mmol) in THF (1 mL) was added TEA (4.58 μL, 0.033 mmol) followed by tetrabutylammonium fluoride (0.329 ml, 0.329 mmol). The reaction mixture was stirred at 70° C. for 4 h. Upon cooling, the reaction mixture was diluted with EtOAc, washed with water, brine, and the organic layer was concentrated under reduced pressure. The product was purified by silica gel column (0-100% of EtOAc in hexane) to get tert-butyl (1R,5S)-3-(8-(5-ethynyl-1H-thieno[3,2-f]indazol-4-yl)-2-(((S,Z)-2-(fluoromethylene)tetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate. MS (ESI) [M+H]⁺: m/z 763.

### Step C: 4-((1R,5S)-3,8-diazabicyclo[3.2.1]octan-3-yl)-8-(5-ethynyl-1H-thieno[3,2-f]indazol-4-yl)-2-(((S,Z)-2-(fluoromethylene)tetrahydro-1H-pyrrolizin-7a(5H)-yl)methoxy)pyrido[4′,3′:4,5]thieno[2,3-d]pyrimidine (Ex-85)

To a solution of tert-butyl (1R,5S)-3-(8-(5-ethynyl-1H-thieno[3,2-f]indazol-4-yl)-2-(((S,Z)-2-(fluoromethylene)tetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate (16 mg, 0.021 mmol) in DCM (0.5 mL) was added HCl (0.131 ml, 0.524 mmol). The mixture was stirred at RT for 2 h. The reaction mixture was diluted with DCM, washed with sat. aq. NaHCO₃, brine, and the organic layer was dried over Na₂SO₄, filtered, and the filtrate was concentrated under reduced pressure. The product was purified by silica gel column chromatography (0-100% of methanol in DCM), the again purified by HPLC (water/MeCN w/0.1% NH₄OH modifier) to yield 4-((1R,5S)-3,8-diazabicyclo[3.2.1]octan-3-yl)-8-(5-ethynyl-1H-thieno[3,2-J]indazol-4-yl)-2-(((S,Z)-2-(fluoromethylene)tetrahydro-1H-pyrrolizin-7a(5H)-yl)methoxy)pyrido[4′,3′:4,5]thieno[2,3-d]pyrimidine (Ex-85). ¹H NMR (600 MHz, DMSO) δ 13.39 (s, 2H), 8.78-8.68 (m, 1H), 8.40 (d, J=1.0 Hz, 1H), 8.01 (s, 1H), 7.81 (s, 1H), 7.74 (d, J=5.6 Hz, 1H), 6.81 (s, 1H), 6.67 (s, 1H), 4.18 (d, J=12.2 Hz, 1H), 4.09-3.91 (m, 4H), 3.72-3.61 (m, 2H), 3.58-3.42 (m, 4H), 3.31 (s, 1H), 3.30-3.22 (m, 2H), 2.98 (ddd, J=10.2, 6.5, 4.2 Hz, 2H), 2.58-2.53 (m, 2H), 2.30 (d, J=15.4 Hz, 1H), 1.97-1.81 (m, 3H), 1.81-1.72 (m, 1H), 1.71-1.64 (m, 2H), 1.63-1.50 (m, 3H). MS (ESI) [M+H]⁺: m/z 663.

Example 86: 4-((1R,5S)-3,8-diazabicyclo[3.2.1]octan-3-yl)-8-(5-ethynyl-7-fluoro-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidine (Ex-86)

### Step A: tert-butyl (1R,5S)-3-(8-(7-fluoro-1-(tetrahydro-2H-pyran-2-yl)-5-((triisopropylsilyl)ethynyl)-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate

To a solution of tert-butyl (1R,5S)-3-(8-chloro-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate (Int-19) (90 mg, 0.153 mmol) in toluene (1 mL) was added (7-fluoro-1-(tetrahydro-2H-pyran-2-yl)-5-((triisopropylsilyl)ethynyl)-1H-benzo[f]indazol-4-yl)boronic acid (Int-25) (91 mg, 0.183 mmol), chloro[(di(1-adamantyl)-N-butylphosphine)-2-(2-aminobiphenyl)]palladium(II) (51.1 mg, 0.076 mmol) and cesium carbonate (0.229 mL, 0.458 mmol) (2 M in water) at 25° C. under N₂ atmosphere. The mixture was stirred at 80° C. for 12 h. The reaction mixture was diluted with water (1 mL) and then extracted with EtOAc (20 mL*2). The combined organic layer was dried with Na₂SO₄, filtered, and the solvent was removed under reduced pressure. The material was purified by prep-TLC (DCM:MeOH=10:1) to give tert-butyl (1R,5S)-3-(8-(7-fluoro-1-(tetrahydro-2H-pyran-2-yl)-5-((triisopropylsilyl)ethynyl)-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate. MS (ESI) [M+H]⁺: m/z 1003.

### Step B: 4-((1R,5S)-3,8-diazabicyclo[3.2.1]octan-3-yl)-8-(7-fluoro-5-((triisopropylsilyl)ethynyl)-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidine

A solution of tert-butyl (1R,5S)-3-(8-(7-fluoro-1-(tetrahydro-2H-pyran-2-yl)-5-((triisopropylsilyl)ethynyl)-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate (90 mg, 0.090 mmol) in TFA (1 mL) was stirred at 25° C. for 30 min. The mixture was concentrated under reduced pressure. The residue was basified by aq. NaHCO₃ (0.5 mL) and extracted with DCM (10 mL*2). The combined organic layer was dried with Na₂SO₄, filtered, and the filtrate was concentrated under reduced pressure to give 4-((1R,5S)-3,8-diazabicyclo[3.2.1]octan-3-yl)-8-(7-fluoro-5-((triisopropylsilyl)ethynyl)-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidine. MS (ESI) [M+H]⁺: m/z 819.

### Step C: 4-((1R,5S)-3,8-diazabicyclo[3.2.1]octan-3-yl)-8-(5-ethynyl-7-fluoro-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidine (Ex-86)

To a solution of 4-((1R,5S)-3,8-diazabicyclo[3.2.1]octan-3-yl)-8-(7-fluoro-5-((triisopropylsilyl)ethynyl)-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidine (73.5 mg, 0.090 mmol) in DMF (1 mL) was added CsF (204 mg, 1.35 mmol) at 25° C. The mixture was stirred at 25° C. for 1 h. The mixture was filtered, and the filtrate was concentrated under reduced pressure. The material was purified by prep-HPLC (6-36% water/MeCN w/0.2% FA modifier) to give 4-((1R,5S)-3,8-diazabicyclo[3.2.1]octan-3-yl)-8-(5-ethynyl-7-fluoro-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidine (Ex-86). ¹H NMR (400 MHz, CD₃OD) δ 8.77 (d, J=5.60 Hz, 1H), 8.30 (s, 1H), 7.94 (d, J=5.72 Hz, 1H), 7.88 (dd, J=2.50, 9.78 Hz, 1H), 7.79 (s, 1H), 7.46 (dd, J=2.62, 8.58 Hz, 1H), 5.35-5.56 (m, 1H), 4.62 (br dd, J=5.84, 13.47 Hz, 1H), 4.43-4.51 (m, 2H), 4.36 (br d, J=13.59 Hz, 1H), 4.01-4.18 (m, 3H), 3.88 (br d, J=14.31 Hz, 1H), 3.52-3.67 (m, 3H), 3.26 (dt, J=5.78, 10.10 Hz, 1H), 3.17 (s, 1H), 2.36-2.57 (m, 2H), 2.23-2.33 (m, 1H), 2.13-2.22 (m, 3H), 1.99-2.09 (m, 3H), 1.89-1.98 (m, 1H). MS (ESI) [M+H]⁺: m/z 663.

### Example 87: 4-((1R,5S)-3,8-diazabicyclo[3.2.1]octan-3-yl)-8-(5-ethyl-6-fluoro-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidine (Ex-87)

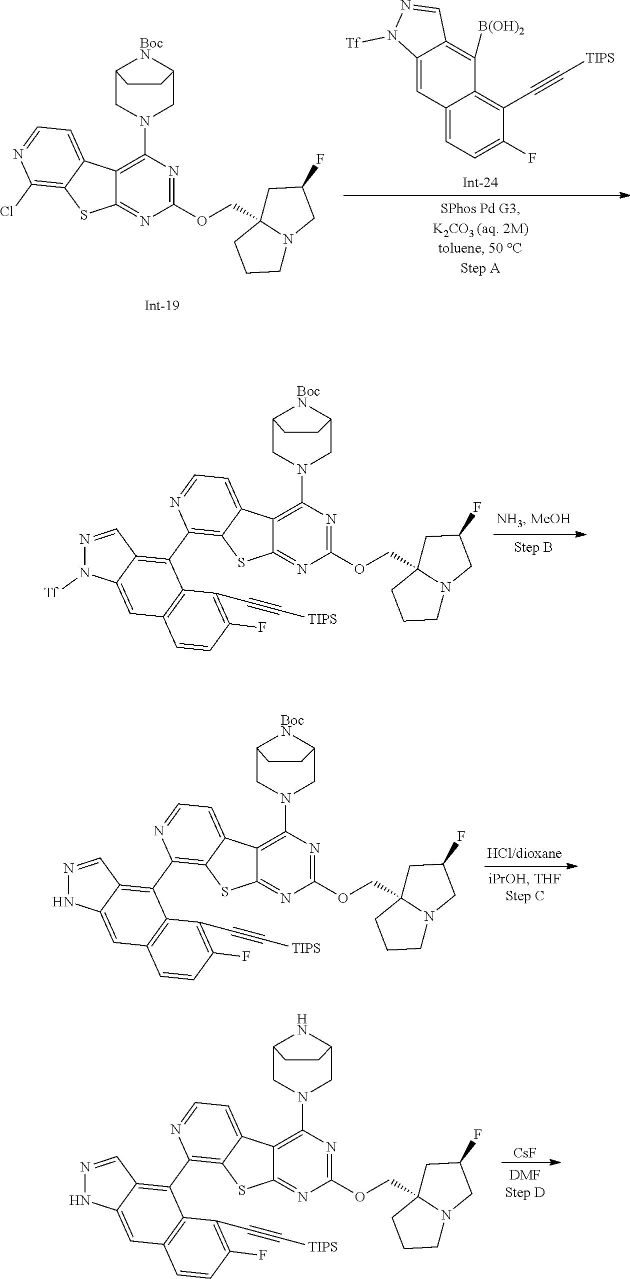

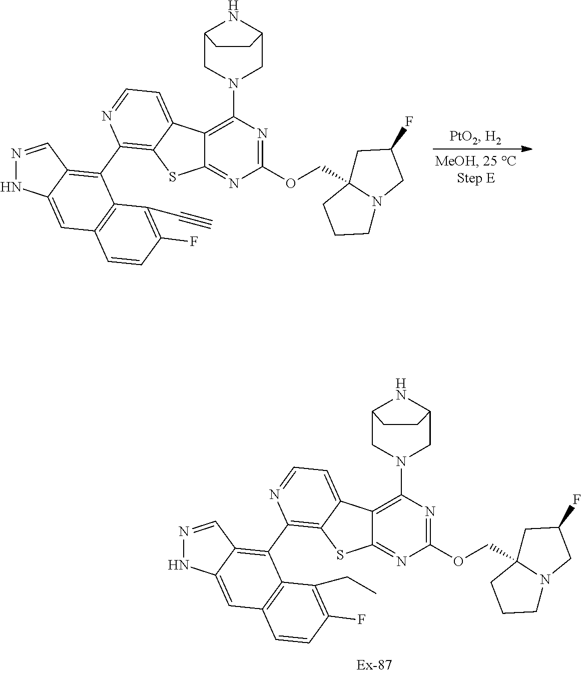

### Step A: tert-butyl (1R,5S)-3-(8-(6-fluoro-1-((trifluoromethyl)sulfonyl)-5-((triisopropylsilyl)ethynyl)-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate

To a solution of tert-butyl (1R,5S)-3-(8-chloro-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate (Int-19) (200 mg, 0.339 mmol) and (6-fluoro-1-((trifluoromethyl)sulfonyl)-5-((triisopropylsilyl)ethynyl)-1H-benzo[f]indazol-4-yl)boronic acid (Int-24) (203 mg, 0.373 mmol) in toluene (6 mL) was added SPhos Pd G3 (132 mg, 0.170 mmol) and K₂CO₃ (0.509 mL, 1.02 mmol, 2 M aqueous solution) in a glove box, and the mixture was stirred at 50° C. for 4 h under N₂ atmosphere. The reaction was diluted with EtOAc (15 mL), and the mixture was washed with brine (3 mL). The organic phase was dried over anhydrous Na₂SO₄, filtered and concentrated in vacuo. The residue was purified by preparative TLC plate (Pet. ether/THF=1/1) to give tert-butyl (1R,5S)-3-(8-(6-fluoro-1-((trifluoromethyl)sulfonyl)-5-((triisopropylsilyl)ethynyl)-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate. MS (ESI) [M+H]⁺: m/z 1051.

### Step B: tert-butyl (1R,5S)-3-(8-(6-fluoro-5-((triisopropylsilyl)ethynyl)-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate

To a solution of tert-butyl (1R,5S)-3-(8-(6-fluoro-1-((trifluoromethyl)sulfonyl)-5-((triisopropylsilyl)ethynyl)-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate (170 mg, 0.162 mmol) in DCM (0.5 mL) was added ammonia (5 mL, 35.0 mmol) (7 M in MeOH), and the mixture was stirred at 25° C. for 12 h. The reaction was concentrated in vacuo, and the residue was purified by preparative TLC plate (DCM/MeOH=10/1) to give tert-butyl (1R,5S)-3-(8-(6-fluoro-5-((triisopropylsilyl)ethynyl)-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate. MS (ESI) [M+H]⁺: m/z 919.

### Step C: 4-((1R,5S)-3,8-diazabicyclo[3.2.1]octan-3-yl)-8-(6-fluoro-5-((triisopropylsilyl)ethynyl)-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidine

To a solution of tert-butyl (1R,5S)-3-(8-(6-fluoro-5-((triisopropylsilyl)ethynyl)-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate (160 mg, 0.174 mmol) in 2-propanol (0.5 mL) and THF (0.5 mL) was added HCl (1 mL, 4.00 mmol, 4 M dioxane solution), and the mixture was stirred at 25° C. for 2 h. The reaction was concentrated in vacuo, and the residue was diluted with DCM (contained 10% MeOH) (20 mL), and basified with sat. aq. NaHCO₃. The organic phase was washed with brine, dried over anhydrous Na₂SO₄, filtered and concentrated in vacuo to give 4-((1R,5S)-3,8-diazabicyclo[3.2.1]octan-3-yl)-8-(6-fluoro-5-((triisopropylsilyl)ethynyl)-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidine. MS (ESI) [M+H]⁺: m/z 819.

### Step D: 4-((1R,5S)-3,8-diazabicyclo[3.2.1]octan-3-yl)-8-(5-ethynyl-6-fluoro-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidine

To a solution of 4-((1R,5S)-3,8-diazabicyclo[3.2.1]octan-3-yl)-8-(6-fluoro-5-((triisopropylsilyl)ethynyl)-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidine (120 mg, 0.147 mmol) in DMF (4 mL) was added CsF (223 mg, 1.47 mmol), and the mixture was stirred at 25° C. for 2 h. The reaction mixture was filtered, and the mixture was purified by preparative HPLC (42-72% water/MeCN w/0.04% NH₄OH+10 mM NH₄HCO₃ modifier) to give 4-((1R,5S)-3,8-diazabicyclo[3.2.1]octan-3-yl)-8-(5-ethynyl-6-fluoro-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidine. MS (ESI) [M+H]⁺: m/z 663.

### Step E: 4-((1R,5S)-3,8-diazabicyclo[3.2.1]octan-3-yl)-8-(5-ethyl-6-fluoro-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidine (Ex-87)

To a solution of 4-((1R,5S)-3,8-diazabicyclo[3.2.1]octan-3-yl)-8-(5-ethynyl-6-fluoro-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidine (60 mg, 0.091 mmol) in MeOH (8 mL) was added PtO₂ (100 mg, 0.440 mmol) at 25° C. under N₂ atmosphere. The mixture was degassed and purged with hydrogen three times and stirred at 25° C. for 3 h under H₂ atmophere (15 psi). The reaction mixture was filtered, and the filter cake was washed with MeOH (10 mL×2). The filtrate was concentrated in vacuo. The material was purified by prep-HPLC (10-30% water/MeCN w/0.2% FA modifier) to give 4-((1R,5S)-3,8-diazabicyclo[3.2.1]octan-3-yl)-8-(5-ethyl-6-fluoro-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidine (Ex-87). ¹H NMR (400 MHz, CD₃OD) δ 8.83 (d, J=5.60 Hz, 1H), 8.32 (s, 1H), 8.08 (dd, J=5.96, 9.30 Hz, 1H), 7.94 (d, J=5.60 Hz, 1H), 7.60 (s, 1H), 7.37 (t, J=9.36 Hz, 1H), 5.30-5.46 (m, 1H), 4.57 (br s, 1H), 4.32-4.44 (m, 3H), 3.91-4.07 (m, 3H), 3.84 (br d, J=13.23 Hz, 1H), 3.39-3.52 (m, 3H), 3.13-3.21 (m, 1H), 2.31-2.51 (m, 3H), 2.18-2.25 (m, 1H), 2.08-2.17 (m, 4H), 1.99 (br d, J=6.79 Hz, 4H), 0.76 (t, J=7.39 Hz, 3H). MS (ESI) [M+H]⁺: m/z 667.

### Example 88: 4-((1R,5S)-3-(8-(5-ethynyl-6-fluoro-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octan-8-yl)butanenitrile (Ex-88)

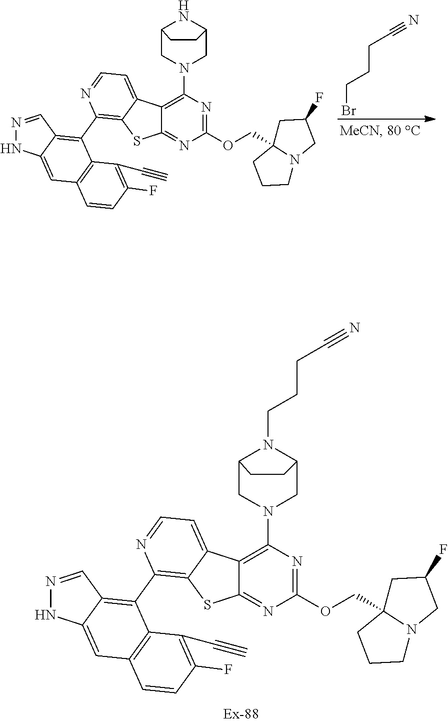

To a solution of 4-((1R,5S)-3,8-diazabicyclo[3.2.1]octan-3-yl)-8-(5-ethynyl-6-fluoro-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidine (40 mg, 0.060 mmol) in MeCN (3 mL) was added 4-bromobutanenitrile (8.93 mg, 0.060 mmol) at 25° C. under N₂. The mixture was stirred at 80° C. for 9 h. The mixture was filtered and the filtrate was purified by prep-HPLC (45-75% water/MeCN w/0.04% NH₄OH+10 mM NH₄HCO₃ modifier) to give 4-((1R,5S)-3-(8-(5-ethynyl-6-fluoro-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-3,8-diazabicyclo[3.2.1]octan-8-yl)butanenitrile (Ex-88). ¹H NMR (400 MHz, CD₃OD) δ 8.70 (d, J=5.60 Hz, 1H), 8.37 (s, 1H), 8.24 (dd, J=5.84, 9.42 Hz, 1H), 7.90 (d, J=5.72 Hz, 1H), 7.81 (d, J=0.95 Hz, 1H), 7.42 (t, J=8.94 Hz, 1H), 5.17-5.39 (m, 1H), 4.40-4.47 (m, 1H), 4.22-4.27 (m, 1H), 4.12-4.21 (m, 2H), 3.83-3.88 (m, 1H), 3.68 (br d, J=13.11 Hz, 1H), 3.36-3.44 (m, 2H), 3.31 (s, 1H), 3.19-3.26 (m, 2H), 3.16 (s, 1H), 3.00 (dt, J=5.54, 9.45 Hz, 1H), 2.65-2.70 (m, 2H), 2.57 (t, J=6.79 Hz, 2H), 2.22-2.33 (m, 1H), 2.15-2.21 (m, 1H), 2.05-2.13 (m, 1H), 1.96-2.03 (m, 2H), 1.84-1.95 (m, 5H), 1.74-1.82 (m, 1H), 1.54-1.66 (m, 1H). MS (ESI) [M+H]⁺: m/z 730.

### Example 89: 4-((1R,5S)-3,8-diazabicyclo[3.2.1]octan-3-yl)-8-(5-ethynyl-6-fluoro-1H-benzo[f]indazol-4-yl)-2-(((S,Z)-2-(fluoromethylene)tetrahydro-1H-pyrrolizin-7a(5H)-yl)methoxy)pyrido[4′,3′:4,5]thieno[2,3-d]pyrimidine (Ex-89)

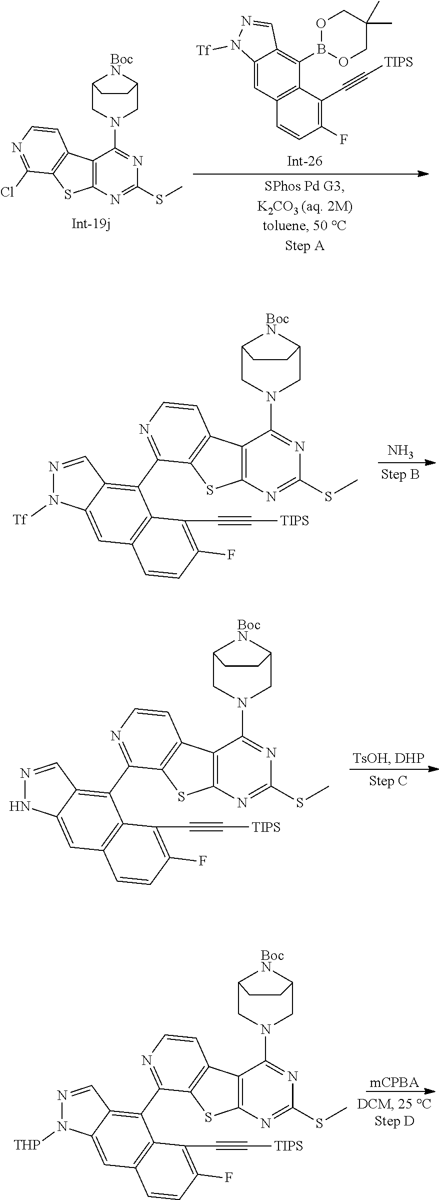

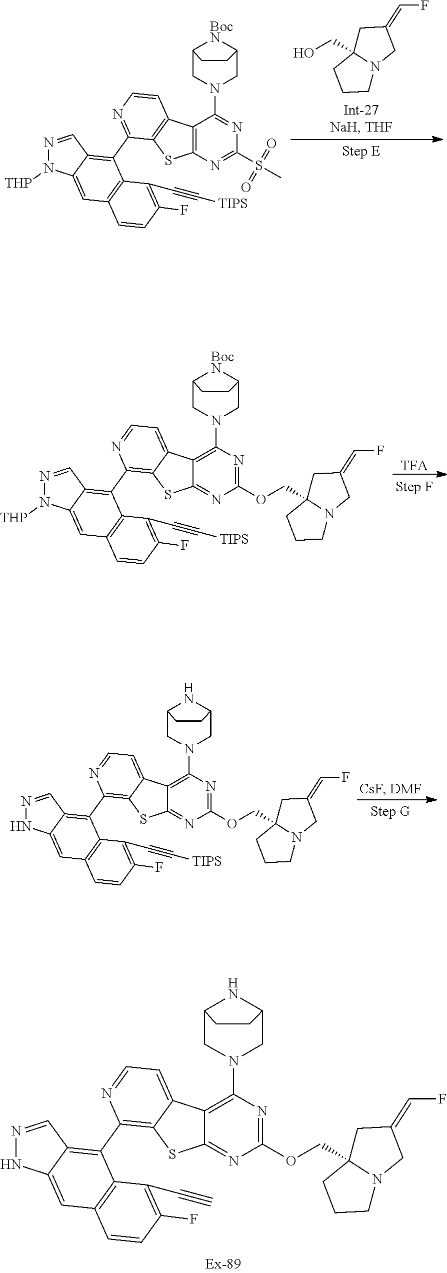

### Step A: tert-butyl (1R,5S)-3-(8-(6-fluoro-1-((trifluoromethyl)sulfonyl)-5-((triisopropylsilyl)ethynyl)-1H-benzo[f]isoindol-4-yl)-2-(methylthio)pyrido[4′,3′:4,5]thieno[2,3-d]pyrimidin-4-yl)-3,8-diazabicyclo[3.2.1]octane-8-carboxylate

To a mixture of tert-butyl (1R,5S)-3-(8-chloro-2-(methylthio)pyrido[4′,3′:4,5]thieno[2,3-d]pyrimidin-4-yl)-3,8-diazabicyclo[3.2.1]octane-8-carboxylate (Int-19J) (4.00 g, 8.37 mmol), 4-(5,5-dimethyl-1,3,2-dioxaborinan-2-yl)-6-fluoro-1-((trifluoromethyl)sulfonyl)-5-((triisopropylsilyl)ethynyl)-1H-benzo[f]indazole (Int-26) (5.62 g, 9.20 mmol) and SPhos Pd G3 (3.26 g, 4.18 mmol) in toluene (40 mL) was added K₂CO₃ (12.6 mL, 25.1 mmol, 2 M in H₂O) at 25° C. The mixture was stirred at 50° C. for 1 h under N₂ atmosphere. The reaction mixture was extracted with EtOAc (30 mL×3). The organic layer was dried over sodium sulfate, filtered and concentrated under reduced pressure. The residue was purified by flash silica gel chromatography (20% ethyl acetate in petroleum ether) to give tert-butyl (1R,5S)-3-(8-(6-fluoro-1-((trifluoromethyl)sulfonyl)-5-((triisopropylsilyl)ethynyl)-1H-benzo[f]isoindol-4-yl)-2-(methylthio)pyrido[4′,3′:4,5]thieno[2,3-d]pyrimidin-4-yl)-3,8-diazabicyclo[3.2.1]octane-8-carboxylate. MS (ESI) [M+H]⁺: m/z 940.

### Step B: tert-butyl (1R,5S)-3-(8-(6-fluoro-5-((triisopropylsilyl)ethynyl)-1H-benzo[f]indazol-4-yl)-2-(methylthio)pyrido[4′,3′:4,5]thieno[2,3-d]pyrimidin-4-yl)-3,8-diazabicyclo[3.2.1]octane-8-carboxylate

To a solution of tert-butyl (1R,5S)-3-(8-(6-fluoro-1-((trifluoromethyl)sulfonyl)-5-((triisopropylsilyl)ethynyl)-1H-benzo[f]isoindol-4-yl)-2-(methylthio)pyrido[4′,3′:4,5]thieno[2,3-d]pyrimidin-4-yl)-3,8-diazabicyclo[3.2.1]octane-8-carboxylate (2.89 g, 3.07 mmol) in DCM (10 mL) was added ammonia (25 mL, 175 mmol, 7M in MeOH) at 25° C., and the mixture was stirred at 25° C. for 16 h. The reaction mixture was evaporated under reduced pressure. Then the mixture was washed with Pet. ether (30 mL), filtered, and the filter cake was collected and dried in vacuo to afford tert-butyl (1R,5S)-3-(8-(6-fluoro-5-((triisopropylsilyl)ethynyl)-1H-benzo[f]indazol-4-yl)-2-(methylthio)pyrido[4′,3′:4,5]thieno[2,3-d]pyrimidin-4-yl)-3,8-diazabicyclo[3.2.1]octane-8-carboxylate. MS (ESI) [M+H]⁺: m/z 808.

### Step C: tert-butyl (1R,5S)-3-(8-(6-fluoro-1-(tetrahydro-2H-pyran-2-yl)-5-((triisopropylsilyl)ethynyl)-1H-benzo[f]indazol-4-yl)-2-(methylthio)pyrido[4′,3′:4,5]thieno[2,3-d]pyrimidin-4-yl)-3,8-diazabicyclo[3.2.1]octane-8-carboxylate

To a solution of tert-butyl (1R,5S)-3-(8-(6-fluoro-5-((triisopropylsilyl)ethynyl)-1H-benzo[f]indazol-4-yl)-2-(methylthio)pyrido[4′,3′:4,5]thieno[2,3-d]pyrimidin-4-yl)-3,8-diazabicyclo[3.2.1]octane-8-carboxylate (410 mg, 0.507 mmol) in DCM (5 mL) was added 3,4-dihydropyran (0.093 mL, 1.02 mmol) and 4-methylbenzenesulfonic acid (8.74 mg, 0.051 mmol) at 25° C., and the mixture was stirred at 25° C. for 16 h. The mixture was concentrated in vacuo (at 25° C.), and the residue was purified by flash silica gel chromatography (40% ethyl acetate in petroleum ether) to give tert-butyl (1R,5S)-3-(8-(6-fluoro-1-(tetrahydro-2H-pyran-2-yl)-5-((triisopropylsilyl)ethynyl)-1H-benzo[f]indazol-4-yl)-2-(methylthio)pyrido[4′,3′:4,5]thieno[2,3-d]pyrimidin-4-yl)-3,8-diazabicyclo[3.2.1]octane-8-carboxylate. MS (ESI) [M+H]⁺: m/z 892.

### Step D: tert-butyl (1R,5S)-3-(8-(6-fluoro-1-(tetrahydro-2H-pyran-2-yl)-5-((triisopropylsilyl)ethynyl)-1H-benzo[f]indazol-4-yl)-2-(methylsulfonyl)pyrido[4′,3′:4,5]thieno[2,3-d]pyrimidin-4-yl)-3,8-diazabicyclo[3.2.1]octane-8-carboxylate

To a solution of tert-butyl (1R,5S)-3-(8-(6-fluoro-1-(tetrahydro-2H-pyran-2-yl)-5-((triisopropylsilyl)ethynyl)-1H-benzo[f]indazol-4-yl)-2-(methylthio)pyrido[4′,3′:4,5]thieno[2,3-d]pyrimidin-4-yl)-3,8-diazabicyclo[3.2.1]octane-8-carboxylate (430 mg, 0.482 mmol) in DCM (5 mL) was added m-CPBA (186 mg, 0.916 mmol, 85% wt) at 0° C. The mixture was removed from the ice bath and stirred at 25° C. for 30 min. The mixture was concentrated under reduced pressure. The residue was purified by preparative TLC plate (petroleum ether:ethyl acetate=1:1) to give tert-butyl (1R,5S)-3-(8-(6-fluoro-1-(tetrahydro-2H-pyran-2-yl)-5-((triisopropylsilyl)ethynyl)-1H-benzo[f]indazol-4-yl)-2-(methylsulfonyl)pyrido[4′,3′:4,5]thieno[2,3-d]pyrimidin-4-yl)-3,8-diazabicyclo[3.2.1]octane-8-carboxylate. MS (ESI) [M+H]⁺: m/z 924.

### Step E: tert-butyl (1R,5S)-3-(8-(6-fluoro-1-(tetrahydro-2H-pyran-2-yl)-5-((triisopropylsilyl)ethynyl)-1H-benzo[f]indazol-4-yl)-2-(((S,Z)-2-(fluoromethylene)tetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate

To a solution of tert-butyl (1R,5S)-3-(8-(6-fluoro-1-(tetrahydro-2H-pyran-2-yl)-5-((triisopropylsilyl)ethynyl)-1H-benzo[f]indazol-4-yl)-2-(methylsulfonyl)pyrido[4′,3′:4,5]thieno[2,3-d]pyrimidin-4-yl)-3,8-diazabicyclo[3.2.1]octane-8-carboxylate (250 mg, 0.270 mmol) and (S,Z)-(2-(fluoromethylene)tetrahydro-1H-pyrrolizin-7a(5H)-yl)methanol (Int-27) (55.6 mg, 0.325 mmol) in THF (3 mL) was added NaH (54.1 mg, 1.35 mmol, 60% wt in mineral oil) at 25° C. under N₂ atmosphere. The mixture was stirred at 25° C. for 1 h. The reaction was quenched with saturated aqueous NaHCO₃ (10 mL) and extracted with DCM (15 mL×3). The organic layer was dried over Na₂SO₄, filtered and concentrated in vacuo. The residue was purified by preparative TLC (THF:petroleum ether=1:1) to give tert-butyl (1R,5S)-3-(8-(6-fluoro-1-(tetrahydro-2H-pyran-2-yl)-5-((triisopropylsilyl)ethynyl)-1H-benzo[f]indazol-4-yl)-2-(((S,Z)-2-(fluoromethylene)tetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate. MS (ESI) [M/2+H]⁺: m/z 509.

### Step F: 4-((1R,5S)-3,8-diazabicyclo[3.2.1]octan-3-yl)-8-(6-fluoro-5-((triisopropylsilyl)ethynyl)-1H-benzo[f]indazol-4-yl)-2-(((S,Z)-2-(fluoromethylene)tetrahydro-1H-pyrrolizin-7a(5H)-yl)methoxy)pyrido[4′,3′:4,5]thieno[2,3-d]pyrimidine

A solution of tert-butyl (1R,5S)-3-(8-(6-fluoro-1-(tetrahydro-2H-pyran-2-yl)-5-((triisopropylsilyl)ethynyl)-1H-benzo[f]indazol-4-yl)-2-(((S,Z)-2-(fluoromethylene)tetrahydro-1H-pyrrolizin-7a(5H)-yl)methoxy)pyrido[4′,3′:4,5]thieno[2,3-d]pyrimidin-4-yl)-3,8-diazabicyclo[3.2.1]octane-8-carboxylate (105 mg, 0.103 mmol) in TFA (3 mL) was stirred at 20° C. for 4 h. Most of the solvent was removed by nitrogen flow, and the residue was basified with saturated aqueous NaHCO₃ (5 mL, adjust pH>7), and then extracted with EtOAc (3×15 mL). The organic layer was dried over sodium sulfate, filtered, and the solvent was evaporated under reduced pressure. The residue was purified by preparative TLC (DCM:MeOH=8:1) to give 4-((1R,5S)-3,8-diazabicyclo[3.2.1]octan-3-yl)-8-(6-fluoro-5-((triisopropylsilyl)ethynyl)-1H-benzo[f]indazol-4-yl)-2-(((S,Z)-2-(fluoromethylene)tetrahydro-1H-pyrrolizin-7a(5H)-yl)methoxy)pyrido[4′,3′:4,5]thieno[2,3-d]pyrimidine. MS (ESI) [M+H]⁺: m/z 831.

### Step G: 4-((1R,5S)-3,8-diazabicyclo[3.2.1]octan-3-yl)-8-(5-ethynyl-6-fluoro-1H-benzo[f]indazol-4-yl)-2-(((S,Z)-2-(fluoromethylene)tetrahydro-1H-pyrrolizin-7a(5H)-yl)methoxy)pyrido[4′,3′:4,5]thieno[2,3-d]pyrimidine (Ex-89)

To a solution of 4-((1R,5S)-3,8-diazabicyclo[3.2.1]octan-3-yl)-8-(6-fluoro-5-((triisopropylsilyl)ethynyl)-1H-benzo[f]indazol-4-yl)-2-(((S,Z)-2-(fluoromethylene)tetrahydro-1H-pyrrolizin-7a(5H)-yl)methoxy)pyrido[4′,3′:4,5]thieno[2,3-d]pyrimidine (80 mg, 0.096 mmol) in DMF (1 mL) was added CsF (292 mg, 1.93 mmol) at 25° C. under N₂ atmosphere. The mixture was stirred at 25° C. for 1 h. The mixture was filtered, and the filtrate was purified by reverse preparative HPLC (63-93% water/MeOH w/0.05% NH₄OH+10 mM NH₄HCO₃ modifier) to give 4-((1R,5S)-3,8-diazabicyclo[3.2.1]octan-3-yl)-8-(5-ethynyl-6-fluoro-1H-benzo[f]indazol-4-yl)-2-(((S,Z)-2-(fluoromethylene)tetrahydro-1H-pyrrolizin-7a(5H)-yl)methoxy)pyrido[4′,3′:4,5]thieno[2,3-d]pyrimidine (Ex-89). ¹H NMR (400 MHz, CD₃OD) δ 8.72 (d, J=5.7 Hz, 1H), 8.37 (s, 1H), 8.25 (dd, J=9.4, 5.8 Hz, 1H), 7.90 (d, J=5.7 Hz, 1H), 7.82 (d, J=0.8 Hz, 1H), 7.43 (t, J=8.9 Hz, 1H), 6.86-6.44 (m, 1H), 4.65 (s, 1H), 4.46 (d, J=12.2 Hz, 1H), 4.31-4.14 (m, 3H), 3.93-3.77 (m, 2H), 3.64 (d, J=13.1 Hz, 3H), 3.46 (d, J=14.3 Hz, 1H), 3.20-3.10 (m, 1H), 2.77-2.62 (m, 2H), 2.43 (d, J=15.6 Hz, 1H), 2.16-2.03 (m, 1H), 2.00-1.78 (m, 6H), 1.76-1.65 (m, 1H). MS (ESI) [M+H]⁺: m/z 675.

The examples in the table below were synthesized using a similar procedure as described in the synthesis of Ex-89 by making the appropriate substitutions for starting material, intermediates, and/or reagents. Appropriate substitutions are available commercially, synthesized as described in the literature, synthesized using methods available to those skilled in the art, or synthesized as described herein.

| | | | | [M + | |
|---|---|---|---|---|---|
| | Starting | | | H]⁺ | |
| Ex. | material | Structure | Compound Name | Found | Conditions |
| Ex- 90 | 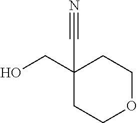 (hydroxymeth- yl)tetrahydro- 2H-pyran-4- carbonitrile | 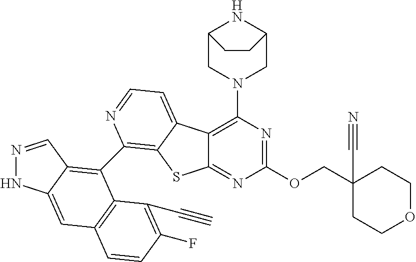 [SMILES: C#Cc1c(F)ccc2cc3[nH]ncc3c(-c3nccc4c3sc3nc(OCC5(C#N)CCOCC5)nc(N5CC6CCC(C5)N6)c34)c12] | 4-(((4-((1R,5S)-3,8- diazabicyclo[3.2.1]octan- 3-yl)-8-(5-ethynyl- 6-fluoro-1H- benzo[f]indazol-4- yl)pyrido[4′,3′:4,5] thie- no[2,3-d]pyrimidin-2- yl)oxy)methyl)tetrahy- dro-2H-pyran-4- carbonitrile | 645 | Step E: NaH (5 equiv), alcohol (1.1 equiv), THF (0.1 M), rt, 1 h |
| Ex- 91 | 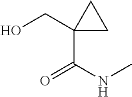 (hydroxymeth- yl)-N- methylcyclopro- pane-1- carboxamide | 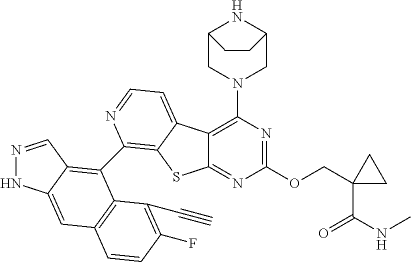 [SMILES: C#Cc1c(F)ccc2cc3[nH]ncc3c(-c3nccc4c3sc3nc(OCC5(C(=O)NC)CC5)nc(N5CC6CCC(C5)N6)c34)c12] | 1-(((4-((1R,5S)-3,8- diazabicyclo[3.2.1]octan- 3-yl)-8-(5-ethynyl- 6-fluoro-1H- benzo[f]indazol-4- yl)pyrido[4′,3′:4,5]thie- no[2,3-d]pyrimidin-2- yl)oxy)methyl)-N- methylcyclopropane- 1-carboxamide | 633 | Step E: NaH (5 equiv), alcohol (1.5 equiv), THF (0.05 M), rt, 3 h |
| Ex- 92 | 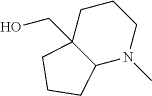 (1- methyloctahydro- 4aH- cyclopenta[b]py- ridin-4a- yl)methanol | 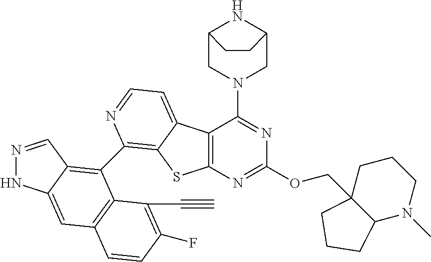 [SMILES: C#Cc1c(F)ccc2cc3[nH]ncc3c(-c3nccc4c3sc3nc(OCC56CCCC5N(C)CCC6)nc(N5CC6CCC(C5)N6)c34)c12] | 4-((1R,5S)-3,8- diazabicyclo[3.2.1]octan- 3-yl)-8-(5-ethynyl- 6-fluoro-1H- benzo[f]indazol-4-yl)- 2-((1- methyloctahydro-4aH- cyclopenta[b]pyridin- 4a- yl)methoxy)pyrido[4′, 3′:4,5]thieno[2,3- d]pyrimidine | 673 | Step E: TMP (10 equiv), alcohol (2 equiv) MeCN (0.07 M), 100 °C, 24 h |
| Ex- 93 | 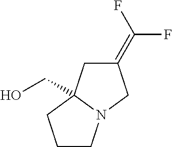 (S)-(2- (difluoromethy- lene)tetrahydro- 1H- pyrrolizin- 7a(5H)- yl)methanol | 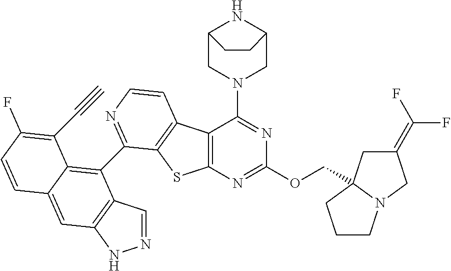 | 4-((1R,5S)-3,8- diazabicyclo[3.2.1]octan- 3-yl)-2-(((S)-2- (difluoromethylene)tetra- hydro-1H- pyrrolizin-7a(5H)- yl)methoxy)-8-(5- ethynyl-6-fluoro-1H- benzo[f]indazol-4- yl)pyrido[4′,3′:4,5] thie- no[2,3-d]pyrimidine | 693 | Step E: LiHMDS (3.6 equiv), alcohol (3 equiv), THF (0.05 M), rt, 1 h |
| Ex- 94 | 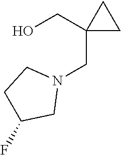 [SMILES: OCC1(CN2CC[C@@H](F)C2)CC1] (R)-(1-((3- fluoropyrrolidin- 1- yl)methyl)cyclo- propyl)metha- nol | 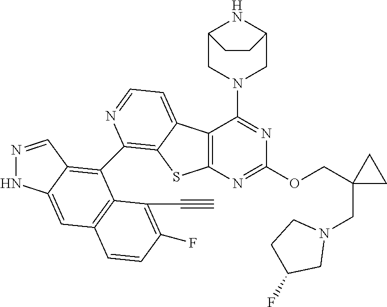 [SMILES: C#Cc1c(F)ccc2cc3[nH]ncc3c(-c3nccc4c3sc3nc(OCC5(CN6CC[C@@H](F)C6)CC5)nc(N5CC6CCC(C5)N6)c34)c12] | 4-((1R,5S)-3,8- diazabicyclo[3.2.1]octan- 3-yl)-8-(5-ethynyl- 6-fluoro-1H- benzo[f]indazol-4-yl)- 2-((1-(((R)-3- fluoropyrrolidin-1- yl)methyl)cyclopropyl) methoxy)pyrido[4′,3′: 4,5]thieno[2,3- d]pyrimidine | 677 | Step E: TMP (10 equiv), alcohol (2 equiv) MeCN (0.15 M), 100 °C, 12 h |
| Ex- 95 | 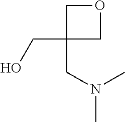 (3- ((dimethylami- no)methyl)oxe- tan-3- yl)methanol | 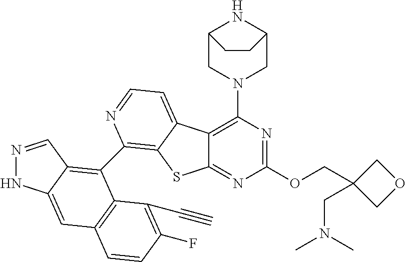 [SMILES: C#Cc1c(F)ccc2cc3[nH]ncc3c(-c3nccc4c3sc3nc(OCC5(CN(C)C)COC5)nc(N5CC6CCC(C5)N6)c34)c12] | 1-(3-(((4-((1R,5S)-3,8- diazabicyclo[3.2.1]octan- 3-yl)-8-(5-ethynyl- 6-fluoro-1H- benzo[f]indazol-4- yl)pyrido[4′,3′:4,5]thie- no[2,3-d]pyrimidin-2- yl)oxy)methyl)oxetan- 3-yl)-N,N- dimethylmethanamine | 649 | Step E: TMP (10 equiv), alcohol (2 equiv) MeCN (0.07 M), 100 ºC, 16 h |
| Ex- 96 | 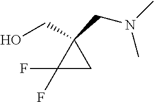 (R)-(1- ((dimethylami- no)methyl)- 2,2- difluorocyclopro- pyl)methanol | 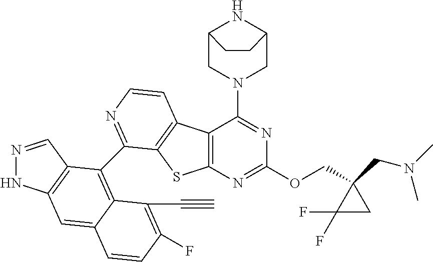 [SMILES: C#Cc1c(F)ccc2cc3[nH]ncc3c(-c3nccc4c3sc3nc(OC[C@]5(CN(C)C)CC5(F)F)nc(N5CC6CCC(C5)N6)c34)c12] | 1-((1R)-1-(((4- ((1R,5S)-3,8- diazabicyclo[3.2.1]octan- 3-yl)-8-(5-ethynyl- 6-fluoro-1H- benzo[f]indazol-4- yl)pyrido[4′,3′:4,5]thie- no[2,3-d]pyrimidin-2- yl)oxy)methyl)-2,2- difluorocyclopropyl)- N,N- dimethylmethanamine | 669 | Step E: NaH (5 equiv), alcohol (1.1 equiv), THF (0.17 M), rt, 2 h |
| Ex- 97 | 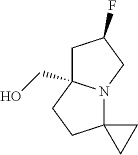 [SMILES: OC[C@@]12CCC3(CC3)N1C[C@H](F)C2] Int-28 | 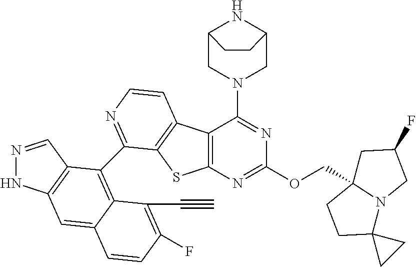 [SMILES: C#Cc1c(F)ccc2cc3[nH]ncc3c(-c3nccc4c3sc3nc(OC[C@@]56CCC7(CC7)N5C[C@H](F)C6)nc(N5CC6CCC(C5)N6)c34)c12] | 4-((1R,5S)-3,8- diazabicyclo[3.2.1]octan- 3-yl)-8-(5-ethynyl- 6-fluoro-1H- benzo[f]indazol-4-yl)- 2-(((6′R,7a′S)-6′- fluorotetrahydrospiro[cy- clopropane-1,3′- pyrrolizin]-7a′(5'H)- yl)methoxy)pyrido[4′, 3′:4,5]thieno[2,3- d]pyrimidine | 689 | Step E: LiHMDS (8 equiv), alcohol (2.5 equiv), THF (0.05 M), rt, 1 h |
| Ex- 98 | 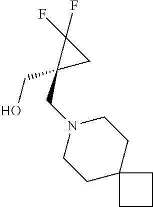 [SMILES: OC[C@@]1(CN2CCC3(CCC3)CC2)CC1(F)F] Int-31 | 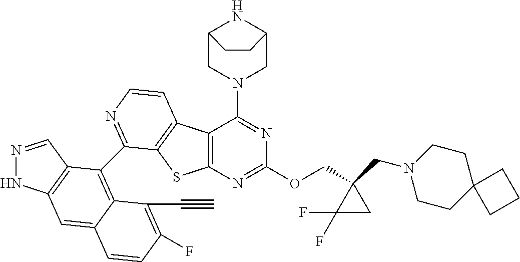 [SMILES: C#Cc1c(F)ccc2cc3[nH]ncc3c(-c3nccc4c3sc3nc(OC[C@]5(CN6CCC7(CCC7)CC6)CC5(F)F)nc(N5CC6CCC(C5)N6)c34)c12] | 2-(((R)-1-((7- azaspiro[3.5]nonan-7- yl)methyl)-2,2- difluorocyclopropyl)meth- oxy)-4-((1R,5S)- 3,8- diazabicyclo[3.2.1]octan- 3-yl)-8-(5-ethynyl- 6-fluoro-1H- benzo[f]indazol-4- yl)pyrido[4′,3′:4,5]thie- no[2,3-d]pyrimidine | 749 | Step E: NaH (5 equiv), alcohol (1 equiv), THF (0.03 M), rt, 1 h |
| Ex- 99 | 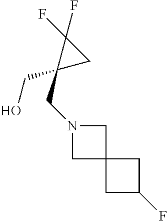 [SMILES: OC[C@@]1(CN2CC3(CC(F)C3)C2)CC1(F)F] Int-29 | 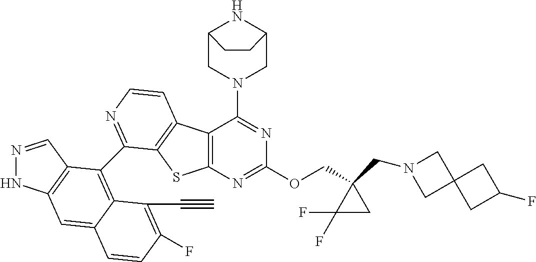 [SMILES: C#Cc1c(F)ccc2cc3[nH]ncc3c(-c3nccc4c3sc3nc(OC[C@]5(CN6CC7(CC(F)C7)C6)CC5(F)F)nc(N5CC6CCC(C5)N6)c34)c12] | 4-((1R,5S)-3,8- diazabicyclo[3.2.1]octan- 3-yl)-2-(((R)-2,2- difluoro-1-((6-fluoro- 2-azaspiro[3.3]heptan- 2- yl)methyl)cyclopropyl) methoxy)-8-(5- ethynyl-6-fluoro-1H- benzo[f]indazol-4- yl)pyrido[4′,3′:4,5]thie- no[2,3-d]pyrimidine | 739 | Step E: NaH (5 equiv), alcohol (1 equiv), THF (0.06 M), rt, 30 min |
| Ex- 100 | 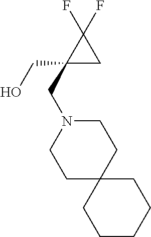 [SMILES: OC[C@@]1(CN2CCC3(CCCCC3)CC2)CC1(F)F] Int-33 | 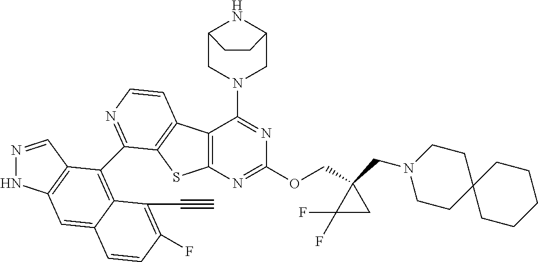 [SMILES: C#Cc1c(F)ccc2cc3[nH]ncc3c(-c3nccc4c3sc3nc(OC[C@]5(CN6CCC7(CCCCC7)CC6)CC5(F)F)nc(N5CC6CCC(C5)N6)c34)c12] | 2-(((R)-1-((3- azaspiro[5.5]undecan- 3-yl)methyl)-2,2- difluorocyclopropyl)meth- oxy)-4-((1R,5S)- 3,8- diazabicyclo[3.2.1]octan- 3-yl)-8-(5-ethynyl- 6-fluoro-1H- benzo[f]indazol-4- yl)pyrido [4′,3′:4,5]thie- no[2,3-d]pyrimidine | 777 | Step E: NaH (5 equiv), alcohol (1.1 equiv), THF (0.1 M), rt, 30 min |
| Ex- 101 | 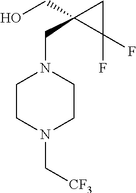 [SMILES: OC[C@]1(CN2CCN(CC(F)(F)F)CC2)CC1(F)F] Int-34 | 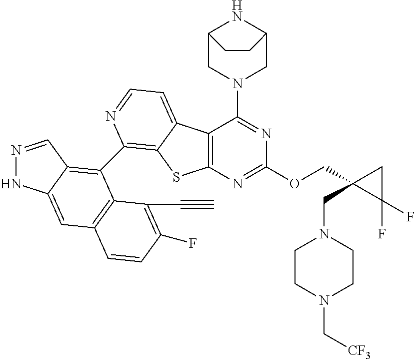 [SMILES: C#Cc1c(F)ccc2cc3[nH]ncc3c(-c3nccc4c3sc3nc(OC[C@]5(CN6CCN(CC(F)(F)F)CC6)CC5(F)F)nc(N5CC6CCC(C5)N6)c34)c12] | 4-((1R,5S)-3,8- diazabicyclo[3.2.1]octan- 3-yl)-2-(((R)-2,2- difluoro-1-((4-(2,2,2- trifluoroethyl)piperazin- 1- yl)methyl)cyclopropyl) methoxy)-8-(5- ethynyl-6-fluoro-1H- benzo[f]indazol-4- yl)pyrido[4′,3′:4,5]thie- no[2,3-d]pyrimidine | 792 | Step E: LiHMDS (3 equiv), alcohol (2 equiv), THF (0.04 M), rt, 30 min |
| Ex- 102 | 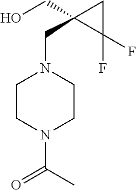 [SMILES: CC(=O)N1CCN(C[C@@]2(CO)CC2(F)F)CC1] Int-35 | 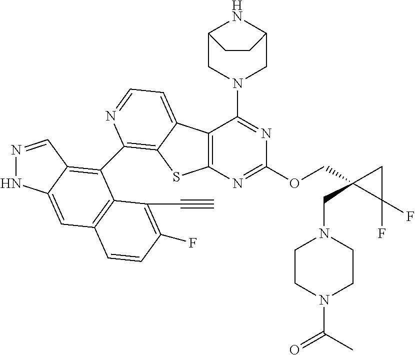 [SMILES: C#Cc1c(F)ccc2cc3[nH]ncc3c(-c3nccc4c3sc3nc(OC[C@]5(CN6CCN(C(C)=O)CC6)CC5(F)F)nc(N5CC6CCC(C5)N6)c34)c12] | 1-(4-(((1R)-1-(((4- ((1R,5S)-3,8- diazabicyclo[3.2.1]octan- 3-yl)-8-(5-ethynyl- 6-fluoro-1H- benzo[f]indazol-4- yl)pyrido [4′,3′:4,5]thie- no[2,3-d]pyrimidin-2- yl)oxy)methyl)-2,2- difluorocyclopropyl)meth- yl)piperazin-1- yl)ethan-1-one | 752 | Step E: NaH (5 equiv), alcohol (1.1 equiv), THF (0.1 M), rt, 10 min |
| Ex- 103 | 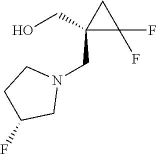 [SMILES: OC[C@]1(CN2CC[C@@H](F)C2)CC1(F)F] Int-36 | 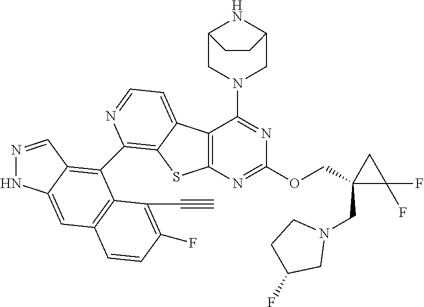 [SMILES: C#Cc1c(F)ccc2cc3[nH]ncc3c(-c3nccc4c3sc3nc(OC[C@]5(CN6CC[C@@H](F)C6)CC5(F)F)nc(N5CC6CCC(C5)N6)c34)c12] | 4-((1R,5S)-3,8- diazabicyclo[3.2.1]octan- 3-yl)-2-(((R)-2,2- difluoro-1-(((R)-3- fluoropyrrolidin-1- yl)methyl)cyclopropyl) methoxy)-8-(5- ethynyl-6-fluoro-1H- benzo[f]indazol-4- yl)pyrido[4′,3′:4,5]thie- no[2,3-d]pyrimidine | 713 | Step E: LiHMDS (3 equiv), alcohol (2 equiv), THF (0.08 M), rt, 30 min |
| Ex- 104 | 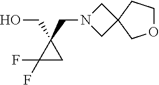 [SMILES: OC[C@]1(CN2CC3(CCOC3)C2)CC1(F)F] Int-37 | 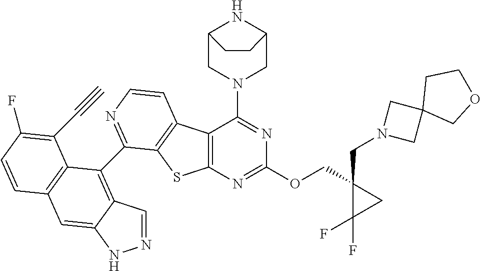 [SMILES: C#Cc1c(F)ccc2cc3[nH]ncc3c(-c3nccc4c3sc3nc(OC[C@]5(CN6CC7(CCOC7)C6)CC5(F)F)nc(N5CC6CCC(C5)N6)c34)c12] | 2-(((1R)-1-(((4- ((1R,5S)-3,8- diazabicyclo[3.2.1]octan- 3-yl)-8-(5-ethynyl- 6-fluoro-1H- benzo[f]indazol-4- yl)pyrido[4′,3′:4,5]thie- no[2,3-d]pyrimidin-2- yl)oxy)methyl)-2,2- difluorocyclopropyl)meth- yl)-6-oxa-2- azaspiro[3.4]octane | 737 | Step E: NaH (3 equiv), alcohol (1.5 equiv), THF (0.1 M), rt, 1 h |
| Ex- 105 | 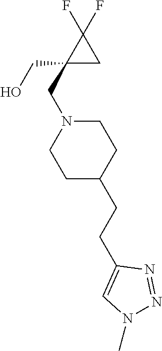 [SMILES: Cn1cc(CCC2CCN(C[C@]3(CO)CC3(F)F)CC2)nn1] | 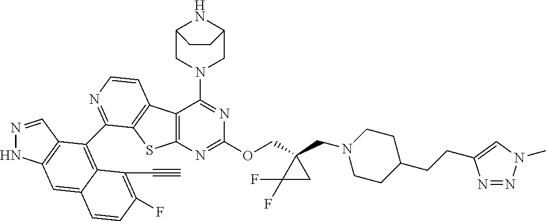 [SMILES: C#Cc1c(F)ccc2cc3[nH]ncc3c(-c3nccc4c3sc3nc(OC[C@]5(CN6CCC(CCc7cn(C)nn7)CC6)CC5(F)F)nc(N5CC6CCC(C5)N6)c34)c12] | 4-((1R,5S)-3,8- diazabicyclo[3.2.1]octan- 3-yl)-2-(((R)-2,2- difluoro-1-((4-(2-(1- methyl-1H-1,2,3- triazol-4- yl)ethyl)piperidin-1- yl)methyl)cyclopropyl) methoxy)-8-(5- ethynyl-6-fluoro-1H- benzo[f]indazol-4- yl)pyrido[4′,3′:4,5]thie- no[2,3-d]pyrimidine | 818 | Step E: NaH (5 equiv), alcohol (1.1 equiv), THF (0.1 M), rt, 1 h |
| | Int-40 | | | | |
| Ex- 106 | 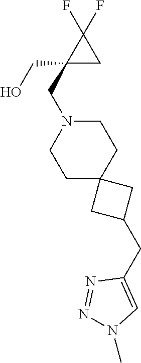 [SMILES: Cn1cc(CC2CC3(CCN(C[C@]4(CO)CC4(F)F)CC3)C2)nn1] | 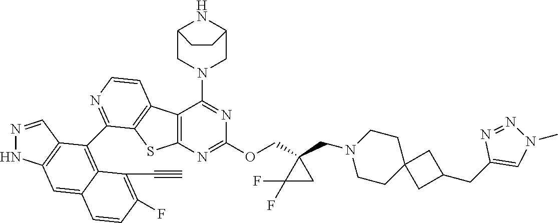 [SMILES: C#Cc1c(F)ccc2cc3[nH]ncc3c(-c3nccc4c3sc3nc(OC[C@]5(CN6CCC7(CC6)CC(Cc6cn(C)nn6)C7)CC5(F)F)nc(N5CC6CCC(C5)N6)c34)c12] | 4-((1R,5S)-3,8- diazabicyclo[3.2.1]octan- 3-yl)-2-(((R)-2,2- difluoro-1-((2-((1- methyl-1H-1,2,3- triazol-4-yl)methyl)-7- azaspiro[3.5]nonan-7- yl)methyl)cyclopropyl) methoxy)-8-(5- ethynyl-6-fluoro-1H- benzo[f]indazol-4- yl)pyrido[4′,3′:4,5]thie- no[2,3-d]pyrimidine | 844 | Step E: NaH (5 equiv), alcohol (1.1 equiv), THF (0.1 M), rt, 1 h |
| | Int-39 | | | | |
| Ex- 107 | 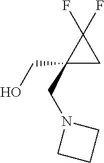 [SMILES: OC[C@@]1(CN2CCC2)CC1(F)F] Int-32 | 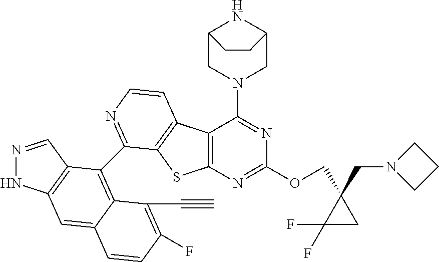 [SMILES: C#Cc1c(F)ccc2cc3[nH]ncc3c(-c3nccc4c3sc3nc(OC[C@]5(CN6CCC6)CC5(F)F)nc(N5CC6CCC(C5)N6)c34)c12] | 2-(((R)-1-(azetidin-1- ylmethyl)-2,2- difluorocyclopropyl)meth- oxy)-4-((1R,5S)- 3,8- diazabicyclo[3.2.1]octan- 3-yl)-8-(5-ethynyl- 6-fluoro-1H- benzo[f]indazol-4- yl)pyrido[4′,3′:4,5]thie- no[2,3-d]pyrimidine | 681 | Step E: NaH (5 equiv), alcohol (1.2 equiv), THF (0.1 M), rt, 1 h |
| Ex- 108 | 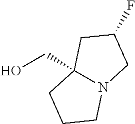 [SMILES: OC[C@@]12CCCN1C[C@@H](F)C2] ((2S,7aS)-2- fluorotetrahydro- 1H- pyrrolizin- 7a(5H)- yl)methanol | 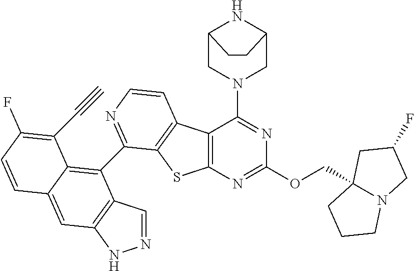 [SMILES: C#Cc1c(F)ccc2cc3[nH]ncc3c(-c3nccc4c3sc3nc(OC[C@@]56CCCN5C[C@@H](F)C6)nc(N5CC6CCC(C5)N6)c34)c12] | 4-(3,8- diazabicyclo[3.2.1]octan- 3-yl)-8-(5-ethynyl- 6-fluoro-1H- benzo[f]indazol-4-yl)- 2-(((2S,7aS)-2- fluorotetrahydro-1H- pyrrolizin-7a(5H)- yl)methoxy)pyrido[4′, 3′:4,5]thieno[2,3- d]pyrimidine | 663 | Step E: LiHMDS (5 equiv), alcohol (1.2 equiv), THF (0.1 M), rt, rt |

### Example 109: ((1R)-1-(((4-((1R,5S)-3,8-diazabicyclo[3.2.1]octan-3-yl)-8-(5-ethynyl-6-fluoro-1H-benzo[f]indazol-4-yl)pyrido[4′,3′:4,5]thieno[2,3-d]pyrimidin-2-yl)ox)methyl)-2,2-difluorocyclopropyl)methanol (Ex-109)

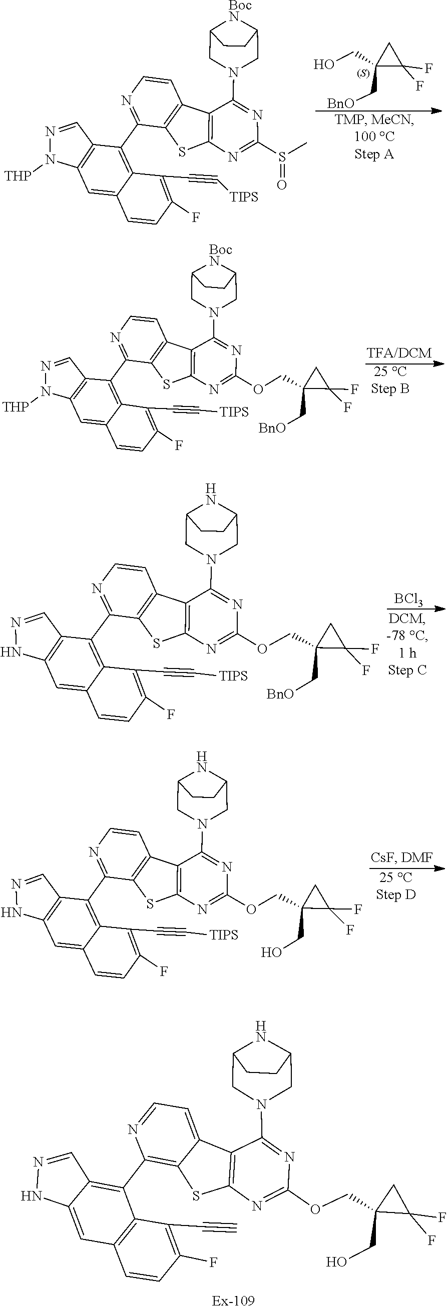

### Step A: tert-butyl (1R,5S)-3-(2-(((R)-1-((benzyloxy)methyl)-2,2-difluorocyclopropyl)methoxy)-8-(6-fluoro-1-(tetrahydro-2H-pyran-2-yl)-5-((triisopropylsilyl)ethynyl)-1H-benzo[f]indazol-4-yl)pyrido[4′,3′:4,5]thieno[2,3-d]pyrimidin-4-yl)-3,8-diazabicyclo[3.2.1]octane-8-carboxylate

To a solution of tert-butyl (1R,5S)-3-(8-(6-fluoro-1-(tetrahydro-2H-pyran-2-yl)-5-((triisopropylsilyl)ethynyl)-1H-benzo[f]indazol-4-yl)-2-(methylsulfinyl)pyrido[4′,3′:4,5]thieno[2,3-d]pyrimidin-4-yl)-3,8-diazabicyclo[3.2.1]octane-8-carboxylate (60 mg, 0.066 mmol) in MeCN (0.5 mL) was added (S)-(1-((benzyloxy)methyl)-2,2-difluorocyclopropyl)methanol (23 mg, 0.099 mmol) and 2,2,6,6-tetramethylpiperidine (187 mg, 1.32 mmol) at 25° C. The reaction was stirred at 100° C. for 16 h under nitrogen atmosphere. The mixture was cooled to rt, diluted with water (~1 mL), and extracted with EtOAc (2×15 mL). The combined organic layers were dried with Na₂SO₄, filtered, and concentrated under reduced pressure. The reaction was purified by prep-TLC (Pet. ether:EtOAc=2:1) to give tert-butyl (1R,5S)-3-(2-(((R)-1-((benzyloxy)methyl)-2,2-difluorocyclopropyl)methoxy)-8-(6-fluoro-1-(tetrahydro-2H-pyran-2-yl)-5-((triisopropylsilyl)ethynyl)-1H-benzo[f]indazol-4-yl)pyrido[4′,3′:4,5]thieno[2,3-d]pyrimidin-4-yl)-3,8-diazabicyclo[3.2.1]octane-8-carboxylate. MS (ESI) [M/2+H]⁺: m/z 537.

### Step B: 2-(((R)-1-((benzyloxy)methyl)-2,2-difluorocyclopropyl)methoxy)-4-((1R,5S)-3,8-diazabicyclo[3.2.1]octan-3-yl)-8-(6-fluoro-5-((triisopropylsilyl)ethynyl)-1H-benzo[f]indazol-4-yl)pyrido[4′,3′:4,5]thieno[2,3-d]pyrimidine

To a solution of TFA (0.20 mL, 2.6 mmol) in DCM (0.2 mL) was added tert-butyl (1R,5S)-3-(2-(((R)-1-((benzyloxy)methyl)-2,2-difluorocyclopropyl)methoxy)-8-(6-fluoro-1-(tetrahydro-2H-pyran-2-yl)-5-((triisopropylsilyl)ethynyl)-1H-benzo[f]indazol-4-yl)pyrido[4′,3′:4,5]thieno[2,3-d]pyrimidin-4-yl)-3,8-diazabicyclo[3.2.1]octane-8-carboxylate (50 mg, 0.047 mmol) at 25° C. Then the reaction was stirred at 25° C. for 0.5 h. Most of the solvent was concentrated in vacuo, the residue was diluted with saturated aqueous NaHCO₃ (2 mL), extracted with DCM (2 mL×3), and the combined organic layers were dried over Na₂SO₄, filtered and concentrated in vacuo to give 2-(((R)-1-((benzyloxy)methyl)-2,2-difluorocyclopropyl)methoxy)-4-((1R,5S)-3,8-diazabicyclo[3.2.1]octan-3-yl)-8-(6-fluoro-5-((triisopropylsilyl)ethynyl)-1H-benzo[f]indazol-4-yl)pyrido[4′,3′:4,5]thieno[2,3-d]pyrimidine. MS (ESI) [M+H]⁺: m/z 888.

### Step C: ((1R)-1-(((4-((1R,5S)-3,8-diazabicyclo[3.2.1]octan-3-yl)-8-(6-fluoro-5-((triisopropylsilyl)ethynyl)-1H-benzo[f]indazol-4-yl)pyrido[4′,3′:4,5]thieno[2,3-d]pyrimidin-2-yl)oxy)methyl)-2,2-difluorocyclopropyl)methanol

To a solution of 2-(((R)-1-((benzyloxy)methyl)-2,2-difluorocyclopropyl)methoxy)-4-((1R,5S)-3,8-diazabicyclo[3.2.1]octan-3-yl)-8-(6-fluoro-5-((triisopropylsilyl)ethynyl)-1H-benzo[f]indazol-4-yl)pyrido[4′,3′:4,5]thieno[2,3-d]pyrimidine (21 mg, 0.024 mmol) in DCM (0.3 mL) was added boron trichloride solution (0.120 mL, 0.120 mmol, 1 M in DCM) at −78° C. The reaction was stirred at −78° C. for 1 h. The mixture was quenched with a solution of DIEA (0.079 mL, 0.45 mmol) in MeOH (9.75 μL, 0.241 mmol), and then was concentrated in vacuo. The crude was purified by prep TLC (DCM/MeOH (NH₃)=10:1) to give ((1R)-1-(((4-((1R,5S)-3,8-diazabicyclo[3.2.1]octan-3-yl)-8-(6-fluoro-5-((triisopropylsilyl)ethynyl)-1H-benzo[f]indazol-4-yl)pyrido[4′,3′:4,5]thieno[2,3-d]pyrimidin-2-yl)oxy)methyl)-2,2-difluorocyclopropyl)methanol. MS (ESI) [M+H]⁺: m/z 798.

### Step D: ((1R)-1-(((4-((1R,5S)-3,8-diazabicyclo[3.2.1]octan-3-yl)-8-(5-ethynyl-6-fluoro-1H-benzo[f]indazol-4-yl)pyrido[4′,3′:4,5]thieno[2,3-d]pyrimidin-2-yl)ox)methyl)-2,2-difluorocyclopropyl)methanol (Ex-109)

To a solution of ((1R)-1-(((4-((1R,5S)-3,8-diazabicyclo[3.2.1]octan-3-yl)-8-(6-fluoro-5-((triisopropylsilyl)ethynyl)-1H-benzo[f]indazol-4-yl)pyrido[4′,3′:4,5]thieno[2,3-d]pyrimidin-2-yl)oxy)methyl)-2,2-difluorocyclopropyl)methanol (15 mg, 0.019 mmol) in DMF (0.3 mL) was added CsF (14 mg, 0.094 mmol) at 25° C. Then the reaction was stirred at 25° C. for 1 h. The reaction mixture was filtered and concentrated in vacuo. The crude was purified by preparative HPLC (water/MeCN w/0.2% FA modifier) to give ((1R)-1-(((4-((1R,5S)-3,8-diazabicyclo[3.2.1]octan-3-yl)-8-(5-ethynyl-6-fluoro-1H-benzo[f]indazol-4-yl)pyrido[4′,3′:4,5]thieno[2,3-d]pyrimidin-2-yl)oxy)methyl)-2,2-difluorocyclopropyl)methanol (Ex-109). ¹H NMR (400 MHz, CD₃OD) δ 8.72 (d, J=5.6 Hz, 1H), 8.36 (s, 1H), 8.23 (dd, J=9.5, 5.9 Hz, 1H), 7.90 (d, J=5.6 Hz, 1H), 7.78 (s, 1H), 7.41 (t, J=8.9 Hz, 1H), 4.69-4.47 (m, 4H), 4.26 (br d, J=12.6 Hz, 1H), 3.89 (br s, 2H), 3.76 (s, 2H), 3.72 (br d, J=13.2 Hz, 1H), 3.30 (br s, 1H), 2.08-1.80 (m, 4H), 1.62-1.42 (m, 2H). MS (ESI) [M+H]⁺: m/z 642.

### Example 110: 4-((1R,5S)-3,8-diazabicyclo[3.2.1]octan-3-yl)-8-(5-ethynyl-6-fluoro-1H-benzo[f]indazol-4-yl)-2-((4-(trifluoromethyl)tetrahydro-2H-pyran-4-yl)methoxy)pyrido[4′,3′:4,5]thieno[2,3-d]pyrimidine (Ex-110)

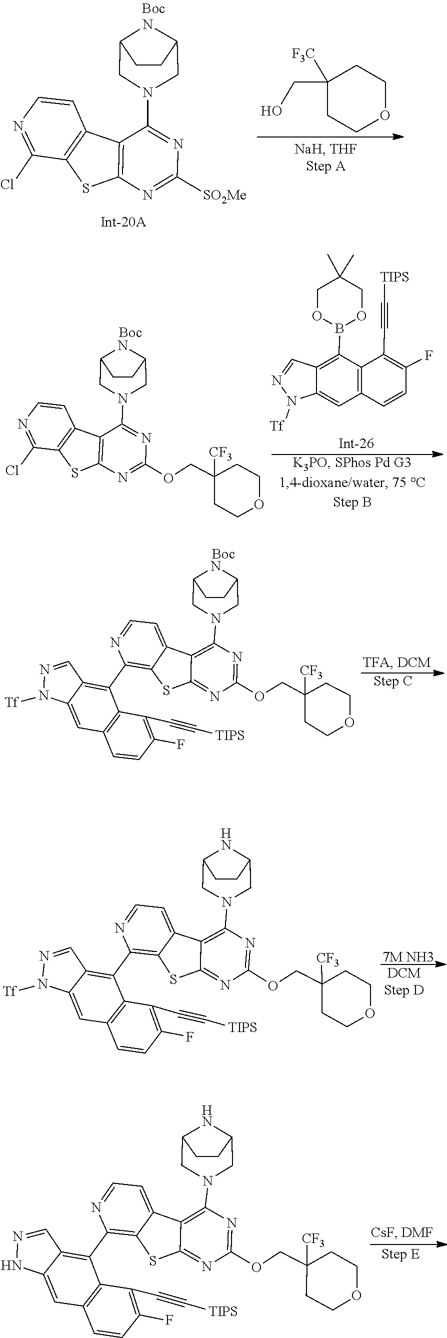

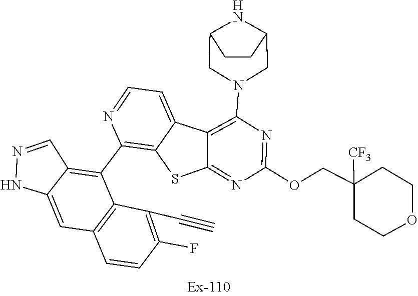 [SMILES: C#Cc1c(F)ccc2cc3[nH]ncc3c(-c3nccc4c3sc3nc(OCC5(C(F)(F)F)CCOCC5)nc(N5CC6CCC(C5)N6)c34)c12]

### Step A: tert-butyl (1R,5S)-3-(8-chloro-2-((4-(trifluoromethyl)tetrahydro-2H-pyran-4-yl)methoxy)pyrido[4′,3′:4,5]thieno[2,3-d]pyrimidin-4-yl)-3,8-diazabicyclo[3.2.1]octane-8-carboxylate

To a mixture of tert-butyl (1R,5S)-3-(8-chloro-2-(methylsulfonyl)pyrido[4′,3′:4,5]thieno[2,3-d]pyrimidin-4-yl)-3,8-diazabicyclo[3.2.1]octane-8-carboxylate (Int-20A) (480 mg, 0.941 mmol), (4-(trifluoromethyl)tetrahydro-2H-pyran-4-yl)methanol (260 mg, 1.41 mmol) and NaH (75 mg, 1.9 mmol, 60% in mineral oil) was added THF (12 mL) under nitrogen. The mixture was stirred at RT for 4 h. Reaction mixture was quenched with MeOH and diluted with EtOAc. The combined extracts were washed with brine solution and concentrated to get tert-butyl (1R,5S)-3-(8-chloro-2-((4-(trifluoromethyl)tetrahydro-2H-pyran-4-yl)methoxy)pyrido[4′,3′:4,5]thieno[2,3-d]pyrimidin-4-yl)-3,8-diazabicyclo[3.2.1]octane-8-carboxylate. MS (ESI) [M+H]⁺: m/z 614.

### Step B: tert-butyl (1R,5S)-3-(8-(6-fluoro-1-((trifluoromethyl)sulfonyl)-5-((triisopropylsilyl)ethynyl)-1H-benzo[f]indazol-4-yl)-2-((4-(trifluoromethyl)tetrahydro-2H-pyran-4-yl)methoxy)pyrido[4′,3′:4,5]thieno[2,3-d]pyrimidin-4-yl)-3,8-diazabicyclo[3.2.1]octane-8-carboxylate

A mixture of tert-butyl (1R,5S)-3-(8-chloro-2-((4-(trifluoromethyl)tetrahydro-2H-pyran-4-yl)methoxy)pyrido[4′,3′:4,5]thieno[2,3-d]pyrimidin-4-yl)-3,8-diazabicyclo[3.2.1]octane-8-carboxylate (250 mg, 0.407 mmol), 4-(5,5-dimethyl-1,3,2-dioxaborinan-2-yl)-6-fluoro-1-((trifluoromethyl)sulfonyl)-5-((triisopropylsilyl)ethynyl)-1H-benzo[f]indazole (Int-26) (373 mg, 0.611 mmol), potassium phosphate tribasic (345 mg, 1.63 mmol) and SPhos Pd G3 (159 mg, 0.204 mmol) was taken in 1,4-dioxane (3 mL) and water (0.5 mL), degassed with vacuum and nitrogen. The reaction mixture was heated at 75° C. for 4 h. The reaction mixture was cooled to RT, diluted with EtOAc, washed with brine solution and concentrated. The product was purified by silica gel column (0-45% of EtOAc in hexane) to afford tert-butyl (1R,5S)-3-(8-(6-fluoro-1-((trifluoromethyl)sulfonyl)-5-((triisopropylsilyl)ethynyl)-1H-benzo[f]indazol-4-yl)-2-((4-(trifluoromethyl)tetrahydro-2H-pyran-4-yl)methoxy)pyrido[4′,3′:4,5]thieno[2,3-d]pyrimidin-4-yl)-3,8-diazabicyclo[3.2.1]octane-8-carboxylate. MS (ESI) [M+H]⁺: m/z 1076.

### Step C: 4-((1R,5S)-3,8-diazabicyclo[3.2.1]octan-3-yl)-8-(6-fluoro-1-((trifluoromethyl)sulfonyl)-5-((triisopropylsilyl)ethynyl)-1H-benzo[f]indazol-4-yl)-2-((4-(trifluoromethyl)tetrahydro-2H-pyran-4-yl)methoxy)pyrido[4′,3′:4,5]thieno[2,3-d]pyrimidine

A mixture of tert-butyl (1R,5S)-3-(8-(6-fluoro-1-((trifluoromethyl)sulfonyl)-5-((triisopropylsilyl)ethynyl)-1H-benzo[f]indazol-4-yl)-2-((4-(trifluoromethyl)tetrahydro-2H-pyran-4-yl)methoxy)pyrido[4′,3′:4,5]thieno[2,3-d]pyrimidin-4-yl)-3,8-diazabicyclo[3.2.1]octane-8-carboxylate (51 mg, 0.047 mmol)) and TFA (0.5 mL) in DCM (1 mL) was stirred at rt for 1 h. The reaction mixture was neutralized with sat. aq. NaHCO₃ solution, extracted with DCM, and the combined organic layers were washed with brine solution and concentrated to get 4-((1R,5S)-3,8-diazabicyclo[3.2.1]octan-3-yl)-8-(6-fluoro-1-((trifluoromethyl)sulfonyl)-5-((triisopropylsilyl)ethynyl)-1H-benzo[f]indazol-4-yl)-2-((4-(trifluoromethyl)tetrahydro-2H-pyran-4-yl)methoxy)pyrido[4′,3′:4,5]thieno[2,3-d]pyrimidine. MS (ESI) [M+H]⁺: m/z 976.

### Step D: 4-((1R,5S)-3,8-diazabicyclo[3.2.1]octan-3-yl)-8-(6-fluoro-5-((triisopropylsilyl) ethynyl)-1H-benzo[f]indazol-4-yl)-2-((4-(trifluoromethyl)tetrahydro-2H-pyran-4-yl)methoxy)pyrido[4′,3′:4,5]thieno[2,3-d]pyrimidine

A mixture of 4-((1R,5S)-3,8-diazabicyclo[3.2.1]octan-3-yl)-8-(6-fluoro-1-((trifluoromethyl)sulfonyl)-5-((triisopropylsilyl)ethynyl)-1H-benzo[f]indazol-4-yl)-2-((4-(trifluoromethyl)tetrahydro-2H-pyran-4-yl)methoxy)pyrido[4′,3′:4,5]thieno[2,3-d]pyrimidine (49 mg, 0.050 mmol), ammonia solution (1.08 mL, 7.53 mmol, 7 M in MeOH) and DCM (0.5 mL) was stirred at RT overnight. The mixture was concentrated, and the filtrate was diluted with EtOAc. The organic layers were washed with brine solution and concentrated to get 4-((1R,5S)-3,8-diazabicyclo[3.2.1]octan-3-yl)-8-(6-fluoro-5-((triisopropylsilyl)ethynyl)-1H-benzo[f]indazol-4-yl)-2-((4-(trifluoromethyl)tetrahydro-2H-pyran-4-yl)methoxy)pyrido[4′,3′:4,5]thieno[2,3-d]pyrimidine. MS (ESI) [M+H]⁺: m/z 844.

### Step E: 4-((1R,5S)-3,8-diazabicyclo[3.2.1]octan-3-yl)-8-(5-ethynyl-6-fluoro-1H-benzo[f]indazol-4-yl)-2-((4-(trifluoromethyl)tetrahydro-2H-pyran-4-yl)methoxy)pyrido[4′,3′:4,5]thieno[2,3-d]pyrimidine (Ex-110)

A mixture of 4-((1R,5S)-3,8-diazabicyclo[3.2.1]octan-3-yl)-8-(6-fluoro-5-((triisopropylsilyl)ethynyl)-1H-benzo[f]indazol-4-yl)-2-((4-(trifluoromethyl)tetrahydro-2H-pyran-4-yl)methoxy)pyrido[4′,3′:4,5]thieno[2,3-d]pyrimidine (40 mg, 0.047 mmol) and CsF (36.0 mg, 0.237 mmol) in DMF (0.5 mL) was stirred at RT for overnight. The reaction mixture was quenched with water, extracted with EtOAc, and the combined extracts were washed with brine solution and concentrated. The product was purified by HPLC (water/MeCN w/NH₄OH modifier) to get 4-((1R,5S)-3,8-diazabicyclo[3.2.1]octan-3-yl)-8-(5-ethynyl-6-fluoro-1H-benzo[f]indazol-4-yl)-2-((4-(trifluoromethyl)tetrahydro-2H-pyran-4-yl)methoxy)pyrido[4′,3′:4,5]thieno[2,3-d]pyrimidine (Ex-110). ¹H NMR (600 MHz, DMSO) δ 13.55 (s, 2H), 8.73 (d, J=5.5 Hz, 1H), 8.44 (s, 1H), 8.37 (dd, J=9.4, 6.1 Hz, 1H), 7.89-7.71 (m, 2H), 7.54 (t, J=9.0 Hz, 1H), 4.71 (s, 2H), 4.29 (d, J=11.6 Hz, 1H), 3.97 (d, J=11.4 Hz, 1H), 3.82-3.66 (m, 4H), 3.65-3.47 (m, 5H), 1.85-1.68 (m, 5H), 1.68-1.48 (m, 3H). MS (ESI) [M+H]⁺: m/z 688.

### Example 111: 4-((1R,5S)-3,8-diazabicyclo[3.2.1]octan-3-yl)-2-(((R)-2,2-difluoro-1-((4-(methoxymethyl)piperidin-1-yl)methyl)cyclopropyl)methoxy)-8-(5-ethynyl-6-fluoro-1H-benzo[f]indazol-4-yl)pyrido[4′,3′:4,5]thieno[2,3-d]pyrimidine (Ex-111)

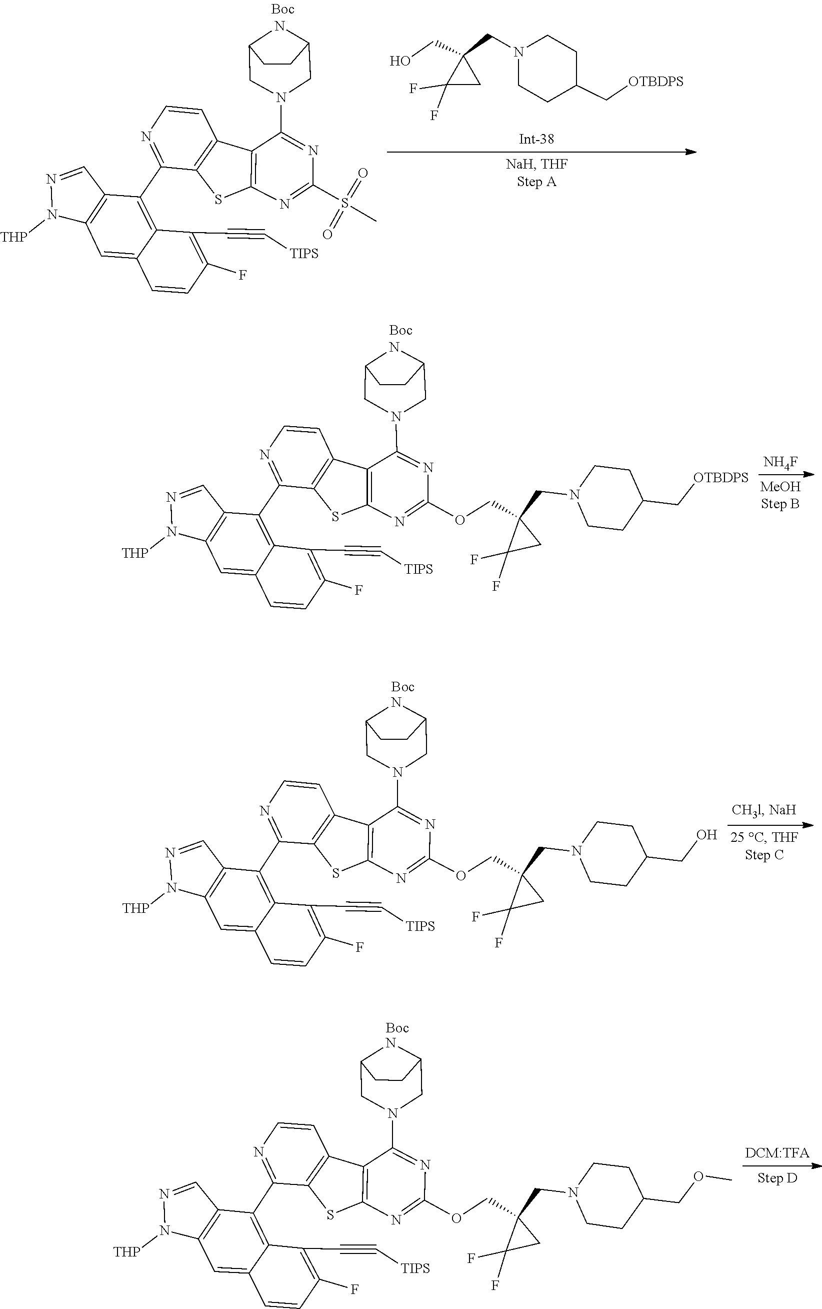

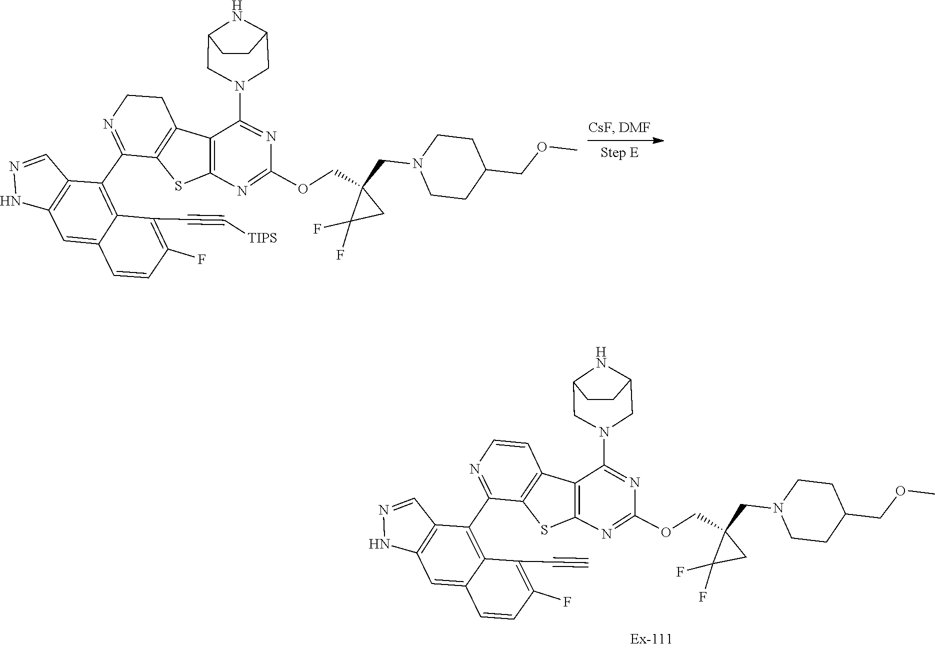

### Step A: tert-butyl (1R,5S)-3-(2-(((R)-1-((4-(((tert-butyldiphenylsilyl)oxy)methyl)piperidin-1-yl)methyl)-2,2-difluorocyclopropyl)methoxy)-8-(6-fluoro-1-(tetrahydro-2H-pyran-2-yl)-5-((triisopropylsilyl)ethynyl)-1H-benzo[f]indazol-4-yl)pyrido[4′,3′:4,5]thieno[2,3-d]pyrimidin-4-yl)-3,8-diazabicyclo[3.2.1]octane-8-carboxylate

To a solution of tert-butyl (1R,5S)-3-(8-(6-fluoro-1-(tetrahydro-2H-pyran-2-yl)-5-((triisopropylsilyl)ethynyl)-1H-benzo[f]indazol-4-yl)-2-(methylsulfonyl)pyrido[4′,3′:4,5]thieno[2,3-d]pyrimidin-4-yl)-3,8-diazabicyclo[3.2.1]octane-8-carboxylate (200 mg, 0.216 mmol) and (R)-(1-((4-(((tert-butyldiphenylsilyl)oxy)methyl)piperidin-1-yl)methyl)-2,2-difluorocyclopropyl)methanol (Int-38) (108 mg, 0.227 mmol) in THF (2 mL) was added sodium hydride (26.0 mg, 1.08 mmol, 60% in mineral oil) at 25° C. The mixture was stirred at 25° C. for 1 h. The mixture was quenched with saturated NH₄Cl solution (4 mL), extracted with EtOAc (10 mL), dried over Na₂SO₄, filtered, and the filtrate was concentrated under reduced pressure. The product was purified by flash silica gel chromatography (5% MeOH in DCM) to give tert-butyl (1R,5S)-3-(2-(((R)-1-((4-(((tert-butyldiphenylsilyl)oxy)methyl)piperidin-1-yl)methyl)-2,2-difluorocyclopropyl)methoxy)-8-(6-fluoro-1-(tetrahydro-2H-pyran-2-yl)-5-((triisopropylsilyl)ethynyl)-1H-benzo[f]indazol-4-yl)pyrido[4′,3′:4,5]thieno[2,3-d]pyrimidin-4-yl)-3,8-diazabicyclo[3.2.1]octane-8-carboxylate. MS (ESI) [M/2+H]⁺: m/z 660.

### Step B: tert-butyl (1R,5S)-3-(2-(((R)-2,2-difluoro-1-((4-(hydroxymethyl)piperidin-1-yl)methyl)cyclopropyl)methoxy)-8-(6-fluoro-1-(tetrahydro-2H-pyran-2-yl)-5-((triisopropylsilyl)ethynyl)-1H-benzo[f]indazol-4-yl)pyrido[4′,3′:4,5]thieno[2,3-d]pyrimidin-4-yl)-3,8-diazabicyclo[3.2.1]octane-8-carboxylate

To a solution of tert-butyl (1R,5S)-3-(2-(((R)-1-((4-(((tert-butyldiphenylsilyl)oxy)methyl)piperidin-1-yl)methyl)-2,2-difluorocyclopropyl)methoxy)-8-(6-fluoro-1-(tetrahydro-2H-pyran-2-yl)-5-((triisopropylsilyl)ethynyl)-1H-benzo[f]indazol-4-yl)pyrido[4′,3′:4,5]thieno[2,3-d]pyrimidin-4-yl)-3,8-diazabicyclo[3.2.1]octane-8-carboxylate (260 mg, 0.197 mmol) in MeOH (3 mL) was added ammonium fluoride (73.1 mg, 1.97 mmol) at 25° C. The mixture was stirred at 50° C. for 15 h. The mixture was cooled, and the solvent was evaporated under reduced pressure. The product was purified by flash silica gel chromatography (5% MeOH in DCM) to give tert-butyl (1R,5S)-3-(2-(((R)-2,2-difluoro-1-((4-(hydroxymethyl)piperidin-1-yl)methyl)cyclopropyl)methoxy)-8-(6-fluoro-1-(tetrahydro-2H-pyran-2-yl)-5-((triisopropylsilyl)ethynyl)-1H-benzo[f]indazol-4-yl)pyrido[4′,3′:4,5]thieno[2,3-d]pyrimidin-4-yl)-3,8-diazabicyclo[3.2.1]octane-8-carboxylate. MS (ESI) [M/2+H]⁺: m/z 541.

### Step C: tert-butyl (1R,5S)-3-(2-(((R)-2,2-difluoro-1-((4-(methoxymethyl)piperidin-1-yl)methyl)cyclopropyl)methoxy)-8-(6-fluoro-1-(tetrahydro-2H-pyran-2-yl)-5-((triisopropylsilyl)ethynyl)-1H-benzo[f]indazol-4-yl)pyrido[4′,3′:4,5]thieno[2,3-d]pyrimidin-4-yl)-3,8-diazabicyclo[3.2.1]octane-8-carboxylate

To a solution of tert-butyl (1R,5S)-3-(2-(((R)-2,2-difluoro-1-((4-(hydroxymethyl)piperidin-1-yl)methyl)cyclopropyl)methoxy)-8-(6-fluoro-1-(tetrahydro-2H-pyran-2-yl)-5-((triisopropylsilyl)ethynyl)-1H-benzo[f]indazol-4-yl)pyrido[4′,3′:4,5]thieno[2,3-d]pyrimidin-4-yl)-3,8-diazabicyclo[3.2.1]octane-8-carboxylate (18 mg, 0.017 mmol) in THF (0.3 mL) was added NaH (0.80 mg, 0.020 mmol, 60% in mineral oil) at 25° C. under N₂ atmosphere and stirred at 25° C. for 10 min. To the mixture was added a solution of iodomethane (3.1 mg, 0.022 mmol) in THF (0.2 mL), and the resulting mixture was stirred at 25° C. for 3 h. The reaction was quenched with saturated aqueous NH₄Cl (1 mL), extracted with EtOAc (2 mL×3), and the organic layer was dried over Na₂SO₄, filtered and concentrated in vacuo. The residue was purified by preparative TLC (Pet. ether:THF=2:3) to give tert-butyl (1R,5S)-3-(2-(((R)-2,2-difluoro-1-((4-(methoxymethyl)piperidin-1-yl)methyl)cyclopropyl)methoxy)-8-(6-fluoro-1-(tetrahydro-2H-pyran-2-yl)-5-((triisopropylsilyl)ethynyl)-1H-benzo[f]indazol-4-yl)pyrido[4′,3′:4,5]thieno[2,3-d]pyrimidin-4-yl)-3,8-diazabicyclo[3.2.1]octane-8-carboxylate. MS (ESI) [M/2+H]⁺: m/z 547.

### Step D: 4-((1R,5S)-3,8-diazabicyclo[3.2.1]octan-3-yl)-2-(((R)-2,2-difluoro-1-((4-(methoxymethyl)piperidin-1-yl)methyl)cyclopropyl)methoxy)-8-(6-fluoro-5-((triisopropylsilyl)ethynyl)-1H-benzo[f]indazol-4-yl)pyrido[4′,3′:4,5]thieno[2,3-d]pyrimidine

Tert-butyl (1R,5S)-3-(2-(((R)-2,2-difluoro-1-((4-(methoxymethyl)piperidin-1-yl)methyl)cyclopropyl)methoxy)-8-(6-fluoro-1-(tetrahydro-2H-pyran-2-yl)-5-((triisopropylsilyl)ethynyl)-1H-benzo[f]indazol-4-yl)pyrido[4′,3′:4,5]thieno[2,3-d]pyrimidin-4-yl)-3,8-diazabicyclo[3.2.1]octane-8-carboxylate (16 mg, 0.015 mmol) was added to a solution of TFA (0.25 mL, 3.24 mmol) in DCM (0.25 mL) at 25° C., and the resulting mixture was stirred at 25° C. for 30 min. The solvent was removed by nitrogen flow, and the residue was basified with saturated aqueous NaHCO₃ (1 mL), extracted with DCM (2 mL×3), and the organic layer was dried over Na₂SO₄, filtered and concentrated in vacuo to give 4-((1R,5S)-3,8-diazabicyclo[3.2.1]octan-3-yl)-2-(((R)-2,2-difluoro-1-((4-(methoxymethyl)piperidin-1-yl)methyl)cyclopropyl)methoxy)-8-(6-fluoro-5-((triisopropylsilyl)ethynyl)-1H-benzo[f]indazol-4-yl)pyrido[4′,3′:4,5]thieno[2,3-d]pyrimidine. MS (ESI) [M+H]⁺: m/z 910.

### Step E: 4-((1R,5S)-3,8-diazabicyclo[3.2.1]octan-3-yl)-2-(((R)-2,2-difluoro-1-((4-(methoxymethyl)piperidin-1-yl)methyl)cyclopropyl)methoxy)-8-(5-ethynyl-6-fluoro-1H-benzo[f]indazol-4-yl)pyrido[4′,3′:4,5]thieno[2,3-d]pyrimidine (Ex-111)

To a solution of 4-((1R,5S)-3,8-diazabicyclo[3.2.1]octan-3-yl)-2-(((R)-2,2-difluoro-1-((4-(methoxymethyl)piperidin-1-yl)methyl)cyclopropyl)methoxy)-8-(6-fluoro-5-((triisopropylsilyl)ethynyl)-1H-benzo[f]indazol-4-yl)pyrido[4′,3′:4,5]thieno[2,3-d]pyrimidine (13 mg, 0.014 mmol) in DMF (0.5 mL) was added CsF (21.7 mg, 0.143 mmol) at 25° C. The mixture was stirred at 25° C. for 1.5 h. The mixture was filtered, and the filtrate was purified by reverse preparative HPLC (16-46% water/MeCN w/0.2% FA modifier) to give 4-((1R,5S)-3,8-diazabicyclo[3.2.1]octan-3-yl)-2-(((R)-2,2-difluoro-1-((4-(methoxymethyl)piperidin-1-yl)methyl)cyclopropyl)methoxy)-8-(5-ethynyl-6-fluoro-1H-benzo[f]indazol-4-yl)pyrido[4′,3′:4,5]thieno[2,3-d]pyrimidine (Ex-111). ¹H NMR (400 MHz, CD₃OD) δ 8.59-8.66 (m, 1H), 8.26 (s, 1H), 8.14 (dd, J=5.8, 9.4 Hz, 1H), 7.76-7.85 (m, 1H), 7.68 (s, 1H), 7.31 (t, J=8.9 Hz, 1H), 4.50 (br d, J=11.0 Hz, 4H), 4.10-4.23 (m, 2H), 3.77 (br d, J=14.8 Hz, 3H), 3.61 (br d, J=13.8 Hz, 1H), 3.24 (s, 3H), 3.15 (d, J=1.7 Hz, 2H), 2.94-3.06 (m, 2H), 2.78-2.86 (in, TH), 2.72 (br d, J=11.4 Hz, TH), 2.40 (br t, J=13.8 Hz, TH), 2.09 (br t, J=7.6 Hz, TH), 1.94 (br t, J=6.1 Hz, 3H), 1.75-1.86 (m, 4H), 1.51 (br d, J=8.7 Hz, 3H). MS (ESI) [M+H]⁺: m/z 753.

### Examples 112 and 113: 1-((1R,5S)-8-(2-(((2R,7aS)-2-fluorotetrahydro-TH-pyrrolizin-7a(5H)-yl)methoxy)-8-(6-methyl-5-(trifluoromethyl)-TH-indazol-4-yl)pyrido[4′,3′:4,5]thieno[2,3-d]pyrimidin-4-yl)-3,8-diazabicyclo[3.2.1]octan-3-yl)propan-2-ol (Ex-112) and 1-(1R,5S)-3-(2-(((2R,7aS)-2-fluorotetrahydro-1H-pyrrolizin-7a(5H)-yl)methoxy)-8-(6-methyl-5-(trifluoromethyl)-1H-indazol-4-yl)pyrido[4′,3′:4,5]thieno[2,3-d]pyrimidin-4-yl)-3,8-diazabicyclo[3.2.1]octan-8-yl)propan-2-ol (Ex-113)

Examples 112 and 113 in the table below were synthesized using a similar procedure as described in the synthesis of Ex-65 by making the appropriate substitutions for starting material, intermediates, and/or reagents. Appropriate substitutions are available commercially, synthesized as described in the literature, synthesized using methods available to those skilled in the art, or synthesized as described herein.

| | | | | [M + H]⁺ |
|---|---|---|---|---|
| Ex. | Starting material | Structure | Compound Name | Found |
| Ex-112 | 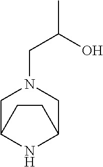 Int-41-1 | 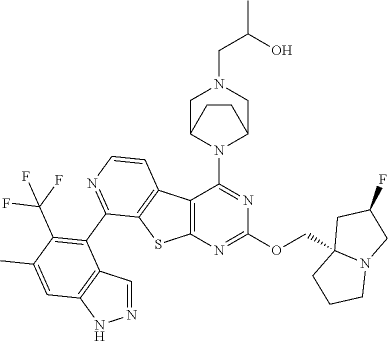 [SMILES: Cc1cc2[nH]ncc2c(-c2nccc3c2sc2nc(OC[C@@]45CCCN4C[C@H](F)C5)nc(N4C5CCC4CN(CC(C)O)C5)c23)c1C(F)(F)F] | 1-((1R,5S)-8-(2- (((2R,7aS)-2- fluorotetrahydro-1H- pyrrolizin-7a(5H)- yl)methoxy)-8-(6- methyl-5- (trifluoromethyl)-1H- indazol-4- yl)pyrido[4′,3′:4,5]thieno [2,3-d]pyrimidin-4-yl)- 3,8- diazabicyclo[3.2.1]octan- 3-ylpropan-2-ol | 711 |
| Ex-113 | 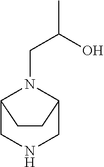 Int-42-1 | 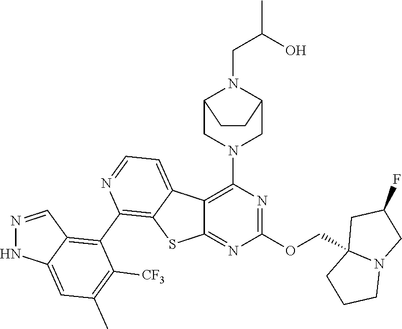 [SMILES: Cc1cc2[nH]ncc2c(-c2nccc3c2sc2nc(OC[C@@]45CCCN4C[C@H](F)C5)nc(N4CC5CCC(C4)N5CC(C)O)c23)c1C(F)(F)F] | 1-((1R,5S)-3-(2- (((2R,7aS)-2- fluorotetrahydro-1H- pyrrolizin-7a(5H)- yl)methoxy)-8-(6- methyl-5- (trifluoromethyl)-1H- indazol-4- yl)pyrido[4′,3′:4,5]thieno [2,3-d]pyrimidin-4-yl)- 3,8- diazabicyclo[3.2.1]octan- 8-yl)propan-2-ol | 711 |

### Example 114: (1S,5S,6S)-3-(8-(5-ethynyl-6-fluoro-TH-benzo[f]indazol-4-yl)-2-(((S,Z)-2-(fluoromethylene)tetrahydro-TH-pyrrolizin-7a(5H)-yl)methoxy)pyrido[4′,3′:4,5]thieno[2,3-d]pyrimidin-4-yl)-3,8-diazabicyclo[3.2.1]octan-6-ol (Ex-114)

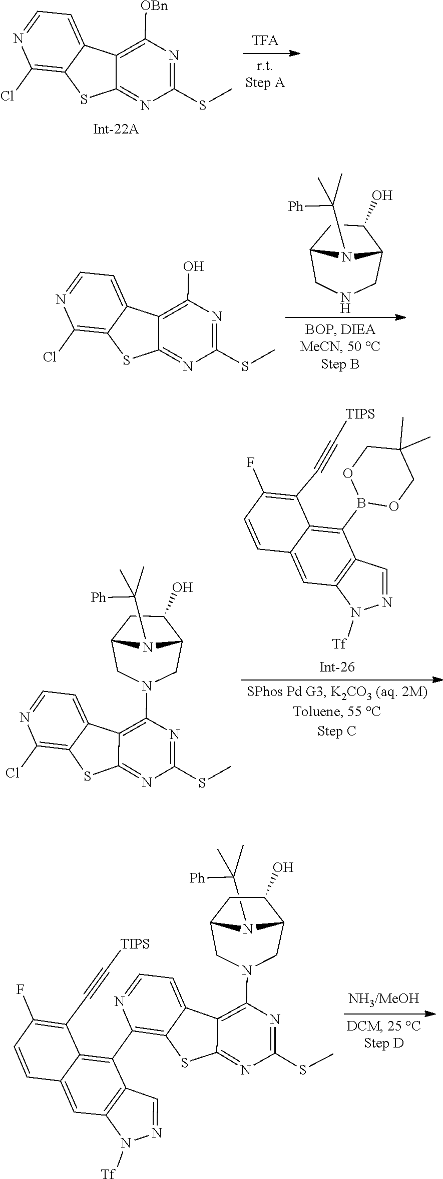

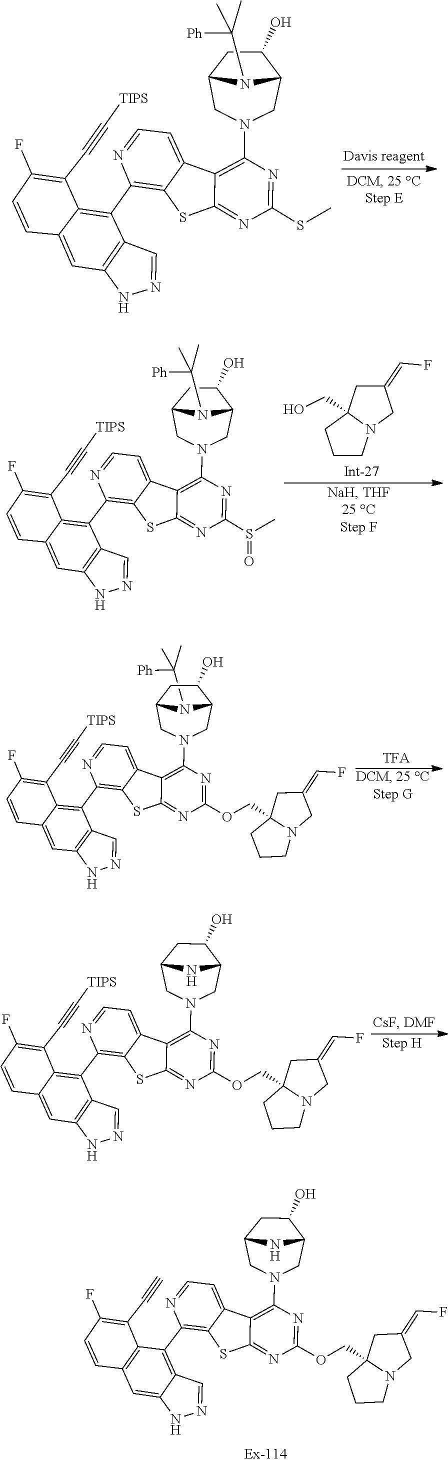

### Step A: 8-chloro-2-(methylthio)pyrido[4′,3′:4,5]thieno[2,3-d]pyrimidin-4-ol

A solution of 4-(benzyloxy)-8-chloro-2-(methylthio)pyrido[4′,3′:4,5]thieno[2,3-d]pyrimidine (Int-22A) (2.00 g, 5.35 mmol) in TFA (20.0 mL, 260 mmol) was stirred at 25° C. for 15 h. The solvent was evaporated under reduced pressure to give 8-chloro-2-(methylthio)pyrido[4′,3′:4,5]thieno[2,3-d]pyrimidin-4-ol as the TFA salt. The material was used without further purification. MS (ESI) [M+H]⁺: m/z 284.

### Step B: (1S,5S,6S)-3-(8-chloro-2-(methylthio)pyrido[4′,3′:4,5]thieno[2,3-d]pyrimidin-4-yl)-8-(2-phenylpropan-2-yl)-3,8-diazabicyclo[3.2.1]octan-6-ol

To a solution of 8-chloro-2-(methylthio)pyrido[4′,3′:4,5]thieno[2,3-d]pyrimidin-4-ol (1.08 g, 2.72 mmol) in MeCN (10 mL) was added BOP (1.80 g, 4.07 mmol) and DIEA (2.37 mL, 13.6 mmol) at 25° C., and the mixture was stirred at 25° C. for 10 min. Then (1S,5S,6S)-8-(2-phenylpropan-2-yl)-3,8-diazabicyclo[3.2.1]octan-6-ol (0.803 g, 3.26 mmol) was added to the reaction solution, and the mixture was stirred at 50° C. for 2 h. The mixture was cooled, and the solvent was evaporated under reduced pressure. The product was purified by flash silica gel chromatography (25% ethyl acetate in petroleum ether gradient) to give (1S,5S,6S)-3-(8-chloro-2-(methylthio)pyrido[4′,3′:4,5]thieno[2,3-d]pyrimidin-4-yl)-8-(2-phenylpropan-2-yl)-3,8-diazabicyclo[3.2.1]octan-6-ol. MS (ESI) [M+H]⁺: m/z 512.

### Step C: (1S,5S,6S)-3-(8-(6-fluoro-1-((trifluoromethyl)sulfonyl)-5-((triisopropylsilyl)ethynyl)-1H-benzo[f]indazol-4-yl)-2-(methylthio)pyrido[4′,3′:4,5]thieno[2,3-d]pyrimidin-4-yl)-8-(2-phenylpropan-2-yl)-3,8-diazabicyclo[3.2.1]octan-6-ol

To a mixture of (1S,5S,6S)-3-(8-chloro-2-(methylthio)pyrido[4′,3′:4,5]thieno[2,3-d]pyrimidin-4-yl)-8-(2-phenylpropan-2-yl)-3,8-diazabicyclo[3.2.1]octan-6-ol (610 mg, 1.19 mmol), 4-(5,5-dimethyl-1,3,2-dioxaborinan-2-yl)-6-fluoro-1-((trifluoromethyl)sulfonyl)-5-((triisopropylsilyl)ethynyl)-1H-benzo[f]indazole (Int-26) (873 mg, 1.43 mmol) and SPhos Pd G3 (465 mg, 0.596 mmol) in toluene (9 mL) was added potassium carbonate (1.79 mL, 3.57 mmol, 2 M in H₂O) at 25° C. The mixture was stirred at 55° C. for 2 h under N₂ atmosphere. The mixture was cooled, diluted with EtOAc (60 mL), washed with H₂O (10 mL), dried over Na₂SO₄, filtered, and the solvent was evaporated under reduced pressure. The product was purified by flash silica gel chromatography (20% ethyl acetate in petroleum ether) to give (1S,5S,6S)-3-(8-(6-fluoro-1-((trifluoromethyl)sulfonyl)-5-((triisopropylsilyl)ethynyl)-1H-benzo[f]indazol-4-yl)-2-(methylthio)pyrido[4′,3′:4,5]thieno[2,3-d]pyrimidin-4-yl)-8-(2-phenylpropan-2-yl)-3,8-diazabicyclo[3.2.1]octan-6-ol. MS (ESI) [M+H]⁺: m/z 974

### Step D: (1S,5S,6S)-3-(8-(6-fluoro-5-((triisopropylsilyl)ethynyl)-1H-benzo[f]indazol-4-yl)-2-(methylthio)pyrido[4′,3′:4,5]thieno[2,3-d]pyrimidin-4-yl)-8-(2-phenylpropan-2-yl)-3,8-diazabicyclo[3.2.1]octan-6-ol

To a solution of (1S,5S,6S)-3-(8-(6-fluoro-1-((trifluoromethyl)sulfonyl)-5-((triisopropylsilyl)ethynyl)-1H-benzo[f]indazol-4-yl)-2-(methylthio)pyrido[4′,3′:4,5]thieno[2,3-d]pyrimidin-4-yl)-8-(2-phenylpropan-2-yl)-3,8-diazabicyclo[3.2.1]octan-6-ol (1.00 g, 1.03 mmol) in DCM (5 mL) was added ammonia solution (10 mL, 70.0 mmol, 7 M in MeOH) at 25° C. The mixture was stirred at 25° C. for 3 h. The solvent was evaporated under reduced pressure. The crude product was purified by flash silica gel chromatography (40% EtOAc in petroleum ether) to give (1S,5S,6S)-3-(8-(6-fluoro-5-((triisopropylsilyl)ethynyl)-1H-benzo[f]indazol-4-yl)-2-(methylthio)pyrido[4′,3′:4,5]thieno[2,3-d]pyrimidin-4-yl)-8-(2-phenylpropan-2-yl)-3,8-diazabicyclo[3.2.1]octan-6-ol. MS (ESI) [M+H]⁺: m/z 842.

### Step E: (1S,5S,6S)-3-(8-(6-fluoro-5-((triisopropylsilyl)ethynyl)-1H-benzo[f]indazol-4-yl)-2-(methylsulfinyl)pyrido[4′,3′:4,5]thieno[2,3-d]pyrimidin-4-yl)-8-(2-phenylpropan-2-yl)-3,8-diazabicyclo[3.2.1]octan-6-ol

To a solution of (1S,5S,6S)-3-(8-(6-fluoro-5-((triisopropylsilyl)ethynyl)-1H-benzo[f]indazol-4-yl)-2-(methylthio)pyrido[4′,3′:4,5]thieno[2,3-d]pyrimidin-4-yl)-8-(2-phenylpropan-2-yl)-3,8-diazabicyclo[3.2.1]octan-6-ol (700 mg, 0.831 mmol) in DCM (7 mL) was added 3-phenyl-2-(phenylsulfonyl)-1,2-oxaziridine (239 mg, 0.914 mmol) at 25° C. The mixture was stirred at 25° C. for 15 h. The solvent was evaporated under reduced pressure. The product was purified by flash silica gel chromatography (5% MeOH in DCM) to give (1S,5S,6S)-3-(8-(6-fluoro-5-((triisopropylsilyl)ethynyl)-1H-benzo[f]indazol-4-yl)-2-(methylsulfinyl)pyrido[4′,3′:4,5]thieno[2,3-d]pyrimidin-4-yl)-8-(2-phenylpropan-2-yl)-3,8-diazabicyclo[3.2.1]octan-6-ol. MS (ESI) [M+H]⁺: m/z 858.

### Step F: (1S,5S,6S)-3-(8-(6-fluoro-5-((triisopropylsilyl)ethynyl)-1H-benzo[f]indazol-4-yl)-2-(((S,Z)-2-(fluoromethylene)tetrahydro-1H-pyrrolizin-7a(5H)-yl)methoxy)pyrido[4′,3′:4,5]thieno[2,3-d]pyrimidin-4-yl)-8-(2-phenylpropan-2-yl)-3,8-diazabicyclo[3.2.1]octan-6-ol

To a solution of (1S,5S,6S)-3-(8-(6-fluoro-5-((triisopropylsilyl)ethynyl)-1H-benzo[f]indazol-4-yl)-2-(methylsulfinyl)pyrido[4′,3′:4,5]thieno[2,3-d]pyrimidin-4-yl)-8-(2-phenylpropan-2-yl)-3,8-diazabicyclo[3.2.1]octan-6-ol (200 mg, 0.233 mmol) and (S,Z)-(2-(fluoromethylene)tetrahydro-1H-pyrrolizin-7a(5H)-yl)methanol (Int-27) (60.0 mg, 0.350 mmol) in THF (3 mL) was added NaH (50.0 mg, 1.25 mmol, 60% Wt) at 25° C. The mixture was stirred at 25° C. for 2 h. The mixture was quenched with saturated aqueous NH₄Cl solution (5 mL), extracted with EtOAc (10 mL), dried over Na₂SO₄, filtered, and the solvent was evaporated under reduced pressure. The residue was purified by preparative TLC (DCM:MeOH=10:1) to give (1S,5S,6S)-3-(8-(6-fluoro-5-((triisopropylsilyl)ethynyl)-1H-benzo[f]indazol-4-yl)-2-(((S,Z)-2-(fluoromethylene)tetrahydro-1H-pyrrolizin-7a(5H)-yl)methoxy)pyrido[4′,3′:4,5]thieno[2,3-d]pyrimidin-4-yl)-8-(2-phenylpropan-2-yl)-3,8-diazabicyclo[3.2.1]octan-6-ol. MS (ESI) [M+H]⁺: m/z 965.

### Step G: (1S,5S,6S)-3-(8-(6-fluoro-5-((triisopropylsilyl)ethynyl)-1H-benzo[f]indazol-4-yl)-2-(((S,Z)-2-(fluoromethylene)tetrahydro-1H-pyrrolizin-7a(5H)-yl)methoxy)pyrido[4′,3′:4,5]thieno[2,3-d]pyrimidin-4-yl)-3,8-diazabicyclo[3.2.1]octan-6-ol

To a solution of (1S,5S,6S)-3-(8-(6-fluoro-5-((triisopropylsilyl)ethynyl)-1H-benzo[f]indazol-4-yl)-2-(((S,Z)-2-(fluoromethylene)tetrahydro-1H-pyrrolizin-7a(5H)-yl)methoxy)pyrido[4′,3′:4,5]thieno[2,3-d]pyrimidin-4-yl)-8-(2-phenylpropan-2-yl)-3,8-diazabicyclo[3.2.1]octan-6-ol (25 mg, 0.026 mmol) in DCM (1 mL) was added TFA (0.20 mL, 0.026 mmol) at 25° C. The mixture was stirred at 25° C. for 3 h. The mixture was cooled, basified with saturated NaHCO₃ solution (4 mL), extracted with DCM (10 mL), dried over Na₂SO₄, filtered, and the solvent was evaporated under reduced pressure to give the residue. The residue was purified by preparative TLC (DCM:MeOH=10:1) to give (1S,5S,6S)-3-(8-(6-fluoro-5-((triisopropylsilyl)ethynyl)-1H-benzo[f]indazol-4-yl)-2-(((S,Z)-2-(fluoromethylene)tetrahydro-1H-pyrrolizin-7a(5H)-yl)methoxy)pyrido[4′,3′:4,5]thieno[2,3-d]pyrimidin-4-yl)-3,8-diazabicyclo[3.2.1]octan-6-ol. MS (ESI) [M+H]⁺: m/z 847.

### Step H: (1S,5S,6S)-3-(8-(5-ethynyl-6-fluoro-1H-benzo[f]indazol-4-yl)-2-(((S,Z)-2-(fluoromethylene)tetrahydro-1H-pyrrolizin-7a(5H)-yl)methoxy)pyrido[4′,3′:4,5]thieno[2,3-d]pyrimidin-4-yl)-3,8-diazabicyclo[3.2.1]octan-6-ol (Ex-114)

To a solution of (1S,5S,6S)-3-(8-(6-fluoro-5-((triisopropylsilyl)ethynyl)-1H-benzo[f]indazol-4-yl)-2-(((S,Z)-2-(fluoromethylene)tetrahydro-1H-pyrrolizin-7a(5H)-yl)methoxy)pyrido[4′,3′:4,5]thieno[2,3-d]pyrimidin-4-yl)-3,8-diazabicyclo[3.2.1]octan-6-ol (15 mg, 0.018 mmol) in DMF (0.2 mL) was added CsF (15 mg, 0.099 mmol) at 25° C. The mixture was stirred at 25° C. for 3 h. The reaction solution was purified by reverse preparative HPLC (MeCN/H₂O w/NH₄HCO₃ modifier) to give (1S,5S,6S)-3-(8-(5-ethynyl-6-fluoro-1H-benzo[f]indazol-4-yl)-2-(((S,Z)-2-(fluoromethylene)tetrahydro-1H-pyrrolizin-7a(5H)-yl)methoxy)pyrido[4′,3′:4,5]thieno[2,3-d]pyrimidin-4-yl)-3,8-diazabicyclo[3.2.1]octan-6-ol (Ex-114). ¹H NMR (400 MHz, CD₃OD) δ 8.69 (dd, J=2.2, 5.5 Hz, 1H), 8.35 (s, 1H), 8.22 (dd, J=6.0, 9.2 Hz, 1H), 8.14-7.97 (m, 1H), 7.82-7.72 (m, 1H), 7.40 (t, J=8.9 Hz, 1H), 6.77-6.47 (m, 1H), 4.83-4.56 (m, 1H), 4.41-4.30 (m, 1H), 4.29-4.08 (m, 3H), 3.99-3.77 (m, 2H), 3.66-3.35 (m, 4H), 3.20 (s, 1H), 3.15-3.06 (m, 1H), 2.70-2.66 (m, 2H), 2.43-2.39 (m, 1H), 2.35-2.21 (m, 1H), 2.14-2.03 (m, 1H), 2.02-1.72 (m, 4H). MS (ESI) [M+H]⁺: m/z 691.

### Example 115: 4′-((1R,5S)-3,8-diazabicyclo[3.2.1]octan-3-yl)-8′-(6-chloro-5-cyclopropyl-1H-indazol-4-yl)-2′-(((2R,7aS)-2-fluorotetrahydro-1H-pyrrolizin-7a(5H)-yl)methoxy)spiro[cyclopropane-1,9′-pyrido[4′,3′:3,4]cyclopenta[1,2-d]pyrimidine] (Ex-115)

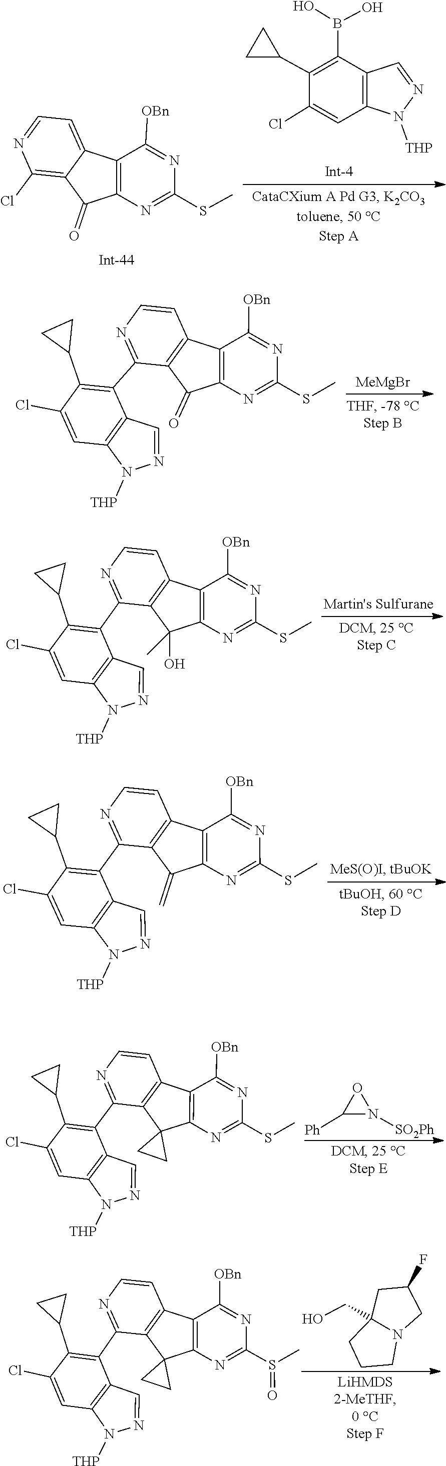

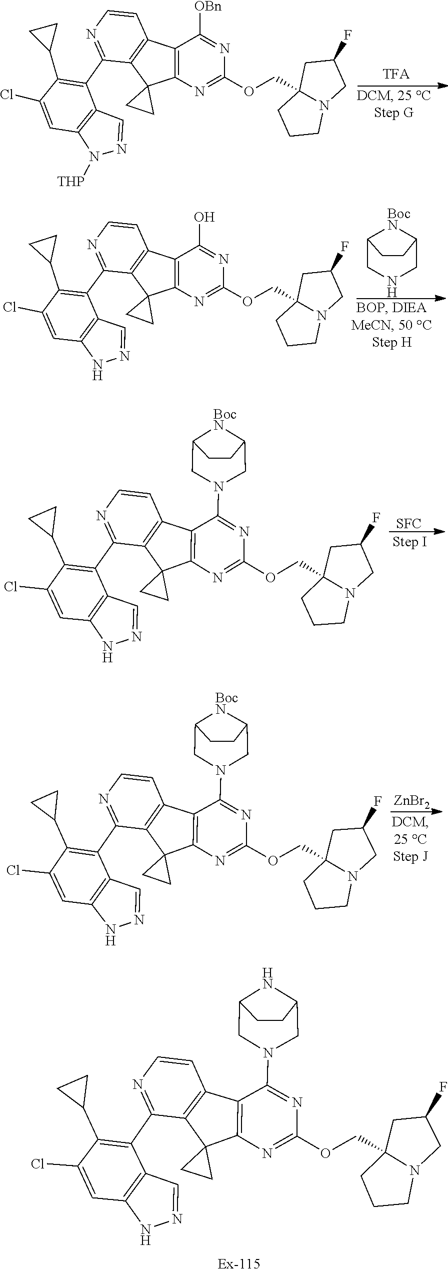

### Step A: 4-(benzyloxy)-8-(6-chloro-5-cyclopropyl-1-(tetrahydro-2H-pyran-2-yl)-1H-indazol-4-yl)-2-(methylthio)-9H-pyrido[4′,3′:3,4]cyclopenta[1,2-d]pyrimidin-9-one

To a solution of 4-(benzyloxy)-8-chloro-2-(methylthio)-9H-pyrido[4′,3′:3,4]cyclopenta[1,2-d]pyrimidin-9-one (Int-44) (5 g, 13.52 mmol) in toluene (60 mL) was added potassium carbonate (20.3 mL, 40.6 mmol, 2M in H₂O), CataCXiumA Pd G3 (4.52 g, 6.76 mmol) and (6-chloro-5-cyclopropyl-1-(tetrahydro-2H-pyran-2-yl)-1H-indazol-4-yl)boronic acid (Int-4) (5.20 g, 16.2 mmol) at 25° C., and the reaction mixture was stirred at 50° C. for 16 h under N₂ atmosphere. The reaction mixture was diluted with H₂O (50 mL), extracted with DCM (80 mL×3), and the organic layer was dried over Na₂SO₄, filtered and concentrated in vacuo. The crude was purified by flash silica gel chromatography (0 to 36% EtOAc in petroleum ether) to give 4-(benzyloxy)-8-(6-chloro-5-cyclopropyl-1-(tetrahydro-2H-pyran-2-yl)-1H-indazol-4-yl)-2-(methylthio)-9H-pyrido[4′,3′:3,4]cyclopenta[1,2-d]pyrimidin-9-one. MS (ESI) [M+H]⁺: m/z 610.

### Step B: 4-(benzyloxy)-8-(6-chloro-5-cyclopropyl-1-(tetrahydro-2H-pyran-2-yl)-1H-indazol-4-yl)-9-methyl-2-(methylthio)-9H-pyrido[4′,3′:3,4]cyclopenta[1,2-d]pyrimidin-9-ol

To a solution of 4-(benzyloxy)-8-(6-chloro-5-cyclopropyl-1-(tetrahydro-2H-pyran-2-yl)-1H-indazol-4-yl)-2-(methylthio)-9H-pyrido[4′,3′:3,4]cyclopenta[1,2-d]pyrimidin-9-one (4.7 g, 7.7 mmol) in THF (50 mL) was added methylmagnesium bromide (5.65 mL, 17.0 mmol, 3M in THF) at −78° C., and the reaction mixture was stirred at −78° C. for 40 min under N₂ atmosphere. The reaction mixture was quenched with sat. aq. NH₄Cl (30 mL), extracted with EtOAc (30 mL×3). The organic layers were washed with brine (3×10 mL), dried over sodium sulflate, filtered and the filtrate was concentrated in vacuo. The residue was purified by flash silica gel chromatography (0 to 45% EtOAc in petroleum ether) to give 4-(benzyloxy)-8-(6-chloro-5-cyclopropyl-1-(tetrahydro-2H-pyran-2-yl)-1H-indazol-4-yl)-9-methyl-2-(methylthio)-9H-pyrido[4′,3′:3,4]cyclopenta[1,2-d]pyrimidin-9-ol. MS (ESI) [M+H]⁺: m/z 626.

### Step C: 4-(benzyloxy)-8-(6-chloro-5-cyclopropyl-1-(tetrahydro-2H-pyran-2-yl)-1H-indazol-4-yl)-9-methylene-2-(methylthio)-9H-pyrido[4′,3′:3,4]cyclopenta[1,2-d]pyrimidine

To a solution of 4-(benzyloxy)-8-(6-chloro-5-cyclopropyl-1-(tetrahydro-2H-pyran-2-yl)-1H-indazol-4-yl)-9-methyl-2-(methylthio)-9H-pyrido[4′,3′:3,4]cyclopenta[1,2-d]pyrimidin-9-ol (3.5 g, 5.6 mmol) in DCM (40 mL) was added Martin's sulfurane (5.64 g, 8.38 mmol) at 25° C., and the reaction mixture was stirred at 25° C. for 20 min under N₂ atmosphere. The reaction mixture was concentrated in vacuo to give a residue which was purified by flash silica gel chromatography (0 to 17% THF in petroleum ether) to give 4-(benzyloxy)-8-(6-chloro-5-cyclopropyl-1-(tetrahydro-2H-pyran-2-yl)-1H-indazol-4-yl)-9-methylene-2-(methylthio)-9H-pyrido[4′,3′:3,4]cyclopenta[1,2-d]pyrimidine. MS (ESI) [M+H]⁺: m/z 608.

### Step D: 4′-(benzyloxy)-8′-(6-chloro-5-cyclopropyl-1-(tetrahydro-2H-pyran-2-yl)-1H-indazol-4-yl)-2′-(methylthio)spiro[cyclopropane-1,9′-pyrido[4′,3′:3,4]cyclopenta[1,2-d]pyrimidine]

To a solution of trimethylsulfoxonium iodide (0.847 g, 3.85 mmol) in t-BuOH (15 mL) was added potassium tert-butoxide (0.432 g, 3.85 mmol) at 25° C. under N₂ atmosphere, and the reaction mixture was stirred at 25° C. for 1 h. Then to the reaction mixture was added a solution of 4-(benzyloxy)-8-(6-chloro-5-cyclopropyl-1-(tetrahydro-2H-pyran-2-yl)-1H-indazol-4-yl)-9-methylene-2-(methylthio)-9H-pyrido[4′,3′:3,4]cyclopenta[1,2-d]pyrimidine (1.80 g, 2.96 mmol) in t-BuOH (15 mL), and the resulting reaction mixture was stirred at 60° C. for 1 h. The reaction was quenched with sat. aq. NH₄Cl (20 mL) and extracted with EtOAc (20 mL×3). The combined organic phase was washed with brine, dried over anhydrous Na₂SO₄, filtered and concentrated in vacuo. The crude was purified by flash silica gel chromatography (0 to 37% THF in petroleum ether) to give 4′-(benzyloxy)-8′-(6-chloro-5-cyclopropyl-1-(tetrahydro-2H-pyran-2-yl)-1H-indazol-4-yl)-2′-(methylthio)spiro[cyclopropane-1,9′-pyrido[4′,3′:3,4]cyclopenta[1,2-d]pyrimidine]. MS (ESI) [M+H]⁺: m/z 622.

### Step E: 4′-(benzyloxy)-8′-(6-chloro-5-cyclopropyl-1-(tetrahydro-2H-pyran-2-yl)-1H-indazol-4-yl)-2′-(methylsulfinyl)spiro[cyclopropane-1,9′-pyrido[4′,3′:3,4]cyclopenta[1,2-d]pyrimidine]

To a solution of 4′-(benzyloxy)-8′-(6-chloro-5-cyclopropyl-1-(tetrahydro-2H-pyran-2-yl)-1H-indazol-4-yl)-2′-(methylthio)spiro[cyclopropane-1,9′-pyrido[4′,3′:3,4]cyclopenta[1,2-d]pyrimidine](1.00 g, 1.61 mmol) in DCM (15 mL) was added 3-phenyl-2-(phenylsulfonyl)-1,2-oxaziridine (1.26 g, 4.82 mmol) at 25° C. under N₂ atmosphere, and the mixture was stirred at 25° C. for 1 h. The mixture was concentrated in vacuo to give a residue which was purified by flash silica gel chromatography (0 to 53% Pet. Ether in EtOAc) to give 4′-(benzyloxy)-8′-(6-chloro-5-cyclopropyl-1-(tetrahydro-2H-pyran-2-yl)-1H-indazol-4-yl)-2′-(methylsulfinyl)spiro[cyclopropane-1,9′-pyrido[4′,3′:3,4]cyclopenta[1,2-d]pyrimidine]. MS (ESI) [M+H]⁺: m/z 638.

### Step F: 4′-(benzyloxy)-8′-(6-chloro-5-cyclopropyl-1-(tetrahydro-2H-pyran-2-yl)-1H-indazol-4-yl)-2′-(((2R,7aS)-2-fluorotetrahydro-1H-pyrrolizin-7a(5H)-yl)methoxy)spiro[cyclopropane-1,9′-pyrido[4′,3′:3,4]cyclopenta[1,2-d]pyrimidine]

To a solution of 4′-(benzyloxy)-8′-(6-chloro-5-cyclopropyl-1-(tetrahydro-2H-pyran-2-yl)-1H-indazol-4-yl)-2′-(methylsulfinyl)spiro[cyclopropane-1,9′-pyrido[4′,3′:3,4]cyclopenta[1,2-d]pyrimidine](450 mg, 0.705 mmol) and ((2R,7aS)-2-fluorotetrahydro-1H-pyrrolizin-7a(5H)-yl)methanol (168 mg, 1.06 mmol) in 2-methyltetrahydrofuran (5 mL) was added LiHMDS (1.41 mL, 1.41 mmol, 1 M in THF) at 0° C. under N₂ atmosphere, and the mixture was stirred at 0° C. for 0.5 h. The reaction was quenched with sat. aq. NH₄Cl (10 mL) and extracted with EtOAc (3×10 mL). The organic layer was washed with brine (2 mL×2), dried over sodium sulfate, filtered, and the filtrate was concentrated in vacuo. The residue was purified by flash silica gel chromatography (0 to 100% Pet. Ether in EtOAc, basified by Et₃N) to give 4′-(benzyloxy)-8′-(6-chloro-5-cyclopropyl-1-(tetrahydro-2H-pyran-2-yl)-1H-indazol-4-yl)-2′-(((2R,7aS)-2-fluorotetrahydro-1H-pyrrolizin-7a(5H)-yl)methoxy)spiro[cyclopropane-1,9′-pyrido[4′,3′:3,4]cyclopenta[1,2-d]pyrimidine]. MS (ESI) [M+H]⁺: m/z 733.

### Step G: 8′-(6-chloro-5-cyclopropyl-1H-indazol-4-yl)-2′-(((2R,7aS)-2-fluorotetrahydro-1H-pyrrolizin-7a(5H)-yl)methoxy)spiro[cyclopropane-1,9′-pyrido[4′,3′:3,4]cyclopenta[1,2-d]pyrimidin]-4′-ol

To a solution of 4′-(benzyloxy)-8′-(6-chloro-5-cyclopropyl-1-(tetrahydro-2H-pyran-2-yl)-1H-indazol-4-yl)-2′-(((2R,7aS)-2-fluorotetrahydro-1H-pyrrolizin-7a(5H)-yl)methoxy)spiro[cyclopropane-1,9′-pyrido[4′,3′:3,4]cyclopenta[1,2-d]pyrimidine](150 mg, 0.205 mmol) in DCM (0.2 mL) was added TFA (2.0 mL, 26.0 mmol). The reaction was stirred at 25° C. for 1 h. The reaction was concentrated in vacuo to give 8′-(6-chloro-5-cyclopropyl-1H-indazol-4-yl)-2′-(((2R,7aS)-2-fluorotetrahydro-1H-pyrrolizin-7a(5H)-yl)methoxy)spiro[cyclopropane-1,9′-pyrido[4′,3′:3,4]cyclopenta[1,2-d]pyrimidin]-4′-ol. MS (ESI) [M+H]⁺: m/z 559.

### Step H: tert-butyl (1R,5S)-3-(8′-(6-chloro-5-cyclopropyl-1H-indazol-4-yl)-2′-(((2R,7aS)-2-fluorotetrahydro-1H-pyrrolizin-7a(5H)-yl)methoxy)spiro[cyclopropane-1,9′-pyrido[4′,3′:3,4]cyclopenta[1,2-d]pyrimidin]-4′-yl)-3,8-diazabicyclo[3.2.1]octane-8-carboxylate

To a solution of 8′-(6-chloro-5-cyclopropyl-1H-indazol-4-yl)-2′-(((2R,7aS)-2-fluorotetrahydro-1H-pyrrolizin-7a(5H)-yl)methoxy)spiro[cyclopropane-1,9′-pyrido[4′,3′:3,4]cyclopenta[1,2-d]pyrimidin]-4′-ol (114 mg, 0.204 mmol) in MeCN (2 mL) was added BOP (180 mg, 0.408 mmol) at 25° C., and the reaction mixture was stirred at 25° C. for 0.5 h under N₂ atmosphere. Then to the reaction mixture was added tert-butyl (1R,5S)-3,8-diazabicyclo[3.2.1]octane-8-carboxylate (87 mg, 0.408 mmol) and DIEA (0.214 mL, 1.22 mmol) at 25° C. The resulting mixture was stirred at 50° C. for 16 h. The reaction was concentrated in vacuo to give a crude which was purified by preparative reverse-phase HPLC (25 to 55% MeCN in H₂O w/0.1% TFA modifier) to give tert-butyl (1R,5S)-3-(8′-(6-chloro-5-cyclopropyl-1H-indazol-4-yl)-2′-(((2R,7aS)-2-fluorotetrahydro-1H-pyrrolizin-7a(5H)-yl)methoxy)spiro[cyclopropane-1,9′-pyrido[4′,3′:3,4]cyclopenta[1,2-d]pyrimidin]-4′-yl)-3,8-diazabicyclo[3.2.1]octane-8-carboxylate. MS (ESI) [M+H]⁺: m/z 753.

### Step I: tert-butyl (1R,5S)-3-(8′-(6-chloro-5-cyclopropyl-1H-indazol-4-yl)-2′-(((2R,7aS)-2-fluorotetrahydro-1H-pyrrolizin-7a(5H)-yl)methoxy)spiro[cyclopropane-1,9′-pyrido[4′,3′:3,4]cyclopenta[1,2-d]pyrimidin]-4′-yl)-3,8-diazabicyclo[3.2.1]octane-8-carboxylate

The racemic tert-butyl (1R,5S)-3-(8′-(6-chloro-5-cyclopropyl-1H-indazol-4-yl)-2′-(((2R,7aS)-2-fluorotetrahydro-1H-pyrrolizin-7a(5H)-yl)methoxy)spiro[cyclopropane-1,9′-pyrido[4′,3′:3,4]cyclopenta[1,2-d]pyrimidin]-4′-yl)-3,8-diazabicyclo[3.2.1]octane-8-carboxylate (90 mg, 0.119 mmol) was separated by preparative SFC (Column E, 35% (i-PrOH/ACN=2/1) in n-heptane w/0.1% NH₄OH) to give tert-butyl (1R,5S)-3-(8′-(6-chloro-5-cyclopropyl-1H-indazol-4-yl)-2′-(((2R,7aS)-2-fluorotetrahydro-1H-pyrrolizin-7a(5H)-yl)methoxy)spiro[cyclopropane-1,9′-pyrido[4′,3′:3,4]cyclopenta[1,2-d]pyrimidin]-4′-yl)-3,8-diazabicyclo[3.2.1]octane-8-carboxylate (the second eluting isomer from SFC). MS (ESI) [M+H]⁺: m/z 753.

### Step J: 4′-((1R,5S)-3,8-diazabicyclo[3.2.1]octan-3-yl)-8′-(6-chloro-5-cyclopropyl-1H-indazol-4-yl)-2′-(((2R,7aS)-2-fluorotetrahydro-1H-pyrrolizin-7a(5H)-yl)methoxy)spiro[cyclopropane-1,9′-pyrido[4′,3′:3,4]cyclopenta[1,2-d]pyrimidine] (Ex-115)

To a solution of tert-butyl (1R,5S)-3-(8′-(6-chloro-5-cyclopropyl-1H-indazol-4-yl)-2′-(((2R,7aS)-2-fluorotetrahydro-1H-pyrrolizin-7a(5H)-yl)methoxy)spiro[cyclopropane-1,9′-pyrido[4′,3′:3,4]cyclopenta[1,2-d]pyrimidin]-4′-yl)-3,8-diazabicyclo[3.2.1]octane-8-carboxylate (the second eluting isomer from SFC) (25 mg, 0.033 mmol) in DCM (0.5 mL) was added zinc bromide (74.7 mg, 0.332 mmol) at 25° C., and the reaction mixture was stirred at 25° C. for 30 h under N₂ atmosphere. The reaction was concentrated in vacuo to give a crude residue which was purified by preparative reverse-phase HPLC (0 to 30% water/MeCN w/0.2% formic acid modifier) to give 4′-((1R,5S)-3,8-diazabicyclo[3.2.1]octan-3-yl)-8′-(6-chloro-5-cyclopropyl-1H-indazol-4-yl)-2′-(((2R,7aS)-2-fluorotetrahydro-1H-pyrrolizin-7a(5H)-yl)methoxy)spiro[cyclopropane-1,9′-pyrido[4′,3′:3,4]cyclopenta[1,2-d]pyrimidine](Ex-115). ¹H NMR (400 MHz, CD₃OD) δ 8.61 (d, J=5.4 Hz, 1H), 7.81 (d, J=0.8 Hz, 1H), 7.70 (d, J=5.4 Hz, 1H), 7.39 (d, J=0.8 Hz, 1H), 5.49-5.28 (m, 1H), 4.55-4.41 (m, 2H), 4.40-4.30 (m, 2H), 4.08 (br s, 2H), 3.81 (br t, J=14.2 Hz, 2H), 3.59-3.43 (m, 3H), 3.19 (dt, J=5.7, 9.9 Hz, 1H), 2.53-2.33 (m, 2H), 2.27-2.19 (m, 1H), 2.17-2.09 (m, 2H), 2.08-1.96 (m, 5H), 1.87-1.73 (m, 2H), 1.57-1.44 (m, 2H), 0.95-0.84 (m, 1H), 0.80-0.70 (m, 1H), 0.57-0.47 (m, 1H), 0.39-0.26 (m, 2H). MS (ESI) [M+H]⁺: m/z 653.

The compound in the table below was synthesized using a similar procedure as described in the synthesis of Ex-113 by making the appropriate substitutions for starting material, intermediates, and/or reagents. Appropriate substitutions are available commercially, synthesized as described in the literature, synthesized using methods available to those skilled in the art, or synthesized as described herein.

| | | | | [M + | |
|---|---|---|---|---|---|
| | Starting | | | H]⁺ | Condi- |
| Ex. | Material | Structure | Compound Name | Found | tions |
| Ex- 116 | 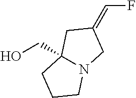 [SMILES: OC[C@@]12CCCN1C/C(=C\F)C2] Int-27 | 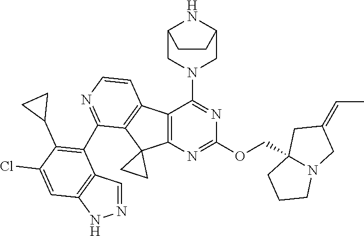 [SMILES: C/C=C1\CN2CCC[C@@]2(COc2nc(N3CC4CCC(C3)N4)c3c(n2)C2(CC2)c2c-3ccnc2-c2c(C3CC3)c(Cl)cc3[nH]ncc23)C1] | 4′-((1R,5S)-3,8-di- azabicyclo[3.2.1]oc- tan-3-yl)-8′-(6- chloro-5-cyclo- propyl-1H- indazol-4-yl)- 2′-(((S,Z)-2- (fluoromethyl- ene)tetrahydro- 1H-pyrrolizin- 7a(5H)-yl)meth- oxy)spiro[cyclo- propane-1,9′-py- rido[4′,3′:3,4]cy- clopenta[1,2-d] | 665 | SFC separa- tion: Column A, 60% EtOH w/CO₂. Second eluting isomer |
| | | | pyrimidine] | | |

### Example 117: 4′-((1R,5S)-3,8-diazabicyclo[3.2.1]octan-3-yl)-8′-(5-chloro-6-fluoro-1H-benzo[f]indazol-4-yl)-2′-(((2R,7aS)-2-fluorotetrahydro-1H-pyrrolizin-7a(5H)-yl)methoxy)spiro[cyclopropane-1,9′-pyrido[4′,3′:3,4]cyclopenta[1,2-d]pyrimidine](Ex-117)

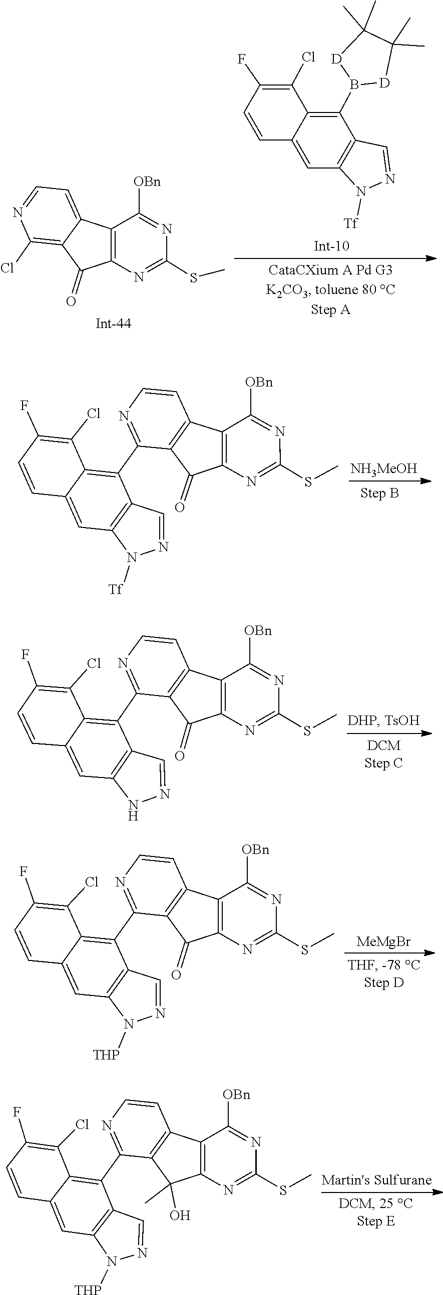

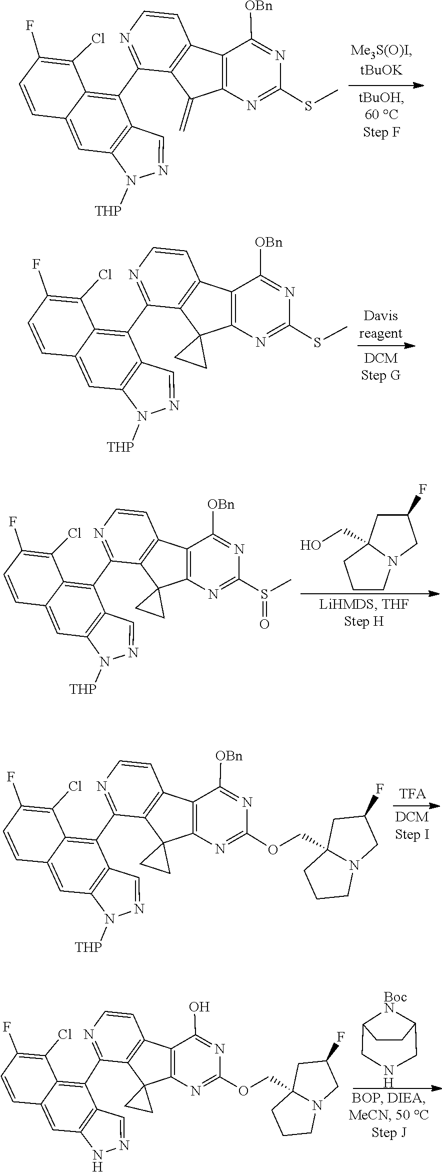

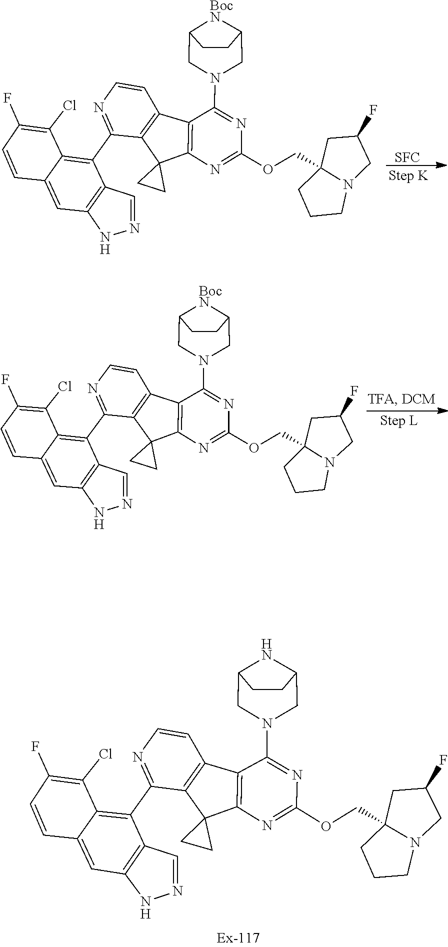

### Step A: 4-(benzyloxy)-8-(5-chloro-6-fluoro-1-((trifluoromethyl)sulfonyl)-1H-benzo[f]indazol-4-yl)-2-(methylthio)-9H-pyrido[4′,3′:3,4]cyclopenta[1,2-d]pyrimidin-9-one

To a solution of 4-(benzyloxy)-8-chloro-2-(methylthio)-9H-pyrido[4′,3′:3,4]cyclopenta[1,2-d]pyrimidin-9-one (Int-44) (1.5 g, 4.06 mmol) in toluene (30 mL) was added 5-chloro-6-fluoro-4-(4,4,5,5-tetramethyl-1,3,2-dioxaborolan-2-yl)-1-((trifluoromethyl)sulfonyl)-1H-benzo[f]indazole (Int-10) (2.48 g, 5.07 mmol), CataCXiumA Pd G3 (1.477 g, 2.028 mmol) and potassium carbonate (10.14 mL, 20.28 mmol, 2M in H₂O) at 25° C., and the reaction mixture was stirred at 80° C. for 2 h under N₂ atmosphere. The reaction mixture was cooled to room temperature and quenched with water (30 mL), and the resulting mixture was extracted with EtOAc (100 mL×3). The combined organic phase was washed with brine, dried over anhydrous Na₂SO₄, filtered and concentrated in vacuo. The residue was purified by preparative TLC (Pet. ether/DCM=1/2) to give 4-(benzyloxy)-8-(5-chloro-6-fluoro-1-((trifluoromethyl)sulfonyl)-1H-benzo[f]indazol-4-yl)-2-(methylthio)-9H-pyrido[4′,3′:3,4]cyclopenta[1,2-d]pyrimidin-9-one. MS (ESI) [M+H]⁺: m/z 686.

### Step B: 4-(benzyloxy)-8-(5-chloro-6-fluoro-1H-benzo[f]indazol-4-yl)-2-(methylthio)-9H-pyrido[4′,3′:3,4]cyclopenta[1,2-d]pyrimidin-9-one

To a solution 4-(benzyloxy)-8-(5-chloro-6-fluoro-1-((trifluoromethyl)sulfonyl)-1H-benzo[f]indazol-4-yl)-2-(methylthio)-9H-pyrido[4′,3′:3,4]cyclopenta[1,2-d]pyrimidin-9-one (1.3 g, 1.9 mmol) in DCM (20 mL) was added ammonia (10 mL, 70.0 mmol, 7M in MeOH), and the reaction mixture was stirred at 25° C. for 16 h. The reaction mixture was concentrated in vacuo to give 4-(benzyloxy)-8-(5-chloro-6-fluoro-1H-benzo[f]indazol-4-yl)-2-(methylthio)-9H-pyrido[4′,3′:3,4]cyclopenta[1,2-d]pyrimidin-9-one. MS (ESI) [M+H]⁺: m/z 554.

### Step C: 4-(benzyloxy)-8-(5-chloro-6-fluoro-1-(tetrahydro-2H-pyran-2-yl)-1H-benzo[f]indazol-4-yl)-2-(methylthio)-9H-pyrido[4′,3′:3,4]cyclopenta[1,2-d]pyrimidin-9-one

To a solution of 4-(benzyloxy)-8-(5-chloro-6-fluoro-1H-benzo[f]indazol-4-yl)-2-(methylthio)-9H-pyrido[4′,3′:3,4]cyclopenta[1,2-d]pyrimidin-9-one (1.05 g, 1.90 mmol) in DCM (10 mL) was added 4-toluenesulfonic acid (0.049 g, 0.28 mmol) and DHP (0.347 mL, 3.79 mmol), and the reaction mixture was stirred at 25° C. for 16 h. The reaction mixture was concentrated in vacuo, and the residue was purified by flash silica gel chromatography (DCM/EtOAc=10/1) to give 4-(benzyloxy)-8-(5-chloro-6-fluoro-1-(tetrahydro-2H-pyran-2-yl)-1H-benzo[f]indazol-4-yl)-2-(methylthio)-9H-pyrido[4′,3′:3,4]cyclopenta[1,2-d]pyrimidin-9-one. MS (ESI) [M+H]⁺: m/z 638.

### Step D: 4-(benzyloxy)-8-(5-chloro-6-fluoro-1-(tetrahydro-2H-pyran-2-yl)-1H-benzo[f]indazol-4-yl)-9-methyl-2-(methylthio)-9H-pyrido[4′,3′:3,4]cyclopenta[1,2-d]pyrimidin-9-ol

To a solution of 4-(benzyloxy)-8-(5-chloro-6-fluoro-1-(tetrahydro-2H-pyran-2-yl)-1H-benzo[f]indazol-4-yl)-2-(methylthio)-9H-pyrido[4′,3′:3,4]cyclopenta[1,2-d]pyrimidin-9-one (620 mg, 0.972 mmol) in THF (10 mL) was added methylmagnesium bromide solution (0.713 mL, 2.14 mmol, 3M in THF) at −78° C., and the reaction mixture was stirred at −78° C. for 40 min under N₂ atmosphere. The reaction mixture was quenched with sat. aq. NH₄Cl solution (5 mL), and the resulting mixture was extracted with EtOAc (30 mL×2). The combined organic phase was washed with brine, dried over anhydrous Na₂SO₄, filtered and concentrated in vacuo to give 4-(benzyloxy)-8-(5-chloro-6-fluoro-1-(tetrahydro-2H-pyran-2-yl)-1H-benzo[f]indazol-4-yl)-9-methyl-2-(methylthio)-9H-pyrido[4′,3′:3,4]cyclopenta[1,2-d]pyrimidin-9-ol. MS (ESI) [M+H]⁺: m/z 654.

### Step E: 4-(benzyloxy)-8-(5-chloro-6-fluoro-1-(tetrahydro-2H-pyran-2-yl)-1H-benzo[f]indazol-4-yl)-9-methylene-2-(methylthio)-9H-pyrido[4′,3′:3,4]cyclopenta[1,2-d]pyrimidine

To a stirred solution of 4-(benzyloxy)-8-(5-chloro-6-fluoro-1-(tetrahydro-2H-pyran-2-yl)-1H-benzo[f]indazol-4-yl)-9-methyl-2-(methylthio)-9H-pyrido[4′,3′:3,4]cyclopenta[1,2-d]pyrimidin-9-ol (636 mg, 0.972 mmol) in DCM (12 mL) was added Martin's sulfurane (981 mg, 1.46 mmol) at 25° C. in a glovebox, and the reaction was stirred at 25° C. for 1 h under N₂ atmosphere. The reaction mixture was concentrated in vacuo, and the residue was purified by flash silica gel chromatography (Pet. ether/THF=3/1) to give 4-(benzyloxy)-8-(5-chloro-6-fluoro-1-(tetrahydro-2H-pyran-2-yl)-1H-benzo[f]indazol-4-yl)-9-methylene-2-(methylthio)-9H-pyrido[4′,3′:3,4]cyclopenta[1,2-d]pyrimidine. MS (ESI) [M+H]⁺: m/z 636.

### Step F: 4′-(benzyloxy)-8′-(5-chloro-6-fluoro-1-(tetrahydro-2H-pyran-2-yl)-1H-benzo[f]indazol-4-yl)-2′-(methylthio)spiro[cyclopropane-1,9′-pyrido[4′,3′:3,4]cyclopenta[1,2-d]pyrimidine]

To a stirred solution of trimethylsulfoxonium iodide (162 mg, 0.736 mmol) in t-BuOH (5 mL) was added potassium tert-butoxide (83 mg, 0.74 mmol) at 25° C., and the reaction mixture was stirred at 60° C. for 0.5 h. Then 4-(benzyloxy)-8-(5-chloro-6-fluoro-1-(tetrahydro-2H-pyran-2-yl)-1H-benzo[f]indazol-4-yl)-9-methylene-2-(methylthio)-9H-pyrido[4′,3′:3,4]cyclopenta[1,2-d]pyrimidine (360 mg, 0.566 mmol)) in t-BuOH (2 mL) and DCM (2 mL) was added to the above mixture, and the mixture was stirred at 60° C. for 1 h under N₂ atmosphere. The reaction mixture was quenched with sat. aq. NH₄Cl (2 mL), and the resulting mixture was extracted with EtOAc (20 mL×3). The combined organic phase was washed with brine, dried over Na₂SO₄, filtered and concentrated in vacuo. The residue was purified by flash silica gel chromatography (Pet. ether/THF=4/1) to give 4′-(benzyloxy)-8′-(5-chloro-6-fluoro-1-(tetrahydro-2H-pyran-2-yl)-1H-benzo[f]indazol-4-yl)-2′-(methylthio)spiro[cyclopropane-1,9′-pyrido[4′,3′:3,4]cyclopenta[1,2-d]pyrimidine]. MS (ESI) [M+H]⁺: m/z 650.

### Step G: 4′-(benzyloxy)-8′-(5-chloro-6-fluoro-1-(tetrahydro-2H-pyran-2-yl)-1H-benzo[f]indazol-4-yl)-2′-(methylsulfinyl)spiro[cyclopropane-1,9′-pyrido[4′,3′:3,4]cyclopenta[1,2-d]pyrimidine]

To a stirred solution of 4′-(benzyloxy)-8′-(5-chloro-6-fluoro-1-(tetrahydro-2H-pyran-2-yl)-1H-benzo[f]indazol-4-yl)-2′-(methylthio)spiro[cyclopropane-1,9′-pyrido[4′,3′:3,4]cyclopenta[1,2-d]pyrimidine](265 mg, 0.408 mmol) in DCM (0.5 mL) was added 3-phenyl-2-(phenylsulfonyl)-1,2-oxaziridine (234 mg, 0.897 mmol) at 25° C., and the reaction mixture was stirred at 25° C. for 0.5 h. The reaction mixture was concentrated in vacuo, and the residue was purified by flash silica gel chromatography (Pet. ether/THF=1/1) to give 4′-(benzyloxy)-8′-(5-chloro-6-fluoro-1-(tetrahydro-2H-pyran-2-yl)-1H-benzo[f]indazol-4-yl)-2′-(methylsulfinyl)spiro[cyclopropane-1,9′-pyrido[4′,3′:3,4]cyclopenta[1,2-d]pyrimidine]. MS (ESI) [M+H]⁺: m/z 666.

### Step H: 4′-(benzyloxy)-8′-(5-chloro-6-fluoro-1-(tetrahydro-2H-pyran-2-yl)-1H-benzo[f]indazol-4-yl)-2′-(((2R,7aS)-2-fluorotetrahydro-1H-pyrrolizin-7a(5H)-yl)methoxy)spiro[cyclopropane-1,9′-pyrido[4′,3′:3,4]cyclopenta[1,2-d]pyrimidine]

To a stirred solution of 4′-(benzyloxy)-8′-(5-chloro-6-fluoro-1-(tetrahydro-2H-pyran-2-yl)-1H-benzo[f]indazol-4-yl)-2′-(methylsulfinyl)spiro[cyclopropane-1,9′-pyrido[4′,3′:3,4]cyclopenta[1,2-d]pyrimidine](80 mg, 0.120 mmol) and ((2R,7aS)-2-fluorotetrahydro-1H-pyrrolizin-7a(5H)-yl)methanol (38.2 mg, 0.240 mmol) in THF (2 mL) was added LiHMDS (0.240 mL, 0.240 mmol, 1M in THF) at 0° C., and the reaction mixture was stirred at 0° C. for 0.5 h. The reaction mixture was quenched with sat. aq. NH₄Cl (2 mL), and the resulting mixture was extracted with EtOAc (20 mL×3). The combined organic phase was washed with brine, dried over Na₂SO₄, filtered and concentrated in vacuo. The residue was purified by preparative TLC (Pet. ether/THF=1/2) to give 4′-(benzyloxy)-8′-(5-chloro-6-fluoro-1-(tetrahydro-2H-pyran-2-yl)-1H-benzo[f]indazol-4-yl)-2′-(((2R,7aS)-2-fluorotetrahydro-1H-pyrrolizin-7a(5H)-yl)methoxy)spiro[cyclopropane-1,9′-pyrido[4′,3′:3,4]cyclopenta[1,2-d]pyrimidine]. MS (ESI) [M+H]⁺: m/z 761.

### Step I: 8′-(5-chloro-6-fluoro-1H-benzo[f]indazol-4-yl)-2′-(((2R,7aS)-2-fluorotetrahydro-1H-pyrrolizin-7a(5H)-yl)methoxy)spiro[cyclopropane-1,9′-pyrido[4′,3′:3,4]cyclopenta[1,2-d]pyrimidin]-4′-ol

To a solution of 4′-(benzyloxy)-8′-(5-chloro-6-fluoro-1-(tetrahydro-2H-pyran-2-yl)-1H-benzo[f]indazol-4-yl)-2′-(((2R,7aS)-2-fluorotetrahydro-1H-pyrrolizin-7a(5H)-yl)methoxy)spiro[cyclopropane-1,9′-pyrido[4′,3′:3,4]cyclopenta[1,2-d]pyrimidine](90 mg, 0.12 mmol) in DCM (1 mL) was added TFA (0.5 mL, 6.49 mmol)) at 25° C., and the reaction mixture was stirred at 25° C. for 16 h. The reaction mixture was concentrated in vacuo to give 8′-(5-chloro-6-fluoro-1H-benzo[f]indazol-4-yl)-2′-(((2R,7aS)-2-fluorotetrahydro-1H-pyrrolizin-7a(5H)-yl)methoxy)spiro[cyclopropane-1,9′-pyrido[4′,3′:3,4]cyclopenta[1,2-d]pyrimidin]-4′-ol. MS (ESI) [M+H]⁺: m/z 587.

### Step J: tert-butyl (1R,5S)-3-(8′-(5-chloro-6-fluoro-1H-benzo[f]indazol-4-yl)-2′-(((2R,7aS)-2-fluorotetrahydro-1H-pyrrolizin-7a(5H)-yl)methoxy)spiro[cyclopropane-1,9′-pyrido[4′,3′:3,4]cyclopenta[1,2-d]pyrimidin]-4′-yl)-3,8-diazabicyclo[3.2.1]octane-8-carboxylate

To a solution of 8′-(5-chloro-6-fluoro-1H-benzo[f]indazol-4-yl)-2′-(((2R,7aS)-2-fluorotetrahydro-1H-pyrrolizin-7a(5H)-yl)methoxy)spiro[cyclopropane-1,9′-pyrido[4′,3′:3,4]cyclopenta[1,2-d]pyrimidin]-4′-ol (69.4 mg, 0.103 mmol) in MeCN (2 mL) was added DIEA (0.108 mL, 0.620 mmol) and BOP (91 mg, 0.21 mmol), and the mixture was stirred at 25° C. for 0.5 h. Then tert-butyl (1R,5S)-3,8-diazabicyclo[3.2.1]octane-8-carboxylate (43.9 mg, 0.207 mmol) and DIEA (0.108 mL, 0.620 mmol) was added the reaction mixture, and the mixture was stirred at 50° C. for 1 h. The reaction mixture was directly purified by preparative TLC (DCM/MeOH=10/1) to give tert-butyl (1R,5S)-3-(8′-(5-chloro-6-fluoro-1H-benzo[f]indazol-4-yl)-2′-(((2R,7aS)-2-fluorotetrahydro-1H-pyrrolizin-7a(5H)-yl)methoxy)spiro[cyclopropane-1,9′-pyrido[4′,3′:3,4]cyclopenta[1,2-d]pyrimidin]-4′-yl)-3,8-diazabicyclo[3.2.1]octane-8-carboxylate. MS (ESI) [M+H]⁺: m/z 781.

### Step K: tert-butyl (1R,5S)-3-(8′-(5-chloro-6-fluoro-1H-benzo[f]indazol-4-yl)-2′-(((2R,7aS)-2-fluorotetrahydro-1H-pyrrolizin-7a(5H)-yl)methoxy)spiro[cyclopropane-1,9′-pyrido[4′,3′:3,4]cyclopenta[1,2-d]pyrimidin]-4′-yl)-3,8-diazabicyclo[3.2.1]octane-8-carboxylate

The racemic tert-butyl (1R,5S)-3-(8′-(5-chloro-6-fluoro-1H-benzo[f]indazol-4-yl)-2′-(((2R,7aS)-2-fluorotetrahydro-1H-pyrrolizin-7a(5H)-yl)methoxy)spiro[cyclopropane-1,9′-pyrido[4′,3′:3,4]cyclopenta[1,2-d]pyrimidin]-4′-yl)-3,8-diazabicyclo[3.2.1]octane-8-carboxylate (85 mg, 0.11 mmol) was separated by preparative SFC (Column D; 60% EtOH w/NH₄OH modifier) to afford tert-butyl (1R,5S)-3-(8′-(5-chloro-6-fluoro-1H-benzo[f]indazol-4-yl)-2′-(((2R,7aS)-2-fluorotetrahydro-1H-pyrrolizin-7a(5H)-yl)methoxy)spiro[cyclopropane-1,9′-pyrido[4′,3′:3,4]cyclopenta[1,2-d]pyrimidin]-4′-yl)-3,8-diazabicyclo[3.2.1]octane-8-carboxylate (the second eluting isomer). MS (ESI) [M+H]⁺: m/z 781.

### Step L: 4′-((1R,5S)-3,8-diazabicyclo[3.2.1]octan-3-yl)-8′-(5-chloro-6-fluoro-1H-benzo[f]indazol-4-yl)-2′-(((2R,7aS)-2-fluorotetrahydro-1H-pyrrolizin-7a(5H)-yl)methoxy)spiro[cyclopropane-1,9′-pyrido[4′,3′:3,4]cyclopenta[1,2-d]pyrimidine] (Ex-117)

To a solution of tert-butyl (1R,5S)-3-(8′-(5-chloro-6-fluoro-1H-benzo[f]indazol-4-yl)-2′-(((2R,7aS)-2-fluorotetrahydro-1H-pyrrolizin-7a(5H)-yl)methoxy)spiro[cyclopropane-1,9′-pyrido[4′,3′:3,4]cyclopenta[1,2-d]pyrimidin]-4′-yl)-3,8-diazabicyclo[3.2.1]octane-8-carboxylate (20 mg, 0.026 mmol, second eluting isomer) in DCM (0.5 mL) was added TFA (0.25 mL, 3.24 mmol) at 25° C., and the reaction mixture was stirred at 25° C. for 0.5 h under N₂ atmosphere. The mixture was concentrated in vacuo, and the mixture was filtered, and the filtrate was purified by preparative reverse-phase HPLC (10 to 30% MeCN in H₂O w/0.2% formic acid modifier) to give 4′-((1R,5S)-3,8-diazabicyclo[3.2.1]octan-3-yl)-8′-(5-chloro-6-fluoro-1H-benzo[f]indazol-4-yl)-2′-(((2R,7aS)-2-fluorotetrahydro-1H-pyrrolizin-7a(5H)-yl)methoxy)spiro[cyclopropane-1,9′-pyrido[4′,3′:3,4]cyclopenta[1,2-d]pyrimidine](Ex-117). ¹H NMR (400 MHz, CD₃OD) δ 8.68-8.60 (m, 1H), 8.41-8.34 (m, 1H), 8.24-8.13 (m, 1H), 7.80-7.72 (m, 1H), 7.60 (s, 1H), 7.48 (t, J=8.8 Hz, 1H), 5.49-5.30 (m, 1H), 4.62 (br s, 1H), 4.44-4.26 (m, 3H), 4.16-4.05 (m, 2H), 3.95-3.87 (m, 1H), 3.77 (br d, J=13.7 Hz, 1H), 3.57-3.40 (m, 3H), 3.19 (dt, J=5.9, 9.9 Hz, 1H), 2.51-2.38 (m, 1H), 2.37-2.31 (m, 1H), 2.27-2.11 (m, 4H), 2.08-1.90 (m, 4H), 1.60-1.51 (m, 1H), 1.48-1.39 (m, 1H), 1.18-1.04 (m, 1H), 0.65-0.54 (m, 1H). MS (ESI) [M+H]⁺: m/z 681.

### Example 118: 4-((1R,5S)-3,8-diazabicyclo[3.2.1]octan-3-yl)-8-(6-chloro-5-cyclopropyl-1H-indazol-4-yl)-2-((4-(trifluoromethyl)tetrahydro-2H-pyran-4-yl)methoxy)pyrido[4′,3′:4,5]thieno[2,3-d]pyrimidine (Ex-118)

Example 118 in the table below was synthesized using a similar procedure as described in the synthesis of Ex-20 by making the appropriate substitutions for starting material, intermediates, and/or reagents. Appropriate substitutions are available commercially, synthesized as described in the literature, synthesized using methods available to those skilled in the art, or synthesized as described herein.

| | | | | [M + |
|---|---|---|---|---|
| | | | | H]⁺ |
| Ex. | Starting Material | Structure | Compound Name | Found |
| Ex-118 | 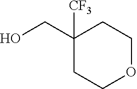 [SMILES: OCC1(C(F)(F)F)CCOCC1] (4- (trifluoromethyl)tetra- hydro-2H-pyran-4- yl)methanol | 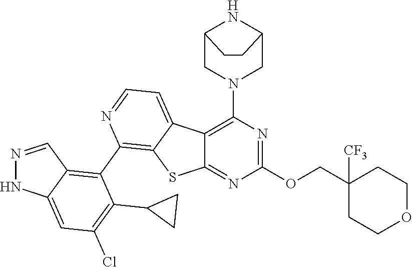 [SMILES: FC(F)(F)C1(COc2nc(N3CC4CCC(C3)N4)c3c(n2)sc2c(-c4c(C5CC5)c(Cl)cc5[nH]ncc45)nccc23)CCOCC1] | 4-((1R,5S)-3,8- diazabicyclo [3.2.1]octan-3- yl)-8-(6-chloro-5- cyclopropyl-1H- indazol-4-yl)-2- ((4-(trifluoro- methyl)tetra- hydro-2H-pyran- 4-yl)meth- oxy)pyrido [4′,3′:4,5]thi- eno[2,3-d]py- rimidine | 670 |

### Assays

### Procedure for SOS-Catalyzed Nucleotide Exchange Assay for KRAS-WT, G12C/D/V, G13D, HRAS, and NRAS (Procedure A)

Recombinant KRAS-G12C protein used in this assay has an additional triple mutation (C51S/C80L/C118S). Whereas KRAS-WT, G12D/V, G13D, HRAS and NRAS are in context of their WT protein sequence background. Specifically, the SOS-catalyzed nucleotide exchange assay utilizes a preformed TR-FRET complex containing a specific biotinylated RAS protein (KRAS-WT, G12C/V/D, G13D, HRAS, NRAS; described above) with Bodipy-GDP, and Terbium-streptavidin. Compounds are preincubated with this complex for 60 minutes. Subsequently, recombinant human SOS protein and unlabeled GTP are added to initiate the exchange reaction. Small molecule inhibitors stabilize the Bodipy-GDP complex whereas the untreated protein rapidly exchanges Bodipy-GDP for unlabeled GTP resulting in reduced TR-FRET signal.

To assemble the preformed TR-FRET complexes, each biotinylated RAS protein is diluted to 2 μM in an EDTA Buffer (20 mM HEPES pH 7.5, 50 mM sodium chloride, 10 mM EDTA, and 0.01% Tween) and incubated at room temperature for one hour. This mixture is then further diluted to 90 nM in an Assay Buffer (20 mM HEPES pH 7.5, 150 mM sodium chloride, 10 mM magnesium chloride, and 0.005% Tween) containing 15 nM of Terbium-Streptavidin (Invitrogen, catalog #PV3577) and 900 nM of Bodipy-GDP (Invitrogen, catalog #G22360) and incubated at room temperature for six hours. It should be noted that this preformed TR-FRET complex for each of the RAS protein were made ahead of time, aliquoted and stored at −80° C. until the day of the experiment.

Each test compound (10 mM stock in DMSO) is diluted in DMSO to make a final-10-point, 3-fold dilution and is acoustically dispensed into a 384-well assay plate (Corning, catalog #3820) using an Echo 550 (Labcyte). Each well of the assay plate receives 3 μL of a specific 3×RAS preformed TR-FRET complex and 3 μL of Assay Buffer and is incubated at room temperature for 60 minutes (preincubation time). Each well then receives 3 μL of 3× recombinant human SOS protein and GTP (Sigma, G8877) in Assay Buffer and is incubated at room temperature for 30 minutes (G13D), 60 minutes (KRAS WT/G12C/D, H/NRAS) or 90 minutes for G12V. The final reaction in each well of 9 μL consists of 3 mM GTP, specific Ras and SOS proteins in the following concentrations: KRAS-G12C/SOS=3 nM/40 nM, KRAS-WT and G12D/SOS=1 nM/40 nM, KRAS-G12V/SOS=1.25 nM/160 nM, KRAS-G13D/SOS=1.25 nM/0 nM, HRAS and NRAS=1.25 nM/40 nM.

The time-resolved fluorescence resonance energy transfer (TR-FRET) signal is measured on an Envision (PerkinElmer) plate reader: Excitation filter=340 nm; emission1=495 nm; emission2=520 nm; dichroic mirror=D400/D505; delay time=100 ms. The signal of each well is determined as the ratio of the emission at 520 nm to that at 495 nm. Percent effect of each well is determined after normalization to control wells containing DMSO (no effect) or a saturating concentration of inhibitor (max effect). The apparent effect as a function of compound concentration is fit to a four-parameter logistic equation.

### Procedure for SOS-Catalyzed Nucleotide Exchange Assay for KRAS-G12C/D/V/WT (Procedure B)

Recombinant KRAS G12C (amino acids 1-169, SEQ ID NO:9), KRAS G12D (amino acids 1-169, SEQ ID NO:10), KRAS G12V (amino acids 1-169, SEQ ID NO:11), KRAS WT (amino acids 1-169, SEQ ID NO:12) and SOS1 (amino acids 564-1049, SEQ ID NO:13) proteins were expressed in E. coli and purified by affinity chromatography. To prepare each BODIPY™ FL GDP-bound KRAS protein, 50 μM KRAS proteins were incubated with 0.5 mM BODIPY™ FL GDP (Invitrogen, G22360) in a loading buffer (20 mM Tris-HCl pH 7.5, 50 mM NaCl, 1 mM DTT and 2.5 mM EDTA) for 1 hour on ice. After the incubation, MgCl₂ was added to a final concentration of 10 mM, followed by an incubation at room temperature for 30 minutes. The mixtures were allowed to pass through a NAP-5 column to remove free nucleotides and purified BODIPY™ FL GDP-bound KRAS G12C, G12D, G12V and WT proteins were used for compound evaluation.

The inhibitory activity of compounds on recombinant KRAS is measured by the displacement of the bound BODIPY™ GDP. Specifically, 2.5 nM of each BODIPY™ FL GDP-bound KRAS complex was incubated with various concentrations of compound in a reaction buffer (20 mM Tris-HCl pH 7.5, 100 mM NaCl, 1 mM MgCl₂, 2 mM DTT, 0.1% Tween 20) at 25° C. for 1 hour. After the incubation, recombinant SOS1 and GMPPNP (Jena Bioscience GmbH, NU-401) were added and incubated at room temperature for 30 minutes to proceed SOS1-dependent GDP-GTP exchange reaction on KRAS. Displacement of BODIPY™ FL GDP by Guanosine-5′-[(β,γ)-imido]triphosphate, Tetralithium salt (GMPPNP) was measured by calculating the ratio of fluorescence intensities of BODIPY™ FL before and after the exchange reaction. Percent Inhibition was calculated by setting the fluorescence ratio from the reaction without test compound (DMSO control) and the fluorescence ratio from the reaction without SOS1 and GMPPNP as 0% and 100% inhibition, respectively. Dose response curves were analyzed using a 4-parameter logistic model to calculate IC₅₀ values.

### Procedure for Cellular Phospho-ERK Assay in KRAS Wild Type and Mutant (G12D, G12V) Cell Lines

PANC08.13 cells (ATCC® CRL-2551™), containing homozygous KRAS-G12D activating mutation, were cultured in growth medium made of RPMI1640-GlutaMAX™-I (ThermoFisher Scientific 61870) containing 15% heat inactivated fetal bovine serum (ThermoFisher Scientific 10091148)). AsPC-1 cells (ATCC® CRL-1682™), containing homozygous KRAS-G12D activating mutation, were cultured in T150 flask in growth medium (RPMI medium 1640-GlutaMAX™-I (ThermoFisher Scientific 61870) containing 10% fetal bovine serum (ThermoFisher Scientific 10091148)).

Cells for the assay were harvested in growth medium after TrypLE (ThermoFisher scientific 12604021) digestion and were seeded in a 384-well collagen coated cell culture plate (Corning 356702) at a density of 10,000-15,000 cells/20 μL/well, and incubated at 37° C., 5% CO₂ overnight. The compound (with 10 mM stock concentration) dose-response titrations were prepared [30 μM final ERK detection assay concentration and 1:3 dilutions, 10-point dose response] and appropriate amounts (270 nL) of test compounds were dispensed in a 384-well intermediate plate using an Echo 550 liquid handler. 30 μL/well of RPMI medium 1640-GlutaMAX™-I was added to the intermediate plate and the contents of the intermediate plate (10 μL/well) were then transferred to the 384-well collagen coated cell culture plate, which was incubated at 37° C., 5% CO₂ for 2 hours. After removal of medium from the collagen coated cell culture plate, cells were lysed in lysis buffer from Alpha SureFire® Ultra™ Multiplex p-ERK and total ERK assay kit (PerkinElmer MPSU-PTERK) containing Halt™ Protease and Phosphatase inhibitor cocktail (ThermoFisher Scientific 78446) at room temperature with constant shaking at 300 rpm for 30 minutes. The cell lysates were then transferred to an OptiPlate-384 plate (PerkinElmer 6005620), and the phosphorylation of ERK (p-ERK) and total ERK levels were detected by Alpha SureFire® Ultra™ Multiplex p-EEK kit and total ERK assay kit (PerkinElmer MPSU-PTERK) following the manufacturer's protocol. Assay plates were read on a EnVision Multimode Plate Reader (PerkinElmer), and the ratio of p-ERK vs. total ERK in each well was used as the final readout. Dose response curves were analyzed using a 4-parameter logistic model to calculate IC₅₀ values using Spotfire software. The results of this assay are presented in the table below.

| | KRAS | KRAS | | KRAS | KRAS | | | | PANCO |
|---|---|---|---|---|---|---|---|---|---|
| | G12D | G12V | KRAS | G12C | G13D | | NRAS | AsPC1 | 813 |
| | GNE | GNE | WT GNE | GNE | GNE | HRAS | GNE | pERK | pERK |
| | IC50 | IC50 | IC50 | IC50 | IC50 | GNE IC50 | IC50 | IC50 | IC50 |
| Ex # | (nM) | (nM) | (nM) | (nM) | (nM) | (nM) | (nM) | (nM) | (nM) |
| Ex-1 | 14.28 | 3556 | 6503 | 651.7 | 1643 | >11060 | >11060 | 455.5 | 139.4 |
| Ex-2 | 43.63 | >11110 | >11110 | >11060 | 2363 | >11060 | >11060 | 15090 | 1627 |
| Ex-3 | 97.55 | 5135 | 9310 | 918.7 | 3323 | >11060 | >11060 | 964.7 | 244.4 |
| Ex-4 | 118.8 | >11110 | >11110 | >11060 | 4016 | >11060 | >11060 | — | 3752 |
| Ex-5 | 18.05 | >11110 | >11110 | >11060 | 349.2 | >11060 | >11060 | — | 1185 |
| Ex-6 | 905.6 | >11110 | >11110 | >11060 | >11060 | >11060 | >11060 | — | 9853 |
| Ex-7 | 51.01 | >11110 | >11110 | >11060 | 1448 | >11060 | >11060 | — | 571.7 |
| Ex-8 | 1.809 | 62.65 | 48.08 | 25.23 | 17.81 | >11060 | >11060 | 137.7 | 63.84 |
| Ex-9 | 40.69 | — | — | 1550 | 450.8 | >11060 | >11060 | 490.4 | 315.5 |
| Ex-10 | 7.112 | 6884 | 4934 | 647.2 | 603.6 | >11060 | >11060 | 397.5 | 191.5 |
| Ex-11 | 697.8 | >11110 | >11110 | — | — | — | — | — | 2479 |
| Ex-12 | 6.41 | 2073 | 5805 | 589 | 722.6 | >11060 | >11060 | 199.7 | 88.9 |
| Ex-13 | 13.71 | 8345 | 9555 | 665.3 | 1826 | >11060 | >11060 | 582.5 | 220.5 |
| Ex-14 | 110.2 | >5000 | >5000 | >5000 | — | — | — | — | — |
| Ex-15 | 4993 | >11110 | >11110 | >11060 | >11060 | >11060 | >11060 | — | >10000 |
| Ex-16 | 282.6 | >11110 | >11110 | — | — | — | — | — | 9745 |
| Ex-17 | 23.97 | >11110 | >11110 | >11060 | >11060 | >11060 | >11060 | — | 1636 |
| Ex-18 | 123.5 | >11110 | >11110 | — | — | — | — | — | 665.7 |
| Ex-19 | 23.43 | >11110 | 9937 | 754.5 | 1048 | >11060 | >11060 | 2200 | 249.7 |
| Ex-20 | 8.052 | 1242 | 1155 | 412.4 | 286.6 | >11060 | >11060 | 295.6 | — |
| Ex-21 | 7.894 | 4999 | 2804 | 456.5 | 485.5 | >11060 | >11060 | 612.1 | — |
| Ex-22 | 2732 | >11110 | >11110 | >11060 | 9112 | >11060 | >11060 | 10050 | — |
| Ex-23 | 1970 | >11110 | >11110 | >11060 | >11060 | >11060 | >11060 | 8114 | — |
| Ex-24 | 5227 | >11110 | >11110 | >11060 | >11060 | >11060 | >11060 | 18420 | — |
| Ex-25 | 1025 | >11110 | >11110 | >11060 | >11060 | >11060 | >11060 | — | 2869 |
| Ex-26 | 3195 | >11110 | >11110 | — | — | — | — | >30000 | — |
| Ex-27 | 3588 | >11110 | >11110 | — | — | — | — | 5728 | — |
| Ex-28 | 3339 | >11110 | >11110 | — | — | — | — | >30000 | — |
| Ex-29 | 2483 | >11110 | >11110 | — | — | — | — | >30000 | — |
| Ex-30 | 2603 | >11110 | >11110 | — | — | — | — | >30000 | — |
| Ex-31 | 7791 | >11110 | >11110 | — | — | — | — | >30000 | — |
| Ex-32 | 1021 | >11110 | >11110 | >11060 | >11060 | >11060 | >11060 | >30000 | — |
| Ex-33 | 7972 | >11110 | >11110 | >11060 | >11060 | >11060 | >11060 | 20190 | — |
| Ex-34 | 6697 | >11110 | >11110 | >11060 | >11060 | >11060 | >11060 | >30000 | — |
| Ex-35 | 1427 | >11110 | >11110 | >11060 | >11060 | >11060 | >11060 | >30000 | — |
| Ex-36 | 4119 | >11110 | >11110 | >11060 | >11060 | >11060 | >11060 | 28410 | — |
| Ex-37 | 1276 | >11110 | >11110 | >11060 | >11060 | >11060 | >11060 | >30000 | — |
| Ex-38 | 3791 | >11110 | >11110 | >11060 | 3546 | >11060 | >11060 | >30000 | — |
| Ex-39 | 2419 | >11110 | >11110 | >11060 | >11060 | >11060 | >11060 | 12820 | — |
| Ex-40 | 5884 | >11110 | >11110 | — | — | — | — | >30000 | — |
| Ex-41 | 4636 | — | — | — | — | — | — | >30000 | — |
| Ex-42 | 3104 | — | — | — | — | — | — | 6518 | — |
| Ex-43 | 2180 | — | — | — | — | — | — | 6826 | — |
| Ex-44 | 2639 | — | — | — | — | — | — | 4673 | — |
| Ex-45 | 3007 | — | — | — | — | — | — | 4866 | — |
| Ex-46 | 2812 | — | — | — | — | — | — | 12600 | — |
| Ex-47 | 3130 | — | — | — | — | — | — | 11300 | — |
| Ex-48 | 3349 | — | — | — | — | — | — | 14880 | — |
| Ex-49 | 1462 | — | — | — | — | — | — | 5977 | — |
| Ex-50 | 2377 | — | — | — | — | — | — | >30000 | — |
| Ex-51 | 2150 | >11110 | >11110 | — | — | — | — | — | 6808 |
| Ex-52 | 724.1 | >11110 | >11110 | — | — | — | — | — | 2769 |
| Ex-53 | 583.3 | >11110 | >11110 | >11060 | 4879 | >11060 | >11060 | — | 3972 |
| Ex-54 | 1.916 | 62.74 | 80.71 | 22.75 | 14.83 | >11060 | >11060 | 31.38 | — |
| Ex-55 | 1.312 | 105.4 | 148.3 | 73.87 | 42.45 | >11060 | >11060 | 25.02 | — |
| Ex-56 | 4.449 | 283.5 | 224.8 | — | — | — | — | 31.75 | — |
| Ex-57 | 23.59 | 2091 | 2890 | — | — | — | — | 299.3 | — |
| Ex-58 | 9.305 | 2555 | 1828 | — | — | — | — | 447 | — |
| Ex-59 | 9.251 | 1368 | 1651 | 181.9 | 264.7 | >11060 | >11060 | 216.6 | — |
| Ex-60 | 2688 | >11110 | >11110 | — | — | — | — | — | 10000 |
| Ex-61 | 859.5 | >11110 | >11110 | — | — | — | — | — | 7346 |
| Ex-62 | 645.2 | >11110 | >11110 | 8491 | 1539 | >11060 | >11060 | — | >10000 |
| Ex-63 | 73.73 | >11110 | >11110 | >11060 | >11060 | >11060 | >11060 | — | >10000 |
| Ex-64 | 9416 | >11110 | >11110 | — | — | — | — | — | >10000 |
| Ex-65 | 128 | >11110 | 8792 | 1965 | 2738 | >11060 | >11060 | 1699 | — |
| Ex-66 | 201.8 | >11110 | 6332 | 1694 | 1504 | >11060 | >11060 | — | 1258 |
| Ex-67 | 13.14 | 672.2 | 856.1 | 265.1 | 323.4 | >11060 | >11060 | 699.3 | — |
| Ex-68 | 267.4 | 1361 | 6598 | 1854 | 3051 | >11060 | >11060 | 11140 | — |
| Ex-69 | 35.62 | >11110 | 4914 | 1736 | 1212 | >11060 | >11060 | 1538 | — |
| Ex-70 | 836.1 | 9750 | 7501 | 947.9 | 2262 | >11060 | >11060 | 5111 | — |
| Ex-71 | 34.79 | 5158 | 8086 | 1096 | 2509 | >11060 | >11060 | 885 | — |
| Ex-72 | 5297 | >11110 | >11110 | >11060 | 2623 | >11060 | >11060 | >30000 | — |
| Ex-73 | 1392 | >11110 | >11110 | >11060 | >11060 | >11060 | >11060 | 4795 | — |
| Ex-74 | 1302 | >11110 | >11110 | >11060 | 2877 | >11060 | >11060 | 12150 | — |
| Ex-75 | 38.14 | >11110 | 3679 | — | — | — | — | 629.1 | — |
| Ex-76 | 26.26 | 6354 | 8268 | 1041 | 1815 | >11060 | >11060 | 396.7 | — |
| Ex-77 | 17.08 | 2316 | 4842 | 334.2 | 373.9 | >11060 | >11060 | 726.3 | — |
| Ex-78 | 728.2 | >11110 | >11110 | >11060 | >11060 | >11060 | >11060 | — | 2651 |
| Ex-79 | 12.33 | 4495 | 4810 | — | — | — | — | 612.2 | — |
| Ex-80 | 3.571 | 734 | 706.3 | — | — | — | — | 410.9 | — |
| Ex-81 | 574.6 | >11110 | >11110 | — | — | — | — | 6804 | — |
| Ex-82 | 1.928 | 16.17 | 12.52 | — | — | — | — | 6.286 | — |
| Ex-83 | 1.433 | 703.6 | 429.9 | — | — | — | — | 42.46 | — |
| Ex-84 | 110.9 | >11110 | >11110 | — | — | — | — | 2942 | — |
| Ex-85 | 2.059 | 28.17 | 32.1 | — | — | — | — | 8.524 | — |
| Ex-86 | 3.31 | 1239 | 542.4 | — | — | — | — | 54.65 | — |
| Ex-87 | 3.585 | 412 | 467.1 | — | — | — | — | 86.34 | — |
| Ex-88 | 100.2 | 1776 | 743 | — | — | — | — | 367.9 | — |
| Ex-89 | 1.668 | 48.82 | 32.75 | — | — | — | — | 8.181 | — |
| Ex-90 | 187.3 | >11110 | >11110 | — | — | — | — | 3306 | — |
| Ex-91 | 50.6 | >11060 | >11060 | — | — | — | — | 5029 | — |
| Ex-92 | 1.988 | 88.17 | 57.35 | — | — | — | — | 18.11 | — |
| Ex-93 | 1.161 | 94.31 | 72.87 | — | — | — | — | 18.01 | — |
| Ex-94 | 2.99 | 1952 | 846.9 | — | — | — | — | 85.87 | — |
| Ex-95 | 5.287 | 6762 | 2801 | — | — | — | — | 180.1 | — |
| Ex-96 | 1.621 | 248.2 | 258.8 | — | — | — | — | 22.81 | — |
| Ex-97 | 3.003 | 3135 | 1287 | — | — | — | — | 209.9 | — |
| Ex-98 | 6.923 | >11110 | 6362 | — | — | — | — | 362.2 | — |
| Ex-99 | 1.533 | 2739 | 1078 | — | — | — | — | 59.89 | — |
| Ex-100 | 8.521 | >11110 | >11110 | — | — | — | — | 753 | — |
| Ex-101 | 33.11 | >11060 | >11060 | — | — | — | — | 1207 | — |
| Ex-102 | 12.27 | >11060 | >11060 | — | — | — | — | 2770 | — |
| Ex-103 | 1.995 | 9285 | 2360 | — | — | — | — | 295.6 | — |
| Ex-104 | 1.941 | 631 | 398.8 | — | — | — | — | 48.44 | — |
| Ex-105 | 1.603 | 474.9 | 279.3 | — | — | — | — | 66.97 | — |
| Ex-106 | 2.026 | 1697 | 877.3 | — | — | — | — | 73.98 | — |
| Ex-107 | 3.227 | 1033 | 800.2 | — | — | — | — | 51.79 | — |
| Ex-108 | 1.623 | 371.7 | 215.3 | — | — | — | — | 16.16 | — |
| Ex-109 | 25.85 | >11060 | >11060 | — | — | — | — | 1009 | — |
| Ex-110 | 2053 | >11110 | >11110 | — | — | — | — | 8283 | — |
| Ex-111 | 5.426 | 4396 | 1721 | — | — | — | — | 171.6 | — |
| Ex-112 | 6772 | >11060 | >11060 | — | — | — | — | 14730 | — |
| Ex-113 | 492.5 | 1848 | 1109 | — | — | — | — | 2382 | — |
| Ex-114 | 1.809 | 37.7 | 36.2 | — | — | — | — | 96.98 | — |
| Ex-115 | 8.998 | 2121 | 2048 | — | — | — | — | 522.9 | — |
| Ex-116 | 3.377 | 804.4 | 772 | — | — | — | — | 373.3 | — |
| Ex-117 | 2.404 | 859.2 | 369.1 | — | — | — | — | 188.6 | — |
| Ex-118 | 3201 | >11110 | 6273 | — | — | — | — | 4885 | — |

For Example No. 14, nucleotide exchange assays for KRAS G12D, WT, G12C, and G12V were performed according to Procedure B. All the other compounds were tested according to Procedure A.

SEQUENCES (Procedure A)
Recombinant Human KRAS G12C
SEQ ID NO: 1
GLNDIFEAQKIEWHETEYKLVVVGACGVGKSALTIQLIQNHFVDEYDPTI
EDSYRKQVVIDGETSLLDILDTAGQEEYSAMRDQYMRTGEGFLLVFAINN
TKSFEDIHHYREQIKRVKDSEDVPMVLVGNKSDLPSRTVDTKQAQDLARS
YGIPFIETSAKTRQGVDDAFYTLVREIRKHKEK
Recombinant Human KRAS G12D
SEQ ID NO: 2
GLNDIFEAQKIEWHETEYKLVVVGADGVGKSALTIQLIQNHFVDEYDPTI
EDSYRKQVVIDGETCLLDILDTAGQEEYSAMRDQYMRTGEGFLCVFAINN
TKSFEDIHHYREQIKRVKDSEDVPMVLVGNKCDLPSRTVDTKQAQDLARS
YGIPFIETSAKTRQGVDDAFYTLVREIRKHKEK
Recombinant Human KRAS G12V
SEQ ID NO: 3
GLNDIFEAQKIEWHETEYKLVVVGAVGVGKSALTIQLIQNHFVDEYDPTI
EDSYRKQVVIDGETCLLDILDTAGQEEYSAMRDQYMRTGEGFLCVFAINN
TKSFEDIHHYREQIKRVKDSEDVPMVLVGNKCDLPSRTVDTKQAQDLARS
YGIPFIETSAKTRQGVDDAFYTLVREIRKHKEK
Recombinant Human KRAS G13D
SEQ ID NO: 4
GLNDIFEAQKIEWHETEYKLVVVGAGDVGKSALTIQLIQNHFVDEYDPTI
EDSYRKQVVIDGETCLLDILDTAGQEEYSAMRDQYMRTGEGFLCVFAINN
TKSFEDIHHYREQIKRVKDSEDVPMVLVGNKCDLPSRTVDTKQAQDLARS
YGIPFIETSAKTRQGVDDAFYTLVREIRKHKEK
Recombinant Human HRAS
SEQ ID NO: 5
GGGGSHMTEYKLVVVGAGGVGKSALTIQLIQNHFVDEYDPTIEDSYRKQV
VIDGETCLLDILDTAGQEEYSAMRDQYMRTGEGFLCVFAINNTKSFEDIH
QYREQIKRVKDSDDVPMVLVGNKCDLAARTVESRQAQDLARSYGIPYIET
SAKTRQGVEDAFYTLVREIRQH
Recombinant Human NRAS
SEQ ID NO: 6
GGGGMTEYKLVVVGAGGVGKSALTIQLIQNHFVDEYDPTIEDSYRKQVVI
DGETCLLDILDTAGQEEYSAMRDQYMRTGEGFLCVFAINNSKSFADINLY
REQIKRVKDSDDVPMVLVGNKCDLPTRTVDTKQAHELAKSYGIPFIETSA
KTRQGVEDAFYTLVREIRQYRMKKLN
Recombinant Human SOS Protein
SEQ ID NO: 7
MGSSHHHHHHSGENLYFQGSSGLNDIFEAQKIEWHESSEEQMRLPSADVY
RFAEPDSEENIIFEENMQPKAGIPIIKAGTVIKLIERLTYHMYADPNFVR
TFLTTYRSFCKPQELLSLIIERFEIPEPEPTEADRIAIENGDQPLSAELK
RFRKEYIQPVQLRVLNVCRHWVEHHFYDFERDAYLLQRMEEFIGTVRGKA
MKKWVESITKIIQRKKIARDNGPGHNITFQSSPPTVEWHISRPGHIETFD
LLTLHPIEIARQLTLLESDLYRAVQPSELVGSVWTKEDKEINSPNLLKMI
RHTTNLTLWFEKCIVETENLEERVAVVSRIIEILQVFQELNNFNGVLEVV
SAMNSSPVYRLDHTFEQIPSRQKKILEEAHELSEDHYKKYLAKLRSINPP
CVPFFGIYLTNILKTEEGNPEVLKRHGKELINFSKRRKVAEITGEIQQYQ
NQPYCLRVESDIKRFFENLNPMGNSMEKEFTDYLFNKSLEIEPRNPKPLP
RFPKKYSYPLKSPGVRPSNPRPGT
Recombinant Human KRAS WT
SEQ ID NO: 8
GGGGTEYKLVVVGAGGVGKSALTIQLIQNHFVDEYDPTIEDSYRKQVVID
GETCLLDILDTAGQEEYSAMRDQYMRTGEGFLCVFAINNTKSFEDIHHYR
EQIKRVKDSEDVPMVLVGNKCDLPSRTVDTKQAQDLARSYGIPFIETSAK
TRQGVDDAFYTLVREIRKHKEK
SEQUENCES (Procedure B)
KRAS G12C (amino acids 1-169, N-terminal His-tag)
SEQ ID NO: 9
MASSHHHHHHSSENLYFQGMTEYKLVVVGACGVGKSALTIQLIQNHFVDE
YDPTIEDSYRKQVVIDGETCLLDILDTAGQEEYSAMRDQYMRTGEGFLCV
FAINNTKSFEDIHHYREQIKRVKDSEDVPMVLVGNKCDLPSRTVDTKQAQ
DLARSYGIPFIETSAKTRQGVDDAFYTLVREIRKHKEK
KRAS G12D (amino acids 1-169, N-terminal His-tag)
SEQ ID NO: 10
MASSHHHHHHSSENLYFQGMTEYKLVVVGADGVGKSALTIQLIQNHFVDE
YDPTIEDSYRKQVVIDGETCLLDILDTAGQEEYSAMRDQYMRTGEGFLCV
FAINNTKSFEDIHHYREQIKRVKDSEDVPMVLVGNKCDLPSRTVDTKQAQ
DLARSYGIPFIETSAKTRQGVDDAFYTLVREIRKHKEK
KRAS G12V (amino acids 1-169, N-terminal His-tag)
SEQ ID NO: 11
MGSSHHHHHHSSGLVPRGSHMASMTGGQQMGRGSENLYFQGMTEYKLVVV
GAVGVGKSALTIQLIQNHFVDEYDPTIEDSYRKQVVIDGETCLLDILDTA
GQEEYSAMRDQYMRTGEGFLCVFAINNTKSFEDIHHYREQIKRVKDSEDV
PMVLVGNKCDLPSRTVDTKQAQDLARSYGIPFIETSAKTRQGVDDAFYTL
VREIRKHKEK
KRAS WT (amino acids 1-169, N-terminal His-tag)
SEQ ID NO: 12
MASSHHHHHHSSENLYFQGMTEYKLVVVGAGGVGKSALTIQLIQNHFVDE
YDPTIEDSYRKQVVIDGETCLLDILDTAGQEEYSAMRDQYMRTGEGFLCV
FAINNTKSFEDIHHYREQIKRVKDSEDVPMVLVGNKCDLPSRTVDTKQAQ
DLARSYGIPFIETSAKTRQGVDDAFYTLVREIRKHKEK
SOS1 (amino acids 564-1049, No tag)
SEQ ID NO: 13
GEEQMRLPSADVYRFAEPDSEENIIFEENMQPKAGIPIIKAGTVIKLIER
LTYHMYADPNFVRTFLTTYRSFCKPQELLSLIIERFEIPEPEPTEADRIA
IENGDQPLSAELKRFRKEYIQPVQLRVLNVCRHWVEHHFYDFERDAYLLQ
RMEEFIGTVRGKAMKKWVESITKIIQRKKIARDNGPGHNITFQSSPPTVE
WHISRPGHIETFDLLTLHPIEIARQLTLLESDLYRAVQPSELVGSVWTKE
DKEINSPNLLKMIRHTTNLTLWFEKCIVETENLEERVAVVSRIIEILQVF
QELNNFNGVLEVVSAMNSSPVYRLDHTFEQIPSRQKKILEEAHELSEDHY
KKYLAKLRSINPPCVPFFGIYLTNILKTEEGNPEVLKRHGKELINFSKRR
KVAEITGEIQQYQNQPYCLRVESDIKRFFENLNPMGNSMEKEFTDYLFNK
SLEIEPRNPKPLPRFPKKYSYPLKSPGVRPSNPRPGT

1. A compound of Formula (I) 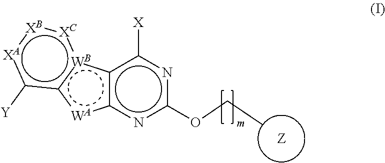
wherein:
X is: 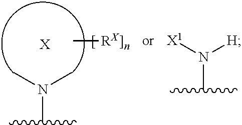
Ring X is selected from the group consisting of:
(i) a 6- to 9-membered bridged bicyclic heterocycloalkyl, wherein the bridged bicyclic heterocycloalkyl is saturated and contains one N atom and one optional O atom in addition to the illustrated N atom;
(ii) an 8- to 10-membered fused heterocycloalkyl, wherein the fused heterocycloalkyl is saturated and contains one N atom in addition to the illustrated N atom;
each R^{X} is independently selected from the group consisting of fluoro, cyano, hydroxy, oxo, C₁-C₆ alkyl, C₁-C₆ fluoroalkyl, C₁-C₆ cyanoalkyl, and C₁-C₃ hydroxyalkyl;
X¹ is —(CH₂)ₚ—C^{X}, wherein C^{X} is:
(i) a 3- to 10-membered mono- or bicyclic cycloalkyl;
(ii) a 3- to 10-membered mono- or bicyclic heterocycloalkyl, wherein the 3- to 10-membered mono- or bicyclic heterocycloalkyl is saturated and contains from 1 to 3 heteroatoms independently selected from the group consisting of N, S, and O;
wherein C^{X} is substituted by one R^{C1} which is selected from the group consisting of amino, C₁-C₃ alkylamino, and C₁-C₃ dialkylamino;
wherein C^{X} is unsubstituted or substituted by 1-3 R^{C2} substituents selected from the group consisting of fluoro, cyano, hydroxy, oxo, C₁-C₆ alkyl, C₁-C₆ fluoroalkyl, and C₁-C₃ hydroxyalkyl;
X^{A}, X^{B}, and X^{C} are independently selected from the group consisting of N, C(H), and C(R¹);
each R¹ is independently selected from the group consisting of C₁-C₃ alkyl, C₁-C₃ fluoroalkyl, halo and cyano;
W^{A} is selected from the group consisting of N(R^{W1}), C(R^{W2})₂, O, S, Se, and C(R^{W3});
R^{W1} is selected from the group consisting of C₁-C₃ alkyl, C₁-C₃ fluoroalkyl, C₃-C₆ cycloalkyl, and C₃-C₆ fluorocycloalkyl;
each R^{W2} is independently selected from the group consisting of H, halo, hydroxy, C₁-C₃ alkyl, and C₁-C₃ fluoroalkyl;
or alternatively, the two R^{W2}, together with the carbon atom to which they are attached form a C₃-C₆ cycloalkyl or C₃-C₆ fluorocycloalkyl;
R^{W3} is selected from the group consisting of H, halo, C₁-C₃ alkyl, C₁-C₃ alkoxy, C₁-C₃ fluoroalkyl, C₁-C₃ fluoroalkoxy, C₃-C₆ cycloalkyl, and C₃-C₆ fluorocycloalkyl;
W^{B} is C or N, wherein when:
W^{B} is C, then W^{A} is N(R^{W1}), C(R^{W2})₂, O, S, or Se; and
W^{B} is N, then W^{A} is C(R^{W3});
Y is C₁-C₆ alkyl, C₁-C₆ fluoroalkyl, or C^{y};
C^{y} is
(i) an aryl selected from the group consisting of phenyl or naphthyl:
(ii) a 5- to 6-membered monocyclic heteroaryl, wherein the 5- to 6-membered monocyclic heteroaryl contains 1 to 3 heteroatoms independently selected from the group consisting of N, S, and O;
(iii) a 9- to 10-membered bicyclic heteroaryl, wherein the 9- to 10-membered bicyclic heteroaryl contains 1 to 3 heteroatoms independently selected from the group consisting of N, O, and S; or
(iv) a 12- to 17-membered tri- or tetracyclic heterocycloalkyl, where at least 2 of the rings of the 12- to 17-membered tri- or tetracyclic heterocycloalkyl are aromatic, the third ring is partially unsaturated or aromatic, and the fourth ring, if present, is saturated, wherein the 12- to 17-membered tri- or tetracyclic heterocycloalkyl contains 1 to 4 heteroatoms independently selected from the group consisting of N, S, and O;
wherein C^{y} is unsubstituted or substituted by 1 to 4 R^{Y} substituents independently selected from the group consisting of halo, hydroxy, C₁-C₃ alkyl, C₂-C₄ alkynyl, C₁-C₃ fluoroalkyl, C₁-C₃ alkoxy, C₁-C₃ haloalkoxy, C₁-C₃ alkylthio, C₁-C₃ fluoroalkylthio, amino, C₁-C₃ alkylamino, C₁-C₃ dialkylamino, C₃-C₁₂ cycloalkyl, C₃-C₁₂ fluorocycloalkyl, tri(C₁-C₃ alkyl)silyl, and cyano;
Ring Z is:
(i) a 3- to 10-membered mono- or bicyclic cycloalkyl;
(ii) a 3- to 10-membered mono- or bicyclic-heterocycloalkyl, wherein the 3- to 10-membered mono- or bicyclic-heterocycloalkyl is saturated and contains 1 to 2 heteroatom groups selected from the group consisting of N, S, S(O), S(O)₂ and O; or
(iii) a 3- to 10-membered spiroheterocycloalkyl, wherein the 3- to 10-membered spiroheterocycloalkyl is saturated and contains 1 to 2 heteroatoms selected from the group consisting of N, S, and O;
wherein Ring Z is unsubstituted or substituted by 1 to 4 R^{Z} substituents independently selected from the group consisting of halo, hydroxy, oxo, C₁-C₆ alkyl, C₁-C₃ alkoxy, C₁-C₃ hydroxyalkyl, C₁-C₆ fluoroalkyl, C₁-C₃ alkylsulfonyl, C₁-C₃ alkylsulfinyl, C₁-C₃ alkylsulfonimidoyl, carboxy, carbamoyl, cyano, methoxy(C₁-C₃)alkyl, amino(C₁-C₃)alkyl, C₁-C₃ alkylamino(C₁-C₃)alkyl, C₁-C₃ dialkylamino, C₁-C₃ dialkylamino(C₁-C₃)alkyl, azetidinyl(C₁-C₃)alkyl, pyrollidinyl(C₁-C₃)alkyl, piperidinyl(C₁-C₃)alkyl, heterocyclyl(C₁-C₃)alkyl, and fluoromethylenyl;
or alternatively, two R^{Z}, together with the carbon atom to which they are attached form a C₃-C₆ cycloalkyl or C₃-C₆ fluorocycloalkyl;
wherein the heterocyclyl group of heterocyclyl(C₁-C₃)alkyl is:
(i) a 3- to 10-membered mono- or bicyclic-heterocyclyl group, wherein the 3- to 10-membered mono- or bicyclic-heterocyclyl group is saturated and contains 1 to 2 heteroatom groups selected from the group consisting of N, S, S(O), S(O)₂ and O; or
(ii) a 3- to 12-membered spiroheterocyclyl group, wherein the 3- to 10-membered spiroheterocyclyl group is saturated and contains 1 to 2 heteroatoms selected from the group consisting of N, S, and O;
wherein the heterocyclyl group of heterocyclyl(C₁-C₃)alkyl is unsubstituted or substituted by 1 to 4 R^{HC} substituents independently selected from the group consisting of halo, acyl, C₁-C₆ fluoroalkyl, C₁-C₆ alkoxyalkyl, and methyltriazolyl(C₁-C₃)alkyl;
subscript m is 0, 1, or 2;
subscript n is 0, 1, 2, or 3; and
subscript p is 0 or 1;
or a pharmaceutically acceptable salt thereof.

2. The compound of claim 1 or the pharmaceutically acceptable salt thereof, wherein X is

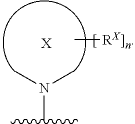

3. The compound of claim 2 or the pharmaceutically acceptable salt thereof, wherein 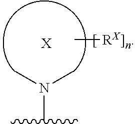
is selected from the group consisting of

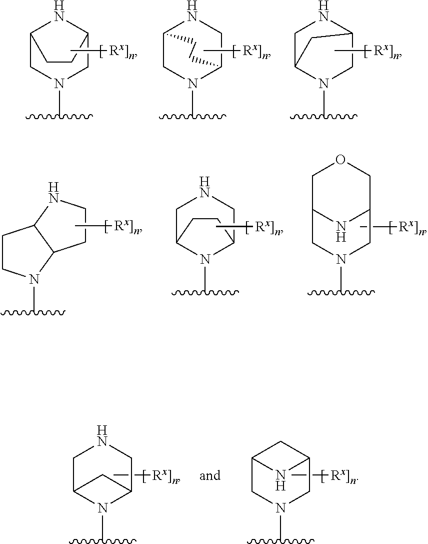

4. (canceled)

5. (canceled)

6. The compound of claim 1 or the pharmaceutically acceptable salt thereof, wherein W^{A} is N(R^{W1}).

7.-9. (canceled)

10. The compound of claim 1 or the pharmaceutically acceptable salt thereof, wherein W^{A} is S.

11. The compound of claim 1 or the pharmaceutically acceptable salt thereof, wherein:
X^{A} is N,
X^{B} and X^{C} are independently selected from the group consisting of C(H) and (CR¹); and
W^{B} is C.

12. The compound of claim 1 or the pharmaceutically acceptable salt thereof, wherein the compound of Formula (I) has the Formula (IA)

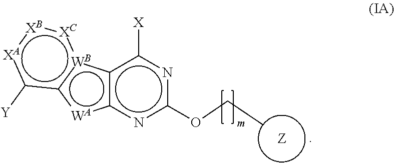

13. The compound of claim 1 or the pharmaceutically acceptable salt thereof, wherein the compound of Formula (I) has the Formula (IB)

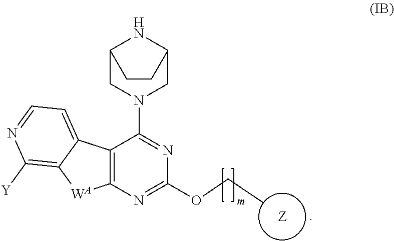

14. (canceled)

15. The compound of claim 1 or the pharmaceutically acceptable salt thereof, wherein the group 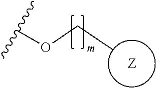
is selected from the group consisting of:

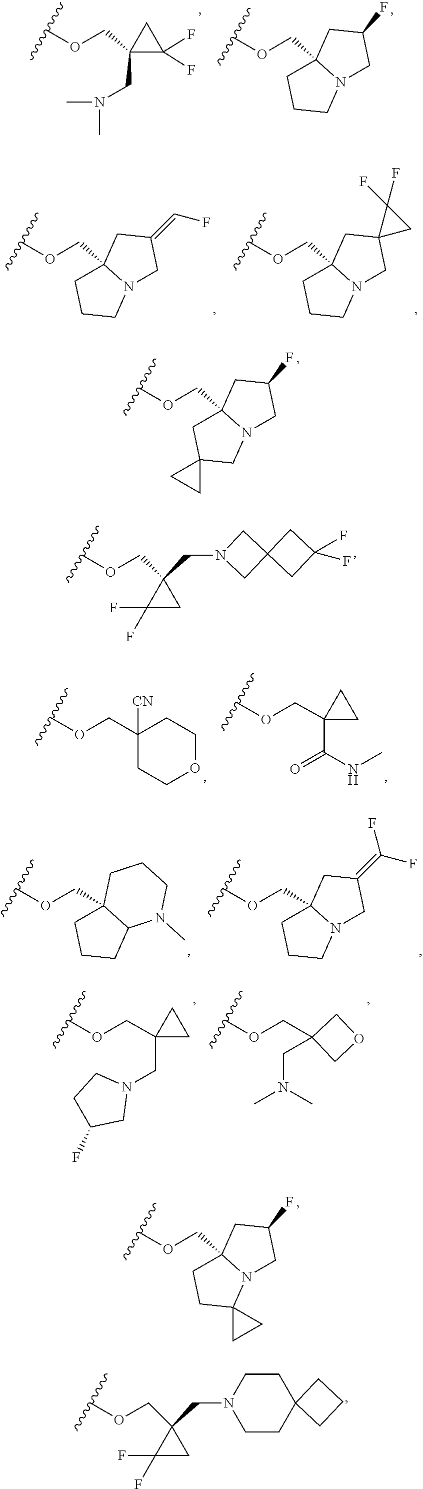

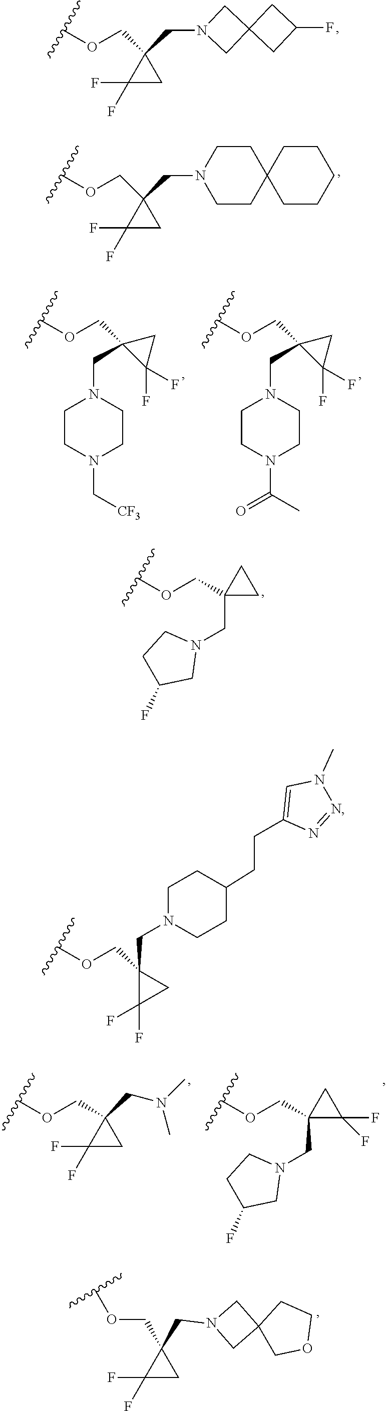

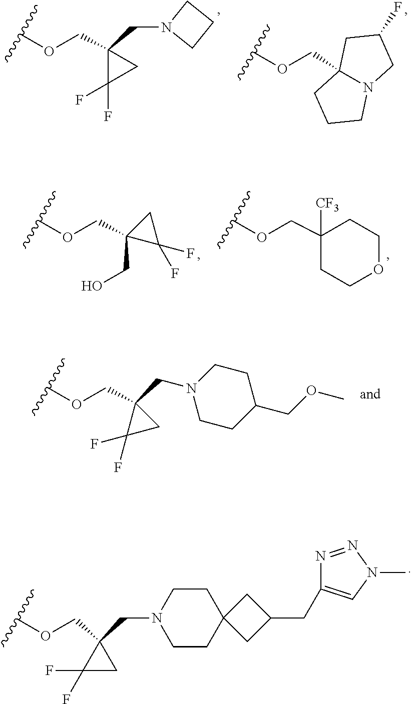 [SMILES: *OC[C@]1(CN2CCC2)CC1(F)F ; *OC[C@@]12CCCN1C[C@@H](F)C2 ; *OC[C@]1(CO)CC1(F)F ; *OCC1(C(F)(F)F)CCOCC1 ; *OC[C@]1(CN2CCC(COC)CC2)CC1(F)F ; *OC[C@]1(CN2CCC3(CC2)CC(Cc2cn(C)nn2)C3)CC1(F)F]

16. The compound of claim 1 or the pharmaceutically acceptable salt thereof, wherein the group 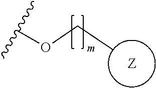
is selected from the group consisting of: 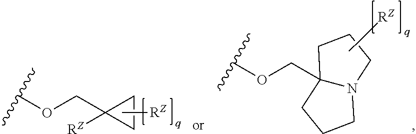
wherein subscript q is 1, 2, 3 or 4.

17. The compound of claim 16 or the pharmaceutically acceptable salt thereof, wherein the group 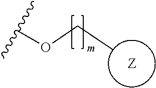
is selected from the group consisting of:

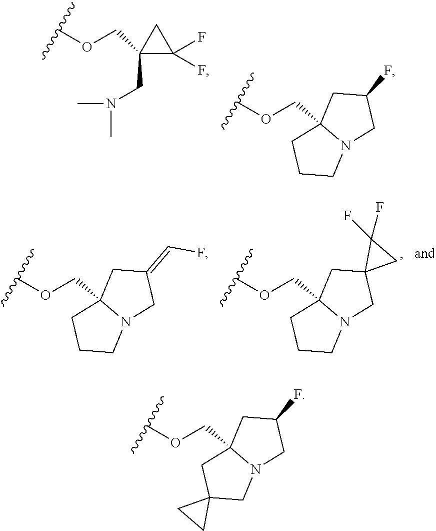

18. The compound of claim 16 or the pharmaceutically acceptable salt thereof, wherein the group 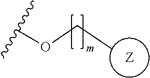
is selected from the group consisting of:

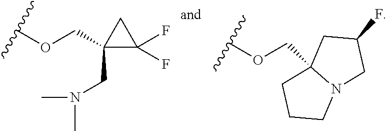 [SMILES: *OC[C@]1(CN(C)C)CC1(F)F ; *OC[C@@]12CCCN1C[C@H](F)C2]

19. (canceled)

20. (canceled)

21. The compound of claim 1 or the pharmaceutically acceptable salt thereof, wherein C^{Y} is selected from the group consisting of: 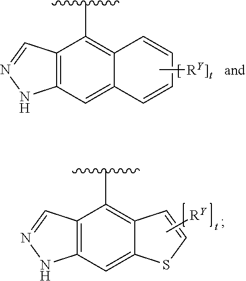
and subscript t is 0, 1, 2, 3, or 4.

22. The compound of claim 1 or the pharmaceutically acceptable salt thereof, wherein the compound is selected from the group consisting of Example Nos. 1-80 and 82-118.

23. A compound of Formula (I) 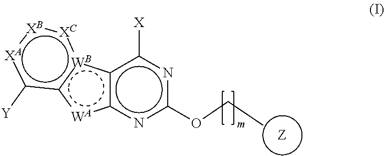
wherein:
X is: 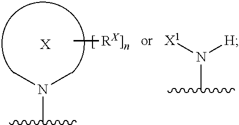
Ring X is selected from the group consisting of:
(i) a 6- to 9-membered bridged bicyclic heterocycloalkyl, wherein the bridged bicyclic heterocycloalkyl is saturated and contains one N atom and one optional O atom in addition to the illustrated N atom;
(ii) an 8- to 10-membered fused heterocycloalkyl, wherein the fused heterocycloalkyl is saturated and contains one N atom in addition to the illustrated N atom;
each R^{X} is independently selected from the group consisting of fluoro, cyano, hydroxy, oxo, C₁-C₆ alkyl, C₁-C₆ fluoroalkyl, and C₁-C₃ hydroxyalkyl;
X¹ is —(CH₂)ₚ—C^{X}, wherein C^{X} is:
(i) a 3- to 10-membered mono- or bicyclic cycloalkyl;
(ii) a 3- to 10-membered mono- or bicyclic heterocycloalkyl, wherein the 3- to 10-membered mono- or bicyclic heterocycloalkyl is saturated and contains from 1 to 3 heteroatoms independently selected from the group consisting of N, S, and O;
wherein C^{X} is substituted by one R^{C1} which is selected from the group consisting of amino, C₁-C₃ alkylamino, and C₁-C₃ dialkylamino;
wherein C^{X} is unsubstituted or substituted by 1-3 R^{C2} substituents selected from the group consisting of fluoro, cyano, hydroxy, oxo, C₁-C₆ alkyl, C₁-C₆ fluoroalkyl, and C₁-C₃ hydroxyalkyl;
X^{A}, X^{B}, and X^{C} are independently selected from the group consisting of N, C(H), and C(R¹);
each R¹ is independently selected from the group consisting of C₁-C₃ alkyl, C₁-C₃ fluoroalkyl, halo and cyano;
W^{A} is selected from the group consisting of N(R^{W1}), C(R^{W2})₂, O, S, Se, and C(R^{W3});
R^{W1} is selected from the group consisting of C₁-C₃ alkyl, C₁-C₃ fluoroalkyl, C₃-C₆ cycloalkyl, and C₃-C₆ fluorocycloalkyl;
each R^{W2} is independently selected from the group consisting of H, halo, hydroxy, C₁-C₃ alkyl, and C₁-C₃ fluoroalkyl;
or alternatively, the two R^{W2}, together with the carbon atom to which they are attached form a C₃-C₆ cycloalkyl or C₃-C₆ fluorocycloalkyl;
R^{W3} is selected from the group consisting of H, halo, C₁-C₃ alkyl, C₁-C₃ alkoxy, C₁-C₃ fluoroalkyl, C₁-C₃ fluoroalkoxy, C₃-C₆ cycloalkyl, and C₃-C₆ fluorocycloalkyl;
W^{B} is C or N, wherein when:
W^{B} is C, then W^{A} is N(R^{W1}), C(R^{W2})₂, O, or S; and
W^{B} is N, then W^{A} is C(R^{W3});
Y is C₁-C₆ alkyl, C₁-C₆ fluoroalkyl, or C^{y};
C^{y} is
(i) an aryl selected from the group consisting of phenyl or naphthyl:
(ii) a 5- to 6-membered monocyclic heteroaryl, wherein the 5- to 6-membered monocyclic heteroaryl contains 1 to 3 heteroatoms independently selected from the group consisting of N, S, and O;
(iii) a 9- to 10-membered bicyclic heteroaryl, wherein the 9- to 10-membered bicyclic heteroaryl contains 1 to 3 heteroatoms independently selected from the group consisting of N, O, and S; or
(iv) a 12- to 17-membered tri- or tetracyclic heterocycloalkyl, where at least 2 of the rings of the 12- to 17-membered tri- or tetracyclic heterocycloalkyl are aromatic, the third ring is partially unsaturated or aromatic, and the fourth ring, if present, is saturated, wherein the 12- to 17-membered tri- or tetracyclic heterocycloalkyl contains 1 to 4 heteroatoms independently selected from the group consisting of N, S, and O;
wherein C^{y} is unsubstituted or substituted by 1 to 4 R^{Y} substituents independently selected from the group consisting of halo, hydroxy, C₁-C₃ alkyl, C₂-C₄ alkynyl, C₁-C₃ fluoroalkyl, C₁-C₃ alkoxy, C₁-C₃ haloalkoxy, C₁-C₃ alkylthio, C₁-C₃ fluoroalkylthio, amino, C₁-C₃ alkylamino, C₁-C₃ dialkylamino, C₃-C₁₂ cycloalkyl, C₃-C₁₂ fluorocycloalkyl, tri(C₁-C₃ alkyl)silyl, and cyano;
Ring Z is:
(i) a 3- to 10-membered mono- or bicyclic cycloalkyl;
(ii) a 3- to 10-membered mono- or bicyclic-heterocycloalkyl, wherein the 3- to 10-membered mono- or bicyclic-heterocycloalkyl is saturated and contains 1 to 2 heteroatom groups selected from the group consisting of N, S, S(O), S(O)₂ and O; or
(iii) a 3- to 10-membered spiroheterocycloalkyl, wherein the 3- to 10-membered spiroheterocycloalkyl is saturated and contains 1 to 2 heteroatoms selected from the group consisting of N, S, and O;
wherein Ring Z is unsubstituted or substituted by 1 to 4 R^{Z} substituents independently selected from the group consisting of halo, hydroxy, oxo, C₁-C₆ alkyl, C₁-C₃ alkoxy, C₁-C₃ hydroxyalkyl, C₁-C₆ fluoroalkyl, C₁-C₃ alkylsulfonyl, C₁-C₃ alkylsulfinyl, C₁-C₃ alkylsulfonimidoyl, carboxy, carbamoyl, cyano, methoxy(C₁-C₃)alkyl, amino(C₁-C₃)alkyl, C₁-C₃ alkylamino(C₁-C₃)alkyl, C₁-C₃ dialkylamino, C₁-C₃ dialkylamino(C₁-C₃)alkyl, azetidinyl(C₁-C₃)alkyl, pyrollidinyl(C₁-C₃)alkyl, piperidinyl(C₁-C₃)alkyl, and fluoromethylenyl;
or alternatively, two R^{Z}, together with the carbon atom to which they are attached form a C₃-C₆ cycloalkyl or C₃-C₆ fluorocycloalkyl;
subscript m is 0, 1, or 2;
subscript n is 0, 1, 2, or 3; and
subscript p is 0 or 1;
or a pharmaceutically acceptable salt thereof.

24. The compound of claim 1 or the pharmaceutically acceptable salt thereof, wherein the compound is selected from the group consisting of Example Nos. 1-78.

25. A compound of formula 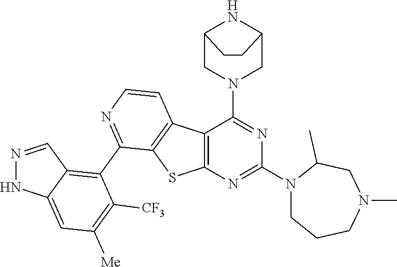 [SMILES: Cc1cc2[nH]ncc2c(-c2nccc3c2sc2nc(N4CCCN(C)CC4C)nc(N4CC5CCC(C4)N5)c23)c1C(F)(F)F]
or a pharmaceutically acceptable salt thereof.

26. A pharmaceutical composition comprising the compound of claim 1 or the pharmaceutically acceptable salt thereof, and a pharmaceutically acceptable carrier.

27. (canceled)

28. A method of inhibiting KRAS-G12D protein comprising contacting KRAS-G12D protein with the compound of claim 1, or the pharmaceutically acceptable salt thereof, to inhibit the activity of the KRAS-G12D protein.

29. A method of treating cancer comprising administering a therapeutically effective amount of the compound of 1 claim 1, or the pharmaceutically acceptable salt thereof, to a subject in need of such treatment.

30.-37. (canceled)


SEQUENCE LISTING (13 sequences)

SEQ ID NO: 1 - protein, 183 aa, synthetic construct
GLNDIFEAQKIEWHETEYKLVVVGACGVGKSALTIQLIQNHFVDEYDPTIEDSYRKQVVIDGETSLLDILDTAGQEEYSAMRDQYMRTGEGFLLVFAINNTKSFEDIHHYREQIKRVKDSEDVPMVLVGNKSDLPSRTVDTKQAQDLARSYGIPFIETSAKTRQGVDDAFYTLVREIRKHKEK

SEQ ID NO: 2 - protein, 183 aa, synthetic construct
GLNDIFEAQKIEWHETEYKLVVVGADGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 3 - protein, 183 aa, synthetic construct
GLNDIFEAQKIEWHETEYKLVVVGAVGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 4 - protein, 183 aa, synthetic construct
GLNDIFEAQKIEWHETEYKLVVVGAGDVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 5 - protein, 172 aa, synthetic construct
GGGGSHMTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQH

SEQ ID NO: 6 - protein, 176 aa, synthetic construct
GGGGMTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLN

SEQ ID NO: 7 - protein, 524 aa, synthetic construct
MGSSHHHHHHSGENLYFQGSSGLNDIFEAQKIEWHESSEEQMRLPSADVYRFAEPDSEENIIFEENMQPKAGIPIIKAGTVIKLIERLTYHMYADPNFVRTFLTTYRSFCKPQELLSLIIERFEIPEPEPTEADRIAIENGDQPLSAELKRFRKEYIQPVQLRVLNVCRHWVEHHFYDFERDAYLLQRMEEFIGTVRGKAMKKWVESITKIIQRKKIARDNGPGHNITFQSSPPTVEWHISRPGHIETFDLLTLHPIEIARQLTLLESDLYRAVQPSELVGSVWTKEDKEINSPNLLKMIRHTTNLTLWFEKCIVETENLEERVAVVSRIIEILQVFQELNNFNGVLEVVSAMNSSPVYRLDHTFEQIPSRQKKILEEAHELSEDHYKKYLAKLRSINPPCVPFFGIYLTNILKTEEGNPEVLKRHGKELINFSKRRKVAEITGEIQQYQNQPYCLRVESDIKRFFENLNPMGNSMEKEFTDYLFNKSLEIEPRNPKPLPRFPKKYSYPLKSPGVRPSNPRPGT

SEQ ID NO: 8 - protein, 172 aa, synthetic construct
GGGGTEYKLVVVGAGGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 9 - protein, 188 aa, synthetic construct
MASSHHHHHHSSENLYFQGMTEYKLVVVGACGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 10 - protein, 188 aa, synthetic construct
MASSHHHHHHSSENLYFQGMTEYKLVVVGADGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 11 - protein, 210 aa, synthetic construct
MGSSHHHHHHSSGLVPRGSHMASMTGGQQMGRGSENLYFQGMTEYKLVVVGAVGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 12 - protein, 188 aa, synthetic construct
MASSHHHHHHSSENLYFQGMTEYKLVVVGAGGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 13 - protein, 487 aa, synthetic construct
GEEQMRLPSADVYRFAEPDSEENIIFEENMQPKAGIPIIKAGTVIKLIERLTYHMYADPNFVRTFLTTYRSFCKPQELLSLIIERFEIPEPEPTEADRIAIENGDQPLSAELKRFRKEYIQPVQLRVLNVCRHWVEHHFYDFERDAYLLQRMEEFIGTVRGKAMKKWVESITKIIQRKKIARDNGPGHNITFQSSPPTVEWHISRPGHIETFDLLTLHPIEIARQLTLLESDLYRAVQPSELVGSVWTKEDKEINSPNLLKMIRHTTNLTLWFEKCIVETENLEERVAVVSRIIEILQVFQELNNFNGVLEVVSAMNSSPVYRLDHTFEQIPSRQKKILEEAHELSEDHYKKYLAKLRSINPPCVPFFGIYLTNILKTEEGNPEVLKRHGKELINFSKRRKVAEITGEIQQYQNQPYCLRVESDIKRFFENLNPMGNSMEKEFTDYLFNKSLEIEPRNPKPLPRFPKKYSYPLKSPGVRPSNPRPGT